(19) United States
(12) Patent Application Publication
(10) Pub. No.: US 2003/0087816 A1
(43) Pub. Date: May 8, 2003

(54) Novel proteins and nucleic acids encoding same

(76) Inventors: Corine Vermet, Gainesville, FL (US); Elma Fernandes, Branford, CT (US); Richard Shimkets, West Haven, CT (US); John Herrmann, Guilford, CT (US); Kumud Majumder, Stamford, CT (US); John MacDougall, Hamden, CT (US); Vishnu Mishra, Gainesville, FL (US); Peter S. Mezes, Old Lyme, CT (US); Luca Rastelli, Guilford, CT (US)

Correspondence Address: Ivor R. Elrifi, Esq., Mintz, Levin, Cohn, Ferris,, Glovsky and Popeo, P.C., One Financial Center, Boston, MA, 02111, US

(21) Appl. No.: 09/800,198

(22) Filed: Mar. 5, 2001

Related U.S. Application Data
(60) Provisional application No. 60/186,592, filed on Mar. 3, 2000.

(51) Int. Cl. A61K 38/17; C07H 21/04; C12P 21/02; C12N 5/06; C07K 14/705

(52) U.S. Cl. 514/12; 530/350; 536/23.5; 435/69.1; 435/325; 435/320.1

(57) ABSTRACT

Disclosed herein are novel human nucleic acid sequences which encode polypeptides. Also disclosed are polypeptides encoded by these nucleic acid sequences, and antibodies which immunospecifically-bind to the polypeptide, as well as derivatives, variants, mutants, or fragments of the aforementioned polypeptide, polynucleotide, or antibody. The invention further disclosed therapeutic, diagnostic and research methods for diagnosis, treatment, and prevention of disorders involving any one of these novel human nucleic acids and proteins.

### RELATED APPLICATIONS

This application claims priority from U.S. Ser. No. 60/186,592, filed Mar. 3, 2000; U.S. Ser. No. 60/186,718, filed Mar. 3, 2000; U.S. Ser. No. 60/187,293, filed Mar. 6, 2000; U.S. Ser. No. 60/187,294, filed Mar. 6, 2000; U.S. Ser. No. 60/190,400, filed Mar. 17, 2000; U.S. Ser. No. 60/196,018, filed Apr. 7, 2000; U.S. Ser. No. 60/259,548, filed Jan. 3, 2001; each of which is incorporated by reference in its entirety.

### BACKGROUND OF THE INVENTION

The invention relates generally to polynucleotides and polypeptides, as well as vectors, host cells, antibodies, and recombinant methods for producing these nucleic acids and polypeptides.

### SUMMARY OF THE INVENTION

The invention is based in part upon the discovery of novel nucleic acid sequences encoding novel polypeptides. The disclosed FCTR1, FCTR2, FCTR3, FCTR4, FCTR5, FCTR6 and FCTR7 nucleic acids and polypeptides encoded therefrom, as well as derivatives, homologs, analogs and fragments thereof, will hereinafter be collectively designated as “FCTRX” nucleic acid or polypeptide sequences.

In one aspect, the invention provides an isolated FCTRX nucleic acid molecule encoding a FCTRX polypeptide that includes a nucleic acid sequence that has identity to the nucleic acids disclosed in SEQ ID NOS: 1, 3, 5, 7, 9, 10, 11, 12, 14, 16, 18, 20, 22, and 24. In some embodiments, the FCTRX nucleic acid molecule will hybridize under stringent conditions to a nucleic acid sequence complementary to a nucleic acid molecule that includes a protein-coding sequence of a FCTRX nucleic acid sequence. The invention also includes an isolated nucleic acid that encodes a FCTRX polypeptide, or a fragment, homolog, analog or derivative thereof. For example, the nucleic acid can encode a polypeptide at least 80% identical to a polypeptide comprising the amino acid sequences of SEQ ID NOS:2, 4, 6, 8, 13, 15, 17, 19, 21, 23, and 25. The nucleic acid can be, for example, a genomic DNA fragment or a cDNA molecule that includes the nucleic acid sequence of any of SEQ ID NOS: 1, 3, 5, 7, 9, 10, 11, 12, 14, 16, 18, 20, 22, and 24.

Also included in the invention is an oligonucleotide, e.g., an oligonucleotide which includes at least 6 contiguous nucleotides of a FCTRX nucleic acid (e.g., SEQ ID NOS: 1, 3, 5, 7, 9, 10, 11, 12, 14, 16, 18, 20, 22, and 24) or a complement of said oligonucleotide.

Also included in the invention are substantially purified FCTRX polypeptides (SEQ ID NO: 2, 4, 6, 8, 13, 15, 17, 19, 21, 23, and 25). In certain embodiments, the FCTRX polypeptides include an amino acid sequence that is substantially identical to the amino acid sequence of a human FCTRX polypeptide.

The invention also features antibodies that immunoselectively-binds to FCTRX polypeptides, or fragments, homologs, analogs or derivatives thereof.

In another aspect, the invention includes pharmaceutical compositions that include therapeutically- or prophylactically-effective amounts of a therapeutic and a pharmaceutically-acceptable carrier. The therapeutic can be, e.g., a FCTRX nucleic acid, a FCTRX polypeptide, or an antibody specific for a FCTRX polypeptide. In a further aspect, the invention includes, in one or more containers, a therapeutically- or prophylactically-effective amount of this pharmaceutical composition.

In a further aspect, the invention includes a method of producing a polypeptide by culturing a cell that includes a FCTRX nucleic acid, under conditions allowing for expression of the FCTRX polypeptide encoded by the DNA. If desired, the FCTRX polypeptide can then be recovered.

In another aspect, the invention includes a method of detecting the presence of a FCTRX polypeptide in a sample. In the method, a sample is contacted with a compound that selectively binds to the polypeptide under conditions allowing for formation of a complex between the polypeptide and the compound. The complex is detected, if present, thereby identifying the FCTRX polypeptide within the sample.

The invention also includes methods to identify specific cell or tissue types based on their expression of a FCTRX.

Also included in the invention is a method of detecting the presence of a FCTRX nucleic acid molecule in a sample by contacting the sample with a FCTRX nucleic acid probe or primer, and detecting whether the nucleic acid probe or primer bound to a FCTRX nucleic acid molecule in the sample.

In a further aspect, the invention provides a method for modulating the activity of a FCTRX polypeptide by contacting a cell sample that includes the FCTRX polypeptide with a compound that binds to the FCTRX polypeptide in an amount sufficient to modulate the activity of said polypeptide. The compound can be, e.g., a small molecule, such as a nucleic acid, peptide, polypeptide, peptidomimetic, carbohydrate, lipid or other organic (carbon containing) or inorganic molecule, as further described herein.

Also within the scope of the invention is the use of a Therapeutic in the manufacture of a medicament for treating or preventing disorders or syndromes including, e.g., Colorectal cancer, adenomatous polyposis coli, myelogenous leukemia, congenital ceonatal alloimmune thrombocytopenia, multiple human solid malignancies, malignant ovarian tumours particularly at the interface between epithelia and stroma, malignant brain tumors, mammary tumors, human gliomas, astrocytomas, mixed glioma/astrocytomas, renal cells carcinoma, breast adenocarcinoma, ovarian cancer, melanomas, renal cell carcinoma, clear cell and granular cell carcinomas, autocrine/paracrine stimulation of tumor cell proliferation, autocrine/paracrine stimulation of tumor cell survival and tumor cell resistance to cytotoxic therapy, paranechmal and basement membrane invasion and motility of tumor cells thereby contributing to metastasis, tumor-mediated immunosuppression of T-cell mediated immune effector cells and pathways resulting in tumor escape from immune surveilance, neurological disorders, neurodegenerative disorders, nerve trauma, familial myelodysplastic syndrome, Charcot-Marie-Tooth neuropathy, demyelinating Gardner syndrome, familial myelodysplastic syndrome; mental health conditions, immunological disorders, allergy and infection, asthma, bronchial asthma, Avellino type eosinophilia, lung diseases, reproductive disorders, male infertility, female reproductive system disorders, male and female reproductive diseases, hemangioma, deafness, glycoprotein Ia deficiency, desmoid disease, turcot syndrome, liver cirrhosis, hepatitis C, gastric disorders, pancreatic diseases like diabetes, Schistosoma mansoni infection, Spinocerebellar ataxia, Plasmodium falciparum parasitemia, Corneal dystrophy—Groenouw type I, Corneal dystrophy—lattice type I, and Reis-Bucklers corneal dystrophy. The Therapeutic can be, e.g., a FCTRX nucleic acid, a FCTRX polypeptide, or a FCTRX-specific antibody, or biologically-active derivatives or fragments thereof.

The invention further includes a method for screening for a modulator of disorders or syndromes including, e.g., Also within the scope of the invention is the use of a Therapeutic in the manufacture of a medicament for treating or preventing disorders or syndromes including, e.g., Colorectal cancer, adenomatous polyposis coli, myelogenous leukemia, congenital ceonatal alloimmune thrombocytopenia, multiple human solid malignancies, malignant ovarian tumours particularly at the interface between epithelia and stroma, malignant brain tumors, mammary tumors, human gliomas, astrocytomas, mixed glioma/astrocytomas, renal cells carcinoma, breast adenocarcinoma, ovarian cancer, melanomas, renal cell carcinoma, clear cell and granular cell carcinomas, autocrine/paracrine stimulation of tumor cell proliferation, autocrine/paracrine stimulation of tumor cell survival and tumor cell resistance to cytotoxic therapy, paranechmal and basement membrane invasion and motility of tumor cells thereby contributing to metastasis, tumor-mediated immunosuppression of T-cell mediated immune effector cells and pathways resulting in tumor escape from immune surveilance, neurological disorders, neurodegenerative disorders, nerve trauma, familial myelodysplastic syndrome, Charcot-Marie-Tooth neuropathy, demyelinating Gardner syndrome, familial myelodysplastic syndrome; mental health conditions, immunological disorders, allergy and infection, asthma, bronchial asthma, Avellino type eosinophilia, lung diseases, reproductive disorders, male infertility, female reproductive system disorders, male and female reproductive diseases, hemangioma, deafness, glycoprotein Ia deficiency, desmoid disease, turcot syndrome, liver cirrhosis, hepatitis C, gastric disorders, pancreatic diseases like diabetes, Schistosoma mansoni infection, Spinocerebellar ataxia, Plasmodium falciparum parasitemia, Corneal dystrophy—Groenouw type I, Corneal dystrophy—lattice type I, and Reis-Bucklers corneal dystrophy. The method includes contacting a test compound with a FCTRX polypeptide and determining if the test compound binds to said FCTRX polypeptide. Binding of the test compound to the FCTRX polypeptide indicates the test compound is a modulator of activity, or of latency or predisposition to the aforementioned disorders or syndromes.

Also within the scope of the invention is a method for screening for a modulator of activity, or of latency or predisposition to an disorders or syndromes including, e.g., Also within the scope of the invention is the use of a Therapeutic in the manufacture of a medicament for treating or preventing disorders or syndromes including, e.g., Colorectal cancer, adenomatous polyposis coli, myelogenous leukemia, congenital ceonatal alloimmune thrombocytopenia, multiple human solid malignancies, malignant ovarian tumours particularly at the interface between epithelia and stroma, malignant brain tumors, mammary tumors, human gliomas, astrocytomas, mixed glioma/astrocytomas, renal cells carcinoma, breast adenocarcinoma, ovarian cancer, melanomas, renal cell carcinoma, clear cell and granular cell carcinomas, autocrine/paracrine stimulation of tumor cell proliferation, autocrine/paracrine stimulation of tumor cell survival and tumor cell resistance to cytotoxic therapy, paranechmal and basement membrane invasion and motility of tumor cells thereby contributing to metastasis, tumor-mediated immunosuppression of T-cell mediated immune effector cells and pathways resulting in tumor escape from immune surveilance, neurological disorders, neurodegenerative disorders, nerve trauma, familial myelodysplastic syndrome, Charcot-Marie-Tooth neuropathy, demyelinating Gardner syndrome, familial myelodysplastic syndrome; mental health conditions, immunological disorders, allergy and infection, asthma, bronchial asthma, Avellino type eosinophilia, lung diseases, reproductive disorders, male infertility, female reproductive system disorders, male and female reproductive diseases, hemangioma, deafness, glycoprotein Ia deficiency, desmoid disease, turcot syndrome, liver cirrhosis, hepatitis C, gastric disorders, pancreatic diseases like diabetes, Schistosoma mansoni infection, Spinocerebellar ataxia, Plasmodium falciparum parasitemia, Corneal dystrophy—Groenouw type I, Corneal dystrophy—lattice type I, and Reis-Bucklers corneal dystrophy by administering a test compound to a test animal at increased risk for the aforementioned disorders or syndromes. The test animal expresses a recombinant polypeptide encoded by a FCTRX nucleic acid. Expression or activity of FCTRX polypeptide is then measured in the test animal, as is expression or activity of the protein in a control animal which recombinantly-expresses FCTRX polypeptide and is not at increased risk for the disorder or syndrome. Next, the expression of FCTRX polypeptide in both the test animal and the control animal is compared. A change in the activity of FCTRX polypeptide in the test animal relative to the control animal indicates the test compound is a modulator of latency of the disorder or syndrome.

In yet another aspect, the invention includes a method for determining the presence of or predisposition to a disease associated with altered levels of a FCTRX polypeptide, a FCTRX nucleic acid, or both, in a subject (e.g., a human subject). The method includes measuring the amount of the FCTRX polypeptide in a test sample from the subject and comparing the amount of the polypeptide in the test sample to the amount of the FCTRX polypeptide present in a control sample. An alteration in the level of the FCTRX polypeptide in the test sample as compared to the control sample indicates the presence of or predisposition to a disease in the subject. Preferably, the predisposition includes, e.g., Also within the scope of the invention is the use of a Therapeutic in the manufacture of a medicament for treating or preventing disorders or syndromes including, e.g., Colorectal cancer, adenomatous polyposis coli, myelogenous leukemia, congenital ceonatal alloimmune thrombocytopenia, multiple human solid malignancies, malignant ovarian tumours particularly at the interface between epithelia and stroma, malignant brain tumors, mammary tumors, human gliomas, astrocytomas, mixed glioma/astrocytomas, renal cells carcinoma, breast adenocarcinoma, ovarian cancer, melanomas, renal cell carcinoma, clear cell and granular cell carcinomas, autocrine/paracrine stimulation of tumor cell proliferation, autocrine/paracrine stimulation of tumor cell survival and tumor cell resistance to cytotoxic therapy, paranechmal and basement membrane invasion and motility of tumor cells thereby contributing to metastasis, tumor-mediated immunosuppression of T-cell mediated immune effector cells and pathways resulting in tumor escape from immune surveilance, neurological disorders, neurodegenerative disorders, nerve trauma, familial myelodysplastic syndrome, Charcot-Marie-Tooth neuropathy, demyelinating Gardner syndrome, familial myelodysplastic syndrome; mental health conditions, immunological disorders, allergy and infection, asthma, bronchial asthma, Avellino type eosinophilia, lung diseases, reproductive disorders, male infertility, female reproductive system disorders, male and female reproductive diseases, hemangioma, deafness, glycoprotein Ia deficiency, desmoid disease, turcot syndrome, liver cirrhosis, hepatitis C, gastric disorders, pancreatic diseases like diabetes, Schistosoma mansoni infection, Spinocerebellar ataxia, Plasmodium falciparum parasitemia, Corneal dystrophy—Groenouw type I, Corneal dystrophy—lattice type I, and Reis-Bucklers corneal dystrophy. Also, the expression levels of the new polypeptides of the invention can be used in a method to screen for various cancers as well as to determine the stage of cancers.

In a further aspect, the invention includes a method of treating or preventing a pathological condition associated with a disorder in a mammal by administering to the subject a FCTRX polypeptide, a FCTRX nucleic acid, or a FCTRX-specific antibody to a subject (e.g., a human subject), in an amount sufficient to alleviate or prevent the pathological condition. In preferred embodiments, the disorder, includes, e.g., Also within the scope of the invention is the use of a Therapeutic in the manufacture of a medicament for treating or preventing disorders or syndromes including, e.g., Colorectal cancer, adenomatous polyposis coli, myelogenous leukemia, congenital ceonatal alloimmune thrombocytopenia, multiple human solid malignancies, malignant ovarian tumours particularly at the interface between epithelia and stroma, malignant brain tumors, mammary tumors, human gliomas, astrocytomas, mixed glioma/astrocytomas, renal cells carcinoma, breast adenocarcinoma, ovarian cancer, melanomas, renal cell carcinoma, clear cell and granular cell carcinomas, autocrine/paracrine stimulation of tumor cell proliferation, autocrine/paracrine stimulation of tumor cell survival and tumor cell resistance to cytotoxic therapy, paranechmal and basement membrane invasion and motility of tumor cells thereby contributing to metastasis, tumor-mediated immunosuppression of T-cell mediated immune effector cells and pathways resulting in tumor escape from immune surveilance, neurological disorders, neurodegenerative disorders, nerve trauma, familial myelodysplastic syndrome, Charcot-Marie-Tooth neuropathy, demyelinating Gardner syndrome, familial myelodysplastic syndrome; mental health conditions, immunological disorders, allergy and infection, asthma, bronchial asthma, Avellino type eosinophilia, lung diseases, reproductive disorders, male infertility, female reproductive system disorders, male and female reproductive diseases, hemangioma, deafness, glycoprotein Ia deficiency, desmoid disease, turcot syndrome, liver cirrhosis, hepatitis C, gastric disorders, pancreatic diseases like diabetes, Schistosoma mansoni infection, Spinocerebellar ataxia, Plasmodium falciparum parasitemia, Corneal dystrophy—Groenouw type I, Corneal dystrophy—lattice type I, and Reis-Bucklers corneal dystrophy.

In yet another aspect, the invention can be used in a method to identity the cellular receptors and downstream effectors of the invention by any one of a number of techniques commonly employed in the art. These include but are not limited to the two-hybrid system, affinity purification, co-precipitation with antibodies or other specific-interacting molecules.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION

The invention is based, in part, upon the discovery of novel nucleic acid sequences that encode novel polypeptides. The novel nucleic acids and their encoded polypeptides are referred to individually as FCTR1, FCTR2, FCTR3, FCTR4, FCTR5, FCTR6, and FCTR7. The nucleic acids, and their encoded polypeptides, are collectively designated herein as “FCTRX”.

The novel FCTRX nucleic acids of the invention include the nucleic acids whose sequences are provided in Tables 1A, 2A, 3A, 3C, 3E, 3F, 3G, 3H, 4A, 5A, 5C, 5E, 6A, 6C, and 7A inclusive (“Tables 1A-7A”), or a fragment, derivative, analog or homolog thereof. The novel FCTRX proteins of the invention include the protein fragments whose sequences are provided in Tables 1B, 2B, 3B, 3I, 4B, 5B, 5D, 6B, 6D, and 7B inclusive (“Tables 1B -7B”). The individual FCTRX nucleic acids and proteins are described below. Within the scope of this invention is a method of using these nucleic acids and peptides in the treatment or prevention of a disorder related to cell signaling or metabolic pathway modulation.

FCTR1

Novel FCTR1 is a growth factor (“FCTR”) protein related to follistatin-like gene, and mac25. FCTR1 (also referred to by proprietary accession number 58092213.0.36) is a full-length clone of 771 nucleotides, including the entire coding sequence of a 105 amino acid protein from nucleotides 438 to 753. The clone was originally obtained from thyroid gland, kidney, fetal kidney, and spleen tissues.

The nucleotide sequence of FCTR1 as presently determined is reported in Table 1A. The start and stop codons are bolded and the 5′ and 3′ untranslated regions are underlined.

TABLE 1A
FCTR1 nucleotide sequence (SEQ ID NO:1).

GGTCCTCACCCCCTTCCTCTCTCCCAGCCTCGGTGTCTGGTTACGGCTCCTCTGCTCGCATTGTGACTTTGGGCCAGGCTGGG
GGAAATGACCCGGGAGGGTCCCATGCGGCTACATAAAATTGGCAGCCTTAGAACTAGTGGGAAGGCGGGTGCGCGAAGTCGAG
GGGCGGAGAGAGGGGGCCGGAGGAGCTGCTTTCTGAATCCAAGTTCGTGGGCTCTCTCAGAAGTCCTCAGGACGGAGCAGAGG
TGGCCGGCGGGCCCGGCTGACTGCGCCTCTGCTTTCTTTCCATAACCTTTTCTTTCGGACTCGAATCACAGCTGCTGCGAAGG
GTCTAGTTCCGGACACTAGGGCCCCAGATCGTGTCACATCCATATGACACTTGGAATGTGACAGGGCAGGATGTGATCTTTGG
CTGTGAAGTGTTTGCCTACCCCATGGCCTCCATCGAGTGGAGGAAGGATGGCTTGGACATCCAGCTGCCAGGGGATGACCCCC
ACATCTCTGTGCAGTTTAGGGGTGGACCCCAGAGGTTTGAGGTGACTGCTGGCTGCAGATCCAGGCTGTGCGTCCCAGTGAT
GAGGGCACTTACCGCTGCCTTGCCCGCAATGCCCTGGGTCAAGTGGAGGCCCCTGCTAGCTTGACAGTGCTCACACCTGACCA
GCTGAACTCTACAGGCATCCCCCAGCTGCGATCACTAAACCTGGTTCCTGAGGAGGAGGCTGAGAGTGAAGAGAATGACGATT
ACTACTAGGTCCAGAGCTCTGGCC

The predicted amino acid sequence of FCTR1 protein corresponding to the foregoing nucleotide sequence is reported in Table 1B. FCTR1 was searched against other databases using SignalPep and PSort search protocols. The protein is most likely located in the cytoplasm (certainty=0.6500) and seems to have no N-terminal signal sequence. The predicted molecular weight of FCTR1 protein is 11711.8 daltons.

TABLE 1B
Encoded FCTR1 protein sequence (SEQ ID NO:2).

MASIEWRKDGLDIQLPGDDPHISVQFRGGPQRFEVTGWLQIQAVRPSDEGTYRCLARNALGQVEAPASLTVLTPDQLNSTGIP
QLRSLNLVPEEEAESEENDDYY

FCTR1 was initially identified with a TblastN analysis of a proprietary sequence file for a follistatin-like probe or homolog which was run against the Genomic Daily Files made available by GenBank. A proprietary software program (GenScan™) was used to further predict the nucleic acid sequence and the selection of exons. The resulting sequences were further modified by means of similarities using BLAST searches. The sequences were then manually corrected for apparent inconsistencies, thereby obtaining the sequences encoding the full-length protein.

In an analysis of sequence databases, it was found, for example, that the FCTR1 nucleic acid sequence has 31/71 bases (43%) identical and 46/71 bases positively alike to a Mus Musculus IGFBP-like protein (TREMBL Accession Number:BAA21725) shown in Table 1C. In all BLAST alignments herein, the “E-value” or “Expect” value is a numeric indication of the probability that the aligned sequences could have achieved their similarity to the BLAST query sequence by chance alone, within the database that was searched. For example, as shown in Table 1C, the probability that the subject (“Sbjct”) retrieved from the FCTR1 BLAST analysis, in this case the Mus Musculus IGFBP-like protein, matched the Query FCTR1 sequence purely by chance is 1.2×10⁻¹¹.

TABLE 1C
BLASTP of FCTR1 against Mus Musculus
IGFBP-like protein (SEQ ID NO:38)
PTNR:REMTREMBL-ACC:BAA21725 IGFEP-LIKE PROTEIN - MUS MUSCULUS
(MOUSE), 270 AA.
LENGTH = 270
SCORE = 161 (56.7 BITS), EXPECT = 1.2E-11, P = 1.2E-11
IDENTITIES = 31/71 (43%), POSITIVES = 46/71 (64%)

| | | | |
|---|---|---|---|
| QUERY: | 9 | DGLDIQLPGDDPHISVQFRGGPQRFEVTGWLQIQAVRPSDEGTYRCLARNALGQVEAPAS | 68 |
| | | +\|\|+ +\|\|\|\| +\|+\|\| \|\|\|\| \| \| \|+ \| +\| \|\|\| 51 \| \| \|\|+\|+ ++ + | |
| SBJCT: | 191 | EGLE-ELPGDHVNIAVQVRGGPSDHETTSWILINPLRKEDEGVYHCHAANAIGEAQSHGT | 249 |
| QUERY: | 69 | LTVLTPDQLNS 79 | |
| | | +\|\|\| ++ \| | |
| SBJCT: | 250 | VTVLDLNRYKS 260 | |

The amino acid sequence of FCTR1 also had 26/58 bases (44%) identical, and 38/58 bases (65%) positive for Mus Musculus Follistatin-like Protein shown in Table 1D.

TABLE 1D
BLASTP of FCTR1 against Mus Musculus Follistatin-like
Protein (SEQ ID NO:39)

PTNR:SPTREMBL-ACC:Q61582. FOLLISTATIN-LIKE 2 (FOLLISTATIN-LIKE PROTEIN) -
MUS MUSCULUS (MOUSE), 238 Ak.
LENGTH = 238
SCORE = 149 (52.5 BITS), EXPECT = 1.5E-10, P = 1.5E-10
IDENTITIES = 26/58 (44%), POSITIVES = 38/58 (65%)

| | | | |
|---|---|---|---|
| QUERY: | 15 | LPGDDPHISVQFRGGPQRFEVTGWLQIQAVRPSDEGTYRCLARNALGQVEAPASLTVL 72 | |
| | | \|\|\|\| ++++\| \|\|\|\|++ \|\|\|\|\|+ + + \| \| \| \| \| \|+ \|\| \| \| +\|\|+ | |
| SBJCT: | 165 | LPGDRENLAIQTRGGPEKHEVTGWVLVSPLSKEDAGEYECHASNSQGQASAAAKITVV | 222 |

The amino acid sequence of FCTR1 also had 26/58 bases (44%) identical, and 38/58 bases (65%) positive for Homo sapiens MAC25 protein shown in Table 1E.

TABLE 1E
BLASTP of FCTR1 against Homo sapiens MAC25 protein

```
PTNR:SPTREMBL-ACC:Q07822 MAC25 PROTEIN - HOMO SAPIENS (HUMAN), 277 AA.
                        LENGTH = 277
  SCORE = 149 (52.5 BITS), EXPECT = 3.2E−10, P = 3.2E−10
  IDENTITIES = 26/58 (44%), POSITIVES = 38/58 (65%)
```

| | | | | |
|---|---|---|---|---|
| QUERY: | 15 | LPGDDPHISVQFRGGPQRFEVTGWLQIQAVRPSDEGTYRCLARNALGQVEAPASLTVL | 72 | (SEQ ID NO:40) |
| | | \|\|\|\| ++++\| \|\|\|\|++ \|\|\|\|\|+ + + \| \| \| \| \| \|+ \|\| \| \| +\|\|+ | | |
| SBJCT: | 209 | LPGDRDNLAIQTRGGPEKHEVTGWVLVSPLSKEDAGEYECHASNSQGQASASAKITVV | 266 | |

The amino acid sequence of FCTR1 also had 26/58 bases (44%) identical, and 38/58 bases (65%) positive for Mus musculus MAC25 protein shown in Table 1F.

TABLE 1F
BLASTP of FCTR1 against Mus musculus MAC25 protein

```
PTNR:SPTREMBL-ACC:088812 MAC25 - MUS MUSCULUS (MOUSE), 281 AA
                        LENGTH = 281
  SCORE = 149 (52.5 BITS), EXPECT = 3.4E−10, P = 3.4E−10
  IDENTITIES = 26/58 (44%), POSITIVES = 38/58 (65%)
```

| | | | | |
|---|---|---|---|---|
| QUERY: | 15 | LPGDDPHISVQFRGGPQRFEVTGWLQIQAVRPSDEGTYRCLARNALGQVEAPASLTVL | 72 | (SEQ ID NO:41) |
| | | \|\|\|\| ++++\| \|\|\|\|++ \|\|\|\|\|+ + + \| \| \| \| \| \|+ \|\| \| \| +\|\|+ | | |
| SBJCT: | 208 | LPGDRENLAIQTRGGPEKHEVTGWVLVSPLSKEDAGEYECHASNSQGQASAAAKITVV | 265 | |

The amino acid sequence of FCTR1 also had 26/58 bases (44%) identical, and 38/58 bases (65%) positive for Homo sapiens Prostacyclin-stimulating factor shown in Table 1G.

TABLE 1G
BLASTP of FCTR1 against Homo sapiens Prostacyclin-stimulating factor

```
PTNR:SPTREMBL-ACC:Q16270 PROSTACYCLIN-STIMULATING FACTOR - HOMO SAPIENS (HUMAN), 282 AA
                        LENGTH = 282
  SCORE = 149 (52.5 BITS), EXPECT = 3.4E−10, P = 3.4E−10
  IDENTITIES = 26/58 (44%), POSITIVES = 38/58 (65%)
```

| | | | | |
|---|---|---|---|---|
| QUERY: | 15 | LPGDDPHISVQFRGGPQRFEVTGWLQIQAVRPSDEGTYRCLARNALGQVEAPASLTVL | 72 | (SEQ ID NO:42) |
| | | \|\|\|\| ++++\| \|\|\|\|++ \|\|\|\|\|+ + + \| \| \| \| \| \|+ \|\| \| \| +\|\|+ | | |
| SBJCT: | 209 | LPGDRDNLAIQTRGGPEKHEVTGWVLVSPLSKEDAGEYECHASNSQGQASASAKITVV | 266 | |

The amino acid sequence of FCTR1 also had 18/44 bases (40%) identical, and 25/44 bases (56%) positive for rat Colorectal cancer suppressor shown in Table 1H.

TABLE 1H
BLASTP of FCTR1 against rat Colorectal cancer suppressor

```
PTNR:PIR-ID:B40098 COLORECTAL CANCER SUPPRESSOR DCC - RAT (FRAGMENTS)
                        LENGTH = 144
  SCORE = 78 (27.5 BITS), EXPECT = 1.1E−05, SUM P(2) = 1.1E−05
  IDENTITIES = 18/44 (40%), POSITIVES = 25/44 (56%)
```

| | | | | |
|---|---|---|---|---|
| QUERY: | 33 | FEVTGW--LQIQAVRPSDEGTYRCLARNALGQVEAPASLTVLTP | 74 | (SEQ ID NO:43) |
| | | \|++ \| \|+\| \| \|\|\|\| \|+\|+\| \| \| ++ \| \| \| \| | | |
| SBJCT: | 101 | FQIVGGSNLRILGVVKSDEGFYQCVAENEAGNAQSSAQLIVPKP | 144 | |

```
  SCORE = 37 (13.0 BITS), EXPECT = 1.1E−05, SUM P(2) = 1.1E−05
  IDENTITIES = 8/19 (42%), POSITIVES = 12/19 (63%)
```

| | | | |
|---|---|---|---|
| QUERY: | 1 | MASIEWRKDGLDIQL-PGD | 18 |
| | | \| +\| \|+\|+ \|+ \|\|\| | |
| SBJCT: | 30 | MPTIHWQKNQQDLTPNPGD | 48 |

The amino acid sequence of FCTR1 also had 32/83 bases (38%) identical, and 45/83 bases (54%) positive to bases 55-137, and 24/68 bases (35%) identical, and 37/68 bases (54%) positive to bases 166-225 of Homo sapiens PTPsigma-(Brain) Precursor shown in Table 1I.

TABLE 1I
BLASTP of FCTR1 against Homo sapiens PTPsigma-(Brain) Precursor

```
PTNR:TREMBLNEW-ACC:AAD09360 PTPSIGMA-(BRAIN) PRECURSOR - HOMO SAPIENS (HUMAN), 1502 AA.
                        LENGTH = 1502
  SCORE = 109 (38.4 BITS), EXPECT = 0.00010, P = 0.00010
  IDENTITIES = 32/83 (38%), POSITIVES = 45/83 (54%)
```

| | | | | |
|---|---|---|---|---|
| QUERY: | 14 | QLPGDD-PHISVQFRG---GPQRFEVTGW-------LQIQAVR-PSDEGTYRCLARNALG | 61 | (SEQ ID NO:44) |
| | | \| \|\| \| ++ +\| \|\|\|\| + \| \|\| +\| \| \|\| \| \|+\|+\|++\| | | |
| SBJCT: | 55 | QATGDPKPRVTWNKKGKKVNSQRFETIEFDESAGAVLRIQPLRTPRDENVYECVAQNSVG | 114 | |

| | | | |
|---|---|---|---|
| QUERY: | 62 | QVEAPASLTVLTPDQLNSTGIPQL | 85 |
| | | ++ \| \|\|\|\| \|\|\| \| \| \| + | |
| SBJCT: | 115 | EITVHAKLTVLREDQLPS-GFPNI | 137 |

```
  SCORE = 77 (27.1 BITS), EXPECT = 0.25, P = 0.22
  IDENTITIES = 24/68 (35%), POSITIVES = 37/68 (54%)
```

| | | | |
|---|---|---|---|
| QUERY: | 4 | IEWRKDGLDIQLPGDDPHISVQFRGGPQRFEVTGWLQIQAVRPSDEGTYRCLARNALG-Q | 62 |
| | | \| \| \|\| \| + \|\| \| \| ++ +\| \|\|\|++ +\|+\| \| \|+\| \|+ \| + | |
| SBJCT: | 166 | ITWFKDFLPV-----DPSAS---NGRIKQLR-SGALQIESSEETDQGKYECVATNSAGVR | 216 |

| | | | |
|---|---|---|---|
| QUERY: | 63 | VEAPASLTV | 71 |
| | | +\|\|+\| \| | |
| SBJCT: | 217 | YSSPANLYV | 225 |

The amino acid sequence of FCTR1 also had 32/83 bases (38%) identical, and 45/83 bases (54%) positive for amino acids 55-137 and 26/69 bases (37%) identical, and 38/69 (54%) positive for amino acids 166-234 of Homo sapiens Protein-Tyrosine Phosphatase Sigma shown in Table 1J.

TABLE 1J
BLASTP of FCTR1 against Homo sapiens PTPsigma-(Brain) Precursor

```
PTNR:SPTREMBL-ACC:Q13332 PROTEIN-TYROSINE PHOSPHATASE, RECEPTOR-TYPE, S PRECURSOR
(EC 3.1.3.48) (PROTEIN-TYROSINE PHOSPHATASE SIGMA) (R-PTP-SIGMA) (PTPRS) - HOMO
SAPIENS (HUMAN), 1948 AA.
LENGTH = 1948
  SCORE = 109 (38.4 BITS), EXPECT = 0.00013, P = 0.00013
  IDENTITIES = 32/83 (38%), POSITIVES = 45/83 (54%)
```

| | | | | |
|---|---|---|---|---|
| QUERY: | 14 | QLPGDD-PHISVQFRG---GPQRFEVTGW-------LQIQAVR-PSDEGTYRCLARNALG | 61 | (SEQ ID NO:45) |
| | | \| \|\| \| ++ +\| \|\|\|\| + \|+\|\| +\| \| \|\| \| \|+\|+\|++\| | | |
| SBJCT: | 55 | QATGDPKPRVTWNKKGKKVNSQRFETIEFDESAGAVLRIQPLRTPRDENVYECVAQNSVG | 114 | |
| QUERY: | 62 | QVEAPASLTVLTPDQLNSTGIPQL 85 | | |
| | | ++ \| \|\|\|\| \|\|\| \| \| \| + | | |
| SBJCT: | 115 | EITVHAKLTVLREDQLPS-GFPNI 137 | | |

```
  SCORE = 88 (31.0 BITS), EXPECT = 0.023, P = 0.022
  IDENTITIES = 26/69 (37%), POSITIVES = 38/69 (55%)
```

| | | | |
|---|---|---|---|
| QUERY: | 4 | IEWRKDGLDIQLPGDDPHISVQFRGGPQRFEVT---GWLQIQAVRPSDEGTYRCLARNAL | 60 |
| | | \| \| \|\| \| + + \| \| \| + \|\| \| \| \|\|\|++ +\|+\| \| \|+\| \|+ | |
| SBJCT: | 166 | ITWFKDFLPVDPSASNGRIK-QLRS--ETFESTPIRGALQIESSEETDQGKYECVATNSA | 222 |
| QUERY: | 61 | G-QVEAPASLTV 71 | |
| | | \| + +\|\|+\|+\| | |
| SBJCT: | 223 | GVRYSSPANLYV 234 | |

A ClustalW analysis comparing the protein of the invention with related protein sequences is given in Table 1K, with FCTR1 shown on line 2. In the ClustalW alignment of the FCTR1 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be mutated to a much broader extent without altering protein structure or function.

[image]

[image]

IGFBP is expressed in neurostem cell and developing central nervous system. MAC-25, a follistatin like protein is a growth suppressor of osteosarcoma cells, and meningiomas. DCC is expressed in most normal tissues especially in colonic mucosa, but is deleted in colorectal cancers.

Since FCTR1 has similarity to these proteins (shown in BlastP, Tables 1C-1J, and in clustalW, Table 1K) it is likely that it has similar function. Therefore FCTR1 could function as on or more of the following: a tumor suppressor geneor regulator of neurological system development.

Based on the protein similarity and tissue expression, FCTR1 may be useful in the following diseases and uses:

(i) Tissue regeneration in vitro and in vivo

(ii) Neurological disorders, neurodegenerative disorders, nerve trauma

(iii) Reproductive health

(iv) Immunological disorders, allergy and infection

(v) In cancer as a diagnostic and prognostic marker, as well as a protein therapeutic

FCTR2

FCTR2 (alternatively referred to herein as AC012614_{—}1.0.123), is a growth factor bearing sequence similarity to human KIAA1061 protein and to genes involved in neuronal development and reproductive physiology (e.g., cell adhesion molecules, follistatin, roundabout and frazzled). FCTR2 is a full-length clone of 5502 nucleotides, including the entire coding sequence of a 815 amino acid protein. This sequence is expressed in glioma, osteoblast, other cancer cells, lung carcinoma, small intestine (This sequence maps to Unigene Hs. 123420 which is expressed in brain, breast, kidney, pancreas, pooled tissue).

A FCTR2 ORF begins with an ATG initiation codon at nucleotides 420422 and ends with a TGA codon at nucleotides 2865-2867. Putative untranslated regions upstream from the initiation codon and downstream from the termination codon are underlined in Table 2A, and the start and stop codons are in bold letters.

TABLE 2A
FCTR2 Nucleotide Sequence.

| | |
|---|---|
| CAATTTCACACAGGAAACAGCTATGCCATGATTACGCAAGTTGGTACCGAGCTCGGATCCACTAGTAACGGCCGCCAGTG | (SEQ ID NO:3) |
| TGCTGGAATTCGGCTTACTCACTATAGGGCTCGAGCGGCTGCCCGGGCAGGTCATTAATTCCATTTCTTTTTAGAGTATC | |
| ACAGCTTTCTCCTTCACTGACCACCCTTTGCTTCCTGTCAGAAAGCCCTGGACAGAACTCTCTGTGGGATTCTGCCCATG | |
| TTTCTGAGATATCGCCTCAATTGTCCTGGCTGGGCTGTCGGGTCTGCCCGTTTTACAGATGGGCAAACTGGAGTGGGAAG | |
| TATCCGGGTGGCTTCCTCAGGCCTGCAGCTGGTGGAGCAGCTACTGAAACAATCAGGAGCCCAGAAGCTTTGAAGTCACA | |
| AGAAGAGAAGACTCCCAGAATGCAGTGTGATGTTGGTGATGGACGCCTGTTTCGCCTTTCACTTAAACGTGCCCTTTCCA | |
| GCTGCCCTGACCTCTTTGGGCTTTCCAGCCGCAACGAGCTGCTGGCCTCCTGCGGGAAGAAGTTCTGCAGCCGAGGGAGC | |
| CGGTGCGTGCTCAGCAGGAAGACAGGGGAGCCCGAATGCCAGTGCCTGGAGGCATGCAGGCCCAGCTACGTGCCTGTGTG | |
| CGGCTCTGATGGGAGGTTTTATGAAAACCACTGTAAGCTCCACCGTGCTGCTTGCCTCCTGGGAAAGAGGATCACCGTCA | |
| TCCACAGCAAGGACTGTTTCCTCAAAGGTGACACGTGCACCATGGCCGGCTACGCCCGCTTGAAGAATGTCCTTCTGGCA | |
| CTCCAGACCCGTCTGCAGCCACTCCAAGAAGGAGACAGCAGACAAGACCCTGCCTCCCAGAAGCGCCTCCTGGTGCAATC | |
| TCTGTTCAGGGACTTAGATGCAGATGGCAATGGCCACCTCAGCAGCTCCGAACTGGCTCAGCATGTGCTGAAGAAGCAGG | |
| ACCTGGATGAAGACTTACTTGGTTGCTCACCAGGTGACCTCCTCCGATTTGACGATTACAACAGTGACAGCTCCCTGACC | |
| CTCCGCGAGTTCTACATGGCCTTCCAAGTGGTTCAGCTCAGCCTCGCCCCCGAGGACAGGGTCAGTGTGACCACAGTGAC | |
| CGTGGGGCTGAGCACAGTGCTGACCTGCGCCGTCCATGGAGACCTGAGGCCACCAATCATCTGGAAGCGCAACGGGCTCA | |
| CCCTGAACTTCCTGGACTTGGAAGACATCAATGACTTTGGAGAGGATGATTCCCTGTACATCACCAAGGTGACCACCATC | |
| CACATGGGCAATTACACCTGCCATGCTTCCGGCCACGAGCAGCTGTTCCAGACCCACGTCCTGCAGGTGAATGTGCCGCC | |
| AGTCATCCGTGTCTATCCAGAGAGCCAGGCACAGGAGCCTGGAGTGGCAGCCAGCCTAAGATGCCATGCTGAGGGCATTC | |
| CCATGCCCAGAATCACTTGGCTGAAAAACGGCGTGGATGTCTCAACTCAGATGTCCAAACAGCTCTCCCTTTTAGCCAAT | |
| GGGAGCGAACTCCACATCAGCAGTGTTCGGTATGAAGACACAGGGGCATACACCTGCATTGCCAAAAATGAAGTGGGTGT | |
| GGATGAAGATATCTCCTCGCTCTTCATTGAAGACTCAGCTAGAAAGACCCTTGCAAACATCCTGTGGCGAGAGGAAGGCC | |
| TCAGCGTGGGAAACATGTTCTATGTCTTCTCCGACGACGGTATCATCGTCATCCATCCTGTGGACTGTGAGATCCAGAGG | |
| CACCTCAAACCCACGGAAAAGATTTTCATCAGCTATGAAGAAATCTGTCCTCAAAGAGAAAAAAATGCAACCCAGCCCTG | |
| CCAGTGGGTATCTGCAGTCAATGTCCGGAACCGGTACATCTATGTGGCCCAGCCAGCACTGAGCAGAGTCCTTGTGGTCG | |
| ACATCCAAGCCCAGAAAGTCCTACAGTCCATAGGTGTGGACCCTCTGCCGGCTAAGCTGTCCTATGACAAGTCACATGAC | |
| CAAGTGTGGGTCCTGAGCTGGGGGGACGTGCACAAGTCCCGACCAAGTCTCCAGGTGATCACAGAAGCCAGCACCGGCAA | |
| GAGCCAGCACCTCATCCGCACACCCTTTGCAGGAGTGGATGATTTCTTCATTCCCCCAACAAACCTCATCATCAACCACA | |
| TCAGGTTTGGCTTCATCTTCAACAAGTCTGATCCTGCAGTCCACAAGGTGGACCTGGAAACAATGATGCCCCTCAAGACC | |
| ATCGGCCTGCACCACCATGGCTGCGTGCCCCAGGCCATGGCACACACCCACCTGGGCGGCTACTTCTTCATCCAGTGCCG | |
| ACAGGACAGCCCCGCCTCTGCTGCCCGACAGCTGCTCGTTGACAGTGTCACAGACTCTGTGCTTGGCCCCAATGGTGATG | |
| TAACAGGCACCCCACACACATCCCCCGACGGGCGCTTCATAGTCAGTGCTGCAGCTGACAGCCCCTGGCTGCACGTGCAG | |
| GAGATCACAGTGCGGGGCGAGATCCAGACCCTGTATGACCTGCAAATAAACTCGGGCATCTCAGACTTGGCCTTCCAGCG | |
| CTCCTTCACTGAAAGCAATCAATACAACATCTACGCGGCTCTGCACACGGAGCCGGACCTGCTGTTCCTGGAGCTGTCCA | |
| CGGGGAAGGTGGGCATGCTGAAGAACTTAAAGGAGCCACCCGCAGGGCCAGCTCAGCCCTGGGGGGGTACCCACAGAATC | |
| ATGAGGGACAGTGGGCTGTTTGGACAGTACCTCCTCACACCAGCCCGAGAGTCACTGTTCCTCATCAATGGGAGACAAAA | |
| CACGCTGCGGTGTGAGGTGTCAGGTATAAAGGGGGGGACCACAGTGGTGTGGGTGGGTGAGGTATGAAGGGCCCAGAGCA | |
| GAGCCCTGGGCCAAGGAACACCCCCTAGTCCTGACACTGCAGCCTCAAGCAGGTACGCTGTACATTTTTACAGACAAAAG | |
| CAAAAACCTGTACTCGCTTTGTGGTTCAACACTGGTCTCCTTGCAAGTTTCCTAGTATAAGGTATGCGCTGCTACCAAGA | |
| TTGGGGTTTTTTCGTTAGGAAGTATGATTTATGCCTTGAGCTACGATGAGAACATATGCTGCTGTGTAAAGGGATCATTT | |
| CTGTGCCAAGCTGCACACCGAGTGACCTGGGGACATCATGGAACCAAGGGATCCTGCTCTCCAAGCAGACACCTCTGTCA | |
| GTTGCCTTCACATAGTCATTGTCCCTTACTGCCAGACCCAGCCAGACTTTGCCCTGACGGAGTGGCCCGGAAGCAGAGGC | |
| CGACCAGGAGCAGGGGCCTCCCTCCCGAACTGAAAGCCCATCCGTCCTCGCGTGGGACCGCATCTTCTCCCTCGCAGCTG | |
| CTTCTTGCTTTTCTTTCCATTTGACTTGCTGTAAGCCTGAGGGAGAGCCAACAAGACTTACTGCATCTTGGGGGATGGGG | |
| AAATCACTCACTTTATTTTGGAAATTTTTGATTAAAAAAAAATTTTATAATCTCAAATGCTAGTAAGCAGAAAGATGCTC | |
| TCCGAGGTCCAACTATATCCTTCCCTGCCTTAGGCCGAGTCTCGGGGGTGGTCACAACCCCACATCCCACAGCCAGAAAG | |
| AACAATGGTCATCTGAGAATACTGGCCCTGTCGACTATTGCCACCCTGCTTCTCCAAGAGCAGACCAGGCCACCTCATCC | |
| GTAAGGACTCGGTTCTGTGTTGGGACCCCAAAAAACCAGAACAAGTTCTGTGTGCCTCCTTTCAGCACAGAAGGGAGACA | |
| TCTCATTAGTCAGGTCTGGTACCCCAGATTCAGGGCAGACTGGGCTTGCCTGGCAAGGTATGGGTGGCCTCCAGGCTCAA | |
| TGCAGAAACCCCAAGGACACGAGTGGGGCCAGGTGAGTTCCTGAAGCTATACCTTTTCAAAACAGATTTTGTTTTCCTAC | |
| CTGTGGCCCATCCACTCCTCTCTGGTACCCCATCCCCGCATCAGCACTGCAGAGAGAACACATTTCGGCGAGGGTTTTCT | |
| TACCCACATTCCCCAATCAATACACACACACTGCAGAACCCAGAACAGAAGGCCACAGGCTGGCACTACTGCATTCTCCT | |
| TATGTGTCTCAGGCTGTGGTGACTCTCACATGGGCATCGAAGAAGTACAACCCACATAGCCCTCTGGAGACCGCCTAGAT | |
| CAGAGACTCAGCAAAAACAGGCTCGCCTTCCCTCTCCCACATATGAGTGGAACTTACATGTGTCCTGGTTTGAATGATCA | |
| TTTTGCAAGCCACACGGGTTGGGAGAGGTGGTCTCACCACAGACGTCTTTGCTAATTTGGCCACCTTCACCTACTGACAT | |
| GACCAGGATTTTCCTTTGCCATTAAGGAATGAACTCTTTCAAGGAGAGGAAACCCTAGACTCTGTGTCACTCTCAACACA | |
| CACAGCTCCTTTCACTCCTGCCTGACTGCCAAGCCACCTGCATCCCCCGCCCCAGATCTCATGAGATCAATCACTTGTAT | |
| GTCTCACGCAACTTGGTCCACCAAACGCCTGTCCCCTGTAACTCCTAGGGGTGCGCCTAGACAGGTACGTCTGTTTTTTA | |
| TTTTAAAAGATATGCTATGTAGATATAAGTTGAGGAAGCTCACCTCAAAAGCCTAGAATGCAGTTTCACAGTAGCTGGGA | |
| TGCATGGATGACCCATCTCACCCCTTTTTTTTTCCTGCCTCAATATCTTGATATGTTATGTTTACTCCCAATCTCCCATT | |
| TTTACCACTAAAATTCTCCAACTTTCATAAACTTTTTTTTGGAAAAATTTCCATTGTATCAGCCCCTGACAGAAAAAGGA | |
| TCTCTGAGCCTAAAGGAGGAAAAGTCCCACCAACTACCAGACCAGAACACGAGCCCCTCTGGGCAGCAGGATTCCTAAGT | |
| CAAAGACCAGTTTGACCCAAACTGGCCTTTTAAAATAATCAGGAGTGACAGAGTCAACTTCTGCAGCACCTGCTTCTCCC | |
| CCACTGTCCCTTCCATCTTGGAATGTGTCTAAAAAAGCATAGCTGCCCTTTGCTGTCCTCAGAGTGCATTTCCTGGAGAC | |
| GGCAGGCTTAGGTCTCACTGACAGCATGCCAGACACAACTGAATCGAAGCAGGCCTGAAGCCTAGGTCAGGGTTTCAGGA | |
| GTCCAGCCCCAGGAGGCAAAGTCACCAATGCAGGGAGGTAAATGCCTTTTGGCAGGAAAACCAATAGAGTTGGTTGGGTG | |
| GGGAGTCAGGGGTGGGAGGAGAAGGAGGAAGAGGAGGAAGGCCAGACTGGCCTGCCCTTTCTCCCATACTTCACCCCAGC | |
| AGAGGTTCATGGGACACAGTTGGAAAGCCACTGGGAGGAAATGCCTCACTACAGGGGGGCCTCCTGTAGCAAGCCCAGCC | |
| GGTAATCCTCCTAATGAACCCACAAGGTCAATTCACAACTGATATCTTAGCTATTAAAGAAGTACTGACTTTACCAAAAG | |
| AATCATCAAGAAAGCTATTTATATAAACCCCCTCAGTCATTTTGAAATAAAATTAATTTTAC | |

The predicted amino acid sequence of FCTR2 protein corresponding to the foregoing nucleotide sequence is reported in Table 2B. FCTR2 was searched against other databases using SignalPep and PSort search protocols. The protein is most likely located in the mitochondrial matrix space (certainty=0.4718) and seems to have no N-terminal signal sequence. The predicted molecular weight is 90346.9 Daltons.

TABLE 2B
FCTR2 Protein Sequence.

| | |
|---|---|
| MQCDVGDGRLFRLSLKRALSSCPDLFGLSSRNELLASCGKKFCSRGSRCVLSRKTGEPECQCLEACRPSYVPVCGSDGRFYEN | (SEQ ID NO:4) |
| HCKLHRAACLLGKRITVIHSKDCFLKGDTCTMAGYARLKNVLLALQTRLQPLQEGDSRQDPASQKRLLVESLFRDLDADGNGH | |
| LSSSELAQHVLKKQDLDEDLLGCSPGDLLRFDDYNSDSSLTLREFYMAFQVVQLSLAPEDRVSVTTVTVGLSTVLTCAVHGDL | |
| RPPIIWKRNGLTLNFLDLEDINDFGEDDSLYITKVTTIHMGNYTCHASGHEQLFQTHVLQVNVPPVIRVYPESQAQEPGVAAS | |
| LRCHAEGIPMPRITWLKNGVDVSTQMSKQLSLLANGSELHISSVRYEDTGAYTCIAKNEVGVDEDISSLFIEDSARKTLANIL | |
| WREEGLSVGNMFYVFSDDGIIVIHPVDCEIQRHLKPTEKIFMSYEEICPQREKNATQPCQWVSAVNVRNRYIYVAQPALSRVL | |
| VVDIQAQKVLQSIGVDPLPAKLSYDKSHDQVWVLSWGDVHKSRPSLQVITEASTGQSQHLIRTPFAGVDDFFIPPTNLIINHI | |
| RFGFIFNKSDPAVHKVDLETMMPLKTIGLHHHGCVPQAMAHTHLGGYFFIQCRQDSPASAARQLLVDSVTDSVLGPNGDVTGT | |
| PHTSPDGRFIVSAAADSPWLHVQEITVRGEIQTLYDLQINSGISDLAFQRSFTESNQYNIYAALHTEPDLLFLELSTGKVGML | |
| KNLKEPPAGPAQPWGGTHRIMRDSGLFGQYLLTPARESLFLINGRQNTLRCEVSGIKGGTTVVWVGEV | |

In a BLASTN search it was also found that nucleotides 784-5502 of FCTR2 nucleic acid had 4672 of 4719 bases (99%) identical to Homo sapiens mRNA for KIAA1061 protein, partial cds (GenBank Acc:AB028984) (Table 2C).

TABLE 2C
BLASTN of FCTR2 against Homo sapiens mRNA for KIAA1061 protein

```
>GI|5689458|DBJ|AB028984.1/AB028984 HOMO SAPIENS MRNA FOR KIAA1061 PROTEIN, PARTIAL CDS
                    LENGTH = 4719
  SCORE = 9075 BITS (4578), EXPECT = 0.0
  IDENTITIES = 4672/4719 (99%)
  STRAND = PLUS/PLUS
```

| | | | | |
|---|---|---|---|---|
| QUERY: | 784 | AGAATGTCCTTCTGGCACTCCAGACCCGTCTGCAGCCACTCCAAGAAGGAGACAGCAGAC | 843 | (SEQ ID NO:46) |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 1 | AGAATGTCCTTCTGGCACTCCAGACCCGTCTGCAGCCACTCCAAGAAGGAGACAGCAGAC | 60 | |
| QUERY: | 844 | AAGACCCTGCCTCCCAGAAGCGCCTCCTGGTGGAATCTCTGTTCAGGGACTTAGATGCAG | 903 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 61 | AAGACCCTGCCTCCCAGAAGCGCCTCCTGGTGGAATCTCTGTTCAGGGACTTAGATGCAG | 120 | |
| QUERY: | 904 | ATGGCAATGGCCACCTCAGCAGCTCCGAACTGGCTCAGCATGTGCTGAAGAAGCAGGACC | 963 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 121 | ATGGCAATGGCCACCTCAGCAGCTCCGAACTGGCTCAGCATGTGCTGAAGAAGCAGGACC | 180 | |
| QUERY: | 964 | TGGATGAAGACTTACTTGGTTGCTCACCAGGTGACCTCCTCCGATTTGACGATTACACAA | 1023 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 181 | TGGATGAAGACTTACTTGGTTGCTCACCAGGTGACCTCCTCCGATTTGACGATTACAACA | 240 | |
| QUERY: | 1024 | GTGACAGCTCCCTGACCCTCCGCGAGTTCTACATGGCCTTCCAAGTGGTTCAGCTCAGCC | 1083 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 241 | GTGACAGCTCCCTGACCCTCCGCGAGTTCTACATGGCCTTCCAAGTGGTTCAGCTCAGCC | 300 | |
| QUERY: | 1084 | TCGCCCCCGAGGACAGGGTCAGTGTGACCACAGTGACCGTGGGGCTGAGCACAGTGCTGA | 1143 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 301 | TCGCCCCCGAGGACAGGGTCAGTGTGACCACAGTGACCGTGGGGCTGAGCACAGTGCTGA | 360 | |
| QUERY: | 1144 | CCTGCGCCGTCCATGGAGACCTGAGGCCACCAATCATCTGGAAGCGCAACGGGCTCACCC | 1203 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 361 | CCTGCGCCGTCCATGGAGACCTGAGGCCACCAATCATCTGGAAGCGCAACGGGCTCACCC | 420 | |
| QUERY: | 1204 | TGAACTTCCTGGACTTGGAAGACATCAATGACTTTGGAGAGGATGATTCCCTGTACATCA | 1263 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 421 | TGAACTTCCTGGACTTGGAAGACATCAATGACTTTGGAGAGGATGATTCCCTGTACATCA | 480 | |
| QUERY: | 1264 | CCAAGGTGACCACCATCCACATGGGCAATTACACCTGCCATGCTTCCGGCCACGAGCAGC | 1323 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 481 | CCAAGGTGACCACCATCCACATGGGCAATTACACCTGCCATGCTTCCGGCCACGAGCAGC | 540 | |
| QUERY: | 1324 | TGTTCCAGACCCACGTCCTGCAGGTGAATGTGCCGCCAGTCATCCGTGTCTATCCAGAGA | 1383 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 541 | TGTTCCAGACCCACGTCCTGCAGGTGAATGTGCCGCCAGTCATCCGTGTCTATCCAGAGA | 600 | |
| QUERY: | 1384 | GCCAGGCACAGGAGCCTGGAGTGGCAGCCAGCCTAAGATGCCATGCTGAGGGCATTCCCA | 1443 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 601 | GCCAGGCACAGGAGCCTGGAGTGGCAGCCAGCCTAAGATGCCATGCTGAGGGCATTCCCA | 660 | |
| QUERY: | 1444 | TGCCCAGAATCACTTGGCTGAAAAACGGCGTGGATGTCTCAACTCAGATGTCCAAACAGC | 1503 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 661 | TGCCCAGAATCACTTGGCTGAAAAACGGCGTGGATGTCTCAACTCAGATGTCCAAACAGC | 720 | |
| QUERY: | 1504 | TCTCCCTTTTAGCCAATGGGAGCGAACTCCACATCAGCAGTGTTCGGTATGAAGACACAG | 1563 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 721 | TCTCCCTTTTAGCCAATGGGAGCGAACTCCACATCAGCAGTGTTCGGTATGAAGACACAG | 780 | |
| QUERY: | 1564 | GGGCATACACCTGCATTGCCAAAAATGAAGTGGGTGTGGATGAAGATATCTCCTCGCTCT | 1623 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 781 | GGGCATACACCTGCATTGCCAAAAATGAAGTGGGTGTGGATGAAGATATCTCCTCGCTCT | 840 | |
| QUERY: | 1624 | TCATTGAAGACTCAGCTAGAAAGACCCTTGCAAACATCCTGTGGCGAGAGGAAGGCCTCA | 1683 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 841 | TCATTGAAGACTCAGCTAGAAAGACCCTTGCAAACATCCTGTGGCGAGAGGAAGGCCTCA | 900 | |
| QUERY: | 1684 | GCGTGGGAAACATGTTCTATGTCTTCTCCGACGACGGTATCATCGTCATCCATCCTGTGG | 1743 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 901 | GCGTGGGAAACATGTTCTATGTCTTCTCCGACGACGGTATCATCGTCATCCATCCTGTGG | 960 | |
| QUERY: | 1744 | ACTGTGAGATCCAGAGGCACCTCAAACCCACGGAAAAGATTTTCATGAGCTATGAAGAAA | 1803 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 961 | ACTGTGAGATCCAGAGGCACCTCAAACCCACGGAAAAGATTTTCATGAGCTATGAAGAAA | 1020 | |
| QUERY: | 1804 | TCTGTCCTCAAAGAGNNNNNNNTGCAACCCAGCCCTGCCAGTGGGTATCTGCAGTCAATG | 1863 | |
| | | \|\|\|\|\|\|\|\|\|\|\|\|\|\| \|\|\|\|\|\|\|\|\|\|\|\|\|\|\|\|\|\|\|\|\|\|\|\|\|\|\|\|\|\|\|\|\|\|\|\|\|\| | | |
| SBJCT: | 1021 | TCTGTCCTCAAAGAGAAAAAAATGCAACCCAGCCCTGCCAGTGGGTATCTGCAGTCAATG | 1080 | |
| QUERY: | 1864 | TCCGGAACCGGTACATCTATGTGGCCCAGCCAGCACTGAGCAGAGTCCTTGTGGTCGACA | 1923 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 1081 | TCCGGAACCGGTACATCTATGTGGCCCAGCCAGCACTGAGCAGAGTCCTTGTGGTCGACA | 1140 | |
| QUERY: | 1924 | TCCAAGCCCAGAAAGTCCTACAGTCCATAGGTGTGGACCCTCTGCCGGCTAAGCTGTCCT | 1983 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 1141 | TCCAAGCCCAGAAAGTCCTACAGTCCATAGGTGTGGACCCTCTGCCGGCTAAGCTGTCCT | 1200 | |
| QUERY: | 1984 | ATGACAAGTCACATGACCAAGTGTGGGTCCTGAGCTGGGGGGACGTGCACAAGTCCCGAC | 2043 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 1201 | ATGACAAGTCACATGACCAAGTGTGGGTCCTGAGCTGGGGGGACGTGCACAAGTCCCGAC | 1260 | |
| QUERY: | 2044 | CAAGTCTCCAGGTGATCACAGAAGCCAGCACCGGCCAGAGCCAGCACCTCATCCGCACAC | 2103 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 1261 | CAAGTCTCCAGGTGATCACAGAAGCCAGCACCGGCCAGAGCCAGCACCTCATCCGCACAC | 1320 | |
| QUERY: | 2104 | CCTTTGCAGGAGTGGATGATTTCTTCATTCCCCCAACAAACCTCATCATCAACCACATCA | 2163 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 1321 | CCTTTGCAGGAGTGGATGATTTCTTCATTCCCCCAACAAACCTCATCATCAACCACATCA | 1380 | |
| QUERY: | 2164 | GGTTTGGCTTCATCTTCAACAAGTCTGATCCTGCAGTCCACAAGGTGGACCTGGAAACAA | 2223 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 1381 | GGTTTGGCTTCATCTTCAACAAGTCTGATCCTGCAGTCCACAAGGTGGACCTGGAAACAA | 1440 | |
| QUERY: | 2224 | TGATGCCCCTCAAGACCATCGGCCTGCACCACCATGGCTGCGTGCCCCAGGCCATGGCAC | 2283 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 1441 | TGATGCCCCTCAAGACCATCGGCCTGCACCACCATGGCTGCGTGCCCCAGGCCATGGCAC | 1500 | |
| QUERY: | 2284 | ACACCCACCTGGGCGGCTACTTCTTCATCCAGTGCCGACAGGACAGCCCCGCCTCTGCTG | 2343 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 1501 | ACACCCACCTGGGCGGCTACTTCTTCATCCAGTGCCGACAGGACAGCCCCGCCTCTGCTG | 1560 | |
| QUERY: | 2344 | CCCGACAGCTGCTCGTTGACAGTGTCACAGACTCTGTGCTTGGCCCCAATGGTGATGTAA | 2403 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 1561 | CCCGACAGCTGCTCGTTGACAGTGTCACAGACTCTGTGCTTGGCCCCAATGGTGATGTAA | 1620 | |
| QUERY: | 2404 | CAGGCACCCCACACACATCCCCCGACGGGCGCTTCATAGTCAGTGCTGCAGCTGACAGCC | 2463 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 1621 | CAGGCACCCCACACACATCCCCCGACGGGCGCTTCATAGTCAGTGCTGCAGCTGACAGCC | 1680 | |
| QUERY: | 2464 | CCTGGCTGCACGTGCAGGAGATCACAGTGCGGGGCGAGATCCAGACCCTGTATGACCTGC | 2523 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 1681 | CCTGGCTGCACGTGCAGGAGATCACAGTGCGGGGCGAGATCCAGACCCTGTATGACCTGC | 1740 | |
| QUERY: | 2524 | AAATAAACTCGGGCATCTCAGACTTGGCCTTCCAGCGCTCCTTCACTGAAAGCAATCAAT | 2583 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 1741 | AAATAAACTCGGGCATCTCAGACTTGGCCTTCCAGCGCTCCTTCACTGAAAGCAATCAAT | 1800 | |
| QUERY: | 2584 | ACAACATCTACGCGGCTCTGCACACGGAGCCGGACCTGCTGTTCCTGGAGCTGTCCACGG | 2643 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 1801 | ACAACATCTACGCGGCTCTGCACACGGAGCCGGACCTGCTGTTCCTGGAGCTGTCCACGG | 1860 | |
| QUERY: | 2644 | GGAAGGTGGGCATGCTGAAGAACTTAAAGGAGCCACCCGCAGGGCCAGCTCAGCCCTNNN | 2703 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 1861 | GGAAGGTGGGCATGCTGAAGAACTTAAAGGAGCCACCCGCAGGGCCAGCTCAGCCCTGGG | 1920 | |
| QUERY: | 2704 | NNNNTACCCACAGAATCATGAGGGACAGTGGGCTGTTTGGACAGTACCTCCTCACACCAG | 2763 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 1921 | GGGGTACCCACAGAATCATGAGGGACAGTGGGCTGTTTGGACAGTACCTCCTCACACCAG | 1980 | |
| QUERY: | 2764 | CCCGAGAGTCACTGTTCCTCATCAATGGGAGACAAAACACGCTCCGGTGTGAGGTGTCAG | 2823 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 1981 | CCCGAGAGTCACTGTTCCTCATCAATGGGAGACAAAACACGCTGCGGTGTGAGGTGTCAG | 2040 | |
| QUERY: | 2824 | GTATAAANNNNNNNACCACAGTGGTGTGGGTGGGTGAGGTATGAAGGGCCCAGAGCAGAG | 2883 | |
| | | \|\|\|\|\|\|\| \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 2041 | GTATAAAGGGGGGGACCACAGTGGTGTGGGTGGGTGAGGTATGAAGGGCCCAGAGCAGAG | 2100 | |
| QUERY: | 2884 | CCCTGGGCCAAGGAACACCCCCTAGTCCTGACACTGCAGCCTCAAGCAGGTACGCTGTAC | 2943 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 2101 | CCCTGGGCCAAGGAACACCCCCTAGTCCTGACACTGCAGCCTCAAGCAGGTACGCTGTAC | 2160 | |
| QUERY: | 2944 | ATTTTTACAGACAAAAGCAAAAACCTGTACTCGCTTTGTGGTTCAACACTGGTCTCCTTG | 3003 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 2161 | ATTTTTACAGACAAAAGCAAAAACCTGTACTCGCTTTGTGGTTCAACACTGGTCTCCTTG | 2220 | |
| QUERY: | 3004 | CAAGTTTCCTAGTATAAGGTATGCGCTGCTACCAAGATTGGGGTTTTTTCGTTAGGAAGT | 3063 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 2221 | CAAGTTTCCTAGTATAAGGTATGCGCTGCTACCAAGATTGGGGTTTTTTCGTTAGGAAGT | 2280 | |
| QUERY: | 3064 | ATGATTTATGCCTTGAGCTACGATGAGAACATATGCTGCTGTGTAAAGGGATCATTTCTG | 3123 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 2281 | ATGATTTATGCCTTGAGCTACGATGAGAACATATGCTGCTGTGTAAAGGGATCATTTCTG | 2340 | |
| QUERY: | 3124 | TGCCAAGCTGCACACCGAGTGACCTGGGGACATCATGGAACCAAGGGATCCTGCTCTCCA | 3183 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 2341 | TGCCAAGCTGCACACCGAGTGACCTGGGGACATCATGGAACCAAGGGATCCTGCTCTCCA | 2400 | |
| QUERY: | 3184 | AGCAGACACCTCTGTCAGTTGCCTTCACATAGTCATTGTCCCTTACTGCCAGACCCAGCC | 3243 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 2401 | AGCAGACACCTCTGTCAGTTGCCTTCACATAGTCATTGTCCCTTACTGCCAGACCCAGCC | 2460 | |
| QUERY: | 3244 | AGACTTTGCCCTGACGGAGTGGCCCGGAAGCAGAGGCCGACCAGGAGCAGGGGCCTCCCT | 3303 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 2461 | AGACTTTGCCCTGACGGAGTGGCCCGGAAGCAGAGGCCGACCAGGAGCAGGGGCCTCCCT | 2520 | |
| QUERY: | 3304 | CCCGAACTGAAAGCCCATCCGTCCTCGCGTGGGACCGCATCTTCTCCCTCGCAGCTGCTT | 3363 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 2521 | CCCGAACTGAAAGCCCATCCGTCCTCGCGTGGGACCGCATCTTCTCCCTCGCAGCTGCTT | 2580 | |
| QUERY: | 3364 | CTTGCTTTTCTTTCCATTTGACTTGCTGTAAGCCTGAGGGAGAGCCAACAAGACTTACTG | 3423 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 2581 | CTTGCTTTTCTTTCCATTTGACTTGCTGTAAGCCTGAGGGAGAGCCAACAAGACTTACTG | 2640 | |
| QUERY: | 3424 | CATCTTGGGGGATGGGGAAATCACTCACTTTATTTTGGAAATTTTTGATTNNNNNNNNNT | 3483 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| \| | | |
| SBJCT: | 2641 | CATCTTGGGGGATGGGGAAATCACTCACTTTATTTTGGAAATTTTTGATTAAAAAAAAAT | 2700 | |
| QUERY: | 3484 | TTTATAATCTCAAATGCTAGTAAGCAGAAAGATGCTCTCCGAGGTCCAACTATATCCTTC | 3543 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 2701 | TTTATAATCTCAAATGCTAGTAAGCAGAAAGATGCTCTCCGAGGTCCAACTATATCCTTC | 2760 | |
| QUERY: | 3544 | CCTGCCTTAGGCCGAGTCTCGGGGGTGGTCACAACCCCACATCCCACAGCCAGAAAGAAC | 3603 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 2761 | CCTGCCTTAGGCCGAGTCTCGGGGGTGGTCACAACCCCACATCCCACAGCCAGAAAGAAC | 2820 | |
| QUERY: | 3604 | AATGGTCATCTGAGAATACTGGCCCTGTCGACTATTGCCACCCTGCTTCTCCAAGAGCAG | 3663 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 2821 | AATGGTCATCTGAGAATACTGGCCCTGTCGACTATTGCCACCCTGCTTCTCCAAGAGCAG | 2880 | |
| QUERY: | 3664 | ACCAGGCCACCTCATCCGTAAGGACTCGGTTCTGTGTTGGGACCCCAAAAAACCAGAACA | 3723 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 2881 | ACCAGGCCACCTCATCCGTAAGGACTCGGTTCTGTGTTGGGACCCCAAAAAACCAGAACA | 2940 | |
| QUERY: | 3724 | AGTTCTGTGTGCCTCCTTTCAGCACAGAAGGGAGACATCTCATTAGTCAGGTCTGGTACC | 3783 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 2941 | AGTTCTGTGTGCCTCCTTTCAGCACAGAAGGGAGACATCTCATTAGTCAGGTCTGGTACC | 3000 | |
| QUERY: | 3784 | CCAGATTCAGGGCAGACTGGGCTTGCCTGGCAAGGTATGGGTGGCCTCCAGGCTCAATGC | 3843 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 3001 | CCAGATTCAGGGCAGACTGGGCTTGCCTGGCAAGGTATGGGTGGCCTCCAGGCTCAATGC | 3060 | |
| QUERY: | 3844 | AGAAACCCCAAGGACACGAGTGGGGCCAGGTGAGTTCCTGAAGCTATACCTTTTCAAAAC | 3903 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 3061 | AGAAACCCCAAGGACACGAGTGGGGCCAGGTGAGTTCCTGAAGCTATACCTTTTCAAAAC | 3120 | |
| QUERY: | 3904 | AGATTTTGTTTTCCTACCTGTGGCCCATCCACTCCTCTCTGGTACCCCATCCCCGCATCA | 3963 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 3121 | AGATTTTGTTTTCCTACCTGTGGCCCATCCACTCCTCTCTGGTACCCCATCCCCGCATCA | 3180 | |
| QUERY: | 3964 | GCACTGCAGAGAGAACACATTTCGGCGAGGGTTTTCTTACCCACATTCCCCAATCAATAC | 4023 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 3181 | GCACTGCAGAGAGAACACATTTCGGCGAGGGTTTTCTTACCCACATTCCCCAATCAATAC | 3240 | |
| QUERY: | 4024 | ACACACACTGCAGAACCCAGAACAGAAGGCCACAGGCTGGCACTACTGCATTCTCCTTAT | 4083 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 3241 | ACACACACTGCAGAACCCAGAACAGAAGGCCACAGGCTGGCACTACTGCATTCTCCTTAT | 3300 | |
| QUERY: | 4084 | GTGTCTCAGGCTGTGGTGACTCTCACATGGGCATCGAAGAAGTACAACCCACATAGCCCT | 4143 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 3301 | GTGTCTCAGGCTGTGGTGACTCTCACATGGGCATCGAAGAAGTACAACCCACATAGCCCT | 3360 | |
| QUERY: | 4144 | CTGGAGACCGCCTAGATCAGAGACTCAGCAAAAACAGGCTCGCCTTCCCTCTCCCACATA | 4203 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 3361 | CTGGAGACCGCCTAGATCAGAGACTCAGCAAAAACAGGCTCGCCTTCCCTCTCCCACATA | 3420 | |
| QUERY: | 4204 | TGAGTGGAACTTACATGTGTCCTGGTTTGAATGATCATTTTGCAAGCCACACGGGTTGGG | 4263 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 3421 | TGAGTGGAACTTACATGTGTCCTGGTTTGAATGATCATTTTGCAAGCCACACGGGTTGGG | 3480 | |
| QUERY: | 4264 | AGAGGTGGTCTCACCACAGACGTCTTTGCTAATTTGGCCACCTTCACCTACTGACATGAC | 4323 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 3481 | AGAGGTGGTCTCACCACAGACGTCTTTGCTAATTTGGCCACCTTCACCTACTGACATGAC | 3540 | |
| QUERY: | 4324 | CAGGATTTTCCTTTGCCATTAAGGAATGAACTCTTTCAAGGAGAGGAAACCCTAGACTCT | 4383 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 3541 | CAGGATTTTCCTTTGCCATTAAGGAATGAACTCTTTCAAGGAGAGGAAACCCTAGACTCT | 3600 | |
| QUERY: | 4384 | GTGTCACTCTCAACACACACAGCTCCTTTCACTCCTGCCTGACTGCCAAGCCACCTGCAT | 4443 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 3601 | GTGTCACTCTCAACACACACAGCTCCTTTCACTCCTGCCTGACTGCCAAGCCACCTGCAT | 3660 | |
| QUERY: | 4444 | CCCCCGCCCCAGATCTCATGAGATCAATCACTTGTATGTCTCACGCAACTTGGTCCACCA | 4503 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 3661 | CCCCCGCCCCAGATCTCATGAGATCAATCACTTGTATGTCTCACGCAACTTGGTCCACCA | 3720 | |
| QUERY: | 4504 | AACGCCTGTCCCCTGTAACTCCTAGGGGTGCGCCTAGACAGGTACGTCTGTTTTTTATTT | 4563 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 3721 | AACGCCTGTCCCCTGTAACTCCTAGGGGTGCGCCTAGACAGGTACGTCTGTTTTTTATTT | 3780 | |
| QUERY: | 4564 | TAAAAGATATGCTATGTAGATATAAGTTGAGGAAGCTCACCTCAAAAGCCTAGAATGCAG | 4623 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 3781 | TAAAAGATATQCTATGTAGATATAAGTTGAGGAAGCTCACCTCAAAAGCCTAGAATGCAG | 3840 | |
| QUERY: | 4624 | TTTCACAGTAGCTGGGATGCATGGATGACCCATCTCACCCCNNNNNNNNNCCTGCCTCAA | 4683 | |
| | | \|\|\|\|\|\|\|\|\|\|\|\|\|\|\|\|\|\|\|\|\|\|\|\|\|\|\|\|\|\|\|\|\|\|\|\|\|\|\|\|\| \|\|\|\|\|\|\|\|\|\| | | |
| SBJCT: | 3841 | TTTCACAGTAGCTGGGATGCATGGATGACCCATCTCACCCCTTTTTTTTTCCTGCCTCAA | 3900 | |
| QUERY: | 4684 | TATCTTGATATGTTATGTTTACTCCCAATCTCCCATTTTTACCACTAAAATTCTCCAACT | 4743 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 3901 | TATCTTGATATGTTATGTTTACTCCCAATCTCCCATTTTTACCACTAAAATTCTCCAACT | 3960 | |
| QUERY: | 4744 | TTCATAAACNNNNNNNNGGAAAAATTTCCATTGTATCAGCCCCTGACAGAAAAAGGATCT | 4803 | |
| | | \|\|\|\|\|\|\|\|\| \|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 3961 | TTCATAAACTTTTTTTTGGAAAAATTTCCATTGTATCAGCCCCTGACAGAAAAAGCATCT | 4020 | |
| QUERY: | 4804 | CTGAGCCTAAAGGAGGAAAAGTCCCACCAACTACCAGACCAGAACACGAGCCCCTCTGGG | 4863 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 4021 | CTGAGCCTAAAGGAGGAAAAGTCCCACCAACTACCAGACCAGAACACGAGCCCCTCTGGG | 4080 | |
| QUERY: | 4864 | CAGCAGGATTCCTAAGTCAAAGACCAGTTTGACCCAAACTGGCCTTTTAAAATAATCAGG | 4923 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 4081 | CAGCAGGATTCCTAAGTCAAAGACCAGTTTGACCCAAACTGGCCTTTTAAAATAATCAGG | 4140 | |
| QUERY: | 4924 | AGTGACAGAGTCAACTTCTGCAGCACCTGCTTCTCCCCCACTGTCCCTTCCATCTTGGAA | 4983 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 4141 | AGTGACAGAGTCAACTTCTGCAaCACCTGCTTCTCCCCCACTGTCCCTTCCATCTTGGAA | 4200 | |
| QUERY: | 4984 | TGTGTCTAAAAAAGCATAGCTGCCCTTTGCTGTCCTCAGAGTGCATTTCCTGGAGACGGC | 5043 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 4201 | TGTGTCTAAAAAAGCATAGCTGCCCTTTGCTGTCCTCAGAGTGCATTTCCTGGAGACGGC | 4260 | |
| QUERY: | 5044 | AGGCTTAGGTCTCACTGACAGCATGCCAGACACAACTGAATCGAAGCAGGCCTGAAGCCT | 5103 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 4261 | AGGCTTAGGTCTCACTGACAGCATGCCAGACACAACTGAATCGAAGCAGGCCTGAAGCCT | 4320 | |
| QUERY: | 5104 | AGGTCAGGGTTTCAGGAGTCCAGCCCCAGGAGGCAAAGTCACCAATGCAGGGACGTAAAT | 5163 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 4321 | AGGTCAGGGTTTCAGGAGTCCAGCCCCAGGAGGCAAAGTCACCAATGCAGGGAGGTAAAT | 4380 | |
| QUERY: | 5164 | GCCTTTTGGCAGGAAAACCAATAGAGTTGGTTGGGTGGGGAGTCAGGGGTGGGAGGAGAA | 5223 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 4381 | GCCTTTTGGCAGGAAAACCAATAGAGTTGGTTGGGTGGGGAGTCAGGGGTGGGAGGAGAA | 4440 | |
| QUERY: | 5224 | GGAGGAAGAGGAGGAAGGCCAGACTGGCCTGCCCTTTCTCCCATACTTCACCCCAGCAGA | 5283 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 4441 | GGAGGAAGAGGAGGAAGGCCAGACTGGCCTGCCCTTTCTCCCATACTTCACCCCAGCAGA | 4500 | |
| QUERY: | 5284 | GGTTCATGGGACACAGTTGGAAAGCCACTGGGAGGAAATGCCTCACTACAGGGGGGCCTC | 5343 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 4501 | GGTTCATGGGACACAGTTGGAAAGCCACTGGGAGGAAATGCCTCACTACAGGGGGGCCTC | 4560 | |
| QUERY: | 5344 | CTGTAGCAAGCCCAGCCGGTAATCCTCCTAATGAACCCACAAGGTCAATTCACAACTGAT | 5403 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 4561 | CTGTAGCAAGCCCAGCCGGTAATCCTCCTAATGAACCCACAAGGTCAATTCACAACTGAT | 4620 | |
| QUERY: | 5404 | ATCTTAGCTATTAAAGAAGTACTGACTTTACCAAAAGAATCATCAAGAAAGCTATTTATA | 5463 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 4621 | ATCTTAGCTATTAAAGAAGTACTGACTTTACCAAAAGAATCATCAAGAAAGCTATTTATA | 4680 | |
| QUERY: | 5464 | TAAACCCCCTCAGTCATTTTGAAATAAAATTAATTTTAC 5502 | | |
| | | \|\|\|\|\|\|\|\|\|\|\|\|\|\|\|\|\|\|\|\|\|\|\|\|\|\|\|\|\|\|\|\|\|\|\|\|\|\|\| | | |
| SBJCT: | 4681 | TAAACCCCCTCAGTCATTTTGAAATAAAATTAATTTTAC 4719 | | |

The FCTR2 amino acid sequence has 473 of 810 amino acid residues (58%) identical to, and 616 of 810 residues (76%) positive with, the 850 amino acid residue proteins from Homo sapiens KIAA1263 Protein fragment (ptnr: TREMBLNEW-ACC:BAA86577) (SEQ ID NO:47) (Table 2D).

TABLE 2D

BLASTP of FCTR2 against Homo sapiens KIAA1263 Protein fragment (SEQ ID NO:47)
ptnr: TREMBLNEW-ACC:BAA86577 KIAA1263 PROTEIN - Homo sapiens (Human), 850 aa
(fragment)

Length = 850
Score = 2573 (905.7 bits), Expect = 2.0e-267, P = 2.0e-267
Identities = 473/810 (58%), Positives = 616/810 (76%)

| | | | |
|---|---|---|---|
| QUERY: | 10 | LFRLSLKRALSSCPDLFGLSSRNELLASCGKKFCSRGSRCVLSRKTGEPECQCLEACRPS | 69 |
| | | \| \|\| \| + \| ++ \|\| \|+\| \| \|\| \|\|+\|\|+ \|\| \|++ \|+ | |
| SBJCT: | 40 | LMRLRHKEKNQESSRVKGFMIQDGPFGSCENKYCGLGRHCVTSRETGQAECACMDLCKRH | 99 |
| QUERY: | 70 | YVPVCGSDGRFYENHCKLHRAACLLGKRITVIHSKDCFLKGDTCTMAGYARLKNVLLALQ | 129 |
| | | \| \|\|\|\|\|\|\| \|\|\|\|\|\|++\|\|\|\|\|\| ++\|\|++\|++\|\|\| \|\|\| \| \|+++\|\|+\|\| \|\| | |
| SBJCT: | 100 | YKPVCGSDGEFYENHCEVHRAACLKKQKITIVHNEDCFFKGDKCKTTEYSKMKNMLLDLQ | 159 |
| QUERY: | 130 | TRLQPLQEGDSRQ-DPASQKRLLVESLFRDLDADGNGHLSSSELAQHVLKKQDLDEDLLG | 188 |
| | | + +\|\| ++ \| \|+\|+\|\|\|+ +\|+ \|\|\| \|\| + +\|\| \| \|+\|+++\| +\|\| | |
| SBJCT: | 160 | NQKYIMQENENPNGDDISRKKLLVDQMFKYFDADSNGLVDINELTQ-VIKQEELGKDLPD | 218 |
| QUERY: | 189 | CSPGDLLRFDDYNSDSSLTLREFYMAFQVVQLSLAPEDRVSVTTVTVGLSTVLTCAVHGD | 248 |
| | | \|+ \|\|++\|\|+\|+\| \| \| \|\|\| \|\|\|\|+\|\|\|\| + + \| \| \|\|\| \| \|\| \|\|+ \| | |
| SBJCT: | 219 | CTLYVLLKYDDFNADKHLALEEFYRAFQVIQLSLPEDQKLSITAATVGQSAVLSCAIQGT | 278 |
| QUERY: | 249 | LRPPIIWKRNGLTLNFLDLEDINDFGEDDSLYITKVTTIHMGNYTCHASGHEQLFQTHVL | 308 |
| | | \|\|\|\|\|\|\|\|\|\| + \|\| \|\|\|\|\|\|\|\|\|\|+\| \|\|\|\|\|\|\|\|\| \|+\|\|\|\|\|+\| \|+\|\|++\|\|\|+ | |
| SBJCT: | 279 | LRPPIIWKRNNIILNNLDLEDINDFGDDGSLYITKVTTTHVGNYTCYADGYEQVYQTHIF | 338 |
| QUERY: | 309 | QVNVPPVIRVYPESQAQEPGVAASLRCHAEGIPMPRITWLKNGVDVSTQMSKQLSLLANG | 368 |
| | | \|\|\|\|\|\|\|\|\|\|\|\|\|\|\|\|+\|\|\|\| \|\|\|\|\|\|\|\|\|\|\| \|++ \|\|\|\|\|+\|++ ++\|\|\|\|+\| \|\|\| | |
| SBJCT: | 339 | QVNVPPVIRVYPESQAREPGVTASLRCHAEGIPKPQLGWLKNGIDITPKLSKQLTLQANG | 398 |
| QUERY: | 369 | SELHISSVRYEDTGAYTCIAKNEVGVDEDISSLFIEDSARKTLANILWREEGLSVGNMFY | 428 |
| | | \|\|+\|\|\|+\|\|\|\|\|\|\|\|\|\|\|\|\|\|\|\| \|\|\|\|\|\|\|\|\|\|+\|\|\|\|\|\|\|\|\|\|\|\|\|\|\|\|\|\| +\|\|\|\|\| | |
| SBJCT: | 399 | SEVHISNVRYEDTGAYTCIAKNEAGVDEDISSLFVEDSARKTLANILWREEGLGIGNMFY | 458 |
| QUERY: | 429 | VFSDDGIIVIHPVDCEIQRHLKPTEKIFMSYEEICPQREKNATQPCQWVSAVNVRNRYIY | 488 |
| | | \|\| +\|\|\| \|\| \|++\|\| \|\|\|+\|\|+\|\|+ +\|+\|\|+ \| + \| \| \| \|\|\|\|\|++++\|\| | |
| SBJCT: | 459 | VFYEDGIKVIQPIECEFQRHIKPSEKLLGFQDEVCPKAEGDEVQRCVWASAVNVKDKFIY | 518 |
| QUERY: | 489 | VAQPALSRVLVVDIQAQKVLQSIGVDPLPAKLSYDKSHDQVWVLSWGDVHKSRPSLQVIT | 548 |
| | | \|\|\|\| \| \|\|\|+\|\|+\|+\|\|\|+\|++ \|\|+\| \|\| \|\|\|\|\|\|\|\|\|\|\|\|\|\| + \|+ \|+\|\|\|\|\| | |
| SBJCT: | 519 | VAQPTLDRVLIVDVQSQKVVQAVSTDPVPVKLHYDKSHDQVWVLSWGTLEKTSPTLQVIT | 578 |
| QUERY: | 549 | EASTGQSQHLIRT-----PFAGVDDFFIPPTNLIINHIRFGFIFNKSDPAVHKVDLETMM | 603 |
| | | \|\| \| \| \| \| \|\|\|\|\|\|\| \| \|\|\| \|+\|\|\|\|\| +\| + \|+ \|+\|\|\|\|\| | |
| SBJCT: | 579 | LASGNVPHHTIHTQPVGKQFDRVDDFFIPTTTLIITHMRFGFILHKDEAALQKIDLETMS | 638 |
| QUERY: | 604 | PLKTIGLHHHGCVPQAMAHTHLGGYFFIQCRQDSPASAARQLLVDSVTDSVLGPNGDVTG | 663 |
| | | +\|\|\| \| + \|\|\|\|++\|+\|\|\|\|\|\|+\|\| \|+ \|\| + + \|++\|\| \|\|\|\|\|+\| \| \|\|\|\| | |
| SBJCT: | 639 | YIKTINLKDYKCVPQSLAYTHLGGYYFIGCKPDSTGAVSPQVMVDGVTDSVIGFNSDVTG | 698 |
| QUERY: | 664 | TPHTSPDGRFIVSAAADSPWLHVQEITVRGEIQTLYDLQINSGISDLAFQRSFTESNQYN | 723 |
| | | \|\|+ \|\|\|\| ++\|\| + \|\| \|\|+\|\|\|\|\| +\|+ \| \|\|\|\|\|\|\| \|\|\|\|++\|\|\| | |
| SBJCT: | 699 | TPYVSPDGHYLVSINDVKGLVRVQYITIRGEIQEAFDIYTNLHISDLAFQPSFTEAHQYN | 758 |
| QUERY: | 724 | IYAALHTEPDLLFLELSTGKVGMLKNLKEPPAGPAQPWGGTHRIMRDSGLFGQYLLTPAR | 783 |
| | | \|\| + \|+ \|+\|\|+\|\|\|+\|\|\| \|+\|+\|\|\|\| \|\| +\| ++\|\|\|\|\|\|\|\|\|+\|\|++ | |
| SBJCT: | 739 | IYGSSSTQTDVLFVELSSGKVKMIKSLKEPLKAEEWPWNRKNRQIQDSGLFGQYLMTPSK | 818 |
| QUERY: | 784 | ESLFLINGRQNTLRCEVSGIKGGTTVVWVGE | 814 |
| | | +\|\|\|+++\|\| \| \| \|\|++ ++ \| \|\|+\|\|\|+ | |
| SBJCT: | 819 | DSLFILDGRLNKLNCEITEVEKGNTVIWVGD | 849 |

Amino acids 123-815 of FCTR2 also have 693 of 693 amino acid residues (100%) identical to the 693 amino acid residue protein fragment of KIAA 1061 Protein from Homo sapiens (ptnr: TREMBLNEW-ACC: BAA83013) (SEQ ID NO:48) (Table 2E).

TABLE 2E
BLASTP of FCTR2 against KIAA1061 Protein [Fragment] (SEQ ID NO:48)
ptnr:TREMBLNEW-ACC:BAA83013 KIAA1O62 PROTEIN - Homo sapiens (Human),
693 aa (fragment).

Length = 693
Score = 3623 (1275.4 bits), Expect = 0.0, P = 0.0
Identities = 693/693 (100%), Positives = 693/693 (100%)

| | | | |
|---|---|---|---|
| QUERY: | 123 | NVLLALQTRLQPLQEGDSRQDPASQKRLLVESLFRDLDADGNGHLSSSELAQHVLKKQDL | 182 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 1 | NVLLALQTRLQPLQEGDSRQDPASQKRLLVESLFRDLDADGNGHLSSSELAQHVLKKQDL | 60 |
| QUERY: | 183 | DEDLLGCSPGDLLRFDDYNSDSSLTLREFYMAFQVVQLSLAPEDRVSVTTVTVGLSTVLT | 242 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 61 | DEDLLGCSPGDLLRFDDYNSDSSLTLREFYMAFQVVQLSLAPEDRVSVTTVTVGLSTVLT | 120 |
| QUERY: | 243 | CAVHGDLRPPIIWKRNGLTLNFLDLEDINDFGEDDSLYITKVTTIHMGNYTCHASGHEQL | 302 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 121 | CAVHGDLRPPIIWKRNGLTLNFLDLEDINDFGEDDSLYITKVTTIHMGNYTCHASGHEQL | 180 |
| QUERY: | 303 | FQTHVLQVNVPPVIRVYPESQAQEPGVAASLRCHAEGIPMPRITWLKNGVDVSTQMSKQL | 362 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 181 | FQTHVLQVNVPPVIRVYPESQAQEPGVAASLRCHAEGIPMPRITWLKNGVDVSTQMSKQL | 240 |
| QUERY: | 363 | SLLANGSELHISSVRYEDTGAYTCIAKNEVGVDEDISSLFIEDSARKTLANILWREEGLS | 422 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 241 | SLLANGSELHISSVRYEDTGAYTCIAKNEVGVDEDISSLFIEDSARKTLANILWREEGLS | 300 |
| QUERY: | 423 | VGNMFYVFSDDGIIVIHPVDCEIQRHLKPTEKIFMSYEEICPQREKNATQPCQWVSAVNV | 482 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 301 | VGNMFYVFSDDGIIVIHPVDCEIQRHLKPTEKIFMSYEEICPQREKNATQPCQWVSAVNV | 360 |
| QUERY: | 483 | RNRYIYVAQPALSRVLVVDIQAQKVLQSIGVDPLPAKLSYDKSHDQVWVLSWGDVHKSRP | 542 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 361 | RNRYIYVAQPALSRVLVVDIQAQKVLQSIGVDPLPAKLSYDKSHDQVWVLSWGDVHKSRP | 420 |
| QUERY: | 543 | SLQVITEASTGQSQELIRTPFAGVDDFFIPPTNLIINHIRFGFIFNKSDPAVHKVDLETM | 602 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 421 | SLQVITEASTGQSQHLIRTPFAGVDDFFIPPTNLIINHIRFGFIFNKSDPAVHKVDLETM | 480 |
| QUERY: | 603 | MPLKTIGLHHHGCVPQAMAHTHLGGYFFIQCRQDSPASAARQLLVDSVTDSVLGPNGDVT | 662 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 481 | MPLKTIGLHEHGCVPQAMAHTHLGGYFFIQCRQDSPASAARQLLVDSVTDSVLGPNGDVT | 540 |
| QUERY: | 663 | GTPHTSPDGRFIVSAAADSPWLHVQEITVRGEIQTLYDLQINSGISDLAFQRSFTESNQY | 722 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 541 | GTPHTSPDGRFIVSAAADSPWLHVQEITVRGEIQTLYDLQINSGISDLAFQRSFTESNQY | 600 |
| QUERY: | 723 | NIYAALHTEPDLLFLELSTGKVGMLKNLKEPPAGPAQPWGGTHRIMRDSGLFGQYLLTPA | 782 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 601 | NIYAALHTEPDLLFLELSTGKVGMLKNLKEPPAGPAQPWGGTHRIMRDSGLFGQYLLTPA | 660 |
| QUERY: | 783 | RESLFLINGRQNTLRCEVSGIKGGTTVVWVGEV | 815 |
| | | \|\|\|\|\|\|\|\|\|\|\|\|\|\|\|\|\|\|\|\|\|\|\|\|\|\|\|\|\|\|\|\|\| | |
| SBJCT: | 661 | RESLFLINGRQNTLRCEVSGIEGGTTVVWVGEV | 693 |

The amino acid sequence of the FCTR2 protein has 451 of 772 amino acid residues (58%) identical to, and 586 of 772 residues (75%) positive with, the 773 amino acid residue proteins hypothetical protein DKFZp566D234.1 from Homo sapiens (fragments) (ptnr: SPTREMBL-ACC: CAB70877.1) (SEQ ID NO:49) (Table 2F).

TABLE 2F
BLASTP of FCTR2 against hypothetical protein DKFZp566D234.1 (SEQ ID NO:49)
>GI|11360192|PIR||T46283 HYPOTHETICAL PROTEIN DKFZP566D234.1 - HUMAN (FRAGMENTS)
GI|6808053|EMB|CAB70877.1| (AL137695) HYPOTHETICAL PROTEIN [HOMO SAPIENS]

LENGTH = 773
SCORE = 911 BITS (2354), EXPECT = 0.0
IDENTITIES = 451/772 (58%), POSITIVES = 586/772 (75%), GAPS = 7/772 (0%)

| | | | |
|---|---|---|---|
| QUERY: | 49 | CVLSRKTGEPECQCLEACRPSYVPVCGSDGRFYENHCKLHRAACLLGKRITVIHSKDCFL | 108 |
| | | \|\| \|\|+\|\|+ \|\| \|++ \|+ \| \|\|\|\|\|\|\| \|\|\|\|\|\|++\|\|\|\|\|\| ++\|\|++\|++\|\|\| | |
| SBJCT: | 2 | CVTSRETGQAECACMDLCKRHYKPVCGSDGEFYENHCEVHRAACLKKQKITIVHNEDCFF | 61 |
| QUERY: | 109 | KGDTCTMAGYARLKNVLLALQTRLQPLQEGDSRQ-DPASQKRLLVESLFRDLDADGNGHL | 167 |
| | | \|\|\| \| +++\|\|+\|\| \|\| + +\|\| ++ \| \|+\|+\|\|\|+ +\|+ \|\|\| \| + | |
| SBJCT: | 62 | KGDKCKTTECSKMKNMLLDLQNQRYIMQENENPNGDDISRKKLLVDQMFKYFDADSNDLV | 121 |
| QUERY: | 168 | SSSELAQHVLKKQDLDEDLLGCSPGDLLRFDDYNSDSSLTLREFYMAFQVVQLSLAPEDR | 227 |
| | | +\|\| \| \|+\|+++\| +\|\| \|+ \|\|++\|\|+\|+\| \| \| \|\|\| \|\|\|\|+\|\|\|\| + + | |
| SBJCT: | 122 | DINELTQ-VIKQEELGKDLFDCTLYVLLKYDDFNADKHLALEEFYRAFQVIQLSLPEDQK | 180 |
| QUERY: | 228 | XXXXXXXXXXXXXXXCAVHGDLRPPIIWKRNGLTLNFLDLEDINDFGEDDSLYITKVTTI | 287 |
| | | \|\|+ \| \|\|\|\|\|\|\|\|\|\| + \|\| \| \|\|\|\|\|\|\|\|+\| \|\|\|\|\|\|\|\|\| | |
| SBJCT: | 181 | LSITAATVGQSAVLSCAIQGTLRPPIIWKRNNIILNNLGLEDINDFGDDGSLYITKVTTT | 240 |
| QUERY: | 288 | HMGNYTCHASGHEQLFQTHVLQVNVPPVIRVYPESQAQEPGVAASLRCHAEGIPMPRITW | 347 |
| | | \|+\|\|\|\|\|+\| \|+\|\|++\|\|\|+ \|\|\|\|\|\|\|\|\|\|\|\|\|\|\|\|+\|\|\|\| \|\|\|\|\|\|\|\|\|\|\| \|++ \| | |
| SBJCT: | 241 | HVGNYTCYADGYEQVYQTHIFQVNVPPVIRVYPESQAREPGVTASLRCHAEGIPKPQLGW | 300 |
| QUERY: | 348 | LKNGVDVSTQMSKQLSLLANGSELHISSVRYEDTGAYTCIAKNEVGVDEDISSLFIEDSA | 407 |
| | | \|\|\|\|+\|++ ++\|\|\|\|+\| \|\|\|\|\|+\|\|\|+\|\|\|\|\|\|\|\|\|\|\|\|\|\|\|\| \|\|\|\|\|\|\|\|\|\|+\|\|\|\| | |
| SBJCT: | 301 | LKNGIDITPKLSKQLTLQANGSEVHISNVRYEDTGAYTCIAKNEAGVDEDISSLFVEDSA | 360 |
| QUERY: | 408 | RKTLANILWREEGLSVGNMFYVFSDDGIIVIHPVDCEIQRHLKPTEKIFMSYEEICPQRE | 467 |
| | | \|\|\|\|\|\|\|\|\|\|\|\|\|\| +\|\|\|\|\|\|\| +\|\|\| \|\| \|++\|\| \|\|\|+\|\|+\|\|+ +\|+\|\| \| | |
| SBJCT: | 361 | RKTLANILWREEGLGIGNMFYVFYEDGIKVIQPIECEFQRHIKPSEKLLGFQDEVCPIAE | 420 |
| QUERY: | 468 | KNATQPCQWVSAVNVRNRYIYVAQPALSRVLVVDIQAQKVLQSIGVDPLPAKLSYDKSHD | 527 |
| | | + \| \| \| \|\|\|\|\|++++\|\|\|\|\|\| \| \|\|\|+\|\|+\|+\|\|\|+\|++ \|\|+\| \|\| \|\|\|\|\|\| | |
| SBJCT: | 421 | GDEVQRCVWASAVNVKDKFIYVAQPTLDRVLIVDVQSQKVVQAVSTDPVPVKLHYDKSHD | 480 |
| QUERY: | 528 | QVWVLSWGDVHKSRPSLQVITEASTGQSQHLIRT-----PFAGVDDFFIPPTNLIINHIR | 582 |
| | | \|\|\|\|\|\|\|\| + \|+ \|+\|\|\|\|\| \|\| \| \| \| \| \|\|\|\|\|\|\| \| \|\|\| \|+\| | |
| SBJCT: | 481 | QVWVLSWGTLEKTSPTLQVITLASGNVPHHTIHTQPVGKQFDRVDDFFIPTTTLIITHMR | 540 |
| QUERY: | 583 | FGFIFNKSDPAVHKVDLETMMPLKTIGLHHHGCVPQAMAHTHLGGYFFIQCRQDSPASAA | 642 |
| | | \|\|\|\| +\| + \|+ \|+\|\|\|\|\| +\|\|\| \| + \|\|\|\|++\|+\|\|\|\|\|\|+\|\| \|+ \|\| + + | |
| SBJCT: | 541 | FGFILHKDEAALQKIDLETMSYIKTINLKDYKCVPQSLAYTHLGGYYFIGCKPDSTGAVS | 600 |
| QUERY: | 643 | RQLLVDSVTDSVLGPNGDVTGTPHTSPDGRFIVSAAADSPWLHVQEITVRGEIQTLYDLQ | 702 |
| | | \|++\|\| \|\|\|\|\|+\| \| \|\|\|\|\|\|+ \|\|\|\| ++\|\| + \|\| \|\|+\|\|\|\|\| +\|+ | |
| SBJCT: | 601 | PQVMVDGVTDSVIGFNSDVTGTPYVSPDGHYLVSINDVKGLVRVQYITIRGEIQEAFDIY | 660 |
| QUERY: | 703 | INSGISDLAFQRSFTESNQYNIYAALHTEPDLLFLELSTGKVGMLKNLKEPPAGPAQPWG | 762 |
| | | \| \|\|\|\|\|\|\| \|\|\|\|++\|\|\|\|\| + \|+ \|+\|\|+\|\|\|+\|\|\| \|+\|+\|\|\|\| \|\| | |
| SBJCT: | 661 | TNLHISDLAFQPSFTEAHQYNIYGSSSTQTDVLFVELSSGKVKMIKSLKEPLKAEEWPWN | 720 |
| QUERY: | 763 | GTHRIMRDSGLFGQYLLTPARESLFLINGRQNTLRCEVSGIKGGTTVVWVGE | 814 |
| | | +\| ++\|\|\|\|\|\|\|\|\|+\|\|+++\|\|\|+++\|\| \| \| \|\|++ ++ \| \|\|+\|\|\|+ | |
| SBJCT: | 721 | RKNRQIQDSGLFGQYLMTPSKDSLFILDGRLNKLNCEITEVEKGNTVIWVGD | 772 |

The amino acid sequence of the FCTR2 protein has 61 of 194 amino acid residues (31%) identical to, and 90 of 194 residues (45%) positive with, the 306 amino acid residue protein Follastin-Related Protein 1 Precursor from Rattus Norvegicus (ptnr: GenBank Acc:Q62632) (SEQ ID NO:50) (Table 2G).

TABLE 2G
BLASTP of FCTR2 against Follastatin-Related Protein 1 Precursor from
Rattus Norvegicus (SEQ ID NO:50)

```
>GI|2498392|SP|Q62632|FRP RAT FOLLISTATIN-RELATED PROTEIN 1 PRECURSOR
  GI|1083669|PIR||S51361 FOLLISTATIN-RELATED PROTEIN PRECURSOR - RAT
  GI|536900|GB|AAA66063.1|(U06864) FOLLISTATIN-RELATED PROTEIN PRECURSOR [RATTUS
NORVEGICUS]
LENGTH = 306
SCORE = 86.4 BITS (213), EXPECT = 1E-15
IDENTITIES = 61/194 (31%), POSITIVES = 90/194 (45%), GAPS = 26/194 (13%)
```

| | | | |
|---|---|---|---|
| QUERY: | 38 | CGKKFCSRGSRCVLSRKTGEPECQCLEACRPSYVPVCGSDGRFYENHCKLERAACLLGKR | 97 |
| | | \| \|\| \| \| ++ \| \|\|\| \| \|+\| \|+\| \|\|\|\|\|+\|+ \| \|\|\|+\|\|\| \|\|\| \| + | |
| SBJCT: | 29 | CANVFCGAGRECAVTEK-GEPTCLCIEQCKPHKRPVCGSNGKTYLNHCELERDACLTGSK | 87 |
| QUERY: | 98 | ITVIHSKDCFLKGD--------TCTMAGYARLKNVLLA-LQTRLQPLQEGDSRQDPASQK | 148 |
| | | \| \| + \| \| \| \| \|+ ++ \|+ + \| \| \| | |
| SBJCT: | 88 | IQVDYDGHCKEKKSVSPSASPVVCYQANRDELRRRIIQWLEAEIIP----DGWFSKGSNY | 143 |
| QUERY: | 149 | RLLVESLFRDLDADGNGHLSSSELAQHVLK-----------KQDLDEDLLGCSPGDLLRF | 197 |
| | | +++ \|+ \| +\|+ \|\| \|\|\| + \| + \|+ ++ \| \| \|+ | |
| SBJCT: | 144 | SEILDKYFKSFD-NGDSHLDSSEFLKFVEQNETAVNITAYPNQENNKLLRGLCVDALIEL | 202 |
| QUERY: | 198 | DDYNSDSSLTLREF | 211 |
| | | \| \|+\| \|+ +\|\| | |
| SBJCT: | 203 | SDENADWKLSFQEF | 216 |

The amino acid sequence of the FCTR2 protein has 61 of 194 amino acid residues (31%) identical to, and 89 of 194 residues (45%) positive with, the 306 amino acid residue protein Follastin-Related Protein 1 Precursor from Mus musculus (GenBank Acc:Q62356) (SEQ ID NO:51) (Table 2H).

TABLE 2H
BLASTP of FCTR2 against Follastatin-Related Protein 1 Precursor from
Mus musculus (SEQ ID NO:51)

```
>GI|6679871|REF|NP 032073.1|FOLLISTATIN-LIKE [MUS MUSCULUS]
  GI|2498391|SP|Q62356|FRP MOUSE FOLLISTATIN-RELATED PROTEIN 1 PRECURSOR (TGF-BETA-
INDUCIBLE PROTEIN
TSC-36)
  GI|481186|PIR||S38251 FOLLISTATIN-RELATED PROTEIN - MOUSE
  GI|349006|GB|AAC37633.1 (M91380) TGF-BETA-INDUCIBLE PROTEIN [MUS MUSCULUS]
LENGTH = 306
SCORE = 85.2 BITS (210), EXPECT = 3E-15
IDENTITIES = 61/194 (31%), POSITIVES = 89/194 (45%), GAPS = 26/194 (13%)
```

| | | | |
|---|---|---|---|
| QUERY: | 38 | CGKKFCSRGSRCVLSRKTGEPECQCLEACRPSYVPVCGSDGRFYENHCKLHRAACLLGKR | 97 |
| | | \| \|\| \| \| ++ \| \|\|\| \| \|+\| \|+\| \|\|\|\|\|+\|+ \| \|\|\|+\|\|\| \|\|\| \| + | |
| SBJCT: | 29 | CANVFCGAGRECAVTEK-GEPTCLCIEQCKPHKRPVGCSNGKTYLNHCELHRDACLTGSK | 87 |
| QUERY: | 98 | ITVIHSKDCFLKGDT--------CTMAGYARLKNVLLA-LQTRLQPLQEGDSRQDPASQK | 148 |
| | | \| \| + \| \| \| \| \|+ \|+ \|+ + \| \| \| | |
| SBJCT: | 88 | IQVDYDGHCKEKKSASPSASPVVCYQANRDELRRRLIQWLEAEIIP----DGWFSKGSNY | 143 |
| QUERY: | 149 | RLLVESLFRDLDADGNGHLSSSELAQHVLKK-----------QDLDEDLLGCSPGDLLRF | 197 |
| | | +++ \|+ \| +\|+ \|\| \|\|\| + \| + \|+ ++ \| \|+ | |
| SBJCT: | 144 | SEILDKYFKSFD-NGDSHLDSSEFLKFVEQNETAINITTYADQENNKLLRSLCVDALIEL | 202 |
| QUERY: | 198 | DDYNSDSSLTLREF | 211 |
| | | \| \|+\| \|+ +\|\| | |
| SBJCT: | 203 | SDENADWKLSFQEF | 216 |

The amino acid sequence of the FCTR2 protein has 63 of 193 amino acid residues (32%) identical to, and 89 of 193 residues (45%) positive with, the 299 amino acid residue protein Follastatin-Related Protein from the African Clawed Frog (GenBank Acc:JGO187) (SEQ ID NO:52) (Table 21).

TABLE 21
BLASTP of FCTR2 against Follastatin-Related Protein from the African
Clawed Frog (SEQ ID NO:52)

>GI|7512162||PIR|JG0187 FOLLISTATIN-RELATED PROTEIN - AFRICAN CLAWED FROG
LENGTH = 299
SCORE = 81.8 BITS (201), EXPECT = 3E-14
IDENTITIES = 63/193 (32%), POSITIVES = 89/193 (45%), GAPS = 25/193 (12%)

| | | | |
|---|---|---|---|
| QUERY: | 38 | CGKKFCSRGSRCVLSRKTGEPECQCLEACRPSYVPVCGSDGRFYENHCKLHRAACLLGKR | 97 |
| | | \| \|\| \| \| ++ \| \|+\| \| \|+\| \|+ \|\|\|\|\|+\|+ \| \|\|\|+\|\|\| \|\|\| \| + | |
| SBJCT: | 28 | CANVFCGAGRECAVTEK-GDPTCDCIEKCKSHKRPVCGSNGKTYLNHCELHRDACLTGSK | 86 |
| QUERY: | 98 | ITVIHSKDCFLK-GDT-------CTMAGYARL-KNVLLALQTRLQPLQEGDSRQDPASQK | 148 |
| | | \| \| + \| \| \|\| \| + + + \|+ \|\|\| + \| \| \| | |
| SBJCT: | 87 | IQVDYDGHCKEKTSDTPAAVPVACYQSDRDEMRRRVIHWLQTEITP----DGWFSKGSDY | 148 |
| QUERY: | 149 | RLLVESLFRDLDADGNGHLSSSELAQHVLKKQDL------DED----LLGCSPGDLLRFD | 198 |
| | | +++ \|+ \| \|\|+ \|\| \|+\|\| + + \| \|\|+ \| \|+ | |
| SBJCT: | 143 | SEILDRYFKKFD-DGDSHLDSAELQSFLEQSQSTNITTYKDEETNRMLKSLCVEALIELS | 201 |
| QUERY: | 199 | DYNSDSSLTLREF | 211 |
| | | \| \|+\| \| \|\| | |
| SBJCT: | 202 | DENADWKLNKNEF | 214 |

The amino acid sequence of the FCTR2 protein has 59 of 194 amino acid residues (30%) identical to, and 90 of 194 residues (45%) positive with, the 308 amino acid residue protein Follistatin-Related Protein 1 Precursor from Homo sapiens (GenBank Acc:Q12841) (SEQ ID NO:53) (Table 2J).

TABLE 2J
BLASTP of FCTR2 against Follistatin-Related Protein 1 Precursor from
Homo sapiens (SEQ ID NO:53)

>GI|5901956|REF|NP 009016.1|FOLLISTATIN-LIKE 1 [HOMO SAPIENS]
GI|2498390|SP|Q12841|FRP HUMAN FOLLISTATIN-RELATED PROTEIN 1 PRECURSOR
GI|1082372|PIR||S51362 FOLLISTATIN-RELATED PROTEIN - HUMAN
GI|536898|GB|AAA66062.1|(U06863) FOLLISTATIN-RELATED PROTEIN PRECURSOR [HOMO SAPIENS]
GI|3184393|DBJ|BAA28707.1|(D89937) FOLLISTATIN-RELATED PROTEIN (FRP) [HOMO SAPIENS]
GI|12652619|GB|AAH00055.1|AAH00055 (BC000055) FOLLISTATIN-LIKE 1 [HOMO SAPIENS]
LENGTH = 308
SCORE = 82.9 BITS (204), EXPECT = 1E-14
IDENTITIES = 59/194 (30%), POSITIVES = 90/194 (45%), GAPS 26/194 (13%)

| | | | |
|---|---|---|---|
| QUERY: | 38 | CGKKFCSRGSRCVLSRKTGEPECQCLEACRPSYVPVCGSDGRFYENHCKLHRAACLLGKR | 97 |
| | | \| \|\| \| \| ++ \| \|\|\| \| \|+\| \|+\| \|\|\|\|\|+\|+ \| \|\|\|+\|\|\| \|\|\| \| + | |
| SBJCT: | 31 | CANVFCGAGRECAVTEK-GEPTCLCIEQCKPHVRPVCGSNGKTYLNHCELHRDACLTGSK | 89 |
| QUERY: | 98 | ITVIHSKDCFLKGD--------TCTMAGYARLKNVLLA-LQTRLQPLQEGDSRQDPASQK | 148 |
| | | \| \| + \| \| \| + \|+ ++ \|+ + \| \| \| | |
| SBJCT: | 90 | IQVDYDGHCKEKKSVSPSASPVVCYQSNRDELRRRIIQWLEAEIIP----DGWFSKGSNY | 145 |
| QUERY: | 149 | RLLVESLFRDLDADGNGHLSSSELAQHVLKK-----------QDLDEDLLGCSPGDLLRF | 197 |
| | | +++ \|++ \| +\|+ \| \|\|\| + \| + \|+ ++ \| \| \|+ | |
| SBJCT: | 146 | SEILDKYFKNFD-NGDSRLDSSEFLKFVEQNETAINITTYPDQENNKLLRGLCVDALIEL | 204 |
| QUERY: | 198 | DDYNSDSSLTLREF | 211 |
| | | \| \|+\| \|+ +\|\| | |
| SBJCT: | 205 | SDENADWKLSFQEF | 218 |

The amino acid sequence of the FCTR2 protein has 35 of 69 amino acid residues (50%) identical to, and 45 of 69 residues (64%) positive with, the 315 amino acid residue Flik protein [Gallus gallus] (EMBL Acc:CAB42968.1) (SEQ ID NO:54) (Table 2K).

TABLE 2K
BLASTP of FCTR2 against Flik protein [Gallus gallus](SEQ ID NO:54)

>GI|4837645|EMB|CAB42968.1|(AJ238977) FLIK PROTEIN [GALLUS GALLUS]
LENGTH = 315
SCORE = 79.8 BITS (196), EXPECT = 1E-13
IDENTITIES = 35/69 (50%), POSITIVES = 45/69 (64%), GAPS = 1/69 (1%)

| | | | |
|---|---|---|---|
| QUERY: | 38 | CGKKFCSRGSRCVLSRKTGEPECQCLEACRPSYVPVCGSDGRFYENHCKLHRAACLLGKR | 97 |
| | | \| \|\| \|\|+ \| ++ \| \|\|\| \| \|+\| \|+\| \|\|\|\|\|+\|+ \| \|\|\|+\|\|\| \|\|\| \| + | |
| SBJCT: | 31 | CANVFCGRGAECAVTEK-GEPTCLCIEQCKPHGRPVCGSNGKTYLNHCELHRDACLTGSK | 89 |
| QUERY: | 98 | ITVIHSKDC | 106 |
| | | \|\|++\| | |
| SBJCT: | 90 | IQVDYDGHC | 98 |

The amino acid sequence of the FCTR2 protein has 49 of 152 amino acid residues (32%) identical to, and 65 of 152 residues (42%) positive with a 272-420 amino acid fragment and, 31 of 83 residues (37%) identical to and 44 of 83 residues (52%) positive with a 248-329 amino acid fragment, both of the 1375 amino acid residue Frazzled gene protein [Drosophila melanogaster] (GenBankAcc:T13822) (SEQ ID NO:55) (Table 2L).

TABLE 2L
BLASTP of FCTR2 against Frazzled gene protein [Drosophila melanogaster]
(SEQ ID NO:55)

>GI|75118611|PIR||T-13822 FRAZZLED GENE PROTEIN - FRUIT FLY (DROSOPHILA MELANOGASTER)
GI|1621115|GB|AAC47314.1|(U71001) FRAZZLED [DROSOPHILA MELANOGASTER]
LENGTH = 1375
SCORE = 69.4 BITS (169), EXPECT = 2E-10
IDENTITIES = 49/152 (32%), POSITIVES = 65/152 (42%), GAPS = 4/152 (2%)

| | | | |
|---|---|---|---|
| QUERY: | 243 | CAVHGDLRPPIIWKRNGLTLNFLDLEDINDFGEDDSLYITKVTTIHMGNYTCHASGH-EQ | 301 |
| | | \| +\| +\| \| \| \|\|\|+ \|+\| \|\|+ \|\| \|+ \| \|\|\| \| \|\| + | |
| SBJCT: | 272 | CVANGVPKPQIKWLRNGMDLDFNDLDSRFSIVGTGSLQISSAEDIDSGNYQCRASNTVDS | 331 |
| QUERY: | 302 | LFQTHVLQVNVPPVIRVYPESQAQEPGVAASLRCHAEGIPMPRITWLKNGVDVSTQMSKQ | 361 |
| | | \| +\|\| \|\| \|+ \|+\| \| \| \| \| \|\|\|\|\| ++ \| | |
| SBJCT: | 332 | LDAQATVQVQEPPKFIKAPKDTTAHEXDEPELKCDIWGKPKPVIRWLKNGDLITPNDYMQ | 391 |
| QUERY: | 362 | LSLLANGSELHISSVRYEDTGAYTCIAKNEVG | 393 |
| | | \| +\| \| \| + \| \| + \|+ \| \| | |
| SBJCT: | 392 | ---LVDGHNLKILGLLNSDAGMFQCVGTNAAG | 420 |

SCORE = 52.9 BITS (126), EXPECT = lE-05
IDENTITIES = 31/83 (37%), POSITIVES = 44/83 (52%), GAPS = 2/83 (2%)

| | | | |
|---|---|---|---|
| QUERY: | 311 | NVPPVIRVYPESQAQEPGVAASLRCHAEGIPMPRITWLKNGVDVS-TQMSKQLSLLANGS | 369 |
| | | +\| \| \| \| + \| +\| \| \| \|+\| \|+\| \|\|+\|\|+\|+ + + \|++ \|\| | |
| SBJCT: | 248 | SVAPSFLVGPSPKTVREGDTVTLDCVANGVPKPQIKWLRNGMDLDFNDLDSRFSIVGTGS | 307 |
| QUERY: | 370 | ELHISSVRYEDTGAYTCIAKNEV | 392 |
| | | \| \|\|\| \|+\| \| \| \| \| \| | |
| SBJCT: | 308 | -LQISSAEDIDSGNYQCRASNTV | 329 |

The amino acid sequence of the FCTR2 protein has 53 of 177 amino acid residues (29%) identical to, and 78 of 177 residues (43%) positive with a 366-539 amino acid fragment, 51 of 170 residues (30%) identical to and 74 of 170 residues (43%) positive with a 276-438 amino acid fragment, 46 of 165 amino acid residues (27%) identical to, and 74 of 165 amino acid residues positive with a 185-341 amino acid fragment, 48 of 167 amino acid residues (28%) identical to and 70 of 167 amino acid residues (41%) positive with a 77-243 amino acid fragment, and 28 of 84 amino acid residues (33%) and 37 of 84 amino acid residues positive with a 56-139 amino acid fragment all of the protein 1395 residue Roundabout 1 protein [Drosophila melanogaster] (GenBankAcc:AAC38849.1) (SEQ ID NO:56) (Table 2M).

Table 2M
BLASTP of FCTR2 against Roundabout 1 protein [Drosophila melanogaster]
(SEQ ID NO:56)

>GI|2804782|GB|AAC38849.1|(AF040989) ROUNDABOUT 1 [DROSOPHILA MELANOGASTER]
LENGTH = 1395
SCORE = 69.8 BITS (170), EXPECT = lE-lO
IDENTITIES = 53/177 (29%), POSITIVES = 78/177 (43%), GAPS = 11/177 (6%)

| | | | |
|---|---|---|---|
| QUERY: | 243 | CAVHGDLRPPIIWKRNGL-TLNFLDLEDINDF-GEDDSLYITKVTTIHMGNYTCHA---- | 296 |
| | | \| \|+ \| + \| + \|+ \|\| \| +\|\|\|\|\|\|+ \| +\| \|\| \| \| \| \| | |
| SBJCT: | 366 | CMASGNPPPSVFWTKEGVSTLMFPNSSHGRQYVAADGTLQITDVRQEDEGYYVCSAFSVV | 425 |
| QUERY: | 297 | --SGHEQLFQTHVLQVNVPPVIRVYPESQAQEPGVAASLRCHAEGIPMPRITWLKNGVDV | 354 |
| | | \| \| + \|\|+\|++ \| +\| \| \|+\| \| \| \| \| \|\|\| \| +\| \| | |
| SBJCT: | 426 | DSSTVRVFLQVSSVDERPPPIIQIGPANQTLPKGSVATLPCRATGNPSPRIKWFHDGHAV | 485 |
| QUERY: | 355 | STQMSKQLSLLANGSELHISSVRYEDTGAYTCIAKNEVGVDEDISSLFIEDSARKTL | 411 |
| | | \| + \|++ \|\| \| + ++ \|+\| \|\|\| \| \| \| ++\| +\| +\| | |
| SBJCT: | 486 | --QAGNRYSII-QGSSLRVDDLQLSDSGTYTCTASGERGETSWAATLTVEKPGSTSL | 539 |

SCORE = 56.3 BITS (135), EXPECT = 1E-06
IDENTITIES = 51/170 (30%), POSITIVES = 74/170 (43%), GAPS = 12/170 (7%)

| | | | |
|---|---|---|---|
| QUERY: | 243 | CAVHGDLRPPIIWKRNGLTLNFLDLEDINDFGEDDSLYITKVTTIHMGNYTCHASGH-EQ | 301 |
| | | \|+\| \|\| \| ++\|\|+ + ++\| + \|\| \|+ +\| \| \| \| \| + \| | |
| SBJCT: | 276 | CSVGGDPPPKVLWKKEEGNIPVSRARILHD---EKSLEISNITPTDEGTYVCEAHNNVGQ | 332 |
| QUERY: | 302 | LFQTHVLQVNVPPVIRVYPESQAQEPGVAASLRCHAEGIPMPRITWLKNGVDVSTQM--- | 358 |
| | | + \| \|+ \|\| \| ++ \| \| \| \| \| \| + \| \| \| \|\|\| \| | |
| SBJCT: | 333 | ISARASLIVHAPPNFTKRPSNKKVGLNGVVQLPCMASGNPPPSVFWTKEG--VSTLMFPN | 390 |
| QUERY: | 359 | -SKQLSLLANGSELHISSVRYEDTGAYTCIAKNEVGVDEDISSLFIEDSA | 407 |
| | | \| +\| \| \|+ \|\| \|\| \| \| \| \| + \|\| +\|++ \|+ | |
| SBJCT: | 391 | SSHGRQYVAADGTLQITDVRQEDEGYYVCSAFSV--VDSSTVRVFLQVSS | 438 |

SCORE = 51.7 BITS (123), EXPECT = 3E-05
IDENTITIES = 46/165 (27%), POSITIVES = 74/165 (43%), GAPS = 20/165 (12%)

| | | | |
|---|---|---|---|
| QUERY: | 251 | PPIIWKRNGLTINFLDLEDINDFG--------EDDSLYITKVTTIHMGNYTCHASG---- | 298 |
| | | \| +\|\| ++\|+ \|+ \|\|+ ++ \|\| + +\| \|+ \| \| \|\|\| \| \| | |
| SBJCT: | 185 | PTLIWIKDGVPLD--DLKAMS-FGASSRVRIVDGGNLLISNVEPIDEGNYKCIAQNLVGT | 241 |
| QUERY: | 299 | HEQLFQTHVLQVNVPPVIRVYPESQAQEPGVAASLRCHAEGIPMPRITWLKNGVDVSTQM | 358 |
| | | \| + ++\|\| \| \|+ \| \| \|+ \| \| \| \|++ \| \| ++ | |
| SBJCT: | 242 | RESSYAKLIVQVK--PYFMKEPKDQVMLYGQTATFHCSVGGDPPPKVLWKKEEGNIPVSR | 299 |
| QUERY: | 359 | SKQLSLIANGSELHISSVRYEDTGAYTCIAKNEVGVDEDISSLFI | 403 |
| | | ++ +\| + \| \|\|++ \| \| \| \| \| \| \|\| +\|\| + | |
| SBJCT: | 300 | AR---ILHDEKSLEISNITPTDEGTYVCEAHNNVGQISARASLIV | 341 |

SCORE = 44.0 BITS (103), EXPECT = 0.007
IDENTITIES = 48/167 (28%), POSITIVES = 70/167 (41%), GAPS = 13/167 (7%)

| | | | |
|---|---|---|---|
| QUERY: | 243 | CAVHGDLRPPIIWKRNGLTLNFLDLEDINDFGEDDSLYITKVTTIHM----GNYTCHASG | 298 |
| | | \| \| \| \| \| \| ++\| ++ + + +\| +\|+ + \| \| \| \| | |
| SBJCT: | 77 | CKVEGKPEPTIEWFKDGEPVSTNEKKSHRVQFKDGALFFYRTMQGKKEQDGGEYWCVAKN | 136 |
| QUERY: | 299 | H-EQLFQTHV-LQVNV-PPVIRVYPESQAQEPGVAASLRCH-AEGIPMPRITWLKNGVDV | 354 |
| | | \| \| \|\|+ \| \|\| \|+ \| \| \| \| +\|\|\| \| + \|+\|+\|\| + | |
| SBJCT: | 137 | RVGQAVSRHASLQIAVLRDDFRVEPKDTRVAKGETALLECGPPKGIPEPTLIWIKDGVPL | 196 |
| QUERY: | 355 | STQMSKQLSL-----LANGSELHISSVRYEDTGAYTCIAKNEVGVDE | 396 |
| | | + + +\| \| \|\|+\| \| \| \| \|\|\|+\| \|\| \| | |
| SBJCT: | 197 | DDLKAMSFGASSRVRIVDGGNLLISNVEPIDEGNYKCIAQNLVGTRE | 243 |

SCORE = 42.9 BITS (100), EXPECT = 0.014
IDENTITIES = 28/84 (33%), POSITIVES = 37/84 (43%), GAPS = 4/84 (4%)

| | | | |
|---|---|---|---|
| QUERY: | 314 | PVIRVYPESQAQEPGVAASLRCHAEGIPMPRITWLKNGVDVSTQMSKQLSLLANGSELH- | 372 |
| | | \| \| +\| + \|+\| \| \|\| \| \| \| \| \|+\| \|\|\| \| + \| | |
| SBJCT: | 56 | PRIIEHPTDLVVKKNEPATLNCKVEGKPEPTIEWFKDGEPVSTNEKKSHRVQFKDGALFF | 115 |
| QUERY: | 373 | ---ISSVRYEDTGAYTCIAKNEVG | 393 |
| | | + + +\| \| \| \|+\|\|\| \|\| | |
| SBJCT: | 116 | YRTMQGKKEQDGGEYWCVAKNRVG | 139 |

The amino acid sequence of the FCTR2 protein has 55 of 157 amino acid residues (35%) identical to, and 75 of 157 residues (47%) positive with a 620-775 amino acid fragment, 49 of 163 residues (30%) identical to and 71 of 163 residues (43%) positive with a 335-492 amino acid fragment, 32 of 85 amino acid residues (37%) identical to, and 48 of 85 amino acid residues (55%) positive with a 1305-1388 amino acid fragment, 37 of 143 amino acid residues (25%) identical to and 60 of 143 amino acid residues (41%) positive with a 183-319 amino acid fragment, 43 of 174 amino acid residues (24%) and 70 of 174 amino acid residues (39%) positive with a 711-884 amino acid fragment, and 46 of 165 residues (27%) identical to and 69 of 165 residues positive with a 831-884 amino acid fragment all of the protein 1395 residue Down Syndrome Cell Adhesion Molecule Precursor (CHD2) from Homo Sapiens (GenBankAcc:060469) (SEQ ID NO:57) (Table 2N).

TABLE 2N
BLASTP of FCTR2 against Down Syndrome Cell Adhesion Molecule Precursor

```
>gi|12643619|sp|O60469|DSCA Human DOWN SYNDROME CELL ADHESION MOLECULE PRECURSOR (CHD2)
  GI|67400131|GB|AAF27525.1|AF217525 1 (AF217525) DOWN SYNDROME CELL ADHESION
MOLECULE [HOMO SAPIENS]
                    LENGTH = 2012
  SCORE = 70.6 BITS (172), EXPECT = 6E−11
  IDENTITIES = 55/157 (35%), POSITIVES = 75/157 (47%), GAPS = 7/157 (4%)
```

| | | | | |
|---|---|---|---|---|
| QUERY: | 245 | VHGDLRPPIIWKRNGLTLNFLDLEDINDFGEDDSLYITKVTTIHMGNYTCHASGHEQLFQ | 304 | (SEQ ID NO:57) |
| | | \| \|\|\| \| \|+++\| + \|++ \|\| \|+ ++ +\| \|\|\|\|\| \| + | | |
| SBJCT: | 620 | VSGDLPITITWQKDGRPIPGSLGVTIDNIDFTSSLRISNLSLMHNGNYTCIARNEAAAVE | 679 | |
| QUERY: | 305 | THV-LQVNVPPVIRVYPESQAQEPGVAASLRCHAEGIPMPRITW-LKNGVDVST----QM | 358 | |
| | | \| \| \|\|\| \| \| \| \| \| \| \| \|\|\| \|+\| \| \| \| \| + | | |
| SBJCT: | 680 | HQSQLIVRVPPKFVVQPRDQDGIYGKAVILNCSAEGYPVPTIVWKFSKGAGVPQFQPIAL | 739 | |
| QUERY: | 359 | SKQLSLLANGSELHISSVRYEDTGAYTCIAKNEVGVD 395 | | |
| | | + ++ +\|+\|\|\| \| \| \| \|\|+\| \| \| \|+\|\| \| | | |
| SBJCT: | 740 | NGRIQVLSNGS-LLIKHVVEEDSGYYLCKVSNDVGAD 775 | | |

```
  SCORE = 50.6 BITS (120), EXPECT = 7E−05
  IDENTITIES = 49/163 (30%), POSITIVES = 71/163 (43%), GAPS = 16/163 (9%)
```

| | | | |
|---|---|---|---|
| QUERY: | 243 | CAVHGDLRPPIIWKRNGLTLNFLDLEDINDFGEDDSLYITKVTTIHMGNYTCHASGHEQL | 302 |
| | | \|+\| \| + \| \|\|\| \|\| \| ++ \| + + \| \| \| + | |
| SBJCT: | 335 | CSVTGTEDQELSWYRNGEILNPGKNVRITGINHEN-LIMDHMVKSDGGAYQCFVRKDKLS | 393 |
| QUERY: | 303 | FQTH---VLQVNVPPVIRVYPESQAQEPGVAASLRCHAEGIPMPRITW-------LKNGV | 352 |
| | | \| + \|\|+ \| +\| + \| + \| \|\| \|+ +\| \|+\| \|\|\| \|\| \| | |
| SBJCT: | 394 | AQDYVQVVLEDGTPKIISAFSE-KVVSPAEPVSLMCNVKGTPLPTITWTLDDDPILKGG- | 451 |
| QUERY: | 353 | DVSTQMSKQLSLLAN-GSELHISSVRYEDTGAYTCIAKNEVGV 394 | |
| | | \| ++\|+ ++ \| \| \|+\|\|\| + \| \| \| \| \| \| \|\| | |
| SBJCT: | 452 | --SHRISQMITSEGNVVSYLNISSSQVRDGGVYRCTANNSAGV 492 | |

```
  SCORE = 47.9 BITS (113), EXPECT = 5E−04
  IDENTITIES = 32/85 (37%), POSITIVES = 48/85 (55%), GAPS = 6/85 (7%)
```

| | | | |
|---|---|---|---|
| QUERY: | 333 | LRCHAEGIPMPRITWLK--NGVDVSTQMSKQLSLLANGSELHISSVRYEDTGAYTCIAKN | 390 |
| | | \| \| \| \| \| \| + \|+\| \|\| + + \|+ +\|\|\| + \| +\|+ \|\|+\| \|+\|\|\| \| | |
| SBJCT: | 1305 | LPCKAVGDPSPAVKWMKDSNGTPSLVTIDGRRSIFSNGSFI-IRTVKAEDSGYYSCIANN | 1363 |
| QUERY: | 391 | EVGVDEDISSLFIE---DSARKTLA 412 | |
| | | \| \|\| \| +\| ++ \| \| \|++ | |
| SBJCT: | 1364 | NWGSDEIILNLQVQVPPDQPRLTVS 1388 | |

```
  SCORE = 42.9 BITS (100), EXPECT = 0.015
  IDENTITIES = 37/143 (25%), POSITIVES = 60/143 (41%), GAPS = 6/143 (4%)
```

| | | | |
|---|---|---|---|
| QUERY: | 270 | INDFGEDDSLYITKVTTIHMGNYTCHASGHEQLFQTHVLQVNVPPVIRVYPESQAQEPGV | 329 |
| | | \| \| +\| \|\| + \| \| \| +\|\| + \| \| \| + + \| | |
| SBJCT: | 183 | IKDVQNEDGLYNYRCITRHRYTGETRQSNSARLFVSD--PANSAPSILDGFDHRKAMAGQ | 240 |
| QUERY: | 330 | AASLRCHAEGIPMPRITWLKNGVDVSTQMSKQLSLLANGSELHISSVRYEDTGAYTCIAK | 389 |
| | | \| \| \| \| \| \| \|\|\|+ ++ ++\| + \| \| \| ++\| \|+\|+\| \| | |
| SBJCT: | 241 | RVELPCKALGHPEPDYRWLKD--NMPLELSGRFQKTVTG--LLIENIRPSDSGSYVCEVS | 296 |
| QUERY: | 390 | NEVGVDEDISSLFIEDSARKTLA 412 | |
| | | \| \| + \| \|+++ + \|++ | |
| SBJCT: | 297 | NRYGTAKVIGRLYVKQPLKATIS 319 | |

```
  SCORE = 41.3 BITS (96), EXPECT = 0.047
  IDENTITIES = 43/174 (24%), POSITIVES = 70/174 (39%), GAPS = 11/174 (6%)
```

| | | | |
|---|---|---|---|
| QUERY: | 243 | CAVHGDLRPPIIWK--RNGLTLNF--LDLEDINDFGEDDSLYITKVTTIHMGNYTCHASG | 298 |
| | | \|+ \| \| \|+\|\| + \| + \| + \|\| \| \| \| \| \| \| | |
| SBJCT: | 711 | CSAEGYPVPTIVWKFSKGAGVPQFQPIALNGRIQVLSNGSLLIKHVVEEDSGYYLCKVSN | 770 |
| QUERY: | 299 | H--EQLFQTHVLQVNVPPVIRVYPESQAQEPGVAASLRCHAEGIPMPRITWLKNGVDVST | 356 |
| | | + ++ \| \| +\| +\| \|\| + \| + \| \| \| + \| \| ++ | |
| SBJCT: | 771 | DVGADVSKSMYLTVKIPAMITSYPNTTLATQGQKKEMSCTAHGEKPIIVRWEKEDRIINP | 830 |
| QUERY: | 357 | QMSKQLSLLANGSELHISSVRY-----EDTGAYTCIAKNEVGVDEDISSLFIED 405 | |
| | | +\|++ \| \| \|\|+++ \|\|+\| ++\| \| \|\| \| \| \| \| +++ | |
| SBJCT: | 831 | EMARYLVSTKEVGEEVISTLQILPTVREDSGFFSCHAINSYGEDRGIIQLTVQE 884 | |

```
  SCORE = 40.6 BITS (94), EXPECT = 0.074
  IDENTITIES = 46/165 (27%), POSITIVES = 69/165 (40%), GAPS = 7/165 (4%)
```

| | | | |
|---|---|---|---|
| QUERY: | 243 | CAVHGDLRPPIIWKRNGLTLNFLDLEDINDFGEDDSLYITKVTT-IHMGNYTCHASGHEQ | 301 |
| | | \| \| \| \| \| +\| \| \| + \| + +\| ++ \| + \| \|\|\|+ \| | |
| SBJCT: | 525 | CRVIGYPYYSIKWYKNSNLLPFNHRQVA--FENNGTLKLSDVQKEVDEGEYTCNVLVQPQ | 582 |
| QUERY: | 302 | LFQTHVLQVN--VPPVIRVYPESQAQEPGVAASLRCHAEGIPMP-RITWLKNGVDVSTQM | 358 |
| | | \| + + \| \|\|\| \|+ + \| \| + \| +\| \|\|\| \|+\| + + | |
| SBJCT: | 583 | LSTSQSVHVTVKVPPFIQPF-EFPRFSIGQRVFIPCVVVSGDLPITITWQKDGRPIPGSL | 641 |
| QUERY: | 359 | SKQLSLLANGSELHISSVRYEDTGAYTCIAKNEVGVDEDISSLFI | 403 |
| | | + + \| \| \|\|++ \| \|\|\|\|\|+\|\| \| \| \| + | |
| SBJCT: | 642 | GVTIDNIDFTSSLRISNLSLMHNGNYTCIARNEAAAVEHQSQLIV | 686 |

The amino acid sequence of the FCTR2 protein has 55 of 194 amino acid residues (28%) identical to, and 86 of 194 residues (44%) positive with Limbic System-Associated Membrane Protein Precursor (LSAMP) from Homo sapiens (SWISSPROT Acc:Q13449) (SEQ ID NO:58) (Table 2O).

TABLE 2O
BLASTP of FCTR2 against Limbic System-Associated
Membrane Protein (SEQ ID NO:58) Precursor

PTNR:SWISSPROT-ACC:Q13449 LIMBIC SYSTEM-ASSOCIATED
MEMBRANE PROTEIN PRECURSOR (LSAMP) - HOMO SAPIENS
(HUMAN), 338 AA.

LENGTH = 338
SCORE = 191 (67.2 BITS),
EXPECT = 6.7E−12,
P = 7E−12
IDENTITIES = 55/194 (28%),
POSITIVES = 86/194 (44%)

The amino acid sequence of the FCTR2 protein has 68 of 190 amino acid residues (35%) identical to, and 92 of 190 residues (48%) positive with Putative Neuronal Cell Adhesion Molecule, Short Form from Mus musculus (SPTREMBL Acc:070246) (SEQ ID NO:59) (Table 2P).

TABLE 2P
BLASTP of FCTR2 against Putative Neuronal Cell
Adhesion Molecule, Short (SEQ ID NO:59) Form from
Mus musculus

PTNR:SPTREMBL-ACC:070246 PUTATIVE NEURONAL CELL
ADHESION MOLECULE (PUNC) (PUTATIVE NEURONAL CELL
ADHESION MOLECULE, SHORT FORM) - MUS MUSCULUS
(MOUSE), 793 AA

LENGTH = 793
SCORE = 203 (71.5 BITS),
EXPECT = 7.0E−12,
SUM P(2) = 7.0E−12
IDENTITIES = 68/190 (35%),
POSITIVES = 92/190 (48%)

The amino acid sequence of the FCTR2 protein has 58 of 199 amino acid residues (29%) identical to, and 91 of 199 residues (45%) positive with CHLAMP, G11-Isoform Precursor from Gallus gallus (SPTREMBL Acc: 002869) (SEQ ID NO:60) (Table 2Q).

TABLE 2Q
BLASTP of FCTR2 against CHLAMP,G11-Isoform
Precursor from Gallus gallus (SEQ ID NO:60)

PTNR:SPTREMBL-ACC:002869 CHLAMP, G11-ISOFORM
PRECURSOR - GALLUS GALLUS (CHICKEN), 350 AA.

LENGTH = 350
SCORE = 191 (67.2 BITS),
EXPECT = 7.7E−12,
P = 7.7E−12
IDENTITIES = 58/199 (29%),
POSITIVES = 91/199 (45%)

The amino acid sequence of the FCTR2 protein has 55 of 194 amino acid residues (28%) identical to, and 86 of 194 residues (44%) positive with Limbic System-Associated Membrane Protein Precursor (LSAMP) from Rattus norvegicus (SWISSPROT Acc:Q62813) (SEQ ID NO:61) (Table 2R).

TABLE 2R
BLASTP of FCTR2 against Limbic System-Associated
Membrane Protein Precursor (LSAMIP) from
Rattus norvegicus (SEQ ID NO:61)

PTNR:SWISSPROT-ACC:Q62813 LIMBIC SYSTEM-ASSOCIATED
MEMBRANE PROTEIN PRECURSOR (LSANP) - RATTUS
NORVEGICUS (RAT), 338 AA.

LENGTH = 338
SCORE = 188 (66.2 BITS),
EXPECT = 1.5E−11,
P = 1.5E−11
IDENTITIES = 55/194 (28%),
POSITIVES = 86/194 (44%)

FCTR2 protein has similarity to cell adhesion molecules, follistatin, roundabout and frazzled (see BlastP results). These genes are involved in neuronal development and reproductive physiology. Frazzled encodes a Drosophila member of the DCC immunoglobulin subfamily and is required for CNS and motor axon guidance (Cell 87:197-204(1996)). Characterization of a rat C6 glioma-secreted follistatin-related protein (FRP) and cloning and sequence of the human homologue is described in Eur. J. Biochem. 225:937-946(1994). This protein may modulate the action of some growth factors on cell proliferation and differentiation. FRP binds heparin. The follistatin-related protein is a secreted protein and has one follistatin-like domain. The cloning and early dorsal axial expression of Flik, a chick follistatin-related gene and evidence for involvement in dorsalization/neural induction is presented in Dev. Biol. 178:327-342(1996). Roundabout controls axon crossing of the CNS midline and defines a novel subfamily of evolutionarily conserved guidance receptors, as shown in Cell 92:205-215(1998). cDNA cloning and structural analysis of the human limbic-system-associated membrane protein (LAMP) is described in Gene 170:189-195(1996). LAMP, a protein of the OBCAM family that contains three immunoglobulin-like C2-type domains, mediates selective neuronal growth and axon targeting. LAMP contributes to the guidance of developing axons and remodeling of mature circuits in the limbic system. This protein is essential for normal growth of the hippocampal mossy fiber projection. LAMP is attached to the membrane by a GPI-Anchor. It is expressed on limbic neurons and fiber tracts as well as in single layers of the superior colliculus, spinal chord and cerbellum. Characterization of the human full-length PTK7 cDNA encoding a receptor protein tyrosine kinase-like molecule closely related to chick KLG is disclosed in J. Biochem. 119:235-239(1996). Based upon homology, FCTR2 proteins and each homologous protein or peptide may share at least some activity.

Functions and therapeutic uses:

The OMIM gene map has identified this region which the invention maps to (5q21-5q3 1) as associated with susceptibility to the following diseases (OMIM Ids are underlined):

Allergy and asthma

Hemangioma,

capillary infantile Schistosoma mansoni infection, susceptibility/resistance to Spinocerebellar ataxia

Bronchial asthma

Plasmodium falciparum parasitemia,

intensity of Corneal dystrophy, Groenouw type I, 121900; Corneal dystrophy,lattice type I, 122200;

Reis-Bucklers corneal dystrophy;Corneal dystrophy, Avellino type Eosinophilia, familial Myelodysplastic syndrome;

Myelogenous leukemia, Acute Cutis laxa, recessive, type I, Deafness, autosomal dominant nonsyndromic sensorineural, 1 Contractural arachnodactyly, Congenital Neonatal alloimmune thrombocytopenia;

Glycoprotein Ia deficiency Male infertility;

Charcot-Marie-Tooth neuropathy, Demyelinating Gardner syndrome;

Adenomatous polyposis coli;

Colorectal cancer;

Desmoid disease, hereditary, 135290;

Turcot syndrome,276300;

Adenomatous polyposis coli, attenuated

Colorectal cancer

Therefore the invention is implicated in at least all of the above mentioned diseases and may have therapeutic uses for these diseases.

This sequence has similarity to cell adhesion molecules, follistatin, roundabout and frazzled (see BlastP results). These genes are involved in neuronal development and reproductive physiology. Therefore the invention is also implicated in disorders such as or therapeutic uses for:

Neurodegenerative disorders, nerve trauma, epilepsy, mental health conditions

Tissue regeneration in vivo and in vitro

Female reproductive system disorders and pregnancy

FCTR3

FCTR3, is an amino acid type II membrane, neurestin-like protein. The FCTR3a nucleic acid of 1430 nucleotides (also designated 10129612.0.118) is shown in Table 3A. An ORF was identified beginning with an ATG initiation codon at nucleotides 69-71 and ending with a TAG codon at nucleotides 1212-1214. A putative untranslated region upstream from the initiation codon and downstream from the termination codon is underlined in Table 3A, and the start and stop codons are in bold letters.

TABLE 3A
FCTR3a Nucleotide Sequence

| | |
|---|---|
| AAAAAAGGCGGGGGGTGGACTTAGCAGTGTAATTTGAGACCGGTGGTAAGGATTGGAGCGAGCTAGAGATGCTGCACGCTGCTA | (SEQ ID NO:5) |
| ACAAGGGAAGGAAGCCTTCAGCTGAGGCAGGTCGTCCCATTCCACCTACATCCTCGCCTAGTCTCCTCCCATCTGCTCAGCTGC | |
| CTAGCTCCCATAATCCTCCACCAGTTAGCTGCCAGATGCCATTGCTAGACAGCAACACCTCCCATCAAATCATGGACACCAACC | |
| CTGATGAGGAATTCTCCCCCAATTCATACCTGCTCAGAGCATGCTCAGGGCCCCAGCAAGCCTCCAGCAGTGGCCCTCCGAACC | |
| ACCACAGCCAGTCGACTCTGAGGCCCCCTCTCCCACCCCCTCACAACCACACGCTGTCCCATCACCACTCGTCCGCCAACTCCC | |
| TCAACAGGAACTCACTGACCAATCGGCGGAGTCAGATCCACGCCCCGGCCCCAGCGCCCAATGACCTGGCCACCACACCAGAGT | |
| CCGTTCAGCTTCAGGACAGCTGGGTGCTAAACAGCAACGTGCCACTGGAGACCCGGCACTTCCTCTTCAAGACCTCCTCGGGGA | |
| GCACACCCTTGTTCAGCAGCTCTTCCCCGGGATACCCTTTGACCTCAGGAACGGTTTACACGCCCCCGCCCCGCCTGCTGCCCA | |
| GGAATACTTTCTCCAGGAAGGCTTTCAAGCTGAAGAAGCCCTCCAAATACTGCAGCTGGAAATGTGCTGCCCTCTCCGCCATTG | |
| CCGCGGCCCTCCTCTTGGCTATTTTGCTGGCGTATTTCATAGTGCCCTGGTCGTTGAAAAACAGCAGCATAGACAGTGGTGAAG | |
| CAGAAGTTGGTCGGCGGGTAACACAAGAAGTCCCACCAGGGGTGTTTTGGAGGTCACAAATTCACATCAGTCAGCCCCAGTTCT | |
| TAAAGTTCAACATCTCCCTCGGGAAGGACGCTCTCTTTGGTGTTTACATAAGAAGAGGACTTCCACCATCTCATGCCCAGTATG | |
| ACTTCATGGAACGTCTGGACGGGAAGGAGAAGTGGAGTGTGGTTGAGTCTCCCAGGGAACGCCGGAGCATACAGACCTTGGTTC | |
| AGAATGAAGCCGTGTTTGTGCAGTACCTGGATGTGGGCCTGTGGCATCTGGCCTTCTACAATGATGGAAAAGACAAAGAGATGG | |
| TTTCCTTCAATACTGTTGTCCTAGATGGGACCATCTAGTTGCAGAAAAACAAGCTCAGGGCGCCCACTGATTTGACATTATGAT | |
| TCAGTGCAGGACTGTCCACGTAACTGCCATGGGAATGGTGAATGTGTGTCCGGGGTGTGTCACTGTTTCCCAGGATTTCTAGGA | |
| GCAGACTGTGCTAAAGACCTTCCTGCCTTGACTTTCTGCAAGACAATCATTAATAAAGCTGCTCTGTAAATACTAAAAAAAAAA | |
| CA | |

The FCTR3 polypeptide (SEQ ID NO:5) encoded by SEQ ID NO:5 is 381 amino acid residues and is presented using the one-letter code in Table 3B.

TABLE 3B
Encoded FCTR3a protein sequence.

| | |
|---|---|
| MLHAANKGRKPSAEAGRPIPPTSSPSLLPSAQLPSSHNPPPVSCQMPLLDSNTSHQIMDTNPDEEFSPNSYLLRACSGPQQASS | (SEQ ID NO:6) |
| SGPPNHHSQSTLRPPLPPPHNHTLSHHHSSANSLNRNSLTNRRSQIHAPAPAPNDLATTPESVQLQDSWVLNSNVPLETRHFLF | |
| KTSSGSTPLFSSSSPGYPLTSGTVYTPPPRLLPRNTFSRKAFKLKKPSKYCSWKCAALSAIAAALLLAILLAYFIVPWSLKNSS | |
| IDSGEAEVGRRVTQEVPPGVFWRSQIHISQPQFLKFNISLGKDALFGVYIRRGLPPSHAQYDFMERLDGKEKWSVVESPRERRS | |
| IQTLVQNEAVFVQYLDVGLWHLAFYNDGKDKEMVSFNTVVLDGTI | |

In an alternative embodiment, the 5′ end of the FCTR3a nucleic acid could be extended as it is in the 9826 bp FCTR3b (also referred to herein as 10129612.0.405) shown in Table 3C. An ORF was identified beginning with an ATG initiation codon at nucleotides 280-282 and ending with a TAA codon at nucleotides 8479-8481. A putative untranslated region upstream from the initiation codon and downstream from the termination codon is underlined in Table 3C, and the start and stop codons are in bold letters. Italicized bases 1-201 refer to a variable 5′ region that will be further discussed below.

TABLE 3C
FCTR3b Nucleotide Sequence

| | |
|---|---|
| TTTAAATCCTCATACCTTAAAGGAGATGTGTATATAAGGGAGTTGGAACCAGCATTAGATGAGTTGACAAAAATGCACTT | (SEQ ID NO:7) |
| TCAGTTCTAGAGGTCTGGGAAGTCCAAGAACAAGGTGCTGGCAGATTGGATTCCCCGTGAGGGCTTTCTTCCTGGCTTGA | |
| AGTTGGCTGCTTTCCTGCTGAGACTTCTCATGGCAGAGACTGAGGGTGGCAAAGTGACAAGTGCCAAAACTCAGGCCTGA | |
| CTTTTCTGAAAACATCAGCATTCTGCCATATCTGGAATAATGGATGTAAAGGACCGGCGACACCGCTCTTTGACCAGAGG | |
| ACGCTGTGGCAAAGAGTGTCGCTACACAAGCTCCTCTCTGGACAGTGAGGACTGCCGGGTGCCCACACAGAAATCCTACA | |
| GCTCCAGTGAGACTCTGAAGGCCTATGACCATGACAGCAGGATGCACTATGGAAACCGAGTCACAGACCTCATCCACCGG | |
| GAGTCAGATGAGTTTCCTAGACAAGGAACCAACTTCACCCTTGCCGAACTGGGCATCTGTGAGCCCTCCCCACACCGAAG | |
| CGGCTACTGCTCCGACATGGGGATCCTTCACCAGGGCTACTCCCTTAGCACAGGGTCTGACGCCGACTCCGACACCGAGG | |
| GAGGGATGTCTCCAGAACACGCCATCAGACTGTGGGGCAGAGGGATAAAATCCAGGCGCAGTTCCGGCCTGTCCAGTCGT | |
| GAAAACTCGGCCCTTACCCTGACTGACTCTGACAACGAAAACAAATCAGATGATGAGAACGGTCGTCCCATTCCACCTAC | |
| ATCCTCGCCTAGTCTCCTCCCATCTGCTCAGCTGCCTAGCTCCCATAATCCTCCACCAGTTAGCTGCCAGATGCCATTGC | |
| TAGACAGCAACACCTCCCATCAAATCATGGACACCAACCCTGATGAGGAATTCTCCCCCAATTCATACCTGCTCAGAGCA | |
| TGCTCAGGGCCCCAGCAAGCCTCCAGCAGTGGCCCTCCGAACCACCACAGCCAGTCGACTCTGAGGCCCCCTCTCCCACC | |
| CCCTCACAACCACACGCTGTCCCATCACCACTCGTCCGCCAACTCCCTCAACAGGAACTCACTGACCAATCGGCGGAGTC | |
| AGATCCACGCCCCGGCCCCAGCGCCCAATGACCTGGCCACCACACCAGAGTCCGTTCAGCTTCAGGACAGCTGGGTGCTA | |
| AACAGCAACGTGCCACTGGAGACCCGGCACTTCCTCTTCAAGACCTCCTCGGGGAGCACACCCTTGTTCAGCAGCTCTTC | |
| CCCGGGATACCCTTTGACCTCAGGAACGGTTTACACGCCCCCGCCCCGCCTGCTGCCCAGGAATACTTTCTCCAGGAAGG | |
| CTTTCAAGCTGAAGAAGCCCTCCAAATACTGCAGCTGGAAATGTGCTGCCCTCTCCGCCATTGCCGCGGCCCTCCTCTTG | |
| GCTATTTTGCTGGCGTATTTCATAGTGCCCTGGTCGTTGAAAAACAGCAGCATAGACAGTGGTGAAGCAGAAGTTGGTCG | |
| GCGGGTAACACAAGAAGTCCCACCAGGGGTGTTTTGGAGGTCACAAATTCACATCAGTCAGCCCCAGTTCTTAAAGTTCA | |
| ACATCTCCCTCGGGAAGGACGCTCTCTTTGGTGTTTACATAAGAAGAGGACTTCCACCATCTCATGCCCAGTATGACTTC | |
| ATGGAACGTCTGGACGGGAAGGAGAAGTGGAGTGTGGTTGAGTCTCCCAGGGAACGCCGGAGCATACAGACCTTGGTTCA | |
| GAATGAAGCCGTGTTTGTGCAGTACCTGGATGTGGGCCTGTGGCATCTGGCCTTCTACAATGATGGAAAAGACAAAGAGA | |
| TGGTTTCCTTCAATACTGTTGTCCTAGATTCAGTGCAGGACTGTCCACGTAACTGCCATGGGAATGGTGAATGTGTGTCC | |
| GGGGTGTGTCACTGTTTCCCAGGATTTCTAGGAGCAGACTGTGCTAAAGCTGCCTGCCCTGTCCTGTGCAGTGGGAATGG | |
| ACAATATTCTAAAGGGACGTGCCAGTGCTACAGCGGCTGGAAAGGTGCAGAGTGCGACGTGCCCATGAATCAGTGCATCG | |
| ATCCTTCCTGCGGGGGCCACGGCTCCTGCATTGATGGGAACTGTGTCTGCTCTGCTGGCTACAAAGGCGAGCACTGTGAG | |
| GAAGTTGATTGCTTGGATCCCACCTGCTCCAGCCACGGAGTCTGTGTGAATGGAGAATGCCTGTGCAGCCCTGGCTGGGG | |
| TGGTCTGAACTGTGAGCTGGCGAGGGTCCAGTGCCCAGACCAGTGCAGTGGGCATGGCACGTACCTGCCTGACACGGGCC | |
| TCTGCAGCTGCGATCCCAACTGGATGGGTCCCGACTGCTCTGTTGAAGTTGTGCTCAGTAGACTGTGGCACTCACGCGTC | |
| TGCATCGGGGGAGCCTGCCGCTGTGAAGAGGGCTGGACAGGCGCAGCGTGTGACCAGCGCGTGTGCCACCCCCGCTGCAT | |
| TGAGCACGGGACCTGTAAAGATGGCAAATGTGAATGCCGAGAGGGCTGGAATGGTGAACACTGCACCATTGGTAGGCAAA | |
| CGGCAGGCACCGAAACAGATGGCTGCCCTGACTTGTGCAACGGTAACGGGAGATGCACACTGGGTCAGAACAGCTGGCAG | |
| TGTGTCTGCCAGACCGGCTGGAGAGGGCCCGGATGCAACGTTGCCATGGAAACTTCCTGTGCTGATAACAAGGATAATGA | |
| GGGAGATGGCCTGGTGGATTGTTTGGACCCTGACTGCTGCCTGCAGTCAGCCTGTCAGAACAGCCTGCTCTGCCGGGGGT | |
| CCCGGGACCCACTGGACATCATTCAGCAGGGCCAGACGGATTGGCCCGCAGTGAAGTCCTTCTATGACCGTATCAAGCTC | |
| TTGGCAGGCAAGGATAGCACCCACATCATTCCTGGAGAGAACCCTTTCAACAGCAGCTTGGTTTCTCTCATCCGAGGCCA | |
| AGTAGTAACTACAGATGGAACTCCCCTGGTCGGTGTGAACGTGTCTTTTGTCAAGTACCCAAAATACGGCTACACCATCA | |
| CCCGCCAGGATGGCACGTTCGACCTGATCGCAAATGGAGGTGCTTCCTTGACTCTACACTTTGAGCGAGCCCCGTTCATG | |
| AGCCAGGAGCGCACTGTGTGGCTGCCGTGGAACAGCTTTTACGCCATGGACACCCTGGTGATGAAGACCGAGGAGAACTC | |
| CATCCCCAGCTGTGACCTCAGTGGCTTTGTCCGGCCTGATCCAATCATCATCTCCTCCCCACTGTCCACCTTCTTTAGTG | |
| CTGCCCCTGGGCAGAATCCCATCGTGCCTGAGACCCAGGTTCTTCATGAAGAAATCGAGCTCCCTGGTTCCAATGTGAAA | |
| CTTCGCTATCTGAGCTCTAGAACTGCAGGGTACAAGTCACTGCTGAAGATCACCATGACCCAGTCCACAGTGCCCCTGAA | |
| CCTCATTAGGGTTCACCTGATGGTGGCTGTCGAGGGGCATCTCTTCCAGAAGTCATTCCAGGCTTCTCCCAACCTGGCCT | |
| CCACCTTCATCTGGGACAAGACAGATGCGTATGGCCAAAGGGTGTATGGACTCTCAGATGCTGTTGTGTCTGTCGGGTTT | |
| GAATATGAGACCTGTCCCAGTCTAATTCTCTGGGAGAAAAGGACAGCCCTCCTTCAGGGATTCGAGCTGGACCCCTCCAA | |
| CCTCGGTGGCTGGTCCCTAGACAAACACCACATCCTCAATGTTAAAAGTGGAATCCTACACAAAGGCACTGGGGAAAACC | |
| AGTTCCTGACCCAGCAGCCTGCCATCATCACCAGCATCATGGGCAATGGTCGCCGCCGGAGCATTTCCTGTCCCAGCTGC | |
| AACGGCCTTGCTGAAGGCAACAAGCTGCTGGCCCCAGTGGCTCTGGCTGTTGGAATCGATGGGAGCCTCTATGTGGGTGA | |
| CTTCAATTACATCCGACGCATCTTTCCCTCTCGAAATGTGACCAGCATCTTGGAGTTACGAAATAAAGAGTTTAAACATA | |
| GCAACAACCCAGCACACAAGTACTACTTGGCAGTGGACCCCGTGTCCGGCTCGCTCTACGTGTCCGACACCAACAGCAGG | |
| AGAATCTACCGCGTCAAGTCTCTGAGTGGAACCAAAGACCTGGCTGGGAATTCGGAAGTTGTGGCAGGGACGGGAGAGCA | |
| GTGTCTACCCTTTGATGAAGCCCGCTGCGGGGATGGAGGGAAGGCCATAGATGCAACCCTGATGAGCCCGAGAGGTATTG | |
| CAGTAGACAAGAATGGGCTCATGTACTTTGTCGATGCCACCATGATCCGGAAGGTTGACCAGAATGGAATCATCTCCACC | |
| CTGCTGGGCTCCAATGACCTCACTGCCGTCCGGCCGCTGAGCTGTGATTCCAGCATGGATGTAGCCCAGGTTCGTCTGGA | |
| GTGGCCAACAGACCTTGCTGTCAATCCCATGGATAACTCCTTGTATGTTCTAGAGAACAATGTCATCCTTCGAATCACCG | |
| AGAACCACCAAGTCAGCATCATTGCGGGACGCCCCATGCACTGCCAAGTTCCTGGCATTGACTACTCACTCAGCAAACTA | |
| GCCATTCACTCTGCCCTGGAGTCAGCCAGTGCCATTGCCATTTCTCACACTGGGGTCCTCTACATCACTGAGACAGATGA | |
| GAAGAAGATTAACCGTCTACGCCAGGTAACAACCAACGGGGAGATCTGCCTTTTAGCTGGGGCAGCCTCGGACTGCGACT | |
| GCAAAAACGATGTCAATTGCAACTGCTATTCAGGAGATGATGCCTACGCGACTGATGCCATCTTGAATTCCCCATCATCC | |
| TTAGCTGTAGCTCCAGATGGTACCATTTACATTGCAGACCTTGGAAATATTCGGATCAGGGCGGTCAGCAAGAACAAGCC | |
| TGTTCTTAATGCCTTCAACCAGTATGAGGCTGCATCCCCCGGAGAGCAGGAGTTATATGTTTTCAACGCTGATGGCATCC | |
| ACCAATACACTGTGAGCCTGGTGACAGGGGAGTACTTGTACAATTTCACATATAGTACTGACAATGATGTCACTGAATTG | |
| ATTGACAATAATGGGAATTCCCTGAAGATCCGTCGGGACAGCAGTGGCATGCCCCGTCACCTGCTCATGCCTGACAACCA | |
| GATCATCACCCTCACCGTGGGCACCAATGGAGGCCTCAAAGTCGTGTCCACACAGAACCTGGAGCTTGGTCTCATGACCT | |
| ATGATGGCAACACTGGGCTCCTGGCCACCAAGAGCGATGAAACAGGATGGACGACTTTCTATGACTATGACCACGAAGGC | |
| CGCCTGACCAACGTGACGCGCCCCACGGGGGTGGTAACCAGTCTGCACCGGGAAATGGAGAAATCTATTACCATTGACAT | |
| TGAGAACTCCAACCGTGATGATGACGTCACTGTCATCACCAACCTCTCTTCAGTAGAGGCCTCCTACACAGTGGTACAAG | |
| ATCAAGTTCGGAACAGCTACCAGCTCTGTAATAATGGTACCCTGAGGGTGATGTATGCTAATGGGATGGGTATCAGCTTC | |
| CACAGCGAGCCCCATGTCCTAGCGGGCACCATCACCCCCACCATTGGACGCTGCAACATCTCCCTGCCTATGGAGAATGG | |
| CTTAAACTCCATTGAGTGGCGCCTAAGAAAGGAACAGATTAAAGGCAAAGTCACCATCTTTGGCAGGAAGCTCCGGGTCC | |
| ATGGAAGAAATCTCTTGTCCATTGACTATGATCGAAATATTCGGACTGAAAAGATCTATGATGACCACCGGAAGTTCACC | |
| CTGAGGATCATTTATGACCAGGTGGGCCGCCCCTTCCTCTGGCTGCCCAGCAGCGGGCTGGCAGCTGTCAACGTGTCATA | |
| CTTCTTCAATGGGCGCCTGGCTGGGCTTCAGCGTGGGGCCATGAGCGAGAGGACAGACATCGACAAGCAAGGCCGCATCG | |
| TGTCCCGCATGTTCGCTGACGGGAAAGTGTGGAGCTACTCCTACCTTGACAAGTCCATGGTCCTCCTGCTTCAGAGCCAA | |
| CGTCAGTATATATTTGAGTATGACTCCTCTGACCGCCTCCTTGCCGTCACCATGCCCAGCGTGGCCCGGCACAGCATGTC | |
| CACACACACCTCCATCGGCTACATCCGTAATATTTACAACCCGCCTGAAAGCAATGCTTCGGTCATCTTTGACTACAGTG | |
| ATGACGGCCGCATCCTGAAGACCTCCTTTTTGGGCACCGGACGCCAGGTGTTCTACAAGTATGGGAAACTCTCCAAGTTA | |
| TCAGAGATTGTCTACGACAGTACCGCCGTCACCTTCGGGTATGACGAGACCACTGGTGTCTTGAAGATGGTCAACCTCCA | |
| AAGTGGGGGCTTCTCCTGCACCATCAGGTACCGGAAGATTGGCCCCCTGGTGGACAAGCAGATCTACAGGTTCTCCGAGG | |
| AAGGCATGGTCAATGCCAGGTTTGACTACACCTATCATGACAACAGCTTCCGCATCGCAAGCATCAAGCCCGTCATAAGT | |
| GAGACTCCCCTCCCCGTTGACCTCTACCGCTATGATGAGATTTCTGGCAAGGTGGAACACTTTGGTAAGTTTGGAGTCAT | |
| CTATTATGACATCAACCAGATCATCACCACTGCCGTGATGACCCTCAGCAAACACTTCGACACCCATGGGCGGATCAAGG | |
| AGGTCCAGTATGAGATGTTCCGGTCCCTCATGTACTGGATGACGGTGCAATATGACAGCATGGGCAGGGTGATCAAGAGG | |
| GAGCTAAAACTGGGGCCCTATGCCAATACCACGAAGTACACCTATGACTACGATGGGGACGGGCAGCTCCAGAGCGTGGC | |
| CGTCAATGACCGCCCGACCTGGCGCTACAGCTATGACCTTAATGGGAATCTCCACTTACTGAACCCAGGCAACAGTGTGC | |
| GCCTCATGCCCTTGCGCTATGACCTCCGGGATCGGATAACCAGACTCGGGGATGTGCAGTACAAAATTGACGACGATGGC | |
| TATCTGTGCCAGAGAGGGTCTGACATCTTCGAATACAATTCCAAGGGCCTCCTAACAAGAGCCTACAACAAGGCCAGCGG | |
| GTGGAGTGTCCAGTACCGCTATGATGGCGTAGGACGGCGGGCTTCCTACAAGACCAACCTGGGCCACCACCTGCAGTACT | |
| TCTACTCTGACCTCCACAACCCGACGCGCATCACCCATGTCTACAATCACTCCAACTCGGAGATTACCTCACTGTACTAC | |
| GACCTCCAGGGCCACCTCTTTGCCATGGAGAGCAGCAGTGGGGAGGAGTACTATGTTGCCTCTGATAACACAGGGACTCC | |
| TCTGGCTGTGTTCAGCATCAACGGCCTCATGATCAAACAGCTGCAGTACACGGCCTATGGGGAGATTTATTATGACTCCA | |
| ACCCCGACTTCCAGATGGTCATTGGCTTCCATGGGGGACTCTATGACCCCCTGACCAAGCTGGTCCACTTCACTCAGCGT | |
| GATTATGATGTGCTGGCAGGACGATGGACCTCCCCAGACTATACCATGTGGAAAAACGTGGGCAAGGAGCCGGCCCCCTT | |
| TAACCTGTATATGTTCAAGAGCAACAATCCTCTCAGCAGTGAGCTAGATTTGAAGAACTACGTGACAGATGTGAAAAGCT | |
| GGCTTGTGATGTTTGGATTTCAGCTTAGCAACATCATTCCTGGCTTCCCGAGAGCCAAAATGTATTTCGTGCCTCCTCCC | |
| TATGAATTGTCAGAGAGTCAAGCAAGTGAGAATGGACAGCTCATTACAGGTGTCCAACAGACAACAGAGAGACATAACCA | |
| GGCCTTCATGGCTCTGGAAGGACAGGTCATTACTAAAAAGCTCCACGCCAGCATCCGAGAGAAAGCAGGTCACTGGTTTG | |
| CCACCACCACGCCCATCATTGGCAAAGGCATCATGTTTGCCATCAAAGAAGGGCGGGTGACCACGGGCGTGTCCAGCATC | |
| GCCAGCGAAGATAGCCGCAAGGTGGCATCTGTGCTGAACAACGCCTACTACCTGGACAAGATGCACTACAGCATCGAGGG | |
| CAAGGACACCCACTACTTTGTGAAGATTGGCTCAGCCGATGGCGACCTGGTCACACTACGCACCACCATCGGCCGCAAGG | |
| TGCTAGAGAGCGGGGTGAACGTGACCGTGTCCCAGCCCACGCTGCTGGTCAACGGCAGGACTCGAAGGTTCACGAACATT | |
| GAGTTCCAGTACTCCACGCTGCTGCTCAGCATCCGCTATGGCCTCACCCCCGACACCCTGGACGAAGAGAAGGCCCGCGT | |
| CCTGGACCAGGCGAGACAGAGGGCCCTGGGCACGGCCTGGGCCAAGGAGCAGCAGAAAGCCAGGGACGGGAGAGAGGGGA | |
| GCCGCCTGTGGACTGAGGGCGAGAAGCAGCAGCTTCTGAGCACCGGGCGCGTGCAAGGGTACGAGGGATATTACGTGCTT | |
| CCCGTGGAGCAATACCCAGAGCTTGCAGACAGTAGCAGCAACATCCAGTTTTTAAGACAGAATGAGATGGGAAAGAGGTA | |
| ACAAAATAATCTGCTGCCATTCCTTGTCTGAATGGCTCAGCAGGAGTAACTGTTATCTCCTCTCCTAAGGAGATGAAGAC | |
| CTAACAGGGGCACTGCGGCTGGGCTGCTTTAGGAGACCAAGTGGCAAGAAAGCTCACATTTTTTGAGTTCAAATGCTACT | |
| GTCCAAGCGAGAAGTCCCTCATCCTGAAGTAGACTAAAGCCCGGCTGAAAATTCCGAGGAAAACAAAACAAACGAATGAA | |
| TGAACAGACACACACAATGTTCCAAGTTCCCCTAAAATATGACCCACTTGTTCTGGGTCTACGCAGAAAAGAGACGCAAA | |
| GTGTCCAAAAGGAACAAAAGAACAAAAACGAATAAGCAAAGAAGAAAACAAACAAAAACAAAACAAAACAAACACACGGA | |
| CCGATAAACAAAGAAGCGAAGATAAGAAAGAAGGCCTCATATCCAATTACCTCACTCATTCACATGTGAGCGACACGCAG | |
| ACATCCGCGAGGGCCAGCGTCACCAGACCAGCTGCGGGACAAACCACTCAGACTGCTTGTAGGACAAATACTTCTGACAT | |
| TTTCGTTTAAGCAAATACAGGTGCATTTAAAACACGACTTTGGGGGTGATTTGTGTGTAGCGCCTGGGGAGGGGGGATAA | |
| AAGAGGAGGAGTGAGCACTGGAAATACTTTTTAAAGAAAAAAAAACATGAGGGAATAAAAGAAATTCCTATCAAAAATCA | |
| AAGTGAAATAATACCATCCAGCACTTAACTCTCAGGTCCCAACTAAGTCTGGCCTGAGCTAATTTATTTGAGCGCAGAGT | |
| GTAAAATTTAATTCAAAATGGTGGCTATAATCACTACAGATAAATTTCATACTCTTTTGTCTTTGGAGATTCCATTGTGG | |
| ACAGTAATACGCAGTTACAGGGTGTAGTCTGTTTAGATTCCGTAGTTCGTGGGTATCAGTTTCGGTAGAGGTGCAGCATC | |
| GTGACACTTTTGCTAACAGGTACCACTTCTGATCACCCTGTACATACATGAGCCGAAAGGCACAATCACTGTTTCAGATT | |
| TAAAATTATTAGTGTGTTTGTTTGGTCCAGAAACTGAGACAATCACATGACAGTCACCACGAGGAGAGAAAATTTAAAAA | |
| ATAAAAATAAAAACAAAAAAAATTTTAAAAATTAAAAAAACAAAAATAAAGTCTAATAAGAACTTTGGTACAGGAACTTT | |
| TTTGTAATATACATGTATGAATTGTTCATCGAGTTTTTATATTAATTTTAATTTGCTGCTAAGCAAAGACTAGGGACAGG | |
| CAAAGATAATTTATGGCAAAGTGTTTAAATTGTTTATACATAAATAAAGTCTCTAAAACTCCTGTG | |

The FCTR3b polypeptide (SEQ ID NO:8) encoded by SEQ ID NO:7 is 2733 amino acid residues and is presented using the one-letter code in Table 3D. The protein has a predicted molecular weight of 303424.3 daltons.

TABLE 3D
Encoded FCTR3b protein sequence.

| | |
|---|---|
| MDVKDRRHRSLTRGRCGKECRYTSSSLDSEDCRVPTQKSYSSSETLKAYDHDSRMHYGNRVTDLIHRESDEFPRQGTNFTLAE | (SEQ ID NO:8) |
| LGICEPSPHRSGYCSDMGILHQGYSLSTGSDADSDTEGGMSPEHAIRLWGRGIKSRRSSGLSSRENSALTLTDSDNENKSDDE | |
| NGRPIPPTSSPSLLPSAQLPSSHNPPPVSCQMPLLDSNTSHQIMDTNPDEEFSPNSYLLRACSGPQQASSSGPPNHHSQSTLR | |
| PPLPPPHNHTLSHHHSSANSLNRNSLTNRRSQIHAPAPAPNDLATTPESVQLQDSWVLNSNVPLETRHFLFKTSSGSTPLFSS | |
| SSPGYPLTSGTVYTPPPRLLPRNTFSRKAFKLKKPSKYCSWKCAALSAIAAALLLAILLAYFIVPWSLKNSSIDSGEAEVGRR | |
| VTQEVPPGVFWRSQIHISQPQFLKFNISLGKDALFGVYIRRGLPPSHAQYDFMERLDGKEKWSVVESPRERRSIQTLVQNEAV | |
| FVQYLDVGLWHLAFYNDGKDKEMVSFNTVVLDSVQDCPRNCHGNGECVSGVCHCFPGFLGADCAKAACPVLCSGNGQYSKGTC | |
| QCYSGWKGAECDVPMNQCIDPSCGGHGSCIDGNCVCSAGYKGEHCEEVDCLDPTCSSHGVCVNGECLCSPGWGGLNCELARVQ | |
| CPDQCSGHGTYLPDTGLCSCDPNWMGPDCSVEVCSVDCGTHGVCIGGACRCEEGWTGAACDQRVCHPRCIEHGTCKDGKCECR | |
| EGWNGEHCTIGRQTAGTETDGCPDLCNGNGRCTLGQNSWQCVCQTGWRGPGCNVAMETSCADNKDNEGDGLVDCLDPDCCLQS | |
| ACGNSLLCRGSRDPLDIIQQGQTDWPAVKSFYDRIKLLAGKDSTHIIPGENPFNSSLVSLIRGQVVTTDGTPLVGVNVSFVKY | |
| PKYGYTITRQDGTFDLIANGGASLTLHFERAPFMSQERTVWLPWNSFYAMDTLVMKTEENSIPSCDLSGFVRPDPIIISSPLS | |
| TFFSAAPGQNPIVPETQVLHEEIELPGSNVKLRYLSSRTAGYKSLLKITMTQSTVPLNLIRVHLMVAVEGHLFQKSFQASPNL | |
| ASTFIWDKTDAYGQRVYGLSDAVVSVGFEYETCPSLILWEKRTALLQGFELDPSNLGGWSLDKHHILNVKSGILHKGTGENQF | |
| LTQQPAIITSIMGNGRRRSISCPSCNGLAEGNKLLAPVALAVGIDGSLYVGDFNYIRRIFPSRNVTSILELRNKEFKHSNNPA | |
| HKYYLAVDPVSGSLYVSDTNSRRIYRVKSLSGTKDLAGNSEVVAGTGEQCLPFDEARCGDGGKAIDATLMSPRGIAVDKNGLM | |
| YFVDATMIRKVDQNGIISTLLGSNDLTAVRPLSCDSSMDVAQVRLEWPTDLAVNPMDNSLYVLENNVILRITENHQVSIIAGR | |
| PMHCQVPGIDYSLSKLAIHSALESASAIAISHTGVLYITETDEKKINRLRQVTTNGEICLLAGAASDCDCKNDVNCNCYSGDD | |
| AYATDAILNSPSSLAVAPDGTIYIADLGNIRIRAVSKNKPVLNAFNQYEAASPGEQELYVFNADGIHQYTVSLVTGEYLYNFT | |
| YSTDNDVTELIDNNGNSLKIRRDSSGMPRHLLMPDNQIITLTVGTNGGLKVVSTQNLELGLMTYDGNTGLLATKSDETGWTTF | |
| YDYDHEGRLTNVTRPTGVVTSLHREMEKSITIDIENSNRDDDVTVITNLSSVEASYTVVQDQVRNSYQLCNNGTLRVMYANGM | |
| GISFHSEPHVLAGTATPTIGRCNISLPMENGLNSIEWRLRKEQIKGKVTIFGRKLRVHGRNLLSIDYDRNIRTEKIYDDHRKF | |
| TLRIIYDQVGRPFLWLPSSGLAAVNVSYFFNGRLAGLQRGAMSERTDIDKQGRIVSRMFADGKVWSYSYLDKSMVLLLQSQRQ | |
| YIFEYDSSDRLLAVTMPSVARHSMSTHTSIGYIRNIYNPPESNASVIFDYSDDGRILKTSFLGTGRQVFYKYGKLSKLSEIVY | |
| DSTAVTFGYDETTGVLKMVNLQSGGFSCTIRYRKIGPLVDKQIYRFSEEGMVNARFDYTYHDNSFRIASIKPVISETPLPVDL | |
| YRYDEISGKVEHFGKFGVIYYDINQIITTAVMTLSKHFDTHGRIKEVQYEMFRSLMYWMTVQYDSMGRVIKRELKLGPYANTT | |
| KYTYDYDGDGQLQSVAVNDRPTWRYSYDLNGNLHLLNPGNSVRLMPLRYDLRDRITRLGDVQYKIDDDGYLCQRGSDIFEYNS | |
| KGLLTRAYNKASGWSVQYRYDGVGRRASYKTNLGHHLQYFYSDLHNPTRITHVYNHSNSEITSLYYDLQGHLFAMESSSGEEY | |
| YVASDNTGTPLAVFSINGLMIKQLQYTAYGEIYYDSNPDFQMVIGFHGGLYDPLTKLVHFTQRDYDVLAGRWTSPDYTMWKNV | |
| GKEPAPFNLYMFKSNNPLSSELDLKNYVTDVKSWLVMFGFQLSNIIPGFPRAKMYFVPPPYELSESQASENGQLITGVQQTTE | |
| RHNQAFMALEGQVITKKLHASIREKAGHWFATTTPIIGKGIMFAIKEGRVTTGVSSIASEDSRKVASVLNNAYYLDKMHYSIE | |
| GKDTHYFVKIGSADGDLVTLGTTIGRKVLESGVNVTVSQPTLLVNGRTRRFTNIEFQYSTLLLSIRYGLTPDTLDEEKARVLD | |
| QARQRALGTAWAKEQQKARDGREGSRLWTEGEKQQLLSTGRVQGYEGYYVLPVEQYPELADSSSNIQFLRQNEMGKR | |

In further alternative embodiments the italicized bases in the 5′ end of the FCTR3b sequence in table 3C is a variable region. This region can be substituted for in other embodiments of FCTR3. The nucleotide sequence for 9823 bp FCTR3c (also referred to herein as 10129612.0.154) has the same nucleotide sequence as FCTR3b except that the italicized region is replaced with the 201 base sequence shown in Table 3E. An ORF for the total FCTR3c nucleotide sequence was identified beginning with an ATG initiation codon at nucleotides 277-280 and ending with a TAG codon at nucleotides 8473-8475. This is the same open reading frame that is shown in Table 3C, with the corresponding base numbers for FCTR3c. This open reading frame will translate the same amino acid sequence as shown in Table 3C for FCTR3b.

TABLE 3E
Encoded FCTR3c 5′end nucleotide sequence.

| | |
|---|---|
| GCTCCAAAGCGAGCTGGGACCGAAGACTCTAGGCTAAGTTATCTATGTAGATGGTGTCAGGGAGCGAAGCTACTGACCGA | (SEQ ID NO:9) |
| GCTGCTGTTACATCCAGCTTTTTAATTGCCTAAGCGGTCTGGGGCTTGCTTCGTCATTTGGCTTTGCTGTGGAGCACTCC | |
| TGTAAAGCCAGCTGAATTGTACATCGAAGATCCACCCTTTT | |

In yet another embodiment, the italicized region shown in the 5′ end of the sequence in Table 3C can be replaced with the sequence shown in Table 3F to form 9823 bp FCTR3d (also referred to herein as 10129612.0.67). An ORF was identified beginning with an ATG initiation codon at nucleotides 277-280 and ending with a TAG codon at nucleotides 8473-8475. This is the same open reading frame that is shown in Table 3C, with the corresponding base numbers for FCTR3d. This open reading frame will translate the same amino acid sequence as shown in Table 3D for FCTR3b.

TABLE 3F
Encoded FCTR3d 5′ end nucleotide sequence.

| | |
|---|---|
| GCTCCAAAGCGAGCTGGGACCGAAGACTCTAGGCTA | (SEQ ID NO:10) |
| AGTTATCTATGTAGATGGTGTCAGGGAGCGAAGCTA | |
| CTGACCGAGCTGCTGTTACATCCAGCTTTTTAATTG | |
| CCTAAGCGGTCTGGGGCTTGCTTCGTCATTTGGCTT | |
| TGCTGTGGAGCACTCCTGTAAAGCCAGCTGAATTGT | |
| ACATCGAAGATCCACCCTTTT | |

In yet another embodiment, the italicized region shown in the 5′ end of the sequence in Table 3C can be replaced with the sequence shown in Table 3G to form 9765 bp FCTR3e (also referred to as 10129612.0.258). An ORF was identified beginning with an ATG initiation codon at nucleotides 210-212 and ending with a TAG codon at nucleotides 8408-8410. This is the same open reading frame that is shown in Table 3C, with the corresponding base numbers for FCTR3e. This open reading frame will translate the same amino acid sequence as shown in Table 3D for FCTR3b.

TABLE 3G
Encoded FCTR3e 5′ end nucleotide sequence.

| | |
|---|---|
| CCAGCATTAGATGAGTTGACAAAAATGCAGTTTCAG | (SEQ ID NO:11) |
| CTCTGAAGGTCTGAAAGATTCTGCTGCAACTAAAGC | |
| TCTGAAGATTCTGCTACAACTATGACATCCATTTTC | |
| TCCCACTTCAGACAGGATGAATACAA | |

In yet another embodiment another FCTR3a homolog, FCTR3f (also referred to as 10129612.0.352) was found having the 9729bp sequence shown in Table 3H. An ORF was identified beginning with an ATG initiation codon at nucleotides 210-212 and ending with a TAG codon at nucleotides 8382-8384. A putative untranslated region upstream from the initiation codon and downstream from the termination codon is underlined in Table 3G, and the start and stop codons are in bold letters.

TABLE 3H
Encoded FCTR3f nucleotide sequence.

| | |
|---|---|
| CCAGCATTAGATGAGTTGACAAAAATGCAGTTTCAGCTCTGAAGGTCTGAAAGATTCTGCTGCAACTAAAGCTCTGAAGA | (SEQ ID NO:12) |
| TTCTGCTACAACTATGACATCCATTTTCTCCCACTTCAGACAGGATGAATACAAGGTGGCAAAGTGACAAGTGCCAAAAC | |
| TCAGGCCTGACTTTCCTGAAAACATCAGCATTCTGCCATATCTGGAATAATGGATGTAAAGGACCGGCGACACCGCTCTT | |
| TGACCAGAGGACGCTGTGGCAAAGAGTGTCGCTACACAAGCTCCTCTCTGGACAGTGAGGACTGCCGGGTGCCCACACAG | |
| AAATCCTACAGCTCCAGTGAGACTCTGAAGGCCTATGACCATGACAGCAGGATGCACTATGGAAACCGAGTCACAGACCT | |
| CATCCACCGGGAGTCAGATGAGTTTCCTAGACAAGGAACCAACTTCACCCTTGCCGAACTGGGCATCTGTGAGCCCTCCC | |
| CACACCGAAGCGGCTACTGCTCCGACATGGGGATCCTTCACCAGGGCTACTCCCTTAGCACAGGGTCTGACGCCGACTCC | |
| GACACCGAGGGAGGGATGTCTCCAGAACACGCCATCAGACTGTGGGGCAGAGGGATAAAATCCAGGCGCAGTTCCGGCCT | |
| GTCCAGTCGTGAAAACTCGGCCCTTACCCTGACTGACTCTGACAACGAAAACAAATCAGATGATGAGAACGGTCGTCCCA | |
| TTCCACCTACATCCTCGCCTAGTCTCCTCCCATCTGCTCAGCTGCCTAGCTCCCATAATCCTCCACCAGTTAGCTGCCAG | |
| ATGCCATTGCTAGACAGCAACACCTCCCATCAAATCATGGACACCAACCCTGATGAGGAATTCTCCCCCAATTCATACCT | |
| GCTCAGAGCATGCTCAGGGCCCCAGCAAGCCTCCAGCAGTGGCCCTCCGAACCACCACAGCCAGTCGACTCTGAGGCCCC | |
| CTCTCCCACCCCCTCACAACCACACGCTGTCCCATCACCACTCGTCCGCCAACTCCCTCAACAGGAACTCACTGACCAAT | |
| CGGCGGAGTCAGATCCACGCCCCGGCCCCAGCGCCCAATGACCTGGCCACCACACCAGAGTCCGTTCAGCTTCAGGACAG | |
| CTGGGTGCTAAACAGCAACGTGCCACTGGAGACCCGGCACTTCCTCTTCAAGACCTCCTCGGGGAGCACACCCTTGTTCA | |
| GCAGCTCTTCCCCGGGATACCCTTTGACCTCAGGAACGGTTTACACGCCCCCGCCCCGCCTGCTGCCCAGGAATACTTTC | |
| TCCAGGAAGGCTTTCAAGCTGAAGAAGCCCTCCAAATACTGCAGCTGGAAATGTGCTGCCCTCTCCGCCATTGCCGCGGC | |
| CCTCCTCTTGGCTATTTTGCTGGCGTATTTCATAGTGCCCTGGTCGTTGAAAAACAGCAGCATAGACAGTGGTGAAGCAG | |
| AAGTTGGTCGGCGGGTAACACAAGAAGTCCCACCAGGGGTGTTTTGGAGGTCACAAATTCACATCAGTCAGCCCCAGTTC | |
| TTAAAGTTCAACATCTCCCTCGGGAAGGACGCTCTCTTTGGTGTTTACATAAGAAGAGGACTTCCACCATCTCATGCCCA | |
| GTATGACTTCATGGAACGTCTGGACGGGAAGGAGAAGTGGAGTGTGGTTGAGTCTCCCAGGGAACGCCGGAGCATACAGA | |
| CCTTGGTTCAGAATGAAGCCGTGTTTGTGCAGTACCTGGATGTGGGCCTGTGGCATCTGGCCTTCTACAATGATGGAAAA | |
| GACAAAGAGATGGTTTCCTTCAATACTGTTGTCCTAGATTCAGTGCAGGACTGTCCACGTAACTGCCATGGGAATGGTGA | |
| ATGTGTGTCCGGGGTGTGTCACTGTTTCCCAGGATTTCTAGGAGCAGACTGTGCTAAAGCTGCCTGCCCTGTCCTGTGCA | |
| GTGGGAATGGACAATATTCTAAAGGGACGTGCCAGTGCTACAGCGGCTGGAAAGGTGCAGAGTGCGACGTGCCCATGAAT | |
| CAGTGCATCGATCCTTCCTGCGGGGGCCACGGCTCCTGCATTGATGGGAACTGTGTCTGCTCTGCTGGCTACAAAGGCGA | |
| GCACTGTGAGGAAGTTGATTGCTTGGATCCCACCTGCTCCAGCCACGGAGTCTGTGTGAATGGAGAATGCCTGTGCAGCC | |
| CTGGCTGGGGTGGTCTGAACTGTGAGCTGGCGAGGGTCCAGTGCCCAGACCAGTGCAGTGGGCATGGCACGTACCTGCCT | |
| GACACGGGCCTCTGCAGCTGCGATCCCAACTGGATGGGTCCCGACTGCTCTGTTGAAGTGTGCTCAGTAGACTGTGGCAC | |
| TCACGGCGTCTGCATCGGGGGAGCCTGCCGCTGTGAAGAGGGCTGGACAGGCGCAGCGTGTGACCAGCGCGTGTGCCACC | |
| CCCGCTGCATTGAGCATGGGACCTGTAAAGATGGCAAATGTGAATGCCGAGAGGGCTGGAATGGTGAACACTGCACCATT | |
| GATGGCTGCCCTGACTTGTGCAACGGTAACGGGAGATGCACACTGGGTCAGAACAGCTGGCAGTGTGTCTGCCAGACCGG | |
| CTGGAGAGGGCCCGGATGCAACGTTGCCATGGAAACTTCCTGTGCTGATAACAAGGATAATGAGGGAGATGGCCTGGTGG | |
| ATTGTTTGGACCCTGACTGCTGCCTGCAGTCAGCCTGTCAGAACAGCCTGCTCTGCCGGGGGTCCCGGGACCCACTGGAC | |
| ATCATTCAGCAGGGCCAGACGGATTGGCCCGCAGTGAAGTCCTTCTATGACCGTATCAAGCTCTTGGCAGGCAAGGATAG | |
| CACCCACATCATTCCTGGAGAGAACCCTTTCAACAGCAGCTTGGTTTCTCTCATCCGAGGCCAAGTAGTAACTACAGATG | |
| GAACTCCCCTGGTCGGTGTGAACGTGTCTTTTGTCAAGTACCCAAAATACGGCTACACCATCACCCGCCAGGATGGCACG | |
| TTCGACCTGATCGCAAATGGAGGTGCTTCCTTGACTCTACACTTTGAGCGAGCCCCGTTCATGAGCCAGGAGCGCACTGT | |
| GTGGCTGCCGTGGAACAGCTTTTACGCCATGGACACCCTGGTGATGAAGACCGAGGAGAACTCCATCCCCAGCTGTGACC | |
| TCAGTGGCTTTGTCCGGCCTGATCCAATCATCATCTCCTCCCCACTGTCCACCTTCTTTAGTGCTGCCCCTGGGCAGAAT | |
| CCCATCGTGCCTGAGACCCAGGTTCTTCATGAAGAAATCGAGCTCCCTGGTTCCAATGTGAAACTTCGCTATCTGAGCTC | |
| TAGAACTGCAGGGTACAAGTCACTGCTGAAGATCACCATGACCCAGTCCACAGTGCCCCTGAACCTCATTAGGGTTCACC | |
| TGATGGTGGCTGTCGAGGGGCATCTCTTCCAGAAGTCATTCCAGGCTTCTCCCAACCTGGCCTCCACCTTCATCTGGGAC | |
| AAGACAGATGCGTATGGCCAAAGGGTGTATGGACTCTCAGATGCTGTTGTGTCTGTCGGGTTTGAATATGAGACCTGTCC | |
| CAGTCTAATTCTCTGGGAGAAAAGGACAGCCCTCCTTCAGGGATTCGAGCTGGACCCCTCCAACCTCGGTGGCTGGTCCC | |
| TAGACAAACACCACATCCTCAATGTTAAAAGTGGAATCCTACACAAAGGCACTGGGGAAAACCAGTTCCTGACCCAGCAG | |
| CCTGCCATCATCACCAGCATCATGGGCAATGGTCGCCGCCGGAGCATTTCCTGTCCCAGCTGCAACGGCCTTGCTGAAGG | |
| CAACAAGCTGCTGGCCCCAGTGGCTCTGGCTGTTGGAATCGATGGGAGCCTCTATGTGGGTGACTTCAATTACATCCGAC | |
| GCATCTTTCCCTCTCGAAATGTGACCAGCATCTTGGAGTTACGAAATAAAGAGTTTAAACATAGCAACAACCCAGCACAC | |
| AAGTACTACTTGGCAGTGGACCCCGTGTCCGGCTCGCTCTACGTGTCCGACACCAACAGCAGGAGAATCTACCGCGTCAA | |
| GTCTCTGAGTGGAACCAAAGACCTGGCTGGGAATTCGGAAGTTGTGGCAGGGACGGGAGAGCAGTGTCTACCCTTTGATG | |
| AAGCCCGCTGCGGGGATGGAGGGAAGGCCATAGATGCAACCCTGATGAGCCCGAGAGGTATTGCAGTAGACAAGAATGGG | |
| CTCATGTACTTTGTCGATGCCACCATGATCCGGAAGGTTGACCAGAATGGAATCATCTCCACCCTGCTGGGCTCCAATGA | |
| CCTCACTGCCGTCCGGCCGCTGAGCTGTGATTCCAGCATGGATGTAGCCCAGGTTCGTCTGGAGTGGCCAACAGACCTTG | |
| CTGTCAATCCCATGGATAACTCCTTGTATGTTCTAGAGAACAATGTCATCCTTCGAATCACCGAGAACCACCAAGTCAGC | |
| ATCATTGCGGGACGCCCCATGCACTGCCAAGTTCCTGGCATTGACTACTCACTCAGCAAACTAGCCATTCACTCTGCCCT | |
| GGAGTCAGCCAGTGCCATTGCCATTTCTCACACTGGGGTCCTCTACATCACTGAGACAGATGAGAAGAAGATTAACCGTC | |
| TACGCCAGGTAACAACCAACGGGGAGATCTGCCTTTTAGCTGGGGCAGCCTCGGACTGCGACTGCAAAAACGATGTCAAT | |
| TGCAACTGCTATTCAGGAGATGATGCCTACGCGACTGATGCCATCTTGAATTCCCCATCATCCTTAGCTGTAGCTCCAGA | |
| TGGTACCATTTACATTGCAGACCTTGGAAATATTCGGATCAGGGCGGTCAGCAAGAACAAGCCTGTTCTTAATGCCTTCA | |
| ACCAGTATGAGGCTGCATCCCCCGGAGAGCAGGAGTTATATGTTTTCAACGCTGATGGCATCCACCAATACACTGTGAGC | |
| CTGGTGACAGGGGAGTACTTGTACAATTTCACATATAGTACTGACAATGATGTCACTGAATTGATTGACAATAATGGGAA | |
| TTCCCTGAAGATCCGTCGGGACAGCAGTGGCATGCCCCGTCACCTGCTCATGCCTGACAACCAGATCATCACCCTCACCG | |
| TGGGCACCAATGGAGGCCTCAAAGTCGTGTCCACACAGAACCTGGAGCTTGGTCTCATGACCTATGATGGCAACACTGGG | |
| CTCCTGGCCACCAAGAGCGATGAAACAGGATGGACGACTTTCTATGACTATGACCACGAAGGCCGCCTGACCAACGTGAC | |
| GCGCCCCACGGGGGTGGTAACCAGTCTGCACCGGGAAATGGAGAAATCTATTACCATTGACATTGAGAACTCCAACCGTG | |
| ATGATGACGTCACTGTCATCACCAACCTCTCTTCAGTAGAGGCCTCCTACACAGTGGTACAAGATCAAGTTCGGAACAGC | |
| TACCAGCTCTGTAATAATGGTACCCTGAGGGTGATGTATGCTAATGGGATGGGTATCAGCTTCCACAGCGAGCCCCATGT | |
| CCTAGCGGGCACCATCACCCCCACCATTGGACGCTGCAACATCTCCCTGCCTATGGAGAATGGCTTAAACTCCATTGAGT | |
| GGCGCCTAAGAAAGGAACAGATTAAAGGCAAAGTCACCATCTTTGGCAGGAAGCTCCGGGTCCATGGAAGAAATCTCTTG | |
| TCCATTGACTATGATCGAAATATTCGGACTGAAAAGATCTATGATGACCACCGGAAGTTCACCCTGAGGATCATTTATGA | |
| CCAGGTGGGCCGCCCCTTCCTCTGGCTGCCCAGCAGCGGGCTGGCAGCTGTCAACGTGTCATACTTCTTCAATGGGCGCC | |
| TGGCTGGGCTTCAGCGTGGGGCCATGAGCGAGAGGACAGACATCGACAAGCAAGGCCGCATCGTGTCCCGCATGTTCGCT | |
| GACGGGAAAGTGTGGAGCTACTCCTACCTTGACAAGTCCATGGTCCTCCTGCTTCAGAGCCAACGTCAGTATATATTTGA | |
| GTATGACTCCTCTGACCGCCTCCTTGCCGTCACCATGCCCAGCGTGGCCCGGCACAGCATGTCCACACACACCTCCATCG | |
| GCTACATCCGTAATATTTACAACCCGCCTGAAAGCAATGCTTCGGTCATCTTTGACTACAGTGATGACGGCCGCATCCTG | |
| AAGACCTCCTTTTTGGGCACCGGACGCCAGGTGTTCTACAAGTATGGGAAACTCTCCAAGTTATCAGAGATTGTCTACGA | |
| CAGTACCGCCGTCACCTTCGGGTATGACGAGACCACTGGTGTCTTGAAGATGGTCAACCTCCAAAGTGGGGGCTTCTCCT | |
| GCACCATCAGGTACCGGAAGATTGGCCCCCTGGTGGACAAGCAGATCTACAGGTTCTCCGAGGAAGGCATGGTCAATGCC | |
| AGGTTTGACTACACCTATCATGACAACAGCTTCCGCATCGCAAGCATCAAGCCCGTCATAAGTGAGACTCCCCTCCCCGT | |
| TGACCTCTACCGCTATGATGAGATTTCTGGCAAGGTGGAACACTTTGGTAAGTTTGGAGTCATCTATTATGACATCAACC | |
| AGATCATCACCACTGCCGTGATGACCCTCAGCAAACACTTCGACACCCATGGGCGGATCAAGGAGGTCCAGTATGAGATG | |
| TTCCGGTCCCTCATGTACTGGATGACGGTGCAATATGACAGCATGGGCAGGGTGATCAAGAGGGAGCTAAAACTGGGGCC | |
| CTATGCCAATACCACGAAGTACACCTATGACTACGATGGGGACGGGCAGCTCCAGAGCGTGGCCGTCAATGACCGCCCGA | |
| CCTGGCGCTACAGCTATGACCTTAATGGGAATCTCCACTTACTGAACCCAGGCAACAGTGTGCGCCTCATGCCCTTGCGC | |
| TATGACCTCCGGGATCGGATAACCAGACTCGGGGATGTGCAGTACAAAATTGACGACGATGGCTATCTGTGCCAGAGAGG | |
| GTCTGACATCTTCGAATACAATTCCAAGGGCCTCCTAACAAGAGCCTACAACAAGGCCAGCGGGTGGAGTGTCCAGTACC | |
| GCTATGATGGCGTAGGACGGCGGGCTTCCTACAAGACCAACCTGGGCCACCACCTGCAGTACTTCTACTCTGACCTCCAC | |
| AACCCGACGCGCATCACCCATGTCTACAATCACTCCAACTCGGAGATTACCTCACTGTACTACGACCTCCAGGGCCACCT | |
| CTTTGCCATGGAGAGCAGCAGTGGGGAGGAGTACTATGTTGCCTCTGATAACACAGGGACTCCTCTGGCTGTGTTCAGCA | |
| TCAACGGCCTCATGATCAAACAGCTGCAGTACACGGCCTATGGGGAGATTTATTATGACTCCAACCCCGACTTCCAGATG | |
| GTCATTGGCTTCCATGGGGGACTCTATGACCCCCTGACCAAGCTGGTCCACTTCACTCAGCGTGATTATGATGTGCTGGC | |
| AGGACGATGGACCTCCCCAGACTATACCATGTGGAAAAACGTGGGCAAGGAGCCGGCCCCCTTTAACCTGTATATGTTCA | |
| AGAGCAACAATCCTCTCAGCAGTGAGCTAGATTTGAAGAACTACGTGACAGATGTGAAAAGCTGGCTTGTGATGTTTGGA | |
| TTTCAGCTTAGCAACATCATTCCTGGCTTCCCGAGAGCCAAAATGTATTTCGTGCCTCCTCCCTATGAATTGTCAGAGAG | |
| TCAAGCAAGTGAGAATGGACAGCTCATTACAGGTGTCCAACAGACAACAGAGAGACATAACCAGGCCTTCATGGCTCTGG | |
| AAGGACAGGTCATTACTAAAAAGCTCCACGCCAGCATCCGAGAGAAAGCAGGTCACTGGTTTGCCACCACCACGCCCATC | |
| ATTGGCAAAGGCATCATGTTTGCCATCAAAGAAGGGCGGGTGACCACGGGCGTGTCCAGCATCGCCAGCGAAGATAGCCG | |
| CAAGGTGGCATCTGTGCTGAACAACGCCTACTACCTGGACAAGATGCACTACAGCATCGAGGGCAAGGACACCCACTACT | |
| TTGTGAAGATTGGCTCAGCCGATGGCGACCTGGTCACACTAGGCACCACCATCGGCCGCAAGGTGCTAGAGAGCGGGGTG | |
| AACGTGACCGTGTCCCAGCCCACGCTGCTGGTCAACGGCAGGACTCGAAGGTTCACGAACATTGAGTTCCAGTACTCCAC | |
| GCTGCTGCTCAGCATCCGCTATGGCCTCACCCCCGACACCCTGGACGAAGAGAAGGCCCGCGTCCTGGACCAGGCGAGAC | |
| AGAGGGCCCTGGGCACGGCCTGGGCCAAGGAGCAGCAGAAAGCCAGGGACGGGAGAGAGGGGAGCCGCCTGTGGACTGAG | |
| GGCGAGAAGCAGCAGCTTCTGAGCACCGGGCGCGTGCAAGGGTACGAGGGATATTACGTGCTTCCCGTGGAGCAATACCC | |
| AGAGCTTGCAGACAGTAGCAGCAACATCCAGTTTTTAAGACAGAATGAGATGGGAAAGAGGTAACAAAATAATCTGCTGC | |
| CATTCCTTGTCTGAATGGCTCAGCAGGAGTAACTGTTATCTCCTCTCCTAAGGAGATGAAGACCTAACAGGGGCACTGCG | |
| GCTGGGCTGCTTTAGGAGACCAAGTGGCAAGAAAGCTCACATTTTTTGAGTTCAAATGCTACTGTCCAAGCGAGAAGTCC | |
| CTCATCCTGAAGTAGACTAAAGCCCGGCTGAAAATTCCGAGGAAAACAAAACAAACGAATGAATGAACAGACACACACAA | |
| TGTTCCAAGTTCCCCTAAAATATGACCCACTTGTTCTGGGTCTACGCAGAAAAGAGACGCAAAGTGTCCAAAAGGAACAA | |
| AAGAACAAAAACGAATAAGCAAAGAAGAAAACAAACAAAAACAAAACAAAACAAACACACGGACCGATAAACAAAGAAGC | |
| GAAGATAAGAAAGAAGGCCTCATATCCAATTACCTCACTCATTCACATGTGAGCGACACGCAGACATCCGCGAGGGCCAG | |
| CGTCACCAGACCAGCTGCGGGACAAACCACTCAGACTGCTTGTAGGACAAATACTTCTGACATTTTCGTTTAAGCAAATA | |
| CAGGTGCATTTAAAACACGACTTTGGGGGTGATTTGTGTGTAGCGCCTGGGGAGGGGGGATAAAAGAGGAGGAGTGAGCA | |
| CTGGAAATACTTTTTAAAGAAAAAAAAACATGAGGGAATAAAAGAAATTCCTATCAAAAATCAAAGTGAAATAATACCAT | |
| CCAGCACTTAACTCTCAGGTCCCAACTAAGTCTGGCCTGAGCTAATTTATTTGAGCGCAGAGTGTAAAATTTAATTCAAA | |
| ATGGTGGCTATAATCACTACAGATAAATTTCATACTCTTTTGTCTTTGGAGATTCCATTGTGGACAGTAATACGCAGTTA | |
| CAGGGTGTAGTCTGTTTAGATTCCGTAGTTCGTGGGTATCAGTTTCGGTAGAGGTGCAGCATCGTGACACTTTTGCTAAC | |
| AGGTACCACTTCTGATCACCCTGTACATACATGAGCCGAAAGGCACAATCACTGTTTCAGATTTAAAATTATTAGTGTGT | |
| TTGTTTGGTCCAGAAACTGAGACAATCACATGACAGTCACCACGAGGAGAGAAAATTTAAAAAATAAAAATAAAAACAAA | |
| AAAAATTTTAAAAATTAAAAAAACAAAAATAAAGTCTAATAAGAACTTTGGTACAGGAACTTTTTTGTAATATACATGTA | |
| TGAATTGTTCATCGAGTTTTTATATTAATTTTAATTTGCTGCTAAGCAAAGACTAGGGACAGGCAAAGATAATTTATGGC | |
| AAAGTGTTTAAATTGTTTATACATAAATAAAGTCTCTAAAACTCCTGTG | |

The FCTR3f polypeptide (SEQ ID NO: 13) encoded by SEQ ID NO:12 is 2724 amino acid residues long and is presented using the one-letter code in Table 3I. This sequence differs from FCTR3b in that it is missing amino acids 758-766 from that polypeptide.

TABLE 3I
Encoded FCTR3f protein sequence.

| | |
|---|---|
| MDVKDRRHRSLTRGRCGKECRYTSSSLDSEDCRVPT | (SEQ ID NO:13) |
| QKSYSSSETLKAYDHDSRMHYGNRVTDLIHRESDEF | |
| PRQGTNFTLAELGICEPSPHRSGYCSDMGILHQGYS | |
| LSTGSDADSDTEGGMSPEHAIRLWGRGIKSRRSSGL | |
| SSRENSALTLTDSDNENKSDDENGRPIPPTSSPSLL | |
| PSAQLPSSHNPPPVSCQMPLLDSNTSHQIMDTNPDE | |
| EFSPNSYLLRACSGPQQASSSGPPNHHSQSTLRPPL | |
| PPPHNHTLSHHHSSANSLNRNSLTNRRSQIHAPAPA | |
| PNDLATTPESVQLQDSWVLNSNVPLETRHFLFKTSS | |
| GSTPLFSSSSPGYPLTSGTVYTPPPRLLPRNTFSRK | |
| AFKLKKPSKYCSWKCAALSAIAAALLLAILLAYFIV | |
| PWSLKNSSIDSGEAEVGRRVTQEVPPGVFWRSQIHI | |
| SQPQFLKFNISLGKDALFGVYIRRGLPPSHAQYDFM | |
| ERLDGKEKWSVVESPRERRSIQTLVQNEAVFVQYLD | |
| VGLWHLAFYNDGKDKEMVSFNTVVLDSVQDCPRNCH | |
| GNGECVSGVCHCFPGFLGADCAKAACPVLCSGNGQY | |
| SKGTCQCYSGWKGAECDVPMNQCIDPSCGGHGSCID | |
| GNCVCSAGYKGEHCEEVDCLDPTCSSHGVCVNGECL | |
| CSPGWGGLNCELARVQCPDQCSGHGTYLPDTGLCSC | |
| DPNWMGPDCSVEVCSVDCGTHGVCIGGACRCEEGWT | |
| GAACDQRVCHPRCIEHGTCKDGKCECREGWNGEHCT | |
| IDGCPDLCNGNGRCTLGQNSWQCVCQTGWRGPGCNV | |
| AMETSCADNKDNEGDGLVDCLDPDCCLQSACQNSLL | |
| CRGSRDPLDIIQQGQTDWPAVKSFYDRIKLLAGKDS | |
| THIIPGENPFNSSLVSLIRGQVVTTDGTPLVGVNVS | |
| FVKYPKYGYTITRQDGTFDLIANGGASLTLHFERAP | |
| FMSQERTVWLPWNSFYAMDTLVMKTEENSIPSCDLS | |
| GFVRPDPIIISSPLSTFFSAAPGQNPIVPETQVLHE | |
| EIELPGSNVKLRYLSSRTAGYKSLLKITMTQSTVPL | |
| NLIRVHLMVAVEGHLFQKSFQASPNLASTFIWDKTD | |
| AYGQRVYGLSDAVVSVGFEYETCPSLILWEKRTALL | |
| QGFELDPSNLGGWSLDKHHILNVKSGILHKGTGENQ | |
| FLTQQPAIITSIMGNGRRRSISCPSCNGLAEGNKLL | |
| APVALAVGIDGSLYVGDFNYIRRIFPSRNVTSILEL | |
| RNKEFKHSNNPAHKYYLAVDPVSGSLYVSDTNSRRI | |
| YRVKSLSGTKDLAGNSEVVAGTGEQCLPFDEARCGD | |
| GGKAIDATLMSPRGIAVDKNGLMYFVDATMIRKVDQ | |
| NGIISTLLGSNDLTAVRPLSCDSSMDVAQVRLEWPT | |
| DLAVNPMDNSLYVLENNVILRITENHQVSIIAGRPM | |
| HCQVPGIDYSLSKLAIHSALESASAIAISHTGVLYI | |
| TETDEKKINRLRQVTTNGEICLLAGAASDCDCKNDV | |
| NCNCYSGDDAYATDAILNSPSSLAVAPDGTIYIADL | |
| GNIRIRAVSKNKPVLNAFNQYEAASPGEQELYVFNA | |
| DGIHQYTVSLVTGEYLYNFTYSTDNDVTELIDNNGN | |
| SLKIRRDSSGMPRHLLMPDNQIITLTVGTNGGLKVV | |
| STQNLELGLMTYDGNTGLLATKSDETGWTTFYDYDH | |
| EGRLTNVTRPTGVVTSLHREMEKSITIDIENSNRDD | |
| DVTVITNLSSVEASYTVVQDQVRNSYQLCNNGTLRV | |
| MYANGMGISFHSEPHVLAGTITPTIGRCNISLPMEN | |
| GLNSIEWRLRKEQIKGKVTIFGRKLRVHGRNLLSID | |
| YDRNIRTEKIYDDHRKFTLRIIYDQVGRPFLWLPSS | |
| GLAAVNVSYFFNGRLAGLQRGAMSERTDIDKQGRIV | |
| SRMFADGKVWSYSYLDKSMVLLLQSQRQYIFEYDSS | |
| DRLLAVTMPSVARHSMSTHTSIGYIRNIYNPPESNA | |
| SVIFDYSDDGRILKTSFLGTGRQVFYKYGKLSKLSE | |
| IVYDSTAVTFGYDETTGVLKMVNLQSGGFSCTIRYR | |
| KIGPLVDKQIYRFSEEGMVNARFDYTYHDNSFRIAS | |
| IKPVISETPLPVDLYRYDEISGKVEHFGKFGVIYYD | |
| INQIITTAVMTLSKHFDTHGRIKEVQYEMFRSLMYW | |
| MTVQYDSMGRVIKRELKLGPYANTTKYTYDYDGDGQ | |
| LQSVAVNDRPTWRYSYDLNGNLHLLNPGNSVRLMPL | |
| RYDLRDRITRLGDVQYKIDDDGYLCQRGSDIFEYNS | |
| KGLLTRAYNKASGWSVQYRYDGVGRRASYKTNLGHH | |
| LQYFYSDLHNPTRITHVYNHSNSEITSLYYDLQGHL | |
| FAMESSSGEEYYVASDNTGTPLAVFSINGLMIKQLQ | |
| YTAYGEIYYDSNPDFQMVIGFHGGLYDPLTKLVHFT | |
| QRDYDVLAGRWTSPDYTMWKNVGKEPAPFNLYMFKS | |
| NNPLSSELDLKNYVTDVKSWLVMFGFQLSNIIPGFP | |
| RAKMYFVPPPYELSESQASENGQLITGVQQTTERHN | |
| QAFMALEGQVITKKLHASIREKAGHWFATTTPIIGK | |
| GIMFAIKEGRVTTGVSSIASEDSRKVASVLNNAYYL | |
| DKMHYSIEGKDTHYFVKIGSADGDLVTLGTTIGRKV | |
| LESGVNVTVSQPTLLVNGRTRRFTNIEFQYSTLLLS | |
| IRYGLTPDTLDEEKARVLDQARQRALGTAWAKEQQK | |
| ARDGREGSRLWTEGEKQQLLSTGRVQGYEGYYVLPV | |
| EQYPELADSSSNIQFLRQNEMGKR | |

In a BLASTN search it was found that the FCTR3a nucleic acid has homology to three fragments of Mus musculus odd Oz/ten-m homolog 2. It has 634 of 685 bases (92%) identical to bases 614-1298, 365 of 406 bases (89%) identical to bases 1420-1825, and 93 of 103 bases (90%) identical to bases 1823-1925 of Mus musculus odd Oz/ten-m homolog 2 (GenBank Acc: NM_{—}011856.2) (Table 3J).

TABLE 3J
BLASTN of FCTR3a against Mus musculus odd Oz/ten-m homolog 2 (SEQ ID NO:62)

>GI|7657414|REF|NM 011856.2| MUS MUSCULUS ODD OZ/TEN-M HOMOLOG 2 (DROSOPHILA)
(ODZ2), MRNA
LENGTH = 8797
SCORE = 954 BITS (481), EXPECT = 0.0
IDENTITIES = 634/685 (92%)
STRAND = PLUS/PLUS

| | | | |
|---|---|---|---|
| QUERY: | 114 | GGTCGTCCCATTCCACCTACATCCTCGCCTAGTCTCCTCCCATCTGCTCAGCTGCCTAGC | 173 |
| | | \|\|\|\|\|\|\|\|\|\|\|\|\|\|\|\|\|\|\|\|\|\|\|\|\|\|\| \|\|\|\| \|\|\|\|\|\|\|\|\|\|\|\|\|\|\|\|\|\|\|\|\|\|\|\|\|\|\| | |
| SBJCT: | 614 | GGTCGTCCCATTCCACCTACATCCTCGTCTAGCCTCCTCCCATCTGCTCAGCTGCCTAGC | 673 |
| QUERY: | 174 | TCCCATAATCCTCCACCAGTTAGCTGCCAGATGCCATTGCTAGACAGCAACACCTCCCAT | 233 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 674 | TCCCATAATCCTCCACCAGTTAGCTGCCAGATGCCATTGCTAGACAGCAACACCTCCCAT | 733 |
| QUERY: | 234 | CAAATCATGGACACCAACCCTGATGAGGAATTCTCCCCCAATTCATACCTGCTCAGAGCA | 293 |
| | | \|\| \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 734 | CAGATCATGGACACCAACCCTGATGAGGAATTCTCCCCCAATTCATACCTGCTCAGAGCA | 793 |
| QUERY: | 294 | TGCTCAGGGCCCCAGCAAGCCTCCAGCAGTGGCCCTCCGAACCACCACAGCCAGTCGACT | 353 |
| | | \|\|\|\|\|\|\|\|\|\|\|\|\|\|\|\|\|\|\|\|\|\|\|\|\|\|\|\|\|\|\|\|\|\|\|\|\|\| \|\|\|\|\|\|\|\|\|\|\|\|\|\|\|\|\| \|\| | |
| SBJCT: | 794 | TGCTCAGGGCCCCAGCAAGCCTCCAGCAGTGGCCCTCCAAACCACCACAGCCAGTCAACA | 853 |
| QUERY: | 354 | CTGAGGCCCCCTCTCCCACCCCCTCACAACCACACGCTGTCCCATCACCACTCGTCCGCC | 413 |
| | | \|\|\|\|\|\|\|\|\|\|\|\|\|\| \|\|\|\|\|\|\|\|\|\|\| \|\|\|\|\|\|\|\| \|\|\|\|\|\|\|\| \|\|\|\|\|\|\|\| \|\| \|\|\| | |
| SBJCT: | 854 | CTGAGGCCCCCTCTGCCACCCCCTCATAACCACACCCTGTCCCACCACCACTCCTCGGCC | 913 |
| QUERY: | 414 | AACTCCCTCAACAGGAACTCACTGACCAATCGGCGGAGTCAGATCCACGCCCCGGCCCCA | 473 |
| | | \|\|\|\|\|\|\|\|\|\|\|\|\|\|\|\|\|\|\|\|\|\|\|\|\|\|\|\|\|\|\|\|\|\|\|\|\|\|\|\|\| \|\|\|\|\|\|\|\|\|\|\| \|\| \|\| | |
| SBJCT: | 914 | AACTCCCTCAACAGGAACTCACTGACCAATCGGCGGAGTCAAATCCACGCCCCAGCTCCT | 973 |
| QUERY: | 474 | GCGCCCAATGACCTGGCCACCACACCAGAGTCCGTTCAGCTTCAGGACAGCTGGGTGCTA | 533 |
| | | \|\|\|\|\|\|\|\| \|\|\|\|\|\|\|\|\|\|\|\|\|\| \|\|\|\|\|\|\|\| \|\|\|\|\|\|\|\| \|\|\|\|\| \|\|\|\|\|\|\|\|\|\|\| | |
| SBJCT: | 974 | GCGCCCAACGACCTGGCCACCACCCCAGAGTCTGTTCAGCTCCAGGATAGCTGGGTGCTG | 33 |
| QUERY: | 534 | AACAGCAACGTGCCACTGGAGACCCGGCACTTCCTCTTCAAGACCTCCTCGGGGAGCACA | 593 |
| | | \|\|\|\|\| \|\|\|\|\| \|\|\|\|\|\|\|\|\|\|\| \|\|\|\|\|\|\|\|\|\|\| \|\|\|\|\| \|\| \|\| \|\| \|\| \|\|\|\|\|\| | |
| SBJCT: | 1034 | AACAGTAACGTCCCACTGGAGACTCGGCACTTCCTTTTCAAAACGTCGTCTGGAAGCACA | 93 |
| QUERY: | 594 | CCCTTGTTCAGCAGCTCTTCCCCGGGATACCCTTTGACCTCAGGAACGGTTTACACGCCC | 653 |
| | | \|\|\| \|\|\|\|\|\|\|\|\|\|\|\|\|\|\|\| \|\|\|\|\|\|\|\|\|\|\|\|\|\|\|\|\|\|\|\|\|\|\| \|\| \|\|\|\|\| \|\| \|\| | |
| SBJCT: | 1094 | CCCCTGTTCAGCAGCTCTTCTCCGGGATACCCTTTGACCTCAGGGACCGTTTATACACCA | 1153 |
| QUERY: | 654 | CCGCCCCGCCTGCTGCCCAGGAATACTTTCTCCAGGAAGGCTTTCAAGCTGAAGAAGCCC | 713 |
| | | \|\| \|\|\|\|\|\|\|\|\|\|\|\|\|\| \|\|\|\|\|\|\| \|\|\|\|\|\|\|\|\|\|\|\|\|\| \|\|\|\|\|\|\|\|\|\|\|\|\|\|\| \|\| | |
| SBJCT: | 1154 | CCACCCCGCCTGCTGCCACGGAATACATTCTCCAGGAAGGCCTTCAAGCTGAAGAAACCC | 1213 |
| QUERY: | 714 | TCCAAATACTGCAGCTGGAAATGTGCTGCCCTCTCCGCCATTGCCGCGGCCCTCCTCTTG | 773 |
| | | \|\|\|\|\|\|\|\|\|\|\|\|\|\| \|\|\|\|\|\|\|\|\|\|\|\|\|\|\|\|\| \|\| \|\|\|\|\| \|\|\|\|\| \|\|\|\|\|\|\|\|\|\|\|\| | |
| SBJCT: | 1214 | TCCAAATACTGCAGTTGGAAATGTGCTGCCCTGTCTGCCATCGCCGCCGCCCTCCTCTTG | 1273 |
| QUERY: | 774 | GCTATTTTGCTGGCGTATTTCATAG 798 | |
| | | \|\| \|\|\|\|\|\|\|\|\|\|\| \|\|\|\|\|\|\|\|\|\| | |
| SBJCT: | 1274 | GCCATTTTGCTGGCATATTTCATAG 1298 | |

SCORE = 480 BITS (242), EXPECT = E-132
IDENTITIES = 365/406 (89%)
STRAND = PLUS/PLUS

| | | | |
|---|---|---|---|
| QUERY: | 797 | AGTGCCCTGGTCGTTGAAAAACAGCAGCATAGACAGTGGTGAAGCAGAAGTTGGTCGGCG | 856 |
| | | \|\|\|\|\|\|\|\|\|\|\|\| \|\|\|\|\|\|\|\|\|\|\|\|\|\|\|\|\|\|\|\|\|\|\|\|\|\| \|\|\|\|\|\|\|\|\|\|\|\|\|\|\|\|\|\|\|\| | |
| SBJCT: | 1420 | AGTGCCCTGGTCATTGAAAAACAGCAGCATAGACAGTGGCGAAGCAGAAGTTGGTCGGCG | 1479 |
| QUERY: | 857 | GGTAACACAAGAAGTCCCACCAGGGGTGTTTTGGAGGTCACAAATTCACATCAGTCAGCC | 916 |
| | | \|\|\| \|\|\|\|\| \|\|\|\|\|\|\|\|\|\|\|\|\|\|\|\|\|\|\|\|\|\|\|\|\|\|\|\|\| \|\| \|\|\|\|\|\|\|\|\|\|\|\|\|\|\|\|\| | |
| SBJCT: | 1480 | GGTGACACAGGAAGTCCCACCAGGGGTGTTTTGGAGGTCCCAGATTCACATCAGTCAGCC | 1539 |
| QUERY: | 917 | CCAGTTCTTAAAGTTCAACATCTCCCTCGGGAAGGACGCTCTCTTTGGTGTTTACATAAG | 976 |
| | | \|\| \|\|\|\|\|\|\|\|\|\|\|\|\|\|\|\|\|\|\|\|\|\|\| \|\| \|\|\|\|\| \|\| \|\|\|\|\| \|\|\|\|\| \|\| \|\|\|\|\| | |
| SBJCT: | 1540 | TCAATTCTTAAAGTTCAACATCTCCCTGGGCAAGGATGCCCTCTTCGGTGTCTATATAAG | 1599 |
| QUERY: | 977 | AAGAGGACTTCCACCATCTCATGCCCAGTATGACTTCATGGAACGTCTGGACGGGAAGGA | 36 |
| | | \|\|\|\|\|\|\|\| \|\|\|\|\| \|\|\|\|\|\|\|\|\|\|\|\|\|\|\|\|\|\|\|\|\|\|\|\|\|\|\|\|\| \|\|\|\|\| \|\| \|\|\|\|\| | |
| SBJCT: | 1600 | GAGAGGACTACCACCGTCTCATGCCCAGTATGACTTCATGGAACGCCTGGATGGAAAGGA | 1659 |
| QUERY: | 1037 | GAAGTGGAGTGTGGTTGAGTCTCCCAGGGAACGCCGGAGCATACAGACCTTGGTTCAGAA | 96 |
| | | \|\|\| \|\|\|\|\| \|\|\|\|\| \|\|\|\|\| \|\|\|\|\|\|\|\|\|\|\|\|\|\|\|\|\|\|\|\| \|\|\|\|\| \|\|\|\| \|\|\|\|\| | |
| SBJCT: | 1660 | GAAATGGAGCGTGGTCGAGTCGCCCAGGGAACGCCGGAGCATCCAGACTCTGGTGCAGAA | 1719 |
| QUERY: | 1097 | TGAAGCCGTGTTTGTGCAGTACCTGGATGTGGGCCTGTGGCATCTGGCCTTCTACAATGA | 1156 |
| | | \|\| \|\| \|\|\|\|\|\|\|\|\|\|\|\|\|\|\| \|\|\|\|\|\|\|\|\|\|\|\|\|\|\|\|\|\|\| \|\|\|\|\|\|\|\|\|\|\|\|\|\|\|\|\| | |
| SBJCT: | 1720 | CGAGGCTGTGTTTGTGCAGTACTTGGATGTGGGCCTGTGGCACCTGGCCTTCTACAATGA | 1779 |
| QUERY: | 1157 | TGGAAAAGACAAAGAGATGGTTTCCTTCAATACTGTTGTCCTAGAT 1202 | |
| | | \|\| \|\| \|\|\|\|\| \|\|\|\|\|\|\|\| \|\|\|\|\|\|\|\| \|\|\|\|\|\|\|\|\| \|\|\|\|\| | |
| SBJCT: | 1780 | CGGCAAGGACAAGGAGATGGTCTCCTTCAACACTGTTGTCTTAGAT 1825 | |

SCORE = 125 BITS (63), EXPECT = 7E-26
IDENTITIES = 93/ 3 (90%)
STRAND = PLUS/PLUS

| | | | |
|---|---|---|---|
| QUERY: | 1258 | GATTCAGTGCAGGACTGTCCACGTAACTGCCATGGGAATGGTGAATGTGTGTCCGGGGTG | 1317 |
| | | \|\|\|\|\|\|\|\|\|\|\|\|\|\|\|\|\|\|\|\|\|\|\| \|\|\|\|\| \|\| \|\|\|\|\| \|\|\|\|\|\|\|\| \|\|\|\|\| \|\| \|\| | |
| SBJCT: | 1823 | GATTCAGTGCAGGACTGTCCACGGAACTGTCACGGGAACGGTGAATGCGTGTCTGGACTG | 1882 |
| QUERY: | 1318 | TGTCACTGTTTCCCAGGATTTCTAGGAGCAGACTGTGCTAAAG 1360 | |
| | | \|\|\|\|\|\|\|\|\|\|\|\|\|\|\|\|\|\|\|\| \|\|\|\|\| \|\|\|\|\|\|\|\|\|\|\|\|\|\|\|\| | |
| SBJCT: | 1883 | TGTCACTGTTTCCCAGGATTCCTAGGTGCAGACTGTGCTAAAG 1925 | |

In another BLASTN search it was found that the FCTR3a nucleic acid has homology to three fragments of Gallus gallus mRNA for teneurin-2. It has 541 of 629 bases (86%) identical to bases 502-1130, 302 of 367 bases (82%) identical to bases 1330-1696, and 87 of 103 bases (84%) identical to bases 1711-1813 of Gallus gallus mRNA for teneurin-2 (EMBL Acc: AJ245711.1) (Table 3K).

TABLE 3K
BLASTN of FCTR3a against Gallus gallus mRNA for teneurin-2 (SEQ ID NO:63)

>GI|60l0048|EMB|AJ245711.1|GGA245711 GALLUS GALLUS MRNA FOR TENEURIN-2, SHORT
SPLICE VARIANT (TEN2 GENE)
LENGTH = 2496
SCORE = 549 BITS (277), EXPECT = E-153
IDENTITIES = 541/629 (86%)
STRAND = PLUS/PLUS

| | | | |
|---|---|---|---|
| QUERY: | 114 | GGTCGTCCCATTCCACCTACATCCTCGCCTAGTCTCCTCCCATCTGCTCAGCTGCCTAGC | 173 |
| | | \|\|\|\|\|\|\|\|\|\|\|\|\|\|\|\|\|\|\|\|\|\|\|\|\|\|\| \|\|\|\| \|\| \|\|\|\|\|\|\|\|\|\|\|\|\|\|\|\|\|\|\|\| \|\| | |
| SBJCT: | 502 | GGTCGTCCCATTCCACCTACATCCTCGTCTAGCCTTCTCCCATCTGCTCAGCTGCCCAGT | 561 |
| QUERY: | 174 | TCCCATAATCCTCCACCAGTTAGCTGCCAGATGCCATTGCTAGACAGCAACACCTCCCAT | 233 |
| | | \|\| \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| \|\| \|\|\|\|\|\| | |
| SBJCT: | 562 | TCTCATAATCCTCCACCAGTTAGCTGCCAGATGCCATTGCTAGACAGCAATACGTCCCAT | 621 |
| QUERY: | 234 | CAAATCATGGACACCAACCCTGATGAGGAATTCTCCCCCAATTCATACCTGCTCAGAGCA | 293 |
| | | \|\|\|\|\|\|\|\|\|\|\|\|\|\|\|\|\| \|\|\|\|\| \|\|\|\|\| \|\|\|\|\| \|\| \|\|\|\|\|\|\|\|\|\|\| \|\| \|\|\|\|\|\| | |
| SBJCT: | 622 | CAAATCATGGACACCAATCCTGACGAGGAGTTCTCTCCTAATTCATACCTACTAAGAGCA | 681 |
| QUERY: | 294 | TGCTCAGGGCCCCAGCAAGCCTCCAGCAGTGGCCCTCCGAACCACCACAGCCAGTCGACT | 353 |
| | | \|\| \|\|\|\|\|\|\|\| \|\|\|\|\| \|\| \|\|\|\|\|\|\|\|\|\|\|\|\|\|\| \| \|\|\|\|\| \|\|\|\|\|\|\|\|\|\|\| \|\| | |
| SBJCT: | 682 | TGTTCAGGGCCACAGCAGGCATCCAGCAGTGGCCCTTCAAACCATCACAGCCAGTCAACG | 741 |
| QUERY: | 354 | CTGAGGCCCCCTCTCCCACCCCCTCACAACCACACGCTGTCCCATCACCACTCGTCCGCC | 413 |
| | | \|\|\|\|\|\|\|\| \|\|\|\|\|\|\|\| \|\| \|\|\|\|\|\|\|\|\|\|\|\| \|\|\|\|\|\|\|\|\|\|\|\|\| \|\|\|\|\|\|\|\| \|\|\| | |
| SBJCT: | 742 | CTGAGGCCACCTCTCCCCCCTCCTCACAACCACTCGCTGTCCCATCATCACTCGTCTGCC | 801 |
| QUERY: | 414 | AACTCCCTCAACAGGAACTCACTGACCAATCGGCGGAGTCAGATCCACGCCCCGGCCCCA | 473 |
| | | \|\|\|\|\|\|\|\|\|\|\|\|\|\|\|\|\|\|\|\| \|\| \|\|\|\|\| \|\| \|\| \| \|\|\|\|\|\|\|\|\|\|\| \|\| \|\| \|\| | |
| SBJCT: | 802 | AACTCCCTCAACAGGAACTCGCTCACCAACCGCCGCAACCAGATCCACGCGCCTGCTCCC | 861 |
| QUERY: | 474 | GCGCCCAATGACCTGGCCACCACACCAGAGTCCGTTCAGCTTCAGGACAGCTGGGTGCTA | 533 |
| | | \|\| \|\|\|\|\|\|\|\|\|\|\|\|\|\| \|\|\|\|\| \|\| \|\|\|\|\| \|\| \|\|\|\|\| \|\|\|\|\|\|\|\|\|\|\|\|\|\|\|\|\| | |
| SBJCT: | 862 | GCTCCCAATGACCTGGCGACCACGCCTGAGTCTGTGCAGCTGCAGGACAGCTGGGTGCTC | 921 |
| QUERY: | 534 | AACAGCAACGTGCCACTGGAGACCCGGCACTTCCTCTTCAAGACCTCCTCGGGGAGCACA | 593 |
| | | \|\|\|\|\|\|\|\|\|\|\|\|\|\| \|\|\|\|\|\|\|\|\| \|\|\|\| \|\|\| \| \|\| \|\|\|\|\| \|\| \|\| \|\| \| \|\| | |
| SBJCT: | 922 | AACAGCAACGTGCCGCTGGAGACCAGGCATTTCTTGTTTAAGACATCTTCTGGAACGACT | 981 |
| QUERY: | 594 | CCCTTGTTCAGCAGCTCTTCCCCGGGATACCCTTTGACCTCAGGAACGGTTTACACGCCC | 653 |
| | | \|\| \|\|\|\|\|\|\| \|\|\|\|\|\|\|\|\|\|\| \|\| \|\|\|\|\| \|\|\|\|\|\|\|\|\|\|\|\|\| \|\|\|\|\| \|\| \|\| | |
| SBJCT: | 982 | CCGCTGTTCAGTAGCTCTTCCCCTGGCTACCCACTGACCTCAGGAACAGTTTATACTCCA | 1041 |
| QUERY: | 654 | CCGCCCCGCCTGCTGCCCAGGAATACTTTCTCCAGGAAGGCTTTCAAGCTGAAGAAGCCC | 713 |
| | | \|\| \|\|\| \| \|\|\| \| \|\| \|\| \|\|\|\|\| \|\| \|\|\|\|\|\|\|\| \|\| \|\|\|\|\|\|\|\|\|\|\| \|\|\|\|\|\| | |
| SBJCT: | 1042 | CCTCCCAGGCTGTTACCTAGAAATACATTTTCCAGGAATGCATTCAAGCTGAAAAAGCCC | 1101 |
| QUERY: | 714 | TCCAAATACTGCAGCTGGAAATGTGCTGC 742 | |
| | | \|\|\|\|\| \|\| \|\| \|\|\|\|\|\|\|\|\|\|\|\|\|\|\|\|\| | |
| SBJCT: | 1102 | TCCAAGTATTGTAGCTGGAAATGTGCTGC 1130 | |

SCORE = 212 BITS (107), EXPECT = 4E-52
IDENTITIES = 302/367 (82%)
STRAND = PLUS/PLUS

| | | | |
|---|---|---|---|
| QUERY: | 819 | AGCAGCATAGACAGTGGTGAAGCAGAAGTTGGTCGGCGGGTAACACAAGAAGTCCCACCA | 878 |
| | | \|\|\|\|\|\|\|\|\|\|\| \|\|\|\|\| \|\|\| \|\|\|\|\|\|\|\|\|\| \|\| \|\|\| \|\| \|\|\|\|\| \|\| \|\| \|\| | |
| SBJCT: | 1330 | AGCAGCATAGATAGTGGAGAAACAGAAGTTGGCCGCAAGGTCACCCAAGAGGTGCCCCCT | 1389 |
| QUERY: | 879 | GGGGTGTTTTGGAGGTCACAAATTCACATCAGTCAGCCCCAGTTCTTAAAGTTCAACATC | 938 |
| | | \|\| \|\|\|\|\| \|\|\| \|\|\|\| \|\| \|\| \|\| \|\|\|\|\| \|\|\|\|\| \|\|\|\|\|\| \| \|\|\|\|\|\|\|\|\|\|\| | |
| SBJCT: | 1390 | GGAGTGTTCTGGCGGTCTCAGATCCATATCAGCCAGCCACAGTTCCTGAAGTTCAACATA | 1449 |
| QUERY: | 939 | TCCCTCGGGAAGGACGCTCTCTTTGGTGTTTACATAAGAAGAGGACTTCCACCATCTCAT | 998 |
| | | \|\|\|\|\| \|\|\|\|\|\|\|\| \|\|\|\|\| \|\| \|\|\|\|\|\|\|\| \|\|\|\|\|\|\|\|\|\|\|\|\|\| \|\|\|\|\|\|\|\| \|\|\| | |
| SBJCT: | 1450 | TCCCTAGGGAAGGATGCTCTTTTCGGTGTTTATATAAGAAGAGGACTCCCACCATCACAT | 1509 |
| QUERY: | 999 | GCCCAGTATGACTTCATGGAACGTCTGGACGGGAAGGAGAAGTGGAGTGTGGTTGAGTCT | 1058 |
| | | \|\| \|\|\|\|\|\|\|\| \|\|\|\|\|\|\|\|\|\|\| \|\|\|\| \|\|\|\|\| \|\|\|\|\| \|\|\|\|\|\|\|\|\|\|\| \|\| \|\| | |
| SBJCT: | 1510 | GCACAGTATGATTTCATGGAACGCTTGGATGGGAAAGAGAAATGGAGTGTGGTGGAATCC | 1569 |
| QUERY: | 1059 | CCCAGGGAACGCCGGAGCATACAGACCTTGGTTCAGAATGAAGCCGTGTTTGTGCAGTAC | 1118 |
| | | \|\| \|\|\|\|\|\|\| \|\| \|\| \|\| \|\|\|\|\| \| \|\|\|\|\|\|\|\|\|\|\| \|\| \|\|\|\|\|\|\|\| \|\|\|\|\|\| | |
| SBJCT: | 1570 | CCACGGGAACGGCGAAGTATTCAGACTCTTGTTCAGAATGAGGCTGTGTTTGTTCAGTAC | 1629 |
| QUERY: | 1119 | CTGGATGTGGGCCTGTGGCATCTGGCCTTCTACAATGATGGAAAAGACAAAGAGATGGTT | 1178 |
| | | \|\|\|\|\|\|\|\|\|\| \|\|\|\|\|\|\| \|\|\|\|\| \|\| \|\|\|\|\|\|\|\|\|\|\| \|\| \|\|\|\|\|\|\|\| \|\|\|\| | |
| SBJCT: | 1630 | TTGGATGTGGGTTTGTGGCACCTGGCGTTTTACAATGATGGCAAGGACAAAGAAGTGGTC | 1689 |
| QUERY: | 1179 | TCCTTCA 1185 | |
| | | \|\|\|\|\|\|\| | |
| SBJCT: | 1690 | TCCTTCA 1696 | |

SCORE = 77.8 BITS (39), EXPECT = 1E-11
IDENTITIES = 87/103 (84%)
STRAND = PLUS/PLUS

| | | | |
|---|---|---|---|
| QUERY: | 1258 | GATTCAGTGCAGGACTGTCCACGTAACTGCCATGGGAATGGTGAATGTGTGTCCGGGGTG | 1317 |
| | | \|\|\|\|\|\|\|\|\|\|\| \|\|\|\|\|\|\|\|\|\|\|\|\|\| \|\| \|\|\|\|\| \|\|\|\|\| \|\| \|\|\|\|\| \|\| \|\| \|\| | |
| SBJCT: | 1711 | GATTCAGTGCAAGACTGTCCACGTAATTGTCATGGCAATGGCGAGTGTGTTTCTGGTGTC | 1770 |
| QUERY: | 1318 | TGTCACTGTTTCCCAGGATTTCTAGGAGCAGACTGTGCTAAAG 1360 | |
| | | \|\| \|\|\|\|\|\|\|\| \|\| \|\|\|\|\|\|\| \|\|\|\|\|\|\|\| \|\|\|\|\|\|\|\|\|\| | |
| SBJCT: | 1771 | TGCCACTGTTTTCCCGGATTTCATGGAGCAGATTGTGCTAAAG 1813 | |

In this search it was also found that the fragments of FCTR3bcd and e nucleic acids had homology to three fragments of Homo sapiens mRNA for KIAA1127 protein. It has 5537 of 5538 bases (99%) identical to bases 1-5538, 705 of 714 bases (98%) identical to bases 5609-6322, and 176 of 176 bases (100%) identical to bases 6385-6560 of Homo sapiens mRNA for KIAA1 127 protein (GenBank Acc: AB032953) (Table 3L).

TABLE 3L

BLASTN of FCTR3b, c, d, and e against Homo sapiens KIAA1127 mRNA (SEQ ID NO:
64)

>GI|6329762|AB032953.1|AB032953 HOMO SAPIENS MRNA FOR KIAA1127 PROTEIN,
PARTIAL CDS
LENGTH = 6560
SCORE = 1.097E+04 BITS (5534), EXPECT = 0.0
IDENTITIES = 5537/5538 (99%)
STRAND = PLUS/PLUS

| | | | |
|---|---|---|---|
| QUERY: | 3267 | CACCTTCTTTAGTGCTGCCCCTGGGCAGAATCCCATCGTGCCTGAGACCCAGGTTCTTCA | 3326 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 1 | CACCTTCTTTAGTGCTGCCCCTGGGCAGAATCCCATCGTGCCTGAGACCCAGGTTCTTCA | 60 |
| QUERY: | 3327 | TGAAGAAATCGAGCTCCCTGGTTCCAATGTGAAACTTCGCTATCTGAGCTCTAGAACTGC | 3386 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 61 | TGAAGAAATCGAGCTCCCTGGTTCCAATGTGAAACTTCGCTATCTGAGCTCTAGAACTGC | 120 |
| QUERY: | 3387 | AGGGTACAAGTCACTGCTGAAGATCACCATGACCCAGTCCACAGTGCCCCTGAACCTCAT | 3446 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 121 | AGGGTACAAGTCACTGCTGAAGATCACCATGACCCAGTCCACAGTGCCCCTGAACCTCAT | 180 |
| QUERY: | 3447 | TAGGGTTCACCTGATGGTGGCTGTCGAGGGGCATCTCTTCCAGAAGTCATTCCAGGCTTC | 3506 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 181 | TAGGGTTCACCTGATGGTGGCTGTCGAGGGGCATCTCTTCCAGAAGTCATTCCAGGCTTC | 240 |
| QUERY: | 3507 | TCCCAACCTGGCCTCCACCTTCATCTGGGACAAGACAGATGCGTATGGCCAAAGGGTGTA | 3566 |
| | | \|\|\|\|\|\|\|\|\|\|\|\|\|\| \|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 241 | TCCCAACCTGGCCTACACCTTCATCTGGGACAAGACAGATGCGTATGGCCAAAGGGTGTA | 300 |
| QUERY: | 3567 | TGGACTCTCAGATGCTGTTGTGTCTGTCGGGTTTGAATATGAGACCTGTCCCAGTCTAAT | 3626 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 301 | TGGACTCTCAGATGCTGTTGTGTCTGTCGGGTTTGAATATGAGACCTGTCCCAGTCTAAT | 360 |
| QUERY: | 3627 | TCTCTGGGAGAAAAGGACAGCCCTCCTTCAGGGATTCGAGCTGGACCCCTCCAACCTCGG | 3686 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 361 | TCTCTGGGAGAAAAGGACAGCCCTCCTTCAGGGATTCGAGCTGGACCCCTCCAACCTCGG | 420 |
| QUERY: | 3687 | TGGCTGGTCCCTAGACAAACACCACATCCTCAATGTTAAAAGTGGAATCCTACACAAAGG | 3746 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 421 | TGGCTGGTCCCTAGACAAACACCACATCCTCAATGTTAAAAGTGGAATCCTACACAAAGG | 480 |
| QUERY: | 3747 | CACTGGGGAAAACCAGTTCCTGACCCAGCAGCCTGCCATCATCACCAGCATCATGGGCAA | 3806 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 481 | CACTGGGGAAAACCAGTTCCTGACCCAGCAGCCTGCCATCATCACCAGCATCATGGGCAA | 540 |
| QUERY: | 3807 | TGGTCGCCGCCGGAGCATTTCCTGTCCCAGCTGCAACGGCCTTGCTGAAGGCAACAAGCT | 3866 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 541 | TGGTCGCCGCCGGAGCATTTCCTGTCCCAGCTGCAACGGCCTTGCTGAAGGCAACAAGCT | 600 |
| QUERY: | 3867 | GCTGGCCCCAGTGGCTCTGGCTGTTGGAATCGATGGGAGCCTCTATGTGGGTGACTTCAA | 3926 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 601 | GCTGGCCCCAGTGGCTCTGGCTGTTGGAATCGATGGGAGCCTCTATGTGGGTGACTTCAA | 660 |
| QUERY: | 3927 | TTACATCCGACGCATCTTTCCCTCTCGAAATGTGACCAGCATCTTGGAGTTACGAAATAA | 3986 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 661 | TTACATCCGACGCATCTTTCCCTCTCGAAATGTGACCAGCATCTTGGAGTTACGAAATAA | 720 |
| QUERY: | 3987 | AGAGTTTAAACATAGCAACAACCCAGCACACAAGTACTACTTGGCAGTGGACCCCGTGTC | 4046 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 721 | AGAGTTTAAACATAGCAACAACCCAGCACACAAGTACTACTTGGCAGTGGACCCCGTGTC | 780 |
| QUERY: | 4047 | CGGCTCGCTCTACGTGTCCGACACCAACAGCAGGAGAATCTACCGCGTCAAGTCTCTGAG | 4106 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 781 | CGGCTCGCTCTACGTGTCCGACACCAACAGCAGGAGAATCTACCGCGTCAAGTCTCTGAG | 840 |
| QUERY: | 4107 | TGGAACCAAAGACCTGGCTGGGAATTCGGAAGTTGTGGCAGGGACGGGAGAGCAGTGTCT | 4166 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 841 | TGGAACCAAAGACCTGGCTGGGAATTCGGAAGTTGTGGCAGGGACGGGAGAGCAGTGTCT | 900 |
| QUERY: | 4167 | ACCCTTTGATGAAGCCCGCTGCGGGGATGGAGGGAAGGCCATAGATGCAACCCTGATGAG | 4226 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 901 | ACCCTTTGATGAAGCCCGCTGCGGGGATGGAGGGAAGGCCATAGATGCAACCCTGATGAG | 960 |
| QUERY: | 4227 | CCCGAGAGGTATTGCAGTAGACAAGAATGGGCTCATGTACTTTGTCGATGCCACCATGAT | 4286 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 961 | CCCGAGAGGTATTGCAGTAGACAAGAATGGGCTCATGTACTTTGTCGATGCCACCATGAT | 1020 |
| QUERY: | 4287 | CCGGAAGGTTGACCAGAATGGAATCATCTCCACCCTGCTGGGCTCCAATGACCTCACTGC | 4346 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 1021 | CCGGAAGGTTGACCAGAATGGAATCATCTCCACCCTGCTGGGCTCCAATGACCTCACTGC | 1080 |
| QUERY: | 4347 | CGTCCGGCCGCTGAGCTGTGATTCCAGCATGGATGTAGCCCAGGTTCGTCTGGAGTGGCC | 4406 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 1081 | CGTCCGGCCGCTGAGCTGTGATTCCAGCATGGATGTAGCCCAGGTTCGTCTGGAGTGGCC | 1140 |
| QUERY: | 4407 | AACAGACCTTGCTGTCAATCCCATGGATAACTCCTTGTATGTTCTAGAGAACAATGTCAT | 4466 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 1141 | AACAGACCTTGCTGTCAATCCCATGGATAACTCCTTGTATGTTCTAGAGAACAATGTCAT | 1200 |
| QUERY: | 4467 | CCTTCGAATCACCGAGAACCACCAAGTCAGCATCATTGCGGGACGCCCCATGCACTGCCA | 4526 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 1201 | CCTTCGAATCACCGAGAACCACCAAGTCAGCATCATTGCGGGACGCCCCATGCACTGCCA | 1260 |
| QUERY: | 4527 | AGTTCCTGGCATTGACTACTCACTCAGCAAACTAGCCATTCACTCTGCCCTGGAGTCAGC | 4586 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 1261 | AGTTCCTGGCATTGACTACTCACTCAGCAAACTAGCCATTCACTCTGCCCTGGAGTCAGC | 1320 |
| QUERY: | 4587 | CAGTGCCATTGCCATTTCTCACACTGGGGTCCTCTACATCACTGAGACAGATGAGAAGAA | 4646 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 1321 | CAGTGCCATTGCCATTTCTCACACTGGGGTCCTCTACATCACTGAGACAGATGAGAAGAA | 1380 |
| QUERY: | 4647 | GATTAACCGTCTACGCCAGGTAACAACCAACGGGGAGATCTGCCTTTTAGCTGGGGCAGC | 4706 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 1381 | GATTAACCGTCTACGCCAGGTAACAACCAACGGGGAGATCTGCCTTTTAGCTGGGGCAGC | 1440 |
| QUERY: | 4707 | CTCGGACTGCGACTGCAAAAACGATGTCAATTGCAACTGCTATTCAGGAGATGATGCCTA | 4766 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 1441 | CTCGGACTGCGACTGCAAAAACGATGTCAATTGCAACTGCTATTCAGGAGATGATGCCTA | 1500 |
| QUERY: | 4767 | CGCGACTGATGCCATCTTGAATTCCCCATCATCCTTAGCTGTAGCTCCAGATGGTACCAT | 4826 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 1501 | CGCGACTGATGCCATCTTGAATTCCCCATCATCCTTAGCTGTAGCTCCAGATGGTACCAT | 1560 |
| QUERY: | 4827 | TTACATTGCAGACCTTGGAAATATTCGGATCAGGGCGGTCAGCAAGAACAAGCCTGTTCT | 4886 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 1561 | TTACATTGCAGACCTTGGAAATATTCGGATCAGGGCGGTCAGCAAGAACAAGCCTGTTCT | 1620 |
| QUERY: | 4887 | TAATGCCTTCAACCAGTATGAGGCTGCATCCCCCGGAGAGCAGGAGTTATATGTTTTCAA | 4946 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 1621 | TAATGCCTTCAACCAGTATGAGGCTGCATCCCCCGGAGAGCAGGAGTTATATGTTTTCAA | 1680 |
| QUERY: | 4947 | CGCTGATGGCATCCACCAATACACTGTGAGCCTGGTGACAGGGGAGTACTTGTACAATTT | 5006 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 1681 | CGCTGATGGCATCCACCAATACACTGTGAGCCTGGTGACAGGGGAGTACTTGTACAATTT | 1740 |
| QUERY: | 5007 | CACATATAGTACTGACAATGATGTCACTGAATTGATTGACAATAATGGGAATTCCCTGAA | 5066 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 1741 | CACATATAGTACTGACAATGATGTCACTGAATTGATTGACAATAATGGGAATTCCCTGAA | 1800 |
| QUERY: | 5067 | GATCCGTCGGGACAGCAGTGGCATGCCCCGTCACCTGCTCATGCCTGACAACCAGATCAT | 5126 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 1801 | GATCCGTCGGGACAGCAGTGGCATGCCCCGTCACCTGCTCATGCCTGACAACCAGATCAT | 1860 |
| QUERY: | 5127 | CACCCTCACCGTGGGCACCAATGGAGGCCTCAAAGTCGTGTCCACACAGAACCTGGAGCT | 5186 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 1861 | CACCCTCACCGTGGGCACCAATGGAGGCCTCAAAGTCGTGTCCACACAGAACCTGGAGCT | 1920 |
| QUERY: | 5187 | TGGTCTCATGACCTATGATGGCAACACTGGGCTCCTGGCCACCAAGAGCGATGAAACAGG | 5246 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 1921 | TGGTCTCATGACCTATGATGGCAACACTGGGCTCCTGGCCACCAAGAGCGATGAAACAGG | 1980 |
| QUERY: | 5247 | ATGGACGACTTTCTATGACTATGACCACGAAGGCCGCCTGACCAACGTGACGCGCCCCAC | 5306 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 1981 | ATGGACGACTTTCTATGACTATGACCACGAAGGCCGCCTGACCAACGTGACGCGCCCCAC | 2040 |
| QUERY: | 5307 | GGGGGTGGTAACCAGTCTGCACCGGGAAATGGAGAAATCTATTACCATTGACATTGAGAA | 5366 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 2041 | GGGGGTGGTAACCAGTCTGCACCGGGAAATGGAGAAATCTATTACCATTGACATTGAGAA | 2100 |
| QUERY: | 5367 | CTCCAACCGTGATGATGACGTCACTGTCATCACCAACCTCTCTTCAGTAGAGGCCTCCTA | 5426 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 2101 | CTCCAACCGTGATGATGACGTCACTGTCATCACCAACCTCTCTTCAGTAGAGGCCTCCTA | 2160 |
| QUERY: | 5427 | CACAGTGGTACAAGATCAAGTTCGGAACAGCTACCAGCTCTGTAATAATGGTACCCTGAG | 5486 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 2161 | CACAGTGGTACAAGATCAAGTTCGGAACAGCTACCAGCTCTGTAATAATGGTACCCTGAG | 2220 |
| QUERY: | 5487 | GGTGATGTATGCTAATGGGATGGGTATCAGCTTCCACAGCGAGCCCCATGTCCTAGCGGG | 5546 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 2221 | GGTGATGTATGCTAATGGGATGGGTATCAGCTTCCACAGCGAGCCCCATGTCCTAGCGGG | 2280 |
| QUERY: | 5547 | CACCATCACCCCCACCATTGGACGCTGCAACATCTCCCTGCCTATGGAGAATGGCTTAAA | 5606 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 2281 | CACCATCACCCCCACCATTGGACGCTGCAACATCTCCCTGCCTATGGAGAATGGCTTAAA | 2340 |
| QUERY: | 6627 | TGGGCGGATCAAGGAGGTCCAGTATGAGATGTTCCGGTCCCTCATGTACTGGATGACGGT | 6686 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 3361 | TGGGCGGATCAAGGAGGTCCAGTATGAGATGTTCCGGTCCCTCATGTACTGGATGACGGT | 3420 |
| QUERY: | 6687 | GCAATATGACAGCATGGGCAGGGTGATCAAGAGGGAGCTAAAACTGGGGCCCTATGCCAA | 6746 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 3421 | GCAATATGACAGCATGGGCAGGGTGATCAAGAGGGAGCTAAAACTGGGGCCCTATGCCAA | 3480 |
| QUERY: | 6747 | TACCACGAAGTACACCTATGACTACGATGGGGACGGGCAGCTCCAGAGCGTGGCCGTCAA | 6806 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 3481 | TACCACGAAGTACACCTATGACTACGATGGGGACGGGCAGCTCCAGAGCGTGGCCGTCAA | 3540 |
| QUERY: | 6807 | TGACCGCCCGACCTGGCGCTACAGCTATGACCTTAATGGGAATCTCCACTTACTGAACCC | 6866 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 3541 | TGACCGCCCGACCTGGCGCTACAGCTATGACCTTAATGGGAATCTCCACTTACTGAACCC | 3600 |
| QUERY: | 6867 | AGGCAACAGTGTGCGCCTCATGCCCTTGCGCTATGACCTCCGGGATCGGATAACCAGACT | 6926 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 3601 | AGGCAACAGTGTGCGCCTCATGCCCTTGCGCTATGACCTCCGGGATCGGATAACCAGACT | 3660 |
| QUERY: | 6927 | CGGGGATGTGCAGTACAAAATTGACGACGATGGCTATCTGTGCCAGAGAGGGTCTGACAT | 6986 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 3661 | CGGGGATGTGCAGTACAAAATTGACGACGATGGCTATCTGTGCCAGAGAGGGTCTGACAT | 3720 |
| QUERY: | 6987 | CTTCGAATACAATTCCAAGGGCCTCCTAACAAGAGCCTACAACAAGGCCAGCGGGTGGAG | 7046 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 3721 | CTTCGAATACAATTCCAAGGGCCTCCTAACAAGAGCCTACAACAAGGCCAGCGGGTGGAG | 3780 |
| QUERY: | 7047 | TGTCCAGTACCGCTATGATGGCGTAGGACGGCGGGCTTCCTACAAGACCAACCTGGGCCA | 7106 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 3781 | TGTCCAGTACCGCTATGATGGCGTAGGACGGCGGGCTTCCTACAAGACCAACCTGGGCCA | 3840 |
| QUERY: | 7107 | CCACCTGCAGTACTTCTACTCTGACCTCCACAACCCGACGCGCATCACCCATGTCTACAA | 7166 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 3841 | CCACCTGCAGTACTTCTACTCTGACCTCCACAACCCGACGCGCATCACCCATGTCTACAA | 3900 |
| QUERY: | 7167 | TCACTCCAACTCGGAGATTACCTCACTGTACTACGACCTCCAGGGCCACCTCTTTGCCAT | 7226 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 3901 | TCACTCCAACTCGGAGATTACCTCACTGTACTACGACCTCCAGGGCCACCTCTTTGCCAT | 3960 |
| QUERY: | 7227 | GGAGAGCAGCAGTGGGGAGGAGTACTATGTTGCCTCTGATAACACAGGGACTCCTCTGGC | 7286 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 3961 | GGAGAGCAGCAGTGGGGAGGAGTACTATGTTGCCTCTGATAACACAGGGACTCCTCTGGC | 4020 |
| QUERY: | 7287 | TGTGTTCAGCATCAACGGCCTCATGATCAAACAGCTGCAGTACACGGCCTATGGGGAGAT | 7346 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 4021 | TGTGTTCAGCATCAACGGCCTCATGATCAAACAGCTGCAGTACACGGCCTATGGGGAGAT | 4080 |
| QUERY: | 7347 | TTATTATGACTCCAACCCCGACTTCCAGATGGTCATTGGCTTCCATGGGGGACTCTATGA | 7406 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 4081 | TTATTATGACTCCAACCCCGACTTCCAGATGGTCATTGGCTTCCATGGGGGACTCTATGA | 4140 |
| QUERY: | 7407 | CCCCCTGACCAAGCTGGTCCACTTCACTCAGCGTGATTATGATGTGCTGGCAGGACGATG | 7466 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 4141 | CCCCCTGACCAAGCTGGTCCACTTCACTCAGCGTGATTATGATGTGCTGGCAGGACGATG | 4200 |
| QUERY: | 7467 | GACCTCCCCAGACTATACCATGTGGAAAAACGTGGGCAAGGAGCCGGCCCCCTTTAACCT | 7526 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 4201 | GACCTCCCCAGACTATACCATGTGGAAAAACGTGGGCAAGGAGCCGGCCCCCTTTAACCT | 4260 |
| QUERY: | 7527 | GTATATGTTCAAGAGCAACAATCCTCTCAGCAGTGAGCTAGATTTGAAGAACTACGTGAC | 7586 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 4261 | GTATATGTTCAAGAGCAACAATCCTCTCAGCAGTGAGCTAGATTTGAAGAACTACGTGAC | 4320 |
| QUERY: | 7587 | AGATGTGAAAAGCTGGCTTGTGATGTTTGGATTTCAGCTTAGCAACATCATTCCTGGCTT | 7646 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 4321 | AGATGTGAAAAGCTGGCTTGTGATGTTTGGATTTCAGCTTAGCAACATCATTCCTGGCTT | 4380 |
| QUERY: | 7647 | CCCGAGAGCCAAAATGTATTTCGTGCCTCCTCCCTATGAATTGTCAGAGAGTCAAGCAAG | 7706 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 4381 | CCCGAGAGCCAAAATGTATTTCGTGCCTCCTCCCTATGAATTGTCAGAGAGTCAAGCAAG | 4440 |
| QUERY: | 7707 | TGAGAATGGACAGCTCATTACAGGTGTCCAACAGACAACAGAGAGACATAACCAGGCCTT | 7766 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 4441 | TGAGAATGGACAGCTCATTACAGGTGTCCAACAGACAACAGAGAGACATAACCAGGCCTT | 4500 |
| QUERY: | 7767 | CATGGCTCTGGAAGGACAGGTCATTACTAAAAAGCTCCACGCCAGCATCCGAGAGAAAGC | 7826 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 4501 | CATGGCTCTGGAAGGACAGGTCATTACTAAAAAGCTCCACGCCAGCATCCGAGAGAAAGC | 4560 |
| QUERY: | 7827 | AGGTCACTGGTTTGCCACCACCACGCCCATCATTGGCAAAGGCATCATGTTTGCCATCAA | 7886 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 4561 | AGGTCACTGGTTTGCCACCACCACGCCCATCATTGGCAAAGGCATCATGTTTGCCATCAA | 4620 |
| QUERY: | 7887 | AGAAGGGCGGGTGACCACGGGCGTGTCCAGCATCGCCAGCGAAGATAGCCGCAAGGTGGC | 7946 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 4621 | AGAAGGGCGGGTGACCACGGGCGTGTCCAGCATCGCCAGCGAAGATAGCCGCAAGGTGGC | 4680 |
| QUERY: | 7947 | ATCTGTGCTGAACAACGCCTACTACCTGGACAAGATGCACTACAGCATCGAGGGCAAGGA | 8006 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 4681 | ATCTGTGCTGAACAACGCCTACTACCTGGACAAGATGCACTACAGCATCGAGGGCAAGGA | 4740 |
| QUERY: | 8007 | CACCCACTACTTTGTGAAGATTGGCTCAGCCGATGGCGACCTGGTCACACTAGGCACCAC | 8066 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 4741 | CACCCACTACTTTGTGAAGATTGGCTCAGCCGATGGCGACCTGGTCACACTAGGCACCAC | 4800 |
| QUERY: | 8067 | CATCGGCCGCAAGGTGCTAGAGAGCGGGGTGAACGTGACCGTGTCCCAGCCCACGCTGCT | 8126 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 4801 | CATCGGCCGCAAGGTGCTAGAGAGCGGGGTGAACGTGACCGTGTCCCAGCCCACGCTGCT | 4860 |
| QUERY: | 8127 | GGTCAACGGCAGGACTCGAAGGTTCACGAACATTGAGTTCCAGTACTCCACGCTGCTGCT | 8186 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 4861 | GGTCAACGGCAGGACTCGAAGGTTCACGAACATTGAGTTCCAGTACTCCACGCTGCTGCT | 4920 |
| QUERY: | 8187 | CAGCATCCGCTATGGCCTCACCCCCGACACCCTGGACGAAGAGAAGGCCCGCGTCCTGGA | 8246 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 4921 | CAGCATCCGCTATGGCCTCACCCCCGACACCCTGGACGAAGAGAAGGCCCGCGTCCTGGA | 4980 |
| QUERY: | 8247 | CCAGGCGAGACAGAGGGCCCTGGGCACGGCCTGGGCCAAGGAGCAGCAGAAAGCCAGGGA | 8306 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 4981 | CCAGGCGAGACAGAGGGCCCTGGGCACGGCCTGGGCCAAGGAGCAGCAGAAAGCCAGGGA | 5040 |
| QUERY: | 8307 | CGGGAGAGAGGGGAGCCGCCTGTGGACTGAGGGCGAGAAGCAGCAGCTTCTGAGCACCGG | 8366 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 5041 | CGGGAGAGAGGGGAGCCGCCTGTGGACTGAGGGCGAGAAGCAGCAGCTTCTGAGCACCGG | 5100 |
| QUERY: | 8367 | GCGCGTGCAAGGGTACGAGGGATATTACGTGCTTCCCGTGGAGCAATACCCAGAGCTTGC | 8426 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 5101 | GCGCGTGCAAGGGTACGAGGGATATTACGTGCTTCCCGTGGAGCAATACCCAGAGCTTGC | 5160 |
| QUERY: | 8427 | AGACAGTAGCAGCAACATCCAGTTTTTAAGACAGAATGAGATGGGAAAGAGGTAACAAAA | 8486 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 5161 | AGACAGTAGCAGCAACATCCAGTTTTTAAGACAGAATGAGATGGGAAAGAGGTAACAAAA | 5220 |
| QUERY: | 8487 | TAATCTGCTGCCATTCCTTGTCTGAATGGCTCAGCAGGAGTAACTGTTATCTCCTCTCCT | 8546 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 5221 | TAATCTGCTGCCATTCCTTGTCTGAATGGCTCAGCAGGAGTAACTGTTATCTCCTCTCCT | 5280 |
| QUERY: | 8547 | AAGGAGATGAAGACCTAACAGGGGCACTGCGGCTGGGCTGCTTTAGGAGACCAAGTGGCA | 8606 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 5281 | AAGGAGATGAAGACCTAACAGGGGCACTGCGGCTGGGCTGCTTTAGGAGACCAAGTGGCA | 5340 |
| QUERY: | 8607 | AGAAAGCTCACATTTTTTGAGTTCAAATGCTACTGTCCAAGCGAGAAGTCCCTCATCCTG | 8666 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 5341 | AGAAAGCTCACATTTTTTGAGTTCAAATGCTACTGTCCAAGCGAGAAGTCCCTCATCCTG | 5400 |
| QUERY: | 8667 | AAGTAGACTAAAGCCCGGCTGAAAATTCCGAGGAAAACAAAACAAACGAATGAATGAACA | 8726 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 5401 | AAGTAGACTAAAGCCCGGCTGAAAATTCCGAGGAAAACAAAACAAACGAATGAATGAACA | 5460 |
| QUERY: | 8727 | GACACACACAATGTTCCAAGTTCCCCTAAAATATGACCCACTTGTTCTGGGTCTACGCAG | 8786 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 5461 | GACACACACAATGTTCCAAGTTCCCCTAAAATATGACCCACTTGTTCTGGGTCTACGCAG | 5520 |
| QUERY: | 8787 | AAAAGAGACGCAAAGTGT 8804 | |
| | | \|\|\|\|\|\|\|\|\|\|\|\|\|\|\|\|\|\| | |
| SBJCT: | 5521 | AAAAGAGACGCAAAGTGT 5538 | |

SCORE = 1362 BITS (687), EXPECT = 0.0
IDENTITIES = 705/714 (98%)
STRAND = PLUS/PLUS

| | | | |
|---|---|---|---|
| QUERY: | 8875 | CACGGACCGATAAACAAAGAAGCGAAGATAAGAAAGAAGGCCTCATATCCAATTACCTCA | 8934 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 5609 | CACGGACCGATAAACAAAGAAGCGAAGATAAGAAAGAAGGCCTCATATCCAATTACCTCA | 5668 |
| QUERY: | 8935 | CTCATTCACATGTGAGCGACACGCAGACATCCGCGAGGGCCAGCGTCACCAGACCAGCTG | 8994 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 5669 | CTCATTCACATGTGAGCGACACGCAGACATCCGCGAGGGCCAGCGTCACCAGACCAGCTG | 5728 |
| QUERY: | 8995 | CGGGACAAACCACTCAGACTGCTTGTAGGACAAATACTTCTGACATTTTCGTTTAAGCAA | 9054 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 5729 | CGGGACAAACCACTCAGACTGCTTGTAGGACAAATACTTCTGACATTTTCGTTTAAGCAA | 5788 |
| QUERY: | 9055 | ATACAGGTGCATTTAAAACACGACTTTGGGGGTGATTTGTGTGTAGCGCCTGGGGAGGGG | 9114 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 5789 | ATACAGGTGCATTTAAAACACGACTTTGGGGGTGATTTGTGTGTAGCGCCTGGGGAGGGG | 5848 |
| QUERY: | 9115 | GGATAAAAGAGGAGGAGTGAGCACTGGAAATACTTTTTAAAGNNNNNNNNNCATGAGGGA | 9174 |
| | | \|\|\|\|\|\|\|\|\|\|\|\|\|\|\|\|\|\|\|\|\|\|\|\|\|\|\|\|\|\|\|\|\|\|\|\|\|\|\|\|\|\| \|\|\|\|\|\|\|\|\| | |
| SBJCT: | 5849 | GGATAAAAGAGGAGGAGTGAGCACTGGAAATACTTTTTAAAGAAAAAAAAACATGAGGGA | 5908 |
| QUERY: | 9175 | ATAAAAGAAATTCCTATCAAAAATCAAAGTGAAATAATACCATCCAGCACTTAACTCTCA | 9234 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 5909 | ATAAAAGAAATTCCTATCAAAAATCAAAGTGAAATAATACCATCCAGCACTTAACTCTCA | 5968 |
| QUERY: | 9235 | GGTCCCAACTAAGTCTGGCCTGAGCTAATTTATTTGAGCGCAGAGTGTAAAATTTAATTC | 9294 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 5969 | GGTCCCAACTAAGTCTGGCCTGAGCTAATTTATTTGAGCGCAGAGTGTAAAATTTAATTC | 6028 |
| QUERY: | 9295 | AAAATGGTGGCTATAATCACTACAGATAAATTTCATACTCTTTTGTCTTTGGAGATTCCA | 9354 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 6029 | AAAATGGTGGCTATAATCACTACAGATAAATTTCATACTCTTTTGTCTTTGGAGATTCCA | 6088 |
| QUERY: | 9355 | TTGTGGACAGTAATACGCAGTTACAGGGTGTAGTCTGTTTAGATTCCGTAGTTCGTGGGT | 9414 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 6089 | TTGTGGACAGTAATACGCAGTTACAGGGTGTAGTCTGTTTAGATTCCGTAGTTCGTGGGT | 6148 |
| QUERY: | 9415 | ATCAGTTTCGGTAGAGGTGCAGCATCGTGACACTTTTGCTAACAGGTACCACTTCTGATC | 9474 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 6149 | ATCAGTTTCGGTAGAGGTGCAGCATCGTGACACTTTTGCTAACAGGTACCACTTCTGATC | 6208 |
| QUERY: | 9475 | ACCCTGTACATACATGAGCCGAAAGGCACAATCACTGTTTCAGATTTAAAATTATTAGTG | 9534 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 6209 | ACCCTGTACATACATGAGCCGAAAGGCACAATCACTGTTTCAGATTTAAAATTATTAGTG | 6268 |
| QUERY: | 9535 | TGTTTGTTTGGTCCAGAAACTGAGACAATCACATGACAGTCACCACGAGGAGAG 9588 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 6269 | TGTTTGTTTGGTCCAGAAACTGAGACAATCACATGACAGTCACCACGAGGAGAG 6322 | |

SCORE = 349 BITS (176), EXPECT = 2E-92
IDENTITIES = 176/176 (100%)
STRAND = PLUS/PLUS

| | | | |
|---|---|---|---|
| QUERY: | 9651 | GTCTAATAAGAACTTTGGTACAGGAACTTTTTTGTAATATACATGTATGAATTGTTCATC | 9710 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 6385 | GTCTAATAAGAACTTTGGTACAGGAACTTTTTTGTAATATACATGTATGAATTGTTCATC | 6444 |
| QUERY: | 9711 | GAGTTTTTATATTAATTTTAATTTGCTGCTAAGCAAAGACTAGGGACAGGCAAAGATAAT | 9770 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 6445 | GAGTTTTTATATTAATTTTAATTTGCTGCTAAGCAAAGACTAGGGACAGGCAAAGATAAT | 6504 |
| QUERY: | 9771 | TTATGGCAAAGTGTTTAAATTGTTTATACATAAATAAAGTCTCTAAAACTCCTGTG 9826 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 6505 | TTATGGCAAAGTGTTTAAATTGTTTATACATAAATAAAGTCTCTAAAACTCCTGTG 6560 | |

In this search it was also found that the FCTR3bcd and e nucleic acids had homology to five fragments of Mus musculus mRNA for Ten-m2. It has 5498 of 6108 bases (90%) identical to bases 2504-8610, 1095 of 1196 bases (91%) identical to bases 103-1298, 1000 of 1088 bases (91%) identical to bases 1420-2540, 81 of 89 bases (91%) identical to bases 8655-8743, and 30 of 32 bases (93%) identical to bases 7-38 of Mus musculus mRNA for Ten-m2 (Table 3M).

TABLE 3M

BLASTN of FCTR3b, c, d, and e against Mus musculus mRNA for Ten-m2 (SEQ ID
NO:65) Mrna

>GI|4760777|DBJ|AB025411.1|AB025411 MUS MUSCULUS MRNA FOR TEN-M2, COMPLETE
CDS
LENGTH = 8797
SCORE = 7263 BITS (3664), EXPECT = 0.0
IDENTITIES = 5498/6108 (90%), GAPS = 1/6108 (0%)
STRAND = PLUS/PLUS

| | | | |
|---|---|---|---|
| QUERY: | 2578 | GATGGCTGCCCTGACTTGTGCAACGGTAACGGGAGATGCACACTGGGTCAGAACAGCTGG | 2637 |
| | | \|\|\|\|\|\|\|\|\|\|\|\|\|\| \|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 2504 | GATGGCTGCCCTGATTTGTGCAACGGTAACGGGAGATGCACACTGGGTCAGAACAGCTGG | 2563 |
| QUERY: | 2638 | CAGTGTGTCTGCCAGACCGGCTGGAGAGGGCCCGGATGCAACGTTGCCATGGAAACTTCC | 2697 |
| | | \|\|\|\|\|\|\|\|\|\|\|\|\|\|\|\|\|\|\|\|\|\|\|\|\|\|\|\|\|\|\|\| \|\|\|\|\|\|\|\|\|\|\|\|\|\|\|\|\|\|\|\|\|\|\| \|\|\| | |
| SBJCT: | 2564 | CAGTGTGTCTGCCAGACCGGCTGGAGAGGGCCTGGATGCAACGTTGCCATGGAAACCTCC | 2623 |
| QUERY: | 2698 | TGTGCTGATAACAAGGATAATGAGGGAGATGGCCTGGTGGATTGTTTGGACCCTGACTGC | 2757 |
| | | \|\| \|\|\|\|\|\|\|\|\|\|\|\|\|\|\|\|\|\|\|\|\|\|\|\|\|\|\|\|\|\|\|\|\|\|\|\|\|\| \|\| \|\|\|\|\|\|\|\|\|\|\|\|\|\| | |
| SBJCT: | 2624 | TGCGCTGATAACAAGGATAATGAGGGAGATGGCCTGGTGGACTGCCTGGACCCTGACTGC | 2683 |
| QUERY: | 2758 | TGCCTGCAGTCAGCCTGTCAGAACAGCCTGCTCTGCCGGGGGTCCCGGGACCCACTGGAC | 2817 |
| | | \|\|\|\|\| \|\|\|\|\|\|\|\|\|\|\|\|\|\|\|\|\|\|\|\|\|\|\|\|\|\|\|\|\|\|\|\|\|\|\|\|\|\| \|\|\|\|\|\|\|\| \|\|\|\|\| | |
| SBJCT: | 2684 | TGCCTACAGTCAGCCTGTCAGAACAGCCTGCTCTGCCGGGGGTCTCGGGACCCCTTGGAC | 2743 |
| QUERY: | 2818 | ATCATTCAGCAGGGCCAGACGGATTGGCCCGCAGTGAAGTCCTTCTATGACCGTATCAAG | 2877 |
| | | \|\|\|\|\|\|\|\|\|\|\| \|\| \|\|\|\|\| \|\| \|\|\|\|\| \|\|\|\|\|\|\|\|\|\|\|\|\|\|\|\|\|\|\|\|\|\|\| \|\|\|\|\|\| | |
| SBJCT: | 2744 | ATCATTCAGCAAGGTCAGACAGACTGGCCTGCAGTGAAGTCCTTCTATGACCGCATCAAG | 2803 |
| QUERY: | 2878 | CTCTTGGCAGGCAAGGATAGCACCCACATCATTCCTGGAGAGAACCCTTTCAACAGCAGC | 2937 |
| | | \|\|\|\|\|\|\|\|\|\|\|\|\|\|\|\|\| \|\|\|\|\|\|\|\|\|\|\|\|\|\|\|\|\|\|\|\|\|\|\| \|\|\|\|\| \|\|\|\|\| \|\|\|\|\|\| | |
| SBJCT: | 2804 | CTCTTGGCAGGCAAGGACAGCACCCACATCATTCCTGGAGACAACCCCTTCAATAGCAGC | 2863 |
| QUERY: | 2938 | TTGGTTTCTCTCATCCGAGGCCAAGTAGTAACTACAGATGGAACTCCCCTGGTCGGTGTG | 2997 |
| | | \|\|\|\| \|\|\|\|\| \|\|\|\|\|\|\|\|\|\|\|\|\|\|\|\|\|\|\|\| \| \|\|\|\|\| \|\|\|\|\|\| \|\|\|\| \|\|\|\|\|\| | |
| SBJCT: | 2864 | CTGGTGTCTCTGATCCGAGGCCAAGTAGTAACCATGGATGGGACTCCCTTGGTGGGTGTG | 2923 |
| QUERY: | 2998 | AACGTGTCTTTTGTCAAGTACCCAAAATACGGCTACACCATCACCCGCCAGGATGGCACG | 3057 |
| | | \|\| \|\|\|\|\|\|\|\|\|\|\|\|\|\|\|\|\|\|\|\|\|\|\|\|\|\| \|\|\|\|\|\|\|\|\|\|\|\|\|\| \|\|\|\|\|\|\|\|\|\|\|\|\|\|\| | |
| SBJCT: | 2924 | AATGTGTCTTTTGTCAAGTACCCAAAATATGGCTACACCATCACTCGCCAGGATGGCACG | 2983 |
| QUERY: | 3058 | TTCGACCTGATCGCAAATGGAGGTGCTTCCTTGACTCTACACTTTGAGCGAGCCCCGTTC | 3117 |
| | | \|\| \|\|\|\|\|\|\|\| \|\| \|\|\|\|\| \|\|\| \|\| \|\|\|\|\|\|\|\|\|\| \|\|\|\|\|\|\|\|\|\|\|\|\|\|\|\|\| \|\|\| | |
| SBJCT: | 2984 | TTTGACCTGATTGCCAATGGGGGTTCTGCCTTGACTCTTCACTTTGAGCGAGCCCCTTTC | 3043 |
| QUERY: | 3118 | ATGAGCCAGGAGCGCACTGTGTGGCTGCCGTGGAACAGCTTTTACGCCATGGACACCCTG | 3177 |
| | | \|\|\|\|\|\|\|\|\|\|\|\|\|\|\|\|\| \|\|\|\|\|\|\|\|\|\|\| \|\|\|\|\|\|\|\|\|\|\| \|\| \|\|\|\|\|\|\|\|\|\|\|\|\|\|\| | |
| SBJCT: | 3044 | ATGAGCCAGGAGCGCACAGTGTGGCTGCCATGGAACAGCTTCTATGCCATGGACACCCTG | 3103 |
| QUERY: | 3178 | GTGATGAAGACCGAGGAGAACTCCATCCCCAGCTGTGACCTCAGTGGCTTTGTCCGGCCT | 3237 |
| | | \|\| \|\|\|\|\|\|\|\|\|\|\|\|\|\| \|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 3104 | GTAATGAAGACCGAGGAAAACTCCATCCCCAGCTGTGACCTCAGTGGCTTTGTCCGGCCA | 3163 |
| QUERY: | 3238 | GATCCAATCATCATCTCCTCCCCACTGTCCACCTTCTTTAGTGCTGCCCCTGGGCAGAAT | 3297 |
| | | \|\|\|\|\|\|\|\|\|\|\|\|\|\|\|\|\|\|\|\| \|\| \|\|\|\|\|\|\|\|\|\|\|\|\|\| \|\| \|\|\| \|\|\|\|\|\| \|\|\| | |
| SBJCT: | 3164 | GATCCAATCATCATCTCCTCTCCTCTGTCCACCTTCTTCAGCGCTTCCCCTGCCTCGAAC | 3223 |
| QUERY: | 3298 | CCCATCGTGCCTGAGACCCAGGTTCTTCATGAAGAAATCGAGCTCCCTGGTTCCAATGTG | 3357 |
| | | \|\|\|\|\| \|\|\|\|\|\|\|\|\|\|\|\|\|\|\|\|\|\|\|\|\|\|\|\|\|\|\|\|\|\|\|\| \|\|\|\|\|\|\|\|\|\|\|\| \|\|\|\|\|\|\|\| | |
| SBJCT: | 3224 | CCCATTGTGCCTGAGACCCAGGTTCTTCATGAAGAAATTGAGCTCCCTGGTACCAATGTG | 3283 |
| QUERY: | 3358 | AAACTTCGCTATCTGAGCTCTAGAACTGCAGGGTACAAGTCACTGCTGAAGATCACCATG | 3417 |
| | | \|\| \|\| \|\| \|\|\|\|\| \|\|\|\|\|\|\|\|\|\|\|\|\|\|\|\|\|\|\|\| \|\|\|\|\| \|\|\|\|\|\|\|\|\|\|\|\|\|\|\|\|\|\| | |
| SBJCT: | 3284 | AAGCTCCGTTATCTCAGCTCTAGAACTGCAGGGTATAAGTCGCTGCTGAAGATCACCATG | 3343 |
| QUERY: | 3418 | ACCCAGTCCACAGTGCCCCTGAACCTCATTAGGGTTCACCTGATGGTGGCTGTCGAGGGG | 3477 |
| | | \|\| \|\|\|\|\|\|\|\|\|\|\|\|\|\|\| \|\|\|\|\|\|\|\|\|\| \|\|\|\|\|\|\|\|\| \|\|\|\|\|\|\|\| \|\|\|\|\| \|\|\|\|\| | |
| SBJCT: | 3344 | ACGCAGTCCACAGTGCCCTTGAACCTCATCAGGGTTCACTTGATGGTTGCTGTAGAGGGG | 3403 |
| QUERY: | 3478 | CATCTCTTCCAGAAGTCATTCCAGGCTTCTCCCAACCTGGCCTCCACCTTCATCTGGGAC | 3537 |
| | | \|\|\|\|\|\|\|\|\|\|\|\|\|\|\|\|\|\|\|\|\|\|\|\|\|\|\|\|\|\|\|\|\|\|\|\|\|\| \|\|\|\| \|\|\| \|\|\|\|\|\|\|\|\|\|\|\| | |
| SBJCT: | 3404 | CATCTCTTCCAGAAGTCATTCCAGGCTTCTCCCAACCTAGCCTACACATTCATCTGGGAC | 3463 |
| QUERY: | 3538 | AAGACAGATGCGTATGGCCAAAGGGTGTATGGACTCTCAGATGCTGTTGTGTCTGTCGGG | 3597 |
| | | \|\|\|\|\|\|\|\|\|\|\| \|\|\|\|\|\|\|\|\|\|\|\|\|\| \|\|\|\|\| \|\| \|\| \|\|\|\|\|\|\|\|\|\|\|\|\|\|\|\|\| \|\|\| | |
| SBJCT: | 3464 | AAGACAGATGCTTATGGCCAAAGGGTTTATGGCCTATCGGATGCTGTTGTGTCTGTTGGG | 3523 |
| QUERY: | 3598 | TTTGAATATGAGACCTGTCCCAGTCTAATTCTCTGGGAGAAAAGGACAGCCCTCCTTCAG | 3657 |
| | | \|\|\|\|\|\|\|\|\|\|\|\|\|\|\|\|\| \|\|\|\|\|\|\|\| \|\| \|\| \|\|\|\|\|\|\|\|\|\|\|\|\|\|\|\|\|\|\|\| \|\|\|\|\|\| | |
| SBJCT: | 3524 | TTTGAATATGAGACCTGCCCCAGTCTCATCCTGTGGGAGAAAAGGACAGCCCTGCTTCAG | 3583 |
| QUERY: | 3658 | GGATTCGAGCTGGACCCCTCCAACCTCGGTGGCTGGTCCCTAGACAAACACCACATCCTC | 3717 |
| | | \|\|\|\|\|\|\|\|\|\|\|\|\|\|\|\|\| \|\|\|\|\|\|\|\| \|\| \|\|\|\|\|\|\|\|\|\|\| \|\|\|\|\|\|\|\|\|\|\|\|\| \|\|\|\| | |
| SBJCT: | 3584 | GGATTCGAGCTGGACCCTTCCAACCTTGGAGGCTGGTCCCTGGACAAACACCACACCCTC | 3643 |
| QUERY: | 3718 | AATGTTAAAAGTGGAATCCTACACAAAGGCACTGGGGAAAACCAGTTCCTGACCCAGCAG | 3777 |
| | | \|\|\|\|\| \|\|\|\|\| \|\|\|\|\| \|\|\|\|\|\|\|\|\|\|\| \|\| \|\|\|\|\| \|\|\|\|\|\|\|\|\|\|\|\|\|\|\|\|\|\|\|\|\| | |
| SBJCT: | 3644 | AATGTGAAAAGCGGAATACTACACAAAGGGACAGGGGAGAACCAGTTCCTGACCCAGCAG | 3703 |
| QUERY: | 3778 | CCTGCCATCATCACCAGCATCATGGGCAATGGTCGCCGCCGGAGCATTTCCTGTCCCAGC | 3837 |
| | | \|\|\|\|\|\|\|\|\|\|\|\|\|\| \|\|\|\|\|\|\|\|\|\|\|\|\|\| \|\|\|\|\|\|\|\|\| \| \|\|\|\|\| \|\|\|\|\|\|\|\|\|\|\|\| | |
| SBJCT: | 3704 | CCTGCCATCATCACGAGCATCATGGGCAACGGTCGCCGCAGAAGCATCTCCTGTCCCAGC | 3763 |
| QUERY: | 3838 | TGCAACGGCCTTGCTGAAGGCAACAAGCTGCTGGCCCCAGTGGCTCTGGCTGTTGGAATC | 3897 |
| | | \|\|\|\|\| \|\|\|\|\|\|\|\|\|\|\|\|\|\|\|\|\|\|\|\| \|\|\| \| \|\|\|\|\| \|\|\|\|\| \|\|\|\|\|\|\|\| \|\| \|\|\| | |
| SBJCT: | 3764 | TGCAATGGCCTTGCTGAAGGCAACAAACTGTTAGCCCCTGTGGCCCTGGCTGTGGGGATC | 3823 |
| QUERY: | 3898 | GATGGGAGCCTCTATGTGGGTGACTTCAATTACATCCGACGCATCTTTCCCTCTCGAAAT | 3957 |
| | | \|\|\|\|\|\|\|\|\|\|\|\|\| \|\|\| \|\|\|\|\|\|\|\|\|\|\| \|\| \|\|\|\| \|\|\|\|\|\|\|\|\|\|\|\|\|\|\|\|\|\|\|\|\|\| | |
| SBJCT: | 3824 | GATGGGAGCCTCTTTGTTGGTGACTTCAACTATATCCGGCGCATCTTTCCCTCTCGAAAT | 3883 |
| QUERY: | 3958 | GTGACCAGCATCTTGGAGTTACGAAATAAAGAGTTTAAACATAGCAACAACCCAGCACAC | 4017 |
| | | \|\|\|\|\|\|\|\| \|\|\|\|\|\|\|\|\|\|\|\|\|\|\|\|\|\|\|\|\|\|\|\|\|\|\|\|\|\|\|\|\|\|\|\|\|\|\|\| \|\|\|\|\| \|\|\|\| | |
| SBJCT: | 3884 | GTGACCAGTATCTTGGAGTTACGAAATAAAGAGTTTAAACATAGCAACAGCCCAGGACAC | 3943 |
| QUERY: | 4018 | AAGTACTACTTGGCAGTGGACCCCGTGTCCGGCTCGCTCTACGTGTCCGACACCAACAGC | 4077 |
| | | \|\|\|\|\|\|\|\|\|\|\|\|\|\| \|\|\|\|\|\|\|\|\|\|\|\| \| \|\|\|\|\| \|\|\|\|\|\|\|\| \|\| \|\|\|\|\|\|\|\|\|\|\|\| | |
| SBJCT: | 3944 | AAGTACTACTTGGCTGTGGACCCCGTGACTGGCTCACTCTACGTCTCTGACACCAACAGT | 4003 |
| QUERY: | 4078 | AGGAGAATCTACCGCGTCAAGTCTCTGAGTGGAACCAAAGACCTGGCTGGGAATTCGGAA | 4137 |
| | | \| \|\|\|\|\|\|\|\|\|\|\| \|\|\|\|\|\|\|\|\|\|\|\|\|\| \|\|\| \|\|\|\|\|\|\|\|\|\|\|\|\|\|\|\| \|\|\|\|\|\|\|\|\| | |
| SBJCT: | 4004 | CGCCGAATCTACCGAGTCAAGTCTCTGAGCGGAGCCAAAGACCTGGCTGGAAATTCGGAA | 4063 |
| QUERY: | 4138 | GTTGTGGCAGGGACGGGAGAGCAGTGTCTACCCTTTGATGAAGCCCGCTGCGGGGATGGA | 4197 |
| | | \|\|\|\|\|\|\|\|\|\|\|\|\|\| \|\| \|\| \|\| \|\|\|\|\|\|\|\|\|\|\|\|\|\|\|\|\|\|\|\|\|\|\|\|\|\| \|\|\|\|\|\|\|\|\| | |
| SBJCT: | 4064 | GTTGTGGCAGGGACTGGCGAACAATGTCTACCCTTTGATGAAGCCCGCTGTGGGGATGGA | 4123 |
| QUERY: | 4198 | GGGAAGGCCATAGATGCAACCCTGATGAGCCCGAGAGGTATTGCAGTAGACAAGAATGGG | 4257 |
| | | \|\|\|\|\|\|\|\| \| \|\| \|\| \|\|\|\|\|\|\|\|\|\|\|\|\|\| \|\|\|\|\|\|\|\|\|\|\|\|\|\|\|\|\|\|\|\|\|\|\|\|\|\|\| | |
| SBJCT: | 4124 | GGGAAGGCTGTGGACGCCACCCTGATGAGCCCCAGAGGTATTGCAGTAGACAAGAATGGG | 4183 |
| QUERY: | 4258 | CTCATGTACTTTGTCGATGCCACCATGATCCGGAAGGTTGACCAGAATGGAATCATCTCC | 4317 |
| | | \|\| \|\|\|\|\|\|\|\|\|\|\| \|\|\|\|\|\|\|\|\|\|\|\|\|\|\|\|\|\|\|\|\|\|\| \|\|\|\|\| \|\| \|\|\|\|\|\|\|\|\|\|\|\| | |
| SBJCT: | 4184 | CTTATGTACTTTGTTGATGCCACCATGATCCGGAAGGTGGACCAAAACGGAATCATCTCC | 4243 |
| QUERY: | 4318 | ACCCTGCTGGGCTCCAATGACCTCACTGCCGTCCGGCCGCTGAGCTGTGATTCCAGCATG | 4377 |
| | | \|\|\|\|\|\|\|\|\|\|\|\|\|\|\|\|\|\|\|\|\|\|\|\|\|\| \|\| \|\|\|\|\| \|\| \|\|\|\|\|\|\|\|\|\|\| \|\| \|\|\|\|\|\| | |
| SBJCT: | 4244 | ACCCTGCTGGGCTCCAATGACCTCACAGCTGTCCGACCACTGAGCTGTGACTCGAGCATG | 4303 |
| QUERY: | 4378 | GATGTAGCCCAGGTTCGTCTGGAGTGGCCAACAGACCTTGCTGTCAATCCCATGGATAAC | 4437 |
| | | \|\| \|\| \|\|\|\|\|\|\|\| \|\|\|\|\| \|\| \|\|\|\|\| \|\|\|\|\|\|\|\| \|\| \|\|\|\|\| \|\|\|\|\|\|\|\| \|\|\| | |
| SBJCT: | 4304 | GACGTGGCCCAGGTCCGTCTAGAATGGCCGACAGACCTCGCCGTCAACCCCATGGACAAC | 4363 |
| QUERY: | 4438 | TCCTTGTATGTTCTAGAGAACAATGTCATCCTTCGAATCACCGAGAACCACCAAGTCAGC | 4497 |
| | | \|\|\| \|\|\|\| \|\|\|\|\| \|\|\|\|\|\|\|\| \|\|\|\|\|\|\|\| \|\| \|\|\|\|\| \|\|\|\|\|\|\|\|\|\|\| \|\|\|\|\|\| | |
| SBJCT: | 4364 | TCCCTGTACGTTCTGGAGAACAACGTCATCCTGCGGATCACGGAGAACCACCAGGTCAGC | 4423 |
| QUERY: | 4498 | ATCATTGCGGGACGCCCCATGCACTGCCAAGTTCCTGGCATTGACTACTCACTCAGCAAA | 4557 |
| | | \|\|\|\|\| \|\|\|\|\|\|\|\| \|\| \|\|\|\|\|\|\|\|\|\|\| \|\|\|\|\| \|\|\|\|\| \|\|\|\|\|\|\|\| \|\|\|\|\|\|\|\|\| | |
| SBJCT: | 4424 | ATCATCGCGGGACGGCCTATGCACTGCCAGGTTCCCGGCATCGACTACTCGCTCAGCAAA | 4483 |
| QUERY: | 4558 | CTAGCCATTCACTCTGCCCTGGAGTCAGCCAGTGCCATTGCCATTTCTCACACTGGGGTC | 4617 |
| | | \|\| \|\|\|\|\| \|\|\|\|\|\|\|\| \|\|\|\|\| \|\|\|\|\|\|\|\| \|\|\|\|\|\|\|\|\|\|\|\|\|\|\|\|\|\|\|\|\|\|\|\|\|\|\| | |
| SBJCT: | 4484 | CTCGCCATCCACTCTGCGCTGGAATCAGCCAGCGCCATTGCCATTTCTCACACTGGGGTG | 4543 |
| QUERY: | 4618 | CTCTACATCACTGAGACAGATGAGAAGAAGATTAACCGTCTACGCCAGGTAACAACCAAC | 4677 |
| | | \|\|\|\|\|\|\|\|\|\|\|\|\|\|\|\|\| \|\| \|\|\|\|\|\|\|\|\|\|\| \|\|\|\|\| \|\|\|\|\|\|\|\| \|\| \|\| \|\|\|\|\| | |
| SBJCT: | 4544 | CTCTACATCACTGAGACGGACGAGAAGAAGATCAACCGCCTACGCCAAGTCACCACCAAT | 4603 |
| QUERY: | 4678 | GGGGAGATCTGCCTTTTAGCTGGGGCAGCCTCGGACTGCGACTGCAAAAACGATGTCAAT | 4737 |
| | | \|\| \|\|\|\|\|\|\|\|\|\|\| \|\|\|\|\| \|\|\|\|\| \|\|\|\|\| \|\|\|\|\| \|\|\|\|\|\|\|\|\|\|\|\|\|\|\|\|\|\|\|\| | |
| SBJCT: | 4604 | GGAGAGATCTGCCTCTTAGCCGGGGCGGCCTCAGACTGTGACTGCAAAAACGATGTCAAC | 4663 |
| QUERY: | 4738 | TGCAACTGCTATTCAGGAGATGATGCCTACGCGACTGATGCCATCTTGAATTCCCCATCA | 4797 |
| | | \|\|\|\| \|\|\|\|\|\| \|\| \|\|\|\|\|\|\|\| \|\| \|\|\|\|\| \|\| \|\| \|\|\|\|\|\| \|\|\|\| \|\| \|\| \|\| | |
| SBJCT: | 4664 | TGCATCTGCTACTCGGGAGATGACGCTTACGCCACGGACGCCATCCTGAACTCGCCGTCC | 4723 |
| QUERY: | 4798 | TCCTTAGCTGTAGCTCCAGATGGTACCATTTACATTGCAGACCTTGGAAATATTCGGATC | 4857 |
| | | \|\|\|\|\|\|\|\| \|\| \|\|\|\|\| \|\|\|\|\| \|\|\|\|\| \|\|\|\|\|\|\|\|\|\|\|\|\|\|\|\|\| \|\|\|\|\| \|\|\|\|\|\| | |
| SBJCT: | 4724 | TCCTTAGCCGTGGCTCCGGATGGCACCATCTACATTGCAGACCTTGGGAATATCCGGATC | 4783 |
| QUERY: | 4858 | AGGGCGGTCAGCAAGAACAAGCCTGTTCTTAATGCCTTCAACCAGTATGAGGCTGCATCC | 4917 |
| | | \|\|\|\|\|\|\|\|\|\|\|\|\|\| \|\| \|\| \|\| \|\|\|\|\|\|\|\| \|\| \|\|\|\|\|\|\|\|\|\|\|\|\|\|\|\|\|\|\|\|\|\|\| | |
| SBJCT: | 4784 | AGGGCGGTCAGCAAAAATAAACCCGTTCTTAACGCATTCAACCAGTATGAGGCTGCATCT | 4843 |
| QUERY: | 4918 | CCCGGAGAGCAGGAGTTATATGTTTTCAACGCTGATGGCATCCACCAATACACTGTGAGC | 4977 |
| | | \|\| \|\|\|\|\| \|\|\|\|\| \|\| \|\| \|\| \|\|\|\|\|\|\|\|\|\|\|\|\|\| \|\|\|\|\| \|\| \|\|\|\|\|\|\|\|\|\|\| | |
| SBJCT: | 4844 | CCGGGAGAACAGGAATTGTACGTGTTCAACGCTGATGGTATCCATCAGTACACTGTGAGT | 4903 |
| QUERY: | 4978 | CTGGTGACAGGGGAGTACTTGTACAATTTCACATATAGTACTGACAATGATGTCACTGAA | 5037 |
| | | \|\|\|\|\|\|\|\| \|\|\|\|\|\|\|\|\|\|\|\|\|\|\|\|\|\|\|\|\|\|\|\|\|\| \|\| \|\|\|\|\|\|\|\|\|\|\| \|\|\|\|\| \|\| | |
| SBJCT: | 4904 | CTGGTGACTGGGGAGTACTTGTACAATTTCACATACAGCGCTGACAATGACGTCACCGAG | 4963 |
| QUERY: | 5038 | TTGATTGACAATAATGGGAATTCCCTGAAGATCCGTCGGGACAGCAGTGGCATGCCCCGT | 5097 |
| | | \|\|\|\|\|\|\|\|\|\|\| \|\| \|\|\|\|\|\|\|\|\|\|\| \|\|\|\|\|\|\|\| \|\|\|\|\|\|\|\|\|\|\|\|\|\|\|\|\|\|\|\|\|\| | |
| SBJCT: | 4964 | TTGATTGACAACAACGGGAATTCCCTAAAGATCCGCCGGGACAGCAGTGGCATGCCCCGC | 5023 |
| QUERY: | 5098 | CACCTGCTCATGCCTGACAACCAGATCATCACCCTCACCGTGGGCACCAATGGAGGCCTC | 5157 |
| | | \|\|\|\|\|\|\|\|\|\|\|\|\|\| \|\| \|\| \|\|\|\|\| \|\|\|\|\|\|\|\| \|\| \|\|\|\|\|\|\|\|\|\|\|\|\|\|\|\|\|\|\|\|\| | |
| SBJCT: | 5024 | CACCTGCTCATGCCGGATAATCAGATTATCACCCTTACTGTGGGCACCAATGGAGGCCTC | 5083 |
| QUERY: | 5158 | AAAGTCGTGTCCACACAGAACCTGGAGCTTGGTCTCATGACCTATGATGGCAACACTGGG | 5217 |
| | | \|\|\|\| \|\|\|\|\|\|\|\|\| \|\|\|\|\|\|\|\|\|\|\|\|\|\| \|\| \|\|\|\|\|\|\|\| \|\|\|\|\|\|\|\| \|\|\|\|\|\|\|\| | |
| SBJCT: | 5084 | AAAGCCGTGTCCACTCAGAACCTGGAGCTGGGCCTCATGACTTATGATGGGAACACTGGA | 5143 |
| QUERY: | 5218 | CTCCTGGCCACCAAGAGCGATGAAACAGGATGGACGACTTTCTATGACTATGACCACGAA | 5277 |
| | | \|\|\|\|\| \|\|\|\|\|\|\|\|\|\|\| \|\|\|\|\|\|\|\| \|\|\|\|\|\|\|\| \|\|\|\|\| \|\|\|\|\|\|\|\|\|\|\|\|\|\|\|\|\| | |
| SBJCT: | 5144 | CTCCTAGCCACCAAGAGTGATGAAACCGGATGGACAACTTTTTATGACTATGACCACGAG | 5203 |
| QUERY: | 5278 | GGCCGCCTGACCAACGTGACGCGCCCCACGGGGGTGGTAACCAGTCTGCACCGGGAAATG | 5337 |
| | | \|\|\|\|\| \|\|\|\|\|\|\|\| \|\|\|\|\| \|\|\|\|\|\|\|\|\|\|\| \|\|\|\|\| \|\|\|\|\|\|\|\|\|\|\|\|\|\|\|\|\|\|\|\|\| | |
| SBJCT: | 5204 | GGCCGTCTGACCAATGTGACCCGCCCCACGGGCGTGGTGACCAGTCTGCACCGGGAAATG | 5263 |
| QUERY: | 5338 | GAGAAATCTATTACCATTGACATTGAGAACTCCAACCGTGATGATGACGTCACTGTCATC | 5397 |
| | | \|\|\|\|\|\|\|\|\|\|\| \|\|\|\|\|\|\|\|\|\|\|\|\|\|\|\|\|\|\|\|\|\|\|\|\|\| \|\|\|\|\|\|\|\|\|\|\|\|\|\|\|\|\| \|\|\| | |
| SBJCT: | 5264 | GAGAAATCTATCACCATTGACATTGAGAACTCCAACCGGGATGATGACGTCACTGTGATC | 5323 |
| QUERY: | 5398 | ACCAACCTCTCTTCAGTAGAGGCCTCCTACACAGTGGTACAAGATCAAGTTCGGAACAGC | 5457 |
| | | \|\|\|\|\|\|\|\|\|\|\| \|\| \|\| \|\|\|\|\|\|\|\|\|\|\| \|\|\|\|\|\|\|\|\|\|\|\|\|\|\|\|\|\|\|\| \|\| \|\|\|\|\|\| | |
| SBJCT: | 5324 | ACCAACCTCTCCTCCGTGGAGGCCTCCTATACAGTGGTACAAGATCAAGTGCGAAACAGC | 5383 |
| QUERY: | 5458 | TACCAGCTCTGTAATAATGGTACCCTGAGGGTGATGTATGCTAATGGGATGGGTATCAGC | 5517 |
| | | \|\|\|\|\|\|\|\|\|\|\| \|\|\|\|\|\|\|\| \|\|\|\|\|\| \|\|\|\|\|\|\|\|\|\| \|\| \|\| \|\| \|\|\|\| \| \|\|\|\|\| | |
| SBJCT: | 5384 | TACCAGCTCTGCAATAATGGAACCCTGCGGGTGATGTACGCCAACGGCATGGCTGTCAGC | 5443 |
| QUERY: | 5518 | TTCCACAGCGAGCCCCATGTCCTAGCGGGCACCATCACCCCCACCATTGGACGCTGCAAC | 5577 |
| | | \|\|\|\|\|\|\|\| \|\|\|\|\|\|\|\| \|\|\|\|\| \|\| \|\|\|\|\|\|\|\|\|\|\|\|\|\|\|\|\|\|\|\| \|\| \|\|\|\|\|\|\|\|\| | |
| SBJCT: | 5444 | TTCCACAGTGAGCCCCACGTCCTCGCAGGCACCATCACCCCCACCATCGGGCGCTGCAAC | 5503 |
| QUERY: | 5578 | ATCTCCCTGCCTATGGAGAATGGCTTAAACTCCATTGAGTGGCGCCTAAGAAAGGAACAG | 5637 |
| | | \|\|\|\|\| \|\|\|\|\| \|\|\|\|\|\|\|\|\|\|\|\| \| \|\|\|\|\|\|\|\| \|\|\|\|\|\|\|\|\|\|\| \|\| \|\|\|\|\|\|\|\|\| | |
| SBJCT: | 5504 | ATCTCTCTGCCCATGGAGAATGGCCTGAACTCCATCGAGTGGCGCCTGAGGAAGGAACAG | 5563 |
| QUERY: | 5638 | ATTAAAGGCAAAGTCACCATCTTTGGCAGGAAGCTCCGGGTCCATGGAAGAAATCTCTTG | 5697 |
| | | \|\| \|\|\|\|\|\|\|\|\|\|\|\|\|\|\|\|\|\|\|\|\|\|\| \|\|\|\|\|\|\|\| \|\|\|\|\|\|\|\| \|\|\|\|\| \|\|\|\|\|\| \|\| | |
| SBJCT: | 5564 | ATCAAAGGCAAAGTCACCATCTTTGGGAGGAAGCTTCGGGTCCACGGAAGGAATCTCCTG | 5623 |
| QUERY: | 5698 | TCCATTGACTATGATCGAAATATTCGGACTGAAAAGATCTATGATGACCACCGGAAGTTC | 5757 |
| | | \|\|\|\|\|\|\|\| \|\|\|\|\| \|\|\|\|\|\|\|\| \|\| \|\| \|\| \|\|\|\|\|\|\|\| \|\|\|\|\|\|\|\|\|\|\|\|\|\| \|\|\| | |
| SBJCT: | 5624 | TCCATTGATTATGACCGAAATATCCGTACGGAGAAGATCTACGATGACCACCGGAAATTC | 5683 |
| QUERY: | 5758 | ACCCTGAGGATCATTTATGACCAGGTGGGCCGCCCCTTCCTCTGGCTGCCCAGCAGCGGG | 5817 |
| | | \|\|\|\|\|\|\|\|\|\|\|\|\|\| \|\|\|\|\|\|\|\|\|\|\|\|\|\|\|\|\|\|\|\|\|\|\|\|\|\| \|\|\|\|\| \|\| \|\|\|\|\| \|\|\| | |
| SBJCT: | 5684 | ACCCTGAGGATCATCTATGACCAGGTGGGCCGCCCCTTCCTGTGGCTCCCGAGCAGTGGG | 5743 |
| QUERY: | 5818 | CTGGCAGCTGTCAACGTGTCATACTTCTTCAATGGGCGCCTGGCTGGGCTTCAGCGTGGG | 5877 |
| | | \|\|\|\|\|\|\|\| \|\|\|\|\| \|\| \|\| \|\|\|\|\|\|\|\|\|\|\|\|\|\|\|\|\|\| \|\|\|\| \|\| \|\| \|\|\|\|\| \|\|\| | |
| SBJCT: | 5744 | CTGGCAGCCGTCAATGTCTCCTACTTCTTCAATGGGCGCTTGGCCGGCCTCCAGCGAGGG | 5803 |
| QUERY: | 5878 | GCCATGAGCGAGAGGACAGACATCGACAAGCAAGGCCGCATCGTGTCCCGCATGTTCGCT | 5937 |
| | | \|\|\|\|\|\|\|\|\|\|\|\|\|\|\|\|\|\|\|\|\|\|\| \|\|\|\|\|\|\|\|\|\|\|\|\|\| \|\|\|\|\|\|\|\|\|\|\|\|\|\|\|\|\|\|\|\| | |
| SBJCT: | 5804 | GCCATGAGCGAGAGGACAGACATTGACAAGCAAGGCCGGATCGTGTCCCGCATGTTCGCC | 5863 |
| QUERY: | 5938 | GACGGGAAAGTGTGGAGCTACTCCTACCTTGACAAGTCCATGGTCCTCCTGCTTCAGAGC | 5997 |
| | | \|\|\|\|\|\|\|\|\|\|\| \|\|\|\|\| \|\| \|\|\|\|\| \|\|\|\|\|\|\|\|\|\|\|\|\|\|\|\|\|\|\|\| \|\|\|\|\| \|\|\|\|\|\| | |
| SBJCT: | 5864 | GACGGGAAAGTCTGGAGTTATTCCTATCTTGACAAGTCCATGGTCCTTCTGCTACAGAGC | 5923 |
| QUERY: | 5998 | CAACGTCAGTATATATTTGAGTATGACTCCTCTGACCGCCTCCTTGCCGTCACCATGCCC | 6057 |
| | | \|\|\|\|\|\|\|\|\|\|\| \|\|\|\|\|\|\|\| \|\|\|\|\|\|\|\|\|\|\| \|\| \|\|\|\|\|\|\| \|\| \|\|\|\|\| \|\|\|\|\|\| | |
| SBJCT: | 5924 | CAACGTCAGTACATATTTGAATATGACTCCTCCGATCGCCTCCACGCAGTCACTATGCCC | 5983 |
| QUERY: | 6058 | AGCGTGGCCCGGCACAGCATGTCCACACACACCTCCATCGGCTACATCCGTAATATTTAC | 6117 |
| | | \|\| \|\| \|\|\|\|\|\|\|\|\|\|\|\|\|\|\|\|\|\|\|\| \|\|\|\|\|\|\|\|\|\|\| \|\| \|\|\|\|\|\|\|\| \|\| \|\|\|\|\|\| | |
| SBJCT: | 5984 | AGTGTCGCCCGGCACAGCATGTCCACGCACACCTCCATTGGTTACATCCGAAACATTTAC | 6043 |
| QUERY: | 6118 | AACCCGCCTGAAAGCAATGCTTCGGTCATCTTTGACTACAGTGATGACGGCCGCATCCTG | 6177 |
| | | \|\|\|\|\| \|\| \|\|\|\|\|\|\|\|\|\|\| \|\|\|\|\|\|\|\|\|\|\|\|\|\|\|\|\|\|\|\|\|\|\|\|\|\|\|\|\|\|\|\|\|\|\|\|\|\| | |
| SBJCT: | 6044 | AACCCACCCGAAAGCAATGCATCGGTCATCTTTGACTACAGTGATGACGGCCGCATCCTA | 6103 |
| QUERY: | 6178 | AAGACCTCCTTTTTGGGCACCGGACGCCAGGTGTTCTACAAGTATGGGAAACTCTCCAAG | 6237 |
| | | \|\|\|\|\| \|\| \|\| \|\|\|\|\|\|\|\| \|\| \|\|\|\|\|\|\|\|\|\|\|\|\|\|\|\|\|\|\|\|\|\|\| \|\|\|\|\|\|\|\|\|\|\|\| | |
| SBJCT: | 6104 | AAGACATCTTTCTTGGGCACTGGGCGCCAGGTGTTCTACAAGTATGGAAAACTCTCCAAG | 6163 |
| QUERY: | 6238 | TTATCAGAGATTGTCTACGACAGTACCGCCGTCACCTTCGGGTATGACGAGACCACTGGT | 6297 |
| | | \|\|\|\|\|\|\|\|\|\|\| \|\|\|\|\|\|\|\|\|\|\| \|\| \|\|\|\|\|\|\|\|\|\|\| \|\|\|\|\|\|\|\|\|\|\|\|\|\|\|\|\| \|\|\| | |
| SBJCT: | 6164 | TTATCAGAGATAGTCTACGACAGCACAGCCGTCACCTTTGGGTATGACGAGACCACCGGT | 6223 |
| QUERY: | 6298 | GTCTTGAAGATGGTCAACCTCCAAAGTGGGGGCTTCTCCTGCACCATCAGGTACCGGAAG | 6357 |
| | | \|\|\| \|\|\|\|\|\|\|\|\|\|\|\|\| \|\|\|\|\|\|\|\|\|\|\|\|\|\|\|\|\|\|\|\|\|\|\| \|\|\|\|\|\|\|\|\|\|\|\|\|\| \|\|\| | |
| SBJCT: | 6224 | GTCCTGAAGATGGTCAATCTCCAAAGTGGGGGCTTCTCCTGTACCATCAGGTACCGAAAG | 6283 |
| QUERY: | 6358 | ATTGGCCCCCTGGTGGACAAGCAGATCTACAGGTTCTCCGAGGAAGGCATGGTCAATGCC | 6417 |
| | | \|\|\|\| \|\|\|\|\| \|\|\|\|\|\|\|\|\|\|\|\|\|\| \|\|\|\|\|\|\|\|\|\|\| \|\|\|\|\|\|\|\| \|\|\| \|\|\|\| \|\|\| | |
| SBJCT: | 6284 | GTTGGGCCCCTTGTGGACAAGCAGATTTACAGGTTCTCTGAGGAAGGAATGATCAACGCC | 6343 |
| QUERY: | 6418 | AGGTTTGACTACACCTATCATGACAACAGCTTCCGCATCGCAAGCATCAAGCCCGTCATA | 6477 |
| | | \|\|\|\|\|\|\|\| \|\| \|\|\|\|\|\|\|\| \|\|\|\|\| \|\|\|\|\|\|\|\|\|\|\| \|\| \|\|\|\|\|\|\|\| \|\|\|\|\|\|\|\| | |
| SBJCT: | 6344 | AGGTTTGATTATACCTATCACGACAATAGCTTCCGCATTGCCAGCATCAAACCCGTCATT | 6403 |
| QUERY: | 6478 | AGTGAGACTCCCCTCCCCGTTGACCTCTACCGCTATGATGAGATTTCTGGCAAGGTGGAA | 6537 |
| | | \|\| \|\|\|\|\|\|\|\|\|\|\| \|\| \|\|\|\|\|\|\|\|\|\|\|\|\|\|\|\|\|\|\|\| \|\|\|\|\|\|\|\| \|\|\|\|\|\|\|\|\|\|\|\| | |
| SBJCT: | 6404 | AGCGAGACTCCCCTTCCTGTTGACCTCTACCGCTATGACGAGATTTCCGGCAAGGTGGAA | 6463 |
| QUERY: | 6538 | CACTTTGGTAAGTTTGGAGTCATCTATTATGACATCAACCAGATCATCACCACTGCCGTG | 6597 |
| | | \|\|\|\|\| \|\| \|\|\|\|\|\|\|\| \|\|\|\|\|\|\| \|\| \|\|\|\|\|\|\|\|\|\|\|\|\|\|\|\|\|\|\|\|\|\|\|\|\|\|\|\|\|\| | |
| SBJCT: | 6464 | CACTTCGGCAAGTTTGGGGTCATCTACTACGACATCAACCAGATCATCACCACTGCCGTC | 6523 |
| QUERY: | 6598 | ATGACCCTCAGCAAACACTTCGACACCCATGGGCGGATCAAGGAGGTCCAGTATGAGATG | 6657 |
| | | \|\|\|\|\| \|\| \|\|\|\|\| \|\|\|\|\| \|\|\|\|\|\|\|\|\|\|\|\|\|\| \|\|\|\|\|\|\|\| \|\| \|\| \|\|\|\|\|\|\|\|\| | |
| SBJCT: | 6524 | ATGACGCTTAGCAAGCACTTTGACACCCATGGGCGCATCAAGGAAGTGCAATATGAGATG | 6583 |
| QUERY: | 6658 | TTCCGGTCCCTCATGTACTGGATGACGGTGCAATATGACAGCATGGGCAGGGTGATCAAG | 6717 |
| | | \|\|\|\|\|\|\|\|\|\|\|\|\|\|\|\|\|\|\|\|\|\|\|\|\|\| \|\|\|\|\|\|\|\|\|\|\|\|\|\| \|\|\|\|\| \|\|\|\|\| \|\|\|\|\|\| | |
| SBJCT: | 6584 | TTCCGGTCCCTCATGTACTGGATGACTGTGCAATATGACAGTATGGGTAGGGTCATCAAG | 6643 |
| QUERY: | 6718 | AGGGAGCTAAAACTGGGGCCCTATGCCAATACCACGAAGTACACCTATGACTACGATGGG | 6777 |
| | | \|\|\|\|\| \|\| \|\|\|\|\| \|\|\|\|\|\|\|\|\|\|\|\|\|\| \|\|\|\|\| \|\|\|\|\|\|\|\|\|\|\|\|\|\|\|\|\| \|\| \|\|\| | |
| SBJCT: | 6644 | AGGGAACTGAAACTAGGGCCCTATGCCAACACCACAAAGTACACCTATGACTATGACGGG | 6703 |
| QUERY: | 6778 | GACGGGCAGCTCCAGAGCGTGGCCGTCAATGACCGCCCGACCTGGCGCTACAGCTATGAC | 6837 |
| | | \|\|\|\|\| \|\|\|\|\|\|\|\|\|\|\| \|\|\|\|\|\|\|\|\|\|\|\|\|\|\|\|\| \|\| \|\|\|\|\|\|\|\|\|\|\| \|\|\|\|\|\|\|\|\| | |
| SBJCT: | 6704 | GACGGCCAGCTCCAGAGTGTGGCCGTCAATGACCGGCCTACCTGGCGCTATAGCTATGAC | 6763 |
| QUERY: | 6838 | CTTAATGGGAATCTCCACTTACTGAACCCAGGCAACAGTGTGCGCCTCATGCCCTTGCGC | 6897 |
| | | \|\| \|\|\|\|\|\|\|\| \|\| \|\|\| \| \|\| \|\|\|\|\|\|\|\| \|\|\|\|\|\|\| \|\|\|\|\|\|\|\|\|\|\|\|\|\| \|\|\| | |
| SBJCT: | 6764 | CTCAATGGGAACCTGCACCTTCTAAACCCAGGAAACAGTGCTCGCCTCATGCCCTTACGC | 6823 |
| QUERY: | 6898 | TATGACCTCCGGGATCGGATAACCAGACTCGGGGATGTGCAGTACAAAATTGACGACGAT | 6957 |
| | | \|\|\|\|\|\|\|\|\|\|\| \|\| \|\|\|\|\|\|\|\|\|\|\| \|\| \|\|\|\|\| \|\|\|\|\|\|\|\|\|\|\|\|\|\| \|\| \|\|\|\|\|\| | |
| SBJCT: | 6824 | TATGACCTCCGTGACCGGATAACCAGGCTAGGGGACGTGCAGTACAAAATCGATGACGAT | 6883 |
| QUERY: | 6958 | GGCTATCTGTGCCAGAGAGGGTCTGACATCTTCGAATACAATTCCAAGGGCCTCCTAACA | 7017 |
| | | \|\|\|\|\|\| \|\|\|\|\|\|\|\|\|\|\|\|\|\|\|\| \|\|\|\|\|\|\|\| \|\|\|\|\|\|\|\| \|\|\|\|\|\|\|\|\|\|\| \|\| \|\| | |
| SBJCT: | 6884 | GGCTATTTGTGCCAGAGAGGGTCAGACATCTTTGAATACAACTCCAAGGGCCTTCTGACG | 6943 |
| QUERY: | 7018 | AGAGCCTACAACAAGGCCAGCGGGTGGAGTGTCCAGTACCGCTATGATGGCGTAGGACGG | 7077 |
| | | \|\|\|\|\| \|\|\|\|\|\|\|\|\|\|\|\|\|\|\|\|\| \|\|\|\|\| \|\| \|\|\|\|\|\|\|\|\|\|\|\|\|\| \|\| \|\| \|\| \|\| | |
| SBJCT: | 6944 | AGAGCATACAACAAGGCCAGCGGATGGAGCGTGCAGTACCGCTATGACGGAGTGGGCCGC | 7003 |
| QUERY: | 7078 | CGGGCTTCCTACAAGACCAACCTGGGCCACCACCTGCAGTACTTCTACTCTGACCTCCAC | 7137 |
| | | \|\|\|\|\|\|\|\|\|\|\|\|\|\|\|\|\|\|\|\|\|\|\|\|\|\|\|\|\|\|\|\|\|\|\| \|\|\|\|\|\|\|\|\|\|\|\|\|\| \|\|\|\|\|\|\|\|\| | |
| SBJCT: | 7004 | CGGGCTTCCTACAAGACCAACCTGGGCCACCACCTACAGTACTTCTACTCCGACCTCCAC | 7063 |
| QUERY: | 7138 | AACCCGACGCGCATCACCCATGTCTACAATCACTCCAACTCGGAGATTACCTCACTGTAC | 7197 |
| | | \|\|\|\|\| \|\| \|\| \|\|\|\|\|\|\|\|\|\|\| \|\|\|\| \|\|\|\|\|\|\|\|\|\|\|\| \|\|\|\|\| \|\|\|\|\| \|\| \|\|\| | |
| SBJCT: | 7064 | AACCCCACACGTATCACCCATGTTTACAACCACTCCAACTCTGAGATCACCTCGCTCTAC | 7123 |
| QUERY: | 7198 | TACGACCTCCAGGGCCACCTCTTTGCCATGGAGAGCAGCAGTGGGGAGGAGTACTATGTT | 7257 |
| | | \|\| \|\|\|\|\|\|\|\|\|\|\|\|\|\|\|\|\| \|\|\|\|\|\|\|\|\|\|\|\|\|\|\|\|\| \|\|\|\|\| \|\| \|\| \|\|\|\|\|\|\|\| | |
| SBJCT: | 7124 | TATGACCTCCAGGGCCACCTATTTGCCATGGAGAGCAGTAGTGGTGAAGAATACTATGTC | 7183 |
| QUERY: | 7258 | GCCTCTGATAACACAGGGACTCCTCTGGCTGTGTTCAGCATCAACGGCCTCATGATCAAA | 7317 |
| | | \|\|\|\|\| \|\| \|\|\|\|\| \|\|\|\|\| \|\|\|\|\|\|\|\|\|\|\|\|\| \|\|\| \|\|\|\|\| \|\|\|\|\|\|\|\|\|\|\|\|\|\| | |
| SBJCT: | 7184 | GCCTCAGACAACACGGGGACCCCTCTGGCTGTGTACAGTATCAATGGCCTCATGATCAAG | 7243 |
| QUERY: | 7318 | CAGCTGCAGTACACGGCCTATGGGGAGATTTATTATGACTCCAACCCCGACTTCCAGATG | 7377 |
| | | \|\| \|\|\|\|\|\|\|\|\|\|\| \|\|\|\|\|\|\|\|\|\|\|\|\|\| \|\| \|\|\|\|\|\|\|\|\|\|\| \|\| \|\|\|\|\|\|\|\|\|\|\|\| | |
| SBJCT: | 7244 | CAACTGCAGTACACAGCCTATGGGGAGATCTACTATGACTCCAATCCAGACTTCCAGATG | 7303 |
| QUERY: | 7378 | GTCATTGGCTTCCATGGGGGACTCTATGACCCCCTGACCAAGCTGGTCCACTTCACTCAG | 7437 |
| | | \|\|\|\|\|\|\|\|\|\|\|\|\|\| \|\| \|\| \|\|\|\|\|\|\|\|\|\|\|\|\|\| \|\|\|\|\|\|\|\| \|\|\|\|\|\|\|\| \|\|\|\|\| | |
| SBJCT: | 7304 | GTCATTGGCTTCCACGGAGGCCTCTATGACCCCCTCACCAAGCTCGTCCACTTTACTCAA | 7363 |
| QUERY: | 7438 | CGTGATTATGATGTGCTGGCAGGACGATGGACCTCCCCAGACTATACCATGTGGAAAAAC | 7497 |
| | | \|\|\|\|\|\|\|\|\|\|\| \|\|\|\|\|\|\|\|\|\|\|\|\|\| \|\|\|\|\| \|\|\|\|\| \|\|\|\|\| \|\|\|\|\|\|\|\|\|\|\| \|\|\| | |
| SBJCT: | 7364 | CGTGATTATGACGTGCTGGCAGGACGGTGGACGTCCCCCGACTACACCATGTGGAGGAAC | 7423 |
| QUERY: | 7498 | GTGGGCAAGGAGCCGGCCCCCTTTAACCTGTATATGTTCAAGAGCAACAATCCTCTCAGC | 7557 |
| | | \|\|\|\|\|\|\|\|\|\|\|\|\|\| \|\|\|\|\|\|\|\| \|\|\|\|\|\|\|\| \|\|\|\|\|\|\|\|\|\| \|\|\|\|\|\|\|\|\|\|\|\| \|\|\| | |
| SBJCT: | 7424 | GTGGGCAAGGAGCCAGCCCCCTTCAACCTGTACATGTTCAAGAACAACAATCCTCTGAGC | 7483 |
| QUERY: | 7558 | AGTGAGCTAGATTTGAAGAACTACGTGACAGATGTGAAAAGCTGGCTTGTGATGTTTGGA | 7617 |
| | | \| \|\|\|\|\|\| \|\| \|\| \|\|\|\|\|\|\|\|\|\|\|\|\|\|\|\|\| \|\|\|\|\| \|\|\|\|\|\|\|\|\|\|\|\|\|\|\|\|\|\|\|\|\| | |
| SBJCT: | 7484 | AATGAGCTGGACTTAAAGAACTACGTGACAGACGTGAAGAGCTGGCTTGTGATGTTTGGA | 7543 |
| QUERY: | 7618 | TTTCAGCTTAGCAACATCATTCCTGGCTTCCCGAGAGCCAAAATGTATTTCGTGCCTCCT | 7677 |
| | | \|\|\|\|\|\|\|\| \|\|\|\|\|\|\|\|\|\|\|\|\|\|\|\|\| \|\|\|\|\|\|\|\|\|\|\|\|\|\|\|\|\|\|\|\|\|\|\| \|\|\|\|\|\|\|\| | |
| SBJCT: | 7544 | TTTCAGCTCAGCAACATCATTCCTGGATTCCCGAGAGCCAAAATGTATTTTGTGCCTCCC | 7603 |
| QUERY: | 7678 | CCCTATGAATTGTCAGAGAGTCAAGCAAGTGAGAATGGACAGCTCATTACAGGTGTCCAA | 7737 |
| | | \|\|\|\|\|\|\|\|\| \|\|\|\|\|\|\|\|\|\|\|\|\|\|\|\|\|\|\| \|\|\|\|\| \|\|\|\|\|\|\|\|\|\|\|\|\|\|\|\|\|\|\|\|\|\|\| | |
| SBJCT: | 7604 | CCCTATGAACTGTCAGAGAGTCAAGCAAGCGAGAACGGACAGCTCATTACAGGTGTCCAG | 7663 |
| QUERY: | 7738 | CAGACAACAGAGAGACATAACCAGGCCTTCATGGCTCTGGAAGGACAGGTCATTACTAAA | 7797 |
| | | \|\|\|\|\|\|\|\| \|\|\|\|\| \|\|\|\|\|\|\|\|\|\|\|\|\|\|\| \|\|\|\|\|\|\|\|\|\|\|\|\|\|\|\|\|\|\|\|\|\| \|\|\|\|\|\| | |
| SBJCT: | 7664 | CAGACAACTGAGAGGCATAACCAGGCCTTCCTGGCTCTGGAAGGACAGGTCATCACTAAA | 7723 |
| QUERY: | 7798 | AAGCTCCACGCCAGCATCCGAGAGAAAGCAGGTCACTGGTTTGCCACCACCACGCCCATC | 7857 |
| | | \|\|\|\|\|\|\|\| \|\|\|\|\|\|\|\|\|\|\|\|\|\|\|\|\|\|\|\|\|\|\| \|\|\|\|\|\|\|\|\|\|\| \|\|\|\|\|\|\|\| \|\|\|\|\|\| | |
| SBJCT: | 7724 | AAGCTCCATGCCAGCATCCGAGAGAAAGCAGGCCACTGGTTTGCTACCACCACACCCATC | 7783 |
| QUERY: | 7858 | ATTGGCAAAGGCATCATGTTTGCCATCAAAGAAGGGCGGGTGACCACGGGCGTGTCCAGC | 7917 |
| | | \|\| \|\|\|\|\|\|\|\|\|\|\|\|\|\|\|\|\|\|\|\|\|\|\|\|\|\|\|\|\|\|\|\|\|\|\|\|\|\|\|\|\|\|\|\| \|\| \|\|\|\|\| \|\|\| | |
| SBJCT: | 7784 | ATCGGCAAAGGCATCATGTTTGCCATCAAAGAAGGGCGGGTGACCACAGGAGTGTCTAGC | 7843 |
| QUERY: | 7918 | ATCGCCAGCGAAGATAGCCGCAAGGTGGCATCTGTGCTGAACAACGCCTACTACCTGGAC | 7977 |
| | | \|\|\|\|\|\|\|\| \|\| \|\| \|\|\|\|\|\|\|\|\|\|\| \|\|\|\|\| \|\|\| \|\|\|\|\|\|\| \|\|\|\|\|\|\|\|\| \| \|\|\| | |
| SBJCT: | 7844 | ATCGCCAGTGAGGACAGCCGCAAGGTAGCATCCGTGTTGAACAATGCCTACTACTTAGAC | 7903 |
| QUERY: | 7978 | AAGATGCACTACAGCATCGAGGGCAAGGACACCCACTACTTTGTGAAGATTGGCTCAGCC | 8037 |
| | | \|\|\|\|\|\|\|\|\|\|\|\|\|\|\|\|\|\|\|\|\|\|\|\|\|\|\|\|\|\|\|\| \|\|\|\|\|\|\|\|\|\|\|\|\|\|\|\|\| \|\|\| \| \|\| | |
| SBJCT: | 7904 | AAGATGCACTACAGCATCGAGGGCAAGGACACACACTACTTTGTGAAGATCGGCGCCGCG | 7963 |
| QUERY: | 8038 | GATGGCGACCTGGTCACACTAGGCACCACCATCGGCCGCAAGGTGCTAGAGAGCGGGGTG | 8097 |
| | | \|\|\|\|\| \|\|\|\|\|\|\|\|\|\|\| \|\|\|\|\| \|\|\|\|\|\|\|\| \|\| \|\|\|\|\|\|\|\|\|\|\| \|\|\|\|\| \|\|\|\|\|\| | |
| SBJCT: | 7964 | GATGGTGACCTGGTCACGCTAGGAACCACCATTGGGCGCAAGGTGCTGGAGAGTGGGGTG | 8023 |
| QUERY: | 8098 | AACGTGACCGTGTCCCAGCCCACGCTGCTGGTCAACGGCAGGACTCGAAGGTTCACGAAC | 8157 |
| | | \|\|\|\|\|\|\|\| \|\|\|\|\| \|\|\|\|\|\|\|\|\|\|\|\|\|\|\|\|\| \|\| \|\|\|\|\|\|\|\|\|\|\|\|\|\|\|\|\|\|\|\| \|\|\| | |
| SBJCT: | 8024 | AACGTGACGGTGTCACAGCCCACGCTGCTGGTGAATGGCAGGACTCGAAGGTTCACCAAC | 8083 |
| QUERY: | 8158 | ATTGAGTTCCAGTACTCCACGCTGCTGCTCAGCATCCGCTATGGCCTCACCCCCGACACC | 8217 |
| | | \|\|\|\|\|\|\|\|\|\|\|\|\|\|\|\|\|\|\|\|\|\|\|\|\|\|\|\|\|\|\|\| \|\|\|\|\|\|\|\| \|\|\|\|\|\|\|\|\|\|\|\|\|\|\|\|\| | |
| SBJCT: | 8084 | ATTGAGTTCCAGTACTCCACGCTGCTGCTCAGTATCCGCTACGGCCTCACCCCCGACACG | 8143 |
| QUERY: | 8218 | CTGGACGAAGAGAAGGCCCGCGTCCTGGACCAGGCGAGACAGAGGGCCCTGGGCACGGCC | 8277 |
| | | \|\|\|\|\|\|\|\|\|\|\| \|\|\|\|\|\|\|\|\|\|\|\|\|\|\|\|\|\|\|\| \|\|\| \|\|\|\|\|\|\| \|\|\|\|\|\|\|\| \|\| \|\|\| | |
| SBJCT: | 8144 | CTGGACGAAGAAAAGGCCCGCGTCCTGGACCAAGCGGGACAGAGAGCCCTGGGTACTGCC | 8203 |
| QUERY: | 8278 | TGGGCCAAGGAGCAGCAGAAAGCCAGGGACGGGAGAGAGGGGAGCCGCCTGTGGACTGAG | 8337 |
| | | \|\|\|\|\|\|\|\|\|\|\|\|\|\|\|\|\|\|\|\|\|\|\|\|\|\|\|\|\|\|\|\|\|\|\|\|\|\|\|\|\| \|\|\|\|\|\|\|\|\|\|\|\|\|\| \|\|\| | |
| SBJCT: | 8204 | TGGGCCAAGGAGCAGCAGAAAGCCAGGGACGGGAGAGAGGGCAGCCGCCTGTGGACGGAG | 8263 |
| QUERY: | 8338 | GGCGAGAAGCAGCAGCTTCTGAGCACCGGGCGCGTGCAAGGGTACGAGGGATATTACGTG | 8397 |
| | | \|\|\|\|\|\|\|\|\|\|\|\|\|\| \|\| \|\|\|\|\|\|\|\| \|\| \|\| \|\| \|\|\|\|\| \|\| \|\|\|\|\| \|\|\|\|\|\|\|\| | |
| SBJCT: | 8264 | GGCGAGAAGCAGCAACTCCTGAGCACGGGACGGGTACAAGGTTATGAGGGCTATTACGTA | 8323 |
| QUERY: | 8398 | CTTCCCGTGGAGCAATACCCAGAGCTTGCAGACAGTAGCAGCAACATCCAGTTTTTAAGA | 8457 |
| | | \|\|\|\|\| \|\|\|\|\| \|\| \|\|\|\|\| \|\|\|\|\| \|\|\|\|\|\|\|\|\|\|\|\|\|\|\|\|\|\|\|\|\|\|\|\|\|\| \|\|\|\|\|\| | |
| SBJCT: | 8324 | CTTCCGGTGGAACAGTACCCGGAGCTGGCAGACAGTAGCAGCAACATCCAGTTCTTAAGA | 8383 |
| QUERY: | 8458 | CAGAATGAGATGGGAAAGAGGTAACAAAATAATCTGCTGCCATTCCTTGTCTGAATGGCT | 8517 |
| | | \|\|\|\|\|\|\|\|\|\|\|\|\|\|\|\|\|\|\|\|\|\|\|\|\|\|\|\|\|\|\|\| \|\|\|\|\|\|\|\|\| \|\|\| \|\|\|\| \|\|\|\|\| | |
| SBJCT: | 8384 | CAGAATGAGATGGGAAAGAGGTAACAAAATAACCTGCTGCCACCTCTTCTCTGGGTGGCT | 8443 |
| QUERY: | 8518 | CAGCAGGAGTAACTGTTATCTCCTCTCCTAAGGAGATGAAGACCTAACAGGGGCACTGCG | 8577 |
| | | \|\|\|\|\|\|\|\|\| \|\|\|\|\|\| \| \|\|\|\|\|\|\|\|\|\|\|\|\|\|\|\|\| \|\|\|\|\|\|\|\|\|\|\| \|\|\|\|\|\|\|\|\| \| | |
| SBJCT: | 8444 | CAGCAGGAGCAACTGTGACCTCCTCTCCTAAGGAGACGAAGACCTAAC-GGGGCACTGAG | 8502 |
| QUERY: | 8578 | GCTGGGCTGCTTTAGGAGACCAAGTGGCAAGAAAGCTCACATTTTTTGAGTTCAAATGCT | 8637 |
| | | \|\| \|\|\|\|\|\|\|\|\|\|\|\|\|\| \|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 8503 | GCCGGGCTGCTTTAGGATCCCAAGTGGCAAGAAAGCTCACATTTTTTGAGTTCAAATGCT | 8562 |
| QUERY: | 8638 | ACTGTCCAAGCGAGAAGTCCCTCATCCTGAAGTAGACTAAAGCCCGGC 8685 | |
| | | \|\|\|\|\|\| \|\|\|\|\| \|\|\|\|\|\|\|\|\|\|\|\|\|\|\|\|\|\|\|\|\|\|\|\|\| \|\|\|\|\|\|\|\| | |
| SBJCT: | 8563 | ACTGTCTAAGCGCAAAGTCCCTCATCCTGAAGTAGACTAGAGCCCGGC 8610 | |

SCORE = 1570 BITS (792), EXPECT = 0.0
IDENTITIES = 1095/1196 (91%)
STRAND = PLUS/PLUS

| | | | |
|---|---|---|---|
| QUERY: | 270 | ATCTGGAATAATGGATGTAAAGGACCGGCGACACCGCTCTTTGACCAGAGGACGCTGTGG | 329 |
| | | \|\|\|\|\|\|\|\|\|\|\|\|\|\|\|\|\|\|\|\|\|\|\|\|\|\|\|\|\|\|\|\|\| \|\|\|\|\|\|\|\|\|\|\|\|\|\| \|\|\|\|\| \|\|\|\|\| | |
| SBJCT: | 103 | ATCTGGAATAATGGATGTAAAGGACCGGCGACATCGCTCTTTCACCAGGGGACGGTGTGG | 162 |
| QUERY: | 330 | CAAAGAGTGTCGCTACACAAGCTCCTCTCTGGACAGTGAGGACTGCCGGGTGCCCACACA | 389 |
| | | \|\|\|\|\|\|\|\|\|\|\|\|\|\|\|\|\|\| \|\|\|\|\|\|\|\|\|\|\|\|\|\|\|\|\|\|\|\|\|\|\|\|\|\|\|\|\| \|\|\|\|\|\|\|\| \|\| | |
| SBJCT: | 163 | CAAAGAGTGTCGCTACACCAGCTCCTCTCTGGACAGTGAGGACTGCCGTGTGCCCACTCA | 222 |
| QUERY: | 390 | GAAATCCTACAGCTCCAGTGAGACTCTGAAGGCCTATGACCATGACAGCAGGATGCACTA | 449 |
| | | \|\|\| \|\|\|\|\|\|\|\| \|\|\|\|\|\|\|\|\|\|\| \|\|\|\|\|\|\| \|\|\|\|\|\|\|\|\|\|\|\|\|\|\|\|\| \|\|\|\|\|\|\|\| | |
| SBJCT: | 223 | GAAGTCCTACAGTTCCAGTGAGACCTTGAAGGCTTATGACCATGACAGCAGAATGCACTA | 282 |
| QUERY: | 450 | TGGAAACCGAGTCACAGACCTCATCCACCGGGAGTCAGATGAGTTTCCTAGACAAGGAAC | 509 |
| | | \|\|\|\|\|\|\|\|\|\|\|\|\|\|\|\|\|\|\|\|\| \| \|\|\|\|\|\|\|\|\|\|\| \|\|\|\|\|\|\|\|\| \|\|\|\|\|\|\|\|\|\| \|\| | |
| SBJCT: | 283 | TGGAAACCGAGTCACAGACCTGGTGCACCGGGAGTCCGATGAGTTTTCTAGACAAGGGAC | 342 |
| QUERY: | 510 | CAACTTCACCCTTGCCGAACTGGGCATCTGTGAGCCCTCCCCACACCGAAGCGGCTACTG | 569 |
| | | \|\|\|\|\|\|\|\|\|\|\| \|\| \|\|\| \|\|\|\| \|\|\|\|\| \|\|\|\|\|\|\|\|\|\|\|\|\|\|\|\|\|\|\|\| \|\| \|\|\|\|\| | |
| SBJCT: | 343 | AAACTTCACCCTGGCAGAATTGGGAATCTGCGAGCCCTCCCCACACCGAAGTGGTTACTG | 402 |
| QUERY: | 570 | CTCCGACATGGGGATCCTTCACCAGGGCTACTCCCTTAGCACAGGGTCTGACGCCGACTC | 629 |
| | | \|\|\|\|\|\|\|\|\|\|\| \|\|\|\|\| \|\|\|\|\|\|\|\|\|\|\|\|\|\|\|\|\| \|\|\|\|\| \|\|\|\|\|\|\|\| \|\| \|\|\|\|\| | |
| SBJCT: | 403 | TTCCGACATGGGTATCCTCCACCAGGGCTACTCCCTGAGCACTGGGTCTGATGCAGACTC | 462 |
| QUERY: | 630 | CGACACCGAGGGAGGGATGTCTCCAGAACACGCCATCAGACTGTGGGGCAGAGGGATAAA | 689 |
| | | \|\|\|\|\|\|\|\|\|\|\|\|\|\|\|\|\|\|\|\|\|\|\|\|\|\|\|\|\|\|\|\|\|\| \|\|\|\|\|\|\|\|\|\|\|\| \|\|\|\|\|\|\|\|\|\| | |
| SBJCT: | 463 | GGACACCGAGGGAGGGATGTCTCCAGAACATGCCATCAGACTGTGGGGACGAGGGATAAA | 522 |
| QUERY: | 690 | ATCCAGGCGCAGTTCCGGCCTGTCCAGTCGTGAAAACTCGGCCCTTACCCTGACTGACTC | 749 |
| | | \|\|\|\|\|\|\|\|\|\|\|\| \|\| \|\|\| \|\|\|\|\|\|\| \|\| \|\| \|\|\|\|\|\|\|\|\|\|\|\|\|\| \|\|\|\|\|\|\|\|\|\|\| | |
| SBJCT: | 523 | ATCCAGGCGCAGCTCTGGCTTGTCCAGCCGCGAGAACTCGGCCCTTACTCTGACTGACTC | 582 |
| QUERY: | 750 | TGACAACGAAAACAAATCAGATGATGAGAACGGTCGTCCCATTCCACCTACATCCTCGCC | 809 |
| | | \|\|\|\|\|\| \|\|\|\|\| \|\|\|\|\| \|\|\|\|\| \|\| \|\| \|\|\|\|\|\|\|\|\|\|\|\|\|\|\|\|\|\|\|\|\|\|\|\|\|\|\| \| | |
| SBJCT: | 583 | TCACAATGAAAATAAATCGGATGACGACAATGGTCGTCCCATTCCACCTACATCCTCGTC | 642 |
| QUERY: | 810 | TAGTCTCCTCCCATCTGCTCAGCTGCCTAGCTCCCATAATCCTCCACCAGTTAGCTGCCA | 869 |
| | | \|\|\| \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 643 | TAGCCTCCTCCCATCTGCTCAGCTGCCTAGCTCCCATAATCCTCCACCAGTTAGCTGCCA | 702 |
| QUERY: | 870 | GATGCCATTGCTAGACAGCAACACCTCCCATCAAATCATGGACACCAACCCTGATGAGGA | 929 |
| | | \|\|\|\|\|\|\|\|\|\|\|\|\|\|\|\|\|\|\|\|\|\|\|\|\|\|\|\|\|\|\|\|\| \|\|\|\|\|\|\|\|\|\|\|\|\|\|\|\|\|\|\|\|\|\|\|\|\|\| | |
| SBJCT: | 703 | GATGCCATTGCTAGACAGCAACACCTCCCATCAGATCATGGACACCAACCCTGATGAGGA | 762 |
| QUERY: | 930 | ATTCTCCCCCAATTCATACCTGCTCAGAGCATGCTCAGGGCCCCAGCAAGCCTCCAGCAG | 989 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 763 | ATTCTCCCCCAATTCATACCTGCTCAGAGCATGCTCAGGGCCCCAGCAAGCCTCCAGCAG | 822 |
| QUERY: | 990 | TGGCCCTCCGAACCACCACAGCCAGTCGACTCTGAGGCCCCCTCTCCCACCCCCTCACAA | 1049 |
| | | \|\|\|\|\|\|\|\|\| \|\|\|\|\|\|\|\|\|\|\|\|\|\|\|\|\| \|\| \|\|\|\|\|\|\|\|\|\|\|\|\|\| \|\|\|\|\|\|\|\|\|\|\| \|\| | |
| SBJCT: | 823 | TGGCCCTCCAAACCACCACAGCCAGTCAACACTGAGGCCCCCTCTGCCACCCCCTCATAA | 882 |
| QUERY: | 1050 | CCACACGCTGTCCCATCACCACTCGTCCGCCAACTCCCTCAACAGGAACTCACTGACCAA | 1109 |
| | | \|\|\|\|\|\| \|\|\|\|\|\|\|\| \|\|\|\|\|\|\|\| \|\| \|\|\|\|\|\|\|\|\|\|\|\|\|\|\|\|\|\|\|\|\|\|\|\|\|\|\|\|\|\|\|\| | |
| SBJCT: | 883 | CCACACCCTGTCCCACCACCACTCCTCGGCCAACTCCCTCAACAGGAACTCACTGACCAA | 942 |
| QUERY: | 1110 | TCGGCGGAGTCAGATCCACGCCCCGGCCCCAGCGCCCAATGACCTGGCCACCACACCAGA | 1169 |
| | | \|\|\|\|\|\|\|\|\|\|\|\| \|\|\|\|\|\|\|\|\|\|\| \|\|\| \|\| \|\|\|\|\|\|\| \|\|\|\|\|\|\|\|\|\|\|\|\|\| \|\|\|\|\| | |
| SBJCT: | 943 | TCGGCGGAGTCAAATCCACGCCCCAGCTCCTGCGCCCAACGACCTGGCCACCACCCCAGA | 1002 |
| QUERY: | 1170 | GTCCGTTCAGCTTCAGGACAGCTGGGTGCTAAACAGCAACGTGCCACTGGAGACCCGGCA | 1229 |
| | | \|\|\| \|\|\|\|\|\|\|\| \|\|\|\|\| \|\|\|\|\|\|\|\|\|\|\| \|\|\|\|\| \|\|\|\|\| \|\|\|\|\|\|\|\|\|\|\| \|\|\|\|\| | |
| SBJCT: | 1003 | GTCTGTTCAGCTCCAGGATAGCTGGGTGCTGAACAGTAACGTCCCACTGGAGACTCGGCA | 1062 |
| QUERY: | 1230 | CTTCCTCTTCAAGACCTCCTCGGGGAGCACACCCTTGTTCAGCAGCTCTTCCCCGGGATA | 1289 |
| | | \|\|\|\|\|\| \|\|\|\|\| \|\| \|\| \|\| \|\| \|\|\|\|\|\|\|\|\| \|\|\|\|\|\|\|\|\|\|\|\|\|\|\|\| \|\|\|\|\|\|\|\| | |
| SBJCT: | 1063 | CTTCCTTTTCAAAACGTCGTCTGGAAGCACACCCCTGTTCAGCAGCTCTTCTCCGGGATA | 1122 |
| QUERY: | 1290 | CCCTTTGACCTCAGGAACGGTTTACACGCCCCCGCCCCGCCTGCTGCCCAGGAATACTTT | 1349 |
| | | \|\|\|\|\|\|\|\|\|\|\|\|\|\|\| \|\| \|\|\|\|\| \|\| \|\| \|\| \|\|\|\|\|\|\|\|\|\|\|\|\|\| \|\|\|\|\|\|\| \|\| | |
| SBJCT: | 1123 | CCCTTTGACCTCAGGGACCGTTTATACACCACCACCCCGCCTGCTGCCACGGAATACATT | 1182 |
| QUERY: | 1350 | CTCCAGGAAGGCTTTCAAGCTGAAGAAGCCCTCCAAATACTGCAGCTGGAAATGTGCTGC | 1409 |
| | | \|\|\|\|\|\|\|\|\|\|\|\| \|\|\|\|\|\|\|\|\|\|\|\|\|\| \|\|\|\|\|\|\|\|\|\|\|\|\|\|\|\|\| \|\|\|\|\|\|\|\|\|\|\|\|\|\| | |
| SBJCT: | 1183 | CTCCAGGAAGGCCTTCAAGCTGAAGAAACCCTCCAAATACTGCAGTTGGAAATGTGCTGC | 1242 |
| QUERY: | 1410 | CCTCTCCGCCATTGCCGCGGCCCTCCTCTTGGCTATTTTGCTGGCGTATTTCATAG 1465 | |
| | | \|\|\| \|\| \|\|\|\|\| \|\|\|\|\| \|\|\|\|\|\|\|\|\|\|\|\|\|\| \|\|\|\|\|\|\|\|\|\|\| \|\|\|\|\|\|\|\|\|\| | |
| SBJCT: | 1243 | CCTGTCTGCCATCGCCQCCGCCCTCCTCTTCGCCATTTTGCTGGCATATTTCATAG 1298 | |

SCORE = 1455 BITS (734), EXPECT = 0.0
IDENTITIES = 1000/1088 (91%), GAPS = 3/1088 (0%)
STRAND = PLUS/PLUS

| | | | |
|---|---|---|---|
| QUERY: | 1464 | AGTGCCCTGGTCGTTGAAAAACAGCAGCATAGACAGTGGTGAAGCAGAAGTTGGTCGGCG | 1523 |
| | | \|\|\|\|\|\|\|\|\|\|\|\| \|\|\|\|\|\|\|\|\|\|\|\|\|\|\|\|\|\|\|\|\|\|\|\|\|\| \|\|\|\|\|\|\|\|\|\|\|\|\|\|\|\|\|\|\|\| | |
| SBJCT: | 1420 | AGTGCCCTGCTCATTGAAAAACAGCAGCATAGACAGTGGCGAAGCAGAAGTTGGTCGGCG | 1479 |
| QUERY: | 1524 | GGTAACACAAGAAGTCCCACCAGGGGTGTTTTGGAGGTCACAAATTCACATCAGTCAGCC | 1583 |
| | | \|\|\| \|\|\|\|\| \|\|\|\|\|\|\|\|\|\|\|\|\|\|\|\|\|\|\|\|\|\|\|\|\|\|\|\|\| \|\| \|\|\|\|\|\|\|\|\|\|\|\|\|\|\|\|\| | |
| SBJCT: | 1480 | GGTGACACAGGAAGTCCCACCAGGGGTGTTTTGGAGGTCCCAGATTCACATCAGTCAGCC | 1539 |
| QUERY: | 1584 | CCAGTTCTTAAAGTTCAACATCTCCCTCGGGAAGGACGCTCTCTTTGGTGTTTACATAAG | 1643 |
| | | \|\| \|\|\|\|\|\|\|\|\|\|\|\|\|\|\|\|\|\|\|\|\|\|\| \|\| \|\|\|\|\| \|\| \|\|\|\|\| \|\|\|\|\| \|\| \|\|\|\|\| | |
| SBJCT: | 1540 | TCAATTCTTAAAGTTCAACATCTCCCTGGGCAAGGATGCCCTCTTCGGTGTCTATATAAG | 1599 |
| QUERY: | 1644 | AAGAGGACTTCCACCATCTCATGCCCAGTATGACTTCATGGAACGTCTGGACGGGAAGGA | 1703 |
| | | \|\|\|\|\|\|\|\| \|\|\|\|\| \|\|\|\|\|\|\|\|\|\|\|\|\|\|\|\|\|\|\|\|\|\|\|\|\|\|\|\|\| \|\|\|\|\| \|\| \|\|\|\|\| | |
| SBJCT: | 1600 | GAGAGGACTACCACCGTCTCATGCCCAGTATGACTTCATGGAACGCCTGGATGGAAAGGA | 1659 |
| QUERY: | 1704 | GAAGTGGAGTGTGGTTGAGTCTCCCAGGGAACGCCGGAGCATACAGACCTTGGTTCAGAA | 1763 |
| | | \|\|\| \|\|\|\|\| \|\|\|\|\| \|\|\|\|\| \|\|\|\|\|\|\|\|\|\|\|\|\|\|\|\|\|\|\|\| \|\|\|\|\| \|\|\|\| \|\|\|\|\| | |
| SBJCT: | 1660 | GAAATGGAGCGTGGTCGAGTCGCCCAGGGAACGCCGGAGCATCCAGACTCTGGTGCAGAA | 1719 |
| QUERY: | 1764 | TGAAGCCGTGTTTGTGCAGTACCTGGATGTGGGCCTGTGGCATCTGGCCTTCTACAATGA | 1823 |
| | | \|\| \|\| \|\|\|\|\|\|\|\|\|\|\|\|\|\|\| \|\|\|\|\|\|\|\|\|\|\|\|\|\|\|\|\|\|\| \|\|\|\|\|\|\|\|\|\|\|\|\|\|\|\|\| | |
| SBJCT: | 1720 | CGAGGCTGTGTTTGTGCAGTACTTGGATGTGGGCCTGTGGCACCTGGCCTTCTACAATGA | 1779 |
| QUERY: | 1824 | TGGAAAAGACAAAGAGATGGTTTCCTTCAATACTGTTGTCCTAGATTCAGTGCAGGACTG | 1883 |
| | | \|\| \|\| \|\|\|\|\| \|\|\|\|\|\|\|\| \|\|\|\|\|\|\|\| \|\|\|\|\|\|\|\|\| \|\|\|\|\|\|\|\|\|\|\|\|\|\|\|\|\|\|\| | |
| SBJCT: | 1780 | CGGCAAGGACAAGGAGATGGTCTCCTTCAACACTGTTGTCTTAGATTCAGTGCAGGACTG | 1839 |
| QUERY: | 1884 | TCCACGTAACTGCCATGGGAATGGTGAATGTGTGTCCGGGGTGTGTCACTGTTTCCCAGG | 1943 |
| | | \|\|\|\|\|\| \|\|\|\|\| \|\| \|\|\|\|\| \|\|\|\|\|\|\|\| \|\|\|\|\| \|\| \|\|\|\|\|\|\|\|\|\|\|\|\|\|\|\|\|\|\| | |
| SBJCT: | 1840 | TCCACGGAACTGTCACGGGAACGGTGAATGCGTGTCTGGACTGTGTCACTGTTTCCCAGG | 1899 |
| QUERY: | 1944 | ATTTCTAGGAGCAGACTGTGCTAAAGCTGCCTGCCCTGTCCTGTGCAGTGGGAATGGACA | 2003 |
| | | \|\|\| \|\|\|\|\| \|\|\|\|\|\|\|\|\|\|\|\|\|\|\|\|\|\|\|\|\|\|\|\|\|\|\|\|\| \|\|\|\|\|\|\|\| \|\| \|\|\|\|\|\|\|\| | |
| SBJCT: | 1900 | ATTCCTAGGTGCAGACTGTGCTAAAGCTGCCTGCCCTGTACTGTGCAGCGGAAATGGACA | 1959 |
| QUERY: | 2004 | ATATTCTAAAGGGACGTGCCAGTGCTACAGCGGCTGGAAAGGTGCAGAGTGCGACGTGCC | 2063 |
| | | \|\|\|\|\|\|\|\|\|\|\| \|\|\|\|\|\|\|\|\|\|\|\|\|\|\|\|\|\|\|\|\|\|\|\|\|\|\|\|\|\|\|\|\|\|\|\|\|\| \|\| \|\|\|\|\| | |
| SBJCT: | 1960 | GTATTCTAAAGGAACGTGCCAGTGCTACAGCGGCTGGAAAGGTGCAGAGTGTGATGTGCC | 2019 |
| QUERY: | 2064 | CATGAATCAGTGCATCGATCCTTCCTGCGGGGGCCACGGCTCCTGCATTGATGGGAACTG | 2123 |
| | | \|\|\|\|\| \|\| \|\| \|\|\|\|\|\|\|\|\|\|\|\|\|\| \|\|\|\|\|\|\|\| \|\|\|\|\|\|\|\|\|\|\|\|\|\|\|\|\|\|\|\|\|\|\| | |
| SBJCT: | 2020 | TATGAACCAATGTATCGATCCTTCCTGTGGGGGCCATGGCTCCTGCATTGATGGGAACTG | 2079 |
| QUERY: | 2124 | TGTCTGCTCTGCTGGCTACAAAGGCGAGCACTGTGAGGAAGTTGATTGCTTGGATCCCAC | 2183 |
| | | \|\| \|\| \|\|\|\|\|\|\|\|\|\|\|\|\| \|\|\|\|\|\|\|\|\|\|\|\|\|\|\|\|\|\|\|\|\|\|\|\|\|\|\|\|\|\|\|\|\|\|\| \|\| | |
| SBJCT: | 2080 | CGTGTGTGCTGCTGGCTACAAGGGCGAGCACTGTGAGGAAGTTGATTGCTTGGATCCTAC | 2139 |
| QUERY: | 2184 | CTGCTCCAGCCACGGAGTCTGTGTGAATGGAGAATGCCTGTGCAGCCCTGGCTGGGGTGG | 2243 |
| | | \|\|\|\|\|\|\|\|\|\|\|\| \|\| \|\|\|\|\|\|\|\|\|\|\|\|\|\|\|\|\| \|\| \|\| \|\|\|\|\|\|\|\| \|\|\|\|\|\|\|\|\|\|\| | |
| SBJCT: | 2140 | CTGCTCCAGCCATGGTGTCTGTGTGAATGGAGAGTGTCTATGCAGCCCCGGCTGGGGTGG | 2199 |
| QUERY: | 2244 | TCTGAACTGTGAGCTGGCGAGGGTCCAGTGCCCAGACCAGTGCAGTGGGCATGGCACGTA | 2303 |
| | | \|\|\| \|\|\|\|\|\|\|\|\|\|\|\|\|\|\|\|\|\|\|\|\|\|\|\|\|\|\|\|\|\|\|\|\|\|\|\|\|\| \|\|\|\|\|\|\|\|\|\|\|\|\|\| \|\| | |
| SBJCT: | 2200 | TCTCAACTGTGAGCTGGCGAGGGTCCAGTGCCCAGACCAGTGTAGTGGGCATGGCACTTA | 2259 |
| QUERY: | 2304 | CCTGCCTGACACGGGCCTCTGCAGCTGCGATCCCAACTGGATGGGTCCCGACTGCTCTGT | 2363 |
| | | \|\|\| \|\|\|\|\|\| \| \|\|\|\|\|\|\|\|\|\|\|\|\|\| \|\|\|\|\| \|\|\|\|\|\|\|\|\|\|\|\|\|\|\|\|\|\|\|\|\|\|\|\|\|\| | |
| SBJCT: | 2260 | CCTCCCTGACTCCGGCCTCTGCAGCTGTGATCCGAACTGGATGGGTCCCGACTGCTCTGT | 2319 |
| QUERY: | 2364 | TGAAGTGTGCTCAGTAGACTGTGGCACTCACGGCGTCTGCATCGGGGGAGCCTGCCGCTG | 2423 |
| | | \| \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 2320 | T---GTGTGCTCAGTAGACTGTGGCACTCACGGCGTCTGCATCGGGGGAGCCTGCCGCTG | 2376 |
| QUERY: | 2424 | TGAAGAGGGCTGGACAGGCGCAGCGTGTGACCAGCGCGTGTGCCACCCCCGCTGCATTGA | 2483 |
| | | \|\|\|\|\|\|\|\|\|\|\|\|\|\|\|\|\|\|\|\|\|\|\|\| \|\|\|\|\|\|\|\|\|\|\|\|\|\|\|\|\|\|\|\|\|\|\|\|\|\|\|\|\|\|\|\|\|\|\| | |
| SBJCT: | 2377 | TGAAGAGGGCTGGACAGGCGCAGCTTGTGACCAGCGCGTGTGCCACCCCCGCTGCATTGA | 2436 |
| QUERY: | 2484 | GCACGGGACCTGTAAAGATGGCAAATGTGAATGCCGAGAGGGCTGGAATGGTGAACACTG | 2543 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 2437 | GCACGGGACCTGTAAAGATGGCAAATGTGAATGCCGAGAGGGCTGGAATGGTGAACACTG | 2496 |
| QUERY: | 2544 | CACCATTG 2551 | |
| | | \|\|\|\|\|\|\|\| | |
| SBJCT: | 2497 | CACCATTG 2504 | |

SCORE = 105 BITS (53), EXPECT = 5E-19
IDENTITIES = 81/89 (91%), GAPS = 1/89 (1%)
STRAND = PLUS/PLUS

| | | | |
|---|---|---|---|
| QUERY: | 8711 | AACGAATGAATGAACAGACACACACAATGTTCCAAGTTCCCCTAAAATATGACCCACTTG | 8770 |
| | | \|\|\|\|\|\| \|\|\|\|\|\|\| \| \|\|\|\|\|\|\| \|\|\|\|\|\| \|\|\|\|\|\|\|\|\|\|\|\|\|\|\|\|\|\|\|\|\|\|\|\|\|\|\|\| | |
| SBJCT: | 8655 | AACGAACGAATGAAAACACACACAAAATGTTTCAAGTTCCCCTAAAATATGACCCACTTG | 8714 |
| QUERY: | 8771 | TTCTGCGTCT-ACGCAGAAAAGAGACGCA 8798 | |
| | | \|\|\| \|\|\|\|\|\| \| \|\|\|\|\|\|\|\|\|\|\|\|\|\|\|\| | |
| SBJCT: | 8715 | TTCCGGGTCTAAGGCAGAAAAGAGACGCA 8743 | |

SCORE = 48.1 BITS (24), EXPECT = 0.093
IDENTITIES = 30/32 (93%)
STRAND = PLUS/PLUS

| | | |
|---|---|---|
| QUERY: | 475 | CACCGGGAGTCAGATGAGTTTCCTAGACAAGG 506 |
| | | \|\|\|\|\|\|\|\|\|\|\| \|\|\|\|\|\|\|\|\| \|\|\|\|\|\|\|\|\|\| |
| SBJCT: | 7 | CACCGGGAGTCCGATGAGTTTTCTAGACAAGG 38 |

In this search it was also found that the FCTR3bcd and e nucleic acids had homology to three fragments of Rattus norvegicus neurestin alpha. It has 5498 of 6132 bases (89%) identical to bases 2527-8658, 1081 of 1196 bases (90%) identical to bases 123-1318, 996 of 1088 bases (91%) identical to bases 1440-2527 of Rattus norvegicus neurestin alpha (GenBank Acc:NM_{—}020088.1) (Table 3N).

TABLE 3N

BLASTN of FCTR3b, c, d, and e against Rattus norvegicus Neurestin alpha mRNA (SEQ
ID NO:66)

>GI|9910319|REF|NM 020088.1| RATTUS NORVEGICUS NEURESTIN ALPHA (LOC56762),
MRNA
LENGTH = 8689
SCORE = 7129 BITS (3596), EXPECT = 0.0
IDENTITIES = 5498/6132 (89%)
STRAND = PLUS/PLUS

| | | | |
|---|---|---|---|
| QUERY: | 2578 | GATGGCTGCCCTGACTTGTGCAACGGTAACGGGAGATGCACACTGGGTCAGAACAGCTGG | 2637 |
| | | \|\|\|\|\|\|\|\|\|\|\|\|\|\| \|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 2527 | GATGGCTGCCCTGATTTGTGCAACGGTAACGGGAGATGCACACTGGGTCAGAACAGCTGG | 2586 |
| QUERY: | 2638 | CAGTGTGTCTGCCAGACCGGCTGGAGAGGGCCCGGATGCAACGTTGCCATGGAAACTTCC | 2697 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| \|\|\| | |
| SBJCT: | 2587 | CAGTGTGTCTGCCAGACCGGCTGGAGAGGGCCCGGATGCAACGTTGCCATGGAAACCTCC | 2646 |
| QUERY: | 2698 | TGTGCTGATAACAAGGATAATGAGGGAGATGGCCTGGTGGATTGTTTGGACCCTGACTGC | 2757 |
| | | \|\| \|\|\|\|\|\|\|\|\|\|\|\|\|\|\|\|\|\|\|\|\|\|\|\|\|\|\|\|\|\|\|\|\|\|\|\|\|\| \|\| \|\|\|\|\|\|\|\|\|\|\|\|\|\| | |
| SBJCT: | 2647 | TGCGCTGATAACAAGGATAATGAGGGAGATGGCCTGGTGGACTGCCTGGACCCTGACTGC | 2706 |
| QUERY: | 2758 | TGCCTGCAGTCAGCCTGTCAGAACAGCCTGCTCTGCCGGGGGTCCCGGGACCCACTGGAC | 2817 |
| | | \|\|\|\|\| \|\|\|\|\|\|\|\|\|\|\|\|\|\|\|\|\|\|\|\|\|\|\|\|\|\|\|\|\| \|\|\|\|\|\|\|\| \|\|\|\|\|\|\|\| \|\|\|\|\| | |
| SBJCT: | 2707 | TGCCTCCAGTCAGCCTGTCAGAACAGCCTGCTCTGTCGGGGGTCTCGGGACCCCTTGGAC | 2766 |
| QUERY: | 2818 | ATCATTCAGCAGGGCCAGACGGATTGGCCCGCAGTGAAGTCCTTCTATGACCGTATCAAG | 2877 |
| | | \|\|\|\|\|\|\|\|\|\|\| \|\|\|\|\|\|\|\| \|\| \|\|\|\|\| \|\| \|\|\|\|\|\|\|\|\|\|\|\|\|\|\|\|\| \|\|\|\|\|\|\|\|\| | |
| SBJCT: | 2767 | ATCATTCAGCAAGGCCAGACAGACTGGCCTGCGGTGAAGTCCTTCTATGATCGCATCAAG | 2826 |
| QUERY: | 2878 | CTCTTGGCAGGCAAGGATAGCACCCACATCATTCCTGGAGAGAACCCTTTCAACAGCAGC | 2937 |
| | | \|\|\|\|\|\|\|\|\|\|\|\|\|\|\|\|\| \|\|\|\|\|\|\|\|\|\|\|\|\|\|\|\|\|\|\|\|\|\|\| \|\|\|\|\| \|\|\|\|\| \|\|\|\|\|\| | |
| SBJCT: | 2804 | CTCTTGGCAGGCAAGGACAGCACCCACATCATTCCTGGAGACAACCCCTTCAATAGCAGC | 2886 |
| QUERY: | 2938 | TTGGTTTCTCTCATCCGAGGCCAAGTAGTAACTACAGATGGAACTCCCCTGGTCGGTGTG | 2997 |
| | | \|\|\|\| \|\|\|\|\| \|\|\|\|\|\|\|\|\|\|\|\|\|\|\|\|\|\|\|\| \|\| \|\|\|\|\| \|\| \|\|\|\|\|\|\|\|\| \|\|\|\|\| | |
| SBJCT: | 2887 | CTGGTGTCTCTGATCCGAGGCCAAGTAGTAACCACGGATGGGACCCCCCTGGTGGGTGTG | 2946 |
| QUERY: | 2998 | AACGTGTCTTTTGTCAAGTACCCAAAATACGGCTACACCATCACCCGCCAGGATGGCACG | 3057 |
| | | \|\| \|\|\|\|\|\|\|\|\|\|\|\|\|\|\|\|\|\|\|\|\|\|\|\|\|\| \|\|\|\|\|\|\|\|\|\|\|\|\|\| \|\|\|\|\|\|\|\| \|\|\|\|\|\| | |
| SBJCT: | 2947 | AATGTGTCTTTTGTCAAGTACCCAAAATATGGCTACACCATCACTCGCCAGGACGGCACG | 3006 |
| QUERY: | 3058 | TTCGACCTGATCGCAAATGGAGGTGCTTCCTTGACTCTACACTTTGAGCGAGCCCCGTTC | 3117 |
| | | \|\| \|\|\|\|\|\|\|\| \|\| \|\|\|\|\| \|\| \|\| \|\|\|\|\|\|\|\|\|\| \|\|\|\|\|\|\|\|\|\|\|\|\|\|\|\|\| \|\|\| | |
| SBJCT: | 3007 | TTTGACCTGATTGCCAATGGGGGCTCTGCCTTGACTCTTCACTTTGAGCGAGCCCCTTTC | 3066 |
| QUERY: | 3118 | ATGAGCCAGGAGCGCACTGTGTGGCTGCCGTGGAACAGCTTTTACGCCATGGACACCCTG | 3177 |
| | | \|\|\|\|\|\|\| \|\|\|\|\|\|\|\|\| \|\| \|\|\|\| \|\|\|\|\|\|\|\|\|\|\|\|\|\|\| \|\| \|\|\|\|\|\|\|\|\|\|\|\|\|\|\| | |
| SBJCT: | 3067 | ATGAGCCGGGAGCGCACAGTATGGCCGCCGTGGAACAGCTTCTATGCCATGGACACCCTG | 3126 |
| QUERY: | 3178 | GTGATGAAGACCGAGGAGAACTCCATCCCCAGCTGTGACCTCAGTGGCTTTGTCCGGCCT | 3237 |
| | | \|\| \|\|\|\|\|\|\|\| \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 3127 | GTAATGAAGACGGAGGAGAACTCCATCCCCAGCTGTGACCTCAGTGGCTTTGTCCGGCCT | 3186 |
| QUERY: | 3238 | GATCCAATCATCATCTCCTCCCCACTGTCCACCTTCTTTAGTGCTGCCCCTGGGCAGAAT | 3297 |
| | | \|\|\|\|\| \|\|\|\|\|\|\|\|\|\|\|\|\|\| \|\| \|\|\|\|\|\|\|\|\|\|\|\|\|\| \|\| \|\|\| \|\|\|\|\|\| \| \|\|\| | |
| SBJCT: | 3187 | GATCCGATCATCATCTCCTCTCCTCTGTCCACCTTCTTCAGCGCTTCCCCTGCGGCGAAC | 3246 |
| QUERY: | 3298 | CCCATCGTGCCTGAGACCCAGGTTCTTCATGAAGAAATCGAGCTCCCTGGTTCCAATGTG | 3357 |
| | | \|\|\|\|\| \|\|\|\|\|\|\|\|\|\|\|\|\|\|\|\|\|\|\|\|\|\|\|\|\|\| \|\| \|\|\|\|\|\|\|\|\|\|\|\|\|\| \|\|\|\| \|\|\| | |
| SBJCT: | 3247 | CCCATTGTGCCTGAGACCCAGGTTCTTCATGAGGAGATCGAGCTCCCTGGCACCAACGTG | 3306 |
| QUERY: | 3358 | AAACTTCGCTATCTGAGCTCTAGAACTGCAGGGTACAAGTCACTGCTGAAGATCACCATG | 3417 |
| | | \|\| \|\| \|\| \|\| \|\| \|\|\|\|\| \|\|\|\|\| \|\|\|\|\|\|\|\|\|\|\|\|\|\|\|\|\|\|\|\|\|\|\|\|\|\|\|\|\|\|\|\|\| | |
| SBJCT: | 3307 | AAGCTCCGTTACCTCAGCTCCAGAACAGCAGGGTACAAGTCACTGCTGAAGATCACCATG | 3366 |
| QUERY: | 3418 | ACCCAGTCCACAGTGCCCCTGAACCTCATTAGGGTTCACCTGATGGTGGCTGTCGAGGGG | 3477 |
| | | \|\|\|\|\|\|\|\|\|\|\| \|\|\|\|\|\| \|\|\|\|\|\|\|\|\|\| \|\|\|\|\|\|\|\| \|\|\|\|\|\|\| \|\| \|\| \|\|\|\|\| | |
| SBJCT: | 3367 | ACCCAGTCCACGGTGCCCTTGAACCTCATCCGGGTTCACTTGATGGTTGCCGTGGAGGGG | 3426 |
| QUERY: | 3478 | CATCTCTTCCAGAAGTCATTCCAGGCTTCTCCCAACCTGGCCTCCACCTTCATCTGGGAC | 3537 |
| | | \|\|\|\|\|\|\|\|\|\|\|\|\|\|\|\|\| \|\|\|\|\|\|\|\|\|\|\|\|\|\|\|\|\|\|\|\|\|\|\|\|\| \|\|\| \|\|\|\|\|\|\|\|\|\|\|\| | |
| SBJCT: | 3427 | CATCTCTTCCAGAAGTCGTTCCAGGCTTCTCCCAACCTGGCCTACACATTCATCTGGGAC | 3486 |
| QUERY: | 3538 | AAGACAGATGCGTATGGCCAAAGGGTGTATGGACTCTCAGATGCTGTTGTGTCTGTCGGG | 3597 |
| | | \|\|\|\|\|\|\|\| \|\| \|\|\|\|\|\|\|\|\|\|\|\|\|\| \|\|\|\|\| \|\| \|\| \|\|\|\|\|\|\|\|\|\|\|\|\|\|\|\|\| \|\|\| | |
| SBJCT: | 3487 | AAGACAGACGCTTATGGCCAAAGGGTTTATGGCCTATCGGATGCTGTTGTGTCTGTTGGA | 3546 |
| QUERY: | 3598 | TTTGAATATGAGACCTGTCCCAGTCTAATTCTCTGGGAGAAAAGGACAGCCCTCCTTCAG | 3657 |
| | | \|\|\|\|\|\|\|\|\|\|\|\|\|\|\|\|\| \|\|\|\|\|\|\|\| \|\| \|\| \|\|\|\|\| \|\|\|\|\|\|\|\|\|\|\|\|\|\| \|\|\|\|\|\| | |
| SBJCT: | 3547 | TTTGAATATGAGACCTGCCCCAGTCTCATCCTGTGGGAAAAAAGGACAGCCCTACTTCAA | 3606 |
| QUERY: | 3658 | GGATTCGAGCTGGACCCCTCCAACCTCGGTGGCTGGTCCCTAGACAAACACCACATCCTC | 3717 |
| | | \|\|\|\|\|\|\|\|\|\|\|\|\|\|\|\|\| \|\|\|\|\|\|\|\| \|\|\|\|\|\|\|\|\|\|\|\|\|\| \|\| \|\| \|\|\|\|\|\|\| \|\|\|\| | |
| SBJCT: | 3607 | GGATTCGAGCTGGACCCTTCCAACCTTGGTGGCTGGTCCCTGGATAAGCACCACACCCTC | 3666 |
| QUERY: | 3718 | AATGTTAAAAGTGGAATCCTACACAAAGGCACTGGGGAAAACCAGTTCCTGACCCAGCAG | 3777 |
| | | \|\|\|\|\| \|\|\|\|\| \|\|\|\|\| \|\|\|\| \|\|\|\|\|\|\|\|\| \|\|\|\|\| \|\|\|\|\|\|\|\|\|\|\|\|\|\|\|\|\|\|\|\|\| | |
| SBJCT: | 3667 | AATGTGAAAAGCGGAATACTACTCAAAGGCACAGGGGAGAACCAGTTCCTGACCCAGCAG | 3726 |
| QUERY: | 3778 | CCTGCCATCATCACCAGCATCATGGGCAATGGTCGCCGCCGGAGCATTTCCTGTCCCAGC | 3837 |
| | | \|\| \|\|\|\|\|\|\|\|\|\|\|\|\|\|\|\|\|\|\|\|\|\|\| \|\| \|\|\|\|\|\|\|\|\| \| \|\|\|\|\| \|\|\|\|\|\|\|\|\|\|\|\| | |
| SBJCT: | 3727 | CCCGCCATCATCACCAGCATCATGGGTAACGGTCGCCGCAGAAGCATCTCCTGTCCCAGC | 3786 |
| QUERY: | 3838 | TGCAACGGCCTTGCTGAAGGCAACAAGCTGCTGGCCCCAGTGGCTCTGGCTGTTGGAATC | 3897 |
| | | \|\|\|\|\| \|\|\|\|\|\|\|\|\|\|\|\|\|\|\|\|\|\|\|\| \|\|\| \|\|\|\|\|\|\| \|\|\|\|\| \|\|\|\|\|\|\|\| \|\| \|\|\| | |
| SBJCT: | 3787 | TGCAATGGCCTTGCTGAAGGCAACAAACTGTTGGCCCCCGTGGCCCTGGCTGTGGGGATC | 3846 |
| QUERY: | 3898 | GATGGGAGCCTCTATGTGGGTGACTTCAATTACATCCGACGCATCTTTCCCTCTCGAAAT | 3957 |
| | | \|\|\|\|\|\|\|\|\|\|\|\|\| \|\|\| \|\|\|\|\|\|\|\|\|\|\|\|\|\| \|\|\|\|\| \|\|\|\|\|\|\|\| \|\| \|\|\|\|\|\|\|\|\| | |
| SBJCT: | 3847 | GATGGGAGCCTCTTTGTCGGTGACTTCAATTATATCCGGCGCATCTTCCCTTCTCGAAAT | 3906 |
| QUERY: | 3958 | GTGACCAGCATCTTGGAGTTACGAAATAAAGAGTTTAAACATAGCAACAACCCAGCACAC | 4017 |
| | | \|\|\|\|\|\|\|\| \|\|\|\|\|\|\|\|\|\|\|\|\|\|\|\|\|\|\|\|\|\|\|\|\|\|\|\|\|\|\|\|\|\|\|\|\|\|\|\| \|\|\|\|\| \|\|\|\| | |
| SBJCT: | 3907 | GTGACCAGTATCTTGGAGTTACGAAATAAAGAGTTTAAACATAGCAACAGCCCAGGACAC | 3966 |
| QUERY: | 4018 | AAGTACTACTTGGCAGTGGACCCCGTGTCCGGCTCGCTCTACGTGTCCGACACCAACAGC | 4077 |
| | | \|\|\|\|\|\|\|\|\|\|\|\|\|\| \|\|\|\|\|\|\|\| \|\|\| \| \|\|\|\|\|\|\|\|\|\|\| \|\| \|\| \|\|\|\|\|\|\|\|\|\|\| | |
| SBJCT: | 3944 | AAGTACTACTTGGCTGTGGACCCTGTGACTGGCTCGCTCTATGTCTCTGACACCAACAGT | 4026 |
| QUERY: | 4078 | AGGAGAATCTACCGCGTCAAGTCTCTGAGTGGAACCAAAGACCTGGCTGGGAATTCGGAA | 4137 |
| | | \| \| \|\|\|\|\|\|\|\| \|\|\|\|\|\|\|\|\|\|\| \|\| \|\|\| \|\|\|\|\|\|\|\|\|\|\|\|\|\|\|\|\|\|\|\|\|\|\|\|\|\| | |
| SBJCT: | 4027 | CGCCGGATCTACCGAGTCAAGTCTCTAAGCGGAGCCAAAGACCTGGCTGGGAATTCGGAA | 4086 |
| QUERY: | 4138 | GTTGTGGCAGGGACGGGAGAGCAGTGTCTACCCTTTGATGAAGCCCGCTGCGGGGATGGA | 4197 |
| | | \|\|\|\|\|\|\|\| \|\|\|\|\| \|\| \|\| \|\| \|\|\|\|\|\|\|\|\|\|\|\|\|\|\|\|\|\|\|\|\|\|\|\|\|\| \|\|\|\|\|\|\|\| | |
| SBJCT: | 4087 | GTTGTGGCCGGGACTGGCGAACAATGTCTACCCTTTGATGAAGCCCGCTGTGGGGATGGC | 4146 |
| QUERY: | 4198 | GGGAAGGCCATAGATGCAACCCTGATGAGCCCGAGAGGTATTGCAGTAGACAAGAATGGG | 4257 |
| | | \|\|\|\|\|\|\|\| \| \|\|\|\|\| \|\|\|\|\|\|\|\|\|\|\|\|\|\| \|\|\|\|\|\|\|\|\|\|\|\|\|\|\|\|\|\|\|\|\|\|\| \|\|\| | |
| SBJCT: | 4147 | GGGAAGGCTGTGGATGCCACCCTGATGAGCCCTAGAGGTATTGCAGTAGACAAGAACGGG | 4206 |
| QUERY: | 4258 | CTCATGTACTTTGTCGATGCCACCATGATCCGGAAGGTTGACCAGAATGGAATCATCTCC | 4317 |
| | | \|\| \|\|\|\|\| \|\|\|\|\| \|\|\|\|\|\|\|\|\|\|\|\|\|\|\|\|\|\|\|\|\|\|\| \|\|\|\|\| \|\|\|\|\|\|\|\|\|\|\|\|\|\|\| | |
| SBJCT: | 4207 | CTTATGTATTTTGTTGATGCCACCATGATCCGGAAGGTCGACCAAAATGGAATCATCTCC | 4266 |
| QUERY: | 4318 | ACCCTGCTGGGCTCCAATGACCTCACTGCCGTCCGGCCGCTGAGCTGTGATTCCAGCATG | 4377 |
| | | \|\|\|\|\|\|\|\|\|\|\|\|\|\|\|\|\|\|\|\|\|\|\|\|\|\| \|\| \|\|\|\|\| \|\| \|\|\|\|\|\|\|\|\|\|\| \|\| \|\|\|\|\|\| | |
| SBJCT: | 4267 | ACCCTGCTGGGCTCCAATGACCTCACAGCTGTCCGACCACTGAGCTGTGACTCTAGCATG | 4326 |
| QUERY: | 4378 | GATGTAGCCCAGGTTCGTCTGGAGTGGCCAACAGACCTTGCTGTCAATCCCATGGATAAC | 4437 |
| | | \|\| \|\| \|\|\|\|\|\|\|\| \|\|\|\|\| \|\| \|\|\|\|\| \|\|\|\|\|\|\|\|\|\|\| \|\|\|\|\| \|\|\|\|\|\|\|\| \|\| | |
| SBJCT: | 4327 | GACGTGGCCCAGGTCCGTCTAGAATGGCCGACAGACCTTGCGGTCAACCCCATGGACAAT | 4386 |
| QUERY: | 4438 | TCCTTGTATGTTCTAGAGAACAATGTCATCCTTCGAATCACCGAGAACCACCAAGTCAGC | 4497 |
| | | \|\|\| \|\|\|\| \|\| \|\| \|\|\|\|\|\|\|\| \|\|\|\|\|\|\|\| \|\| \|\|\|\|\|\|\|\|\|\|\| \|\|\|\|\| \|\|\|\|\|\| | |
| SBJCT: | 4387 | TCCCTGTACGTCCTGGAGAACAACGTCATCCTGCGGATCACCGAGAATCACCAGGTCAGC | 4446 |
| QUERY: | 4498 | ATCATTGCGGGACGCCCCATGCACTGCCAAGTTCCTGGCATTGACTACTCACTCAGCAAA | 4557 |
| | | \|\|\|\|\| \|\|\|\|\|\|\|\| \|\|\|\|\|\|\|\|\|\|\|\|\|\| \|\|\|\|\| \|\|\|\|\| \|\|\|\|\|\|\|\| \|\|\|\|\|\|\|\| | |
| SBJCT: | 4447 | ATCATCGCGGGACGGCCCATGCACTGCCAGGTTCCCGGCATCGACTACTCGCTCAGCAAG | 4506 |
| QUERY: | 4558 | CTAGCCATTCACTCTGCCCTGGAGTCAGCCAGTGCCATTGCCATTTCTCACACTGGGGTC | 4617 |
| | | \|\| \|\|\|\|\| \|\|\|\|\|\|\|\| \|\|\|\|\|\|\|\|\|\|\|\|\|\| \|\|\|\|\| \|\|\|\|\|\|\|\|\|\|\|\|\|\|\|\|\|\|\|\|\| | |
| SBJCT: | 4507 | CTCGCCATCCACTCTGCTCTGGAGTCAGCCAGCGCCATCGCCATTTCTCACACTGGGGTG | 4566 |
| QUERY: | 4618 | CTCTACATCACTGAGACAGATGAGAAGAAGATTAACCGTCTACGCCAGGTAACAACCAAC | 4677 |
| | | \|\|\|\|\|\|\|\|\|\|\| \|\|\|\|\| \|\| \|\|\|\|\|\|\|\|\|\|\| \|\|\|\|\| \|\|\|\|\|\|\|\| \|\| \|\| \|\|\|\|\|\| | |
| SBJCT: | 4567 | CTCTACATCACCGAGACGGACGAGAAGAAGATCAACCGCCTACGCCAAGTCACCACCAAC | 4626 |
| QUERY: | 4678 | GGGGAGATCTGCCTTTTAGCTGGGGCAGCCTCGGACTGCGACTGCAAAAACGATGTCAAT | 4737 |
| | | \|\| \|\|\|\|\|\|\|\|\|\|\| \|\|\|\|\| \|\|\|\|\|\|\|\|\|\|\| \|\|\|\|\| \|\|\|\|\|\|\|\|\|\|\| \|\| \|\|\|\|\| | |
| SBJCT: | 4627 | GGAGAGATCTGCCTCTTAGCCGGGGCAGCCTCAGACTGTGACTGCAAAAATGACGTCAAC | 4686 |
| QUERY: | 4738 | TGCAACTGCTATTCAGGAGATGATGCCTACGCGACTGATGCCATCTTGAATTCCCCATCA | 4797 |
| | | \|\|\|\| \|\|\|\|\|\|\|\|\| \|\|\|\|\|\|\|\| \|\| \|\|\|\|\| \|\| \|\|\|\|\|\|\|\|\|\|\|\|\|\| \|\|\|\|\| \|\| | |
| SBJCT: | 4687 | TGCATCTGCTATTCGGGAGATGACGCATACGCCACGGATGCCATCTTGAACTCCCCGTCC | 4746 |
| QUERY: | 4798 | TCCTTAGCTGTAGCTCCAGATGGTACCATTTACATTGCAGACCTTGGAAATATTCGGATC | 4857 |
| | | \|\|\|\|\|\|\|\|\|\|\| \|\|\|\|\| \|\|\|\|\| \|\|\|\|\| \|\|\|\|\| \|\|\|\|\|\|\|\| \|\| \|\|\|\|\| \|\|\|\|\|\| | |
| SBJCT: | 4747 | TCCTTAGCTGTGGCTCCGGATGGCACCATCTACATCGCAGACCTCGGGAATATCCGGATC | 4806 |
| QUERY: | 4858 | AGGGCGGTCAGCAAGAACAAGCCTGTTCTTAATGCCTTCAACCAGTATGAGGCTGCATCC | 4917 |
| | | \|\|\|\|\|\|\|\|\|\|\|\|\|\| \|\|\|\|\| \|\|\|\|\|\|\|\|\|\|\| \|\| \|\|\|\|\|\|\|\|\|\|\|\|\|\|\|\|\|\|\|\| \|\| | |
| SBJCT: | 4807 | AGGGCGGTCAGCAAAAACAAACCTGTTCTTAACGCGTTCAACCAGTATGAGGCTGCGTCT | 4866 |
| QUERY: | 4918 | CCCGGAGAGCAGGAGTTATATGTTTTCAACGCTGATGGCATCCACCAATACACTGTGAGC | 4977 |
| | | \|\| \|\|\|\|\| \|\|\|\|\| \| \|\| \|\| \|\|\|\|\|\|\|\| \|\|\|\|\| \|\|\|\|\| \|\| \|\|\|\|\| \|\|\|\|\| | |
| SBJCT: | 4867 | CCGGGAGAACAGGAACTGTACGTGTTCAACGCCGATGGTATCCATCAGTACACCGTGAGC | 4926 |
| QUERY: | 4978 | CTGGTGACAGGGGAGTACTTGTACAATTTCACATATAGTACTGACAATGATGTCACTGAA | 5037 |
| | | \|\|\|\|\|\|\|\| \|\|\|\|\|\|\|\|\|\|\| \|\|\|\|\|\|\|\|\|\|\| \|\| \|\| \|\|\|\|\|\|\|\|\|\|\|\|\|\|\|\| \|\| | |
| SBJCT: | 4927 | CTGGTGACCGGGGAGTACTTATACAATTTCACCTACAGCGCTGACAATGATGTCACCGAG | 4986 |
| QUERY: | 5038 | TTGATTGACAATAATGGGAATTCCCTGAAGATCCGTCGGGACAGCAGTGGCATGCCCCGT | 5097 |
| | | \|\|\|\|\|\|\|\|\|\|\| \|\| \|\|\|\|\|\|\|\|\|\|\| \|\|\|\|\|\|\|\| \|\|\|\|\|\|\|\|\|\|\|\|\|\|\|\|\|\|\|\|\|\|\| | |
| SBJCT: | 4987 | TTGATTGACAACAACGGGAATTCCCTAAAGATCCGCCGGGACAGCAGTGGCATGCCCCGA | 5046 |
| QUERY: | 5098 | CACCTGCTCATGCCTGACAACCAGATCATCACCCTCACCGTGGGCACCAATGGAGGCCTC | 5157 |
| | | \|\|\|\|\|\|\|\|\|\|\|\|\|\|\|\|\| \|\| \|\|\|\|\|\|\|\|\|\|\|\|\|\| \|\| \|\|\|\|\|\|\|\|\|\|\| \|\|\|\|\|\|\|\|\| | |
| SBJCT: | 5047 | CACCTGCTCATGCCTGATAATCAGATCATCACCCTTACGGTGGGCACCAACGGAGGCCTC | 5106 |
| QUERY: | 5158 | AAAGTCGTGTCCACACAGAACCTGGAGCTTGGTCTCATGACCTATGATGGCAACACTGGG | 5217 |
| | | \|\|\|\| \|\|\|\|\|\| \|\| \|\|\|\|\|\|\|\|\|\|\|\|\|\| \|\| \|\|\|\|\|\|\|\| \|\|\|\|\|\|\|\| \|\|\|\|\|\|\|\| | |
| SBJCT: | 5107 | AAAGCCGTGTCAACGCAGAACCTGGAGCTGGGCCTCATGACTTATGATGGGAACACTGGA | 5166 |
| QUERY: | 5218 | CTCCTGGCCACCAAGAGCGATGAAACAGGATGGACGACTTTCTATGACTATGACCACGAA | 5277 |
| | | \|\|\|\|\| \|\|\|\|\|\|\|\|\|\|\|\|\|\|\|\|\|\|\|\| \|\|\|\|\|\|\|\| \|\|\|\|\| \|\|\|\|\|\|\|\|\|\|\|\|\|\|\|\|\| | |
| SBJCT: | 5167 | CTCCTAGCCACCAAGAGCGATGAAACCGGATGGACAACTTTTTATGACTATGACCACGAG | 5226 |
| QUERY: | 5278 | GGCCGCCTGACCAACGTGACGCGCCCCACGGGGGTGGTAACCAGTCTGCACCGGGAAATG | 5337 |
| | | \|\|\|\|\| \|\|\|\|\|\|\|\| \|\|\|\|\| \|\|\|\|\|\|\|\|\|\|\|\|\|\|\|\|\| \|\|\|\|\| \|\|\|\|\|\|\|\|\|\|\|\|\|\|\| | |
| SBJCT: | 5227 | GGCCGTCTGACCAATGTGACTCGCCCCACGGGGGTGGTGACCAGCCTGCACCGGGAAATG | 5286 |
| QUERY: | 5338 | GAGAAATCTATTACCATTGACATTGAGAACTCCAACCGTGATGATGACGTCACTGTCATC | 5397 |
| | | \|\|\|\|\|\|\|\| \|\| \|\|\| \|\|\|\|\|\|\|\|\|\|\|\|\|\|\|\|\|\|\|\|\|\|\|\|\|\| \| \|\| \|\|\|\|\|\|\|\| \|\| | |
| SBJCT: | 5287 | GAGAAATCCATCACCGTTGACATTGAGAACTCCAACCGTGATAACGATGTCACTGTGATT | 5346 |
| QUERY: | 5398 | ACCAACCTCTCTTCAGTAGAGGCCTCCTACACAGTGGTACAAGATCAAGTTCGGAACAGC | 5457 |
| | | \|\|\|\|\|\|\|\|\|\|\|\|\|\|\|\|\| \|\|\|\|\|\|\|\|\|\|\|\|\|\| \|\|\|\|\|\|\|\|\|\|\|\|\|\|\|\|\| \|\|\|\|\|\|\|\|\| | |
| SBJCT: | 5347 | ACCAACCTCTCTTCAGTGGAGGCCTCCTACACCGTGGTACAAGATCAAGTGCGGAACAGC | 5406 |
| QUERY: | 5458 | TACCAGCTCTGTAATAATGGTACCCTGAGGGTGATGTATGCTAATGGGATGGGTATCAGC | 5517 |
| | | \|\|\|\|\|\|\|\|\|\|\| \| \|\| \|\| \|\|\|\|\|\| \| \|\| \|\|\|\|\| \|\| \|\| \|\| \|\|\|\|\| \|\|\|\|\| | |
| SBJCT: | 5407 | TACCAGCTCTGCAGCAACGGGACCCTGCGCGTCATGTACGCCAACGGCATGGGCGTCAGC | 5466 |
| QUERY: | 5518 | TTCCACAGCGAGCCCCATGTCCTAGCGGGCACCATCACCCCCACCATTGGACGCTGCAAC | 5577 |
| | | \|\|\|\|\|\|\|\|\|\|\|\|\|\|\|\|\| \|\|\|\|\| \|\| \|\|\|\|\|\| \|\|\|\|\|\|\|\|\|\|\|\|\| \|\| \|\|\|\|\| \|\|\| | |
| SBJCT: | 5467 | TTCCACAGCGAGCCCCACGTCCTCGCAGGCACCCTCACCCCCACCATCGGGCGCTGTAAC | 5526 |
| QUERY: | 5578 | ATCTCCCTGCCTATGGAGAATGGCTTAAACTCCATTGAGTGGCGCCTAAGAAAGGAACAG | 5637 |
| | | \|\|\|\|\|\|\|\|\|\|\| \|\|\|\|\|\|\|\| \|\|\| \| \|\|\|\|\|\|\|\| \|\|\|\|\|\|\|\|\|\|\| \|\| \|\|\|\|\|\|\|\|\| | |
| SBJCT: | 5527 | ATCTCCCTGCCCATGGAGAACGGCCTGAACTCCATCGAGTGGCGCCTGAGGAAGGAACAG | 5586 |
| QUERY: | 5638 | ATTAAAGGCAAAGTCACCATCTTTGGCAGGAAGCTCCGGGTCCATGGAAGAAATCTCTTG | 5697 |
| | | \|\|\|\|\|\|\|\|\|\|\|\|\|\|\|\|\|\|\|\|\|\|\|\|\|\| \|\|\|\|\|\|\|\| \|\|\|\|\|\|\|\| \|\|\|\|\| \|\| \|\|\| \|\| | |
| SBJCT: | 5587 | ATTAAAGGCAAAGTCACCATCTTTGGGAGGAAGCTTCGGGTCCACGGAAGGAACCTCCTG | 5646 |
| QUERY: | 5698 | TCCATTGACTATGATCGAAATATTCGGACTGAAAAGATCTATGATGACCACCGGAAGTTC | 5757 |
| | | \|\|\|\|\|\|\|\| \|\|\|\|\| \|\|\|\|\|\|\|\| \|\| \|\|\|\|\| \|\|\|\|\|\|\|\|\|\|\| \|\|\|\|\|\|\|\|\|\|\|\|\|\|\| | |
| SBJCT: | 5647 | TCCATTGATTATGACCGAAATATCCGCACTGAGAAGATCTATGACGACCACCGGAAGTTC | 5706 |
| QUERY: | 5758 | ACCCTGAGGATCATTTATGACCAGGTGGGCCGCCCCTTCCTCTGGCTGCCCAGCAGCGGG | 5817 |
| | | \|\|\|\|\|\|\|\|\|\|\|\|\|\|\|\|\|\|\|\|\|\|\|\|\|\|\|\|\|\|\|\|\|\|\|\|\|\|\|\|\| \|\|\|\|\| \|\|\|\|\|\|\|\| \|\| | |
| SBJCT: | 5707 | ACCCTGAGGATCATTTATGACCAGGTGGGCCGCCCCTTCCTGTGGCTCCCCAGCAGTGGA | 5766 |
| QUERY: | 5818 | CTGGCAGCTGTCAACGTGTCATACTTCTTCAATGGGCGCCTGGCTGGGCTTCAGCGTGGG | 5877 |
| | | \|\|\|\|\| \|\| \|\|\|\|\| \|\| \|\| \|\|\|\|\|\|\|\|\|\|\| \|\|\|\|\|\|\|\|\|\|\| \|\| \|\| \|\|\|\|\| \|\|\| | |
| SBJCT: | 5767 | CTGGCGGCCGTCAATGTCTCCTACTTCTTCAACGGGCGCCTGGCCGGCCTCCAGCGCGGG | 5826 |
| QUERY: | 5878 | GCCATGAGCGAGAGGACAGACATCGACAAGCAAGGCCGCATCGTGTCCCGCATGTTCGCT | 5937 |
| | | \|\|\|\|\|\|\|\|\|\|\|\|\|\|\|\|\|\|\|\|\|\|\| \|\|\|\|\|\|\|\|\|\|\|\|\|\| \|\| \|\|\|\|\|\|\|\| \|\|\|\|\|\|\|\| | |
| SBJCT: | 5827 | GCCATGAGCGAGAGGACAGACATTGACAAGCAAGGCCGGATTGTGTCCCGAATGTTCGCC | 5886 |
| QUERY: | 5938 | GACGGGAAAGTGTGGAGCTACTCCTACCTTGACAAGTCCATGGTCCTCCTGCTTCAGAGC | 5997 |
| | | \|\|\|\|\|\|\|\|\|\|\| \|\|\|\|\|\|\|\| \|\|\|\|\|\|\|\|\|\|\|\|\|\|\|\|\|\|\|\|\|\|\|\|\|\|\|\|\|\|\|\| \|\|\|\|\|\| | |
| SBJCT: | 5887 | GACGGGAAAGTCTGGAGCTATTCCTACCTTGACAAGTCCATGGTCCTCCTGCTGCAGAGC | 5946 |
| QUERY: | 5998 | CAACGTCAGTATATATTTGAGTATGACTCCTCTGACCGCCTCCTTGCCGTCACCATGCCC | 6057 |
| | | \|\| \|\|\|\|\|\|\|\| \|\|\|\|\|\|\|\| \|\|\|\|\|\|\|\|\|\|\|\|\|\|\|\|\|\|\|\|\|\| \|\| \|\|\|\|\|\|\|\|\|\|\|\| | |
| SBJCT: | 5924 | CAGCGTCAGTACATATTTGAATATGACTCCTCTGACCGCCTCCACGCAGTCACCATGCCC | 5983 |
| QUERY: | 6058 | AGCGTGGCCCGGCACAGCATGTCCACACACACCTCCATCGGCTACATCCGTAATATTTAC | 6117 |
| | | \|\| \|\| \|\|\|\|\|\|\|\|\|\|\|\|\|\|\|\|\|\|\|\| \|\|\|\|\|\|\|\|\|\|\| \|\|\|\|\|\|\|\|\|\|\| \|\| \|\|\|\|\|\| | |
| SBJCT: | 6007 | AGTGTCGCCCGGCACAGCATGTCCACGCACACCTCCATTGGCTACATCCGGAACATTTAC | 6066 |
| QUERY: | 6118 | AACCCGCCTGAAAGCAATGCTTCGGTCATCTTTGACTACAGTGATGACGGCCGCATCCTG | 6177 |
| | | \|\|\|\|\| \|\| \|\|\|\|\|\|\|\| \|\| \|\|\|\|\|\|\|\|\|\|\|\|\|\|\|\|\|\|\|\|\|\|\|\|\|\|\|\|\|\|\|\|\|\|\|\|\|\|\| | |
| SBJCT: | 6067 | AACCCACCGGAAAGCAACGCCTCGGTCATCTTTGACTACAGTGATGACGGCCGCATCCTG | 6126 |
| QUERY: | 6178 | AAGACCTCCTTTTTGGGCACCGGACGCCAGGTGTTCTACAAGTATGGGAAACTCTCCAAG | 6237 |
| | | \|\|\|\|\| \|\| \|\| \|\|\|\|\|\|\|\|\|\| \|\|\|\|\|\|\|\|\|\|\|\|\|\| \|\|\|\|\| \|\| \|\|\|\|\| \|\|\|\|\|\| | |
| SBJCT: | 6127 | AAGACGTCTTTCCTGGGCACCGGGCGCCAGGTGTTCTATAAGTACGGAAAACTCTCCAAG | 6186 |
| QUERY: | 6238 | TTATCAGAGATTGTCTACGACAGTACCGCCGTCACCTTCGGGTATGACGAGACCACTGGT | 6297 |
| | | \|\|\|\|\| \|\|\|\|\| \|\|\|\|\|\|\|\|\|\|\| \|\| \|\|\|\|\|\|\|\|\|\|\|\|\|\| \|\|\|\|\|\|\|\|\|\|\|\|\|\|\|\|\|\| | |
| SBJCT: | 6187 | TTATCGGAGATCGTCTACGACAGCACTGCCGTCACCTTCGGCTATGACGAGACCACTGGC | 6246 |
| QUERY: | 6298 | GTCTTGAAGATGGTCAACCTCCAAAGTGGGGGCTTCTCCTGCACCATCAGGTACCGGAAG | 6357 |
| | | \|\|\| \|\|\|\|\|\|\|\|\|\| \|\| \|\|\|\|\|\|\|\| \|\|\|\|\|\|\|\|\|\|\|\|\|\| \|\|\|\|\|\|\|\|\|\|\|\|\|\| \|\|\| | |
| SBJCT: | 6247 | GTCCTGAAGATGGTGAATCTCCAAAGCGGGGGCTTCTCCTGTACCATCAGGTACCGAAAG | 6306 |
| QUERY: | 6358 | ATTGGCCCCCTGGTGGACAAGCAGATCTACAGGTTCTCCGAGGAAGGCATGGTCAATGCC | 6417 |
| | | \| \|\| \|\|\|\|\| \|\|\|\|\|\|\|\|\|\|\|\|\|\| \|\|\|\|\|\|\|\|\|\|\| \|\|\|\|\|\|\|\|\|\|\|\| \|\|\|\| \|\|\| | |
| SBJCT: | 6307 | GTCGGGCCCCTCGTGGACAAGCAGATTTACAGGTTCTCTGAGGAAGGCATGATCAACGCC | 6366 |
| QUERY: | 6418 | AGGTTTGACTACACCTATCATGACAACAGCTTCCGCATCGCAAGCATCAAGCCCGTCATA | 6477 |
| | | \|\|\|\|\| \|\| \|\|\|\|\|\|\|\| \|\| \|\|\|\|\|\|\|\|\|\|\|\|\|\|\|\|\|\|\|\| \|\|\|\|\|\|\|\|\|\|\|\|\|\|\|\|\| | |
| SBJCT: | 6367 | AGGTTCGATTACACCTACCACGACAACAGCTTCCGCATCGCCAGCATCAAGCCCGTCATC | 6426 |
| QUERY: | 6478 | AGTGAGACTCCCCTCCCCGTTGACCTCTACCGCTATGATGAGATTTCTGGCAAGGTGGAA | 6537 |
| | | \|\|\|\|\|\|\|\|\|\|\|\|\|\| \|\|\|\|\|\|\|\|\|\|\|\|\|\|\|\|\|\|\|\| \|\|\|\|\|\|\|\|\|\|\|\|\|\|\|\|\|\|\|\|\|\|\|\| | |
| SBJCT: | 6427 | AGTGAGACTCCCCTTCCCGTTGACCTCTACCGCTACGATGAGATTTCTGGCAAGGTGGAA | 6486 |
| QUERY: | 6538 | CACTTTGGTAAGTTTGGAGTCATCTATTATGACATCAACCAGATCATCACCACTGCCGTG | 6597 |
| | | \|\|\|\|\| \|\| \|\|\|\|\| \|\| \|\|\|\|\|\|\|\| \|\| \|\|\|\|\|\|\|\|\|\|\|\|\|\|\|\|\|\|\|\|\|\|\|\|\|\|\|\|\| | |
| SBJCT: | 6487 | CACTTCGGCAAGTTCGGGGTCATCTACTACGACATCAACCAGATCATCACCACTGCCGTC | 6546 |
| QUERY: | 6598 | ATGACCCTCAGCAAACACTTCGACACCCATGGGCGGATCAAGGAGGTCCAGTATGAGATG | 6657 |
| | | \|\|\|\|\| \|\|\|\|\|\|\|\| \|\|\|\|\| \|\|\|\|\|\|\|\|\|\|\|\|\|\| \|\|\|\|\|\|\|\| \|\| \|\|\|\|\|\|\|\|\|\|\|\| | |
| SBJCT: | 6547 | ATGACACTCAGCAAGCACTTTGACACCCATGGGCGCATCAAGGAAGTGCAGTATGAGATG | 6606 |
| QUERY: | 6658 | TTCCGGTCCCTCATGTACTGGATGACGGTGCAATATGACAGCATGGGCAGGGTGATCAAG | 6717 |
| | | \|\|\|\|\|\|\|\|\|\|\|\|\|\|\|\|\|\|\|\|\|\|\|\|\|\|\|\|\|\|\|\|\|\|\|\|\|\|\|\|\| \|\|\|\|\|\|\|\|\|\|\| \|\|\|\|\|\| | |
| SBJCT: | 6607 | TTCCGGTCCCTCATGTACTGGATGACGGTGCAATATGACAGTATGGGCAGGGTCATCAAG | 6666 |
| QUERY: | 6718 | AGGGAGCTAAAACTGGGGCCCTATGCCAATACCACGAAGTACACCTATGACTACGATGGG | 6777 |
| | | \|\|\|\|\| \|\| \|\|\|\|\|\|\|\|\|\|\|\|\|\|\|\|\|\|\|\| \|\|\|\|\| \|\|\|\|\|\|\|\|\|\|\|\|\|\|\|\|\|\|\|\| \|\|\| | |
| SBJCT: | 6667 | AGGGAACTGAAACTGGGGCCCTATGCCAACACCACAAAGTACACCTATGACTACGACGGG | 6726 |
| QUERY: | 6778 | GACGGGCAGCTCCAGAGCGTGGCCGTCAATGACCGCCCGACCTGGCGCTACAGCTATGAC | 6837 |
| | | \|\|\|\|\| \|\|\|\|\|\|\|\|\|\|\| \|\|\|\|\|\|\|\|\|\|\|\|\|\|\|\|\| \|\| \|\|\|\|\|\|\|\| \|\| \|\|\|\|\|\|\|\|\| | |
| SBJCT: | 6727 | GACGGCCAGCTCCAGAGTGTGGCCGTCAATGACCGGCCTACCTGGCGTTATAGCTATGAC | 6786 |
| QUERY: | 6838 | CTTAATGGGAATCTCCACTTACTGAACCCAGGCAACAGTGTGCGCCTCATGCCCTTGCGC | 6897 |
| | | \|\| \|\|\|\|\|\|\|\| \|\| \|\|\| \| \|\| \|\|\|\|\|\|\|\| \|\|\|\|\|\|\| \|\|\|\|\|\|\|\|\|\|\| \|\| \|\|\| | |
| SBJCT: | 6787 | CTCAATGGGAACCTGCACCTGCTAAACCCAGGAAACAGTGCTCGCCTCATGCCGTTACGC | 6846 |
| QUERY: | 6898 | TATGACCTCCGGGATCGGATAACCAGACTCGGGGATGTGCAGTACAAAATTGACGACGAT | 6957 |
| | | \|\|\|\|\|\|\|\|\|\|\| \|\| \|\|\|\|\|\|\|\|\|\|\| \|\| \|\|\|\|\| \|\|\|\|\|\|\|\|\|\|\|\|\|\| \|\| \|\| \|\|\| | |
| SBJCT: | 6847 | TATGACCTCCGTGACCGGATAACCAGGCTAGGGGACGTGCAGTACAAAATCGATGATGAT | 6906 |
| QUERY: | 6958 | GGCTATCTGTGCCAGAGAGGGTCTGACATCTTCGAATACAATTCCAAGGGCCTCCTAACA | 7017 |
| | | \|\|\|\|\|\| \| \|\|\|\|\|\|\|\|\|\|\| \|\|\|\|\|\|\|\|\|\|\| \|\|\|\|\|\|\|\| \|\|\|\|\|\|\|\|\|\|\| \|\|\|\|\| | |
| SBJCT: | 6907 | GGCTATTTATGCCAGAGAGGATCTGACATCTTTGAATACAACTCCAAGGGCCTTCTAACG | 6966 |
| QUERY: | 7018 | AGAGCCTACAACAAGGCCAGCGGGTGGAGTGTCCAGTACCGCTATGATGGCGTAGGACGG | 7077 |
| | | \|\|\|\|\| \|\|\|\|\|\|\|\|\|\|\|\|\|\|\|\|\|\|\|\|\|\|\|\|\|\| \|\|\|\|\|\|\|\|\|\|\|\|\|\|\|\|\|\|\|\| \| \|\| | |
| SBJCT: | 6967 | AGAGCGTACAACAAGGCCAGCGGGTGGAGTGTGCAGTACCGCTATGATGGCGTGAGCCGC | 7026 |
| QUERY: | 7078 | CGGGCTTCCTACAAGACCAACCTGGGCCACCACCTGCAGTACTTCTACTCTGACCTCCAC | 7137 |
| | | \|\|\|\|\|\|\|\|\|\|\|\|\|\|\|\|\|\|\|\|\|\|\|\|\|\|\|\|\|\|\|\|\|\|\| \|\|\|\|\|\|\|\|\|\|\| \|\| \|\|\|\|\|\|\|\|\| | |
| SBJCT: | 7027 | CGGGCTTCCTACAAGACCAACCTGGGCCACCACCTACAGTACTTCTATTCCGACCTCCAC | 7086 |
| QUERY: | 7138 | AACCCGACGCGCATCACCCATGTCTACAATCACTCCAACTCGGAGATTACCTCACTGTAC | 7197 |
| | | \|\|\|\| \|\| \|\| \|\|\|\|\|\|\|\|\|\|\| \|\|\|\| \|\|\|\|\|\|\|\|\|\|\|\| \|\|\|\|\| \|\|\|\|\|\|\|\| \|\|\| | |
| SBJCT: | 7087 | CACCCCACACGTATCACCCATGTTTACAACCACTCCAACTCTGAGATCACCTCACTCTAC | 7146 |
| QUERY: | 7198 | TACGACCTCCAGGGCCACCTCTTTGCCATGGAGAGCAGCAGTGGGGAGGAGTACTATGTT | 7257 |
| | | \|\| \|\|\|\|\|\|\|\|\|\|\|\|\|\|\|\|\|\|\|\|\|\|\|\|\|\|\|\|\|\|\|\|\|\|\| \|\|\|\|\|\|\|\| \|\|\|\|\|\|\|\|\|\|\| | |
| SBJCT: | 7147 | TATGACCTCCAGGGCCACCTCTTTGCCATGGAGAGCAGTAGTGGGGAAGAGTACTATGTT | 7206 |
| QUERY: | 7258 | GCCTCTGATAACACAGGGACTCCTCTGGCTGTGTTCAGCATCAACGGCCTCATGATCAAA | 7317 |
| | | \|\|\|\|\| \|\|\|\|\|\|\|\| \|\|\|\|\|\|\|\|\|\|\|\|\|\|\|\|\| \|\| \|\| \|\|\|\|\| \|\|\|\|\|\|\|\|\|\|\|\|\|\| | |
| SBJCT: | 7207 | GCCTCAGATAACACCGGGACTCCTCTGGCTGTTTTTAGTATCAATGGCCTCATGATCAAG | 7266 |
| QUERY: | 7318 | CAGCTGCAGTACACGGCCTATGGGGAGATTTATTATGACTCCAACCCCGACTTCCAGATG | 7377 |
| | | \|\| \|\| \|\| \|\|\|\|\| \|\|\|\|\|\|\|\|\|\|\|\|\|\|\|\|\| \|\|\|\|\|\|\|\|\|\|\| \|\| \|\|\|\|\| \|\|\|\|\|\| | |
| SBJCT: | 7267 | CAACTCCAATACACAGCCTATGGGGAGATTTACTATGACTCCAATCCAGACTTTCAGATG | 7326 |
| QUERY: | 7378 | GTCATTGGCTTCCATGGGGGACTCTATGACCCCCTGACCAAGCTGGTCCACTTCACTCAG | 7437 |
| | | \|\|\|\|\| \|\|\|\|\|\|\|\| \|\| \|\| \|\|\|\|\| \|\|\|\|\|\|\|\| \|\|\|\|\|\|\|\| \|\| \|\|\|\|\| \|\| \|\|\| | |
| SBJCT: | 7327 | GTCATCGGCTTCCACGGAGGCCTCTACGACCCCCTCACCAAGCTCGTTCACTTTACGCAG | 7386 |
| QUERY: | 7438 | CGTGATTATGATGTGCTGGCAGGACGATGGACCTCCCCAGACTATACCATGTGGAAAAAC | 7497 |
| | | \|\|\|\|\|\|\|\|\|\|\| \|\|\|\|\|\|\|\|\|\|\|\|\|\| \|\|\|\|\| \|\|\|\|\| \|\|\|\|\| \|\|\|\|\|\|\|\|\|\|\| \|\| | |
| SBJCT: | 7387 | CGTGATTATGACGTGCTGGCAGGACCGTGGACGTCCCCCGACTACACCATGTGGAGGAAT | 7446 |
| QUERY: | 7498 | GTGGGCAAGGAGCCGGCCCCCTTTAACCTGTATATGTTCAAGAGCAACAATCCTCTCAGC | 7557 |
| | | \|\|\|\|\|\|\|\|\|\|\|\|\|\| \|\|\|\|\|\|\|\| \|\|\|\|\|\|\|\| \|\|\|\|\|\|\|\|\|\| \|\|\|\|\|\|\|\|\| \|\|\|\|\| | |
| SBJCT: | 7447 | GTGGGCAAGGAGCCAGCCCCCTTCAACCTGTACATGTTCAAGAACAACAATCCACTGAGC | 7506 |
| QUERY: | 7558 | AGTGAGCTAGATTTGAAGAACTACGTGACAGATGTGAAAAGCTGGCTTGTGATGTTTGGA | 7617 |
| | | \| \|\|\|\|\|\| \|\|\|\|\| \|\|\|\|\|\|\|\|\|\|\|\|\|\|\|\|\| \|\|\|\|\| \|\|\|\|\|\|\|\| \|\|\|\|\|\|\|\|\|\|\|\| | |
| SBJCT: | 7507 | AATGAGCTGGATTTAAAGAACTACGTGACAGACGTGAAGAGCTGGCTCGTGATGTTTGGA | 7566 |
| QUERY: | 7618 | TTTCAGCTTAGCAACATCATTCCTGGCTTCCCGAGAGCCAAAATGTATTTCGTGCCTCCT | 7677 |
| | | \|\|\|\|\|\|\|\| \|\|\|\|\|\|\|\|\|\|\|\|\|\|\|\|\| \|\|\|\|\| \|\|\|\|\|\|\|\|\|\|\|\|\|\|\|\|\| \|\|\|\|\|\|\|\| | |
| SBJCT: | 7567 | TTTCAGCTCAGCAACATCATTCCTGGATTCCCAAGAGCCAAAATGTATTTTGTGCCTCCC | 7626 |
| QUERY: | 7678 | CCCTATGAATTGTCAGAGAGTCAAGCAAGTGAGAATGGACAGCTCATTACAGGTGTCCAA | 7737 |
| | | \|\|\|\|\|\|\|\|\| \|\|\|\|\|\|\|\|\|\| \|\|\|\|\|\|\|\|\|\|\|\|\|\|\|\|\|\|\|\|\|\|\|\|\|\|\|\|\|\|\|\|\|\|\|\|\| | |
| SBJCT: | 7627 | CCCTATGAACTGTCAGAGAGCCAAGCAAGTGAGAATGGACAGCTCATTACAGGTGTCCAG | 7686 |
| QUERY: | 7738 | CAGACAACAGAGAGACATAACCAGGCCTTCATGGCTCTGGAAGGACAGGTCATTACTAAA | 7797 |
| | | \|\|\|\|\|\|\|\|\|\|\|\|\|\| \|\|\|\|\|\|\|\|\|\|\|\|\|\| \|\|\|\|\|\|\| \|\|\|\|\|\|\|\|\|\|\|\|\|\| \|\|\|\|\| | |
| SBJCT: | 7687 | CAGACAACAGAGAGGCATAACCAGGCCTTTCTGGCTCTAGAAGGACAGGTCATCTCTAAA | 7746 |
| QUERY: | 7798 | AAGCTCCACGCCAGCATCCGAGAGAAAGCAGGTCACTGGTTTGCCACCACCACGCCCATC | 7857 |
| | | \|\|\|\|\|\|\|\| \|\| \|\|\|\|\|\|\|\|\|\|\|\|\|\|\|\|\|\|\|\| \|\|\|\|\|\|\|\|\|\| \|\| \|\|\|\|\|\|\|\|\|\|\|\| | |
| SBJCT: | 7747 | AAGCTCCATGCAGGCATCCGAGAGAAAGCAGGCCACTGGTTTGCTACGACCACGCCCATC | 7806 |
| QUERY: | 7858 | ATTGGCAAAGGCATCATGTTTGCCATCAAAGAAGGGCGGGTGACCACGGGCGTGTCCAGC | 7917 |
| | | \|\| \|\|\|\|\|\|\|\|\|\|\|\|\|\|\|\|\| \|\|\|\|\|\|\|\|\|\|\|\|\|\|\|\|\|\|\|\|\|\|\|\|\|\| \|\|\|\|\|\|\|\| \|\|\| | |
| SBJCT: | 7807 | ATCGGCAAAGGCATCATGTTCGCCATCAAAGAAGGGCGGGTGACCACAGGCGTGTCTAGC | 7866 |
| QUERY: | 7918 | ATCGCCAGCGAAGATAGCCGCAAGGTGGCATCTGTGCTGAACAACGCCTACTACCTGGAC | 7977 |
| | | \|\|\|\|\|\|\|\| \|\| \|\| \|\|\|\|\|\|\|\|\|\|\| \|\|\|\|\| \|\|\| \|\|\|\|\|\|\|\|\|\|\|\|\|\|\|\|\| \|\|\|\|\| | |
| SBJCT: | 7867 | ATCGCCAGTGAGGACAGCCGCAAGGTAGCATCCGTGTTGAACAACGCCTACTACTTGGAC | 7926 |
| QUERY: | 7978 | AAGATGCACTACAGCATCGAGGGCAAGGACACCCACTACTTTGTGAAGATTGGCTCAGCC | 8037 |
| | | \|\|\|\|\|\|\|\|\|\|\|\|\|\|\|\|\|\|\|\|\|\|\|\|\|\|\|\|\|\|\|\| \|\|\|\|\|\|\|\| \|\|\|\|\|\|\|\| \|\| \|\|\|\| | |
| SBJCT: | 7927 | AAGATGCACTACAGCATCGAGGGCAAGGACACACACTACTTCGTGAAGATCGGTGCAGCG | 7986 |
| QUERY: | 8038 | GATGGCGACCTGGTCACACTAGGCACCACCATCGGCCGCAAGGTGCTAGAGAGCGGGGTG | 8097 |
| | | \|\| \|\| \|\|\|\|\|\|\|\| \|\| \|\| \|\| \|\|\|\|\|\|\|\| \|\| \|\|\|\|\|\|\|\|\|\|\| \|\|\|\|\|\|\|\|\|\|\|\| | |
| SBJCT: | 7987 | GACGGTGACCTGGTTACGCTGGGGACCACCATTGGGCGCAAGGTGCTGGAGAGCGGGGTG | 8046 |
| QUERY: | 8098 | AACGTGACCGTGTCCCAGCCCACGCTGCTGGTCAACGGCAGGACTCGAAGGTTCACGAAC | 8157 |
| | | \|\|\|\|\|\|\|\|\|\|\|\|\|\| \|\|\|\|\|\|\|\|\|\|\|\|\|\|\|\|\| \|\|\|\|\|\|\|\|\|\|\|\|\|\|\|\|\|\|\|\|\|\|\| \|\|\| | |
| SBJCT: | 8047 | AACGTGACCGTGTCACAGCCCACGCTGCTGGTGAACGGCAGGACTCGAAGGTTCACCAAC | 8106 |
| QUERY: | 8158 | ATTGAGTTCCAGTACTCCACGCTGCTGCTCAGCATCCGCTATGGCCTCACCCCCGACACC | 8217 |
| | | \|\|\|\|\| \|\|\|\|\|\|\|\|\|\|\|\|\|\|\|\|\|\|\|\|\|\|\|\|\|\|\|\|\| \|\|\|\|\| \|\|\|\|\|\|\|\|\|\|\|\|\|\|\|\|\| | |
| SBJCT: | 8107 | ATTGAATTCCAGTACTCCACGCTGCTGCTCAGCATACGCTACGGCCTCACCCCCGACACA | 8166 |
| QUERY: | 8218 | CTGGACGAAGAGAAGGCCCGCGTCCTGGACCAGGCGAGACAGAGGGCCCTGGGCACGGCC | 8277 |
| | | \|\|\|\|\| \|\|\|\|\|\|\|\|\|\|\|\|\|\|\|\|\|\|\|\|\|\|\|\|\|\| \|\|\| \|\|\|\|\|\|\|\|\|\|\|\|\|\|\|\| \|\| \|\|\| | |
| SBJCT: | 8167 | CTGGATGAAGAGAAGGCCCGCGTCCTGGACCAAGCGCGACAGAGGGCCCTGGGTACTGCC | 8226 |
| QUERY: | 8278 | TGGGCCAAGGAGCAGCAGAAAGCCAGGGACGGGAGAGAGGGGAGCCGCCTGTGGACTGAG | 8337 |
| | | \|\|\|\|\|\|\|\|\|\|\|\|\|\|\|\|\|\|\|\|\|\|\|\|\|\|\|\|\|\|\|\|\|\|\|\|\|\|\|\|\| \|\|\|\|\| \|\|\|\|\|\|\|\| \|\|\| | |
| SBJCT: | 8227 | TGGGCCAAGGAGCAGCAGAAAGCCAGGGACGGGAGAGAGGGCAGCCGTCTGTGGACGGAG | 8286 |
| QUERY: | 8338 | GGCGAGAAGCAGCAGCTTCTGAGCACCGGGCGCGTGCAAGGGTACGAGGGATATTACGTG | 8397 |
| | | \|\|\|\|\|\|\|\|\|\|\|\|\|\| \|\| \|\|\|\|\|\|\|\| \|\| \|\| \|\|\|\|\|\|\|\| \|\| \|\|\|\|\| \|\|\|\|\|\|\|\| | |
| SBJCT: | 8287 | GGCGAGAAGCAGCAACTCCTGAGCACGGGACGGGTGCAAGGTTATGAGGGCTATTACGTG | 8346 |
| QUERY: | 8398 | CTTCCCGTGGAGCAATACCCAGAGCTTGCAGACAGTAGCAGCAACATCCAGTTTTTAAGA | 8457 |
| | | \|\|\|\|\| \|\|\|\|\| \|\| \|\|\|\|\|\|\|\|\|\|\| \|\|\|\|\|\|\|\|\|\|\|\|\|\|\|\|\|\|\|\|\|\|\|\|\|\| \|\|\|\|\|\| | |
| SBJCT: | 8347 | CTTCCGGTGGAACAGTACCCAGAGCTGGCAGACAGTAGCAGCAACATCCAGTTCTTAAGA | 8406 |
| QUERY: | 8458 | CAGAATGAGATGGGAAAGAGGTAACAAAATAATCTGCTGCCATTCCTTGTCTGAATGGCT | 8517 |
| | | \|\|\|\|\|\|\|\|\|\|\|\|\|\|\|\|\|\|\|\|\|\|\|\|\|\|\|\|\|\|\|\| \|\|\|\|\|\|\|\|\| \|\|\| \|\|\|\| \|\|\|\|\| | |
| SBJCT: | 8407 | CAGAATGAGATGGGAAAGAGGTAACAAAATAACCTGCTGCCACCTCTTCTCTGGGTGGCT | 8466 |
| QUERY: | 8518 | CAGCAGGAGTAACTGTTATCTCCTCTCCTAAGGAGATGAAGACCTAACAGGGGCACTGCG | 8577 |
| | | \|\|\|\|\|\|\|\|\| \|\|\|\|\|\| \| \|\|\|\|\|\|\|\|\|\|\|\|\|\|\|\|\| \|\|\|\|\|\|\|\|\|\|\|\|\|\|\|\|\|\|\|\|\| \| | |
| SBJCT: | 8467 | CAGCAGGAGCAACTGTGACCTCCTCTCCTAAGGAGACGAAGACCTAACGGGGGCACTGAG | 8526 |
| QUERY: | 8578 | GCTGGGCTGCTTTAGGAGACCAAGTGGCAAGAAAGCTCACATTTTTTGAGTTCAAATGCT | 8637 |
| | | \|\| \|\|\|\|\|\|\|\|\|\|\|\|\|\| \|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 8527 | GCCGGGCTGCTTTAGGACCCCAAGTGGCAAGAAAGCTCACATTTTTTGAGTTCAAATGCT | 8586 |
| QUERY: | 8638 | ACTGTCCAAGCGAGAAGTCCCTCATCCTGAAGTAGACTAAAGCCCGGCTGAAAATTCCGA | 8685 |
| | | \|\|\|\|\|\|\|\|\|\|\|\| \|\|\|\|\|\|\|\|\|\|\|\|\|\|\|\|\|\|\|\|\|\|\|\|\| \|\|\| \|\|\|\| \|\|\|\|\|\| \|\| | |
| SBJCT: | 8587 | ACTGTCCAAGCGCAAAGTCCCTCATCCTGAAGTAGACTAGAGCTCGGCCACAAATTCTGA | 8610 |
| QUERY: | 8698 | GGAAAACAAAAC 8709 | |
| | | \|\|\|\|\|\|\|\|\|\|\|\| | |
| SBJCT: | 8647 | GGAAAACAAAAC 8658 | |

SCORE = 1459 BITS (736), EXPECT = 0.0
IDENTITIES = 1081/1196 (90%)
STRAND = PLUS/PLUS

| | | | |
|---|---|---|---|
| QUERY: | 270 | ATCTGGAATAATGGATGTAAAGGACCGGCGACACCGCTCTTTGACCAGAGGACGCTGTGG | 329 |
| | | \|\|\|\|\| \|\|\|\|\|\|\|\|\|\|\|\| \|\|\|\|\| \|\|\|\|\|\|\|\| \|\|\|\|\|\|\|\|\|\|\|\|\|\| \|\|\|\|\| \|\|\|\|\| | |
| SBJCT: | 123 | ATCTGCAATAATGGATGTGAAGGATCGGCGACATCGCTCTTTGACCAGGGGACGGTGTGG | 182 |
| QUERY: | 330 | CAAAGAGTGTCGCTACACAAGCTCCTCTCTGGACAGTGAGGACTGCCGGGTGCCCACACA | 389 |
| | | \|\|\| \|\|\|\|\|\|\|\|\|\|\|\|\|\| \|\|\|\|\|\|\|\|\|\|\|\|\|\|\|\|\|\|\|\|\|\|\|\|\|\|\|\|\| \|\|\|\|\|\|\|\| \|\| | |
| SBJCT: | 183 | CAAGGAGTGTCGCTACACCAGCTCCTCTCTGGACAGTGAGGACTGCCGTGTGCCCACGCA | 242 |
| QUERY: | 390 | GAAATCCTACAGCTCCAGTGAGACTCTGAAGGCCTATGACCATGACAGCAGGATGCACTA | 449 |
| | | \|\|\| \|\|\|\|\|\|\|\| \|\|\|\|\|\|\|\|\|\|\| \|\|\|\|\|\|\|\| \|\|\|\|\|\|\|\|\|\|\|\|\|\|\|\|\| \|\|\|\|\|\|\|\| | |
| SBJCT: | 243 | GAAGTCCTACAGTTCCAGTGAGACCCTGAAGGCTTATGACCATGACAGCAGAATGCACTA | 302 |
| QUERY: | 450 | TGGAAACCGAGTCACAGACCTCATCCACCGGGAGTCAGATGAGTTTCCTAGACAAGGAAC | 509 |
| | | \|\|\|\|\|\|\|\|\|\|\|\|\|\|\|\|\|\|\|\|\| \| \|\|\|\|\|\|\|\|\|\|\| \|\|\|\|\|\|\|\|\| \|\|\|\|\|\|\|\|\|\| \| | |
| SBJCT: | 303 | TGGAAACCGAGTCACAGACCTGGTGCACCGGGAGTCCGATGAGTTTTCTAGACAAGGGGC | 362 |
| QUERY: | 510 | CAACTTCACCCTTGCCGAACTGGGCATCTGTGAGCCCTCCCCACACCGAAGCGGCTACTG | 569 |
| | | \|\| \|\|\|\|\|\|\|\| \|\| \|\|\| \|\|\|\| \|\|\|\|\| \|\|\|\|\|\|\|\|\|\|\|\|\|\|\|\|\|\|\|\| \|\| \|\|\|\|\| | |
| SBJCT: | 363 | TAATTTCACCCTGGCAGAATTGGGAATCTGCGAGCCCTCCCCACACCGAAGTGGTTACTG | 422 |
| QUERY: | 570 | CTCCGACATGGGGATCCTTCACCAGGGCTACTCCCTTAGCACAGGGTCTGACGCCGACTC | 629 |
| | | \|\|\|\|\|\|\|\|\|\|\|\|\|\|\|\|\| \|\|\|\|\|\|\|\|\|\|\|\|\|\|\|\|\| \|\|\|\|\| \|\|\|\|\|\|\|\| \|\| \|\|\|\|\| | |
| SBJCT: | 423 | TTCCGACATGGGGATCCTCCACCAGGGCTACTCCCTGAGCACTGGGTCTGATGCGGACTC | 482 |
| QUERY: | 630 | CGACACCGAGGGAGGGATGTCTCCAGAACACGCCATCAGACTGTGGGGCAGAGGGATAAA | 689 |
| | | \|\|\|\|\|\|\|\|\|\|\|\|\|\|\|\|\|\|\|\|\|\|\|\|\|\|\|\|\| \|\|\|\|\|\|\|\|\|\|\|\|\|\|\|\|\| \|\|\|\|\|\|\|\|\|\| | |
| SBJCT: | 483 | GGACACCGAGGGAGGGATGTCTCCAGAACATGCCATCAGACTGTGGGGACGAGGGATAAA | 542 |
| QUERY: | 690 | ATCCAGGCGCAGTTCCGGCCTGTCCAGTCGTGAAAACTCGGCCCTTACCCTGACTGACTC | 749 |
| | | \|\|\| \|\|\|\|\|\|\|\| \|\| \|\|\| \|\|\|\|\|\|\| \|\| \|\| \|\|\|\|\| \|\|\|\|\|\|\|\| \|\|\|\|\|\|\|\| \|\| | |
| SBJCT: | 543 | ATCGAGGCGCAGCTCTGGCTTGTCCAGCCGCGAGAACTCAGCCCTTACTCTGACTGATTC | 602 |
| QUERY: | 750 | TGACAACGAAAACAAATCAGATGATGAGAACGGTCGTCCCATTCCACCTACATCCTCGCC | 809 |
| | | \|\|\|\|\|\| \|\|\|\|\| \|\|\|\|\| \|\|\|\|\| \|\| \|\| \|\|\|\|\| \|\|\|\|\|\|\|\|\|\|\|\|\|\|\|\|\|\|\|\|\| \| | |
| SBJCT: | 603 | TGACAATGAAAATAAATCGGATGACGACAATGGTCGACCCATTCCACCTACATCCTCGTC | 662 |
| QUERY: | 810 | TAGTCTCCTCCCATCTGCTCAGCTGCCTAGCTCCCATAATCCTCCACCAGTTAGCTGCCA | 869 |
| | | \|\|\| \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 663 | TAGCCTCCTCCCATCTGCTCAGCTGCCTAGCTCCCATAATCCTCCACCAGTTAGCTGCCA | 722 |
| QUERY: | 870 | GATGCCATTGCTAGACAGCAACACCTCCCATCAAATCATGGACACCAACCCTGATGAGGA | 929 |
| | | \|\|\|\|\|\|\|\|\|\|\|\|\|\|\|\|\|\|\|\|\|\|\|\|\|\|\|\|\|\|\|\|\| \|\|\|\|\|\|\|\|\|\|\|\|\|\|\|\|\| \|\|\|\|\|\|\|\| | |
| SBJCT: | 723 | GATGCCATTGCTAGACAGCAACACCTCCCATCAGATCATGGACACCAACCCCGATGAGGA | 782 |
| QUERY: | 930 | ATTCTCCCCCAATTCATACCTGCTCAGAGCATGCTCAGGGCCCCAGCAAGCCTCCAGCAG | 989 |
| | | \|\|\|\|\|\|\|\|\| \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| \|\| | |
| SBJCT: | 783 | ATTCTCCCCTAATTCATACCTGCTCAGAGCATGCTCAGGGCCCCAGCAAGCCTCCAGTAG | 842 |
| QUERY: | 990 | TGGCCCTCCGAACCACCACAGCCAGTCGACTCTGAGGCCCCCTCTCCCACCCCCTCACAA | 1049 |
| | | \|\|\|\|\|\|\|\|\|\|\|\|\|\|\|\|\|\|\|\|\|\|\|\|\|\|\| \|\| \|\|\|\|\|\|\|\|\|\|\|\|\|\| \|\|\|\|\| \|\|\|\|\| \|\| | |
| SBJCT: | 843 | TGGCCCTCCGAACCACCACAGCCAGTCAACGCTGAGGCCCCCTCTGCCACCTCCTCATAA | 902 |
| QUERY: | 1050 | CCACACGCTGTCCCATCACCACTCGTCCGCCAACTCCCTCAACAGGAACTCACTGACCAA | 1109 |
| | | \|\|\|\|\|\| \|\|\|\|\|\|\|\| \|\|\|\|\|\|\|\| \|\| \|\|\|\|\|\|\|\|\|\|\|\|\|\|\|\|\| \|\|\|\|\|\|\|\|\|\|\|\|\|\| | |
| SBJCT: | 903 | CCACACCCTGTCCCACCACCACTCCTCTGCCAACTCCCTCAACAGAAACTCACTGACCAA | 962 |
| QUERY: | 1110 | TCGGCGGAGTCAGATCCACGCCCCGGCCCCAGCGCCCAATGACCTGGCCACCACACCAGA | 1169 |
| | | \|\|\|\|\|\|\|\|\|\|\|\| \|\|\|\|\|\|\|\|\|\|\| \|\| \|\| \|\| \|\|\|\|\|\|\|\|\|\|\|\|\|\|\|\|\|\|\|\| \|\| \|\| | |
| SBJCT: | 963 | TCGGCGGAGTCAAATCCACGCCCCAGCTCCTGCACCCAATGACCTGGCCACCACGCCAGA | 1022 |
| QUERY: | 1170 | GTCCGTTCAGCTTCAGGACAGCTGGGTGCTAAACAGCAACGTGCCACTGGAGACCCGGCA | 1229 |
| | | \|\|\|\|\|\|\|\|\|\|\|\| \|\|\|\|\|\|\|\|\|\|\|\|\|\|\|\|\| \|\|\|\|\| \|\|\|\|\|\|\|\| \|\|\|\|\|\|\|\| \|\|\|\|\| | |
| SBJCT: | 1023 | GTCCGTTCAGCTCCAGGACAGCTGGGTGCTGAACAGTAACGTGCCGCTGGAGACGCGGCA | 1082 |
| QUERY: | 1230 | CTTCCTCTTCAAGACCTCCTCGGGGAGCACACCCTTGTTCAGCAGCTCTTCCCCGGGATA | 1289 |
| | | \|\|\|\|\|\|\|\|\|\|\|\|\|\|\| \|\|\|\|\| \|\| \|\|\|\|\|\|\|\|\| \|\|\|\|\|\|\|\|\|\|\|\|\|\|\|\| \|\| \|\|\|\|\| | |
| SBJCT: | 1083 | CTTCCTCTTCAAGACGTCCTCCGGAAGCACACCCCTGTTCAGCAGCTCTTCTCCAGGATA | 1142 |
| QUERY: | 1290 | CCCTTTGACCTCAGGAACGGTTTACACGCCCCCGCCCCGCCTGCTGCCCAGGAATACTTT | 1349 |
| | | \|\|\| \|\|\|\|\|\|\|\|\|\|\| \|\| \|\|\|\|\| \|\| \|\| \|\| \|\|\|\|\|\|\|\|\|\|\|\|\|\| \|\|\|\|\|\|\| \|\| | |
| SBJCT: | 1143 | CCCCTTGACCTCAGGGACCGTTTATACACCACCACCCCGCCTGCTGCCACGGAATACATT | 1202 |
| QUERY: | 1350 | CTCCAGGAAGGCTTTCAAGCTGAAGAAGCCCTCCAAATACTGCAGCTGGAAATGTGCTGC | 1409 |
| | | \|\|\| \|\|\|\|\|\|\|\| \|\|\|\|\|\|\|\|\|\|\|\|\|\| \|\|\|\|\|\|\|\|\|\|\|\|\|\|\|\|\| \|\|\|\|\|\|\|\| \|\| \|\| | |
| SBJCT: | 1203 | CTCTAGGAAGGCCTTCAAGCTGAAGAAACCCTCCAAATACTGCAGTTGGAAATGCGCCGC | 1262 |

| | | |
|---|---|---|
| QUERY: | 1410 | CCTCTCCGCCATTGCCGCGGCCCTCCTCTTGGCTATTTTGCTGGCGTATTTCATAG 1465 |
| | | \|\|\| \|\| \|\|\|\|\|\|\|\|\|\|\| \|\|\|\|\|\|\|\| \|\|\|\|\| \|\|\|\|\|\|\|\|\|\| \|\|\|\|\|\|\|\|\|\| |
| SBJCT: | 1263 | CCTGTCTGCCATTGCCGCTGCCCTCCTTCTGGCCATTTTGCTGGCCTATTTCATAG 1318 |

SCORE = 1427 BITS (720), EXPECT = 0.0
IDENTITIES = 996/1088 (91%)
STRAND = PLUS/PLUS

| | | | |
|---|---|---|---|
| QUERY: | 1464 | AGTGCCCTGGTCGTTGAAAAACAGCAGCATAGACAGTGGTGAAGCAGAAGTTGGTCGGCG | 1523 |
| | | \|\|\|\|\|\|\|\|\|\|\|\|\|\|\|\|\|\|\|\|\|\|\|\|\|\|\|\|\|\|\|\|\|\|\|\| \|\| \|\| \|\|\|\|\|\|\|\| \|\|\|\|\| \|\| | |
| SBJCT: | 1440 | AGTGCCCTGGTCGTTGAAAAACAGCAGCATAGACAGCGGCGAGGCAGAAGTCGGTCGACG | 1499 |
| QUERY: | 1524 | GGTAACACAAGAAGTCCCACCAGGGGTGTTTTGGAGGTCACAAATTCACATCAGTCAGCC | 1583 |
| | | \|\|\| \|\|\|\|\| \|\|\|\|\|\|\|\|\|\|\|\|\|\|\|\|\|\|\|\|\|\|\|\|\|\|\|\|\| \|\| \|\|\|\|\|\|\|\|\|\|\|\|\|\|\|\|\| | |
| SBJCT: | 1500 | GGTGACACAGGAAGTCCCACCAGGGGTGTTTTGGAGGTCCCAGATTCACATCAGTCAGCC | 1559 |
| QUERY: | 1584 | CCAGTTCTTAAAGTTCAACATCTCCCTCGGGAAGGACGCTCTCTTTGGTGTTTACATAAG | 1643 |
| | | \|\|\|\|\|\|\|\|\|\|\|\|\|\|\|\|\|\|\|\|\|\|\|\|\|\| \|\|\|\|\|\|\|\| \|\| \|\|\|\|\| \|\| \|\| \|\|\|\|\|\|\|\| | |
| SBJCT: | 1560 | TCAGTTCTTAAAGTTCAACATCTCCCTGGGGAAGGATGCCCTCTTCGGCGTCTACATAAG | 1619 |
| QUERY: | 1644 | AAGAGGACTTCCACCATCTCATGCCCAGTATGACTTCATGGAACGTCTGGACGGGAAGGA | 1703 |
| | | \|\|\|\|\|\|\|\|\| \|\|\|\|\|\|\|\|\|\|\|\|\|\| \|\|\|\|\|\|\|\|\|\|\|\|\|\|\|\|\|\|\|\| \|\|\|\|\|\|\|\| \|\|\|\|\| | |
| SBJCT: | 1620 | AAGAGGACTGCCACCATCTCATGCACAGTATGACTTCATGGAACGCCTGGACGGAAAGGA | 1679 |
| QUERY: | 1704 | GAAGTGGAGTGTGGTTGAGTCTCCCAGGGAACGCCGGAGCATACAGACCTTGGTTCAGAA | 1763 |
| | | \|\|\|\|\|\|\|\|\|\|\|\|\|\|\| \|\|\|\|\| \|\|\|\|\|\|\|\|\|\|\|\|\|\|\|\|\|\|\|\| \|\|\|\|\|\| \|\|\|\| \|\|\|\|\| | |
| SBJCT: | 1680 | GAAGTGGAGTGTGGTCGAGTCACCCAGGGAACGCCGGAGCATCCAGACCCTGGTGCAGAA | 1739 |
| QUERY: | 1764 | TGAAGCCGTGTTTGTGCAGTACCTGGATGTGGGCCTGTGGCATCTGGCCTTCTACAATGA | 1823 |
| | | \|\| \|\| \|\|\|\|\| \|\|\|\|\|\|\|\|\| \|\|\|\|\|\|\|\|\|\|\|\|\|\|\|\|\|\|\| \|\| \|\|\|\|\|\|\|\|\|\|\|\|\|\| | |
| SBJCT: | 1740 | CGAGGCTGTGTTCGTGCAGTACTTGGATGTGGGCCTGTGGCACCTCGCCTTCTACAATGA | 1799 |
| QUERY: | 1824 | TGGAAAAGACAAAGAGATGGTTTCCTTCAATACTGTTGTCCTAGATTCAGTGCAGGACTG | 1883 |
| | | \|\| \|\| \|\|\|\|\| \|\|\|\|\|\|\|\| \|\|\|\|\|\|\|\|\|\|\| \|\|\|\|\|\| \|\|\|\|\|\|\|\|\|\|\|\|\|\|\|\|\|\|\| | |
| SBJCT: | 1800 | CGGCAAGGACAAGGAGATGGTCTCCTTCAATACGGTTGTCTTAGATTCAGTGCAGGACTG | 1859 |
| QUERY: | 1884 | TCCACGTAACTGCCATGGGAATGGTGAATGTGTGTCCGGGGTGTGTCACTGTTTCCCAGG | 1943 |
| | | \|\|\|\|\|\| \|\|\|\|\|\|\|\| \|\|\|\|\| \|\| \|\|\|\|\| \|\|\|\|\| \|\| \|\|\|\|\|\|\|\|\|\|\|\|\|\|\|\|\|\|\| | |
| SBJCT: | 1860 | TCCACGAAACTGCCACGGGAACGGCGAATGCGTGTCTGGACTGTGTCACTGTTTCCCAGG | 1919 |
| QUERY: | 1944 | ATTTCTAGGAGCAGACTGTGCTAAAGCTGCCTGCCCTGTCCTGTGCAGTGGGAATGGACA | 2003 |
| | | \|\|\| \|\|\|\|\| \|\|\|\|\|\|\|\| \|\|\|\|\|\|\|\|\|\|\|\|\|\|\|\|\|\|\|\| \|\|\|\|\|\|\|\|\|\|\|\|\|\|\|\|\|\|\|\| | |
| SBJCT: | 1920 | ATTCCTAGGTGCAGACTGCGCTAAAGCTGCCTGCCCTGTTCTGTGCAGTGGGAATGGACA | 1979 |
| QUERY: | 2004 | ATATTCTAAAGGGACGTGCCAGTGCTACAGCGGCTGGAAAGGTGCAGAGTGCGACGTGCC | 2063 |
| | | \|\|\|\|\| \|\|\|\|\|\|\|\| \|\|\|\|\|\|\|\|\|\|\|\|\|\| \|\|\|\|\|\|\|\|\|\|\| \|\|\|\|\| \|\|\|\|\| \|\|\|\|\| | |
| SBJCT: | 1980 | GTATTCCAAAGGGACATGCCAGTGCTACAGTGGCTGGAAAGGAGCAGAATGCGATGTGCC | 2039 |
| QUERY: | 2064 | CATGAATCAGTGCATCGATCCTTCCTGCGGGGGCCACGGCTCCTGCATTGATGGGAACTG | 2123 |
| | | \|\|\|\|\|\| \|\|\|\|\|\|\|\|\|\|\|\|\|\|\|\|\|\|\|\| \|\|\|\|\|\|\|\|\|\|\|\|\|\|\|\|\|\|\|\|\|\|\|\|\|\|\|\|\|\|\|\| | |
| SBJCT: | 2040 | CATGAACCAGTGCATCGATCCTTCCTGTGGGGGCCACGGCTCCTGCATTGATGGGAACTG | 2099 |
| QUERY: | 2124 | TGTCTGCTCTGCTGGCTACAAAGGCGAGCACTGTGAGGAAGTTGATTGCTTGGATCCCAC | 2183 |
| | | \|\| \|\| \| \|\|\|\|\|\|\|\|\|\|\| \|\|\|\|\|\|\|\|\|\|\| \|\| \|\|\|\|\| \|\|\|\|\|\|\|\|\|\|\|\|\|\| \|\| | |
| SBJCT: | 2100 | CGTGTGTGCAGCTGGCTACAAGGGCGAGCACTGCGAAGAAGTGGATTGCTTGGATCCAAC | 2159 |
| QUERY: | 2184 | CTGCTCCAGCCACGGAGTCTGTGTGAATGGAGAATGCCTGTGCAGCCCTGGCTGGGGTGG | 2243 |
| | | \|\|\|\|\|\|\|\|\|\|\|\| \|\| \|\|\|\|\|\|\|\|\|\|\| \|\|\|\|\| \|\| \|\| \|\|\|\|\|\|\|\| \|\|\|\|\|\|\|\| \|\| | |
| SBJCT: | 2160 | CTGCTCCAGCCATGGTGTCTGTGTGAACGGAGAGTGTCTATGCAGCCCCGGCTGGGGCGG | 2219 |
| QUERY: | 2244 | TCTGAACTGTGAGCTGGCGAGGGTCCAGTGCCCAGACCAGTGCAGTGGGCATGGCACGTA | 2303 |
| | | \|\| \|\|\|\|\| \|\|\|\|\|\|\|\|\|\|\|\|\|\|\|\|\|\|\|\|\|\|\|\|\|\|\|\|\|\|\|\| \|\|\|\|\|\|\|\|\|\|\|\|\|\| \|\| | |
| SBJCT: | 2220 | GCTCAACTGCGAGCTGGCGAGGGTCCAGTGCCCAGACCAGTGTAGTGGGCATGGCACTTA | 2279 |
| QUERY: | 2304 | CCTGCCTGACACGGGCCTCTGCAGCTGCGATCCCAACTGGATGGGTCCCGACTGCTCTGT | 2363 |
| | | \|\|\| \|\|\|\|\|\| \| \|\|\|\|\|\|\|\|\|\| \|\|\| \|\|\|\|\| \|\| \|\|\|\|\|\|\|\|\|\|\|\|\|\|\|\|\|\|\|\|\|\|\| | |
| SBJCT: | 2280 | CCTCCCTGACTCTGGCCTCTGCAACTGTGATCCGAATTGGATGGGTCCCGACTGCTCTGT | 2339 |
| QUERY: | 2364 | TGAAGTGTGCTCAGTAGACTGTGGCACTCACGGCGTCTGCATCGGGGGAGCCTGCCGCTG | 2423 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 2340 | TGAAGTGTGCTCAGTAGACTGTGGCACTCACGGCGTCTGCATCGGGGGAGCCTGCCGCTG | 2399 |
| QUERY: | 2424 | TGAAGAGGGCTGGACAGGCGCAGCGTGTGACCAGCGCGTGTGCCACCCCCGCTGCATTGA | 2483 |
| | | \|\|\|\|\|\|\|\|\|\|\|\|\|\|\|\|\|\|\|\|\| \|\| \|\|\|\|\|\|\|\|\|\|\|\|\|\|\|\|\|\|\|\|\|\|\|\|\|\|\|\|\|\|\|\|\|\|\| | |
| SBJCT: | 2400 | TGAAGAGGGCTGGACAGGCGCGGCTTGTGACCAGCGCGTGTGCCACCCCCGCTGCATTGA | 2459 |
| QUERY: | 2484 | GCACGGGACCTGTAAAGATGGCAAATGTGAATGCCGAGAGGGCTGGAATGGTGAACACTG | 2543 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 2460 | GCACGGGACCTGTAAAGATGGCAAATGTGAATGCCGAGAGGGCTGGAATGGTGAACACTG | 2519 |
| QUERY: | 2544 | CACCATTG 2551 | |
| | | \|\|\|\|\|\|\|\| | |
| SBJCT: | 2520 | CACCATTG 2527 | |

In this search it was also found that the FCTR3bcd and e nucleic acid had homology to six fragments of Gallus gallus partial mRNA for teneurin-2. It has 2780 of 3449 bases (80%) identical to bases 3386-6834, 1553 of 1862 bases (83%) identical to bases 1414-3275, 540 of 628 bases (85%) identical to bases 587-1214, 593 of 725 bases (81%) identical to bases 7084-7808, 429 of 515 bases (83%) identical to bases 7895-8409, and 397 of 475 bases (83%) identical to bases 20-494 of Gallus gallus partial mRNA for teneurin-2. (EMBL Acc: GGA278031) (Table 3O).

TABLE 30
BLASTN of FCTR3b,c,d, and e against Gallus gallus Teneurin-2 mRNA
(SEQ ID NO:67)

>GI|10241573|EMB|AJ279031.1|GGA279031 GALLUS GALLUS PARTIAL MRNA FOR
TENEURIN-2 (TEN2 GENE), LONG SPLICE
VARIANT
LENGTH = 8409
SCORE = 1532 BITS (773), EXPECT = 0.0
IDENTITIES = 2780/3449 (80%)
STRAND = PLUS/PLUS

| | | | |
|---|---|---|---|
| QUERY: | 3458 | TGATGGTGGCTGTCGAGGGGCATCTCTTCCAGAAGTCATTCCAGGCTTCTCCCAACCTGG | 3517 |
| | | \|\|\|\|\|\|\| \|\| \|\| \|\| \|\|\|\|\|\|\|\| \|\| \|\| \|\| \|\|\|\|\| \| \|\|\| \|\|\|\|\|\|\|\|\| \|\|\| | |
| SBJCT: | 3386 | TGATGGTAGCAGTAGAAGGGCATCTATTTCAAAAATCATTTCTGGCATCTCCCAACTTGG | 3445 |
| QUERY: | 3518 | CCTCCACCTTCATCTGGGACAAGACAGATGCGTATGGCCAAAGGGTGTATGGACTCTCAG | 3577 |
| | | \| \| \|\| \|\|\|\|\|\|\|\|\|\|\|\|\|\| \|\|\|\|\|\|\|\| \|\|\|\|\| \|\| \|\|\| \|\|\|\|\| \| \|\|\|\| | |
| SBJCT: | 3446 | CTTATACATTCATCTGGGACAAAACAGATGCATATGGTCAGAAGGTTTATGGGTTGTCAG | 3505 |
| QUERY: | 3578 | ATGCTGTTGTGTCTGTCGGGTTTGAATATGAGACCTGTCCCAGTCTAATTCTCTGGGAGA | 3637 |
| | | \|\|\|\|\|\|\| \|\| \|\|\|\|\| \|\| \|\|\|\|\|\|\|\|\|\|\|\|\|\| \|\|\|\|\|\|\|\|\| \| \|\|\|\|\| \|\|\|\|\|\|\| | |
| SBJCT: | 3506 | ATGCTGTAGTTTCTGTGGGTTTTGAATATGAGACTTGTCCCAGTTTGATTCTGTGGGAGA | 3565 |
| QUERY: | 3638 | AAAGGACAGCCCTCCTTCAGGGATTCGAGCTGGACCCCTCCAACCTCGGTGGCTGGTCCC | 3697 |
| | | \|\|\|\|\|\|\| \|\| \|\| \|\| \|\| \|\|\|\|\| \|\|\|\|\| \|\| \|\| \|\|\|\|\| \|\| \|\| \|\| \|\|\|\|\| | |
| SBJCT: | 3566 | AAAGGACTGCGCTGCTGCAAGGATTTGAGCTAGATCCTTCCAATCTAGGAGGATGGTCTT | 3625 |
| QUERY: | 3698 | TAGACAAACACCACATCCTCAATGTTAAAAGTGGAATCCTACACAAAGGCACTGGGGAAA | 3757 |
| | | \| \|\| \|\|\|\|\| \|\| \| \|\| \|\|\|\|\| \|\| \|\|\|\|\| \|\| \| \|\|\|\|\|\|\|\|\|\| \|\|\| \|\|\|\| | |
| SBJCT: | 3626 | TGGATAAACATCATGTACTGAATGTCAAGAGTGGTATATTGCACAAAGGCAATGGAGAAA | 3685 |
| QUERY: | 3758 | ACCAGTTCCTGACCCAGCAGCCTGCCATCATCACCAGCATCATGGGCAATGGTCGCCGCC | 3817 |
| | | \| \|\|\|\|\| \|\| \|\| \|\|\|\|\|\|\|\| \|\| \| \|\| \|\|\|\|\|\|\|\| \|\|\|\|\| \|\|\|\|\| \|\|\|\|\| | |
| SBJCT: | 3686 | ATCAGTTTCTAACTCAGCAGCCAGCTGTGATAACCAGCATTATGGGGAATGGGCGCCGAA | 3745 |
| QUERY: | 3818 | GGAGCATTTCCTGTCCCAGCTGCAACGGCCTTGCTGAAGGCAACAAGCTGCTGGCCCCAG | 3877 |
| | | \| \|\|\|\|\| \|\|\|\|\|\|\|\| \|\|\|\|\| \|\| \|\| \|\|\|\|\| \| \|\|\| \|\| \|\|\|\|\| \|\|\|\|\|\|\| \| | |
| SBJCT: | 3746 | GAAGCATATCCTGTCCTAGCTGCAATGGTCTTGCAGAAGGAAATAAGCTTTTGGCCCCTG | 3805 |
| QUERY: | 3878 | TGGCTCTGGCTGTTGGAATCGATGGGAGCCTCTATGTGGGTGACTTCAATTACATCCGAC | 3937 |
| | | \| \|\| \|\|\|\|\| \|\| \|\|\|\|\| \|\|\|\|\| \|\|\|\|\|\|\| \|\|\| \|\| \|\| \|\| \|\|\|\|\|\|\|\| \|\| \| | |
| SBJCT: | 3806 | TAGCACTGGCAGTGGGAATTGATGGAAGCCTCTTTGTTGGAGATTTTAATTACATTCGGC | 3865 |
| QUERY: | 3938 | GCATCTTTCCCTCTCGAAATGTGACCAGCATCTTGGAGTTACGAAATAAAGAGTTTAAAC | 3997 |
| | | \| \|\|\|\|\| \|\| \|\| \| \|\|\|\|\|\|\|\| \|\|\|\|\| \|\|\|\|\|\| \| \|\|\|\|\|\|\|\|\|\|\|\|\|\|\|\|\|\| | |
| SBJCT: | 3866 | GTATCTTCCCATCCAGGAATGTGACTAGCATATTGGAGCTGAGAAATAAAGAGTTTAAAC | 3925 |
| QUERY: | 3998 | ATAGCAACAACCCAGCACACAAGTACTACTTGGCAGTGGACCCCGTGTCCGGCTCGCTCT | 4057 |
| | | \|\|\|\|\|\|\|\|\|\| \|\| \|\| \|\|\|\|\| \|\|\|\|\| \|\|\|\| \|\|\|\|\|\|\|\|\|\|\| \|\| \|\|\|\|\| \|\| \| | |
| SBJCT: | 3926 | ATAGCAACAATCCTGCTCACAAATACTATCTGGCCGTGGACCCCGTTTCGGGCTCCCTGT | 3985 |
| QUERY: | 4058 | ACGTGTCCGACACCAACAGCAGGAGAATCTACCGCGTCAAGTCTCTGAGTGGAACCAAAG | 4117 |
| | | \|\|\|\| \|\| \|\|\|\|\|\|\|\|\|\|\|\| \| \| \|\| \|\|\| \|\|\|\|\| \|\|\|\|\| \| \|\|\| \|\| \|\|\|\| | |
| SBJCT: | 3986 | ACGTATCAGACACCAACAGCCGACGGATATACAAAGTCAAATCTCTTACTGGCACGAAAG | 4045 |
| QUERY: | 4118 | ACCTGGCTGGGAATTCGGAAGTTGTGGCAGGGACGGGAGAGCAGTGTCTACCCTTTGATG | 4177 |
| | | \|\|\|\|\|\|\|\|\|\| \|\|\|\|\| \|\|\|\|\| \|\| \|\| \|\|\|\|\| \|\|\|\|\|\|\|\| \|\| \|\| \|\|\|\|\|\|\|\|\|\| | |
| SBJCT: | 4046 | ACCTGGCTGGTAATTCTGAAGTGGTAGCGGGGACTGGAGAGCAATGCCTGCCCTTTGATG | 4105 |
| QUERY: | 4178 | AAGCCCGCTGCGGGGATGGAGGGAAGGCCATAGATGCAACCCTGATGAGCCCGAGAGGTA | 4237 |
| | | \|\|\|\|\| \| \|\| \|\| \|\|\|\|\|\|\|\|\|\|\| \|\| \| \|\| \|\|\|\|\|\|\|\| \|\|\|\|\| \|\| \|\|\|\| \| | |
| SBJCT: | 4106 | AAGCCAGATGTGGAGATGGAGGGAAAGCAGTGGACGCAACCCTAATGAGTCCTCGAGGAA | 4165 |
| QUERY: | 4238 | TTGCAGTAGACAAGAATGGGCTCATGTACTTTGTCGATGCCACCATGATCCGGAAGGTTG | 4297 |
| | | \|\|\|\|\|\|\| \|\| \|\|\| \|\|\|\| \|\|\|\|\|\|\|\| \|\|\|\|\| \|\|\|\|\|\|\|\| \|\|\|\|\| \|\| \|\| \|\| \| | |
| SBJCT: | 4166 | TTGCAGTGGATAAGTATGGACTCATGTATTTTGTTGATGCCACTATGATTCGAAAAGTGG | 4225 |
| QUERY: | 4298 | ACCAGAATGGAATCATCTCCACCCTGCTGGGCTCCAATGACCTCACTGCCGTCCGGCCGC | 4357 |
| | | \| \|\|\|\|\|\|\|\|\|\|\| \|\| \|\| \|\| \|\|\|\|\|\|\|\|\|\|\|\|\|\|\|\|\|\|\|\| \|\|\|\|\|\|\|\|\|\|\| \|\| \| | |
| SBJCT: | 4226 | ATCAGAATGGAATTATATCAACTCTGCTGGGCTCCAATGACCTAACTGCCGTCCGACCTC | 4285 |
| QUERY: | 4358 | TGAGCTGTGATTCCAGCATGGATGTAGCCCAGGTTCGTCTGGAGTGGCCAACAGACCTTG | 4417 |
| | | \| \|\|\|\|\|\|\|\|\|\|\|\|\|\|\|\|\|\|\|\|\|\|\| \|\|\|\|\|\| \|\| \|\|\|\|\|\|\|\|\|\|\| \|\| \|\| \|\| \| | |
| SBJCT: | 4286 | TAAGCTGTGATTCCAGCATGGATGTCAGCCAGGTACGGCTGGAGTGGCCTACTGATCTCG | 4345 |
| QUERY: | 4418 | CTGTCAATCCCATGGATAACTCCTTGTATGTTCTAGAGAACAATGTCATCCTTCGAATCA | 4477 |
| | | \|\|\|\|\| \|\|\|\|\|\|\|\|\|\| \|\|\|\|\| \| \|\|\|\|\| \|\|\|\|\|\|\|\|\|\|\|\|\|\| \|\| \| \|\| \|\|\|\| | |
| SBJCT: | 4346 | CTGTCGATCCCATGGACAACTCACTTTATGTCCTAGAGAACAATGTTATTTTACGGATCA | 4405 |
| QUERY: | 4478 | CCGAGAACCACCAAGTCAGCATCATTGCGGGACGCCCCATGCACTGCCAAGTTCCTGGCA | 4537 |
| | | \| \|\| \|\|\|\|\| \|\|\|\|\| \|\|\|\|\| \|\|\|\|\| \|\|\|\|\|\|\|\|\|\|\|\|\|\|\|\|\|\|\|\| \|\|\|\|\|\|\|\| \| | |
| SBJCT: | 4406 | CAGAAAACCATCAAGTTAGCATTATTGCTGGACGCCCCATGCACTGCCAGGTTCCTGGTA | 4465 |
| QUERY: | 4538 | TTGACTACTCACTCAGCAAACTAGCCATTCACTCTGCCCTGGAGTCAGCCAGTGCCATTG | 4597 |
| | | \| \|\|\|\|\|\|\|\| \|\| \|\|\|\|\|\|\|\| \|\| \|\|\|\|\| \|\| \|\| \|\| \|\| \|\|\|\|\|\|\|\|\|\|\|\|\|\|\|\| | |
| SBJCT: | 4466 | TAGACTACTCTCTTAGCAAACTGGCTATTCATTCCGCACTTGAATCAGCCAGTGCCATTG | 4525 |
| QUERY: | 4598 | CCATTTCTCACACTGGGGTCCTCTACATCACTGAGACAGATGAGAAGAAGATTAACCGTC | 4657 |
| | | \|\|\|\| \|\| \|\|\|\|\| \|\| \|\| \|\| \|\|\|\|\|\|\| \|\|\|\|\|\|\|\|\|\|\|\| \|\| \|\| \|\|\|\|\| \|\| \| | |
| SBJCT: | 4526 | CCATCTCACACACAGGAGTTCTTTACATCAGTGAGACAGATGAAAAAAAAATTAATCGGC | 4585 |
| QUERY: | 4658 | TACGCCAGGTAACAACCAACGGGGAGATCTGCCTTTTAGCTGGGGCAGCCTCGGACTGCG | 4717 |
| | | \|\|\|\|\|\|\|\|\|\|\|\|\| \|\|\|\|\| \|\| \|\| \|\| \|\|\|\|\|\| \| \|\| \|\|\|\|\|\|\|\| \|\| \|\|\|\|\| \| | |
| SBJCT: | 4586 | TACGCCAGGTAACTACCAATGGAGAAATATGCCTTCTTGCAGGGGCAGCTTCAGACTGTG | 4645 |
| QUERY: | 4718 | ACTGCAAAAACGATGTCAATTGCAACTGCTATTCAGGAGATGATGCCTACGCGACTGATG | 4777 |
| | | \| \|\|\|\|\|\|\|\| \|\|\|\|\|\|\|\| \|\| \|\| \|\|\|\|\|\|\|\| \|\| \|\|\|\|\|\|\| \|\| \|\| \|\|\|\|\|\|\| | |
| SBJCT: | 4646 | ATTGCAAAAATGATGTCAACTGTAATTGCTATTCTGGGGATGATGGGTATGCCACTGATG | 4705 |
| QUERY: | 4778 | CCATCTTGAATTCCCCATCATCCTTAGCTGTAGCTCCAGATGGTACCATTTACATTGCAG | 4837 |
| | | \|\|\|\|\|\|\| \|\|\|\|\| \|\|\|\|\| \|\|\|\|\|\|\|\|\|\|\| \|\| \|\|\|\|\|\|\|\|\|\|\|\|\|\| \|\|\|\|\| \|\| \| | |
| SBJCT: | 4706 | CCATCTTAAATTCACCATCTTCCTTAGCTGTGGCCCCAGATGGTACCATCTACATAGCTG | 4765 |
| QUERY: | 4838 | ACCTTGGAAATATTCGGATCAGGGCGGTCAGCAAGAACAAGCCTGTTCTTAATGCCTTCA | 4897 |
| | | \| \|\| \|\|\|\|\|\|\|\| \|\| \|\| \|\|\|\|\| \|\|\|\|\| \|\| \|\|\|\| \|\|\| \|\|\|\|\|\|\|\| \| \|\| \| | |
| SBJCT: | 4766 | ATCTCGGAAATATCCGCATTAGGGCTGTCAGTAAAAACAGGCCCATTCTTAATTCTTTTA | 4825 |
| QUERY: | 4898 | ACCAGTATGAGGCTGCATCCCCCGGAGAGCAGGAGTTATATGTTTTCAACGCTGATGGCA | 4957 |
| | | \|\|\|\| \|\|\|\|\| \|\|\|\|\|\|\|\| \|\| \|\|\|\|\| \|\|\|\|\|\| \| \|\|\|\|\| \|\|\|\|\| \|\|\|\|\|\|\|\| \| | |
| SBJCT: | 4826 | ACCAATATGAAGCTGCATCTCCAGGAGAACAGGAGCTGTATGTCTTCAATGCTGATGGGA | 4885 |
| QUERY: | 4958 | TCCACCAATACACTGTGAGCCTGGTGACAGGGGAGTACTTGTACAATTTCACATATAGTA | 5017 |
| | | \| \|\|\|\|\| \|\|\|\|\|\| \| \|\|\|\|\| \|\| \|\| \|\|\|\|\|\|\|\|\|\|\|\|\|\|\|\|\|\|\|\|\|\|\| \|\|\|\|\| \| | |
| SBJCT: | 4886 | TTCACCAGTACACTCTCAGCCTTGTTACCGGGGAGTACTTGTACAATTTCACCTATAGCA | 4945 |
| QUERY: | 5018 | CTGACAATGATGTCACTGAATTGATTGACAATAATGGGAATTCCCTGAAGATCCGTCGGG | 5077 |
| | | \|\|\| \|\| \|\|\|\|\|\|\|\| \|\| \|\|\|\| \|\|\|\| \|\|\|\|\| \|\| \|\|\| \|\|\|\|\| \|\|\|\|\|\|\|\|\| | |
| SBJCT: | 4946 | GTGATAACGATGTCACCGAGGTGATGGACAGCAATGGCAACTCCTTGAAGGTCCGTCGGG | 5005 |
| QUERY: | 5078 | ACAGCAGTGGCATGCCCCGTCACCTGCTCATGCCTGACAACCAGATCATCACCCTCACCG | 5137 |
| | | \| \|\|\| \|\| \|\|\|\|\|\|\|\| \|\| \| \|\| \|\|\|\|\|\|\|\| \|\| \|\|\|\|\| \|\|\|\| \|\| \|\|\| | |
| SBJCT: | 5006 | ATGCCAGCGGAATGCCCCGCCATTTACTGATGCCTGATAATCAGATTGTCACGCTGGCCG | 5065 |
| QUERY: | 5138 | TGGGCACCAATGGAGGCCTCAAAGTCGTGTCCACACAGAACCTGGAGCTTGGTCTCATGA | 5197 |
| | | \| \|\|\|\|\| \|\|\|\|\| \|\| \|\|\|\|\|\| \| \|\| \|\| \|\| \|\|\|\| \|\|\|\|\|\| \|\|\|\|\| \| \|\|\|\| | |
| SBJCT: | 5066 | TTGGCACTAATGGTGGACTCAAACTAGTCTCAACGCAGACCCTGGAACTTGGATTAATGA | 5125 |
| QUERY: | 5198 | CCTATGATGGCAACACTGGGCTCCTGGCCACCAAGAGCGATGAAACAGGATGGACGACTT | 5257 |
| | | \| \|\|\| \| \|\| \|\|\|\| \|\|\| \|\|\| \| \|\| \|\| \|\|\|\|\| \|\|\|\|\|\|\|\|\|\|\|\|\|\|\|\|\| \|\| \| | |
| SBJCT: | 5126 | CTTATAACGGAAACAGTGGTCTCTTAGCAACGAAGAGTGATGAAACAGGATGGACAACAT | 5185 |
| QUERY: | 5258 | TCTATGACTATGACCACGAAGGCCGCCTGACCAACGTGACGCGCCCCACGGGGGTGGTAA | 5317 |
| | | \| \|\|\|\|\|\|\|\|\|\|\| \|\| \|\|\|\|\| \|\|\|\|\|\|\|\|\|\|\| \|\| \|\| \|\| \|\|\|\|\| \|\| \|\|\|\|\|\|\| | |
| SBJCT: | 5186 | TTTATGACTATGATCATGAAGGGCGCCTGACCAATGTAACACGTCCCACTGGAGTGGTAA | 5245 |
| QUERY: | 5318 | CCAGTCTGCACCGGGAAATGGAGAAATCTATTACCATTGACATTGAGAACTCCAACCGTG | 5377 |
| | | \| \|\| \|\| \|\| \|\| \|\|\|\|\|\|\|\| \|\| \|\|\|\|\|\|\|\|\|\|\| \|\|\|\|\|\|\|\|\|\|\| \|\| \|\| \|\| \| | |
| SBJCT: | 5246 | CTAGCCTTCATCGAGAAATGGAAAAGTCTATTACCATCGACATTGAGAATTCTAATCGGG | 5305 |
| QUERY: | 5378 | ATGATGACGTCACTGTCATCACCAACCTCTCTTCAGTAGAGGCCTCCTACACAGTGGTAC | 5437 |
| | | \|\|\|\|\|\|\| \|\|\|\|\| \|\|\|\|\|\|\|\| \|\| \|\|\|\|\| \|\| \|\| \|\|\|\|\| \|\|\|\|\| \|\|\|\|\| \|\| \| | |
| SBJCT: | 5306 | ATGATGATGTCACGGTCATCACAAATCTCTCCTCTGTGGAGGCTTCCTATACAGTTGTTC | 5365 |
| QUERY: | 5438 | AAGATCAAGTTCGGAACAGCTACCAGCTCTGTAATAATGGTACCCTGAGGGTGATGTATG | 5497 |
| | | \|\|\|\|\|\|\|\|\|\| \|\|\|\|\|\|\|\|\|\|\|\|\|\|\|\|\|\|\|\|\|\|\|\|\|\|\|\|\|\|\| \|\|\|\| \|\|\|\|\|\|\|\|\|\| | |
| SBJCT: | 5366 | AAGATCAAGTGAGGAACAGCTACCAGCTCTGTAATAATGGTACTTTGAGAGTGATGTATG | 5425 |
| QUERY: | 5498 | CTAATGGGATGGGTATCAGCTTCCACAGCGAGCCCCATGTCCTAGCGGGCACCATCACCC | 5557 |
| | | \| \|\|\|\|\| \|\|\| \|\|\|\| \|\|\|\|\| \|\|\|\|\|\|\|\| \|\| \|\|\|\|\|\|\|\| \|\| \|\| \|\| \| \|\| \| | |
| SBJCT: | 5426 | CCAATGGCATGAGTATTAGCTTTCACAGCGAACCTCATGTCCTGGCTGGGACAGTAACTC | 5485 |
| QUERY: | 5558 | CCACCATTGGACGCTGCAACATCTCCCTGCCTATGGAGAATGGCTTAAACTCCATTGAGT | 5617 |
| | | \|\|\|\|\|\|\| \|\|\|\|\| \|\| \|\| \|\| \|\| \|\| \|\| \|\|\|\|\|\|\|\|\|\|\| \|\| \|\|\|\|\| \|\|\|\|\| \| | |
| SBJCT: | 5486 | CCACCATAGGACGATGTAATATTTCTCTACCAATGGAGAATGGTTTGAACTCAATTGAAT | 5545 |
| QUERY: | 5618 | GGCGCCTAAGAAAGGAACAGATTAAAGGCAAAGTCACCATCTTTGGCAGGAAGCTCCGGG | 5677 |
| | | \|\|\|\| \|\| \|\| \|\| \|\|\|\|\|\|\|\|\|\|\|\|\|\|\|\|\|\|\|\| \|\| \| \|\|\|\|\| \|\| \|\|\|\|\|\| \|\|\| | |
| SBJCT: | 5546 | GGCGTCTGAGGAAAGAACAGATTAAAGGCAAAGTGACTGTGTTTGGAAGAAAGCTCAGGG | 5605 |
| QUERY: | 5678 | TCCATGGAAGAAATCTCTTGTCCATTGACTATGATCGAAATATTCGGACTGAAAAGATCT | 5737 |
| | | \| \|\|\|\|\|\|\|\| \|\|\| \| \|\|\|\|\|\|\|\|\|\| \|\| \|\| \|\| \|\|\|\|\| \|\| \|\| \|\|\|\|\| \|\|\|\| | |
| SBJCT: | 5606 | TTCATGGAAGGAATTTGCTGTCCATTGATTACGACCGGAATATACGCACAGAAAAAATCT | 5665 |
| QUERY: | 5738 | ATGATGACCACCGGAAGTTCACCCTGAGGATCATTTATGACCAGGTGGGCCGCCCCTTCC | 5797 |
| | | \| \|\|\|\|\| \|\|\|\|\| \|\|\|\|\|\|\|\|\|\|\|\|\|\|\|\|\| \|\|\|\|\| \|\| \|\|\| \|\|\|\| \|\| \|\|\|\|\|\|\| | |
| SBJCT: | 5666 | ACGATGATCACCGCAAGTTCACCCTGAGGATAATTTACGATCAGCTGGGACGGCCCTTCC | 5725 |
| QUERY: | 5798 | TCTGGCTGCCCAGCAGCGGGCTGGCAGCTGTCAACGTGTCATACTTCTTCAATGGGCGCC | 5857 |
| | | \|\|\|\|\|\|\|\|\|\|\|\|\|\|\|\|\|\|\| \|\|\|\|\| \|\| \|\|\|\|\|\|\|\|\|\|\| \|\| \|\|\|\|\|\|\|\| \|\|\|\|\|\|\| | |
| SBJCT: | 5726 | TCTGGCTGCCCAGCAGCGGCCTGGCTGCCGTCAACGTGTCCTATTTCTTCAACGGGCGCC | 5785 |
| QUERY: | 5858 | TGGCTGGGCTTCAGCGTGGGGCCATGAGCGAGAGGACAGACATCGACAAGCAAGGCCGCA | 5917 |
| | | \|\|\|\|\|\|\|\|\|\|\|\|\|\|\|\| \|\| \|\|\|\|\|\|\|\|\|\|\| \|\|\|\|\|\|\|\|\|\|\|\|\|\|\|\|\|\|\|\|\|\|\|\| \| \| | |
| SBJCT: | 5786 | TGGCTGGGCTTCAGCGCGGAGCCATGAGCGAAAGGACAGACATCGACAAGCAAGGCAGGA | 5845 |
| QUERY: | 5918 | TCGTGTCCCGCATGTTCGCTGACGGGAAAGTGTGGAGCTACTCCTACCTTGACAAGTCCA | 5977 |
| | | \|\| \| \|\| \|\|\|\|\|\|\|\| \|\| \|\| \|\|\|\|\| \|\| \|\|\|\|\| \|\|\| \|\|\|\|\|\|\| \|\| \|\| \|\|\|\| | |
| SBJCT: | 5846 | TCATATCGCGCATGTTTGCAGATGGGAAGGTTTGGAGTTACACCTACCTAGAAAAATCCA | 5905 |
| QUERY: | 5978 | TGGTCCTCCTGCTTCAGAGCCAACGTCAGTATATATTTGAGTATGACTCCTCTGACCGCC | 6037 |
| | | \|\|\|\| \|\| \|\|\|\|\|\|\|\|\|\|\|\|\|\| \|\| \|\|\|\|\| \|\| \|\|\|\|\|\|\|\|\|\|\| \|\| \|\| \|\|\|\|\| \| | |
| SBJCT: | 5906 | TGGTACTACTGCTTCAGAGCCAGCGGCAGTACATCTTTGAGTATGATTCTTCAGACCGGC | 5965 |
| QUERY: | 6038 | TCCTTGCCGTCACCATGCCCAGCGTGGCCCGGCACAGCATGTCCACACACACCTCCATCG | 6097 |
| | | \|\|\| \|\|\| \|\| \|\| \|\|\|\|\| \|\| \|\| \|\| \|\|\|\|\| \|\|\|\|\|\|\|\| \|\| \|\|\|\|\| \|\| \| \| | |
| SBJCT: | 5966 | TCCATGCTGTTACTATGCCTAGTGTTGCTCGGCATAGCATGTCAACTCACACGTCTGTTG | 6025 |
| QUERY: | 6098 | GCTACATCCGTAATATTTACAACCCGCCTGAAAGCAATGCTTCGGTCATCTTTGACTACA | 6157 |
| | | \|\|\|\|\|\|\| \| \|\|\|\|\|\|\|\| \|\| \|\| \|\|\|\|\|\|\|\|\|\|\| \|\| \|\| \|\| \|\| \|\|\|\|\| \|\|\|\| | |
| SRJCT: | 6026 | GCTACATTAGGAATATTTATAATCCTCCTGAAAGCAACGCATCAGTGATTTTTGATTACA | 6085 |
| QUERY: | 6158 | GTGATGACGGCCGCATCCTGAAGACCTCCTTTTTGGGCACCGGACGCCAGGTGTTCTACA | 6217 |
| | | \|\|\|\|\|\|\| \|\| \| \|\| \|\|\|\| \|\| \|\| \|\|\|\|\| \|\| \|\| \|\| \|\| \|\| \|\| \|\| \|\|\|\| | |
| SBJCT: | 6086 | GTGATGATGGGAGGATTTTGAAAACATCATTTTTAGGTACTGGTCGACAAGTCTTTTACA | 6145 |
| QUERY: | 6218 | AGTATGGGAAACTCTCCAAGTTATCAGAGATTGTCTACGACAGTACCGCCGTCACCTTCG | 6277 |
| | | \|\|\|\|\|\|\| \|\| \|\| \|\|\|\|\| \|\|\|\|\| \|\| \|\|\|\|\| \|\| \|\|\|\|\|\|\|\| \|\| \|\| \|\| \|\| \| | |
| SBJCT: | 6146 | AGTATGGAAAGCTATCCAAATTATCTGAAATTGTTTATGACAGTACTGCGGTTACTTTTG | 6205 |
| QUERY: | 6278 | GGTATGACGAGACCACTGGTGTCTTGAAGATGGTCAACCTCCAAAGTGGGGGCTTCTCCT | 6337 |
| | | \| \|\|\|\|\| \|\| \|\| \|\| \|\|\|\|\|\| \| \|\| \|\|\|\|\| \|\| \| \|\|\|\|\|\|\|\| \|\| \|\| \|\| \| | |
| SBJCT: | 6206 | GATATGATGAAACTACAGGTGTCCTAAAAATGGTGAATTTGCAAAGTGGAGGATTTTCTT | 6265 |
| QUERY: | 6338 | GCACCATCAGGTACCGGAAGATTGGCCCCCTGGTGGACAAGCAGATCTACAGGTTCTCCG | 6397 |
| | | \| \|\| \|\|\| \| \|\| \|\| \|\| \|\|\|\|\|\|\|\| \|\| \|\| \|\|\|\|\| \|\| \|\|\|\|\|\|\|\| \|\|\|\|\| \| | |
| SBJCT: | 6266 | GTACAATCCGCTATCGTAAAATTGGCCCTCTTGTTGACAAACAAATCTACAGATTCTCTG | 6325 |
| QUERY: | 6398 | AGGAAGGCATGGTCAATGCCAGGTTTGACTACACCTATCATGACAACAGCTTCCGCATCG | 6457 |
| | | \| \|\|\|\|\| \|\|\|\|\|\|\|\|\|\|\| \|\|\|\|\|\|\|\| \|\| \|\| \|\|\|\|\| \|\|\|\|\| \|\| \|\| \|\|\|\|\| \| | |
| SBJCT: | 6326 | AAGAAGGTATGGTCAATGCAAGGTTTGATTATACATATCACGACAATAGTTTTCGCATTG | 6385 |
| QUERY: | 6458 | CAAGCATCAAGCCCGTCATAAGTGAGACTCCCCTCCCCGTTGACCTCTACCGCTATGATG | 6517 |
| | | \|\|\|\|\|\|\|\|\|\| \|\|\| \|\|\|\|\|\|\|\|\|\|\|\|\|\|\|\| \|\| \|\| \|\|\|\|\| \|\| \|\|\|\|\| \|\|\|\|\|\|\| | |
| SBJCT: | 6386 | CAAGCATCAAACCCATCATAAGTGAGACTCCTCTTCCAGTTGATCTTTACCGTTATGATG | 6445 |
| QUERY: | 6518 | AGATTTCTGGCAAGGTGGAACACTTTGGTAAGTTTGGAGTCATCTATTATGACATCAACC | 6577 |
| | | \|\|\|\|\|\|\|\|\|\|\|\|\| \|\| \|\| \|\| \|\|\|\|\| \|\| \|\|\|\|\|\|\|\| \|\| \|\|\|\|\|\|\|\| \|\| \|\| \| | |
| SBJCT: | 6446 | AGATTTCTGGCAAAGTTGAGCATTTTGGCAAATTTGGAGTTATTTATTATGATATAAATC | 6505 |
| QUERY: | 6578 | AGATCATCACCACTGCCGTGATGACCCTCAGCAAACACTTCGACACCCATGGGCGGATCA | 6637 |
| | | \| \|\| \|\| \|\| \|\| \|\| \|\| \|\|\|\|\| \|\| \|\| \|\| \|\|\|\|\| \|\| \|\|\|\|\| \|\| \|\| \|\| \| | |
| SBJCT: | 6506 | AAATTATTACTACAGCAGTTATGACACTGAGTAAGCACTTTGATACCCACGGACGCATTA | 6565 |
| QUERY: | 6638 | AGGAGGTCCAGTATGAGATGTTCCGGTCCCTCATGTACTGGATGACGGTGCAATATGACA | 6697 |
| | | \| \|\| \|\| \|\| \|\|\|\|\|\|\|\|\|\|\|\|\|\| \|\|\|\|\| \|\|\|\|\|\|\|\|\|\|\|\|\|\| \|\|\|\|\|\|\|\|\|\|\|\|\| | |
| SBJCT: | 6566 | AAGAAGTTCAATATGAGATGTTCCGATCCCTGATGTACTGGATGACTGTGCAATATGACA | 6625 |
| QUERY: | 6698 | GCATGGGCAGGGTGATCAAGAGGGAGCTAAAACTGGGGCCCTATGCCAATACCACGAAGT | 6757 |
| | | \|\|\|\|\|\|\| \|\| \|\| \| \|\| \|\| \|\| \|\| \|\|\|\|\| \|\|\|\|\| \|\|\|\|\|\|\|\| \|\| \|\| \|\|\|\| | |
| SBJCT: | 6626 | GCATGGGAAGAGTAACTAAAAGAGAACTGAAACTTGGGCCGTATGCCAACACAACCAAGT | 6685 |
| QUERY: | 6758 | ACACCTATGACTACGATGGGGACGGGCAGCTCCAGAGCGTGGCCGTCAATGACCGCCCGA | 6817 |
| | | \| \|\|\|\|\|\|\|\| \|\| \|\|\|\|\| \|\| \|\|\|\|\| \| \|\| \|\|\|\|\| \|\| \|\| \|\|\|\|\| \| \|\| \| | |
| SBJCT: | 6686 | ATACCTATGATTATGATGGAGATGGGCAATTGCAAAGCGTAGCAGTAAATGATAGGCCTA | 6745 |
| QUERY: | 6818 | CCTGGCGCTACAGCTATGACCTTAATGGGAATCTCCACTTACTGAACCCAGGCAACAGTG | 6877 |
| | | \|\|\|\|\|\|\| \|\|\|\|\| \|\|\|\|\|\|\|\| \|\|\|\|\| \|\|\|\|\| \|\|\| \| \|\|\|\|\| \|\| \|\| \|\|\|\|\|\|\| | |
| SBJCT: | 6746 | CCTGGCGTTACAGTTATGACCTGAATGGAAATCTTCACCTCCTGAATCCTGGAAACAGTG | 6805 |
| QUERY: | 6878 | TGCGCCTCATGCCCTTGCGCTATGACCTC 6906 | |
| | | \| \|\| \| \|\|\|\|\|\| \|\|\|\|\|\|\| \|\|\|\|\|\| | |
| SBJCT: | 6806 | TTCGATTGATGCCCCTGCGCTACGACCTC 6834 | |

SCORE = 1241 BITS (626), EXPECT = 0.0
IDENTITIES = 1553/1862 (83%)
STRAND = PLUS/PLUS

| | | | |
|---|---|---|---|
| QUERY: | 1486 | AGCAGCATAGACAGTGGTGAAGCAGAAGTTGGTCGGCGGGTAACACAAGAAGTCCCACCA | 1545 |
| | | \|\|\|\|\|\|\|\|\|\|\| \|\|\|\|\| \|\|\| \|\|\|\|\|\|\|\|\|\| \|\| \|\|\| \|\| \|\|\|\|\| \|\| \|\| \|\| | |
| SBJCT: | 1414 | AGCAGCATAGATAGTGGAGAAACAGAAGTTGGCCGCAAGGTCACCCAAGAGGTGCCCCCT | 1473 |
| QUERY: | 1546 | GGGGTGTTTTGGAGGTCACAAATTCACATCAGTCAGCCCCAGTTCTTAAAGTTCAACATC | 1605 |
| | | \|\| \|\|\|\|\| \|\|\| \|\|\|\| \|\| \|\| \|\| \|\|\|\|\| \|\|\|\|\| \|\|\|\|\|\| \| \|\|\|\|\|\|\|\|\|\|\| | |
| SBJCT: | 1474 | GGAGTGTTCTGGCGGTCTCAGATCCATATCAGCCAGCCACAGTTCCTGAAGTTCAACATA | 1533 |
| QUERY: | 1606 | TCCCTCGGGAAGGACGCTCTCTTTGGTGTTTACATAAGAAGAGGACTTCCACCATCTCAT | 1665 |
| | | \|\|\|\|\| \|\|\|\|\|\|\|\| \|\|\|\|\| \|\| \|\|\|\|\|\|\|\| \|\|\|\|\|\|\|\|\|\|\|\|\|\| \|\|\|\|\|\|\|\| \|\|\| | |
| SBJCT: | 1534 | TCCCTAGGGAAGGATGCTCTTTTCGGTGTTTATATAAGAAGAGGACTCCCACCATCACAT | 1593 |
| QUERY: | 1666 | GCCCAGTATGACTTCATGGAACGTCTGGACGGGAAGGAGAAGTGGAGTGTGGTTGAGTCT | 1725 |
| | | \|\| \|\|\|\|\|\|\|\| \|\|\|\|\|\|\|\|\|\|\| \|\|\|\| \|\|\|\|\| \|\|\|\|\| \|\|\|\|\|\|\|\|\|\|\| \|\| \|\| | |
| SBJCT: | 1594 | GCACAGTATQATTTCATGGAACGCTTGGATGGGAAAGAGAAATGGAGTGTGGTGGAATCC | 1653 |
| QUERY: | 1726 | CCCAGGGAACGCCGGAGCATACAGACCTTGGTTCAGAATGAAGCCGTGTTTGTGCAGTAC | 1785 |
| | | \|\| \|\|\|\|\|\|\| \|\| \|\| \|\| \|\|\|\|\| \| \|\|\|\|\|\|\|\|\|\|\| \|\| \|\|\|\|\|\|\|\| \|\|\|\|\|\| | |
| SBJCT: | 1654 | CCACGGGAACGGCGAAGTATTCAGACTCTTGTTCAGAATGAGGCTGTGTTTGTTCAGTAC | 1713 |
| QUERY: | 1786 | CTGGATGTGGGCCTGTGGCATCTGGCCTTCTACAATGATGGAAAAGACAAAGAGATGGTT | 1845 |
| | | \|\|\|\|\|\|\|\|\|\| \|\|\|\|\|\|\| \|\|\|\|\| \|\| \|\|\|\|\|\|\|\|\|\|\| \|\| \|\|\|\|\|\|\|\| \|\|\|\| | |
| SBJCT: | 1714 | TTGGATGTGGGTTTGTGGCACCTGGCGTTTTACAATGATGGCAAGGACAAAGAAGTGGTC | 1773 |
| QUERY: | 1846 | TCCTTCAATACTGTTGTCCTAGATTCAGTGCAGGACTGTCCACGTAACTGCCATGGGAAT | 1905 |
| | | \|\|\|\|\|\|\| \|\|\| \|\|\| \| \| \|\|\|\|\|\|\|\|\|\|\| \|\|\|\|\|\|\|\|\|\|\|\|\|\| \|\| \|\|\|\|\| \|\|\| | |
| SBJCT: | 1774 | TCCTTCAGTACAGTTATTTTGGATTCAGTGCAAGACTGTCCACGTAATTGTCATGGCAAT | 1833 |
| QUERY: | 1906 | GGTGAATGTGTGTCCGGGGTGTGTCACTGTTTCCCAGGATTTCTAGGAGCAGACTGTGCT | 1965 |
| | | \|\| \|\| \|\|\|\|\| \|\| \|\| \|\| \|\| \|\|\|\|\|\|\|\| \|\| \|\|\|\|\|\|\| \|\|\|\|\|\|\|\| \|\|\|\|\|\| | |
| SBJCT: | 1834 | GGCGAGTGTGTTTCTGGTGTCTGCCACTGTTTTCCCGGATTTCATGGAGCAGATTGTGCT | 1893 |
| QUERY: | 1966 | AAAGCTGCCTGCCCTGTCCTGTGCAGTGGGAATGGACAATATTCTAAAGGGACGTGCCAG | 2025 |
| | | \|\|\|\|\|\|\|\|\|\|\|\|\|\| \|\| \|\|\|\|\|\|\|\|\|\|\| \|\|\|\|\| \|\| \|\| \|\| \|\|\|\|\| \|\| \|\|\| \| | |
| SBJCT: | 1894 | AAAGCTGCCTGCCCGGTGCTGTGCAGTGGCAATGGTCAGTACTCCAAAGGAACCTGCTTG | 1953 |
| QUERY: | 2026 | TGCTACAGCGGCTGGAAAGGTGCAGAGTGCGACGTGCCCATGAATCAGTGCATCGATCCT | 2085 |
| | | \|\|\|\|\|\|\|\| \|\|\|\|\|\|\|\|\|\|\|\| \| \|\| \|\| \|\| \|\| \|\|\|\|\| \| \|\|\|\|\| \|\| \|\|\|\|\| | |
| SBJCT: | 1954 | TGCTACAGTGGCTGGAAAGGTCCGGAATGTGATGTACCCATCAGCCAGTGTATTGATCCC | 2013 |
| QUERY: | 2086 | TCCTGCGGGGGCCACGGCTCCTGCATTGATGGGAACTGTGTCTGCTCTGCTGGCTACAAA | 2145 |
| | | \|\| \|\| \|\| \|\| \|\| \|\| \|\|\|\|\|\|\|\| \|\| \|\|\|\|\|\|\|\|\|\|\|\|\|\| \|\| \|\|\|\|\|\| \|\|\| | |
| SBJCT: | 2014 | TCGTGTGGAGGTCATGGTTCCTGCATCGAAGGGAACTGTGTCTGTTCCATTGGCTATAAA | 2073 |
| QUERY: | 2146 | GGCGAGCACTGTGAGGAAGTTGATTGCTTGGATCCCACCTGCTCCAGCCACGGAGTCTGT | 2205 |
| | | \|\| \|\| \|\|\|\|\|\|\|\|\|\|\|\|\|\|\|\|\|\|\|\|\|\| \|\|\|\|\| \|\| \|\|\|\|\|\|\| \|\|\|\|\| \|\|\|\|\|\| | |
| SBJCT: | 2074 | GGAGAAAACTGTGAGGAAGTTGATTGCTTAGATCCAACATGCTCCAATCACGGGGTCTGT | 2133 |
| QUERY: | 2206 | GTGAATGGAGAATGCCTGTGCAGCCCTGGCTGGGGTGGTCTGAACTGTGAGCTGGCGAGG | 2265 |
| | | \|\|\|\|\| \|\|\|\|\|\|\|\| \|\| \|\|\|\|\|\|\|\| \|\|\|\|\|\|\|\|\|\|\| \| \|\|\|\|\|\|\|\|\|\|\| \| \|\| | |
| SBJCT: | 2134 | GTGAACGGAGAATGTCTCTGCAGCCCAGGCTGGGGTGGAATAAACTGTGAGCTTCCCAGA | 2193 |
| QUERY: | 2266 | GTCCAGTGCCCAGACCAGTGCAGTGGGCATGGCACGTACCTGCCTGACACGGGCCTCTGC | 2325 |
| | | \| \|\|\|\|\|\|\|\|\|\|\|\|\|\|\|\|\|\|\|\|\|\|\|\|\|\|\|\|\|\|\|\|\| \|\|\|\|\|\| \|\|\|\|\|\|\| \|\| \|\|\|\|\| | |
| SBJCT: | 2194 | GCCCAGTGCCCAGACCAGTGCAGTGGGCATGGCACATACCTGTCTGACACCGGTCTCTGT | 2253 |
| QUERY: | 2326 | AGCTGCGATCCCAACTGGATGGGTCCCGACTGCTCTGTTGAAGTGTGCTCAGTAGACTGT | 2385 |
| | | \|\|\|\|\|\|\|\|\|\|\|\|\|\|\|\|\|\|\|\|\|\|\|\|\|\|\|\|\|\|\|\|\|\|\| \|\|\|\|\|\|\|\|\|\|\|\|\|\| \|\|\|\|\|\|\|\|\| | |
| SBJCT: | 2254 | AGCTGCGATCCCAACTGGATGGGTCCCGACTGCTCCGTTGAAGTGTGCTCTGTAGACTGT | 2313 |
| QUERY: | 2386 | GGCACTCACGGCGTCTGCATCGGGGGAGCCTGCCGCTGTGAAGAGGGCTGGACAGGCGCA | 2445 |
| | | \|\|\|\|\| \|\| \|\| \|\| \|\|\|\|\| \|\| \|\|\|\|\| \|\| \|\|\|\|\|\|\|\|\|\|\| \|\| \|\|\|\|\|\|\|\| \| | |
| SBJCT: | 2314 | GGCACCCATGGGGTGTGCATTGGCGGAGCGTGTCGCTGTGAAGAAGGGTGGACAGGAGTG | 2373 |
| QUERY: | 2446 | GCGTGTGACCAGCGCGTGTGCCACCCCCGCTGCATTGAGCACGGGACCTGTAAAGATGGC | 2505 |
| | | \|\|\|\|\|\|\|\|\|\|\|\|\|\| \|\|\|\|\| \|\| \|\|\|\|\| \|\| \| \|\|\|\|\|\|\|\| \|\| \|\|\|\|\|\|\|\|\|\|\| | |
| SBJCT: | 2374 | GCGTGTGACCAGCGTGTGTGTCATCCCCGGTGTACAGAGCACGGAACTTGTAAAGATGGG | 2433 |
| QUERY: | 2506 | AAATGTGAATGCCGAGAGGGCTGGAATGGTGAACACTGCACCATTGGTAGGCAAACGGCA | 2565 |
| | | \|\|\|\|\|\|\|\|\|\|\|\| \|\|\|\|\|\|\|\|\|\|\|\|\|\|\|\| \|\| \|\|\|\|\|\|\|\|\|\|\|\|\|\|\|\|\|\|\|\|\|\|\|\| \|\| | |
| SBJCT: | 2434 | AAATGTGAATGCAGAGAGGGCTGGAATGGGGAGCACTGCACCATTGGTAGGCAAACGACA | 2493 |
| QUERY: | 2566 | GGCACCGAAACAGATGGCTGCCCTGACTTGTGCAACGGTAACGGGAGATGCACACTGGGT | 2625 |
| | | \|\|\|\|\|\|\|\|\|\|\|\|\|\|\|\|\|\|\|\|\|\|\|\|\|\|\|\|\|\|\|\|\|\|\| \|\| \|\|\|\|\|\|\|\| \|\|\|\|\| \|\|\|\|\| | |
| SBJCT: | 2494 | GGCACCGAAACAGATGGCTGCCCTGACTTGTGCAATGGCAACGGGAGGTGCACGCTGGGC | 2553 |
| QUERY: | 2626 | CAGAACAGCTGGCAGTGTGTCTGCCAGACCGGCTGGAGAGGGCCCGGATGCAACGTTGCC | 2685 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\| \|\|\|\|\|\|\|\|\|\|\|\|\|\|\| | |
| SBJCT: | 2554 | CAGAACAGCTGGCAGTGTGTCTGCCAGACCGGCTGGAGAGGGCCTGGATGCAACGTTGCC | 2613 |
| QUERY: | 2686 | ATGGAAACTTCCTGTGCTGATAACAAGGATAATGAGGGAGATGGCCTGGTGGATTGTTTG | 2745 |
| | | \|\|\|\|\|\|\|\| \|\|\|\|\|\|\|\| \|\|\|\|\|\|\|\|\|\|\|\|\|\| \|\|\|\|\|\|\|\|\|\|\|\| \|\|\|\| \|\| \|\| \| | |
| SBJCT: | 2614 | ATGGAAACCTCCTGTGCCGATAACAAGGATAACGAGGGAGATGGCTTGGTTGACTGCCTA | 2673 |
| QUERY: | 2746 | GACCCTGACTGCTGCCTGCAGTCAGCCTGTCAGAACAGCCTGCTCTGCCGGGGGTCCCGG | 2805 |
| | | \| \|\|\| \|\| \|\|\|\|\|\|\|\| \|\|\|\|\| \| \|\|\|\|\| \|\|\|\|\|\|\|\|\|\|\| \|\|\|\|\|\|\|\| \|\|\|\|\| | |
| SBJCT: | 2674 | GTCCCAGATTGCTGCCTCCAGTCCACTTGTCAAAACAGCCTGCTGTGCCGGGGTTCCCGC | 2733 |
| QUERY: | 2806 | GACCCACTGGACATCATTCAGCAGGGCCAGACGGATTGGCCCGCAGTGAAGTCCTTCTAT | 2865 |
| | | \|\| \|\| \|\| \|\|\|\|\|\|\|\| \|\| \|\|\| \|\|\|\| \| \| \|\| \|\| \|\| \|\|\|\|\|\|\|\| \|\|\|\|\|\| | |
| SBJCT: | 2734 | GATCCTCTTGACATCATACAACAGAGCCATTCTGGTTCACCAGCTGTGAAGTCATTCTAT | 2793 |
| QUERY: | 2866 | GACCGTATCAAGCTCTTGGCAGGCAAGGATAGCACCCACATCATTCCTGGAGAGAACCCT | 2925 |
| | | \|\| \|\| \|\|\|\|\|\|\|\|\|\|\| \| \|\| \|\|\|\|\| \|\|\|\|\| \|\| \|\|\|\|\|\|\|\| \|\|\|\|\| \|\| \|\| | |
| SBJCT: | 2794 | GATCGAATCAAGCTCTTAGTGGGGAAGGACAGCACTCATATCATTCCAGGAGAAAATCCC | 2853 |
| QUERY: | 2926 | TTCAACAGCAGCTTGGTTTCTCTCATCCGAGGCCAAGTAGTAACTACAGATGGAACTCCC | 2985 |
| | | \|\|\|\|\|\|\|\|\|\|\|\| \| \|\| \|\|\|\|\| \|\| \|\|\|\|\|\|\|\|\|\| \|\| \|\|\|\|\|\|\|\|\|\|\|\|\|\| \|\| | |
| SBJCT: | 2854 | TTCAACAGCAGCCTTGTGTCTCTTATAAGAGGCCAAGTGGTGACTACAGATGGAACGCCT | 2913 |
| QUERY: | 2986 | CTGGTCGGTGTGAACGTGTCTTTTGTCAAGTACCCAAAATACGGCTACACCATCACCCGC | 3045 |
| | | \|\| \|\| \|\| \|\| \|\|\|\|\|\|\|\| \|\|\|\|\|\|\|\|\|\|\| \|\|\|\|\| \|\| \|\|\|\|\| \|\|\|\|\|\|\|\| \|\| | |
| SBJCT: | 2914 | CTAGTTGGGGTCAACGTGTCATTTGTCAAGTATCCAAAGTATGGCTATACCATCACTCGT | 2973 |
| QUERY: | 3046 | CAGGATGGCACGTTCGACCTGATCGCAAATGGAGGTGCTTCCTTGACTCTACACTTTGAG | 3105 |
| | | \|\|\|\|\|\|\|\|\|\| \|\|\| \|\|\| \|\| \| \|\| \|\| \|\| \|\| \| \|\|\| \| \|\|\| \| \|\|\|\|\|\|\|\| | |
| SBJCT: | 2974 | CAGGATGGCATGTTTGACTTGGTTGCTAACGGTGGATCATCCCTAACTTTGCACTTTGAA | 3033 |
| QUERY: | 3106 | CGAGCCCCGTTCATGAGCCAGGAGCGCACTGTGTGGCTGCCGTGGAACAGCTTTTACGCC | 3165 |
| | | \|\| \|\|\|\|\| \|\| \|\|\|\|\| \|\|\|\|\| \| \|\| \|\| \|\|\|\|\|\|\|\|\|\|\|\|\|\|\|\|\|\|\|\| \|\| \|\|\| | |
| SBJCT: | 3034 | CGGGCCCCATTTATGAGTCAGGAAAGGACAGTATGGCTGCCGTGGAACAGCTTCTATGCC | 3093 |
| QUERY: | 3166 | ATGGACACCCTGGTGATGAAGACCGAGGAGAACTCCATCCCCAGCTGTGACCTCAGTGGC | 3225 |
| | | \|\|\|\|\|\|\|\| \|\| \|\| \|\|\|\|\| \|\| \|\|\|\|\|\|\|\|\|\|\|\|\|\| \|\|\|\|\|\|\|\|\|\|\| \|\|\|\|\|\|\|\|\| | |
| SBJCT: | 3094 | ATGGACACGCTTGTAATGAAAACAGAGGAGAACTCCATTCCCAGCTGTGATCTCAGTGGC | 3153 |
| QUERY: | 3226 | TTTGTCCGGCCTGATCCAATCATCATCTCCTCCCCACTGTCCACCTTCTTTAGTGCTGCC | 3285 |
| | | \|\|\|\|\|\| \| \|\|\|\|\|\|\|\|\| \|\|\|\|\|\|\| \|\| \|\| \|\|\|\|\|\|\|\| \|\| \|\|\|\|\| \|\|\|\| \|\|\| | |
| SBJCT: | 3154 | TTTGTCAGACCTGATCCAGTCATCATTTCATCACCACTGTCAACTTTCTTCAGTGATGCT | 3213 |
| QUERY: | 3286 | CCTGGGCAGAATCCCATCGTGCCTGAGACCCAGGTTCTTCATGAAGAAATCGAGCTCCCT | 3345 |
| | | \|\|\|\|\| \| \|\|\|\|\| \|\| \|\| \|\| \|\| \|\|\|\|\|\|\|\|\|\|\|\|\|\|\|\|\|\|\|\|\|\|\| \|\|\| \|\|\|\|\| | |
| SBJCT: | 3214 | CCTGGCCGAAATCCTATTGTACCAGAAACCCAGGTTCTTCATGAAGAAATTGAGGTCCCT | 3273 |
| QUERY: | 3346 | GG 3347 | |
| | | \|\| | |
| SBJCT: | 3274 | GG 3275 | |

SCORE = 547 BITS (276), EXPECT = E − 152
IDENTITIES = 540/628 (85%)
STRAND = PLUS/PLUS

| | | | |
|---|---|---|---|
| QUERY: | 782 | GTCGTCCCATTCCACCTACATCCTCGCCTAGTCTCCTCCCATCTGCTCAGCTGCCTAGCT | 841 |
| | | \|\|\|\|\|\|\|\|\|\|\|\|\|\|\|\|\|\|\|\|\|\|\|\|\|\| \|\|\|\| \|\| \|\|\|\|\|\|\|\|\|\|\|\|\|\|\|\|\|\|\|\| \|\| \| | |
| SBJCT: | 587 | GTCGTCCCATTCCACCTACATCCTCGTCTAGCCTTCTCCCATCTGCTCAGCTGCCCAGTT | 646 |
| QUERY: | 842 | CCCATAATCCTCCACCAGTTAGCTGCCAGATGCCATTGCTAGACAGCAACACCTCCCATC | 901 |
| | | \| \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| \|\| \|\|\|\|\|\|\| | |
| SBJCT: | 647 | CTCATAATCCTCCACCAGTTAGCTGCCAGATGCCATTGCTAGACAGCAATACGTCCCATC | 706 |
| QUERY: | 902 | AAATCATGGACACCAACCCTGATGAGGAATTCTCCCCCAATTCATACCTGCTCAGAGCAT | 961 |
| | | \|\|\|\|\|\|\|\|\|\|\|\|\|\|\|\| \|\|\|\|\| \|\|\|\|\| \|\|\|\|\| \|\| \|\|\|\|\|\|\|\|\|\|\| \|\| \|\|\|\|\|\|\| | |
| SBJCT: | 707 | AAATCATGGACACCAATCCTGACGAGGAGTTCTCTCCTAATTCATACCTACTAAGAGCAT | 766 |
| QUERY: | 962 | GCTCAGGGCCCCAGCAAGCCTCCAGCAGTGGCCCTCCGAACCACCACAGCCAGTCGACTC | 1021 |
| | | \| \|\|\|\|\|\|\|\| \|\|\|\|\| \|\| \|\|\|\|\|\|\|\|\|\|\|\|\|\|\| \| \|\|\|\|\| \|\|\|\|\|\|\|\|\|\|\| \|\| \| | |
| SBJCT: | 767 | GTTCAGGGCCACAGCAGGCATCCAGCAGTGGCCCTTCAACCATCACAGCCAGTCAACGC | 826 |
| QUERY: | 1022 | TGAGGCCCCCTCTCCCACCCCCTCACAACCACACGCTGTCCCATCACCACTCGTCCGCCA | 1081 |
| | | \|\|\|\|\|\|\| \|\|\|\|\|\|\|\| \|\| \|\|\|\|\|\|\|\|\|\|\|\| \|\|\|\|\|\|\|\|\|\|\|\|\| \|\|\|\|\|\|\|\| \|\|\|\| | |
| SBJCT: | 827 | TGAGGCCACCTCTCCCCCCTCCTCACAACCACTCGCTGTCCCATCATCACTCGTCTGCCA | 886 |
| QUERY: | 1082 | ACTCCCTCAACAGGAACTCACTGACCAATCGGCGGAGTCAGATCCACGCCCCGGCCCCAG | 1141 |
| | | \|\|\|\|\|\|\|\|\|\|\|\|\|\|\|\|\|\|\| \|\| \|\|\|\|\| \|\| \|\| \| \|\|\|\|\|\|\|\|\|\|\| \|\| \|\| \|\| \| | |
| SBJCT: | 887 | ACTCCCTCAACAGGAACTCGCTCACCAACCGCCGCAACCAGATCCACGCGCCTGCTCCCG | 946 |
| QUERY: | 1142 | CGCCCAATGACCTGGCCACCACACCAGAGTCCGTTCAGCTTCAGGACAGCTGGGTGCTAA | 1201 |
| | | \| \|\|\|\|\|\|\|\|\|\|\|\|\|\| \|\|\|\|\| \|\| \|\|\|\|\| \|\| \|\|\|\|\| \|\|\|\|\|\|\|\|\|\|\|\|\|\|\|\|\| \| | |
| SBJCT: | 947 | CTCCCAATGACCTGGCGACCACGCCTGAGTCTGTGCAGCTGCAGGACAGCTGGGTGCTCA | 1006 |
| QUERY: | 1202 | ACAGCAACGTGCCACTGGAGACCCGGCACTTCCTCTTCAAGACCTCCTCGGGGAGCACAC | 1261 |
| | | \|\|\|\|\|\|\|\|\|\|\|\|\| \|\|\|\|\|\|\|\|\| \|\|\|\| \|\|\| \| \|\| \|\|\|\|\| \|\| \|\| \|\| \| \|\| \| | |
| SBJCT: | 1007 | ACAGCAACGTGCCGCTGGAGACCAGGCATTTCTTGTTTAAGACATCTTCTGGAACGACTC | 1066 |
| QUERY: | 1262 | CCTTGTTCAGCAGCTCTTCCCCGGGATACCCTTTGACCTCAGGAACGGTTTACACGCCCC | 1321 |
| | | \| \|\|\|\|\|\|\| \|\|\|\|\|\|\|\|\|\|\| \|\| \|\|\|\|\| \|\|\|\|\|\|\|\|\|\|\|\|\| \|\|\|\|\| \|\| \|\| \| | |
| SBJCT: | 1067 | CGCTGTTCAGTAGCTCTTCCCCTGGCTACCCACTGACCTCAGGAACAGTTTATACTCCAC | 1126 |
| QUERY: | 1322 | CGCCCCGCCTGCTGCCCAGGAATACTTTCTCCAGGAAGGCTTTCAAGCTGAAGAAGCCCT | 1381 |
| | | \| \|\|\| \| \|\|\| \| \|\| \|\| \|\|\|\|\| \|\| \|\|\|\|\|\|\|\| \|\| \|\|\|\|\|\|\|\|\|\|\| \|\|\|\|\|\|\| | |
| SBJCT: | 1127 | CTCCCAGGCTGTTACCTAGAAATACATTTTCCAGGAATGCATTCAAGCTGAAAAGCCCT | 1186 |
| QUERY: | 1382 | CCAAATACTGCAGCTGGAAATGTGCTGC | 1409 |
| | | \|\|\|\| \|\| \|\| \|\|\|\|\|\|\|\|\|\|\|\|\|\|\|\|\| | |
| SBJCT: | 1187 | CCAAGTATTGTAGCTGGAAATGTGCTGC | 1214 |

SCORE = 391 BITS (197), EXPECT = E − 105
IDENTITIES = 593/725 (81%)
STRAND = PLUS/PLUS

| | | | |
|---|---|---|---|
| QUERY: | 7156 | CATGTCTACAATCACTCCAACTCGGAGATTACCTCACTGTACTACGACCTCCAGGGCCAC | 7215 |
| | | \|\|\|\|\|\|\|\|\|\|\|\|\|\| \|\|\|\|\| \|\| \|\| \|\|\|\|\|\|\|\| \|\|\|\|\| \|\| \|\| \|\| \|\| \|\|\|\|\|\| | |
| SBJCT: | 7084 | CATGTCTACAATCATTCCAATTCAGAAATTACCTCTCTGTATTATGATCTGCAAGGCCAC | 7143 |
| QUERY: | 7216 | CTCTTTGCCATGGAGAGCAGCAGTGGGGAGGAGTACTATGTTGCCTCTGATAACACAGGG | 7275 |
| | | \|\|\|\|\|\|\|\| \|\|\|\|\|\|\|\| \|\|\|\|\|\|\|\|\|\|\| \|\| \|\| \|\|\|\|\| \|\|\|\|\| \|\|\|\|\|\|\|\| \|\| | |
| SBJCT: | 7144 | CTCTTTGCAATGGAGAGTAGCAGTGGGGAAGAATATTATGTCGCCTCCGATAACACGGGC | 7203 |
| QUERY: | 7276 | ACTCCTCTGGCTGTGTTCAGCATCAACGGCCTCATGATCAAACAGCTGCAGTACACGGCC | 7335 |
| | | \|\|\|\|\| \|\| \|\| \|\| \|\|\|\|\|\|\|\|\|\|\| \|\|\|\|\|\|\|\|\|\|\|\|\|\|\|\|\|\|\|\| \|\|\|\|\|\|\|\| \|\| | |
| SBJCT: | 7204 | ACTCCGCTAGCCGTATTCAGCATCAATGGCCTCATGATCAAACAGCTTCAGTACACTGCA | 7263 |
| QUERY: | 7336 | TATGGGGAGATTTATTATGACTCCAACCCCGACTTCCAGATGGTCATTGGCTTCCATGGG | 7395 |
| | | \|\| \|\| \|\|\|\|\|\|\|\|\|\|\|\|\|\|\|\|\| \|\|\|\|\| \|\| \|\|\|\|\|\| \|\|\|\| \|\|\|\|\| \|\|\|\|\|\|\|\| | |
| SBJCT: | 7264 | TACGGAGAGATTTATTATGACTCAAACCCTGATTTCCAGCTGGTTATTGGGTTCCATGGA | 7323 |
| QUERY: | 7396 | GGACTCTATGACCCCCTGACCAAGCTGGTCCACTTCACTCAGCGTGATTATGATGTGCTG | 7455 |
| | | \|\| \|\| \|\|\|\|\| \|\| \| \|\|\|\|\| \|\| \|\|\|\|\| \|\| \|\| \|\| \| \|\| \|\| \|\|\|\|\| \|\| | |
| SBJCT: | 7324 | GGGCTGTATGATCCTTTAACCAAACTCGTCCATTTTACCCAAAGGGACTACGATGTCCTT | 7383 |
| QUERY: | 7456 | GCAGGACGATGGACCTCCCCAGACTATACCATGTGGAAAAACGTGGGCAAGGAGCCGGCC | 7515 |
| | | \|\| \|\|\|\|\| \|\|\|\|\| \|\| \|\| \|\| \|\| \|\| \|\|\|\|\|\|\|\|\|\|\|\| \| \|\| \| \|\| \|\| \|\| | |
| SBJCT: | 7384 | GCTGGACGCTGGACATCTCCTGATTACACAATGTGGAAAAACATTGGTAGAGAACCTGCT | 7443 |
| QUERY: | 7516 | CCCTTTAACCTGTATATGTTCAAGAGCAACAATCCTCTCAGCAGTGAGCTAGATTTGAAG | 7575 |
| | | \|\|\|\|\| \|\| \|\|\|\|\| \|\|\|\|\|\|\|\|\|\|\| \|\|\|\|\| \|\|\|\|\|\|\|\|\|\| \|\|\| \|\| \|\|\| \| \|\|\| | |
| SBJCT: | 7444 | CCCTTCAATCTGTACATGTTCAAGAGTAACAACCCTCTCAGCAATGAACTGGATCTAAAG | 7503 |
| QUERY: | 7576 | AACTACGTGACAGATGTGAAAAGCTGGCTTGTGATGTTTGGATTTCAGCTTAGCAACATC | 7635 |
| | | \|\| \|\| \|\| \|\|\|\|\|\|\|\| \|\|\|\|\|\|\|\|\|\|\| \|\|\|\|\|\|\|\| \|\|\|\|\|\|\|\|\|\|\|\|\|\|\|\|\|\|\|\| | |
| SBJCT: | 7504 | AATTATGTAACAGATGTCAAAAGCTGGCTGGTGATGTTCGGATTTCAGCTTAGCAACATT | 7563 |
| QUERY: | 7636 | ATTCCTGGCTTCCCGAGAGCCAAAATGTATTTCGTGCCTCCTCCCTATGAATTGTCAGAG | 7695 |
| | | \|\|\|\|\|\|\|\|\|\|\|\|\|\| \|\|\|\|\| \|\|\|\|\|\|\|\| \|\| \|\|\| \| \|\|\|\|\| \|\| \|\| \|\| \|\| \| \|\|\| | |
| SBJCT: | 7564 | ATTCCTGGCTTCCCTAGAGCAAAAATGTACTTTGTGTCACCTCCATACGAGCTGACTGAG | 7623 |
| QUERY: | 7696 | AGTCAAGCAAGTGAGAATGGACAGCTCATTACAGGTGTCCAACAGACAACAGAGAGACAT | 7755 |
| | | \|\|\|\|\|\|\|\| \|\|\|\| \|\|\|\|\|\|\|\|\|\|\| \|\|\|\|\|\|\|\| \|\|\|\|\| \|\|\|\|\|\|\|\|\|\|\| \|\|\|\|\| | |
| SBJCT: | 7624 | AGTCAAGCGTGTGAAAATGGACAGCTAATTACAGGAGTCCAGCAGACAACAGAAAGACAC | 7683 |
| QUERY: | 7756 | AACCAGGCCTTCATGGCTCTGGAAGGACAGGTCATTACTAAAAAGCTCCACGCCAGCATC | 7815 |
| | | \|\| \|\| \|\| \|\|\|\|\|\|\|\|\|\|\| \|\| \|\|\|\|\|\|\|\|\|\|\| \|\|\|\|\|\| \| \|\| \|\|\|\|\| \|\| | |
| SBJCT: | 7684 | AATCAAGCTTTCATGGCTCTTGAGGGACAGGTCATATCTAAAAGATTACATGCCAGTATT | 7743 |
| QUERY: | 7816 | CGAGAGAAAGCAGGTCACTGGTTTCCCACCACCACGCCCATCATTGGCAAAGGCATCATG | 7875 |
| | | \|\|\|\| \|\|\|\|\|\|\|\| \|\|\|\|\|\|\|\|\|\|\| \|\| \| \|\|\| \|\| \|\| \|\|\|\|\| \|\|\|\|\| \|\|\|\|\|\| | |
| SBJCT: | 7744 | AGAGAAAAAGCAGGCCACTGGTTTGCAACAAGCACTCCTATTATTGGGAAAGGAATCATG | 7803 |
| QUERY: | 7876 | TTTGC 7880 | |
| | | \|\|\|\|\| | |
| SBJCT: | 7804 | TTTGC 7808 | |

SCORE = 339 BITS (171), EXPECT = 2E − 89
IDENTITIES = 429/515 (83%)
STRAND = PLUS/PLUS

| | | | |
|---|---|---|---|
| QUERY: | 7967 | ACTACCTGGACAAGATGCACTACAGCATCGAGGGCAAGGACACCCACTACTTTGTGAAGA | 8026 |
| | | \|\|\|\|\|\|\|\|\|\| \|\| \|\|\|\|\|\|\|\|\|\|\|\|\|\|\|\|\|\|\|\| \|\|\|\|\| \|\| \|\|\|\|\|\|\|\|\|\|\| \|\|\|\| | |
| SBJCT: | 7895 | ACTACCTGGAAAAAATGCACTACAGCATCGAGGGGAAGGATACTCACTACTTTGTCAAGA | 7954 |
| QUERY: | 8027 | TTGGCTCAGCCGATGGCGACCTGGTCACACTAGGCACCACCATCGGCCGCAAGGTGCTAG | 8086 |
| | | \| \|\|\|\|\|\|\|\|\|\|\|\| \|\|\|\|\|\|\| \|\|\|\|\| \|\| \| \| \|\|\|\| \|\|\| \| \|\|\|\|\| \|\| \| | |
| SBJCT: | 7955 | TAGGCTCAGCCGATAGCGACCTCGTCACCCTCGCGATGACCAGCGGGAGGAAGGTCCTGG | 8014 |
| QUERY: | 8087 | AGAGCGGGGTGAACGTGACCGTGTCCCAGCCCACGCTGCTGGTCAACGGCAGGACTCGAA | 8146 |
| | | \| \|\|\|\|\| \|\| \|\|\|\|\|\|\|\|\|\|\| \|\|\|\|\|\|\|\| \|\| \|\| \|\| \|\|\|\|\|\|\| \|\|\|\|\|\|\|\|\| | |
| SBJCT: | 8015 | ACAGCGGAGTAAACGTGACCGTCTCCCAGCCAACCCTCCTTATCAACGGAAGGACTCGAC | 8074 |
| QUERY: | 8147 | GGTTCACGAACATTGAGTTCCAGTACTCCACGCTGCTGCTCAGCATCCGCTATGGCCTCA | 8206 |
| | | \|\|\|\|\|\|\| \|\|\|\|\| \|\|\|\|\| \|\|\|\|\| \|\|\|\|\| \|\|\|\|\|\| \|\|\| \|\|\|\|\|\|\|\|\| \|\| \|\|\|\| | |
| SBJCT: | 8075 | GGTTCACAAACATCGAGTTTCAGTATTCCACCCTGCTGATCAACATCCGCTACGGGCTCA | 8134 |
| QUERY: | 8207 | CCCCCGACACCCTGGACGAAGAGAAGGCCCGCGTCCTGGACCAGGCGAGACAGAGGGCCC | 8266 |
| | | \|\| \|\|\|\|\|\|\| \|\|\|\|\| \|\| \|\|\|\|\|\|\|\| \|\| \|\| \|\| \|\|\|\|\|\|\|\| \| \|\|\| \| \|\|\|\| | |
| SBJCT: | 8135 | CCGCCGACACGCTGGATGAGGAGAAGGCACGAGTGCTAGACCAGGCTCGGCAGCGAGCCC | 8194 |
| QUERY: | 8267 | TGGGCACGGCCTGGGCCAAGGAGCAGCAGAAAGCCAGGGACGGGAGAGAGGGGAGCCGCC | 8326 |
| | | \|\|\|\| \|\|\|\|\|\|\|\|\|\|\|\|\| \|\|\|\|\|\|\|\|\|\|\| \|\| \|\|\|\| \|\| \| \|\|\|\|\| \|\|\|\|\|\| | |
| SBJCT: | 8195 | TGGGGTCGGCCTGGGCCAAAGAGCAGCAGAAGGCACGGGATGGCCGCGAGGGCAGCCGCG | 8254 |
| QUERY: | 8327 | TGTGGACTGAGGGCGAGAAGCAGCAGCTTCTGAGCACCGGCGCGTGCAAGGGTACGAGG | 8386 |
| | | \| \|\|\|\|\| \|\| \|\| \|\|\|\|\|\|\|\| \|\|\|\|\|\|\|\|\|\| \|\|\| \|\| \| \|\| \|\|\|\|\| \|\|\|\|\|\|\| | |
| SBJCT: | 8255 | TATGACAGACGGAGAGAAGCAACAGCTTCTGAACACGGGAAGGGTTCAAGGTTACGAGG | 8314 |
| QUERY: | 8387 | GATATTACGTGCTTCCGTGGAGCAGTACCCAGAGCTAGCAGACAGTAGCAGCAACATCC | 8446 |
| | | \|\|\|\|\|\|\| \|\| \| \|\| \|\|\|\|\|\|\|\| \|\|\|\|\|\|\|\|\|\|\| \|\|\|\|\|\|\|\|\|\|\|\|\|\|\|\|\|\|\|\|\|\| | |
| SBJCT: | 8315 | GATATTATGTCTTGCCTGTGGAGCAGTACCCAGAGCTAGCAGACAGTAGCAGCAACATCC | 8374 |
| QUERY: | 8447 | AGTTTTTAAGACAGAATGAGATGGGAAAGAGGTAA 8481 | |
| | | \|\|\|\|\|\|\|\|\|\|\|\|\|\|\|\|\|\|\| \|\|\|\|\|\|\|\|\|\|\|\|\|\|\| | |
| SBJCT: | 8375 | ACTTTTTAAGACAGAATGAAATGGGAAAGAGGTAA 8409 | |

SCORE = 323 BITS (163), EXPECT = 1E − 84
IDENTITIES = 397/475 (83%)
STRAND = PLUS/PLUS

| | | | |
|---|---|---|---|
| QUERY: | 299 | GACACCGCTCTTTGACCAGAGGACGCTGTGGCAAAGAGTGTCGCTACACAAGCTCCTCTC | 358 |
| | | \|\|\|\|\|\|\|\|\|\|\|\|\|\|\|\| \|\|\|\|\| \|\| \|\| \|\| \|\| \|\|\|\|\|\|\|\|\|\|\| \|\| \|\| \|\| \|\| \| | |
| SBJCT: | 20 | GACACCCCTCTTTGACGAGAGGCCGGTGCGGGAAGGAGTGTCGCTATACTAGTTCTTCAC | 79 |
| QUERY: | 359 | TGGACAGTGAGGACTGCCGGGTGCCCACACAGAAATCCTACAGCTCCAGTGAGACTCTGA | 418 |
| | | \| \|\|\|\|\|\|\|\| \|\|\|\|\|\| \| \|\| \|\| \| \|\|\|\|\| \|\|\|\|\|\|\|\|\|\|\|\|\|\|\|\|\|\|\|\| \|\|\|\| | |
| SBJCT: | 80 | TCGACAGTGAAGACTGCAGAGTACCAGCTCAGAAGTCCTACAGCTCCAGTGAGACCCTGA | 139 |
| QUERY: | 419 | AGGCCTATGACCATGACAGCAGGATGCACTATGGAAACCGAGTCACAGACCTCATCCACC | 478 |
| | | \| \|\| \|\|\|\| \|\|\|\|\|\|\|\| \|\|\|\|\|\|\|\|\|\|\| \|\|\|\|\| \|\|\|\|\| \|\|\|\|\|\|\| \| \|\|\| | |
| SBJCT: | 140 | AAGCATATGGCCATGACACGAGGATGCACTACGGAAATCCAGTTTCAGACCTGGTTCACA | 199 |
| QUERY: | 479 | GGGAGTCAGATGAGTTTCCTAGACAAGGAACCAACTTCACCCTTGCCGAACTGGGCATCT | 538 |
| | | \|\|\|\|\|\|\| \|\|\|\|\|\|\|\|\|\|\| \|\| \|\|\|\|\|\|\|\| \|\|\|\|\|\|\|\|\|\|\|\|\|\| \|\|\|\|\|\|\|\| \|\|\|\| | |
| SBJCT: | 200 | GGGAGTCGGATGAGTTTCCAAGGCAAGGAACGAACTTCACCCTTGCAGAACTGGGAATCT | 259 |
| QUERY: | 539 | GTGAGCCCTCCCCACACCGAAGCCGCTACTGCTCCGACATGGGGATCCTTCACCAGGGCT | 598 |
| | | \|\|\|\|\|\|\|\|\|\| \|\| \|\| \|\|\|\|\| \|\|\|\|\|\|\|\|\|\|\| \|\|\|\|\| \|\| \|\| \|\| \|\| \|\| \|\|\|\| | |
| SBJCT: | 260 | GTGAGCCCTCTCCCCATCGAAGTGGCTACTGCTCGGACATAGGAATACTCCATCAAGGCT | 319 |
| QUERY: | 599 | ACTCCCTTAGCACACGGTCTGACGCCGACTCCGACACCGAGGGAGGGATGTCTCCAGAAC | 658 |
| | | \| \|\|\| \| \|\|\|\|\| \|\| \|\|\|\|\| \|\| \|\|\|\|\| \|\|\|\|\| \|\|\|\|\| \|\|\|\|\|\|\|\|\|\|\|\|\|\| \| | |
| SBJCT: | 320 | ATTCCTTGAGCACTGGCTCTGATGCTGACTCAGACACGGAGGGCGGGATGTCTCCAGAGC | 379 |
| QUERY: | 659 | ACGCCATCAGACTGTGGGGCAGAGGGATAAAATCCAGGCGCAGTTCCGGCCTGTCCAGTC | 718 |
| | | \|\|\|\| \|\|\|\|\| \|\|\|\|\|\|\|\| \|\|\|\|\|\|\|\| \|\|\|\|\|\|\|\| \|\| \|\|\|\|\| \|\|\|\|\|\|\|\| \|\|\|\| | |
| SBJCT: | 380 | ACGCGATCAGGCTGTGGGGAAGAGGGATCAAATCCAGCCGAAGTTCTGGCCTGTCAAGTC | 439 |
| QUERY: | 719 | GTGAAAACTCGGCCCTTACCCTGACTGACTCTGACAACGAAAACAAATCAGATGA 773 | |
| | | \|\|\|\|\|\|\|\|\|\|\|\|\| \|\| \|\| \|\| \|\|\|\|\|\|\|\| \|\|\|\|\| \|\| \|\|\|\|\| \|\|\|\|\|\|\|\| | |
| SBJCT: | 440 | GTGAAAACTCGGCTCTCACGCTCACTGACTCCGACAATGAGAACAAGTCAGATGA 494 | |

The full FCTR3a amino acid sequence also has 342 of 383 amino acid residues (89%) identical to, and 342 of 383 residues (89%) positive with, the 276 amino acid residue Odd Oz/ten-m homolog 2 (Drosophila) (GenBank Acc: NP_{—}035986.2) (SEQ ID NO:68) (Table 3P).

TABLE 3P
BLASTP of FCTR3a against Odd Oz/ten-m homolog 2-(SEQ ID NO:68)
>GI|7657415|REF|NP 035986.2| ODD OZ/TEN-M HOMOLOG 2 (DROSOPHILA); ODD OZ/TEN-
M HOMOLOG 3

(DROSOPHILA) [MUS MUSCULUS]
GI|4760778|DBJ|BAA77397.1| (AB025411) TEN-M2 [MUS MUSCULUS]
LENGTH = 2764
SCORE = 495 BITS (1274), EXPECT = E − 139
IDENTITIES = 342/383 (89%), POSITIVES = 342/383 (89%), GAPS = 41/383 (10%)

| | | | |
|---|---|---|---|
| QUERY: | 37 | HNPPPVSCQMPLLDSNTSHQIMDTNPDEEFSPNSYLLRACSGPQQASSSGPPNHIHSQSTL | 96 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 189 | HNPPPVSCQMPLLDSNTSHQIMDTNPDEEFSPNSYLLRACSGPQQASSSGPPNHHSQSTL | 248 |
| QUERY: | 97 | RPPLPPPHNHTLSHHHSSANSLNRNSLTNRRSQIHAPAPAPNDLATTPESVQLQDSWVLN | 156 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 249 | RPPLPPPHNHTLSHHHSSANSLNRNSLTNRRSQIHAPAPAPNDLATTPESVQLQDSWVLN | 308 |
| QUERY: | 157 | SNVPLETRHFLFKTSSGSTPLFSSSSPGYPLTSGTVYTPPPRLLPRNTFSRKAFLKKPS | 216 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 309 | SNVPLETRHFLFKTSSGSTPLFSSSSPGYPLTSGTVYTPPPRLLPPNTFSRKAFKLKKPS | 368 |
| QUERY: | 217 | KYCSWKCAALSAIAAALLLAILLAYFI--------------------------------- | 243 |
| | | \|\|\|\|\|\|\|\|\|\|\|\|\|\|\|\|\|\|\|\|\|\|\|\|\|\|\| | |
| SBJCT: | 369 | KYCSWKCAALSAIAAALLLAILLAYFIAMHLLGLNWQLQPADGHTFNNGVRTGLPGNDDV | 428 |
| QUERY: | 244 | --------VPWSLKNSSIDSGEAEVGRRVTQEVPPGVFWRSQIHISQPQFLKFNISLGKD | 295 |
| | | \|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 429 | ATVPSGGKVPWSLKNSSIDSGEAEVGRRVTQEVPPGVFWRSQIHISQPQFLKFNISLGKD | 488 |
| QUERY: | 296 | ALFGVYIRRGLPPSHAQYDFMERLDGKEKWSVVESPRERRSIQTLVQNEAVFVQYLDVGL | 355 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 489 | ALFGVYIRRGLPPSHAQYDFMERLDGKEKWSVVESPRERRSIQTLVQNEAVFVQYLDVGL | 548 |
| QUERY: | 356 | WHLAFYNDGKDKEMVSFNTVVLD 378 | |
| | | \|\|\|\|\|\|\|\|\|\|\|\|\|\|\|\|\|\|\|\|\|\|\| | |
| SBJCT: | 549 | WHLAFYNDGKDKEMVSFNTVVLD 571 | |

The full FCTR3b amino acid sequence has 2442 of 2802 amino acid residues (87%) identical to, and 2532 of 2802 residues (90%/O) positive with, the 2802 amino acid residue teneurin-2 [Gallus gallus] (GenBank Acc: AJ279031) (SEQ ID NO:69) (Table 3Q).

TABLE 3Q
BLASTP of FCTR3a against Teneurin-2-(SEQ ID NO:69

>GI|10241574|EMB|CAC09416.1| (AJ279031) TENEURIN-2 [GALLUS GALLUS]
LENGTH = 2802
SCORE = 4853 BITS (12589), EXPECT = 0.0
IDENTITIES = 2510/2802 (87%), POSITIVES = 2600/2802 (90%) , GAPS = 69/2802 (2%)

| | | | |
|---|---|---|---|
| QUERY: | 1 | MDVRDRRHRSLTRGRCGKECRYTSSSLDSEDCRVPTQKSYSSSETLKAYDEDSRMRYGNR | 60 |
| | | \|\|+\|\|\|\|\|\|\|\|\|\|\|\|\|\|\|\|\|\|\|\|\|\|\|\|\|\|\|\|\|\|\|\| \|\|\|\|\|\|\|\|\|\|\|\|\| \|\| \|\|\|\|\|\|\| | |
| SBJCT: | 1 | MDIKDRRHRSLTRGRCGKECRYTSSSLDSEDCRVPAQKSYSSSETLKAYGEDTRMHYGNR | 60 |
| QUERY: | 61 | VTDLIHRESDEFPRQGTNFTLLGICEPSPERSGYCSDMGILEQGYSLSTGSDADSDTE | 120 |
| | | \|+\|\|+\|\|\|\|\|\|\|\|\|\|\|\|\|\|\|\|\|\|\|\|\|\|\|\|\|\|\|\|\|\|\|\|\|\|+\|\|\|\|\|\|\|\|\|\|\|\|\|\|\|\|\|\|\|\| | |
| SBJCT: | 61 | VSDLVHRESDEFPRQGTNFTLAELGICEPSPHRSGYCSDIGILEQGYSLSTGSDADSDTE | 120 |
| QUERY: | 121 | GGMSPEAIRLWGRGIKSRRSSGLSSRENSALTLTDSDNENKSDDENG------------ | 168 |
| | | \|\|\|\|\|\|\|\|\|\|\|\|\|\|\|\|\|\| \|\|\|\|\|\|\|\|\|\|\|\|\|\|\|\|\|\|\|\|\|\|\|\|\| \|\| | |
| SBJCT: | 121 | GGMSPEHAIRLWGRGIKSSRSSGLSSRENSALTLTDSDNENKSDEENDFETHLSEKLKDR | 180 |
| QUERY: | 169 | ----------------RPIPPTSSPSLLPSAQLPSSHNPPPVSCQMPLLDSNTSEQIMDT | 212 |
| | | \|\|\|\|\|\|\|\| \|\|\|\|\|\|\|\|\|\|\|\|\|\|\|\|\|\|\|\|\|\|\|\|\|\|\|\|\|\|\| | |
| SBJCT: | 181 | QTSWQQLAETKNSLIRRPIPPTSSSSLLPSAQLPSSENPPPVSCQMPLLDSNTSHQIMDT | 240 |
| QUERY: | 213 | NPDEEFSPNSYLLRACSGPQQASSSGPPNHHSQSTLRPPLPPPHWHTLSHHHSSANSLNR | 272 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 241 | NPDEEFSPNSYLLRACSGPQQASSSGPSNESQSTLRPPLPPPHNHSLSHHHSSANSLNR | 300 |
| QUERY: | 273 | XXXXXXXXQIHAPAPAPNDLATTPESVQLQDSWVLNSNVPLETREFLFKXXXXXXXXXXX | 332 |
| | | \|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 301 | NSLTNRRNQIHAPAPAPNDLATTPESVQLQDSWVLNSNVPLETRHFLFKTSSGTTPLFSS | 360 |
| QUERY: | 333 | XXXXYPLTSGTVYTPPPRLLPRNTFSRXAFKLKKPSKYCSWKCXKCXXXXXXXXXXXXXX | 392 |
| | | \|\|\|\|\|\|\|\|\|\|\|\|\|\|\|\|\|\|\|\|\|\|\| \|\|\|\|\|\|\|\|\|\|\|\|\|\|\| | |
| SBJCT: | 361 | SSPGYPLTSGTVYTPPPRLLPRNTFSRNAFKLKKPSKYCSWKCAALSAIAAAVLLAILLA | 420 |
| QUERY: | 393 | YFIV-----------------------------------------PWSLKNSSIDSGEAE | 411 |
| | | \|\|\| \|\| +\|\|\|\|\|\|\|\| \| | |
| SBJCT: | 421 | YFIAHLLGLNWQLQPADGETFSNGLRPGAAGAEDGAAAPPAGRGPWVTRNSSIDSGETE | 480 |
| QUERY: | 412 | VGRRVTQEVPPGVFWRSQIEISQPQFLKFNISLGKDALFGVYIRRGLPPSHAQYDFMERL | 471 |
| | | \|\|\|+\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 481 | VGRKVTQEVPPGVFWRSQIEISQPQFLKFNISLGKDALFGVYIRRGLPPSHAQYDFMERL | 540 |
| QUERY: | 472 | DGKEKWSVVESPRERRSIQTLVQNEAVFVQYLDVGLWHLAFYNDGKDKEMVSFNTVVLDS | 531 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|+\|\|\|+\|\|+\|\|\| | |
| SBJCT: | 541 | DGKERWSVVESPRERRSIQTLVQNEAVFVQYLDVGLWHLAFYNDGKDKEVVSFSTVILDS | 600 |
| QUERY: | 532 | VQDCPRNCHGNGECVSGVCHCFPGFLGADCAAACPVLCSGNGQYSKGTCQCYSGWKGAE | 591 |
| | | \|\|\|\|\|\|\|\|\|\|\|\|\|\|\|\|\|\|\|\|\|\|\|\|\| \|\|\|\|\|\|\|\|\|\|\|\|\|\|\|\|\|\|\|\|\|\|\| \|\|\|\|\|\|\| \| | |
| SBJCT: | 601 | VQDCPRNCHGNGECVSGVCHCFPGFHGADCAKAACPVLCSGNGQYSKGTCLCYSGWKGPE | 660 |
| QUERY: | 592 | CDVPMNQCIDPSCGGHGSCIDGNCVCSAGYKGEHCEEVDCLDPTCSSHGVNGECLCSP | 651 |
| | | \|\|\|\|++\|\|\|\|\|\|\|\|\|\|\|\|\|+\|\|\|\|\|\| \|\|\|\|\|+\|\|\|\|\|\|\|\|\|\|\|\|+\|\|\|\|\|\|\|\|\|\|\|\|\| | |
| SBJCT: | 661 | CDVPISQCIDPSCGGHGSCIEGNCVCSIGYKGENCEEVDCLDPTCSNHGVCVNGECLCSP | 720 |
| QUERY: | 652 | GWGNCELRVQCPDQCSGHGTYLPDTGLCSCDPNMGPDCSVEVCSVDCGTHGVCIGG | 711 |
| | | \|\|\|\|+\|\|\|\| \| \|\|\|\|\|\|\|\|\|\|\|\|\| \|\|\|\|\|\|\|\|\|\|\|\|\|\|\|\|\|\|\|\|\|\|\|\|\|\|\|\|\|\|\|\|\|\| | |
| SBJCT: | 721 | GWGGINCELPRAQCPDQCSGEGTYLSDTGLCSCDPMGPDCSVEVCSVDCGTHGVCIGG | 780 |
| QUERY: | 712 | ACRCEEGWTGAACDQRVCHPRCIEHGTCKDGKCECREGWNGEHCTIGRQTAGTETDGCPD | 771 |
| | | \|\|\|\|\|\|\|\|\|\| \|\|\|\|\|\|\|\|\|\|\| \|\|\|\|\|\|\|\|\|\|\|\|\|\|\|\|\|\|\|\|\|\|\|\|\|\|\| \|\|\|\|\|\|\|\|\| | |
| SBJCT: | 781 | ACRCEEGWTGVACDQRVCHPRCTEHGTCKDKCECREGWNGEECTIGRQTTGTETDGCPD | 840 |
| QUERY: | 772 | LCNGNGRCTLGQNSWQCVCQTGWRGPGCNVAMETSCADNKDNEGDGLVDCLDPDCCLQSA | 831 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| \|\|\|\|\|\|\| | |
| SBJCT: | 841 | LCNGNGRCTLGQNSWQCVCQTGWRGPGCNVAMETSCADNKDNEGDGLVDCLVPDCCLQST | 900 |
| QUERY: | 832 | CQNSLLCRGSRDPLDIIQQGQTDWPAVKSFYDRIKLLAGKDSTHIIPGENPFNSSLVSLI | 891 |
| | | \|\|\|\|\|\|\|\|\|\|\|\|\|\|\|\|\|\|\| + \|\|\|\|\|\|\|\|\|\|\|\|\| \|\|\|\|\|\|\|\|\|\|\|\|\|\|\|\|\|\|\|\|\|\| | |
| SBJCT: | 901 | CQNSLLCRGSRDPLDIIQQSHSGSPAVKSFYDRIKLLVGKDSTHIIPGENPFNSSLVSLI | 960 |
| QUERY: | 892 | RGQVVTTDGTPLVGVSFVKYPKYGYTITRQDGTFDLIANGGASLTLHFERAPFMSQER | 951 |
| | | \|\|\|\|\|\|\|\|\|\|\|\|\|\|\|\|\|\|\|\|\|\|\|\|\|\|\|\|\|\|\|\|\|\| \|\|\|+\|\|\|\|+\|\|\|\|\|\|\|\|\|\|\|\|\|\|\|\| | |
| SBJCT: | 961 | RGQVVTTDGTPLVGVNVSFVKYPKYGYTITRQDGMFDLVANGGSSLTLHFERAPFMSQER | 1020 |
| QUERY: | 952 | TVWLPWNSFYAMDTLVMKTEENSIPSCDLSGFVRPDPIIISSPLSTFFSAAPGQNPIVPE | 1011 |
| | | \|\|\|\|\|\|\|\|\|\|\|\|\|\|\|\|\|\|\|\|\|\|\|\|\|\|\|\|\|\|\|\|\|\|\|\|\|+\|\|\|\|\|\|\|\|\|\|\| \|\|\|+\|\|\|\|\|\| | |
| SBJCT: | 1021 | TVWLPWNSFYAMDTLVMKTEENSIPSCDLSGFVRPDPVIISSPLSTFFSDAPGRNPIVPE | 1080 |
| QUERY: | 1012 | TQVLHEEIELPGSNVKLRYLSSRTAGYKSLLKITMTQSTVPLNLIRVHLMVAVEGHLFQK | 1071 |
| | | \|\|\|\|\|\|\|\|\|+\|\|\|++\|\| \|\|\|\|\|\|\|\|\|\|\|\|\|\|\| \|\|\|\| \|\|\|\|\|\|+\|\|\|\|\|\|\|\|\|\|\|\|\|\| | |
| SBJCT: | 1081 | TQVHEEIEVPGSSIKLIYLSSRTAGYRSLLKIIMTQSLVPLNLIKVHLMVAVEGHLFQK | 1140 |
| QUERY: | 1072 | SFQASPNASTFIWDKTDAYGQRVYGLSDAVVSVGFEYETCPSLILWEKRTALLQGFELD | 1131 |
| | | \|\| \|\|\|\|\|\| \|\|\|\|\|\|\|\|\|\|\|\|+\|\|\|\|\|\|\|\|\|\|\|\|\|\|\|\|\|\|\|\|\|\|\|\|\|\|\|\|\|\|\|\|\|\|\|\|\| | |
| SBJCT: | 1141 | SFLASPNLAYTFIWDKTDAYGQKVYGLSDAVVSVGFEYETCPSLILWEKRTALLQGFELD | 1200 |
| QUERY: | 1132 | PSNLGGWSLDKHHILNVKSGILEKGTGENQFLTQQPAIITSIMGNGRRRSISCPSCNGLA | 1191 |
| | | \|\|\|\|\|\|\|\|\|\|\|\|\|+\|\|\|\|\|\|\|\|\|\|\| \|\|\|\|\|\|\|\|\|\|\|+\|\|\|\|\|\|\|\|\|\|\|\|\|\|\|\|\|\|\|\|\|\| | |
| SBJCT: | 1201 | PSNLGGWSLDKEHVLNVKSGILHKGNGENQFLTQQPAVITSIMGNGRRRSISCPSCNGLA | 1260 |
| QUERY: | 1192 | EGNKLLAPVALAVGIDGSLYVGDFNYIRRIFPSRNVTSILELRNKEFKHSNNPAHKYYLA | 1251 |
| | | \|\|\|\|\|\|\|\|\|\|\|\|\|\|\|\|\|\|\|+\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 1261 | EGNKLLAPVALAVGIDGSLFVGDFNYIRRIFPSRNVTSILELRNKEFKHSNNPAHKYYLA | 1320 |
| QUERY: | 1252 | VDPVSGSLYVSDTNSRRIYRVKSLSGTKDLAGNSEVVAGTGEQCLPFDEARCGDGGKAID | 1311 |
| | | \|\|\|\|\|\|\|\|\|\|\|\|\|\|\|\|\|\|\|+\|\|\|\|+\|\|\|\|\|\|\|\|\|\|\|\|\|\|\|\|\|\|\|\|\|\|\|\|\|\|\|\|\|\|\|\|\|+\| | |
| SBJCT: | 1321 | VDPVSGSLYVSDTNSRRIYKVKSLTGTKDLAGNSEVVAGTGEQCLPFDEARCGDGGKAVD | 1380 |
| QUERY: | 1312 | ATLMSPRGIAVDKNGLMYFVDATMIRKVDQNGIISTLLGSNDLTAVRPLSCDSSMDVAQV | 1371 |
| | | \|\|\|\|\|\|\|\|\|\|\|\|\| \|\|\|\|\|\|\|\|\|\|\|\|\|\|\|\|\|\|\|\|\|\|\|\|\|\|\|\|\|\|\|\|\|\|\|\|\|\|\|\|\|\|\|+\|\| | |
| SBJCT: | 1381 | ATLMSPRGIAVDKYGLMYFVDATMIRKVDQNGIISTLLGSNDLTAVRPLSCDSSMDVSQV | 1440 |
| QUERY: | 1372 | RLEWPTDLAVNPMDNSLYVLENNVILRITENHQVSIIAGRPNHCQVPGIDYSLSKXXXXX | 1431 |
| | | \|\|\|\|\|\|\|\|\|\|+\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 1441 | RLEWPTDLAVDPMDNSLYVLENNVILRITENHQVSIIAGRPMHCQVPGIDYSLSKLAIHS | 1500 |
| QUERY: | 1432 | XXXXXXXXXXXXTGVLYITETDEKKINRLRQVTTNGEICLLAGAASXXXXXXXXXXXXYS | 1491 |
| | | \|\|\|\|\|\|+\|\|\|\|\|\|\|\|\|\|\|\|\|\|\|\|\|\|\|\|\|\|\|\|\|\|\| \|\| | |
| SBJCT: | 1501 | ALESASAIAISHTGVLYISETDEKKIRLRQVTTNGEICLLAGAASDCDCKNDVNCNCYS | 1560 |
| QUERY: | 1492 | GDDAYATDAILNSPSSLAVAPDGTIYIADLGNIRIRAVSKNKPVLNAFNQYEAASPGEQE | 1551 |
| | | \|\|\| \|\|\|\|\|\|\|\|\|\|\|\|\|\|\|\|\|\|\|\|\|\|\|\|\|\|\|\|\|\|\|\|\|\|\|\|\|+\|+\|\|+\|\|\|\|\|\|\|\|\|\|\|\|\| | |
| SBJCT: | 1561 | GDDGYATDAILNSPSSLAVAPDGTIYIADLGNIRIRAVSKNRPILNSFNQYEAASPGEQE | 1620 |
| QUERY: | 1552 | LYVFNADGIHQYTVSLVTGEYLYNFTYSTDNDVTELIDNNGNSLKIRRDSSGMPRHLLMP | 1611 |
| | | \|\|\|\|\|\|\|\|\|\|\|\|\|+\|\|\|\|\|\|\|\|\|\|\|\|\|\|+\|\|\|\|\|\|++\|+\|\|\|\|\|\|+\|\|\|+\|\|\|\|\|\|\|\|\|\| | |
| SBJCT: | 1621 | LYVFNADGIHQYTLSLVTGEYLYNFTYSSDNDVTEVMDSNGNSLKVRRDASGMPRHLLMP | 1680 |
| QUERY: | 1612 | DNQIITLTVGTNGGLKVVSTQNLELGLMTYDGNTGLLATKSDETGWTTFYDYDHEGRLTN | 1671 |
| | | \|\|\|\|+\|\| \|\|\|\|\|\|\|\|+\|\|\|\| \|\|\|\|\|\|\|\|+\|\|+\|\|\|\|\|\|\|\|\|\|\|\|\|\|\|\|\|\|\|\|\|\|\|\|\|\| | |
| SBJCT: | 1681 | DNQIVTLAVGTNGGLKLVSTQTLELGLMTYNGNSGLLATKSDETGWTTFYDYDHEGRLTN | 1740 |
| QUERY: | 1672 | VTRPTGVVTSLHREMEKSITIDIENSNRDDDVTVITNLSSVEASYTVVQDQVRNSYQLCN | 1731 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 1741 | VTRPTGVVSLHREMEKSITIDIENSNRDDDVTVITNLSSVEASYTVVQDQVRNSYQLCN | 1800 |
| QUERY: | 1732 | NGTLRVMYANGMGISFHSEPHVLAGTITPTIGRCNISLPMENGLNSIEWRLRKEQIKGKV | 1791 |
| | | \|\|\|\|\|\|\|\|\|\|\|\| \|\|\|\|\|\|\|\|\|\|\|\|\|+\|\|\|\|\|\|\|\|\|\|\|\|\|\|\|\|\|\|\|\|\|\|\|\|\|\|\|\|\|\|\|\|\| | |
| SBJCT: | 1801 | NGTLRVMYANGMSISFHSEPHVLAGTVTPTIGRCNISLPMENGLNSIEWRLRKEQIKGKV | 1860 |
| QUERY: | 1792 | TIFGRKLRVHGRNLLSIDYDRNIRTEKIYDDHKFTLRIIYDQVGRPFLWLPSSGLAAVN | 1851 |
| | | \|+\|\|\|\|\|\|\|\|\|\|\|\|\|\|\|\|\|\|\|\|\|\|\|\|\|\|\|\|\|\|\|\|\|\|\|\|\|\|\|\|\|+\|\|\|\|\|\|\|\|\|\|\|\|\|\|\|\| | |
| SBJCT: | 1861 | TVFGRKLRVHGRNLLSIDYDRNIRTEKIYDDHRKFTLRIIYDQLGRPFLWLPSSGLAAVN | 1920 |
| QUERY: | 1852 | VSYFFNGRLAGLQRGAMSERTDIDKQGRIVSRMFADGKVWSYSYLDKSMVLLLQSQRQYI | 1911 |
| | | \|\|\|\|\|\|\|\|\|\|\|\|\|\|\|\|\|\|\|\|\|\|\|\|\|\|\|\|\|\|+\|\|\|\|\|\|\|\|\|\|\|\|+\|\|+\|\|\|\|\|\|\|\|\|\|\|\|\| | |
| SBJCT: | 1921 | VSYFFNGRLAGLQRGAMSERTDIDKQGRIISRMFADGKVWSYTYLEKSMVLLLQSQRQYI | 1980 |
| QUERY: | 1912 | FEYDSSDRLLAVTMPSVARXSMSTHTSIGYIRNIYNPPESNASVIFDYSDDGRILKTSFL | 1971 |
| | | \|\|\|\|\|\|\|\|\| \|\|\|\|\|\|\|\|\|\|\|\|\|\|\|\|\|+\|\|\|\|\|\|\|\|\|\|\|\|\|\|\|\|\|\|\|\|\|\|\|\|\|\|\|\|\|\|\|\| | |
| SRJCT: | 1981 | FEYDSSDRLHAVTMPSVARHSMSTHTSVGYIRNIYNPPESNASVIFDYSDDGRILKTSFL 2040 | |
| QUERY: | 1972 | GTGRQVFYKYGKLSKLSEIVYDSTAVTFGYDETTGVLKMVNLQSGGFSCTIRYRKIGPLV | 2031 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 2041 | GTGRQVFYKYGKLSKLSEIVYDSTAVTFGYDETTGVLKMVNLQSGGFSCTIRYRKIGPLV | 2100 |
| QUERY: | 2032 | DKQIYRFSEEGMVNARFDYTYHDNSFRIASIKPVISETPLPVDLYRYDEISGKVEHFGKF | 2091 |
| | | \|\|\|\|\|\|\|\|\|\|\|\|\|\|\|\|\|\|\|\|\|\|\|\|\|\|\|\|\|\|\|\|\|+\|\|\|\|\|\|\|\|\|\|\|\|\|\|\|\|\|\|\|\|\|\|\|\|\|\| | |
| SBJCT: | 2101 | DKQIYRFSEEGMVNARFDYTYHDNSFRIASIKPIISETPLPVDLYRYDEISGKVEHFGKF | 2160 |
| QUERY: | 2092 | GVIYYDINQIITTAVMTLSKFDTHGRIEVQYEMFRSLMYWMTVQYDSMGRVIKRELKL | 2151 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| \|\|\|\|\|\| | |
| SBJCT: | 2161 | GVIYYDINQIITTAVMTLSKHFDTHGRIKEVQYEMFRSLMYWMTVQYDSMGRVTKRELKL | 2220 |
| QUERY: | 2152 | GPYANTTKYTYDYDGDGQLQSVAVNDRPTWRYSYDXXXXXXXXXXXXSVRLMPLRYDLRD | 2211 |
| | | \|\|\|\|\|\|\|\|\|\|\|\|\|\|\|\|\|\|\|\|\|\|\|\|\|\|\|\|\|\|\|\|\|\|\| \|\|\|\|\|\|\|\|\|\|\|\|\| | |
| SBJCT: | 2221 | GPYANTTKYTYDYDGDGQLQSVAVNDRPTWRYSYDLNGNLHLLNPGNSVRLMPLRYDLRD | 2280 |
| QUERY: | 2212 | RITRLGDVQYKIDDDGYLCQRGSDIFEYNSKGLLTRAYNKASGWSVQYRYDGVGRRASYK | 2271 |
| | | \|\|\|\|\|\|\|+ \|\|\|\|\|\|\|+\|\|\|\|\|\|\|+\|\|\|\|\|\|\|\|\|\|\|\|\|\|\|\|+\|\|+\|\|\|\|\|\|\|+\|\|\|\|\| \| | |
| SBJCT: | 2281 | RITRLGDIPYKIDDDGFLCQRGSDVFEYNSKGLLTRAYNKANGWNVQYRYDGLGRRASCK | 2340 |
| QUERY: | 2272 | TNLGHHLQYFYSDLHNPTRITHVYNHSNSEITSLYYDLQGHLFAMESSSGEEYYVASDN | 2331 |
| | | \|\|\|\|\|\|\|\|\|\|\|+\|\|\|\|\|\|\|+\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 2341 | TNLGHHLQYFYADLHNPTRVHVYNHSNSEITSLYYDLQGHLFAMESSSGEEYYVASDNT | 2400 |
| QUERY: | 2332 | GTPLAVFSINGLMIKQLQYTAYGEIYYDSNPDFQMVIGFHGGLYDPLTKLVHFTQRDYDV | 2391 |
| | | \|\|\|\|\|\|\|\|\|\|\|\|\|\|\|\|\|\|\|\|\|\|\|\|\|\|\|\|\|\|\|\|\|\|+\|\|\|\|\|\|\|\|\|\|\|\|\|\|\|\|\|\|\|\|\|\|\|\|\| | |
| SBJCT: | 2401 | GTPLAVFSINGLMIKQLQYTAYGEIYYDSNPDFQLVIGFHGGLYDPLTKLVHFTQRDYDV | 2460 |
| QUERY: | 2392 | LAGRWTSPDYTMWKNVGKEPAPFNLYMFKSNNPLSSELDLKYVTDVKSWLVMFGFQLSN | 2451 |
| | | \|\|\|\|\|\|\|\|\|\|\|\|\|\|\|+\|+\|\|\|\|\|\|\|\|\|\|\|\|\|\|\|\|\|+\|\|\|\|\|\|\|\|\|\|\|\|\|\|\|\|\|\|\|\|\|\|\|\| | |
| SBJCT: | 2461 | LAGRWTSPDYTMWKNIGREPAPFNLYMFKSNNPLSNELDLKNYVTDVKSWLVMFGFQLSN | 2520 |
| QUERY: | 2452 | IIPGFPRAKMYFVPPPYELSESQASENGQLITGVQQTTERHNQAFMALEGQVITKKLHAS | 2511 |
| | | \|\|\|\|\|\|\|\|\|\|\|\|\| \|\|\|\|\|+\|\|\|\| \|\|\|\|\|\|\|\|\|\|\|\|\|\|\|\|\|\|\|\|\|\|\|\|\|\|\|\|+\|+\|\|\|\| | |
| SBJCT: | 2521 | IIPGFPRAKMYFVSPPYELTESQACENGQLITGVQQTTERHNQAFMALEGQVISKRLHAS | 2580 |
| QUERY: | 2512 | IREKAGRWFATTTPIIGKGIMFAIKEGRVTTGVSSIASEDSRKVASVLNNAYYLDKMHYS | 2571 |
| | | \|\|\|\|\|\|\|\|\|\|\|+\|\|\|\|\|\|\|\|\|\|\|+\|+\|\|\|\|\|\|+\|\|\|\|++\|\|\|\|+\|\|\|\|\|+\|+\|\|+\|\|\|\|\| | |
| SBJCT: | 2581 | IREKAGHWFATSTPIIGKGIMFAVKKGRVTTGISSIATDDSRKIASVLNSAHYLEKMHYS | 2640 |
| QUERY: | 2572 | IEGKDTHYFVKIGSADGDLVTLGTTIGRKVLESGVNVTVSQPTLLVNGRTRRFTNIEFQY | 2631 |
| | | \|\|\|\|\|\|\|\|\|\|\|\|\|\|\|\| \|\|\|\|\| \| \|\|\|\|\|+\|\|\|\|\|\|\|\|\|\|\|\|\|+\|\|\|\|\|\|\|\|\|\|\|\|\|\| | |
| SBJCT: | 2641 | IEGKDTHYFVKIGSADSDLVTLAMTSGRKVLDSGVNVTVSQPTLLINGRTRRFTNIEFQY | 2700 |
| QUERY: | 2632 | STLLLSIRYGLTPDTLDEEKARVLDQARQRALGTAWAKEQQKARDGREGSRLWTEGEKQQ | 2691 |
| | | \|\|\|\|++\|\|\|\|\|\| \|\|\|\|\|\|\|\|\|\|\|\|\|\|\|\|\|\|\|\|+\|\|\|\|\|\|\|\|\|\|\|\|\|\|\|\|\|+\|\|+\|\|\|\|\| | |
| SBJCT: | 2701 | STLLINIRYGLTADTLDEEKARVLDQARQRLGSAWAKEQQKARGREGSRVWTDGEKQQ | 2760 |
| QUERY: | 2692 | LLSTGRVQGYEGYYVLPVEQYPELADSSSNIQFLRQNEMGKR 2733 | |
| | | \|\|+\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 2761 | LLNTGRVQGYEGYYVLPVEQYPELADSSSNIQFLRQNEMGKR 2802 | |

The FCTR3bcde and f amino acid sequences have 1524 of 2352 amino acid residues (64%) identical to, and 1881 of 2532 residues (79%) positive with, the amino acid residues 429-2771, 93 of 157 residues (59%) identical to and 118 of 157 residues (74%) positive with amino acid residues 1-155, and 59 of 152 residues (38%) identical to and 68 of 152 residues (43%/O) positive with amino acid residues 211-361 of Ten-m4 [Mus musculus] (ptnr: GenBank Acc: BAA77399.1) (SEQ ID NO:70) (Table 3R).

TABLE 3R
BLASTP of FCTR3b, c, d, e, and f against Mus musculus Ten-m4 - (SEQ ID NO:70)

>GI|4760782|DBJ|BAA77399.1| (AB025413) TEN-M4 [MUS MUSCULUS]
LENGTH = 2771
SCORE = 3089 BITS (8008), EXPECT = 0.0
IDENTITIES = 1524/2352 (64%), POSITIVES = 1881/2352 (79%), GAPS = 28/2352 (1%)

| | | | |
|---|---|---|---|
| QUERY: | 401 | KNSSIDSGEAEVGRRVTQEVPPGVFWRSQIHISQPQFLKFNISLGKDALFGVYIRRGLPP | 460 |
| | | ++\| \|\|\|\|\| +\|\|\|\| +\|++\|\|\| \|\|\|\|\|+ \| \| \|\|\|\|+\|\|\|\| \|\| \|+\| \|+\|\|\|\| | |
| SBJCT: | 429 | EDSFIDSGEIDVGRRASQKIPPGTFWRSQVFIDHPVHLKFNVSLGKAALVGIYGRKGLPP | 488 |
| QUERY: | 461 | SHAQYDFMERLDGK-----EKWSVESPR3RRSIQTLVQNEAVFVQYLDVGLWHLAFYND | 515 |
| | | \|\| \|+\|\|+\| \|\|\|+ \| \|+ \|+ \| +\| \|+\|\|\|\| \|+\|\|\|\|\|\|\|\| | |
| SBJCT: | 489 | SHTQFDFVELLDGRRLLTQEARSLEGPQRQSRGPVPPSSHETGFIQYLDSGIWHLAFYND | 548 |
| QUERY: | 516 | GKDKEMVSFNTLDSVQDCPENCHGNGECVSGVCECFPGFLGADCAKAACPVLCSGNGQ | 575 |
| | | \|\|+ \|+\|\|\| \| ++\|\| +\|\| \|\|+\|\|\|+\|+\|\| \|\|\|\| \|\|\|\| \|\| +\|+\|\|\|\|\|\|\|\|\|\| | |
| SBJCT: | 549 | GKESEVVSFLTTAIESVDNCPSNCYGNGDCISGTCHCFLGFLGPDCGRASCPVLCSGNGQ | 608 |
| QUERY: | 576 | YSKGTCQCYSGWKGAECDVPMMQCIDPSCGGHGSCIDGNCVCSAGYKGEHCEEVDCLDPT | 635 |
| | | \| \|\| \| \|+\|\|\|\|\|\|\|\|\|\|\| \|\|\|\|\| +\| \|\|+\|\| \| \|+\|+ \|\|\|\|\| \|\|\|\|\|\|+\|\|\| | |
| SBJCT: | 609 | YMKGRCLCESGWKGAECDVPTNQCIDVACSSEGTCIMGTCICNPGYKGESCEEVDCMDPT | 668 |
| QUERY: | 636 | CSSHGVCVNGECLCSPGWGGLNCELARVQCPDQCSGEGTYLPDTGLCSCDPNWMGPDCSV | 695 |
| | | \|\|\| \|\|\|\| \|\|\| \|\| \|\|\|\| \|\|\| \| \| \|\|\|\|\|\|\|\|+\|\|\|\|\|\|\|+\|\|\|+\| \| \|\|\|+ | |
| SBJCT: | 669 | CSSRGVCVRGECHCSVGWGGTNCETPRATCLDQCSGHGTFLPDTGLCNCDPSWTGHDCSI | 728 |
| QUERY: | 696 | EVCSVDCGTHGVCIGGACRCEEGWTGAACDQRVCHPRCIEHGTCKDGKCECREGWNGEHC | 755 |
| | | \|+\|+ \|\|\| \|\|\|\|+\|\| \|\|\|\|+\|\| \|\|\|\|\|\|\| \|\|\|\|\| \|\|\|\|\|+\|\|\|\|\|\| \|\|\|\|\|\|\| | |
| SBJCT: | 729 | EICAADCGGHGVCVGGTCRCEDGWMGAACDQRACHPRCAEHGTCRDGKCECSPGWNGEHC | 788 |
| | | ********* | |
| QUERY: | 756 | TIGRQTAGTETDGCPDLCNGNGRCTLGQNSWQCVCQTGWRGPGCNVAMETSCADNKDNEG | 815 |
| | | \|\| +\|\|\| \|\|\|\|\|\|\|\|\|\| \| \| \|\|\|\| \|\|+ +\|\|\| \| \| \|\|\|+\| | |
| SBJCT: | 789 | TIAHYLDRVVKEGCPGLCNGNGRCTLDLNGWHCVCQLGWRGTGCDTSMETGCGDGKDNDG | 848 |
| QUERY: | 816 | DGLVDCLDPDCCLQSACQNSLLCRGSRDPLDIIQQGQT--DWPAVKSFYDRIXLLAGKDS | 873 |
| | | \|\|\|\|\|\|+\|\|\|\|\|\| \| + \|\| \|\| \|\|\|\|\|\|\|+ \| + \|\|\|\|\|\| \| \|+\|\| | |
| SBJCT: | 849 | DGLVDCMDPDCCLQPLCHVNPLCLGSPDPLDIIQETQAPVSQQNLNPFYDRIKFLVGRDS | 908 |
| QUERY: | 874 | THIIPGENPFNSSLVSLIRGQVVTTDGTPLVGVNVSFVKYPKYGYTITRQDGTFDLIANG | 933 |
| | | \|\| \|\|\|\|\|\|\|+ +\|\|\|\|\|+\|+\|\|\|\|\|\|\|\|\|+\|\|+ \| +\|\|\|\|+\|\|\|\|+\|\|\|+ \|\| | |
| SBJCT: | 909 | THSIPGENPFDGGHACVIRGQVMTSDGTPLVGVNISFINNPLFGYTISRQDGSFDLVTNG | 968 |
| QUERY: | 934 | GASLTLHFERAPFMSQERTVWLPNNSFYAMDTLVMKTEENSIPSCDLSGFVRPDPIIISS | 993 |
| | | \| \|+ \| \|\|\|\|\|\|++\|\| \|+\|\|\|\|+ \|+ \|+\|+\|\|+ \|\|\| \|\|\|\|\|\|\| \| \|\|+\|++ \| | |
| SBJCT: | 969 | GISIILRFERAPFITQEHTLWLPWDRFFVMETIVMRHEENEIPSCDLSNFARPNPVVSPS | 1028 |
| QUERY: | 994 | PLSTFFSAAPGQNPIVPETQVLHEEIELPGSNVKLRYLSSRTAGYKSLLKITMTQSTVPL | 1053 |
| | | \|\|++\| \|+ + \|\|\|\|\| \| \| \|\|\| + \| ++\| \|\|\|\|\|\| \|\|\|\|+\|+\|++\| \|+\| | |
| SBJCT: | 1029 | PLTSFASSCAEKGPIVPEIQALQEEIVIAGCKMRLSYLSSRTPGYKSVLRISLTHPTIPF | 1088 |
| QUERY: | 1054 | NLIRVHLMVAVEGHLFQKSFQASPNLASTFIWDKTDAYGQRVYGLSDAVVSVGFEYETCP | 1113 |
| | | \|\|++\|\|\|\|\|\|\|\|\| \|\|+\| \| \|+\|+\|+ \|\|\|\|\|\|\| \| \|+\|+\| \|+\| \|\|\|\|+\|\|\|+\|\| | |
| SBJCT: | 1089 | NLMKVHLMVAVEGRLFRKWFAAAPDLSYYFIWDKTDVYNQKVFGFSEAFVSVGYEYESCP | 1148 |
| QUERY: | 1114 | SLILWEKRTALLQGFELDPSNLGGWSLDKHHILNVKSGILHKGTGENQFLTQQPAIITSI | 1173 |
| | | \|\|\|\|\|\|\|\|\|+\|\|\|+\|+\| \| \|\|\|\|\|\|\|\|\|\| \|\|++\|\|\|\|\|\|\| \|\|\|\|\|++\|\|\| +\| \|\| | |
| SBJCT: | 1149 | DLILWEKRTAVLQGYEIDASKLGGWSLDKHHALNIQSGILHKGNGENQFVSQQPPVIGSI | 1208 |
| QUERY: | 1174 | MGNGRRRSISCPSCNGLAEGNKLLAPVALAVGIDGSLYVGDFNYIRRIFPSRNVTSILEL | 1233 |
| | | \|\|\|\|\|\|\|\|\|\|\|\|\|\|\|\|\|\|+\|\|\|\|\|\|\|\|\|\| \| \|\|\|\|\|\|\|\|\|\|\|\|\|\|\|\|\|\| \|\|\|+\|\|\|+ | |
| SBJCT: | 1209 | MGNGRRRSISCPSCNGLADGNKLLAPVALTCGSDGSLYVGDFNYIRRIFPSGNVTNILEM | 1268 |
| QUERY: | 1234 | RNKEWKHSNNPAHKYYLAVDPVSGSLYVSDTNSRRIYRVKSLSGTKDLAGNSEVVAGTGE | 1293 |
| | | \|\|\|+\|+\|\|++\|\|\|\|\|\|\|\| \|\|+\|\|++++\|\|\|\|\|\|\|+++\|\|\| + \|\|\| \|\|\|\|\|\|\|\|\|+ | |
| SBJCT: | 1269 | RNKDFRBSHSPABKYYLATDPMSGAVFLSDTNSRRVFKVKSTTVUEDLVKNSEVVAGTGD | 1328 |
| QUERY: | 1294 | QCLPFDEARCGDGGKAIDATLMSPRGIAVDKNGLMYFVDATMIRKVDQNGIISTLLGSND | 1353 |
| | | \|\|\|\|\|\|+ \|\|\|\|\|\|\|\| +\|\|\| +\|\|\|\| \|\|\| \|\|+\|\|\|\| \|\|\|\|+\|\|\|\|\|\|\|\|\|\|\|\|\|\|\| | |
| SBJCT: | 1329 | QCLPFDDTRCGDGGATEATLTNPRGITVDKFGLIYFVDGTMIRRVDQNGIISTLLGSND | 1388 |
| QUERY: | 1354 | LTAVRPLSCDSSMDVAQVRLEWPTDLAVNPMDNSLYVLENNVILRITENEQVSIIAGRPM | 1413 |
| | | \|\|+ \|\|\|\|\|\|\| \|+++\|\|\|\|\|\|\|\|\|\|\|+\|\|\|\|\|\|\|\|\|\|+\|\|\|+\|+\|+\|\|\|\|\| \|+\|\|\|\|\| | |
| SBJCT: | 1389 | LTSARPLSCDSVMEISQVRLEWPTDLAINPMDNSLYVLDNNVVLQISENHQVRIVAGRPM | 1448 |
| QUERY: | 1414 | HCQVPGID-YSLSKXXXXXXXXXXXXXXXXXXGVLYITETDEKKINRLRQVTTGEICLL | 1472 |
| | | \|\|\|\|\|\|\|\| + \|\|\| \|\|\|\|\| \|\|\|\|\|\|\|\|\|+\|\|\|\|\|+\|\|\| \|+ | |
| SBJCT: | 1449 | HCQVPGIDHFLLSKVAIHATLESATALAVSHNGVLYIAETDEKKINRIRQVTTSGEISLV | 1508 |
| QUERY: | 1473 | AGAASXXXXXXXXXXXXXYSGDDAYATDAILNSPSSLAVAPDGTIYIADLGNIRIRAVSKN | 1532 |
| | | \|\|\| \| +\|\|\|\| \|\| \|\| \|\|+\|\|\|\|\| \|\| +\|+\|\|\|\|\|\|\|\|\| + \|\| | |
| SBJCT: | 1509 | AGAPSGCDCKNDANCDCFSGDDGYAKDAKLNTPSSLAVCADGELYVADLGNIRIRFIRKN | 1568 |
| QUERY: | 1533 | KPVLNAFNQYEAASPGEQELYVFNADGIHQYTVSLVTGEYLYNFTYSTDNDVTELIDNNG | 1592 |
| | | \|\| \|\| \| \|\| +\|\| +\|\|\|\|+\|+ \| \| \|\| \|\| \|\|+\|\|\|\|\|\|\|+ \| \|+\| + \|\|\|\| | |
| SBJCT: | 1569 | KPFLNTQNMYELSSPIDQELYLFDTSGKHLYTQSLPTGDYLYNFTYTGDGDITEITDNNG | 1628 |
| QUERY: | 1593 | NSLKIRRDSSGMPRHLLMPDNQIITLTVGTNGGLKVVSTQNLELGLMTYDGNTGLLATKS | 1652 |
| | | \| + +\|\|\|\|+\|\|\| \|++\|\| \|+ +\|+\|\|\| \|+ \|+\|\| \|\| +\|\|\| \|\|+\|\|\|\|\|\|\| | |
| SBJCT: | 1629 | NMVNVRRDSTGMPLWLVVPDGQVYWVTMGTNSALRSVTTQGHELAMMTYHGNSGLLATKS | 1688 |
| QUERY: | 1653 | DETGWTTFYDYDHEGRLTNVTRPTGVVTSLEREMEICSITIDIENSNEDDDVTVITNLSSV | 1712 |
| | | +\| \|\|\|\|\|\|+\|\| \|\|\|\|\|\|\| \|\|\| \|+\| + + \|+ + +\| \|++ \|\|\|\|+ \|\|\|\|+ | |
| SBJCT: | 1689 | NENGWTTFYEYDSFGRLTNVTFPTGQVSSFRSDTDSSVHVQVETSSK-DDVTITTNLSAS | 1747 |
| QUERY: | 1713 | EASYTVVQDQVRNSYQLCNNGTLRVMYANGMGISFHSEPHVLAGTITPTIGRCNISLPME | 1772 |
| | | \| \|\|++\|\|\|\|\|\|\|\| + +\|+\|\|++ \|\|\|\| ++ +\|\|\|+\|\|\|\|+ \|\|+\|+ \|++\|\|++ | |
| SBJCT: | 1748 | GAFYTLLQDQVRNSYYIGADGSLRLLLANGNEVALQTEPHLLAGTVNPTVGKRNVTLPID | 1807 |
| QUERY: | 1773 | NGLNSIEWRLRKEQIKGKVTIFGRKLRVHGRNLLSIDYDRNIRTEKIYDDHRKFTLRIIY | 1832 |
| | | \|\|\|\| +\|\|\| \|\|\|\| +\|+\|\|+\|\|\|+\|\|\|\| \|\|\|\|\|+\|+\|\| \|\|\|\|\|\|\|\|\|\|\|\|\|\|\|\|+\| | |
| SBJCT: | 1808 | NGLNLVEWRQRKEQARGQVTVFGRRLRVENRNLLSLDFDRVTRTERIYDDHRRFTLRILY | 1867 |
| QUERY: | 1833 | DQVGRPFLWLPSSGLAAVNVSYFFNGRLAGLQRGAI4SERTDIDKQGRIVSRMFADGKVWS | 1892 |
| | | \|\| \|\|\| \|\| \|\|\| \| \|\|\|+\| \| +\|\|+\|\|\| \|\|\|\| + \|+ \|\|\| \|\|+\|\|\|\|\|+\|\| | |
| SBJCT: | 1868 | DQAGRPSLWSPSSRLNGVNVTYSPGGHIAGIQRGIMSERMEYDQAGRITSRIFADGKMWS | 1927 |
| QUERY: | 1893 | YSYLDESMVLLLQSQRQYIFEYDSSDRLLAVTMPSVARESMSTHTSIGYIRNIYNPPESN | 1952 |
| | | \|+\|\|+\|\|\|\|\| \| \|\|\|\|\|\|\|\|+\| +\|\|\| +\|\|\|\|+\|\|\| ++ \| \|+\|\| \|\|\|\| \|\|\| \| | |
| SBJCT: | 1928 | YTYLEKSMVLHLHSQRQYIFEFDKNDRLSSVTMPNVARQTLETIRSVGYYRNIYQPPEGN | 1987 |
| QUERY: | 1953 | ASVIFDYSDDGRILKTSFLGTGRQVFYKYGKLSKLSEIVYDSTAVTFGYDETTGVLKNVN | 2012 |
| | | \|\|\|\| \|+++\|\| +\| \| +\|\|\|\|\|+\| \|\|\|\|\|\|\|\|\|+\| +\|\|+\| \|\|\|\| \|+\|\| \|\| | |
| SBJCT: | 1988 | ASVIQDFTEDGHLLHTFYLGTGRRVIYKYGKLSKLAETLYDTTKVSFTYDETAGMLKTVN | 2047 |
| QUERY: | 2013 | LQSGGFSCTIRYRKIGPLVDKQIYRFSEEGMVNARFDYTYRDNSFRIASIKPVISETPLP | 2072 |
| | | \|\|+ \|\|+\|\|\|\|\|\|+\|\|\|\|+\|+\|\|+\|\|+\|\|\|\|\|\|\|\|\|\|\| \| \|\|\|\|\|+ \|++ \|\|+\|\|\|\|\| | |
| SBJCT: | 2048 | LQNEGFTCTIRYRQIGPLIDRQIFRFTEEGMVNARFDYNYDNSFRVTSMQAVINETPLP | 2106 |
| QUERY: | 2073 | VDLYRYDEISGKVEHFGKFGVIYYDINQIITTAVMTLSKHFDTHGRIKEVQYEMFRSLMY | 2132 |
| | | +\|\|\|\|\|\|++\|\|\| \| \|\|\|\|\|\|\|\|\|\|\|\|\|\|\|\|\|\|\|\|\| +\|\|\|\| +\|\|+\|\|\|\|\|\|+\|\|\|\|\|\| | |
| SBJCT: | 2107 | IDLYRYDDVSGKTEQFGKfGVIYYDINQIITTAVMTHTKHFDAYGRMKEVQYEIFRSLMY | 2166 |
| QUERY: | 2133 | WMTVQYDSMGRVIKRELKLGPYANTTKYTYDYDGDGQLQSVAVNDRPTWRYSYDXXXXXX | 2192 |
| | | \|\|\|\|\|\|\|+\|\|\|\|+\|+\|\|\|+\|\|\|\|\|\|\|+\|+\|+\|\| \|\|\|\|\|+\|++\|\|+\| \|\|\|\|\|\| | |
| SBJCT: | 2167 | WMTVQYDNMGRVVKKELKVGPYANTTRYSYEYDADGQLQTVSINDKPLWRYSYDLNGNLH | 2226 |
| QUERY: | 2193 | XXXXXXSVRLMPLRYDLRDRITRLGDVQYKIDDDGYLCQRGSDIFEYNSKGLLTRAYNKA | 2252 |
| | | \| \|\| \|\|\|\|\|\|\|\|\|\|\|\|\|\|\|\|\|\|\|+\|+\|\|+\| \|\|\| \|+\|\|\|\|\| \|\|\| +\|\|\|+\| | |
| SBJCT: | 2227 | LLSPGNSARLTPLRYDLRDRITRLGDVQYKMDEDGFLRQRGGDVFEYNSAGLLIKAYNRA | 2286 |
| QUERY: | 2253 | SGWSVQYRYDGVGRRASYKTNLGHHLQYFYSDLHMPTRITHVYNHSNSEITSLYYDLQGE | 2312 |
| | | \|\|\|\|\|+\|\|\|\|\|+\|\|\| \| \|++ \|\|\|\|+\|\|+\|\| \|\|\|++\|\|+\|\|\|\|+\|\|\|\|\|\|\|\|\|\|\|\|\| | |
| SBJCT: | 2287 | SGWSVRYRYDGLCRRVSSKSSESHHLQFFYADLTNPTKVTELYNHSSSEITSLYYDLQGE | 2346 |
| QUERY: | 2313 | LFAMESSSGEEYYVASDNTGTPLAVFSINGLMIKQLQYTAYGEIYYDSNPDFQMVIGFEG | 2372 |
| | | \|\|\|\|\| \|\|\|+\|+\|+\| \|\| \|\|\|\|\|\|\|\| \|\|\|\|\|\|+ \|\|\|\|\|\|\|\| \|+\|\|+\|\|++\|\|+\|\| | |
| SBJCT: | 2347 | LFAMELSSGDEFYIACDNIGTPLAVFSGTGLMIKQILYTAYGEIYMDTNPNFQIIIGYEG | 2406 |
| QUERY: | 2373 | GLYDPLTKLVHFTQRDYDVLAGRWTSPDYTMWKNVGKEP-APFNLYMFKSNNPLSSELDL | 2431 |
| | | \|\|\|\|\|\|\|\|\|\|\| +\|\|\|\|\|\|\|\|\|\|\|\|\|\|+ +\|\| + \|\|+\|\|\|\|\|+\|\|\|+\|+ \|+ | |
| SBJCT: | 2407 | GLYDPLTKLVENGRRDYDVLAGRWTSPDHELWKRLSSNSIVPFHLYMFKNNNPISNSQDI | 2466 |
| QUERY: | 2432 | KNYVTDVKSWLVMFGFQLSNIIPGFPRARMYFVPPPYELSESQAS----ENGQLITGVQQ | 2487 |
| | | \| ++\|\|\| \|\|\|+ \|\|\|\|\| \|+\|\|\|+\|+ + \| \|\|\| +\| +\| + \| \|\|\| | |
| SBJCT: | 2467 | KCFMTDVNSWLLTFGFQLHNVIPGYPKPDTDAMEPSYELVETQMKTQEWDNSKSILGVQC | 2526 |
| QUERY: | 2488 | TTERHNQAFMALE------GQVITKKLHASIREKAGEWFATTTPIIGKGIMFAIKEGRVT | 2541 |
| | | ++ +\|\|+ \|\| \| \|\| \| +\| \|\|++ \| \|\|\|+ \|\|+\|+\|\|\|\| | |
| SBJCT: | 2527 | EVQKQLKAFVTLERFDQLYGSTITSCQQAPETKK----FASSGSIFGKGVKFALKDGRVT | 2582 |
| QUERY: | 2542 | TGVSSIASEDSRKVASVLNNAYYLDKMHYSIEGKDTHYFVKIGSADGDLVTLGTTIGRKV | 2601 |
| | | \| + \|+\|+\|\| \|++\|++\|\|\|\|+\|\|+ +\|++\|+\| \|\|\|\|\|\|\| \| ++\|\|\| \|\| + \|\|+ | |
| SBJCT: | 2583 | TDIISVANEDGRRIAAILNNAHYLENLHFTIDGVDTEYFVKPGPSEGDLAILGLSGGRRT | 2642 |
| QUERY: | 2602 | LESGVNVTVSQPTLLVNGRTRRFTNIEFQYSTLLLSIRYGLTPDTLDEEKARVLDQARQR | 2661 |
| | | \|\|+\|\|\|\|\|\|\|\| +++\|\|\|\|\|+\|+\|+ \|\| \| \|+ \|\|\| \|+\|\|\|\| \|\|\|+ \|\|\|\| | |
| SBJCT: | 2643 | LENGVNVTVSQINTNLSGRTRRYTDIQLQYRALCLNTRYG---TTVDEEKVRVLELARQR | 2699 |
| QUERY: | 2662 | ALGTAWAKEQQRARDGREGSRLWTEGEKQQLLSTGRVQGYEOYYVLPVEQYPELADSSSN | 2721 |
| | | + \|\|\|+\|\|\|+ \|+\| \|\| \| \|\|+\|\|\|\|\|+\|+\|\|\|\|\|\|\|+\|++\| \|\|\|\|\|\|\|+\|\|++\| | |
| SBJCT: | 2700 | AVRQAWAREQQRLREGEEGLRAWTDGEKQQVLNTGRVQGYDGFFVTSVEQYPELSDSANN | 2759 |
| QUERY: | 2722 | IQFLRQNEMGKR 2733 | |
| | | \| \|+\|\|+\|\|\|+\| | |
| SBJCT: | 2760 | IHFMRQSEMGRR 2771 | |

SCORE = 161 BITS (407), EXPECT = 2E − 37
IDENTITIES = 93/157 (59%), POSITIVES = 118/157 (74%), GAPS = 4/157 (2%)

| | | | |
|---|---|---|---|
| QUERY: | 1 | MDVKDRR-HRSLTRGRCGKECRYTSSSLDSEDCRVPTQKSYSSSETLKAYDEDSRMEYGN | 59 |
| | | \|\|\|\|+\|+ +\|\|\|\|\| \| \| \|\|\|\|\|\| \|\|\|+ + \| \|\|\|\|\|\|\|\|\|\|\|\|\|\| \|+\|+ \|\|+ | |
| SBJCT: | 1 | MDVKERKPYRSLTRRR-DAERRYTSSSADSEEGKGP-QKSYSSSETLKAYDQDARLAYGS | 58 |
| QUERY: | 60 | RVTDLIHRESDEFPRQGTNFTLELGICEPS-PHRSGYCSDNGILHQGYSLSTGSDADSD | 118 |
| | | \|\| \|++ +\|++\|\| \| \|\|\|\|\|\| \|\|\|+ \| + \|\| +\| +\|+\|+\|+ \| \|\|\|+ \|\|\|\| + | |
| SBJCT: | 59 | RVKDMVPQEAEEFCRTGTNFTLRELGLGEMTPPHGTLYRTDIGLPHCGYSNGASSDADLE | 118 |
| QUERY: | 119 | TEGGMSPEHAIRLWGRGIKSRRSSGLSSRENSALTLT 155 | |
| | | + +\|\|\|\| +\|\|\|\|\| +\| \|\|\| \|\|\|\| \|\| \|\|\|\| | |
| SBJCT: | 119 | ADTVLSPEHPVRLWGRSTRSGRSSCLSSRANSNLTLT 155 | |

SCORE = 72.1 BITS (176), EXPECT = 8E − 11
IDENTITIES = 59/152 (38%), POSITIVES = 68/152 (43%), GAPS = 42/152 (27%)

| | | | |
|---|---|---|---|
| QUERY: | 285 | PAPAPND--LATTP------ESVQLQDSWVLNSNVPLETR-------------------- | 316 |
| | | \|+\|\|\| \| \|+ \| \| \|\|+\|+\|\|\|\|+\|\|\|\|\| | |
| SBJCT: | 211 | PSPAPTDESLSGEPPAGSAQEPTEAQDNWLLNSNIPLEVENLGEQPFLGTLQDNLIEMDI | 270 |
| QUERY: | 317 | -------------HFLFKXXXXXXXXXXXXXXXYPLTSGTVYTPPPRLLPRNTFSRKAFK | 363 |
| | | \|\|\|\|\| \|\|\|\|\| \|\|\|+\|\|\|\| \|\|\|+\|\|\|\| \|\| | |
| SBJCT: | 271 | LSASRHDGAYSDGHFLFKPGGTSPLFCTTSPGYPLTSSTVYSPPPRPLPRSTFSRPAFN | 329 |
| QUERY: | 364 | LKKPSKYCYCSWKCXXXXXXXXXXXXXXXXXYFI 395 | |
| | | \|\|\|\|\|\|\|\|+\|\|\| \|\|+ | |
| SBJCT: | 330 | LKKPSKYCNWKCAALSAILISATLVILLAYFV 361 | |

*FCTR3F DOES NOT CONTAIN THESE AMINO ACIDS

The 997-2733 amino acid fragment of the FCTR3bcde and f protein was also found to have 1695 of 1737 amino acid residues (97%) identical to, and 1695 of 1737 residues (97%) positive with the amino a 1737 amino acid residue protein KIAA1 127 protein [Homo sapiens] (GenBank Acc:(AB032953) (SEQ ID NO:71), (Table 3S).

TABLE 3S

BLASTP of FCTR3b, c, d, e, and f against Homo sapiens KIAA1127 protein
(SEQ ID NO:71)

>GI|6329763|DBJ|BAA86441.1| (AB032953) K1AA1127 PROTEIN [HOMO SAPIENS]
LENGTH = 1737
SCORE = 3295 BITS (8545), EXPECT = 0.0
IDENTITIES = 1695/1737 (97%), POSITIVES = 1695/1737 (97%)

| | | | |
|---|---|---|---|
| QUERY: | 997 | TFFSAAPGQNPIVPETQVLHEEIELPGSNVKLRYLSSRTAGYKSLLKITMTQSTVPLNLI | 1056 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 1 | TFFSAAPGQNPIVPETQVLHEEIELPGSNVRLRYLSSRTAGYKSLLKITMTQSTVPLNLI | 60 |
| QUERY: | 1057 | RVHLMVAVEGHLFQKSFQASPNLASTFIWDKTDAYGQRVYGLSDAVVSVGFEYETCPSLI | 1116 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 61 | RVHLMVAVEGHLFQKSFQASPNLAYTFIWDKTDAYGQRVYGLSDAVVSVGFEYETCPSLI | 120 |
| QUERY: | 1117 | LWEKRTALLQGFELDPSNLGGWSLDKHHILNVKSGILHKGTGENQFLTQQPAIITSIMGN | 1176 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 121 | LWEKRTALLQGFELDPSNLGGWSLDKHHILNVKSGILEKGTGENQFLTQQPAIITSIMGN | 180 |
| QUERY: | 1177 | GRRRSISCPSCNGLAEGNKLLAPVALAVGIDGSLYVGDFNYIRRIFPSRNVTSILELRNK | 1236 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 181 | GRRRSISCPSCNGLAEGNKLLAPVALAVGIDGSLYVGDFYIRRIFPSRNVTSILELRNK | 240 |
| QUERY: | 1237 | EFKHSNNPAHKYYLAVDPVSGSLYVSDTNSRRIYRVKSLSGTKDLAGNSEVVAGTGEQCL | 1296 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 241 | EFKHSNNPAHKYYLAVDPVSGSLYVSDTNSRRIYRVKSLSGTKDLAGNSEVVAGTGEQCL | 300 |
| QUERY: | 1297 | PFDEARCGDGGKAIDATLMSPRGIAVDKNCLMYFVDATMIRKVDQNGIISTLLGSNDLTA | 1356 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 301 | PFDEARCGDGGICAIDATLMSPRGIAVDKNGLMYFVDATMIRKVDQNGIISTLLGSNDLTA | 360 |
| QUERY: | 1357 | VRPLSCDSSMDVAQVRLEWPTDLAVNPMDNSLYVLENNVILRITENHQVSIIAGRPMHCQ | 1416 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 361 | VRPLSCDSSMDVAQVRLEWPTDLAVNPMDNSLYVLENNVILRITENHQVSIIAGRPMHCQ | 420 |
| QUERY: | 1417 | VPGIDYSLSKXXXXXXXXXXXXXXXXXTGVLYITETDEKKINRLRQVTTNGEICLLAGAA | 1476 |
| | | \|\|\|\|\|\|\|\|\|\| \|\|\|\|\|\|\|\|\|\|\|\|\|\|\|\|\|\|\|\|\|\|\|\|\|\|\|\|\|\|\|\|\| | |
| SBJCT: | 421 | VPGIDYSLSKLAIHSALESASAIAISHTGVLYITETDEKKINRLRQVTTNGEICLLAGAA | 480 |
| QUERY: | 1477 | SXXXXXXXXXXXXYSGDDAYATDAILNSPSSLAVAPDGTIYIADLGNIRIRAVSKNKPVL | 1536 |
| | | \| \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 481 | SDCDCKNDVNCNCYSGDDAYATDAILNSPSSLAVAPDGTIYIADLGNIRIRAVSKNKPVL | 540 |
| QUERY: | 1537 | NAFNQYEAASPGEQELYVFNADGIRQYTVSLVTGEYLYNFTYSTDNDVTELIDNNGNSLK | 1596 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 541 | NAFNQYEAASPGEQELYVFNADGIHQYTVSLVTGEYLYNFTYSTDNDVTELIDNNGNSLK | 600 |
| QUERY: | 1597 | IRRDSSGMPRELLMPDNQIITLTVGTNGGLKVVSTQNLELGLMTYDGNTGLLATKSDETG | 1656 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 601 | IRRDSSGMPRHLLMPDNQIITLTVGTNGGLKVVSTQNLELGLMTYDGNTGLLATKSDETG | 660 |
| QUERY: | 1657 | WTTFYDYDHEGRLTNVTRPTGVVTSLHREMEKSITIDIENSNRDDDVTVITNLSSVEASY | 1716 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 661 | WTTFYDYDHEGRLTNVTRPTCVVTSLHREMEKSITIDIENSNRDDDVTVITNLSSVEASY | 720 |
| QUERY: | 1717 | TVVQDQVRNSYQLCNNGTLRVMYANGMGISFHSEPHVLAGTITPTIGRCNISLPMENGLN | 1776 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 721 | TVVQDQVRNSYQLCNNGTLRVMYANGMGISFHSEPRVLAGTITPTIGRCNISLPNENGLN | 780 |
| QUERY: | 1777 | SIEWRLRKEQIKGKVTIFGRKLRVHGRNLLSIDYDRNIRTEKIYDDHRKFTLRIIYDQVG | 1836 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 781 | SIEWRLRKEQIKGKVTIFGRKLRVHGRNLLSIDYDRNIRTEKIYDDHRKFTLRIIYDQVG | 840 |
| QUERY: | 1837 | RPFLWLFSSGLAAVNVSYFFNGRLAGLQRGAIASERTDIDKQGRIVSRMFADGKVWSYSYL | 1896 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 841 | RPFLWLPSSGLAAVNVSYFFNGRLAGLQRGAMSERTDIDKQGRIVSRMFADGKVWSYSYL | 900 |
| QUERY: | 1897 | DKSMVLLLQSQRQYIFEYDSSDRLLAVTMPSVARHSMSTHTSIGYIRNIYNPPESNASVI | 1956 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 901 | DKSMVLLLQSQRQYIFEYDSSDRLLAVTMPSVARHSMSTHTSIGYIRNIYNPPESNASVI | 960 |
| QUERY: | 1957 | FDYSDDGRILKTSFLGTGRQVFYKYGKLSKLSEIVYDSTAVTFGYDETTGVLKMVNLQSG | 2016 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 961 | FDYSDDGRILKTSFLGTGRQVFYKYGKLSKLSEIVYDSTAVTFGYDETTGVLKMVNLQSG | 1020 |
| QUERY: | 2017 | GFSCTIRYRKIGPLVDKQIYRFSEEGMVNARFDYTYHDNSFRIASIKPVISETPLPVDLY | 2076 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 1021 | GFSCTIRYRKIGPLVDKQIYRFSEEGMVNARFDYTYRDNSFRIASIKPVISETPLPVDLY | 1080 |
| QUERY: | 2077 | RYDEISGKVEHFGKFGVIYYDINQIITTAVMTLSKHFDTHGRIKEVQYEMFRSLMYWNTV | 2136 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 1081 | RYDEISGKVEHFGKFGVIYYDINQIITTAVMTLSKHFDTRGRIKEVQYEMFRSLMYWMTV | 1140 |
| QUERY: | 2137 | QYDSNGRVIKRELKLGPYANTTKYTYDYDGDGQLQSVAVNDRPTWRYSYDXXXXXXXXXX | 2196 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 1141 | QYDSNGRVIKRELKLGPYANTTKYTYDYDGDGQLQSVAVNDRPTWRYSYDLNGNLHLLNP | 1200 |
| QUERY: | 2197 | XXSVRLMPLRYDLRDRITRLGDVQYKIDDDGYLCQRGSDIFEYNSKGLLTRAYNKASGWS | 2256 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 1201 | GNSVRLMPLRYDLRDRITRLGDVQYKIDDDGYLCQRGSDIFEYNSKGLLTRAYNKASGWS | 1260 |
| QUERY: | 2257 | VQYRYDGVGRRASYKTNLGRHLQYFYSDLHNPTRITRVYNHSNSEITSLYYDLQGRLFAM | 2316 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 1261 | VQYRYDGVGRRASYKTNLGHRLQYFYSDLHNPTRITHVYNHSNSEITSLYYDLQGHLFAM | 1320 |
| QUERY: | 2317 | ESSSGEEYYVASDNTGTPLAVFSINGLMIRQLQYTAYGEIYYDSNPDFQMVIGFHGGLYD | 2376 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 1321 | ESSSGEEYYVASDNGTPLAVFSINGLMIKQLQYTAYGEIYYDSNPDFQMVIGFHGGLYD | 1380 |
| QUERY: | 2377 | PLTKLVRFTQRDYDVLAGRWTSPDYTMWKNVGKEPAPFNLYMFKSNNPLSSELDLKNYVT | 2436 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 1381 | PLTKLVHFTQRDYDVLAGRWTSPDYTMWKNVGKEPAPFNLYMFKSNNPLSSELDLKNYVT | 1440 |
| QUERY: | 2437 | DVKSWLVMFGFQLSNIIPGFPRAKMYFVPPPYELSESQASENGQLITGVQQTTERHNQAF | 2496 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 1441 | DVXSWLVMFGFQLSNIIPGFPRAKMYFVPPPYELSESQASENGQLITGVQQTTERHNQAF | 1500 |
| QUERY: | 2497 | MALEGQVITKKLHASIREKAGHWFATTTPIIGRGIMFAIKEGRVTTGVSSIASEDSRKVA | 2556 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 1501 | NALEGQVITKKLHASIREKAGRWFATTTPIIGKGIMFAIKEGRVTTGVSSIASEDSRKVA | 1560 |
| QUERY: | 2557 | SVLNNAYYLDKMHYSIEGKDTHYFVKIGSADGDLVTLGTTIGRKVLESGVNVTVSQPTLL | 2616 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 1561 | SVLNNAYYLDKMHYSIEGKDTHYFVKIGSADGDLVTLGTTIGRKVLESGVNVTVSQFTLL | 1620 |
| QUERY: | 2617 | VNGRTRRFTNIEFQYSTLLLSIRYGLTPDTLDEEKARVLDQARQRALGTAWAKEQQKARD | 2676 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 1621 | VNGRTRRFTNIEFQYSTLLLSIRYGLTPDTLDEEKARVLDQARQRALGTAWAKEQQKARD | 1680 |
| QUERY: | 2677 | GREGSRLWTEGEKQQLLSTGRVQGYEGYYVLPVEQYPELADSSSNIQFLRQNEMGKR | 2733 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 1681 | GREGSRLWTEGEKQQLLSTGRVQGYEGYYVLPVEQYPELADSSSNIQFLRQNEMGKR | 1737 |

The amino acid sequences of the FCTR3bcde and f proteins were also found to have 2528 of 2774 amino acid residues (91%) identical to, and 2557 of 2774 residues (92%) positive with, the 2765 amino acid residue protein neurestin alpha [Rattus norvegicus] (GenBank Acc:AF086607) (SEQ ID NO:72), shown in Table 3T.

TABLE 3T

BLASTP of FCTR3bcd and f against Rattus norvegicus Neurestin alpha
(SEQ ID NO:72)

>GI|9910320|REF|NP 064473.1| NEUXESTIN ALPHA [RATTUS NORVEGICUS]
GI|5712201|GB|AAD47383.1|AF086607 1 (AF086607) NEURESTIN ALPHA [RATTUS NORVEGICUS]
LENGTH = 2765
SCORE = 4988 BITS (12938), EXPECT = 0.0
IDENTITIES = 2528/2774 (91%), POSITIVES = 2557/2774 (92%), GAPS = 50/2774 (1%)

| | | | |
|---|---|---|---|
| QUERY: | 1 | NDVKDRRHRSLTRGRCGKECRYTSSSLDSEDCRVPTQKSYSSSETLKAYDHDSPNHYGNR | 60 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 1 | MDVKDRRHRSLTRGRCGKECRYTSSSLDSHDCRVPTQKSYSSSETLKAYDHDSRMHYGNR | 60 |
| QUERY: | 61 | VTDLIHRESDEFPRQGTNFTLAELGICEPSPHRSGYCSDMGILEQGYSLSTGSDADSDTE | 120 |
| | | \|\|\|\|+\|\|\|\|\|\|\| \|\|\| \|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 61 | VTDLVHRESDEFSRQGANFTLAELGICEPSPHRSGYCSDMGILHQGYSLSTGSDADSDTE | 120 |
| QUERY: | 121 | GGMSPEHAIRLWGRGIKSRRSSGLSSRENSALTLTXXXXXXXXXXXXGRYXXXXXXXXXXX | 180 |
| | | \|\|\|\|\|\|\|\|\|\|\|\|\|\|\|\|\|\|\|\|\|\|\|\|\|\|\|\|\|\|\|\|\|\|\| \|\| | |
| SBJCT: | 121 | GGMSPEHAIRLWGRGIKSRRSSGLSSRENSALTLTDSDNENKSDDDNGRPIPPTSSSSLL | 180 |
| QUERY: | 181 | XXXXXXXXHNPPPVSCQMPLLDSNTSHQIMDTNPDEEFSPNSYLLRACXXXXXXXXXXXX | 240 |
| | | \|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 181 | PSAQLPSSHNPPPVSCQMPLLDSNTSHQIMDTNPDEEFSPNSYLLRACSGPQQASSSGPP | 240 |
| QUERY: | 241 | NHHSQXXXXXXXXXXXXXXXXXXXXXXXXXXXXXXXXXXXQIHAPAPAPNDLATTPESVQ | 300 |
| | | \|\|\|\|\| \|\|\|\|\|\|\|\|\|\|\|\|\|\|\|\|\|\|\|\| | |
| SBJCT: | 241 | NHHSQSTLRPPLPPPHNHTLSHHHSSANSLNPNSLTNRRSQIHAPAPAPNDLATTPESVQ | 300 |
| QUERY: | 301 | LQDSWVLNSNVPLETREFLFKXXXXXXXXXXXXXXXYPLTSGTVYTPPPRLLPRNTFSRK | 360 |
| | | \|\|\|\|\|\|\|\|\|\|\|\|\|\|\|\|\|\|\|\|\| \|\|\|\|\|\|\|\|\|\|\|\|\|\|\|\|\|\|\|\|\|\|\|\| | |
| SBJCT: | 301 | LQDSWVLNSNVPLETRHRLFKTSSGSTPLFSSSSPGYPLTSGTVYTPPPRLLPRNTESRK | 360 |
| QUERY: | 361 | AFKLKKPSKYCSWKCXXXXXXXXXXXXXXXXXYFI------------------------- | 395 |
| | | \|\|\|\|\|\|\|\|\|\|\|\|\|\|\| \|\|\| | |
| SBJCT: | 361 | AFKLKKPSKYCSWXCAALSAIAAALLLAILLAYFIAMHLLGLNWQLQPDGHTFNNGVRT | 420 |
| QUERY: | 396 | ----------------VPWSLKNSSIDSGEAEVGRRVTQEVPPGVFWRSQIHISQPQFLK | 439 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 421 | GLPGNDDVATVPSGGKVPWSLKNSSIDSGEAEVGRRVTQEVPPGVFWRSQIHISQPQFLK | 480 |
| QUERY: | 440 | FNISLGKDALFGVYIRRGLPPSHAQYDFMERLDGKEKWSVVESPRERRSIQTLVQNEAVF | 499 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 481 | FNISLGKDALFGVYIRRGLPPSHAQYDFMERLDGKEKWSVVESPRERRSIQTLVQNEAVF | 540 |
| QUERY: | 500 | VQYLDVGLWHLAFYNDGKDKEMVSFNTVVLDSVQDCPRNCHGNGECVSGVCHCFPGFLGA | 559 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|+\|\|\|\|\|\|\|\|\|\| | |
| SBJCT: | 541 | VQYLDVGLWHLAFYNDGKDKEMVSFNTVVLDSVQDCPRNCHGNGECVSGLCHCFPGFLGA | 600 |
| QUERY: | 560 | DCAKAACPVLCSGNGQYSKGTCQCYSGWKGAECDVPMNQCIDPSCGGHGSCIDGNCVCSA | 619 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|+\| | |
| SBJCT: | 601 | DCAKAACPVLCSGNGQYSKGTCQCYSGWKGAECDVPMNQCIDPSCGGHGSCIDGNCVCAA | 660 |
| QUERY: | 620 | GYKGEHCEEVDCLDPTCSSHGVCVNGECLCSPGWGGLNCELARVQCPDQCSGHGTYLPDT | 679 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|+ | |
| SBJCT: | 661 | GYKGEHCEEVDCLDPTCSSHGVCVNGECLCSPGWGGLNCELARVQCPDQCSGHGTYLPDS | 720 |
| QUERY: | 680 | GLCSCDPNWMGPDCSVEVCSVDCGTHGVCIGGACRCEEGWTGAACDQRVCHPRCIEHGTC | 739 |
| | | \|\|\|+\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 721 | GLCNCDPNWMGPDCSVEVCSVDCGTHGVCIGGACRCEEGWTGAACDQRVCHPRCIERGTC | 780 |
| | | ********* | |
| QUERY: | 740 | KDGKCECREGWNGEHCTIGRQTAGTETDGCPDLCNGNGRCTLGQNSWQCVCQTGWRGPGC | 799 |
| | | \|\|\|\|\|\|\|\|\|\|\|\|\|\|\|\|\|\| \|\|\|\|\|\|\|\|\|\|\|\|\|\|\|\|\|\|\|\|\|\|\|\|\|\|\|\|\|\|\|\|\| | |
| SBJCT: | 781 | KDGKCECREGWNGEHCTI---------DGCPDLCNGNGRCTLGQNSWQCVCQTGWRGPGC | 831 |
| QUERY: | 800 | NVAMETSCADNKDNEGDGLVDCLDPDCCLQSACQNSLLCRGSRDPLDIIQQGQTDWPAVK | 859 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 832 | NVAMETSCADNKDNEGDGLVDCLDPDCCLQSACQNSLLCRGSRDPLDIIQQGQTDWPAVK | 891 |
| QUERY: | 860 | SFYDRIKLLAGKDSTHIIPGENPFNSSLVSLIRGQVVTTDGTPLVGVNVSFVKYPRYGYT | 919 |
| | | \|\|\|\|\|\|\|\|\|\|\|\|\|\|\|\|\|\|\|\|+\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 892 | SFYDRIKLLAGICDSTHIIPGDNPFNSSLVSLIRGQVVTTDGTPLVGVNVSFVKYPKYGYT | 951 |
| QUERY: | 920 | ITRQDGTFDLIANGGASLTLHFERAPFMSQERTVWLPPWNSFYAMDTLVMKTEENSIPSCD | 979 |
| | | \|\|\|\|\|\|\|\|\|\|\|\|\|\|\|++\|\|\|\|\|\|\|\|\|\|\|\|+\|\|\|\|\| \|\|\|\|\|\|\|\|\|\|\|\|\|\|\|\|\|\|\|\|\|\|\|\| | |
| SBJCT: | 952 | ITRQDGTFDLIANGGSALTLHFERAPFMSRERTVWPPWNSFYAMDTLVMKTEENSIPSCD | 1011 |
| QUERY: | 980 | LSGFVRPDPIIISSFLSTFFSAAPGQNPIVPETQVLHEEIELPGSNVKLRYLSSRTAGYK | 1039 |
| | | \|\|\|\|\|\|\|\|\|\|\|\|\|\|\|\|\|\|\|\|\|\|+\| \|\|\|\|\|\|\|\|\|\|\|\|\|\|\|\|\|\|+\|\|\|\|\|\|\|\|\|\|\|\|\|\|\| | |
| SBJCT: | 1012 | LSGFVRPDPIIISSPLSTFFSASPAANPIVPETQVLHEEIELPGTNVKLRYLSSRTAGYK | 1071 |
| QUERY: | 1040 | SLLKITMTQSTVPLNLIRVHLMVAVEGHLFQKSFQASPNLASTFIWDKTDAYGQRVYGLS | 1099 |
| | | \|\|\|\|\|\|\|\|\|\|\|\|\|\|\|\|\|\|\|\|\|\|\|\|\|\|\|\|\|\|\|\|\|\|\|\|\|\|\|\|\| \|\|\|\|\|\|\|\|\|\|\|\|\|\|\|\|\|\| | |
| SBJCT: | 1072 | SLLKITMTQSTVPLNLIRVHLMVAVEGHLFQKSFQASPNLAYTFIWDKTDAYGQRVYGLS | 1131 |
| QUERY: | 1100 | DAVVSVGFEYETCPSLILWEKRTALLQGFELDPSNLGGWSLDKHHILNVKSGILHKGTGE | 1159 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\| \|\|\|\|\|\|\|\| \|\|\|\|\| | |
| SBJCT: | 1132 | DAVVSVGFEYETCPSLILWEKRTALLQGFELDPSNLGGWSLDKHHTLNVKSGILLKGTGE | 1191 |
| QUERY: | 1160 | NQFLTQQPAIITSIMGNGRRRSISCPSCNGLAEGNKLLAPVALAVGIDGSLYVGDFNYIR | 1219 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|+\|\|\|\|\|\|\|\| | |
| SBJCT: | 1192 | NQFLTQQPAIITSIMGNGRRRSISCPSCNGLAEGNKLLAPVALAVGIDGSLFVGDFNYIR | 1251 |
| QUERY: | 1220 | RIFPSRNVTSILELRNKEFRHSNNPAHKYYLAVDPVSGSLYVSDTNSRRIYRVKSLSGTK | 1279 |
| | | \|\|\|\|\|\|\|\|\|\|\|\|\|\|\|\|\|\|\|\|\|\|\|+\| \|\|\|\|\|\|\|\|\|\|+\|\|\|\|\|\|\|\|\|\|\|\|\|\|\|\|\|\|\|\|\| \| | |
| SBJCT: | 1252 | RIFPSRNVTSILELRNKEFKHSNSPGHKYYLAVDPVTGSLYVSDTNSRRIYRVKSLSGAK | 1311 |
| QUERY: | 1280 | DLAGNSEVVAGTGEQCLPFDEARCGDGGKAIDATLMSPRGIAVDKNGLMYFVDATMIRKV | 1339 |
| | | \|\|\|\|\|\|\|\|\|\|\|\|\|\|\|\|\|\|\|\|\|\|\|\|\|\|\|\|\|\|+\|\|\|\|\|\|\|\|\|\|\|\|\|\|\|\|\|\|\|\|\|\|\|\|\|\|\|\|\| | |
| SBJCT: | 1312 | DLAGNSEVVAGTGEQCLPFDEARCGDGGKAVDATLMSPRGIAVDKNGLMYFVDATMIRKV | 1371 |
| QUERY: | 1340 | DQNGIISTLLGSNDLTAVRPLSCDSSMDVAQVRLEWPTDLAVNPMDNSLYVLENNVILRI | 1399 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 1372 | DQNGIISTLLGSNDLTAVRPLSCDSSMDVAQVRLEWPTDLAVNPMDNSLYVLENNVILRI | 1431 |
| QUERY: | 1400 | TENHQVSIIAGRPMHCQVPGIDYSLSKXXXXXXXXXXXXXXXXXTGVLYITETDEKKINR | 1459 |
| | | \|\|\|\|\|\|\|\|\|\|\|\|\|\|\|\|\|\|\|\|\|\|\|\|\|\|\| \|\|\|\|\|\|\|\|\|\|\|\|\|\|\|\| | |
| SBJCT: | 1432 | TRNRQVSIIAGRPMHCQVPGIDYSLSKLAIHSALESASAIAISHTGVLYITETDEKKINR | 1491 |
| QUERY: | 1460 | LRQVTTNGEICLLAGAASXXXXXXXXXXXXYSGDDAYATDAILNSPSSLAVAPDGTIYIA | 1519 |
| | | \|\|\|\|\|\|\|\|\|\|\|\|\|\|\|\|\|\| \|\|\|\|\|\|\|\|\|\|\|\|\|\|\|\|\|\|\|\|\|\|\|\|\|\|\|\|\|\|\| | |
| SBJCT: | 1492 | LRQVTTNGEICLLAGAASDCDCKNDVNCICYSGDDAYATDAILNSPSSLAVAPDGTIYIA | 1551 |
| QUERY: | 1520 | DLGNIRIRAVSKNKPVLNAFNQYEAASPGEQELYVFNADGIHQYTVSLVTGEYLYNFTYS | 1579 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 1552 | DLGNIRIRAVSKNKPVLNAFNQYEAASPGEQELYVFNADGIHQYTVSLVTGEYLYNFTYS | 1611 |
| QUERY: | 1580 | TDNDVTELIDNNGNSLKIRRDSSGMPRHLLMPDNQIITLTVGTNGGLKVVSTQNLELGLM | 1639 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| \|\|\|\|\|\|\|\|\|\|\| | |
| SBJCT: | 1612 | ADNDVTELIDNNGNSLKIRRDSSGMPRRLLMPDNQIITLTVGTNGGLKAVSTQNLELGLM | 1671 |
| QUERY: | 1640 | TYDGNTGLLATKSDETGWTTFYDYDHEGRLTNVTRPTGVVTSLHREMEKSITIDIENSNR | 1699 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|+\|\|\|\|\|\|\| | |
| SBJCT: | 1672 | TYDGNTGLLATKSDETGWTTFYDYDEEGRLTNVTRPTGVVTSLHREMEKSITVDIENSNR | 1731 |
| QUERY: | 1700 | DDDVTVITNLSSVEASYTVVQDQVRNSYQLCNNGTLRVMYANGMGISFHSEPHVLAGTIT | 1759 |
| | | \|+\|\|\|\|\|\|\|\|\|\|\|\|\|\|\|\|\|\|\|\|\|\|\|\|\|\|\|\|\|+\|\|\|\|\|\|\|\|\|\|\|\|\|+\|\|\|\|\|\|\|\|\|\|\|\|+\| | |
| SBJCT: | 1732 | DNDVTVITNLSSVEASYTVVQDQVRNSYQLCSNGTLRVMYANGMGVSFHSEPHVLAGTLT | 1791 |
| QUERY: | 1760 | PTIGRCNISLPMENGLNSIEWRLRKEQIKGKVTIFGRKLRVHGRNLLSIDYDRNIRTEKI | 1819 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 1792 | PTIGRCNISLPMENGLNSIEWRLRKEQIKGKVTIFGRKLRVHGRNLLSIDYDRNIRTEKI | 1851 |
| QUERY: | 1820 | YDDHRKFTLRIIYDQVGRPFLWLPSSGLAAVNVSYFFNGRLAGLQRGAMSERTDIDKQGR | 1879 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 1852 | YDDHRKFTLRIIYDQVGRPFLWLPSSGLAAVNVSYFFNGRLAGLQRGAMSERTDIDKQGR | 1911 |
| QUERY: | 1880 | IVSRMFADGKVWSYSYLDKSMVLLLQSQRQYIFEYDSSDRLLAVTMPSVARHSMSTHTSI | 1939 |
| | | \|\|\|\|\|\|\|\|\|\|\|\|\|\|\|\|\|\|\|\|\|\|\|\|\|\|\|\|\|\|\|\|\|\|\|\|\|\|\|\|\| \|\|\|\|\|\|\|\|\|\|\|\|\|\|\|\|\|\| | |
| SBJCT: | 1912 | IVSRMFADGKVWSYSYLDKSMVLLLQSQRQYIFEYDSSDRLHAVTMPSVARHSMSTHTSI | 1971 |
| QUERY: | 1940 | GYIRNIYNPPESNASVIFDYSDDGRILKTSFLGTGRQVFYKYGKLSKLSEIVYDSTAVTF | 1999 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 1972 | GYIRNIYNPPESNASVIFDYSDDCRILKTSFLGTGRQVFYKYGKLSKLSEIVYDSTAVTF | 2031 |
| QUERY: | 2000 | GYDETTGVLKMVNLQSGGFSCTIRYRKIGPLVDKQIYRFSEEGMVNARFDYTYHDNSFRI | 2059 |
| | | \|\|\|\|\|\|\|\|\|\|\|\|\|\|\|\|\|\|\|\|\|\|\|\|\|\|\|+\|\|\|\|\|\|\|\|\|\|\|\|\|\|\|\|+\|\|\|\|\|\|\|\|\|\|\|\|\|\|\| | |
| SBJCT: | 2032 | GYDETTGVLKMVNLQSGGFSCTIRYRKVGPLVDKQIYRFSEEGMINARFDYTYRDNSFRI | 2091 |
| QUERY: | 2060 | ASIKFVISETPLPVDLYRYDEISGKVEHFGKFGVIYYDINQIITTAVMTLSKHFDTHGRI | 2119 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 2092 | ASIKPVISETPLPVDLYRYDEISGKVEHFGKFGVIYYDINQIITTAVNTLSKHFDTHGRI | 2151 |
| QUERY: | 2120 | KEVQYEMFRSLMYWMTVQYDSMGRVIKRELKLGPYANTTKYTYDYDGDGQLQSVAVNDRP | 2179 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 2152 | KEVQYEMFRSLMYWMTVQYDSMGRVIKRELKLGPYANTThYTYDYDGDGQLQSVAVNDRP | 2211 |
| QUERY: | 2180 | TWRYSYDXXXXXXXXXXXXSVRLMPLRYDLRDRITRLGDVQYKIDDDGYLCQRGSDIFEY | 2239 |
| | | \|\|\|\|\|\|\| \| \|\|\|\|\|\|\|\|\|\|\|\|\|\|\|\|\|\|\|\|\|\|\|\|\|\|\|\|\|\|\|\|\|\|\|\|\|\|\| | |
| SBJCT: | 2212 | TWRYSYDLNGNLHLLNPGNSARLMPLRYDLRDRITRLGDVQYKIDDDGYLCQRGSDIFEY | 2271 |
| QUERY: | 2240 | NSKGLLTRAYNKASGWSVQYRYDGVGRRASYKTNLGHHLQYFYSDLHNPTRITHVYNHSN | 2299 |
| | | \|\|\|\|\|\|\|\|\|\|\|\|\|\|\|\|\|\|\|\|\|\|\|\|\| \|\|\|\|\|\|\|\|\|\|\|\|\|\|\|\|\|\|\|\|\|+\|\|\|\|\|\|\|\|\|\|\|\| | |
| SBJCT: | 2272 | NSKGLLTRAYNKASGWSVQYRYDGVSRRASYKTNLGHRLQYFYSDLHHPTRITHVYNHSN | 2331 |
| QUERY: | 2300 | SEITSLYYDLQGRLFAMESSSGEEYYVASDNTGTPLAVFSINGLMIKQLQYTAYGEIYYD | 2359 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 2332 | SEITSLYYDLQGHLPAMESSSGEEYYVASDNTGTPLAVFSINGLMIKQLQYTAYGEIYYD | 2391 |
| QUERY: | 2360 | SNPDFQMVIGFHGGLYDPLTKLVHFTQRDYDVLAGRWTSPDYTNWKNVGKEPAPFNLYMF | 2419 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|+\|\|\|\|\|\|\|\|\|\|\|\|\|\| | |
| SBJCT: | 2392 | SNPDFQMVIGFHGGLYDPLTKLVHFTQRDYDVLAGRWTSPDYTMWRNVGKEPAPFNLYMF | 2451 |
| QUERY: | 2420 | KSNNPLSSELDLKNYVTDVKSWLVMFGFQLSNIIPGFPRAKMYFVPPPYELSESQASENG | 2479 |
| | | \|+\|\|\|\|\|+\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 2452 | KNNNPLSNELDLKNYVTDVKSWLVMFGFQLSNIIPGFPRAKMYFVPPPYELSESQASENG | 2511 |
| QUERY: | 2480 | QLITGVQQTTERHNQAFMALEGQVITKKLHASIREKAGHWFATTTPIIGKGIMFAIKEGR | 2539 |
| | | \|\|\|\|\|\|\|\|\|\|\|\|\|\|\|\|\|+\|\|\|\|\|\|\|+\|\|\|\|\| \|\|\|\|\|\|\|\|\|\|\|\|\|\|\|\|\|\|\|\|\|\|\|\|\|\|\|\| | |
| SBJCT: | 2512 | QLITGVQQTTERHNQAFLALEGQVISKKLHAGIREKAGHWFATTTPIIGKGIMFAIKEGR | 2571 |
| QUERY: | 2540 | VTTGVSSIASEDSRKVASVLNNAYYLDKNRYSIEGKDTHYFVKIGSADGDLVTLGTTIGR | 2599 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|+\|\|\|\|\|\|\|\|\|\|\|\|\|\| | |
| SBJCT: | 2572 | VTTGVSSIASEDSRKVASVLNNAYYLDKMHYSIEGKDTHYFVKIGAADGDLVTLGTTIGR | 2631 |
| QUERY: | 2600 | KVLESGVNVTVSQPTLLVNGRTRRFTNIEFQYSTLLLSIRYGLTPDTLDEEKARVLDQAR | 2659 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 2632 | KVLESGVNVTVSQPTLLVNGRTRRFTNIEFQYSTLLLSIRYGLTPDTLDEEKARVLDQAR | 2691 |
| QUERY: | 2660 | QRALGTAWAKEQQKARDGREGSRLWTEGEKQQLLSTGRVQGYEGYYVLPVEQYPELADSS | 2719 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 2692 | QRALGTAWAKEQQKARDGREGSRLWTEOERQQLLSTGRVQGYEGYYVLPVEQYPELADSS | 2751 |
| QUERY: | 2720 | SNIQFLRQNEMGKR 2733 | |
| | | \|\|\|\|\|\|\|\|\|\|\|\|\|\| | |
| SBJCT: | 2752 | SNIQFLRQNEMGKR 2765 | |

* = FCTR3F DOES NOT CONTAIN THESE AMINO ACIDS

The amino acid sequences of the FCTR3bcde and f proteins were also found to have 2536 of 2774 amino acid residues (91%) identical to, and 2558 of 2774 residues (91%) positive with, the 2764 amino acid residue protein Odd Oz/ten-m homolog 2 (Drosophila) (GenBank Acc:NP035986.2) (SEQ ID NO:65), shown in Table 3U.

TABLE 3U

BLASTP of FCTR3bcde and f against Odd Oz/ten-m homolog 2
(SEQ ID NO:65)

>GI|7657415|REF|NP 035986.2| ODD OZ/TEN-M HOMOLOG 2 (DROSOPHILA); ODD OZ/TEN-M
HOMOLOG 3
(DROSOPHILA) [MUS MUSCULUS]
GI|4760778|DBJ|BAA77397.1| (AB025411) TEN-M2 [MUS MUSCULUS]
LENGTH = 2764
SCORE = 4996 BITS (12961), EXPECT = 0.0
IDENTITIES = 2536/2774 (91%), POSITIVES = 2558/2774 (91%), GAPS = 51/2774 (1%)

| | | | |
|---|---|---|---|
| QUERY: | 1 | MDVKDRRHRSLTRGRCGKECRYTSSSLDSEDCRVPTQKSYSSSETLKAYDHDSRNHYGNR | 60 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 1 | MDVKDRRHRSLTRGRCGKECRYTSSSLDSEDCRVPTQKSYSSSETLKAYDHDSRMEYGNR | 60 |
| QUERY: | 61 | VTDLIHRESDEFPRQGTNFTLAELGICEPSPHRSGYCSDMGILHQGYSLSTGSDADSDTE | 120 |
| | | \|\|\|\|+\|\|\|\|\|\|\| \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 61 | VTDLVHRESDEFSRQGTNFTLAELGICEPSPERSGYCSDMGILHQGYSLSTGSDADSDTE | 120 |
| QUERY: | 121 | GGMSPEHAIRLWGRGIKSRRSSGLSSRENSALTLTXXXXXXXXXXXXGRXXXXXXXXXXX | 180 |
| | | \|\|\|\|\|\|\|\|\|\|\|\|\|\|\|\|\|\|\|\|\|\|\|\|\|\|\|\|\|\|\|\|\|\|\| \|\| | |
| SBJCT: | 121 | GGMSPEHAIRLWGRGIKSRRSSGLSSRENSALTLTDSDNENKSDDDNGRPIPPTSSSSLL | 180 |
| QUERY: | 181 | XXXXXXXXHNPPPVSCQMPLLDSNTSHQIMDTNPDEEFSPNSYLLRACXXXXXXXXXXXX | 240 |
| | | \|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 181 | PSAQLPSSHNPPPVSCQMPLLDSNTSHQIMDTNPDEEFSPNSYLLRACSGPQQASSSGPP | 240 |
| QUERY: | 241 | NRHSQXXXXXXXXXXXXXXXXXXXXXXXXXXXXXXXXXXXQIHAPAPAPNDLATTPESVQ | 300 |
| | | \|\|\|\|\| \|\|\|\|\|\|\|\|\|\|\|\|\|\|\|\|\|\|\|\| | |
| SBJCT: | 241 | NHHSQSTLRPPLPPPHNHTLSHHHSSANSLNRNSLTNRRSQIHAPAPAPNDLATTPESVQ | 300 |
| QUERY: | 301 | LQDSWVLNSNVPLETRHFLFEXXXXXXXXXXXXXXXYPLTSGTVYTPPPRLLPRNTFSRK | 360 |
| | | \|\|\|\|\|\|\|\|\|\|\|\|\|\|\|\|\|\|\|\|\| \|\|\|\|\|\|\|\|\|\|\|\|\|\|\|\|\|\|\|\|\|\|\|\| | |
| SBJCT: | 301 | LQDSWVLNSNVPLETRHFLFKTSSGSTPLFSSSSPGYPLTSGTVYTPPPRLLPRNTFSRK | 360 |
| QUERY: | 361 | AFKLKKPSKYCSWRCXXXXXXXXXXXXXXXXXYFI------------------------- | 395 |
| | | \|\|\|\|\|\|\|\|\|\|\|\|\|\|\| \|\|\| | |
| SBJCT: | 361 | AFKLKKPSKYCSWKCAALSAIAAALLLAILLAYFIAMHLLGLNWQLQPADGHTFNNGVRT | 420 |
| QUERY: | 396 | ----------------VPWSLKNSSIDSGEAEVGRRVTQEVPPGVFWRSQIHISQPQFLK | 439 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 421 | GLPGNDDVATVPSGGKVPWSLKNSSIDSGEAEVGRRVTQEVPPGVFWRSQIHISQPQFLK | 480 |
| QUERY: | 440 | FNISLGKDALFGVYIRRGLPPSHAQYDFMERLDGEEKWSVVESPRERRSIQTLVQNEAVF | 499 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 481 | FNISLGKDALFGVYIRRGLPPSHAQYDFMERLDGKEKWSVVESPRERRSIQTLVQNEAVF | 540 |
| QUERY: | 500 | VQYLDVGLWHLAFYNDGKDKEMVSFNTVVLDSVQDCPRNCHGNGECVSGVCHCFPGFLGA | 559 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|+\|\|\|\|\|\|\|\|\|\| | |
| SBJCT: | 541 | VQYLDVGLWHLAFYNDGEDKEMVSFNTVVLDSVQDCPRNCHGNGECVSGLCHCFPGFLGA | 600 |
| QUERY: | 560 | DCAKAACPVLCSGNGQYSKGTCQCYSGWKGAECDVPNNQCIDPSCGGHGSCIDGNCVCSA | 619 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|+\| | |
| SBJCT: | 601 | DCAKAACPVLCSGNGQYSKGTCQCYSGWKGAECDVPMNQCIDPSCGGHGSCIDGNCVCAA | 660 |
| QUERY: | 620 | GYKGEHCEEVDCLDPTCSSHGVCVNGECLCSPGWGGLNCELARVQCPDQCSGHGTYLPDT | 679 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|+ | |
| SBJCT: | 661 | GYKGEHCEEVDCLDPTCSSHGVCVNGECLCSPGWGGLNCELARVQCPDQCSGHGTYLPDS | 720 |
| QUERY: | 680 | GLCSCDPNWNGPDCSVEVCSVDCGTEGVCIGGACRCEEGWTGAACDQRVCHPRCIEHGTC | 739 |
| | | \|\|\|\|\|\|\|\|\|\|\|\|\|\|\|\| \|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 721 | GLCSCDPNNMGPDCSV-VCSVDCGTHGVCIGGACRCEEGWTGAACDQRVCHPRCIEHGTC | 779 |
| | | ********* | |
| QUERY: | 740 | KDGKCECREGWNGEHCTIGRQTAGTETDGCPDLCNGNGRCTLGQNSWQCVCQTGWRGPGC | 799 |
| | | \|\|\|\|\|\|\|\|\|\|\|\|\|\|\|\|\|\| \|\|\|\|\|\|\|\|\|\|\|\|\|\|\|\|\|\|\|\|\|\|\|\|\|\|\|\|\|\|\|\|\| | |
| SBJCT: | 780 | KDGKCECREGWNGEHCTI---------DGCPDLCNGNGRCTLGQNSWQCVCQTGWRGPGC | 830 |
| QUERY: | 800 | NVAMETSCADNKDNEGDGLVDCLDPDCCLQSACQNSLLCRGSRDPLDIIQQGQTDWPAVK | 859 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 831 | NVANETSCADNKDNEGDGLVDCLDPDCCLQSACQNSLLCRGSRDPLDIIQQGQTDWPAVK | 890 |
| QUERY: | 860 | SFYDRIKLLAGKDSTHIIPGENPFNSSLVSLIRGQVVTTDGTPLVGVNVSFVKYPKYGYT | 919 |
| | | \|\|\|\|\|\|\|\|\|\|\|\|\|\|\|\|\|\|\|\|+\|\|\|\|\|\|\|\|\|\|\|\|\|\|\|\|\| \|\|\|\|\|\|\|\|\|\|\|\|\|\|\|\|\|\|\|\|\|\| | |
| SBJCT: | 891 | SFYDRIKLLAGKDSTHIIPGDNPFNSSLVSLIRGQVVTMDGTPLVGVNVSFVKYPKYGYT | 950 |
| QUERY: | 920 | ITRQDGTFDLIANGGASLTLHFERAPFMSQERTVWLPWNSFYAMDTLVMKTEENSIPSCD | 979 |
| | | \|\|\|\|\|\|\|\|\|\|\|\|\|\|\|++\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 951 | ITRQDGTFDLIANGGSALTLHFERAPFMSQERTVWLPWNSFYAMDTLVMKTEENSIPSCD | 1010 |
| QUERY: | 980 | LSGFVRPDPIIISSPLSTFFSAAPGQNPIVPETQVLHEEIELPGSNVKLRYLSSRTAGYK | 1039 |
| | | \|\|\|\|\|\|\|\|\|\|\|\|\|\|\|\|\|\|\|\|\|\|+\| \|\|\|\|\|\|\|\|\|\|\|\|\|\|\|\|\|\|+\|\|\|\|\|\|\|\|\|\|\|\|\|\|\| | |
| SBJCT: | 1011 | LSGFVRPDPIIISSPLSTFFSASPASNPIVPETQVLHEEIELPGTNVKLRYLSSRTAGYK | 1070 |
| QUERY: | 1040 | SLLKITMTQSTVPLNLIRVHLMVAVEGHLFQKSFQASPNLASTFIWDKTDAYGQRVYGLS | 1099 |
| | | \|\|\|\|\|\|\|\|\|\|\|\|\|\|\|\|\|\|\|\|\|\|\|\|\|\|\|\|\|\|\|\|\|\|\|\|\|\|\|\|\| \|\|\|\|\|\|\|\|\|\|\|\|\|\|\|\|\|\| | |
| SBJCT: | 1071 | SLLKITMTQSTVPLNLIRVHLMVAVEGHLFQKSFQASPNLAYTFIWDKTDAYGQRVYGLS | 1130 |
| QUERY: | 1100 | DAVVSVGFEYETCPSLILWEKRTALLQGFELDPSNLGGWSLDKHHILNVKSGILHKGTGE | 1159 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\| \|\|\|\|\|\|\|\|\|\|\|\|\|\| | |
| SBJCT: | 1131 | DAVVSVGFEYETCPSLILWEKRTALLQGFELDPSNLGGWSLDKHHTLNVKSGILHKGTGE | 1190 |
| QUERY: | 1160 | NQFLTQQPAIITSIMGNGRRRSISCPSCNGLAEGNKLLAPVALAVGIDGSLYVGDFNYIR | 1219 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|+\|\|\|\|\|\|\|\| | |
| SBJCT: | 1191 | NQFLTQQPAIITSIMGNGRRRSISCPSCNGLAEGNKLLAPVALAVGIDGSLFVGDFNYIR | 1250 |
| QUERY: | 1220 | RIFPSRNVTSILELRNXEFKHSNNPAHKYYLAVDPVSGSLYVSDTNSRRIYRVKSLSGTK | 1279 |
| | | \|\|\|\|\|\|\|\|\|\|\|\|\|\|\|\|\|\|\|\|\|\|\|+\| \|\|\|\|\|\|\|\|\|\|+\|\|\|\|\|\|\|\|\|\|\|\|\|\|\|\|\|\|\|\|\| \| | |
| SBJCT: | 1251 | RIFPSRNVTSILELRNKEFKHSNSPGHKYYLAVDPVTGSLYVSDTNSRRIYRVKSLSGAK | 1310 |
| QUERY: | 1280 | DLAGNSEVVAGTGEQCLPFDEARCGDGGKAIDATLMSPRGIAVDKNGLMYFVDATMIRKV | 1339 |
| | | \|\|\|\|\|\|\|\|\|\|\|\|\|\|\|\|\|\|\|\|\|\|\|\|\|\|\|\|\|\|+\|\|\|\|\|\|\|\|\|\|\|\|\|\|\|\|\|\|\|\|\|\|\|\|\|\|\|\|\| | |
| SBJCT: | 1311 | DLAGNSEVVAGTGEQCLPFDEARCGDGGKAVDATLMSPRGIAVDKNGLMYFVDATMIRKV | 1370 |
| QUERY: | 1340 | DQNGIISTLLGSNDLTAVRPLSCDSSMDVAQVRLEWPTDLAVNPMDNSLYVLENNVILRI | 1399 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 1371 | DQNGIISTLLGSNDLTAVRPLSCDSSMDVAQVRLEWPTDLAVNPMDNSLYVLENNVILRI | 1430 |
| QUERY: | 1400 | TENHQVSIIAGRPNHCQVPGIDYSLSKXXXXXXXXXXXXXXXXXTGVLYITETDEKRINR | 1459 |
| | | \|\|\|\|\|\|\|\|\|\|\|\|\|\|\|\|\|\|\|\|\|\|\|\|\|\|\| \|\|\|\|\|\|\|\|\|\|\|\|\|\|\|\| | |
| SBJCT: | 1431 | TENHQVSIIAGRPMHCQVPGIDYSLSRLAIHSALESASAIAISHTGVLYITETDEKKINR | 1490 |
| QUERY: | 1460 | LRQVTTNGEICLLAGAASXXXXXXXXXXXXYSGDDAYATDAILNSPSSLAVAPDGTIYIA | 1519 |
| | | \|\|\|\|\|\|\|\|\|\|\|\|\|\|\|\|\|\| \|\|\|\|\|\|\|\|\|\|\|\|\|\|\|\|\|\|\|\|\|\|\|\|\|\|\|\|\|\| | |
| SBJCT: | 1491 | LRQVTTNGEICLLAGAASDCDCKNDVNCICYSGDDAYATDAILNSPSSLAVAPDGTIYIA | 1550 |
| QUERY: | 1520 | DLGNIRIRAVSKNKPVLNAFNQYEAASPGEQELYVFNADGIHQYTVSLVTGEYLYNFTYS | 1579 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 1551 | DLGNIRIRAVSKNKPVLNAFNQYEAASPGEQELYVFNADGIHQYTVSLVTGEYLYNFTYS | 1610 |
| QUERY: | 1580 | TDNDVTELIDNNGNSLKIRRDSSGMPRHLLMPDNQIITLTVGTNGGLKVVSTQNLELGLM | 1639 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| \|\|\|\|\|\|\|\|\|\|\| | |
| SBJCT: | 1611 | ADNDVTELIDNNGNSLKIRRDSSGMPRHLLMPDNQIITLTVGTNGGLKAVSTQNLELGLM | 1670 |
| QUERY: | 1640 | TYDGNTGLLATKSDETGWTTFYDYDHEGRLTNVTRPTGVVTSLHREMEKSITIDIENSNR | 1699 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 1671 | TYDGNTGLLATKSDETGWTTFYDYDHEGRLTNVTRPTGVVTSLHREMERSITIDIENSNR | 1730 |
| QUERY: | 1700 | DDDVTVITNLSSVEASYTVVQDQVRNSYQLCNNGTLRVMYANGMGISFHSEPHVLAGTIT | 1759 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\| +\|\|\|\|\|\|\|\|\|\|\|\|\|\| | |
| SBJCT: | 1731 | DDDVTVITNLSSVEASYTVVQDQVRNSYQLCNNGTLRVMYANGMAVSFHSEPHVLAGTIT | 1790 |
| QUERY: | 1760 | PTIGRCNISLPMENGLNSIEWRLRKEQIKGKVTIFGRKLRVHGRNLLSIDYDRNIRTEKI | 1819 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 1791 | PTIGRCNISLPMENGLNSIEWRLRKRQIKGKVTIFGRKLRVHGRNLLSIDYDRNIRTEKI | 1850 |
| QUERY: | 1820 | YDDHRKFTLRIIYDQVGRPFLWLPSSGLAAVNVSYFFNGRLAGLQRGAMSERTDIDKQGR | 1879 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 1851 | YDDHRRFTLRIIYDQVGRPFLWLPSSGLAAVNVSYFFNGRLAGLQRGAMSERTDIDRQGR | 1910 |
| QUERY: | 1880 | IVSRMFADGKVWSYSYLDKSMVLLLQSQRQYIFEYDSSDRLLAVTMPSVAPESMSTHTSI | 1939 |
| | | \|\|\|\|\|\|\|\|\|\|\|\|\|\|\|\|\|\|\|\|\|\|\|\|\|\|\|\|\|\|\|\|\|\|\|\|\|\|\|\|\| \|\|\|\|\|\|\|\|\|\|\|\|\|\|\|\|\|\| | |
| SBJCT: | 1911 | IVSRMFADGKVWSYSYLDRSMVLLLQSQRQYIFEYDSSDRLHAVTMPSVARHSMSTHTSI | 1970 |
| QUERY: | 1940 | GYIRNIYNPPESNASVIFDYSDDGRILRTSFLGTGRQVFYKYGKLSRLSEIVYDSTAVTF | 1999 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 1971 | GYIRNIYNPPESNASVIFDYSDDGRILKTSFLGTGRQVFYKYGRLSRLSEIVYDSTAVTF | 2030 |
| QUERY: | 2000 | GYDETTGVLKMVNLQSGGFSCTIRYRKIGPLVDKQIYRFSEEGMVNARFDYTYHDNSFRI | 2059 |
| | | \|\|\|\|\|\|\|\|\|\|\|\|\|\|\|\|\|\|\|\|\|\|\|\|\|\|\|+\|\|\|\|\|\|\|\|\|\|\|\|\|\|\|\|+\|\|\|\|\|\|\|\|\|\|\|\|\|\|\| | |
| SBJCT: | 2031 | GYDETTGVLKMVNLQSGGFSCTIRYRXVGPLVDKQIYRFSEEGMINARFDYTYHDNSFRI | 2090 |
| QUERY: | 2060 | ASIKPVISETPLPVDLYRYDEISGKVEHFGKFGVIYYDINQIITTAVNTLSKHFDTHGRI | 2119 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 2091 | ASIKPVISETPLPVDLYRYDEISGKVEHFGKFGVIYYDINQIITTAVMTLSKHFDTHGRI | 2150 |
| QUERY: | 2120 | KEVQYEMFRSLMYWMTVQYDSMQRVIKRELKLGPYANTTRYTYDYDCDGQLQSVAVNDRP | 2179 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 2151 | KEVQYEMFRSLMYWMTVQYDSMGRVIKRELKLGPYANTTKYTYDYDGDGQLQSVAVNDRP | 2210 |
| QUERY: | 2180 | TWRYSYDXXXXXXXXXXXXSVRLMPLRYDLRDRITRLGDVQYKIDDDGYLCQRGSDIFEY | 2239 |
| | | \|\|\|\|\|\|\| \| \|\|\|\|\|\|\|\|\|\|\|\|\|\|\|\|\|\|\|\|\|\|\|\|\|\|\|\|\|\|\|\|\|\|\|\|\|\|\| | |
| SBJCT: | 2211 | TWRYSYDLNGNLHLLNPGNSARLMPLRYDLRDRITRLGDVQYKIDDDGYLCQRGSDIFEY | 2270 |
| QUERY: | 2240 | NSKGLLTRAYNKASGWSVQYRYDGVGRRASYKTNLGHHLQYFYSDLHNPTRITHVYNHSN | 2299 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 2271 | NSKGLLTRAYNKASGWSVQYRYDGVGRRASYKTNLGHHLQYFYSDLHNPTRITHVYNHSN | 2330 |
| QUERY: | 2300 | SEITSLYYDLQGHLFANESSSGEEYYVASDNTGTPLAVFSINGLMIKQLQYTAYGEIYYD | 2359 |
| | | \|\|\|\|\|\|\|\|\|\|\|\|\|\|\|\|\|\|\|\|\|\|\|\|\|\|\|\|\|\|\|\|\|\|\|\|\|\|+\|\|\|\|\|\|\|\|\|\|\|\|\|\|\|\|\|\|\|\|\| | |
| SBJCT: | 2331 | SEITSLYYDLQGHLFAMESSSGEEYYVASDNTGTPLVYSINGLMIKQLQYTAYGEIYYD | 2390 |
| QUERY: | 2360 | SNPDFQMVIGFHGGLYDPLTKLVHFTQRDYDVLAGRWTSPDYTMWKNVGKEPAPFNLYMF | 2419 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|+\|\|\|\|\|\|\|\|\|\|\|\|\|\| | |
| SBJCT: | 2391 | SNPDFQMVIGFHGGLYDPLTKLVRFTQRDYDVLAGRWTSPDYTMWRNVGKEPAPFNLYMF | 2450 |
| QUERY: | 2420 | KSNNPLSSELDLKNYVTDVKSWLVMFGFQLSNIIPGFPRAKMYFVPPPYELSESQASENG | 2479 |
| | | \|+\|\|\|\|\|+\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 2451 | KNNNPLSNELDLKNYVTDVKSWLVMFGBQLSNIIPGFPRAKMYFVPPPYELSESQASENG | 2510 |
| QUERY: | 2480 | QLITGVQQTTERHMQAFMALEGQVITKKLHASIREKAGHWFATTTPIIGKGIMFAIKEGR | 2539 |
| | | \|\|\|\|\|\|\|\|\|\|\|\|\|\|\|\|\|+\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 2511 | QLITGVQQTTERHNQAFLALEGQVITKKLHASIREKAGHWFATTTPIIGKGIMFAIKEGR | 2570 |
| QUERY: | 2540 | VTTGVSSIASEDSRKVASVLNNAYYLDKMHYSIEGRDTHYFVKIGSADGDLVTLGTTIGR | 2599 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|+\|\|\|\|\|\|\|\|\|\|\|\|\|\| | |
| SBJCT: | 2571 | VTTGVSSIASEDSRKVASVLNNAYYLDKMHYSIEGKDTHYFVKIGAADGDLVTLGTTIGR | 2630 |
| QUERY: | 2600 | KVLESGVNVTVSQPTLLVNQRTRRFTNIEFQYSTLLLSIRYGLTPDTLDEEKARVLDQAR | 2659 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 2631 | KVLESGVNVTVSQPTLLVNGRTRRFTNIEFQYSTLLLSIRYGLTPDTLDEEKARVLDQAG | 2690 |
| QUERY: | 2660 | QRALGTAWAKEQQKARDGREGSRLWTEGEKQQLLSTGRVQGYRGYYVLPVEQYPELADSS | 2719 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: | 2691 | QRALGTAWAKEQQKARDGREGSRLWTEGEKQQLLSTGRVQGYEGYYVLPVEQYPELADSS | 2750 |
| QUERY: | 2720 | SNIQFLRQNEMGKR 2733 | |
| | | \|\|\|\|\|\|\|\|\|\|\|\|\|\| | |
| SBJCT: | 2751 | SNIQFLRQNEMGKR 2764 | |

* = FCTR3F DOES NOT CONTAIN THESE AMINO ACIDS

FCTR3 is related to rat neurestin, a gene implicated in neuronal development (Otaki J M, Firestein S Dev Biol 1999 Aug. 1;212(1):165-81) Neurestin shows homology to human gamma-heregulin, a Drosophila receptor-type pair-rule gene product, Odd Oz (Odz)/Ten(m), and Ten(a). Neurestin has putative roles in synapse formation and brain morphogenesis. A mouse neurestin homolog, DOC4, has independently been isolated from the NIH-3T3 fibroblasts. DOC4 is also known as tenascin M (TNM), a Drosophila pair-rule gene homolog containing extracellular EGF-like repeats. The significant homology to these molecules and in particular, γ-heregulin, have important implications regarding the potential contribution of FCTR3 to disease progression. Heregulin is the ligand for HER-2/ErbB2/NEU, a proto-oncogene receptor tyrosine kinase implicated in breast and prostate cancer progression that was originally identified in rat neuro/glioblastoma cell lines. Extopic expression of HER-2/ErbB2/NEU in MDA-MB-435 breast adenocarcinoma cells confers chemoresistance to Taxol-induced apoptosis relative to vector transfected control cells (Yu et al. Overexpression of ErbB2 blocks Taxol-induced apoptosis by up-regulation of p21Cipi, which inhibits p34Cdc2 kinase. Molec. Cell 2: 581-591, 1998).

FCTR3 related tenascins and cancer biology

As mentioned, FCTR3 also has significant homology to DOC4, (AKA tenascin M), a Drosophila pair-rule gene homolog containing extracellular EGF-like repeats. The tenascins are a growing family of extracellular matrix proteins that play prominent roles in tissue interactions critical to embryogenesis. Overexpression of tenascins has been described in multiple human solid malignancies.

The role of the tenascin family of related proteins is to regulate epithelial-stromal interactions, participate in fibronectin-dependent cell attachment and interaction. Indeed, tenascin-C (TN) is overexpressed in the stroma of malignant ovarian tumours particularly at the interface between epithelia and stroma leading to suggestions that it may be involved in the process of invasion (Wilson et al (1996) Br J Cancer 74: 999-1004). Tenascin-C is considered a therapeutic target for certain malignant brain tumors (Gladson CL: J Neuropathol Exp Neurol 1999 October;58(10):1029-40). Stromal or moderate to strong periductal Tenascin-C expression in DCIS (ductal carcinoma in situ) correlates with tumor cell invasion. (Jahkola et al. Eur J Cancer 1998 October;34(11):1687-92. Tenascin-C expression at the invasion border of early breast cancer is a useful predictor of local and distant recurrence. Jahkola T, et al. Br J Cancer. 1998 December;78(11):1507-13). Tenascin (TN) is an extracellular matrix protein found in areas of cell migration during development and expressed at high levels in migratory glioma cells. Treasurywala S, Berens M E Glia 1998 October;24(2):236-43 Migration arrest in glioma cells is dependent on the alphaV integrin subunit. Phillips G R, Krushel L A, Crossin K L J Cell Sci 1998 April; 111 (Pt 8): 1095-104 Domains of tenascin involved in glioma migration. Finally, tenascin expression in hormone-dependent tissues of breast and endometrium indicate that Tenascin expression reflects malignant progression and is down-regulated by antiprogestins during terminal differentiation of rat mammary tumors (Vollmer et al. Cancer Res 1992 Sep. 1;52(17):4642-8)

Potential role of FCTR3 in oncologic disease progression:

Based on the bioactivity described in the medical literature for related molecules, FCTR3 may play a role in one or more aspects of tumor cell biology that alter the interactions of tumor epithelial cells with stromal components. In consideration, FCTR3 may play a role in the following malignant properties:

Autocrine/paracrine stimulation of tumor cell proliferation

Autocrine/paracrine stimulation of tumor cell survival and tumor cell resistance to cytotoxic therapy

Local tissue remodeling, paranechmal and basement membrane invasion and motility of tumor cells thereby contributing to metastasis.

Tumor-mediated immunosuppression of T-cell mediated immune effector cells and pathways resulting in tumor escape from immune surveilance.

Therapeutic intervention targeting FCTR3 in oncologic and central nervous system indications:

Predicted disease indications from expression profiling in 41 normal human tissues and 55 human cancer cell lines (see Example 2) include a subset of human gliomas, astrocytomas, mixed glioma/astrocytomas, renal cells carcinoma, breast adenocarcinoma, ovarian cancer, melanomas. Targeting of FCTR3 by human or humanized monoclonal antibodies designed to disrupt predicted interactions of FCTR3 with its cognate ligand may result in significant anti-tumor/anti-metastatic activity and the amelioration of associated symptomatology. Identification of small molecules that specifically/selectively interfere with downstream signaling components engaged by FCTR3/ligandinteractions would also be expected to result in significant anti-tumor/anti-metastatic activity and the amelioration of associated symptomatology. Likewise, modified antisense ribonucleotides or antisense gene expression constructs (plasmids, adenovirus, adeno-associated viruses, “naked” DNA approaches) designed to diminish the expression of FCTR3 transcripts/messenger RNA (mRNA) would be anticipated based on predicted properties of FCTR3 to have anti-tumor impact.

Based on the relatedness to neurestin and heregulins, as well as its high level expression in brain tissue, FCTR3 may also be used for remyelination in order to promote regeneration/repair/remyleination of injured central nervous system cells resulting from ischemia, brain trauma and various neurodegenerative diseases.. This postulate is based on reports indicating that neuregulin, glial growth factor 2, diminishes autoimmune demyelination and enhances remyelination in a chronic relapsing model for multiple sclerosis (Cannella et al. . Proc. Nat. Acad. Sci. 95: 10100-10105, 1998). The expression of the related molecule neurestin can be induced in external tufted cells during regeneration of olfactory sensory neurons.

FCTR4

FCTR4 is a plasma membrane protein related to NF-Kappa-B P65delta3 protein. The clone is expressed in fetal liver tissues.

The novel FCTR4 nucleic acid of 609 nucleotides (also referred to as 29692275.0.1) is shown in Table 4A. An ORF begins with an ATG initiation codon at nucleotides 99-101 and ends with a TAA codon at nucleotides 522-524. A putative untranslated region upstream from the initiation codon and downstream from the termination codon is underlined in Table 4A, and the start and stop codons are in bold letters.

TABLE 4A
FCTR4 Nucleotide Sequence

| | |
|---|---|
| CTGACATACTATATTAGTTGTTTGTTCACTGTCTCC | (SEQ ID NO:14) |
| ACTCCAGCTAGAATATAAGTTCCATACGGCAGAGTT | |
| TTTGTTCACTGCTATATTTTATAAGCATGAATGAAT | |
| GCATGAACGAATGGACTGATAACCCACAAGCCAAAG | |
| ACCTCCATGACCTGCCACTGCCCTCCTTTCATTTTA | |
| TTCTCACCTCTACCAATACTAAATCACCTAGTTATG | |
| TAAATACGATATGCACTTTCATGGCCCCTTGCTTTG | |
| TCATATGCTGTTCCCTTTGCCTGGAATATAAACTCT | |
| CAAAATACCATCCACATTTTAAAATCTTCTCCAGAA | |
| AGCTTCCTCTGTCCACCCCCACCCTCCCACCCCCAT | |
| ATAGAGTAAGTCAGTCTTTCCTTTGTGCTACATTTG | |
| TACCTGTATCTACAGTGGCTCTAATCAAACTGCACT | |
| GTGTCTCTCACTTCCTAGATTGTGAACTCTTTGAGG | |
| CTGAAGACTACTTATTCATCTCTTTACCTCCAATGC | |
| CTAGGACAGGACCTTCATAAAGCAACTACTCTATAA | |
| ATGTTGAAACATATGCATGACTATTCTGTAACAGGA | |
| ATGAAAATATGGCATTTCAAGAAGTCACTACTC | |

The FCTR4 protein encoded by SEQ ID NO:14 has 141 amino acid residues and is presented using the one-letter code in Table 4B. The Psort profile for FCTR4 predicts that this sequence has no N-terminal signal peptide and is likely to be localized at the plasma membrane with a certainty of 0.6000. The most likely cleavage site for a peptide is between amino acids 39 and 40, i.e., at the dash in the amino acid sequence ACT-CCA, based on the SignalP result. The predicted molecular weight of this protein is 16051.5 Daltons.

TABLE 4B
Encoded FCTR4 protein sequence

| | |
|---|---|
| MNECMNEWTDNPQAKDLHDLPLPSFHFILTSTNTKS | (SEQ ID NO:15) |
| PSYVNTICTFMAPCFVICCSLCLEYKLSKYHPHFKI | |
| FSRKLPLSTPTLPPPYRVSQSFLCATFVPVSTVALI | |
| KLHCVSHFLDCELFEAEDYLFISLPPMPRTGPS. | |

The predicted amino acid sequence was searched in the publicly available GenBank database FCTR4 protein showed 30 % identities (22 over 72 amino acids) and 43% homologies (31 over 72 amino acids) with hypothetical 10 kD protein of Trypanosoma cruzi (86 aa; ACC:Q99233) shown in Table 4C. The best homologies with a human protein were 54% identities (114 over 343 amino acids) with NF-Kappa-B P65delta3 protein (71 aa fragment; ACC:Q13313) (SEQ ID NO:77).

TABLE 4C
BLASTP of FCTR4 against protein sequences

BLAST X search results are shown below:

| | |
|---|---|
| ptnr:SPTREMBL-ACC:Q99233 HYPOTHETICAL 10 KD PROTEIN +3, 68, 0.60, 1, | (SEQ ID NO:73) |
| ptnr:SPTREMBL-ACC:Q16896 GABA RECEPTOR SUBUNIT - AEDES +3, 66, 0.81, 4 | (SEQ ID NO:74) |
| ptnr:SPTREMBL-ACC:O76473 GABA RECEPTOR SUBUNIT - LEPTI . . . +3, 66, 0.99, 2 | (SEQ ID NO:75) |
| ptnr:TREMBLNEW-ACC:AAD28317 F13J11.13 PROTEIN - Arabid . . . +3, 62, 0.99, 1 | (SEQ ID NO:76) |

Based upon homology, FCTR4 proteins and each homologous protein or peptide may share at least some activity.

FCTR5

FCTR5 is a protein bearing sequence homology to human complement CIR component precursor. The clone is expressed in breast, heart, lung, fetal lung, salivary gland, adrenal gland, spleen, kidney, and fetal kidney.

The novel FCTR5 nucleic acid of 1667 nucleotides (also referred to as 32125243.0.21) is shown in Table 5A. An ORF begins with an ATG initiation codon at nucleotides 34-36 and ends with a TGA codon at nucleotides 1495-1497. A putative untranslated region upstream from the initiation codon and downstream from the termination codon is underlined in Table 5A, and the start and stop codons are in bold letters.

TABLE 5A
FCTR5a Nucleotide Sequence (SEQ ID NO:16)

GTTCTCTCGCAGGTCCCAGATGTCCAGTTCCAGATGCCTGGACCCAGAGTGTGGGGGAAATATCTCTGGAGAAGCCCTCA
CTCCAAAGGCTGTCCAGGCGCAATGTGGTGGCTGCTTCTCTGGGGAGTCCTCCAGGCTTGCCCAACCCGGGGCTCCGTCC
TCTTGGCCCAAGAGCTACCCCAGCAGCTGACATCCCCCGGGTACCCAGAGCCGTATGGCAAAGGCCAAGAGAGCAGCACG
GACATCAAGGCTCCAGAGGGCTTTGCTGTGAGGCTCGTCTTCCAGGACTTCGACCTGGAGCCGTCCCAGGACTGTGCAGG
GGACTCTGTCACAATCTCATTCGTCGGTTCGGATCCAAGCCAGTTCTGTGGTCAGCAAGGCTCCCCTCTGGGCAGGCCCC
CTGGTCAGAGGGAGTTTGTATCCTCAGGGAGGAGTTTGCGGCTGACCTTCCGCACACAGCCTTCCTCGGAGAACAAGACT
GCCCACCTCCACAAGGGCTTCCTGGCCCTCTACCAAACCGTGGCTGTGAACTATAGTCAGCCCATCAGCGAGGCCAGCAG
GGGCTCTGAGGCCATCAACGCACCTGGAGACAACCCTGCCAAGGTCCAGAACCACTGCCAGGAGCCCTATTATCAGGCCG
CGGCAGCAGGGGCACTCACCTGTGCAACCCCAGGGACCTGGAAAGACAGACAGGATGGGGAGGAGGTTCTTCAGTGTATG
CCTGTCTGCGGACGACCAGTCACCCCCATTGCCCAGAATCAGACGACCCTCGGTTCTTCCAGAGCCAAGCTGGGCAACTT
CCCCTGGCAAGCCTTCACCAGTATCCACGGCCGTGGGGGCGGGGCCCTGCTGGGGGACAGATGGATCCTCACTGCTGCCC
ACACCATCTACCCCAAGGACAGTGTTTCTCTCAGGAAGAACCAGAGTGTGAATGTGTTCTTGGGCCACACAGCCATAGAT
GAGATGCTGAAACTGGGGAACCACCCTGTCCACCGTGTCGTTGTGCACCCCGACTACCGTCAGAATGAGTCCCATAACTT
TAGCGGGGACATCGCCCTCCTGGAGCTGCAGCACAGCATCCCCCTGGGCCCCAACGTCCTCCCGGTCTGTCTGCCCGATA
ATGAGACCCTCTACCGCAGCGGCTTGTTGGGCTACGTCAGTGGGTTTGGCATGGAGATGGGCTGGCTAACTACTGAGCTG
AAGTACTCGAGGCTGCCTGTAGCTCCCAGGGAGGCCTGCAACGCCTGGCTCCAAAAGAGACAGAGACCCGAGGTGTTTTC
TGACAATATGTTCTGTGTTGGGGATGAGACGCAAAGGCACAGTGTCTGCCAGGGGGACAGTGGCAGCCTCTATGTGGTAT
GGGACAATCATGCCCATCACTGGGTGGCCACGGGCATTGTGTCCTGGGGCATAGGGTGTGGCGAAGGGTATGACTTCTAC
ACCAAGGTGCTCAGCTATGTGGACTGGATCAAGGGAGTGATGAATGGCAAGAATTGACCCTGGGGGCTTGAACAGGGACT
GACCAGCACAGTGGAGGCCCCAGGCAACAGAGGGCCTGGAGTGAGGACTGAACACTGGGGTAGGGGGTGGGGGTTTCTCT
TGCAGTGGCTTGGTGCAACAGTGATGTGAATAGGATTTCCCTTTTTTTTTTTTTTTTTAAAAAAAAA

The FCTR5 protein encoded by SEQ ID NO: 16 has 487 amino acid residues, and is presented using the one-letter code in Table 5B. FCTR5 was searched against other databases using SignalPep and PSort search protocols. The FCTR5 protein is most likely microbody (peroxisome) (Certainty=0.6406) and seems to have no N-terminal signal sequence. The predicted molecular weight of FCTR5 protein is 53511.9 daltons.

TABLE 5B
Encoded FCTR5a protein sequence (SEQ ID NO:17).

MPGPRVWGKYLWRSPHSKGCPGAMWWLLLWGVLQACPTRGSVLLAQELPQQLTSPGYPEPYGKGQESSTDIKAPEGFAVRLVF
QDFDLEPSQDCAGDSVTISFVGSDPSQFCGQQGSPLGRPPGQREFVSSGRSLRLTFRTQPSSENKTAHLHKGFLALYQTVAVN
YSQPISEASRGSEAINAPGDNPAKVQNBCQEPYYQAAAAGALTCATPGTWKDRQDGEEVLQCMPVCGRPVTPIAQNQTTLGSS
RAKLGNFPWQAFTSIHGRGGGALLGDRWILTAAHTIYPKDSVSLRKNQSVNVFLGHTAIDEMLKLGNHPVHRVVVHPDYRQNE
SHNFSGDIALLELQHSIPLGPNVLPVCLPDNETLYRSGLLGYVSGFGMEMGWLTTELKYSRLPVAPREACNAWLQKRQRPEVF
SDNMFCVGDETQRHSVCQGDSGSLYVVWDNHAHHWVATGIVSWGIGCGEGYDFYTKVLSYVDWIKGVMNGKN

An alternative embodiment, FCTR5b, is a 1691 base sequence shown in Table 5C.

TABLE 5C
FCTR5b Nucleotide Sequence (SEQ ID NO:18)

TTTTTTTTTAAAAAAAAAAAAAAAAAGGGAAATCCTATTCACATCACTGTTGCACCAAGCCACTGCAAGAGAAACCCCCACCC
CCTACCCCAGTGTTCAGTCCTCACTCCAGGCCCTCTGTTGCCTGGGGCCTCCACTGTGCTGGTCAGTCCCTGTTCAAGCCCCC
AGGGTCAATTCTTGCCATTCATCACTCCCTTGATCCAGTCCACATAGCTGAGCACCTTGGTGTAGAAGTCATACCCTTCGCCA
CACCCTATGCCCCAGGACACAATGCCCGTGGCCACCCAGTGATGGGCATGATTGTCCCATACCACATAGAGGCTGCCACTGTC
CCCCTGGCAGACACTGTGCCTTTGCGTCTCATCCCCAACACAGAACATATTGTCAGAAAACACCTCGGGTCTCTGTCTCTTTT
GGAGCCAGGCGTTGCAGGCCTCCCTGGGAGCTACAGGCAGCCTCGAGTACTTCAGCTCAGTAGTTAGCCAGCCCATCTCCATG
CCAAACCCACTGACGTAGCCCAACAAGCCGCTGCGGTAGAGGGTCTCATTATCGGGCAGACAGACCGGGAGGACGTTGGGGCC
CAGGGGGATGCTGTGCTGCAGCTCCAGGAGGGCGATGTCCCCGCTAAAGTTATGGGACTCATTCTGACGGTAGTCGGGGTGCA
CAACGACACGGTGGACAGGGTGGTTCCCCAGTTTCAGCATCTCATCTATGGCTGTGTGGCCCAAGAACACATTCACACTCTGG
TTCTTCCTGAGAGAAACACTGTCCTTGGGGTAGATGGTGTGGGCAGCAGTGAGGATCCATCTGTCCCCCAGCAGGGCCCCGCC
CCCACGGCCGTGGATACTGGTGAAGGCTTGCCAGGGGAAGTTGCCCAGCTTGGCTCTGGAAGAACCGAGGGTCGTCTGATTCT
GGGCAATGGGGGTGACTGGCCGTCCGCAGACAGGCATACACTGAAGAACCTCCTCCCCATCCTGTCTGTCTTTCCAGGTCCCT
GGGGTTGCACAGGTGAGTGCCCCTGCTGCCGCGGCCTGATAATAGGGCTCCTGGCAGTGGTTCTGGACCTTGGCAGGGTTGTC
TCCAGGTGCGTTGATGGCCTCAGAGCCCCTGCTGGCCTCGCTGATGGGCTGACTATAGTTCACAGCCACGGTTTGGTAGAGGG
CCAGGAAGCCCTTGTGGAGGTGGGCAGTCTTGTTCTCCGAGGAAGGCTGTGTGCGGAAGGTCAGCCGCAAACTCCTCCCTGAG
GATACAAACTCCCTCTGACCAGGGGGCCTGCCCAGAGGGGAGCCTTGCTGACCACAGAACTGGCTTGGATCCGAACCGACGAA
TGAGATTGTGACAGAGTCCCCTGCACAGTCCTGGGACGGCTCCAGGTCGAAGTCCTGGAAGACGAGCCTCACAGCAAAGCCCT
CTGGAGCCTTGATGTCCGTGCTGCTCTCTTGGCCTTTGCCATACGGCTCTGGGTACCCGGGGGATGTCAGCTGCTGGGGTAGC
TCTTGGGCCAAGAGGACGGAGCCCCGGGTTGGGCAAGCCTGGAGGACTCCCCAGAGAAGCAGCCACCACATTGCGCCTGGACA
GCCTTTGGAGTGAGGGCTTCTCCAGAGATATTTCCCCCACACTCTGGGTCCAGGCATCTGGAACTGGACATCTGGGACCTGCG
AGAGAACTGGCCCAGGATAGGGAACAAAAGG

The FCTR5b protein encoded by SEQ ID NO: 18 has 487 amino acid residues, and is presented using the one-letter code in Table 5D. FCTR5 was searched against other databases using SignalPep and PSort search protocols. The FCTR5b protein is most likely microbody (peroxisome) (Certainty=0.6406) and seems to have no N-terminal signal sequence. The predicted molecular weight of FCTR5 protein is 53511.9 daltons.

TABLE 5D
Encoded FCTR5b protein sequence (SEQ ID NO:19).

MPGPRVWGKYLWRSPHSKGCPGAMWWLLLWGVLQACPTRGSVLLAQQLPQQLTSPGYPEPYGKGQESSTDIKAPEGFAVRLVF
QDFDLEPSQDCAGDSVTISFVGSDPSQFCGQQGSPLGRPPGQREFVSSGRSLRLTFRTQPSSENKTAHLHKGFLALYQTVAVN
YSQPISEASRGSEAINAPGDNPAKVQNHCQEPYYQAAAAGALTCATPGTWKDRQDGEEVLQCMPVCGRPVTPIAQNQTTLGSS
RAKLGNFPWQAFTSIHGRGGGALLGDRWILTAAHTIYPKDSVSLRKNQSVNVFLGHTAIDEMLKLGNHPVHRVVVHPDYRQNE
SHNFSGDIALLELQHSIPLGPNVLPVCLPDNETLYRSGLLGYVSGFGMEMGWLTTELKYSRLPVAPREACNAWLQKRQRPEVF
SDNMFCVGDETQRHSVCQGDSGSLYVVWDNHAHHWVATGIVSWGIGCGEGYDFYTKVLSYVDWIKGVMNGKN

The predicted amino acid sequence was searched in the publicly available GenBank database FCTR5a protein showed 58 % identities (177 over 302 amino acids) and 74% homologies (226 over 302 amino acids) with human complement CIR component precursor (EC 3.4.21.41) (705 aa.; ACC:P00736). Based upon homology, FCTR5 proteins and each homologous protein or peptide may share at least some activity.

In a search of sequence databases, it was found, for example, that the nucleic acid sequence the nucleotides 17-1594 of FCTR5a have 1575 of 1578 bases (99 %) identical to Homo sapiens complement C1r-like proteinase precursor (GENBANK-ID: XM_{—}007061.1) (SEQ ID NO:78) (Table 5E).

TABLE 5E
BLASTN of FCTR5a against Homo sapiens complement C1r-like
proteinase precursor (SEQ ID NO:78)

>GI|11436767|REF|XM_007061 .1| HOMO SAPIENS
COMPLEMENT C1R-LIKE PROTEINASE PRECURSOR, (LOC51279), MRNA
LENGTH = 3318
SCORE = 3104 BITS (1566), EXPECT = 0.0
IDENTITIES = 1575/1578 (99%)
STRAND = PLUS/PLUS

| | | |
|---|---|---|
| QUERY: 17 | CAGATGTCCAGTTCCAGATGCCTGGACCCAGAGTGTGGGGGAAATATCTCTGGAGAAGCC | 76 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: 1 | CAGATGTCCAGTTCCAGATGCCTGGACCCAGAGTGTGGGGGAAATATCTCTGGAGAAGCC | 60 |
| QUERY: 77 | CTCACTCCAAAGGCTGTCCAGGCGCAATGTGGTGGCTGCTTCTCTGGGGAGTCCTCCAGG | 136 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: 61 | CTCACTCCAAAGGCTGTCCAGGCGCAATGTGGTGGCTGCTTCTCTGGGGAGTCCTCCAGG | 120 |
| QUERY: 137 | CTTGCCCAACCCGGGGCTCCGTCCTCTTGGCCCAAGAGCTACCCCAGCAGCTGACATCCC | 196 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: 121 | CTTGCCCAACCCGGGGCTCCGTCCTCTTGGCCCAAGAGCTACCCCAGCAGCTGACATCCC | 180 |
| QUERY: 197 | CCGGGTACCCAGAGCCGTATGGCAAAGGCCAAGAGAGCAGCACGGACATCAAGGCTCCAG | 256 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: 181 | CCGGGTACCCAGAGCCGTATGGCAAAGGCCAAGAGAGCAGCACGGACATCAAGGCTCCAG | 240 |
| QUERY: 257 | AGGGCTTTGCTGTGAGGCTCGTCTTCCAGGACTTCGACCTGGAGCCGTCCCAGGACTGTG | 316 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: 241 | AGGGCTTTGCTGTGAGGCTCGTCTTCCAGGACTTCGACCTGGAGCCGTCCCAGGACTGTG | 300 |
| QUERY: 317 | CAGGGGACTCTGTCACAATCTCATTCGTCGGTTCGGATCCAAGCCAGTTCTGTGGTCAGC | 376 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: 301 | CAGGGGACTCTGTCACAATCTCATTCGTCGGTTCGGATCCAAGCCAGTTCTGTGGTCAGC | 360 |
| QUERY: 377 | AAGGCTCCCCTCTGGGCAGGCCCCCTGGTCAGAGGGAGTTTGTATCCTCAGQGAGGAGTT | 436 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: 361 | AAGGCTCCCCTCTGGGCAGGCCCCCTGGTCAGAGGGAGTTTGTATCCTCAGGGAGGAGTT | 420 |
| QUERY: 437 | TGCGGCTGACCTTCCGCACACAGCCTTCCTCGGAGAACAAGACTGCCCACCTCCACAAGG | 496 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: 421 | TGCGGCTGACCTTCCGCACACAGCCTTCCTCGGAGAACAAGACTGCCCACCTCCACAAGG | 480 |
| QUERY: 497 | GCTTCCTGGCCCTCTACCAAACCGTGGCTGTGAACTATAGTCAGCCCATCAGCGAGGCCA | 556 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: 481 | GCTTCCTGGCCCTCTACCAAACCGTGGCTGTGAACTATAGTCAGCCCATCAGCGAGGCCA | 540 |
| QUERY: 557 | GCAGGGGCTCTGAGGCCATCAACGCACCTGGAGACAACCCTGCCAAGGTCCAGAACCACT | 616 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: 541 | GCAGGGGCTCTGAGGCCATCAACGCACCTGGAGACAACCCTGCCAAGGTCCAGAACCACT | 600 |
| QUERY: 617 | GCCAGGAGCCCTATTATCAGGCCGCGGCAGCAGGGGCACTCACCTGTGCAACCCCAGGGA | 676 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: 601 | GCCAGGAGCCCTATTATCAGGCCGCGGCAGCAGGGGCACTCACCTGTGCAACCCCAGGGA | 660 |
| QUERY: 677 | CCTGGAAAGACAGACAGGATGGGGAGGAGGTTCTTCAGTGTATGCCTGTCTCCGGACGGC | 736 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: 661 | CCTGGAAAGACAGACAGGATGGGGAGGAGGTTCTTCAGTGTATGCCTGTCTGCGGACGGC | 720 |
| QUERY: 737 | CAGTCACCCCCATTGCCCAGAATCAGACGACCCTCGGTTCTTCCAGAGCCAAGCTGGGCA | 796 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: 721 | CAGTCACCCCCATTGCCCAGAATCAGACGACCCTCGGTTCTTCCAGAGCCAAGCTGGGCA | 780 |
| QUERY: 797 | ACTTCCCCTGGCAAGCCTTCACCAGTATCCACGGCCGTGGGGOCGGGGCCCTGCTGGGGG | 856 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: 781 | ACTTCCCCTGGCAAGCCTTCACCAGTATCCACGGCCGTGGGGGCGGGGCCCTGCTGGGGG | 840 |
| QUERY: 857 | ACAGATGGATCCTCACTGCTGCCCACACCATCTACCCCAAGGACAGTGTTTCTCTCAGGA | 916 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: 841 | ACAGATGGATCCTCACTGCTGCCCACACCGTCTACCCCAAGGACAGTGTTTCTCTCAGGA | 900 |
| QUERY: 917 | AGAACCAGAGTGTGAATGTGTTCTTGGGCCACACAGCCATAGATGAGATGCTGAAACTGG | 976 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: 901 | AGAACCAGAGTGTGAATGTGTTCTTGGGCCACACAGCCATAGATGAGATGCTGAAACTGG | 960 |
| QUERY: 977 | GGAACCACCCTGTCCACCGTGTCGTTGTGCACCCCGACTACCGTCAGAATGAGTCCCATA | 1036 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: 961 | GGAACCACCCTGTCCACCGTGTCGTTGTGCACCCCGACTACCGTCAGAATGAGTCCCATA | 1020 |
| QUERY: 1037 | ACTTTAGCGGGGACATCGCCCTCCTGGAGCTGCAGCACAGCATCCCCCTGGGCCCCAACG | 1096 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: 1021 | ACTTTAGCGGGGACATCGCCCTCCTGGAGCTGCAGCACAGCATCCCCCTGGGCCCCAACG | 1080 |
| QUERY: 1097 | TCCTCCCGGTCTGTCTGCCCGATAATGAGACCCTCTACCGCAGCGGCTTGTTGGGCTACG | 1156 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: 1081 | TCCTCCCGGTCTGTCTGCCCGATAATGAGACCCTCTACCGCAGCGGCTTGTTGGGCTACG | 1140 |
| QUERY: 1157 | TCAGTGGGTTTGGCATGGAGATGGGCTGGCTAACTACTGAGCTGAAGTACTCGAGGCTGC | 1216 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: 1141 | TCAGTGGGTTTGGCATGGAGATGGGCTGGCTAACTACTGAGCTGAAGTACTCGAGGCTGC | 1200 |
| QUERY: 1217 | CTGTAGCTCCCAGGGAGGCCTGCAACGCCTGGCTCCAAAAGAGACAGAGACCCGAGGTGT | 1276 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: 1201 | CTGTAGCTCCCAGGGAGGCCTGCAACGCCTGGCTCCAAAAGAGACAGAGACCCGAGGTGT | 1260 |
| QUERY: 1277 | TTTCTGACAATATGTTCTGTGTTGGGGATGAGACGCAAAGGCACAGTGTCTGCCAGGGGG | 1336 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: 1261 | TTTCTGACAATATGTTCTGTGTTGGGGATGAGACGCAAAGGCACAGTGTCTGCCAGGGGG | 1320 |
| QUERY: 1337 | ACAGTGGCAGCCTCTATGTGGTATGGGACAATCATGCCCATCACTGGGTGGCCACGGGCA | 1396 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: 1321 | ACAGTGGCAGCGTCTATGTGGTATGGGACAATCATGCCCATCACTGGGTGGCCACGGGCA | 1380 |
| QUERY: 1397 | TTGTGTCCTGGGGCATAGGGTGTGGCGAAGGGTATGACTTCTACACCAAGGTGCTCAGCT | 1456 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: 1381 | TTGTGTCCTGGGGCATAGGGTGTGGCGAAGGGTATGACTTCTACACCAAGGTGCTCAGCT | 1440 |
| QUERY: 1457 | ATGTGGACTGGATCAAGGGAGTGATGAATGGCAAGAATTGACCCTGGGGGCTTGAACAGG | 1516 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: 1441 | ATGTGGACTGGATCAAGGGAGTGATGAATGGCAAGAATTGACCCTGGGGGCTTGAACAGG | 1500 |
| QUERY: 1517 | GACTGACCAGCACAGTGGACGCCCCAGGCAACAGAGGGCCTGGAGTGAGGACTGAACACT | 1576 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: 1501 | GACTGACCAGCACAGTGGAGGCCCCAGGCAACAGAGGGCCTGGAGTGAGGACTGAACACT | 1550 |
| QUERY: 1577 | GGGGTAGGGGGTGGGGGT | 1594 |
| | \|\|\|\|\|\|\|\|\|\|\|\|\|\|\|\|\|\| | |
| SBJCT: 1551 | GGGGTAGGGGTTGGGGGT | 1578 |

In this search it was also found that the FCTR5a nucleic acid had homology to three fragments of Homo sapiens complement component 1, r subcomponent. It has 102 of 117 bases (87%) identical to 1458-1574, 82 of 94 bases (87%) identical to 2052-2145, and 54 of 63 bases (85%) identical to 1678-1740 all fragments of Homo sapiens complement component 1, r subcomponent (GenBank Acc: NM_{—}001733.1) (Table 5F).

TABLE 5F
BLASTN of FCTR5a against Homo sapiens complement
component 1, r subcomponent (SEQ ID NO:79)

>GI|4502492|REF|NM_001733.1| HOMO SAPIENS
COMPLEMENT COMPONENT 1, R SUBCOMPONENT (C1R), MRNA
LENGTH = 2386
SCORE = 113 BITS (57), EXPECT = 3E-22
IDENTITIES = 102/117 (87%)
STRAND = PLUS/PLUS

| | | |
|---|---|---|
| QUERY: 783 | AGCCAAGCTGGGCAACTTCCCCTGGCAAGCCTTCACCAGTATCCACGGCCGTGGGGGCGG | 842 |
| | \|\|\|\|\|\|\| \|\|\|\|\|\|\|\|\|\|\|\|\|\|\|\|\|\|\| \| \|\|\|\|\|\|\| \|\|\|\|\|\|\|\| \|\| \|\|\|\|\|\|\|\| | |
| SBJCT: 1458 | AGCCAAGATGGGCAACTTCCCCTGGCAGGTGTTCACCAACATCCACGGGCGCGGGGGCGG | 1517 |
| QUERY: 843 | GGCCCTGCTGGGGGACAGATGGATCCTCACTGCTGCCCACACCATCTACCCCAAGGA | 899 |
| | \|\|\|\|\|\|\|\|\|\|\|\| \|\|\| \| \|\|\|\|\|\|\|\|\|\|\| \|\|\|\|\|\|\|\|\|\|\|\| \| \|\| \|\|\|\|\|\|\|\| | |
| SBJCT: 1518 | GGCCCTGCTGGGCGACCGCTGGATCCTCACAGCTGCCCACACCCTGTATCCCAAGGA | 1574 |

SCORE = 91.7 BITS (46), EXPECT = 1E-15
IDENTITIES = 82/94 (87%)
STRAND = PLUS/PLUS

| | | |
|---|---|---|
| QUERY: 1380 | CTGGGTGGCCACGGGCATTGTGTCCTGGGGCATAGGGTGTGGCGAAGGGTATGACTTCTA | 1439 |
| | \|\|\|\|\|\|\|\|\|\|\|\|\|\|\|\|\|\| \|\|\|\|\|\|\|\|\|\|\|\|\|\| \|\|\|\|\| \|\| \|\| \|\|\|\| \|\|\|\|\|\| | |
| SBJCT: 2052 | CTGGGTGGCCACGGGCATCGTGTCCTGGGGCATCGGGTGCAGCAGGGGCTATGGCTTCTA | 2111 |
| QUERY: 1440 | CACCAAGGTGCTCAGCTATGTGGACTGGATCAAG | 1473 |
| | \|\|\|\|\|\| \|\|\|\|\|\|\| \|\|\| \|\|\|\|\|\|\|\|\|\|\|\|\|\|\| | |
| SBJCT: 2112 | CACCAAAGTGCTCAACTACGTGGACTGGATCAAG | 2145 |

SCORE = 54.0 BITS (27), EXPECT = 2E-04
IDENTITIES = 54/63 (85%)
STRAND = PLUS/PLUS

| | | |
|---|---|---|
| QUERY: 1006 | CACCCCGACTACCGTCAGAATGAGTCCCATAACTTTAGCGGGGACATCGCCCTCCTGGAG | 1065 |
| | \|\|\|\|\| \|\|\|\|\|\|\|\|\|\|\|\| \|\|\|\|\|\|\|\| \| \|\| \|\|\| \|\|\|\|\|\|\|\|\|\|\|\|\|\| \|\|\|\|\|\| | |
| SBJCT: 1678 | CACCCGGACTACCGTCAGGATGAGTCCTACAATTTTGAGGGGGACATCGCCCTGCTGGAG | 1737 |
| QUERY: 1066 | CTG | 1068 |
| | \|\|\| | |
| SBJCT: 1738 | CTG | 1740 |

The amino acid sequence of the protein of FCTR5a 485 of 487 amino acid residues (99%) identical to, and 487 of 487 residues (100%) positive with, the 487 amino acid complement C1r-like proteinase precursor from Homo sapiens (GenBank-ACC: AAF44349.1) (SEQ ID NO:80) (Table 5G).

TABLE 5G
BLASTP of FCTR5a and b against Complement C1R-like
proteinase precursor (SEQ ID NO:80)

```
>GI|7706083|REF|NP_057630.1| COMPLEMENT
C1R-LIKE PROTEINASE PRECURSOR, [HOMO SAPIENS]
GI|11436768|REF|XP_007061.1| COMPLEMENT
C1R-LIKE PROTEINASE PRECURSOR, [HOMO SAPIENS]
GI|7271475|GB|AAF44349.1|AF178985_1 (AF178985) COMPLEMENT
C1R-LIKE PROTEINASE PRECURSOR [HOMO SAPIENS]
                    LENGTH = 487
SCORE = 972 BITS (2513), EXPECT = 0.0
IDENTITIES = 485/487 (99%), POSITIVES = 487/487 (100%)
                                                                                                                  R
```

| | | |
|---|---|---|
| QUERY: 1 | MPGPRVWGKYLWRSPHSKGCPGAMWWLLLWGVLQACPTRGSVLLAQELPQQLTSPGYPEP | 60 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: 1 | MPGPRVWGKYLWRSPHSKGCPGAMWWLLLWGVLQACPTRGSVLLAQELPQQLTSPGYPEP | 60 |
| QUERY: 61 | YGKGQESSTDIKAPEGFAVRLVFQDFDLEPSQDCAGDSVTISFVGSDPSQFCGQQGSPLG | 120 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: 61 | YGKGQESSTDIKAPEGFAVRLVFQDFDLEPSQDCAGDSVTISFVGSDPSQFCGQQGSPLG | 120 |
| QUERY: 121 | RPPGQREFVSSGRSLRLTFRTQPSSENKTAHLHKGFLALYQTVAVNYSQPISEASRGSEA | 180 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: 121 | RPPGQREFVSSGRSLRLTFRTQPSSENKTAHLHKGFLALYQTVAVNYSQPISEASRGSEA | 180 |
| QUERY: 181 | INAPGDNPAKVQNHCQEPYYQAAAAGALTCATPGTWKDRQDGEEVLQCMPVCGRPVTPIA | 240 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: 181 | INAPGDNPAKVQNECQEPYYQAAAAGALTCATPGTWKDRQDGEEVLQCMPVCGRPVTPIA | 240 |
| QUERY: 241 | QNQTTLGSSRAXLGNFPWQAFTSIHGRGGGALLGDRWILTAAHTIYPKDSVSLRKNQSVN | 300 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|+\|\|\|\|\|\|\|\|\|\|\|\|\|\|\| | |
| SBJCT: 241 | QNQTTLGSSRAKLGNFPWQAFTSIEGRGGGALLGDRWILTAAHTVYPKDSVSLRKNQSVN | 300 |
| QUERY: 301 | VFLGHTAIDEMLKLGNRPVERVVVHPDYRQNESENFSGDIALLELOHSIPLGPNVLPVCL | 360 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: 301 | VFLGHTAIDEMLKLGNHPVHRVVVHPDYRQNESHNFSGDIALLELQHSIPLGPNVLPVCL | 360 |
| QUERY: 361 | PDNETLYRSGLLGYVSGFGMEMGWLTTELKYSRLPVAPREACNAWLQKRQRPEVFSDNMF | 420 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: 361 | PDNETLYRSGLLGYVSGFGMEMGNLTTELKYSRLPVAPREACNAWLQKRQRPEVFSDNMF | 420 |
| QUERY: 421 | CV0DETQRHSVCQGDSGSLYVVWDNHAHHWVATGIVSWGIGCGEGYDFYTKVLSYVDWIK | 480 |
| | \|\|\|\|\|\|\|\|\|\|\|\|\|\|\|\|\|\|+\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: 421 | CVGDETQRBSVCQGDSGSVYVVWDNHAHRHVATGIVSWGIGCGEGYDFYTKVLSYVDWIE | 480 |
| QUERY: 481 | GVMNGKN | 487 |
| | \|\|\|\|\|\|\| | |
| SBJCT: 481 | GVMNGKN | 487 |

R = AT RESIDUE 46, FCTR5B DIFFERS FROM FCTRSA IN THAT Q46R. THE REST OF THE HOMOLOGY IS THE SAME.

The full amino acid sequence of the protein of FCTR5a has 175 of 303 amino acid residues (58%) identical to, and 226 of 303 residues (74%) positive with the 400-701 amino acid segment, 72 of 157 residues (45%) identical and 94 of 157 residues (59%/0) positive with amino acids 1-155, and 36 of 139 residues (25%) identical and 58 of 139 residues (40%) positive with amino acids 188-312 of the 705 amino acid Complement C1R Component Precursor from Homo sapiens (GenBank-ACC: AAAS1851.1) (SEQ ID NO:43) (Table 5H).

TABLE 5H
BLASTP of FCTR5a and b against Complement C1R
Component Precursor (SEQ ID NO:81)

>GI|115204|SP|P00736|C1R_HUMAN COMPLEMENT
C1R COMPONENT PRECURSOR
GI|67614|PIR|C1HURB COMPLEMENT SUBCOMPONENT
C1R (EC 3.4.21.41) PRECURSOR - HUMAN
GI|179644|GB|AAA51851.1| (M14058) HUMAN COMPLEMENT
C1R [HOMO SAPIENS]
LENGTH = 705
SCORE = 361 BITS (928), EXPECT = 8E-99
IDENTITIES = 175/303 (58%), POSITIVES = 226/303 (74%), GAPS = 9/303 (2%)

| | | |
|---|---|---|
| QUERY: 189 | AXVQNHCQEPYYQ--------AAAAGALTCATPGTWKDRQDGEEVLQCMPVCGRPVTPIA | 240 |
| | \|++\| +\| \|\|\|\|+ \| \|\| \| \|\|+ \| \|\|++ +\|+\|\|\|\|+\|\| \|+ | |
| SBJCT: 400 | ARIQYYCEEPYYKMQTRAGSRESEQGVYTCTAQGIWKNEQKGEKIPRCLPVCGKPVNPVE | 459 |
| QUERY: 241 | QNQTTLGSSRAKLGNFPWQAFTSIEGRGGGALLGDRWILTAAHTIYPKDSVSLRKNQSVN | 300 |
| | \| \| +\| +\|\|+\|\|\|\|\|\| \|\|+\|\|\|\|\|\|\|\|\|\|\|\|\|\|\|\|\|\|\|\|\|+\|\|\|+ + + \| \|++ | |
| SBJCT: 460 | QRQRIIGGQKAKMGNFPWQVFTNIEGRGGGALLGDRWILTAAETLYPKEEEA-QSNASLD | 518 |
| QUERY: 301 | VFLGHTAIDEMLKLGNHPVHRVVVHPDYRQNESHNFSGDIALLELQESIPLGPNVLPVCL | 360 |
| | \|\|\|\|\|\| ++\|++\|\|\|\|\|\|+ \|\| \|\|\|\|\|\|\|+\|\|+\|\| \|\|\|\|\|\|\|\|++\|+ \|\|\|\|+\|\|+\|\| | |
| SBJCT: 519 | VFLGHTNVEELMKLGNHPIRRVSVEPDYRQDESYNFEGDIALLELENSVTLGPNLLPICL | 578 |
| QUERY: 361 | PDNETLYRSGLLGYVSGFGMEMGWLTTELKYSRLPVAPREACNAWLQKRQRPEVFSDNMF | 420 |
| | \|\|\|+\| \| \|\|+\|\|\|\|\|\|\|+ + +\|++ \|\|\|\|\| +\|\| \|\|+ + \| +\|\|\| \|\|\| | |
| SBJCT: 579 | PDNDTFYDLGLMGYVSGRGVMEEKIAHDLRFVRLPVANPQACENWLRGKNRMDVFSQNMB | 638 |
| QUERY: 421 | CVGDETQRHSVCQGDSGSLYVVWDNHAHHWVATGIVSWGIGCGEGYDFYTKVLSYVDWIK | 480 |
| | \| \| + + \|\|\|\|\|\| ++ \| \| + \|\|\|\|\|\|\|\|\|\|\|\|\| \|\| \|\|\|\|\|\|+\|\|\|\|\|\| | |
| SBJCT: 639 | CAGHPSLKQDACQGDSGGVFAVRDPNTDRWVATGIVSWGIGCSRGYGFYTKVLNYVDWIK | 698 |
| QUERY: 481 | GVM | 483 |
| | \| | |
| SBJCT: 699 | REM | 701 |

SCORE = 122 BITS (306), EXPECT = 1E-26
IDENTITIES = 72/157 (45%), POSITIVES = 94/157 (59%), GAPS = 3/157 (1%)
R

| | | |
|---|---|---|
| QUERY: 24 | MWWLLLWGVLQACPTRGSVLLAQELPQQLTSPGYPEPYGKGQESSTDIKAPEGFAVRLVF | 83 |
| | \|\| \| \| \| \|\|+ + \|+\| ++\|\|\| +\|+\|\| \|++\| \| \| \|+ \|+\|\|\| | |
| SBJCT: 1 | MWLLYLLVPALFCRAGGSIPIPQKLFGEVTSPLFPKPYPNNFETTTVITVPTGYRVKLVF | 60 |
| QUERY: 84 | QDFDLEPSQDCAGDSVTISFVGSDPSQFCGQQGSPLGRPPGQREFVSSGRSLRLTFRTQP | 143 |
| | \| \|\|\|\|\|\|+ \| \| \| \|\| +\|\|\|\| \|\|\|\|\| \|\|\|++\|\|+\| \| + \|\|\| \| | |
| SBJCT: 61 | QQFDLEPSEGCFYDYVKISADKKSLGRFCGQLGSPLGNPPGKKEFMSQGNKMLLTFETDF | 120 |
| QUERY: 144 | SS-ENKTAHLEKGFLALYQTVAVNYSQPISEASRGSE | 179 |
| | \|+ \|\| \| +\|\|\|\|\| \|\| \|\|+ + \| + \| \| | |
| SBJCT: 121 | SNEENGTIMFYKGFLAYYQ--AVDLDECASRSKSGEE | 155 |

SCORE = 36.3 BITS (83), EXPECT = 0.93
IDENTITIES = 36/139 (25%), POSITIVES = 58/139 (40%), GAPS = 17/139 (12%)
R

| | | |
|---|---|---|
| QUERY: 35 | ACPTRGSVLLAQELPQQLTSPGYPEPYGKGQESSTDIKAPEGFAVRLVF-QDFDLEPSQD | 93 |
| | +\| \| \| \| ++\| \|\| \| + \|+ \| + \| \| + \|\|++ \| | |
| SBJCT: 188 | SCQAECSSELYTEASGYISSLEYPRSYPPDLRCNYSIRVERGLTLHLKFLEPFDIDDHQQ | 247 |
| QUERY: 94 | --CAGDSVTISFVGSDpSQFCGOQGSPLGRPPGQREFVSSGRSLRLTFRTQPSSENKTAH | 151 |
| | \| \| + \| \| + +\|\|\|+\| \|\|\| + +\| ++ \| \| \| \| ++ | |
| SBJCT: 248 | VECPYDQLQIYANGKNIGEFCGKQ-----RPP---DLDTSSNAVDLLFFTDESGDS---- | 295 |
| QUERY: 152 | LHKGFLALYQTVAVNYSQP | 170 |
| | +\|+ \| \| + \|\| | |
| SBJCT: 29G | --RGWKLRYTTEIIKCPQP | 312 |

R = AT RESIDUE 46, FCTR5B DIFFERS FROM FCTR5A IN THAT Q46R. THE REST OF THE HOMOLOGY IS THE SAME.

Based upon homology, FCTR5 proteins and each homologous protein or peptide may share at least some activity.

FCTR6

The novel nucleic acid of 1078 nucleotides FCTR6a (also designated 27455183.0.19) encoding a novel human blood coagulation factor XI-like protein is shown in Table 6A. An ORF was identified beginning with an ATG initiation codon at nucleotides 243-245 and ending with a TAA codon at nucleotides 1044-1046. A putative untranslated region upstream from the initiation codon and downstream from the termination codon is underlined in Table 6A, and the start and stop codons are in bold letters.

TABLE 6A
FCTR6a Nucleotide Sequence (SEQ ID NO:20)

TTGATCCGTGCCAAGTGGCTTTTTGTGGGCTCTGTAGAGTGCTCTAAACCCAGCTCGGCCTTTGCTGTATTAGACAGAAGCAC
CTCATTCATATCCCTGGGGCCCCTGATGGTGCAGTGGTCTGGCTGTGGTCTGCACACCAGCTATTCTGTTTTGTTTTGTTTTG
TTTTTTTCCTACCTTTTTCCAATCCTCACACCTTCTGATCAACAGCCCCAGTAGGGTTTAAAGGTCCTAGAGCTACATGGGAT
TTAGGTTTCTGGGCACAGCCAATTCTGCCACTTTTGAGACTTCCCTTCCCCTTCCACTTGCCCCTCTCTGGTTCTCTGCCACC
AGTCCAGAAGAACTGAGTGTCGTGCTGGGGACCAACGACTTAACTAGCCCATCCATGGAAATAAAGGAGGTCGCCAGCATCAT
TCTTCACAAAGACTTTAAGAGAGCCAACATGGACAATGACATTGCCTTGCTGCTGCTGGCTTCGCCCATCAAGCTCGATGACC
TGAAGGTGCCCATCTGCCTCCCCACGCAGCCCGGCCCTGCCACATGGCGCGAATGCTGCGTGGCAGGTTGGGGCCAGACCAAT
GCTGCTGACAAAAACTCTGTGAAAACGGATCTGATGAAAGTGCCAATGGTCATCATGGACTGCGAGGAGTGTTCAAAGATGTT
TCCAAAACTTACCAAAAATATGCTGTGTGCCGGATACAAGAATGAGAGCTATGATGCCTGCAAGGGTGACAGTGGGGGGCCTC
TGGTCTGCACCCCAGAGCCTGGTGAGAAGTGGTACCAGGTGGGCATCATCAGCTGGGGAAAGAGCTGTGGAGATAAGAACACC
CCAGGGATATACACCTCGTTGGTGAACTACAACCTCTGGATCGAGAAAGTGACCCAGCTAGGAGGCAGGCCCTTCAATGCAGA
GAAAAGGAGGACTTCTGTCAAACAGAAACCTATGGGCTCCCCAGTCTCGGGAGTCCCAGAGCCAGGCAGCCCCAGATCCTGGC
TCCTGCTCTGTCCCCTGTCCCATGTGTTGTTCAGAGCTATTTTGTACTGATAATAAAATAGAGGCTATTCTTTCAACCGAAA

The FCTR6a protein encoded by SEQ ID NO:20 has 267 amino acid residues and is presented using the one-letter code in Table 6B. FCTR6a was searched against other databases using SignalPep and PSort search protocols. The PCTR6a protein is most likely mitochondrial matrix space (Certainty=0.4372) and seems to have no N-terminal signal sequence. The predicted molecular weight of FCTR6a protein is 29412.8 daltons.

TABLE 6B!Encoded FCTR6a protein sequence (SEQ ID NO:21).
MGFRFLGTANSATFETSLPLPLAPLWFSATSPEELSVVLGTNDLTSPSMEIKEVASIILHKDFKRANMDNDIALLLLASPIKL
DDLKVPICLPTQPGPATWRECWVAGWGQTNAADKNSVKTDLMKVPMVIMDWEECSKMFPKLTKNMLCAGYKNESYDACKGDSG
GPLVCTPEPGEKWYQVGIISWGKSCGDKNTPGIYTSLVNYNLWIEKVTQLGGRPENAEKRRTSVKQKPMGSPVSGVPEPGSPR
SWLLLCPLSHVLFRAILY

In an alternative embodiment, FCTR6b (alternatively referred to as 27455183.0.145) has the 1334 residue sequence shown in Table 6C. An ORP was identified beginning with an ATG initiation codon at nucleotides 499-501 and ending with a TAA codon at nucleotides 1300-1302. A putative untranslated region upstream from the initiation codon and downstream from the termination codon is underlined in Table 6C, and the start and stop codons are in bold letters.

TABLE 6C
FCTR6b Nucleotide Sequence (SEQ ID NO:22)

GATTTTAGAAGGTTAATCAAAAACCCGGGGACAGTTTCTTCATGGCATAACCACAGACCTTTGTGGCACCCGCTGT
CGTGGGATATCAAATATCCTCTGGGGTTCGGAATGTGGGCTTATTACTGAAGATCCTGTCTGCTTGGTCAGTGGCAGGTC
TAGACTAACTTCTGGTCCTGAGTTTCTAAAGTGCTGGTAGACCAGTTGATACAAAACAGATATAATAATGAATGCCTTAT
CTATCTGAAGGTCAGTTTGATCCGTGCCAAGTGGCTTTTTGTGGGCTGTGTAGAGTGCTCTAAACCCAGCTCGGCCTTTG
CTGTATTAGACAGAAGCACCTCATTCATATCCCTGGCGCCCCTGATGGTGCAGTGGTCTGGCTGTGGTCTGCACACCAGC
TATTCTGTTTTGTTTTGTTTTGTTTTGTTTTTTCCTACCTTTTTCCAATCCTCACACCTTCTGATCAACAGCCCCAGTAG
GGTTTAAAGGTCCTAGAGCTACATGGGATTTAGGTTTCTGGGCACAGCCAATTCTGCCACTTTTGAGACTTCCCTTCCCC
TTCCACTTGCCCCTCTCTCGTTCTCTGCCACCAGTCCAGAAGAACTGAGTGTCGTGCTGGGGACCAACGACTTAACTAGC
CCATCCATGGAAATAAAGGAGGTCGCCAGCATCATTCTTCACAAAGACTTTAAGAGAGCCAACATGGACAATGACATTGC
CTTGCTGCTGCTGGCTTCGCCCATCAAGCTCGATGACCTGAAGGTGCCCATCTGCCTCCCCACGCAGCCCGGCCCTGCCA
CATGGCGCGAATGCTGGGTGGCAGGTTGGGGCCAGACCAATGCTGCTGACAAAAACTCTGTGAAAACGGATCTGATGAAA
GTGCCAATGGTCATCATGGACTGGGAGGAGTGTTCAAAGATGTTTCCAAAACTTACCAAAAATATGCTGTGTGCCGGATA
CAAGAATGAGAGCTATGATGCCTGCAAGGGTGACAGTGGGGGGCCTCTGGTCTGCACCCCAGAGCCTGGTGAGAAGTGGT
ACCAGGTGGGCATCATCAGCTGGGGAAAGAGCTGTGGAGAGAAGAACACCCCAGGGATATACACCTCGTTGGTGAACTAC
AACCTCTGGATCGAGAAAGTGACCCAGCTAGAGGGCAGGCCCTTCAATGCAGAGAAAAGGAGGACTTCTGTCAAACAGAA
ACCTATGGGCTCCCCAGTCTCGGGAGTCCCAGAGCCAGGCAGCCCCAGATCCTGGCTCCTGCTCTGTCCCCTGTCCCATG
TGTTGTTCAGAGCTATTTTGTACTGATAATAAAATAGAGGCTATTCTTTCAACCGAAA

The FCTR6b protein encoded by SEQ ID NO:22 has 267 amino acid residues and is presented using the one-letter code in Table 6B. The Psort profile for FCTR4 predicts that this sequence has no N-terminal signal peptide and is likely to be localized at the mitochondrial matrix space (Certainty=0.4372). The predicted molecular weight of this protein is 29498.9 Daltons.

TABLE 6D
Encoded FCTR6b protein sequence (SEQ ID NO:23).

MGFRFLGTANSATFETSLPLPLAPLWFSATSPEELSVVLGTNDLTSPSMEIKEVASIILHKDFKRANMDNDIALLLLASPIKL
DDLKVPICLPTQPGPATWRECWVAGWGQTNAADNSVKTDLMKVPMVIMDWEECSKMFPKLTKNMLCAGYKNESYDACKGDSG
GPLVCTPEPGEKWYQVGIISWGKSCGEKTPGIYTSLVNYNLWIEKVTQLEGRPFNAEKRRTSVKQKPMGSPVSGVPEPGSPR
SWLLLCPLSHVLFRAILY

In a search of sequence databases, it was found, for example, that the FCTR6a nucleic acid sequence has 853 of 897 bases (95 %) identical to bases 551-1447, and 346 of 388 bases (89%) identical to bases 127-513 of Macaca fascicularis brain cDNA, clone QccE-17034 (GENBANK-ID: AB046651) (Table 6E).

TABLE 6E
BLASTN of FCTR6a against Macaca fascicularis brain cDNA,
clone QccE-17034 (SEQ ID NO:82)

>GI|9651112|DBJ|AB046651.1|AB046651 MACACA FASCICULARIS BRAIN CDNA,
CLONE QCCE-17034
LENGTH = 1746
SCORE = 1429 BITS (721), EXPECT = 0.0
IDENTITIES = 853/897 (95%)
STRAND = PLUS/PLUS

| | | |
|---|---|---|
| QUERY: 434 | CCTTTTTCCAATCCTCACACCTTCTGATCAACAGCCCCAGTAGGGTTTAAAGGTCCTAGA | 493 |
| | \|\|\|\|\|\|\|\|\|\|\|\|\|\|\|\|\|\|\|\|\|\|\|\|\|\|\| \| \|\|\|\|\|\|\|\|\|\|\|\|\|\|\|\| \|\|\|\| \|\|\|\|\|\|\|\| | |
| SBJCT: 551 | CCTTTTTCCAATCCTCACACCTTCTGAGCTACAGCCCCAGTAGGGTCTAAATGTCCTAGA | 610 |
| QUERY: 494 | GCTACATGGGATTTAGGTTTCTGGGCACAGCCAATTCTGCCACTTTTGAGACTTCCCTTC | 553 |
| | \|\|\|\| \|\|\| \|\|\|\|\|\|\|\|\|\|\|\|\|\| \|\|\|\|\|\|\|\|\|\|\|\|\|\| \|\|\|\|\|\|\|\|\|\|\| \|\|\|\|\|\|\|\|\| | |
| SBJCT: 611 | GCTATATGAGATTTAGGTTTCTGAGCACAGCCAATTCTCCCACTTTTGAGCCTTCCCTTC | 670 |
| QUERY: 554 | CCCTTCCACTTGCCCCTCTCTGGTTCTCTGCCACCAGTCCAGAAGAACTGAGTGTCGTGC | 613 |
| | \|\|\|\|\| \|\|\|\| \|\|\|\|\|\|\|\|\|\|\|\|\|\|\|\|\|\|\|\|\|\|\|\|\|\|\|\|\|\|\|\|\|\|\|\|\|\|\|\| \|\|\|\|\|\|\|\| | |
| SBJCT: 671 | CCCTTTCACTCGCCCCTCTCTGGTTCTCTGCCACCAGTCCAGAAGAACTGAATGTCGTGC | 730 |
| QUERY: 614 | TGGGGACCAACGACTTAACTAGCCCATCCATGGAAATAAAGGAGGTCGCCAGCATCATTC | 673 |
| | \|\|\|\|\|\|\|\|\|\|\|\|\|\|\|\|\|\|\|\|\|\|\| \|\|\|\|\|\|\|\|\|\|\|\|\|\|\|\|\|\|\|\|\|\|\|\|\|\|\|\|\|\|\|\|\|\|\|\| | |
| SBJCT: 731 | TGGGGACCAACGACTTAACTAGCTCATCCATGGAAATAAAGGAGGTCGCCAGCATCATTC | 790 |
| QUERY: 674 | TTCACAAAGACTTTAAGAGAGCCAACATGGACAATGACATTGCCTTGCTGCTGCTGGCTT | 733 |
| | \|\|\|\|\|\|\| \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| \| | |
| SBJCT: 791 | TTCACAAGGACTTTAAGAGAGCCAACATGGACAATGACATTGCCTTGCTGCTGCTGGCCT | 850 |
| QUERY: 734 | CGCCCATCAAGCTCGATGACCTGAAGGTGCCCATCTGCCTCCCCACGCAGCCCGGCCCTG | 793 |
| | \|\|\|\|\|\|\|\|\| \|\|\|\|\|\|\|\|\|\|\|\|\|\|\|\|\|\|\|\|\|\|\|\|\|\|\|\|\|\|\|\|\| \|\|\|\|\|\|\| \|\|\|\|\|\| \| | |
| SBJCT: 851 | CGCCCATCACACTCGATGACCTGAAGGTGCCCATCTGCCTCCCTACGCAGCACGGCCCCG | 910 |
| QUERY: 794 | CCACATGGCGCGAATGCTGGGTGGCAGGTTGGGGCCAGACCAATGCTGCTGACAAAAACT | 853 |
| | \|\|\|\|\|\|\|\|\| \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: 911 | CCACATGGCACGAATGCTGGGTGCCAGGTTGGGGCCAGACCAATGCTGCTGACAAAAACT | 970 |
| QUERY: 854 | CTGTGAAAACGGATCTGATGAAAGTGCCAATGGTCATCATGGACTGGGAGGAGTGTTCAA | 913 |
| | \|\|\|\|\|\|\|\|\|\|\|\|\|\|\|\|\|\|\|\|\|\|\|\| \|\|\| \|\|\|\|\|\|\|\|\|\|\|\|\|\|\|\|\|\|\|\|\|\|\|\|\|\|\|\|\|\|\| | |
| SBJCT: 971 | CTGTGAAAACGGATCTGATGAAAGCGCCGATCGTCATCATGGACTGGGAGGAGTGTTCAA | 1030 |
| QUERY: 914 | AGATGTTTCCAAAACTTACCAAAAATATGCTGTGTGCCGGATACAAGAATGAGAGCTATG | 973 |
| | \|\| \|\|\|\|\|\|\|\|\|\|\|\| \|\|\|\|\|\|\|\|\|\|\|\|\|\|\|\|\|\|\|\| \|\|\|\|\|\|\|\| \|\|\|\|\|\|\|\|\|\|\|\|\| | |
| SBJCT: 1031 | AGGCGTTTCCAAAACTCACCAAAAATATGCTGTGTGCTGGATACAATAATGAGAGCTATG | 1090 |
| QUERY: 974 | ATGCCTGCAAGGGTGACAGTGGGGGGCCTCTGGTCTGCACCCCAGAGCCTGGTGAGAAGT | 1033 |
| | \| \|\|\|\|\|\| \|\|\|\|\|\|\|\|\|\| \|\|\|\|\| \|\|\|\|\|\|\|\|\|\|\|\|\|\|\|\|\|\|\|\|\|\|\|\|\|\|\|\|\|\|\|\|\|\| | |
| SBJCT: 1091 | ACGCCTGCCAGGGTGACAGCGGGGGACCTCTGGTCTGCACCCCAGAGCCTGGTGAGAAGT | 1150 |
| QUERY: 1034 | GGTACCAGGTCGGCATCATCAGCTGGGGAAAGAGCTGTGGAGAGAAGAACACCCCAGGGA | 1093 |
| | \|\|\|\|\|\|\|\|\|\|\|\|\| \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: 1151 | GGTACCAGGTGGGTATCATCAGCTGGGGAAAGAGCTGTGGAGAGAAGAACACCCCAGGGA | 1210 |
| QUERY: 1094 | TATACACCTCGTTGGTGAACTACAACCTCTGGATCGAGAAAGTGACCCAGCTAGAGGGCA | 1153 |
| | \|\|\|\|\|\|\|\|\|\|\|\|\|\|\|\|\|\|\|\|\|\|\|\|\|\|\|\|\|\|\|\|\|\|\|\|\|\|\|\| \|\|\|\|\|\|\|\|\|\|\|\|\|\|\|\|\|\|\| | |
| SBJCT: 1211 | TATACACCTCGTTGGTGAACTACAACCTCTGGATCGAGAAGGTGACCCAGCTAGAGGGCA | 1270 |
| QUERY: 1154 | GGCCCTTCAATGCAGAGAAAAGGAGGACTTCTGTCAAACAGAAACCTATGGGCTCCCCAG | 1213 |
| | \|\|\|\|\|\|\|\|\| \|\|\| \|\|\|\|\|\|\| \|\|\|\|\|\| \|\|\|\|\|\|\|\|\|\|\|\|\|\|\|\|\|\|\|\|\|\|\|\|\|\|\|\| \|\| | |
| SBJCT: 1271 | GGCCCTTCAGTGCGGAGAAAATGAGGACCTCTGTCAAACAGAAACCTATGGGCTCCCGAG | 1330 |
| QUERY: 1214 | TCTCGGGAGTCCCAGAGCCAGGCAGCCCCAGATCCTGGCTCCTGCTCTGTCCCCTGTCCC | 1273 |
| | \|\|\|\|\|\|\| \|\|\|\|\|\|\|\|\|\|\|\|\|\|\| \|\|\| \|\|\|\|\|\|\|\|\|\|\|\|\|\|\|\|\|\|\|\|\|\|\|\|\|\|\|\|\|\|\|\| | |
| SBJCT: 1331 | TCTCGGGGGTCCCAGAGCCAGGCGGCCTCAGATCCTGGCTCCTGCTCTGTCCCCTGTCCC | 1390 |
| QUERY: 1274 | ATGTGTTGTTCAGAGCTATTTTGTACTGATAATAAAATAGAGGCTATTCTTTCAACC | 1330 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: 1391 | ATGTGTTGTTCAGAGCTATTTTGTACTGATAATAAAATAGACGCTATTTTTTTAACC | 1447 |

SCORE = 428 BITS (216), EXPECT = E-117
IDENTITIES = 346/388 (89%), GAPS = 1/388 (0%)
STRAND = PLUS/PLUS

| | | |
|---|---|---|
| QUERY: 1 | GATTTTAGAAGGTTAATCAAAAACCCGGGGACAGTTTCTTCATGGCATAACCACAGACCT | 60 |
| | \|\|\|\|\|\|\|\|\|\|\|\|\|\|\|\|\|\|\|\|\|\|\|\|\|\| \|\|\|\|\|\|\|\|\|\| \|\|\|\|\| \|\|\|\|\|\|\|\| \|\|\|\|\| | |
| SBJCT: 127 | GATTTTAGAAGGTTAATCAAAAACCCAAGGACAGTTTCATCATGTCATAACCAAAGACCC | 186 |
| QUERY: 61 | TTGTGGCACCCGCTGTCGTGGGATATCAAATATCCTCTGGGGTTCGGAATGTGGGCTTAT | 120 |
| | \|\|\|\|\|\|\|\|\|\| \|\|\|\|\|\| \|\|\|\|\|\|\| \|\|\|\|\|\|\|\| \| \|\|\|\|\|\|\|\| \|\|\|\|\|\|\|\| \|\|\|\|\| | |
| SBJCT: 187 | TTGTGGCACCTGCTGTCATGGGATAACAAATATCTTGTGGGGTTCTGAATGTGGACTTAT | 246 |
| QUERY: 121 | TACTGAAGATCCTGTCTGCTTGGTCAGTGGCAGGTCTAGACTAACTTCTGGTCCTGAGTT | 180 |
| | \|\|\|\|\|\|\|\| \|\|\|\|\|\|\|\|\|\|\|\|\|\|\|\|\|\|\|\|\| \|\|\|\|\|\|\|\|\|\|\|\|\|\|\|\|\|\|\|\|\|\|\|\|\|\|\| \| | |
| SBJCT: 247 | TACTGAAGCTCCTGTCTGCTTGG-TCAGTGGTGGTCTAGACTAACTTCTGGTCCTGAGAT | 305 |
| QUERY: 181 | TCTAAAGTGCTGGTAGACCAGTTGATACAAAACAGATATAATAATGAATGCCTTATCTAT | 240 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: 306 | TCTAAAGTGTTCGTAGACCGGTTGAGATAAAAGATATATAATAATGAATGCCTTACCTAT | 365 |
| QUERY: 241 | CTGAAGGTCAGTTTGATCCGTGCCAAGTGGCTTTTTGTGGGCTGTGTAGAGTGCTCTAAA | 300 |
| | \|\|\|\|\| \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| SBJCT: 366 | CTGAAAACCAGTTTGATCCGTGCCAAGGGGCTTTTTGTGGGCTCTGTAGAGTGCCCTAAA | 425 |
| QUERY: 301 | CCCAGCTCGGCCTTTGCTGTATTAGACAGAAGCACCTCATTCATATCCCTGGGGCCCCTG | 360 |
| | \|\|\|\|\|\|\|\|\|\|\|\|\|\|\|\|\|\|\|\|\|\|\|\|\|\|\|\|\|\|\|\|\| \|\|\|\|\|\|\|\|\|\|\|\|\|\|\|\|\|\|\| | |
| SBJCT: 426 | CCCAGCTCTGCCTTTGCTGTGTTAGACAGAAGCACGCCATTCACATCTCTGGGGCCCCCA | 485 |
| QUERY: 361 | ATGGTGCAGTGGTCTGGCTGTGGTCTGC | 388 |
| | \|\|\|\|\|\|\| \|\|\|\|\|\|\|\|\|\|\|\|\|\|\|\|\| | |
| SBJCT: 486 | ATGGTGCCATGGTGTGGTTGTGGTCTGC | 513 |

In a search of sequence databases, it was found, for example, that the FCTR6a nucleic acid sequence has 295 of 378 bases (78 %/O) identical to bases 410-779 of Mus musculus adult male testis cDNA, RIKEN full-length enriched (GENBANK-ID:AK09660) (Table 6F).

TABLE 6F
BLASTN of FCTR6a against Mus musculus adult male testis cDNA, RIKEN
full-length enriched (SEQ ID NO:83)

```
>GI|12855429|DBJ|AK016601.1|AK016601 MUS MUSCULUS ADULT MALE
TESTIS CDNA, RIKEN FULL-LENGTH ENRICHED
                          LIBRARY, CLONE:4933401F05, FULL INSERT SEQUENCE
                          LENGTH = 1047
SCORE = 97.6 BITS (49), EXPECT = 2E-17
IDENTITIES = 295/378 (78%), GAPS = 8/378 (2%)
STRAND = PLUS/PLUS
```

| | | |
|---|---|---|
| QUERY: 697 | AACATGGACAATGACATTGCCTTGCTGCTGCTGGCTTCGCCCATCAAGCTCGATGACCTG | 756 |
| | \|\|\|\|\|\|\|\|\|\|\| \|\|\|\|\|\|\|\|\|\|\|\| \|\|\|\|\|\|\| \|\| \|\|\|\| \| \| \| \|\| \|\|\|\| \|\|\| | |
| SBJCT: 410 | AACATGGACAACGACATTGCCTTGTTGCTGCTAGCCAAGCCCTTGACGTTCARTGAGCTG | 469 |
| QUERY: 757 | AAGGTGCCCATCTGCCTCCCCACGCAGCCCGGCCCTGCCACATGGCGCGAATGCTGGGTG | 816 |
| | \| \|\|\|\|\|\|\|\|\|\|\|\|\|\|\| \|\| \|\|\|\|\| \|\|\|\| \|\|\| \|\|\|\| \|\|\|\|\|\|\|\|\|\|\|\|\| | |
| SBJCT: 470 | ACGGTGCCCATCTGCCTTCCTCTCTGGCCCGCCCCTCCCAGCTGGCACGAATGCTGGGTG | 529 |
| QUERY: 817 | GCAGGTTGGGGCCAGACCAATGCTGCTGACAAAAACTCTGTGAAAACGGATCTGATGAAA | 876 |
| | \|\|\|\|\| \|\|\|\|\|\| \|\|\|\|\| \| \|\|\|\|\|\|\| \| \|\|\| \|\| \|\|\|\|\|\|\|\|\|\|\|\|\|\|\| | |
| SBJCT: 530 | GCAGGATGGGGCGTAACCAACTCAACTGACAAGGAATCTATGTCAACGGATCTGATGAAG | 589 |
| QUERY: 877 | GTGCCAATGGTCATCATGGACTGGGAGGAGTGTTCAAAGATGTTTCCAAAACTTACCAAA | 936 |
| | \|\|\|\|\| \|\|\| \|\|\|\|\| \|\| \|\|\|\|\|\|\|\| \|\| \| \| \|\|\|\|\|\|\|\|\|\| \| \|\| \|\|\|\| \| | |
| SBJCT: 590 | GTGCCCATGCGTATCATAGAGTGGGAGGAATGCTTACAGATGTTTCCCAGCCTCACCACA | 649 |
| QUERY: 937 | AATATGCTGTGTGCCGGATACAAGAATGAGAGCTATGATGCCTGCAAGGGTGACAGTGGG | 996 |
| | \|\| \|\|\|\|\|\|\|\|\|\|\|\| \|\|\| \|\|\|\|\|\|\|\|\|\|\| \|\|\|\|\| \|\|\| \|\|\|\|\|\|\| | |
| SBJCT: 650 | AACATGCTGTGTGCCTCATATGGTAATGAGAGCTACGATGCTTGC--------CAGTGGG | 701 |
| QUERY: 997 | GGGCCTCTGGTCTGCACCCCAGAGCCTGGTGAGAAGTGGTACCAGGTGGGCATCATCAGC | 1056 |
| | \|\| \|\| \|\| \|\|\|\|\|\|\|\|\| \|\|\|\| \|\|\|\|\| \| \|\|\|\|\|\|\|\|\|\|\|\|\|\|\|\|\|\|\|\|\|\|\|\|\| | |
| SBJCT: 702 | GGACCGCTTGTCTGCACCACAGATCCTGGCAGTAGGTGGTACCAGGTGGGCATCATCAGC | 761 |
| QUERY: 1057 | TGGGGAAAGAGCTGTGGA | 1074 |
| | \|\|\|\|\| \|\|\|\|\|\|\|\|\|\|\|\| | |
| SBJCT: 762 | TGGGGCAAGAGCTGTGGA | 779 |

The FCTR6a amino acid has 247 of 267 amino acid residues (92%) identical to, and 251 of 307 residues (94%) positive with, the 267 amino acid hypothetical protein [Macaca fascicularis] (GenBank: AB046651) (SEQ ID NO:84) (Table 6G).

TABLE 6G
BLASTP of FCTR6a and b against hypothetical protein [Macaca fascicularis]
(SEQ ID NO:84)

>GI|9651113|DBJ| BAB03569.1| (AB046651) HYPOTHETICAL PROTEIN
[MACACA FASCICULARIS]
LENGTH = 267
SCORE = 467 BITS (1202), EXPECT = E-131
IDENTITIES = 247/267 (92%), POSITIVES = 251/267 (94%)

| | | |
|---|---|---|
| QUERY: 1 | MGFRFLGTANSATFETSLPLPLAPLWFSATSPEELSVVLGTNDLTSPSMEIKEVASIILH | 60 |
| | \| \|\|\|\| \|\|\|\| \|\|\| \|\|\|\| \|\|\|\|\|\|\|\|\|\|\|\|\|\|+\|\|\|\|\|\|\|\|\|\| \|\|\|\|\|\|\|\|\|\|\|\|\| | |
| SBJCT: 1 | MRFRFLSTANSPTFEASLPLSLAPLWFSATSPEELNVVLGTNDLTSSSMEIKEVASIILH | 60 |
| QUERY: 61 | KDFKRANMDNDIALLLLASPIKLDDLKVPICLPTQPGPATWRECWVAGWGQTNAADKNSV | 120 |
| | \|\|\|\|\|\|\|\|\|\|\|\|\|\|\|\|\|\|\|\|\| \|\|\|\|\|\|\|\|\|\|\|\|\| \|\|\|\|\| \|\|\|\|\|\|\|\|\|\|\|\|\|\|\|\|\|\| | |
| SBJCT: 61 | KDFKRANMDNDIALLLLASPITLDDLKVPICLPTQEGPATWHECWVAGWGQTNAADKNSV | 120 |
| QUERY: 121 | KTDLMKVPMVIMDWEECSKMFPKLTKNMLCAGYKNESYDACKGDSGGPLVCTPEPGEKWY | 180 |
| | \|\|\|\|\|\| \|\|\|\|\|\|\|\|\|\|\|\| \|\|\|\|\|\|\|\|\|\|\|\|\| \|\|\|\|\|\|\|+\|\|\|\|\|\|\|\|\|\|\|\|\|\|\|\|\|\| | |
| SBJCT: 121 | KTDLMKAPMVIMDWEECSKAFPKLTKNNLCAGYNNESYDACQGDSGGPLVCTPEPGEKWY | 180 |
| | K E | |
| QUERY: 181 | QVGIISWGKSCGDKNTPGIYTSLVNYNLWIEKVTQLGGRPFNAEKRRTSVKQKPMGSPVS | 240 |
| | \|\|\|\|\|\|\|\|\|\|\|\|+\|\|\|\|\|\|\|\|\|\|\|\|\|\|\|\|\|\|\|\|\|\|\| \|\|\|\|+\|\|\| \|\|\|\|\|\|\|\|\|\|\| \|\| | |
| SBJCT: 181 | QVGIISWGKSCGEENTPGIYTSLVNYNLWIEKVTQLEGRPFSAEKMRTSVKQKPMGSRVS | 240 |
| QUERY: 241 | GVPEPGSPRSWLLLCPLSEVLFRAILY | 267 |
| | \|\|\|\|\|\|\| \|\|\|\|\|\|\|\|\|\|\|\|\|\|\|\|\|\|\| | |
| SBJCT: 241 | GVPEPGGLRSWLLLCPLSEVLFRAILY | 267 |

K AND E ARE RESIDUES THAT DIFFER BETWEEN FCTR6A AND B. D193K, AND G217E.

The FCTR6a amino acid has 80 of 201 amino acid residues (39%) identical to, and 119 of 201 residues (58%) positive with, the 638 amino acid plasma kallikrein B1 precursor (GENBANK-ID:NP_{—}000883.1) (SEQ ID NO:85) (Table 6H).

TABLE 6H
BLASTP of FCTR6a and b against plasma kallikrein B1 precursor
(SEQ ID NO:85)

```
>GI|4504877|REF|NP_000883.1| PLASMA KALLIKREIN B1 PRECURSOR;
                      KALLIKREIN, PLASMA; KALLIRREIN B
                      PLASMA; KALLIRREIN 3, PLASMA; FLETCHER FACTOR [HOMO SAPIENS]
GI|125184|SP|P03952|KAL HUMAN PLASMA KALLIEREIN PRECURSOR
(PLASMA PREKALLIRREIN) (KININOGENIN)
                      (FLETCHER FACTOR)
GI|67591|PIR||KQHUP PLASMA KALLIKREIN (EC 3.4.21.34) PRECURSOR - HUMAN
GI|190263|GB|AAA60153.1| (M13143) PLASMA PREKALLIEREIN [HOMO SAPIENS
GI|8809781|GB|AAF79940.1| (AF232742) PLASMA KALLIKREIN PRECURSOR
[HOMO SAPIENS]
                  LENGTH 638
SCORE = 133 BITS (334), EXPECT = 3E-30
IDENTITIES = 80/201 (39%), POSITIVES = 119/201 (58%), GAPS = 18/201 (8%)
```

| | | |
|---|---|---|
| QUERY: 20 | LPLAPLWFSATSPEELSVVLGTNDLT--SPSMEIREVASIILHKDFKRANMDNDIALLLL | 77 |
| | \|\|\| +\| \| +\| +\|+\| +\| +\|\|\| \|\|+\|+++\| + ++\|\|\|\|+ \| | |
| SBJCT: 439 | LPLQDVW------RIYSGILNLSDITKDTPFSQIKE---IIIEQNYKVSEGNHDIALIKL | 489 |
| QUERY: 78 | ASPIKLDDLKVPICLPTQPGPAT-WRECWVAGWGQTNAADKNSVKTDLMKVPMVIMDWEE | 136 |
| | +\|+ + + \|\|\|\|\|++ +\| + \|\|\| \|\|\| + +\| ++ \| \|\| + ++ \|\| | |
| SBJCT: 490 | QAPLNYTEFQKPICLPSKGDTSTIYTNCWVTGWGFSK--EKGEIQNILQKVNIPLVTNRE | 547 |
| QUERY: 137 | CSKMFP--KLTKNMLCAGYKNESYDACKGDSGGPLVCTPEPGEKWYQVGIISWGKSCGDK | 194 |
| | \| \| + \|+\|+ \|+\|\|\|\|\| \|\|\|\|\|\|\|\|\|\|\|\|\| + \| \|\|\| \|\|\|+ \| + | |
| SBJCT: 548 | CQKRYQDYKITQRMVCAGYKEGGKDACKGDSGGPLVC--KHNGMWRLVGITSWGEGCARR | 605 |
| QUERY: 195 | NTPGIYTSLVNYNLWIEKVTQ | 215 |
| | \|\|+\|\| + \| \|\| + \|\| | |
| SBJCT: 606 | EQPGVYTKVAEYMDWILEKTQ | 626 |

K IS A RESIDUE THAT DIFFERS BETWEEN FCTR6A AND B. D193K.

The FCTR6a amino acid has 73 of 183 amino acid residues (39%) identical to, and 110 of 183 residues (59%) positive with, the 643 amino acid kallikrein [Sus scrofa] (GENBANK-ID:BAA37147.1) (SEQ ID NO:86) (Table 61).

TABLE 61
BLASTP of FCTR6a and b against kallikrein [Sus scrofa]
(SEQ ID NO:86)

>GI|4165315|DBJ|BAA371471.1| (AB022425) KALLIRREIN [SUS SCROFA]
LENGTH = 643
SCORE = 128 BITS (322), EXPECT = 9E-29
IDENTITIES = 73/183 (39%), POSITIVES = 110/183 (59%), GAPS = 12/183 (6%)

| | | |
|---|---|---|
| QUERY: 38 | VLGTNDLT--SPSMEIKEVASIILEKDFKRANMDNDIALLLLASPIKLDDLKVPICLPTQ | 95 |
| | +\| +++\| +\| ++\|\| \|\|+\|+++\| +\|\|\|\|\| \|+\|+ \| + \|\|\|\|\|++ | |
| SBJCT: 459 | ILNISEITKETPFSQVKE---IIIHQNYKILESGHDIALLKLETPLNYTDFQKPICLPSR | |
| QUERY: 96 | PGP-ATWRECWVAGWGQTNAADKNSVKTDLMKVPMVIMDWEECSKMFP--KLTKNMLCAG | 152 |
| | + \|\|\| \|\|\| \| +\| ++ \| \|\| + ++ \|\|\| \| + \|++\| \|+\|\|\| | |
| SBJCT: 516 | DDTNVVYTNCWVTGWGFTE--EKGEIQNILQKVNIPLVSNEECQKSYPLEKISKQMICAG | 573 |
| QUERY: 153 | YKNESYDACKGDSGGPLVCTPEPGEKWYQVGIISWGKSCGDKNTPGIYTSLVNYNLWIEK | 212 |
| \|\| \|\|\|\|\|+\|\|\|\|\|\|\| + \|+ \|\| \|\|\|+ \| + \|\|+\|\| ++ \| \|\| + | | |
| SBJCT: 574 | YKEGGKDACKGESGGPLVC--KYNGIWHLVGTTSWGEGCARREQPGVYTKVIEYMDWILE | 631 |
| QUERY: 213 | VTQ | 215 |
| | \|\| | |
| SBJCT: 632 | KTQ | 634 |

K IS A RESIDUE THAT DIFFERS BETWEEN FCTRGA AND B. D193K.

The FCTR6a amino acid has 81 of 205 amino acid residues (39%) identical to, and 112 of 205 residues (54%) positive with, the 625 amino acid Coagulation factor XI [Homo sapiens] (embCAA64368.1) (SEQ ID NO:87) (Table 6J).

TABLE 6J
BLASTP of FCTR6a and b against Coagulation factor XI [Homo sapiens]
(SEQ ID NO:87)

>GI|180352|GB|AAA51985.1| (M20218) COAGULATION FACTOR XI
[HOMO SAPIENS]
LENGTH = 625
SCORE = 127 BITS (320), EXPECT = 1E-28
IDENTITIES = 81/205 (39%), POSITIVES = 112/205 (54%),
GAPS = 17/205

| | | |
|---|---|---|
| QUERY: 20 | LPLAPLWFSATSPEELSVVLGTNDLTSPSMEIKE------VASIILHKDFKRANMDNDIA | 73 |
| | \| \| ++ \|\|+ \| \| \| + + \|\|\|\| \| \|\|+\| +\| \| \|\|\| | |
| SBJCT: 427 | LTAAHCFYGVESPKILRVYSGILNQS----EIKEDTSFFGVQEIIIEDQYKMAESGYDIA | 482 |
| QUERY: 74 | LLLLASPIKLDDLKVPICLPTQPG-PATWRECWVAGWGQTNAADKNSVKTDLMKVPMVIM | 132 |
| | \|\| \| + + \| + \|\|\|\|\|++ + +\|\|\| \|\|\| \|\| ++ \| \| + ++ | |
| SBJCT: 483 | LLKLETTVNYTDSQRPICLPSKGDRNVIYTDCWVTGWGYRKLRDK--IQNTLQKAKIPLV | 540 |
| QUERY: 133 | DWEECSKMFP--KLTKNMLCAGYKNBSYDACKGDSGGPLVCTPEPGEKWYQVGIISWGKS | 190 |
| | \|\|\| \| + \|+\| \|+\|\|\|\|+ \|\|\|\|\|\|\|\|\|\|\| \| + \| \|+ \|\|\| \|\|\|+ | |
| SBJCT: 541 | TNEECQKRYRGEKITHKMICAGYREGGKDACKGDSGGPLSC--KHNEVWHLVGITSWGEG | 598 |
| QUERY: 191 | CGDKNTPGIYTSLVNYNLWIEKVTQ | 215 |
| | \| + \|\|+\|\|++\| \| \|\| + \|\| | |
| SBJCT: 599 | CAQRERPGVYTNVVEYVDWILEKTQ | 623 |

K IS A RESIDUE THAT DIFFERS BETWEEN FCTR6A AND B. D193K.

The number of new cases of renal cell carcinoma in the United States in 1996 was projected to be 30,600 with an estimated 12,000 deaths. Tumors with a proposed histogenesis from the proximal tubule (clear-cell and chromophilic tumors) amount to 85% of renal cancers, whereas tumors with a proposed histogenesis from the connecting tubule/collecting duct (chromophobic-, oncocytic-, and duct Bellini-type tumors) amount to only 11%.

Adenocarcinomas may be separated into clear cell and granular cell carcinomas, although the 2 cell types may occur together in some tumors. The distinction between well-differentiated renal carcinomas and renal adenomas can be difficult. The diagnosis is usually made arbitrarily on the basis of size of the mass, but size alone should not influence the treatment approach, since metastases can occur with lesions as small as 0.5 centimeters.

While radical nephrectomy with regional lymphadenectomy, is the accepted, often curative therapy for stage I (localized disease) renal cell cancer, very little therapy is available for advance disease that represent about 70% of the patients. Radiotherapy as a postoperative adjuvant has not been effective, and when used preoperatively, may decrease local recurrence but does not appear to improve 5-yr survival. A chemotherapeutic agent capable of significantly altering the course of metastastic renal cell carcinoma has not been identified. (Renal Cell Cancer (PDQ®) Treatment—Health Professionals, Cancemet, NCI) There is therefore a need to identify genes that are differentially modulated in renal-cell carcinomas. In addition there is a need for methods to assay candidate therapeutic substances for modulating expression of these genes. These substances might be recombinant protein expressed by the identified genes or antibodies that bind to the identified proteins. There is yet additionally a need for an effective method of identifying target molecules or related components. These and related needs and defects are addressed in the present invention.

Novel kallikrein-like/coagulation factor XI-like Proteins and Nucleic Acids Encoding Same

FCTR6 is surprisingly found to be differentially expressed in clear cell Renal cell carcinoma tissues vs the normal adjacent kidney tissues. The present invention discloses a novel protein encoded by a cDNA and/or by genomic DNA and proteins similar to it, namely, new proteins bearing sequence similarity to kallikrein-like, nucleic acids that encode these proteins or fragments thereof, and antibodies that bind immunospecifically to a protein of the invention. It may have use as a therapeutic agent in the treatment of renal cancer and liver cirrhosis.

The utility of kallikrein family members in protein therapy of Renal cancer

The treatment of renal cell carcinoma with recombinant kallikrein could improve disease outcome through several potential mechanisms. The literature suggests that members of this protein family are inhibitory to the process of angiogenesis, a process of vital importance to tumor progression. Renal cell carcinoma is known to be a highly angiogenic cancer. Thus, treatment of renal cell carcinoma with kallikrein may effectively shutdown the active recruitment of a blood supply to a tumor. Members of this protein family are known to play a role in vascular coagulation. Similar to anti-angiogenic therapy, a factor produced by cancer cells that is pro-coagulatory may also act to inhibit cancer growth by effectively “clogging” the tumor vascular supply. In addition, through its proteolytic activity, kallikrein may degrade ECM proteins or growth factors necessary for the progressive growth of cancer cells. Following is a relevant reference underlining the importance of Kallikrein in cancer therapy.

The New Human Kallikrein Gene Family: Implications in Carcinogenesis.

Diamandis E P; Yousef G M; Luo I; Magklara I; Obiezu C V

Department of Pathology and Laboratory Medicine, Mount Sinai Hospital, Toronto, Ontario, Canada.

Trends Endocrinol Metab 2000 Mar; 11(2):54-60.

ABSTRACT: The traditional human kallikrein gene family consists of three genes, namely KLK1 [encoding human kallikrein 1 (hK1) or pancreatic/renal kallikrein], KLK2 (encoding hK2, previously known as human glandular kallikrein 1) and KLK3 [encoding hK3 or prostate-specific antigen (PSA)]. KLK2 and KLK3 have important applications in prostate cancer diagnostics and, more recently, in breast cancer diagnostics. During the past two to three years, new putative members of the human kallikrein gene family have been identified, including the PRSSL1 gene [encoding normal epithelial cell-specific 1 gene (NES1)], the gene encoding zyme/protease M/neurosin, the gene encoding prostase/KLK-L1, and the genes encoding neuropsin, stratum corneum chymotryptic enzyme and trypsin-like serine protease. Another five putative kallikrein genes, provisionally named KLK-L2, KLK-L3, KLK-L4, KLK-L5 and KLK-L6, have also been identified. Many of the newly identified kallikrein-like genes are regulated by steroid hormones, and a few kallikreins (NES1, protease M, PSA) are known to be downregulated in breast and possibly other cancers. NES1 appears to be a novel breast cancer tumor suppressor protein and PSA a potent inhibitor of angiogenesis. This brief review summarizes recent developments and possible applications of the newly defined and expanded human kallikrein gene locus.

The utility of kallikrein-like/coagulation factor XI-like family members in protein therapy of liver cirrosis

Results related to inflammation shown below in Example A, Table CC3, panel 4, indicate over-expression of 27455183.0.19 in the liver cirrhosis sample, as compared to panel 1 data (Table CC1), where there is little or no expression in normal adult liver. Panel 4 was generated from various human cell lines that were untreated or resting as well as the same cells that were treated with a wide variety of immune modulatory molecules. There are several disease tissues represented as well as organ controls.

Potential Role(s) of FCTR6 in Inflammation:

Liver cirrhosis occurs in patients with hepatitis C and also in alcoholics. This protein is 41% related to coagulation factor XI and its potential role in liver cirrhosis may be related to cleavage of kininogen. A reference for this follows:

Thromb Haemost 2000 May;83(5):709-14 High molecular weight kininogen is cleaved by FXIa at three sites: Arg409-Arg4 10, Lys502-Thr503 and Lys325-Lys326. Mauron T, Lammle B, Wuillemin W A Central Hematology Laboratory, University of Bern, Inselspital, Switzerland.

Abstract:

We investigated the cleavage of high molecular weight kininogen (HK) by activated coagulation factor XI (FXIa) in vitro. Incubation of HK with FXIa resulted in the generation of cleavage products which were subjected to SDS-Page and analyzed by silver-staining, ligand-blotting and immunoblotting, respectively. Upon incubation with FXIa, bands were generated at 111, 100, 88 kDa on nonreduced and at 76, 62 and 51 kDa on reduced gels. Amino acid sequence analysis of the reaction mixtures revealed three cleavage sites at Arg409-Arg410, at Lys502-Thr5O3 and at Lys325-Lys326. Analysis of HK-samples incubated with FXIa for 3 min, 10 min and 120 min indicated HK to be cleaved first at Arg409-Arg410, followed by cleavage at Lys502-Thr503 and then at Lys325-Lys326. In conclusion, HK is cleaved by FXIa at three sites. Cleavage of HK by FXIa results in the loss of the surface binding site of HK, which may constitute a mechanism of inactivation of HK and of control of contact system activation.

Impact of Therapeutic Targeting of FCTR6 in Inflammation:

Therapeutic targeting of FCTR6 with a monoclonal antibody is anticipated to limit or block the extent of breakdown of kininogen and thereby reduce the degradation of liver that occurs in liver cirrhosis. A pertinent reference is:

Thromb Haemost 1999 November;82(5):1428-32 Parallel reduction of plasma levels of high and low molecular weight kininogen in patients with cirrhosis.

Cugno M, Scott C F, Salerno F, Lorenzano E, Muller-Esterl W, Agostoni A, Colman R W Department of Internal Medicine, IRCCS Maggiore Hospital, University of Milan, Italy. massimo.cugno@unimi.it

Abstract:

Little is known about the regulation of high-molecular-weight-kininogen (HK) and low-molecular-weight-kininogen (LK) or the relationship of each to the degree of liver function impairment in patients with cirrhosis. In this study, we evaluated HK and LK quantitatively by a recently described particle concentration fluorescence immunoassay (PCFIA) and qualitatively by SDS PAGE and immunoblotting analyses in plasma from 33 patients with cirrhosis presenting various degrees of impairment of liver function. Thirty-three healthy subjects served as normal controls. Patients with cirrhosis had significantly lower plasma levels of HK (median 49 microg/ml [range 22-99 microg/ml]) and LK (58 microg/ml [15-100 microg/ml]) than normal subjects (HK 83 microg/ml [65-115 microg/ml]; LK 80 microg/ml [45-120 microg/ml]) (p<0.0001). The plasma concentrations of HK and LK were directly related to plasma levels of cholinesterase (P<0.0001) and albumin (P<0.0001 and P<0.001) and inversely to the Child-Pugh score (P<0.0001) and to prothrombin time ratio (P<0.0001) (reflecting the clinical and laboratory abnormalities in liver disease). Similar to normal individuals, in patients with cirrhosis, plasma HK and LK levels paralleled one another, suggesting that a coordinate regulation of those proteins persists in liver disease. SDS PAGE and immunoblotting analyses of kininogens in cirrhotic plasma showed a pattern similar to that observed in normal controls for LK (a single band at 66 kDa) with some lower molecular weight forms noted in cirrhotic plasma. A slight increase of cleavage of HK (a major band at 130 kDa and a faint but increased band at 107 kDa) was evident. The increased cleavage of HK was confirmed by the lower cleaved kininogen index (CKI), as compared to normal controls. These data suggest a defect in hepatic synthesis as well as increased destructive cleavage of both kininogens in plasma from patients with cirrhosis. The decrease of important regulatory proteins like kininogens may contribute to the imbalance in coagulation and fibrinolytic systems, which frequently occurs in cirrhotic patients.

In summary, the differential expression of FCTR6 (Kallikrein family) in renal cell carcinoma is an important finding that could have immense potential in renal carcinogenesis. In addition, overexpression of the above gene in liver cirrhosis demonstrates its anticipated use as an immunotherapeutic target.

FCTR7

The novel nucleic acid of 1498 nucleotides FCTR7 (also designated. 32592466.0.64) encoding a novel trypsin inhibitor-like protein is shown in Table 7A. An ORF begins with an ATG initiation codon at nucleotides 470-472 and ends with a TAA codon at nucleotides 1369-1371. Putative untranslated regions, if any, are found upstream from the initiation codon and downstream from the termination codon.

TABLE 7A
FCTR7 Nucleotide Sequence (SEQ ID NO:24)

AGGCGCCTGGTTCTGCGCGTACTGGCTGTACGGAGCAGGAGCAAGAGGTCGCCGCCAGCCTCCGCCGCCGAGCCTCGTTCGTG
TCCCCGCCCCTCGCTCCTGCAGCTACTGCTCAGAAACGCTGGGGCGCCCACCCTGGCAGACTAACGAAGCAGCTCCCTTCCCA
CCCCAACTGCAGGTCTAATTTTGGACGCTTTGCCTGCCATTTCTTCCAGGTTGAGGGAGCCGCAGAGGCGGAGGCTCGCGTAT
TCCTGCAGTCAGCACCCACGTCGCCCCCGGACGCTCGGTGCTCAGGCCCTTCGCGAGCGGGGCTCTCCGTCTGCGGTCCCTTG
TGAAGGCTCTGGGCGGCTGCAGAGGCCGGCCGTCCGGTTTGGCTCACCTCTCCCAGGAAACTTCACACTGGAGAGCCAAAAGG
AGTGGAAGAGCCTGTCTTGGAGATTTTCCTGGGGAAATCCTGAGGTCATTCATTATGAAGTGTACCGCGCGGGAGTGGCTCAG
AGTAACCACAGTGCTGTTCATGGCTAGAGCAATTCCAGCCATGGTGGTTCCCAATGCCACTTTATTGGAGAAACTTTTGGAAA
AATACATGGATGAGGATGGTGAGTGGTGGATAGCCAAACAACGAGGGAAAAGGGCCATCACAGACAATGACATGCAGAGTATT
TTGGACCTTCATATAATTAAACGAAGTCAGGTGTATCCAACAGCCTCTAATATGGAGTATATGACATGGGATGTAGAGCTGGA
AAGATCTGCAGAATCCAGGGCTGAAATTGCTTCTGGGAACATGGACCTGCAACCTTGCTTCCATCAATTGGACAGAATTTGGG
AGCACACTGGGGAAGATATAGGCCCCCGACGTTTCATGTACAATCGTGGTATAATGAAGTGAAAGACTTTAGCTACCCATATG
AACATGAATGCAACCCATATTGTCCATTCAGGTGTTCTGGCCCTGTATGTACACATTATACACAGGTCGTGTGGCCAACTAGT
AACAGAATCGGTTGTGCCATTAATTTGTGTCATAACATGAACATCTGGGGGCAGATATGGCCCAAAGCTGTCTACCTGGTGTG
CAATTACTCCCCAAAGGGAAACTGGTGGGGCCATGCCCCTTACAAACATGGGCGGCCCTGTTCTGCTTGCCCACCTAGTTTTG
GAGGGGGCTGTAGAGAAAATCTGTGCTACAAAGAAGGGTCAGACAGGTATTATCCCCCTCGAGAAGAGGAAACAAATGAAATA
GAACCGCAGCAGTCACAAGTCCATGACACCCATGTCCGGACAAGATCAGATGATAGTAGCAGAAATGAAGTCATTAGCTTTGG
GAAAAGTAATGAAAATATAATGGTTTTAGAAATCCTGTGTTAAATATTGCTATATTTTCTTAGCAGTTATTTCTACAGTTAAT
TACATAGTCATGATTGTTCTACGTTTCATATATTATATGGTCCTTTGTATATGCCCCTAATAAAATGAATCTAAACATTGAAA
AAAA

The FCTR7 protein encoded by SEQ ID NO:24 has 300 amino acid residues and is presented using the one-letter code in Table 7B. The FCTR7 gene was found to be expressed in: brain; germ cell tumors. FCTR7 gene maps to Unigene cluster Hs.182364 which is expressed in the following tissues: brain, breast, ear, germ cell, heart, liver, lung, whole embryo, ovary, pancreas, pooled, prostate, stomach, testis, uterus, vascular. Therefore the FCTR7 protein described in this invention is also expressed in the above tissues.

The SignalP, Psort and/or Hydropathy profile for FCTR7 predict that this sequence has a signal peptide and is likely to be localized outside of the cell with a certainty of 0.4228. The SignalP shows a cleavage site between amino acids 20 and 21, i.e., at the dash in the sequence amino acid ARA-IP. The predicted molecular weight of FCTR7 is 34739.9 Daltons. Hydropathy profile shows an amino terminal hydrophobic region. This region could function as a signal peptide and target the invention to be secreted or plasma membrane localized.

TABLE 7B
Encoded FCTR7 protein sequence (SEQ ID NO:25).

NECTAREWLRVTTVLFMARAIPAMVVPNATLLEKLLEKYMDEDGEWWIAKQRGKRAITDNDMQSILDLHNKLRSQVYPTASNN
EYMTWDVELERSAESRAESCLWEHGPASLLPSIGQNLGAHWGRYRPPTFHVQSWYDEVKDFSYPYEHECNPYCPFRCSGPVCT
HYTQVVWATSNRIGCAINLCHNMNIWGQIWPKAVYLVCNYSPKGNWWGHAPYKHGRPCSACPPSFGGGCRENLCYKEGSDRYY
PFREEETNEIERQQSQVEDTHVRTRSDDSSRNEVISFGKSNENIMVLEILC

This gene maps to Unigene cluster Hs. 182364 which has been assigned the following mapping information shown in table 7C. Therefore the chromosomal assignment for this gene is the same as that for Unigene cluster 182364.

TABLE 7C
Mapping Information.

| | |
|---|---|
| Chromosome: | 8 |
| Gene Map 98: | Marker SHGC-32056, Interval D8S279-D8S526 |
| Gene Map 98: | Marker SGC32056, Interval D8S526-D8S275 |
| Gene Map 98: | Marker sts-G20223, Interval D8S526-D8S275 |
| Gene Map 98: | Marker stSG30385, Interval D8S526-D8S275 |
| Whitehead map: | EST67946, Chr.8 |
| dbSTS entries: | G25853, G29349, G20223 |

The predicted amino acid sequence was searched in the publicly available GenBank database

FCTR7 protein showed Score=743 (261.5 bits), Expect=1.4e-73, P=1.4e-73, 54% identities (129 over 237 amino acids) and 43% homologies (167 over 237 amino acids) with human 25 kD trypsin inhibitor protein (258 aa; ACC:043692) (Table 7D).

TABLE 7D
BLAST X search results are shown below:

| | |
|---|---|
| ptnr:SPTREMBL-ACC:O43692 25 KDA TRYPSIN INHIBITOR - HO. . . +2 743 8.4e-73 1 | (SEQ ID NO:88) |
| ptnr:SPTREMBL-ACC:O44228 HRTT-1 - HALOCYNTHIA RORETZI . . . +2 325 2.9e-28 1 | (SEQ ID NO:89) |
| ptnr:SWISSPROT-ACC:P48060 GLIOMA PATHOGENESIS-RELATED . . . +2 314 5.3e-27 1 | (SEQ ID NO:90) |
| ptnr:PIR-ID:JC4131 glioma pathogenesis-related protein. . . +2 309 2.0e-26 1 | (SEQ ID NO:91) |
| ptnr:SWISSNEW-ACC:O19010 CYSTEINE-RICH SECRETORY PROTE. . . +2 258 9.4e-21 1 | (SEQ ID NO:92) |

The nucleotide sequence of FCTR7 has 954 of 957 residues (99 %) identical to the 1-957 base segment, and 174 of 175 residues (99%) identical to bases 1317-1953 of the 2664 nucleotide Homo sapiens putative secretory protein precursor, mRNA (GenBank-ACC: AF142573) (SEQ ID NO:93) (Table 7E).

TABLE 7E
BLASTN of FCTR7 against Putative secretory protein precursor
(SEQ ID NO:93)

>gi|12002310|gb|AF142573.1|AF142573 Homo sapiens putative
secretory protein precursor, mRNA, complete cds
Length = 2664
Score = 1865 bits (941), Expect = 0.0
Identities = 954/957 (99%), Gaps = 1/957 (0%)
Strand = Plus/Plus

| | | |
|---|---|---|
| Query: 364 | gtccggtttggctcacctctcccaggaaacttcacactggagagccaaaaggagtggaag | 423 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: 1 | gtccggtttggctcacctctcccaggaaacttcacactggagagccaaaaggagtggaag | 60 |
| Query: 424 | agcctgtcttggagattttcctggggaaatcctgaggtcattcattatgaagtgtaccgc | 483 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: 61 | agcctgtcttggagattttcctggggaaatcctgaggtcattcattatgaagtgtaccgc | 120 |
| Query: 484 | gcgggagtggctcagagtaaccacagtgctgttcatggctagagcaattccagccatggt | 543 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: 121 | gcgggagtggctcagagtaaccacagtgctgttcatggctagagcaattccagccatggt | 180 |
| Query: 544 | ggttcccaatgccactttattggagaaacttttggaaaaatacatggatgaggatggtga | 603 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: 181 | ggttcccaatgccactttattggagaaacttttggaaaaatacatggatgaggatggtga | 240 |
| Query: 604 | gtggtggatagccaaacaacgagggaaaagggccatcacagacaatgacatgcagagtat | 663 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: 241 | gtggtggatagccaaacaacgagggaaaagggccatcacagacaatgacatgcagagtat | 300 |
| Query: 664 | tttggaccttcataataaattacgaagtcaggtgtatccaaaagcctctaatatggagta | 723 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: 301 | tttggaacttcataataaattacgaagtcaggtgtatccaacagcctctaatatggagta | 360 |
| Query: 724 | tatgacatgggatgtagagctggaaagatatgaagaatccagggctgaaa-ttgcttgtg | 782 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: 361 | tatgacatgggatgtagagctggaaagatctgcagaatcctgggctgaaagttgcttgtg | 420 |
| Query: 783 | ggaacatggaactgcaagcttgcttacatcaattggacagaatttgggagcacactgggg | 842 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: 421 | ggaacatggacctgcaagattgcttccatcaattggacagaatttgggagcacactgggg | 480 |
| Query: 843 | aagatataggcccccgacgtttcatgtacaatcgtggtatgatgaagtgaaagactttag | 902 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: 481 | aagatataggccccCgacgtttcatgtacaatcgtggtatgatgaagtgaaagactttag | 540 |
| Query: 903 | ctacccatatgaacatgaatgcaacccatattgtccattcaggtgttatggccctgtatg | 962 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: 541 | ctacccatatgaacatgaatgcaacccatattgtccattcaggtgttctggccctgtatg | 600 |
| Query: 963 | tacacattataaacaggtagtgtgggcaaatagtaacagaatcggttgtgccattaattt | 1022 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: 601 | tacaaattataaacaggtcgtgtgggcaactagtaacagaatcggttgtgcaattaattt | 660 |
| Query: 1023 | gtgtcataacatgaaaatctgggggcagatatggcccaaagctgtatacctggtgtgcaa | 1082 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: 661 | gtgtaataaaatgaacatctgggggcagatatggccaaaagatgtataactggtgtgaaa | 720 |
| Query: 1083 | ttactccacaaagggaaaatggtggggccatgccacttacaaaaatgggaggccctgtta | 1142 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: 721 | ttactccccaaagggaaactggtggggccatgccccttacaaacatgggcggccctgttc | 780 |
| Query: 1143 | tgcttgcccacctagttttggagggggctgtagagaaaatctgtgctacaaagaagggtc | 1202 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: 781 | tgcttgcccacctagttttggagggggctgtagagaaaatctgtgctacaaagaagggtc | 840 |
| Query: 1203 | agacaggtattatccccctcgagaagaggaaacaaatgaaatagaacggcagcagtcaca | 1262 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: 841 | agacaggtattatccccctcgagaagaggaaacaaatgaaatagaacgacagcagtcaca | 900 |
| Query: 1263 | agtccatgacacccatgtccggacaagatcagatgatagtagcagaaatgaagtcat | 1319 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: 901 | agtccatgacacccatgtccggacaagatcagatgatagtagcagaaatgaagtcat | 957 |

Score = 339 bits (171), Expect = 3e-90
Identities = 174/175 (99%)
Strand = Plus/Plus

| | | |
|---|---|---|
| Query: 1317 | cattagctttgggaaaagtaatgaaaatataatggttttagaaatcctgtgttaaatatt | 1376 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: 1779 | cattagctttgggaaaagtaatgaaaatataatggttttagaaatcctgtgttaaatatt | 1838 |
| Query: 1377 | gctatattttcttagcagttatttctacagttaattacatagtcatgattgttctacgtt | 1436 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: 1839 | gctatattttcttagcagttatttctacagttaattacatagtcatgattgttctacgtt | 1898 |
| Query: 1437 | tcatatattatatggtgctttgtatatgcccctaataaaatgaatctaaacattg | 1491 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: 1899 | tcatatattatatggtgctttgtatatgccactaataaaatgaatctaaacattg | 1953 |

The FCTR7 amino acid has 284 of 285 amino acid residues (99%) identical to, and 284 of 285 amino acid residues (99%) similar to, the 500 amino acid Putative secretory protein precursor [Homo sapiens] (GenBank-Acc No.: AF142573) (SEQ ID NO:94) (Table 7F).

TABLE 7F
BLASTP alignments of FCTR7 against Putative secretory
protein precursor, (SEQ ID NO:94)

>gi|12002311|gb|AAG43287.1|AF142573 1 (AF142573)
putative secretory protein precursor [Homo sapiens]
Length = 500
Score = 581 bits (1499), Expect = e-165
Identities = 284/285 (99%), Positives = 2 84/285 (99%)

| | | |
|---|---|---|
| Query: 1 | MKCTAREWLRVTTVLFMARAIPAMVVPNATLLEKLLEKYMDEDGEWWIAKQRGKRAITDN | 60 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: 1 | MKCTAREWLRVTTVLFMARAIPAMVVPNATLLEKLLEKYMDEDGEWWIAKQRGKRAITDN | 60 |
| Query: 61 | DMQSILDLUNKLRSQVYPTASNMEYMTWDVELERSAESRAESCLWEHGPASLLPSIGQNL | 120 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: 61 | DMQSILDLHNKLRSQVYPTASNMEYMTWDVELERSAESWAESCLWEHGPASLLPSIGQNL | 120 |
| Query: 121 | GAHWGRYRPPTFHVQSWYDEVKDFSYPYEHECNPYCPFRCSGPVCTHYTQVVWATSNRIG | 180 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: 121 | GAHWGRYRPPTFNVQSWYDEVKDFSYPYEHECNPYCPFRCSGPVCTNYTQVVWATSNRIG | 180 |
| Query: 181 | CAINLCHNMNIWGQIWPKAVYLVCNYSPKGNWWGHAPYKHGRPCSACPPSFCGGCRENLC | 240 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: 181 | CAINLCHNMNIWGQIWPKAVYLVCNYSPKGNWWGHAPYKHGRPCSACPPSFGGGCRENLC | 240 |
| Query: 241 | YKEGSDRYYPPREEETNEIERQQSQVHDTHVRTRSDDSSRNEVIS | 285 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: 241 | YKEGSDRYYPPREEETNEIERQQSQVHDTHVRTRSDDSSRNEVIS | 285 |

The FCTR7 amino acid has 137 of 176 amino acid residues (78%) identical to, and 151 of 176 amino acid residues (86%) similar to, the 188 amino acid Late gestation lung protein 1 [Rattus norvegicus] (GenBank-Acc No.: AF109674) (SEQ ID NO:95) (Table 7G).

TABLE 7G
BLASTP alignments of FCTR7 against Late gestation
lung protein 1, (SEQ ID NO:95)

>gi|4324682|gb|AAD16986.1| (AF109674) late gestation
lung protein 1 [Rattus norvegicus]
Length = 188
Score = 277 bits (709), Expect = 1e-73
Identities = 137/176 (78%), Positives = 151/176 (86%)

| | | |
|---|---|---|
| Query: 68 | LHNKLRSQVYPTASNMEYMTWDVELERSAESRAESCLWEHGPASLLPSIGQNLGAHWGRY | 127 |
| | \|\|\|\|\|\| \|\|\|\| \|\|\|\|\|\|\|\|\|\| \|\|\|\|\|\| \| \|\|\|\|\|\|\|\|\|\|\| \|\|\|\|\|\| \|\|\|\|\| | |
| Sbjct: 2 | LHNKLRGQVYPPASNMEYMTWDEELERSAAAWAQRCLWEHGPASLLVSIGQNLAVHWGRY | 61 |
| Query: 128 | RPPTFHVQSWYDEVKDFSYPYEHECNPYCPFRCSGPVCTHYTQVVWATSNRIGCAINLCH | 187 |
| | \| \| \|\|\|\|\|\|\|\|\|\|\|\|++\|\|\| \|\|\|\|\|+\|\| \|\|\|\| +\|\|\|\|\|\|+\|\|\|\|+\|+\|\|\|\|++ \| | |
| Sbjct: 62 | RSPGFRVQSWYDEVKDYTYPYPHECNPWCPERCSGAMCTHYTQMVWATTNKIGCAVHTCR | 121 |
| Query: 188 | NMNIWGQIWPKAVYLVCNYSPKGNWWGHAPYKHGRPCSACPPSFGGGCRENLCYKE | 243 |
| | +\|++\|\| ++ \|\|\|\|\|\|\|\|\|\|\|\|\|\| \| \|\|\|\|\|\|\|\|\|\| \|\| \|+\|\|\|\|\| \|\|\|\|+\| | |
| Sbjct: 122 | SMSVWGDIWENAVYLVCNYSPKGNWIGEAPYKHGRPCSECPSSYGGGCRNNLCYRE | 177 |

The FCTR7 amino acid has 130 of 237 amino acid residues (55%) identical to, and 165 of 237 amino acid residues (70%) similar to, the 258 amino acid R3H domain-containing preproprotein; 25 kDa trypsin inhibitor [Homo sapiens] (GenBank-Acc No.: D45027) (SEQ ID NO:96) (Table 7H).

TABLE 7H
BLASTP alignments of FCTR7 against R3H domain-containing
preproprotein, 25 kDa trypsin inhibitor (SEQ ID NO:96)

>gi|7705676|ref|NP 056970.1| R3H domain-containing
preproprotein; 25 kDa trypsin inhibitor; R3H
domain (binds single-stranded nucleic acids) containing
[Homo sapiens]
gi|2943716|dbj|BAA25066.1| (D45027) 25 kDa trypsin inhibitor
[Homo sapiens]
Length = 258
Score = 265 bits (678), Expect = 4e-70
Identities = 130/237 (55%), Positives = 165/237 (70%), Gaps = 3/237 (1%)

| | | |
|---|---|---|
| Query: 12 | TTVLFMARAIPAMVVPNATLLEKLLEKYMDEDGEWWIAKQRGKRAITDNDMQSILDLHNK | 71 |
| | +\|\|+ + + \| \| +\| \|+ +\| \| \| \| \|\| \|+ \|\|\| +\|\|\| \|\|+ | |
| Sbjct: 20 | STVVLLNSTDSSPPTNNFTDIEAALKAQLDSAD---IPKARRKRYISQNDMIAILDYHNQ | 76 |
| Query: 72 | LRSQVYPTASNMEYMTWDVELERSAESRAESCLWEHGPASLLPSIGQNLGAHWGRYRPPT | 131 |
| | +\| +\|+\| \|+\|\|\|\|\| \|\| \| \|\|\| \| \|+\|+\|\|\|+ \|\| +\|\|\|\| \|\|\|\| | |
| Sbjct: 77 | VRGKVFPPAANMEYMVWDENLAKSAEAWAATCIWDHGPSYLLRFLGQNLSVRTGRYRSIL | 136 |
| Query: 132 | FHVQSWYDEVKDFSYPYEHECNPYCPFRCSGPVCTHYTQVVWATSNRIGCAINLCHNNNI | 191 |
| | \|+ \|\|\|\|\|\|\|+++\|\| +\|\|\| \|\| \|\| \|\|+\|\|\|\|\|\|+\|\|\|\|\|\|\|\|\|\|\|\|+ \| \|\|\|+ | |
| Sbjct: 137 | QLVKPWYDEVKDYAFPYPQDCNPRCPMRCFGPMCTHYTQMVWATSNRIGCAIHTCQNMNV | 196 |
| Query: 192 | WGQIWPKAVYLVCNYSPKGNWWGHAPYKHGRPCSACPPSFGGGCRENLCYKEGSDRY | 248 |
| | \|\| +\| +\|\|\|\|\|\|\|\|+\|\|\|\|\| \| \|\|\|\| \| \|\|\|+\|\|\|\|+\|\| \| +\|\|\|+ + \| | |
| Sbjct: 197 | WGSVWRRAVYLVCNYAPKGNWIGEAPYKVGVPCSSCPPSYGGSCTDNLCFPGVTSNY | 253 |

The FCTR7 amino acid has 109 of 233 amino acid residues (47%) identical to, and 146 of 233 amino acid residues (63%) similar to, the 253 amino acid Novel protein similar to a trypsin inhibitor [Homo sapiens] 25 kDa trypsin inhibitor (ENBLAcc No.: AL117382) (SEQ ID NO:97) (Table 7I).

TABLE 71
BLASTP alignments of FCTR7 against Novel protein similar to a
trypsin inhibitor, (SEQ ID NO:97)

>gi|9885193|emb|CAC04190.1| (AL117382) dJ881L22.3
(novel protein similar to a trypsin inhibitor) [Homo sapiens]
Length = 253
Score = 225 bits (575), Expect = 4e-58
Identities = 109/233 (47%), Positives = 146/233 (63%), Gaps = 8/233 (3%)

| | | |
|---|---|---|
| Query: 10 | RVTTVLFMARAIPAMVVPNATLLEKLLEKYMDEDGEWWIAKQRGKRAITDNDMQSILDLH | 69 |
| | + \| \| \| \|\| \| +\|+ \| + + \| ++ \|+ \|\| ++\|\| \| | |
| Sbjct: 19 | QAVNALIMPNATPAPAQPESTAMRLL--------SGLEVPRYRRKRHISVRDMNALLDYH | 70 |
| Query: 70 | NKLRSQVYPTASNMEYMTWDVELERSAESRAESCLWEHGPASLLPSIGQNLGAHWGRYRP | 129 |
| | \| +\|+ \|\|\| \|+\|\|\|\|\| \|\| \| \| \|\| \| \|+\| \|\|\|+ \|+ +\|\|\|\| \| \|+\|\| | |
| Sbjct: 71 | NHIRASVYPPAANMEYMVWDKRLARAAEAWATQCIWAHGPSQLMRYVGQNLSIHSGQYRS | 130 |
| Query: 130 | PTFHVQSWYDEVKDFSYPYEHECNPYCPFRCSGPVCTHYTQVVWATSNRIGCAINLCHNM | 189 |
| | ++\|\| +\| + +\| +\|\|\|+\|\|+\|\| \|\| \|+\|\|\|\|+\|\|\|+\|\|\|+\|\|\|\|+ \| ++ | |
| Sbjct: 131 | VVDLMKSWSEEKWHYLFPAPRDCNPHCPWRCDGPTCSHYTQMVWASSNRLGCAIHTCSSI | 190 |
| Query: 190 | NIWGQIWPKAVYLVCNYSPKGNWWGHAPYKHGRPCSACPPSFGGGCRENLCYK | 242 |
| | ++\|\| \| +\| \|\|\|\|\|\|+ \|\|\|\| \| +\|\|\| \|+\|\|\|+\|\|\|\|+ \| \| \|+\|+\| | |
| Sbjct: 191 | SVWGNTWHRAAYLVCNYAIKGNWIGESPYKMGKPCSSCPPSYQGSCNSNMCFK | 243 |

The FCTR7 amino acid has 129 of 237 amino acid residues (54%) identical to, and 167 of 237 amino acid residues (70%) similar to, the 258 amino acid 25 kDa Trypsin Inhibitor from Homo sapiens (EMBLAcc No.: 043692) (SEQ ID NO:88) (Table 7J).

TABLE 7J
BLASTP alignments of FCTR7 against 25 kDa
Trypsin Inhibitor, (SEQ ID NO:88)

ptnr:SPTREMBL-ACC:O43692 25 KDA TRYPSIN INHIBITOR - Homo sapiens
(Human), 258 aa.
Score = 743 (261.5 bits), Expect = 1.6e-73, P = 1.6e-73
Identities = 129/237 (54%), Positives = 167/237 (70%)

The FCTR7 amino acid has 79 of 193 amino acid residues (40%) identical to, and 110 of 193 amino acid residues (56%) similar to, the 266 amino acid Glioma Pathogenesis-Related Protein (RTVP-1 Protein)—Homo sapiens (SWISSPROT Acc No.: P48060) (SEQ ID NO:90) (Table 7K).

TABLE 7K
BLASTP alignments of FCTR7 against Glioma Pathogenesis-
Related Protein, (SEQ ID NO:90)

ptnr:SWISSPROT-ACC:P48060 OLIOMA PATHOORNESIS-RELATED PROTEIN
(RTVP-1 PROTEIN) - Homo sapiens (Human), 266 aa
Score = 314 (110.5 bits), Expect = 4.7e-28, P = 4.7e-28
Identities = 79/193 (40%), Positives = 110/193 (56%)

The FCTR7 amino acid has 66 of 186 amino acid residues (35%) identical to, and 91 of 186 amino acid residues (48%) similar to, the 186 amino acid Neutrophil granules matrix glycoprotein SGP28 precursor from Homo sapiens (SWISSPROT Acc No.: S68691) (SEQ ID NO:98) (Table 7L).

TABLE 7L
BLASTP alignments of FCTR7 against Neutrophil granules
matrix glycoprotein, (SEQ ID NO:98)

ptnr:PIR-ID:S68691 neutrophil granules matrix glycoprotein
SGP28 precursor - Human
Score = 254 (89.4 bits), Expect = 1.1e-21, P = 1.1e-21
Identities = 66/186 (35%), Positives = 91/186 (48%)

A novel developmentally regulated gene with homology to a tumor derived trypsin inhibitor is expressed in lung mesenchyme, as described in Am. J. Physiol. 0:0-0(1999). cDNA cloning of a novel trypsin inhibitor with similarity to pathogenesis-related proteins, and its frequent expression in human brain cancer cells is disclosed in Biochim. Biophys. Acta 1395:202-208(1998). RTVP-1, a novel human gene with sequence similarity to genes of diverse species, is expressed in tumor cell lines of glial but not neuronal origin, as published in Gene 180:125-130(1996). The human glioma pathogenesis-related protein is structurally related to plan pathogenesis-related proteins and its gene is expressed specifically in brain tumors (Gene 159:131-135(1995)). Structure comparison of human glioma pathogenesis-related protein GliPR and the plant pathogenesis-related protein P14a indicates a functional link between the human immune system and a plant defense system (Proc. Natl. Acad. Sci. U.S.A. 95:2262-2266(1998)). GliPR is highly expressed in the human brain tumor, glioblastoma multiform/astrocytome, but neither in normal fetal or adult brain tissue, nor in other nervous system tumors. GliPR belongs to a family that groups mammalian SCP/TPX1; insects AG3/AG5; FUNGI SC7/SC14 and plants PR-1. SGP28, a novel matrix glycoprotein in specific granules of human neutrophils with similarity to a human testis-specific gene product and to a rodent sperm-coating glycoprotein (FEBS Lett. 380, 246-250, 1996). The primary structure and properties of helothermine, a peptide toxin that blocks ryanodine receptors is described in Biophys. J. 68:2280-2288(1995). As GliPR, Helothermine belongs to a family that groups mammalian SCP/TPX 1; insects AG3/AG5; FUNGI SC7/SC14 and plants PR-1.

Based upon homology, FCTR7 protein and each homologous protein or peptide may share at least some activity.

Therapeutic uses:

FCTR7 protein has homology to trypsin inhibitors, Q91055 helothermine, tumor derived tyrpsin inhibitors, glioma pathogenesis-related protein, Q9ZOU6 LATE GESTATION LUNG PROTEIN 1, and to the Prosite family which groups mammalian SCP/TPX1;INSECTS AG3/AG5; FUNGI SC7/SC14 AND PLANTS PR-1 proteins. Therefore the FCTR7 protein disclosed in this invention could function like the proteins which it has homology to. These functions include tissue development in vitro and in vivo, and cancer pathogenesis.

Based the tissue expression pattern, the gene is implicated in diseases of tissues in which it is expressed. These diseases include but are not limited to:

Glioma,

cancer,

lung diseases,

gestation,

male and female reproductive diseases,

deafness,

neurological disorders,

gastric disorders, and

pancreatic diseases like diabetes.

These materials are further useful in the generation of antibodies that bind immunospecifically to the novel FCTR7 substances for use in therapeutic or diagnostic methods. These antibodies may be generated according to methods known in the art, using prediction from hydrophobicity charts, as described in the “Anti-FCTRX Antibodies” section below. In one embodiment, a contemplated FCTR7 epitope is from aa 40 to 120. In another embodiment, a FCTR7 epitope is from aa 130 to 170. In additional embodiments, FCTR7 epitopes are from aa 210 to 230, and from aa 240 to 280.

TABLE 8A
Summary Of Nucleic Acids And Proteins Of The Invention

| | | | Nucleic Acid | Amino Acid |
|---|---|---|---|---|
| Name | Tables | Clone; Description of Homolog | SEQ ID NO | SEQ ID NO |
| FCTR1 | 1A, 1B, | 58092213.0.36 follistatin-like protein | 1 | 2 |
| FCTR2 | 2A, 2B | AC012614_1.0.123; KIAA1061-like protein | 3 | 4 |
| FCTR3 | 3A, 3B | 10129612.0.118; neurestin-like protein | 5 | 6 |
| | 3C, 3D | 10129612.0.405; neurestin-like protein | 7 | 8 |
| | 3E | 10129612.0.154; neurestin-like protein | 9 | |
| | 3F | 10129612.0.67; neurestin-like protein | 10 | |
| | 3G | 10129612.0.258; neurestin-like protein | 11 | |
| | 3H, 3I | 10129612.0.352; neurestin-like protein | 12 | 13 |
| FCTR4 | 4A, 4B | 29692275.0.1; NF-Kappa-B P65delta3-like | 14 | 15 |
| | | protein | | |
| FCTR5 | 5A, 5B | 32125243.0.21; human complement C1R | 16 | 17 |
| | | component precursor-like protein | | |
| | 5C, 5D | | 18 | 19 |
| FCTR6 | 6A, 6B | 27455183.0.19; novel human blood | 20 | 21 |
| | | coagulation factor XI-like protein | | |
| | 6C, 6D | 27455183.0.145; novel human blood | 22 | 23 |
| | | coagulation factor XI-like protein | | |
| FCTR7 | 7A, 7B | 32592466.0.64; trypsin inhibitor-like | 24 | 25 |
| | | protein | | |
| FCTR1 | Example 2 | Ag809 Forward | 26 | |
| FCTR1 | Example 2 | Ag809 Probe | 27 | |
| FCTR1 | Example 2 | Ag809 Reverse | 28 | |
| FCTR4 | Example 2 | Ag2773 Forward | 29 | |
| FCTR4 | Example 2 | Ag2773 Probe | 30 | |
| FCTR4 | Example 2 | Ag2773 Reverse | 31 | |
| FCTR5 | Example 2 | Ag427 Forward | 32 | |
| FCTR5 | Example 2 | Ag427 Probe | 33 | |
| FCTR5 | Example 2 | Ag427 Reverse | 34 | |
| FCTR6 | Example 2 | Ag1541 Forward | 35 | |
| FCTR6 | Example 2 | Ag1541 Probe | 36 | |
| FCTR6 | Example 2 | Ag1541 Reverse | 37 | |

TABLE 8B
Summary of Query Sequences Disclosed

| Table | Database | Acc. No. | Sequence Name | Species | SEQ ID NO. |
|---|---|---|---|---|---|
| 1C, 1K | remtrEmbl | BAA21725 | IGFBP-like protein | mouse | 38 |
| 1D | sptrEmbl | Q61581 | Follistatin-like protein-2 | Mouse | 39 |
| 1E | SptrEmbl | Q07822 | Mac25 protein | Human | 40 |
| 1F, 1K | SptrEmbl | O88812 | Mac25 protein | Mouse | 41 |
| 1G, 1K | SptrEmbl | Q16270 | Prostacyclin-stimulating factor | Human | 42 |
| 1H, 1K | PIR | B40098 | Colorectal cancer suppressor | Rat | 43 |
| 1I | TrEmblne | AAD9360 | PTP sigma (brain) precursor | Human | 44 |
| | w | | | | |
| 1J | SptrEmbl | Q13332 | PTP sigma precursor | Human | 45 |
| 2C | GenBank | AB028984 | KIAA1061 cDNA | Human | 46 |
| 2D | TrEmblne | BAA85677 | KIAA1263 | Human | 47 |
| | w | | | | |
| 2E | TrEmblne | BAA83013 | KIAA1061 protein fragment | Human | 48 |
| | w | | | | |
| 2F | Embl | CAB70877.1 | Hypothetical protein DKFzp566D234.1 | Human | 49 |
| 2G | GenBank | Q62632 | Follistatin-related protein-1 precursor | Rat | 50 |
| 2H | GenBank | Q62536 | Follistatin-related protein-1 precursor | Mouse | 51 |
| 2I | GenBank | JG0187 | Follistatin related protein | African | 52 |
| | | | | clawed | |
| | | | | frog | |
| 2J | GenBank | Q12841 | Follistatin related protein-1 precursor | Human | 53 |
| 2K | Embl | CAB42968.1 | Flik protein | Chicken | 54 |
| 2L | GenBank | T13822 | Frazzled gene protein | Fruit fly | 55 |
| 2M | GenBank | AAC38849.1 | Roundabout 1 | Fruit fly | 56 |
| 2N | GenBank | O60469 | Down Syndrome Cell Adhesion Molecule | Human | 57 |
| | | | Precursor | | |
| 2O | SwissProt | Q13449 | Limbic system-associated membrane | Human | 58 |
| | | | protein precursor | | |
| 2P | SptrEmbl | O70246 | Putative neuronal cell adhesion molecule, | Mouse | 59 |
| | | | short form | | |
| 2Q | SptrEmbl | O02869 | CHLAMP, G11-isoform precursor | Chicken | 60 |
| 2R | SwissProt | Q62813 | Limbic system-associated membrane | Rat | 61 |
| | | | protein precursor | | |
| 3J | GenBank | NM_011856.2 | Odd Oz/ten-m homology 2 | Fruit fly | 62 |
| 3K | Embl | AJ245711.1 | Teneurin-2 cDNA, short splice variant | Chicken | 63 |
| 3L | GenBank | AB032953 | KIAA 1127 cDNA | Human | 64 |
| 3M, 3U | GenBank | AB025411 | Ten-m2 cDNA | Mouse | 65 |
| 3N | GenBank | NM_020088.1 | Neurestin alpha cDNA | Rat | 66 |
| 3O | Embl | GGA278031 | Teneurin-2 | Chicken | 67 |
| 3P | GenBank | NP_035986.2 | Odd Oz/ten-m homology 2 | Fruit fly | 68 |
| 3Q | Embl | CAC09416.1 | Teneurin-2 | Chicken | 69 |
| 3R | GenBank | BAA77399.1 | Ten-m4 | Mouse | 70 |
| 3S | GenBank | AB032953 | KIAA1127 protein | Human | 71 |
| 3T | GenBank | AF086607 | Neurestin alpha | Rat | 72 |
| 4C | SptrEmbl | Q99233 | Hypothetical 10 kD protein | Trypanosome | 73 |
| 4C | SptrEmbl | Q16896 | GABA receptor subunit | | 74 |
| 4C | SptrEmbl | O76473 | GABA receptor subunit | | 75 |
| 4C | TrEmblne | AAD28317 | FI3J11.13 protein | | 76 |
| | w | | | | |
| Text p. 90 | SptrEmbl | Q13313 | NF-kappa B P65 delta 3 protein | Human | 77 |
| 5E | GenBank | XM_007061.1 | Complement C1R-like proteinase | Human | 78 |
| | | | precursor | | |
| 5F | GenBank | NM_001733.1 | Complement component 1, R | Human | 79 |
| | | | subcomponent cDNA | | |
| 5G | GenBank | AAF44349.1 | Complement C1R-like proteinase | Human | 80 |
| | | | precursor | | |
| 5H | GenBank | AAA5185.1 | Complement C1R component precursor | Human | 81 |
| 6E | GenBank | AB046651 | Brain cDNA clone Qcc-17034 | Macaque | 82 |
| 6F | GenBank | AK09660 | Adult testis cDNA, RIKEN full length | Mouse | 83 |
| | | | enriched | | |
| 6G | GenBank | AB046651 | Hypothetical protein | Macaque | 84 |
| 6H | GenBank | NP_000838.1 | Plasma kallikrein B1 precursor | Human | 85 |
| 6I | GenBank | BAA37147.1 | Kallikrein | Pig | 86 |
| 6J | Embl | CAA64368.1 | Coagulation factor XI | Human | 87 |
| 7D, 7J | SptrEmbl | O43692 | 25 kDa trypsin inhibitor | Human | 88 |
| 7D | SptrEmbl | O44228 | HRTT-1 | | 89 |
| 7D, 7K | SptrEmbl | P418060 | Glioma pathogenesis-related protein | Human | 90 |
| 7D | PIR-ID | JC4131 | Glioma pathogenesis-related protein | Human | 91 |
| 7D | SwissProt | O19010 | Cysteine-rcih secretory protein | | 92 |
| 7E | GenBank | AF142573 | Putatitive secretory protein precursor | Human | 93 |
| | | | cDNA | | |
| 7F | GenBank | AF142573 | Putative secretory protein precursor | Human | 94 |
| 7G | GenBank | AF109674 | Late gestation lung protein 1 | Rat | 95 |
| 7H | GenBank | D45027 | R3H domain containing preprotein, 25 | Human | 96 |
| | | | kDa trypsin inhibitor | | |
| 7I | Embl | AL117382 | Novel protein similar to a trypsin | Human | 97 |
| | | | inhibitor | | |
| 7L | PIR-ID | S68691 | Neutrophil granules matrix glycoprotein | Human | 98 |
| | | | SGP28 precursor | | |

FCTRX Nucleic Acids and Polypeptides

One aspect of the invention pertains to isolated nucleic acid molecules that encode FCTRX polypeptides or biologically-active portions thereof. Also included in the invention are nucleic acid fragments sufficient for use as hybridization probes to identify FCTRX-encoding nucleic acids (e.g., FCTRX mRNAs) and fragments for use as PCR primers for the amplification and/or mutation of FCTRX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

An FCTRX nucleic acid can encode a mature FCTRX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full length gene product, encoded by the corresponding gene. Alternatively, it may be defined as the polypeptide, precursor or proprotein encoded by an ORF described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

The term “probes”, as utilized herein, refers to nucleic acid sequences of variable length, preferably between at least about 10 nucleotides (nt), 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single- or double-stranded and designed to have specificity in PCR, membrane-based hybridization technologies, or ELISA-like technologies.

The term “isolated” nucleic acid molecule, as utilized herein, is one which is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated FCTRX nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the invention, e.g., a nucleic acid molecule having the nucleotide sequence of SEQ ID NOS:1, 3, 5, 7, 9, 10, 11, 12, 14, 16, 18, 20, 22, and 24, or a complement of this aforementioned nucleotide sequence, can be isolated using standard molecular biology techniques and the sequence information provided herein. Using all or a portion of the nucleic acid sequence of SEQ ID NOS: 1, 3, 5, 7, 9, 10, 11, 12, 14, 16, 18, 20, 22, and 24 as a hybridization probe, FCTRX molecules can be isolated using standard hybridization and cloning techniques (e.g., as described in Sambrook, et al., (eds.), MOLECULAR CLONING: A LABORATORY MANUAL 2^{nd} Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to FCTRX nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides of SEQ ID NOS: 1, 3, 5, 7, 9, 10, 11, 12, 14, 16, 18, 20, 22, and 24, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NOS: 1, 3, 5, 7, 9, 10, 11, 12, 14, 16, 18, 20, 22, and 24, or a portion of this nucleotide sequence (e.g., a fragment that can be used as a probe or primer or a fragment encoding a biologically-active portion of an FCTRX polypeptide). A nucleic acid molecule that is complementary to the nucleotide sequence shown in SEQ ID NOS:1, 3, 5, 7, 9, 10, 11, 12, 14, 16, 18, 20, 22, and 24, is one that is sufficiently complementary to the nucleotide sequence shown in SEQ ID NOS:1, 3, 5, 7, 9, 10, 11, 12, 14, 16, 18, 20, 22, and 24, that it can hydrogen bond with little or no mismatches to the nucleotide sequence shown in SEQ ID NOS:1, 3, 5, 7, 9, 10, 11, 12, 14, 16, 18, 20, 22, and 24, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of FCTRX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for an FCTRX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human FCTRX protein. Homologous nucleic acid sequences include those nucleic acid sequences that encode conservative amino acid substitutions (see below) in SEQ ID NOS:2, 4, 6, 8, 13, 15, 17, 19, 21, 23, and 25, as well as a polypeptide possessing FCTRX biological activity. Various biological activities of the FCTRX proteins are described below.

An FCTRX polypeptide is encoded by the open reading frame (“ORF”) of an FCTRX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bonafide cellular protein, a minimum size requirement is often set, e.g., a stretch of DNA that would encode a protein of 50 amino acids or more.

The nucleotide sequences determined from the cloning of the human FCTRX genes allows for the generation of probes and primers designed for use in identifying and/or cloning FCTRX homologues in other cell types, e.g. from other tissues, as well as FCTRX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence of SEQ ID NOS: 1, 3, 5, 7, 9, 10, 11, 12, 14, 16, 18, 20, 22, and 24; or an anti-sense strand nucleotide sequence of SEQ ID NOS:1, 3, 5, 7, 9, 10, 11, 12, 14, 16, 18, 20, 22, and 24; or of a naturally occurring mutant of SEQ ID NOS:1, 3, 5, 7, 9, 10, 11, 12, 14, 16, 18, 20, 22, and 24.

Probes based on the human FCTRX nucleotide sequences can be used to detect transcripts or genomic sequences encoding the same or homologous proteins. In various embodiments, the probe further comprises a label group attached thereto, e.g. the label group can be a radioisotope, a fluorescent compound, an enzyme, or an enzyme co-factor. Such probes can be used as a part of a diagnostic test kit for identifying cells or tissues which mis-express an FCTRX protein, such as by measuring a level of an FCTRX-encoding nucleic acid in a sample of cells from a subject e.g., detecting FCTRX mRNA levels or determining whether a genomic FCTRX gene has been mutated or deleted.

“A polypeptide having a biologically-active portion of an FCTRX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of FCTRX” can be prepared by isolating a portion of SEQ ID NOS:1, 3, 5, 7, 9, 10, 11, 12, 14, 16, 18, 20, 22, and 24, that encodes a polypeptide having an FCTRX biological activity (the biological activities of the FCTRX proteins are described below), expressing the encoded portion of FCTRX protein (e.g., by recombinant expression in vitro) and assessing the activity of the encoded portion of FCTRX.

FCTRX Nucleic Acid and Polypeptide Variants

The invention further encompasses nucleic acid molecules that differ from the nucleotide sequences shown in SEQ ID NOS:1, 3, 5, 7, 9, 10, 11, 12, 14, 16, 18, 20, 22, and 24, due to degeneracy of the genetic code and thus encode the same FCTRX proteins as that encoded by the nucleotide sequences shown in SEQ ID NO NOS: 1, 3, 5, 7, 9, 10, 11, 12, 14, 16, 18, 20, 22, and 24. In another embodiment, an isolated nucleic acid molecule of the invention has a nucleotide sequence encoding a protein having an amino acid sequence shown in SEQ ID NOS:2, 4, 6, 8, 13, 15, 17, 19, 21, 23, and 25.

In addition to the human FCTRX nucleotide sequences shown in SEQ ID NOS: 1, 3, 5, 7, 9, 10, 11, 12, 14, 16, 18, 20, 22, and 24, it will be appreciated by those skilled in the art that DNA sequence polymorphisms that lead to changes in the amino acid sequences of the FCTRX polypeptides may exist within a population (e.g., the human population). Such genetic polymorphism in the FCTRX genes may exist among individuals within a population due to natural allelic variation. As used herein, the terms “gene” and “recombinant gene” refer to nucleic acid molecules comprising an open reading frame (ORF) encoding an FCTRX protein, preferably a vertebrate FCTRX protein. Such natural allelic variations can typically result in 1-5% variance in the nucleotide sequence of the FCTRX genes. Any and all such nucleotide variations and resulting amino acid polymorphisms in the FCTRX polypeptides, which are the result of natural allelic variation and that do not alter the functional activity of the FCTRX polypeptides, are intended to be within the scope of the invention.

Moreover, nucleic acid molecules encoding FCTRX proteins from other species, and thus that have a nucleotide sequence that differs from the human sequence of SEQ ID NOS: 1, 3, 5, 7, 9, 10, 11, 12, 14, 16, 18, 20, 22, and 24, are intended to be within the scope of the invention. Nucleic acid molecules corresponding to natural allelic variants and homologues of the FCTRX cDNAs of the invention can be isolated based on their homology to the human FCTRX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NOS: 1, 3, 5, 7, 9, 10, 11, 12, 14, 16, 18, 20, 22, and 24. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding FCTRX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6× SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2× SSC, 0.01% BSA at 50° C. An isolated nucleic acid molecule of the invention that hybridizes under stringent conditions to the sequences of SEQ ID NOS:1, 3, 5, 7, 9, 10, 11, 12, 14, 16, 18, 20, 22, and 24, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NOS:1, 3, 5, 7, 9, 10, 11, 12, 14, 16, 18, 20, 22, and 24, or fragments, analogs or derivatives thereof, under conditions of moderate stringency is provided. A non-limiting example of moderate stringency hybridization conditions are hybridization in 6× SSC, 5× Denhardt's solution, 0.5% SDS and 100 mg/ml denatured salmon sperm DNA at 55° C., followed by one or more washes in 1× SSC, 0.1% SDS at 37° C. Other conditions of moderate stringency that may be used are well-known within the art. See, e.g., Ausubel, et al. (eds.), 1993, CURRENT PROTOCOLS IN MOLECULAR BIOLOGY, John Wiley & Sons, NY, and Kriegler, 1990; GENE TRANSFER AND EXPRESSION, A LABORATORY MANUAL, Stockton Press, NY.

In a third embodiment, a nucleic acid that is hybridizable to the nucleic acid molecule comprising the nucleotide sequences of SEQ ID NOS:1, 3, 5, 7, 9, 10, 11, 12, 14, 16, 18, 20, 22, and 24, or fragments, analogs or derivatives thereof, under conditions of low stringency, is provided. A non-limiting example of low stringency hybridization conditions are hybridization in 35% formamide, 5× SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2× SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981. Proc Natl Acad Sci USA 78: 6789-6792.

Conservative Mutations

In addition to naturally-occurring allelic variants of FCTRX sequences that may exist in the population, the skilled artisan will further appreciate that changes can be introduced by mutation into the nucleotide sequences of SEQ ID NO NOS:1, 3, 5, 7, 9, 10, 11, 12, 14, 16, 18, 20, 22, and 24, thereby leading to changes in the amino acid sequences of the encoded FCTRX proteins, without altering the functional ability of said FCTRX proteins. For example, nucleotide substitutions leading to amino acid substitutions at “non-essential” amino acid residues can be made in the sequence of SEQ ID NOS:2, 4, 6, 8, 13, 15, 17, 19, 21, 23, and 25. A “non-essential” amino acid residue is a residue that can be altered from the wild-type sequences of the FCTRX proteins without altering their biological activity, whereas an “essential” amino acid residue is required for such biological activity. For example, amino acid residues that are conserved among the FCTRX proteins of the invention are predicted to be particularly non-amenable to alteration. Amino acids for which conservative substitutions can be made are well-known within the art.

Another aspect of the invention pertains to nucleic acid molecules encoding FCTRX proteins that contain changes in amino acid residues that are not essential for activity. Such FCTRX proteins differ in amino acid sequence from SEQ ID NOS:2, 4, 6, 8, 13, 15, 17, 19, 21, 23, and 25, yet retain biological activity. In one embodiment, the isolated nucleic acid molecule comprises a nucleotide sequence encoding a protein, wherein the protein comprises an amino acid sequence at least about 45% homologous to the amino acid sequences of SEQ ID NOS:2, 4, 6, 8, 13, 15, 17, 19, 21, 23, and 25. Preferably, the protein encoded by the nucleic acid molecule is at least about 60% homologous to SEQ ID NOS:2, 4, 6, 8, 13, 15, 17, 19, 21, 23, and 25; more preferably at least about 70% homologous to SEQ ID NOS:2, 4, 6, 8, 13, 15, 17, 19, 21, 23, and 25; still more preferably at least about 80% homologous to SEQ ID NOS:2, 4, 6, 8, 13, 15, 17, 19, 21, 23, and 25; even more preferably at least about 90% homologous to SEQ ID NOS:2, 4, 6, 8, 13, 15, 17, 19, 21, 23, and 25; and most preferably at least about 95% homologous to SEQ ID NOS:2, 4, 6, 8, 13, 15, 17, 19, 21, 23, and 25.

An isolated nucleic acid molecule encoding an FCTRX protein homologous to the protein of SEQ ID NOS:2, 4, 6, 8, 13, 15, 17, 19, 21, 23, and 25, can be created by introducing one or more nucleotide substitutions, additions or deletions into the nucleotide sequence of SEQ ID NOS:1, 3, 5, 7, 9, 10, 11, 12, 14, 16, 18, 20, 22, and 24, such that one or more amino acid substitutions, additions or deletions are introduced into the encoded protein.

Mutations can be introduced into SEQ ID NOS:2, 4, 6, 8, 13, 15, 17, 19, 21, 23, and 25, by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the FCTRX protein is replaced with another amino acid residue from the same side chain family. Alternatively, in another embodiment, mutations can be introduced randomly along all or part of an FCTRX coding sequence, such as by saturation mutagenesis, and the resultant mutants can be screened for FCTRX biological activity to identify mutants that retain activity. Following mutagenesis of SEQ ID NOS:2, 4, 6, 8, 13, 15, 17, 19, 21, 23, and 25, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, VLIM, HFY, wherein the letters within each group represent the single letter amino acid code.

In one embodiment, a mutant FCTRX protein can be assayed for (i) the ability to form protein:protein interactions with other FCTRX proteins, other cell-surface proteins, or biologically-active portions thereof, (ii) complex formation between a mutant FCTRX protein and an FCTRX ligand; or (iii) the ability of a mutant FCTRX protein to bind to an intracellular target protein or biologically-active portion thereof; (e.g. avidin proteins).

In yet another embodiment, a mutant FCTRX protein can be assayed for the ability to regulate a specific biological function (e.g., regulation of insulin release).

Antisense Nucleic Acids

Another aspect of the invention pertains to isolated antisense nucleic acid molecules that are hybridizable to or complementary to the nucleic acid molecule comprising the nucleotide sequence of SEQ ID NOS:1, 3, 5, 7, 9, 10, 11, 12, 14, 16, 18, 20, 22, and 24, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire FCTRX coding strand, or to only a portion thereof. Nucleic acid molecules encoding fragments, homologs, derivatives and analogs of an FCTRX protein of SEQ ID NOS:2, 4, 6, 8, 13, 15, 17, 19, 21, 23, and 25; or antisense nucleic acids complementary to an FCTRX nucleic acid sequence of SEQ ID NOS:1, 3, 5, 7, 9, 10, 11, 12, 14, 16, 18, 20, 22, and 24, are additionally provided.

In one embodiment, an antisense nucleic acid molecule is antisense to a “coding region” of the coding strand of a nucleotide sequence encoding an FCTRX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the FCTRX protein. The term “noncoding region” refers to 5′ and 3′ sequences which flank the coding region that are not translated into amino acids (i.e., also referred to as 5′ and 3′ untranslated regions).

Given the coding strand sequences encoding the FCTRX protein disclosed herein, antisense nucleic acids of the invention can be designed according to the rules of Watson and Crick or Hoogsteen base pairing. The antisense nucleic acid molecule can be complementary to the entire coding region of FCTRX mRNA, but more preferably is an oligonucleotide that is antisense to only a portion of the coding or noncoding region of FCTRX mRNA. For example, the antisense oligonucleotide can be complementary to the region surrounding the translation start site of FCTRX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an FCTRX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other. See, e.g., Gaultier, et al., 1987. Nucl. Acids Res. 15: 6625-6641. The antisense nucleic acid molecule can also comprise a 2′-o-methylribonucleotide (see, e.g., Inoue, et al. 1987. Nucl. Acids Res. 15: 6131-6148) or a chimeric RNA-DNA analogue (see, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave FCTRX mRNA transcripts to thereby inhibit translation of FCTRX mRNA. A ribozyme having specificity for an FCTRX-encoding nucleic acid can be designed based upon the nucleotide sequence of an FCTRX cDNA disclosed herein (i.e., SEQ ID NOS: 1, 3, 5, 7, 9, 10, 11, 12, 14, 16, 18, 20, 22, and 24). For example, a derivative of a Tetrahymena L-19 IVS RNA can be constructed in which the nucleotide sequence of the active site is complementary to the nucleotide sequence to be cleaved in an FCTRX-encoding mRNA. See, e.g., U.S. Pat. No. 4,987,071 to Cech, et al. and U.S. Pat. No. 5,116,742 to Cech, et al. FCTRX mRNA can also be used to select a catalytic RNA having a specific ribonuclease activity from a pool of RNA molecules. See, e.g., Bartel et al., (1993) Science 261:1411-1418.

Alternatively, FCTRX gene expression can be inhibited by targeting nucleotide sequences complementary to the regulatory region of the FCTRX nucleic acid (e.g., the FCTRX promoter and/or enhancers) to form triple helical structures that prevent transcription of the FCTRX gene in target cells. See, e.g., Helene, 1991. Anticancer Drug Des. 6: 569-84; Helene, et al. 1992. Ann. N.Y. Acad. Sci. 660: 27-36; Maher, 1992. Bioassays 14: 807-15.

In various embodiments, the FCTRX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of FCTRX can be used in therapeutic and diagnostic applications. For example, PNAs can be used as antisense or antigene agents for sequence-specific modulation of gene expression by, e.g., inducing transcription or translation arrest or inhibiting replication. PNAs of FCTRX can also be used, for example, in the analysis of single base pair mutations in a gene (e.g., PNA directed PCR clamping; as artificial restriction enzymes when used in combination with other enzymes, e.g., SI nucleases (see, Hyrup, et al., 1996.supra); or as probes or primers for DNA sequence and hybridization (see, Hyrup, et al., 1996, supra; Perry-O'Keefe, et al., 1996. supra).

In another embodiment, PNAs of FCTRX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of FCTRX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleobases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

FCTRX Polypeptides

A polypeptide according to the invention includes a polypeptide including the amino acid sequence of FCTRX polypeptides whose sequences are provided in SEQ ID NOS:2, 4, 6, 8, 13, 15, 17, 19, 21, 23, and 25. The invention also includes a mutant or variant protein any of whose residues may be changed from the corresponding residues shown in SEQ ID NOS:2, 4, 6, 8, 13, 15, 17, 19, 21, 23, and 25, while still encoding a protein that maintains its FCTRX activities and physiological functions, or a functional fragment thereof.

In general, an FCTRX variant that preserves FCTRX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated FCTRX proteins, and biologically-active portions thereof, or derivatives, fragments, analogs or homologs thereof. Also provided are polypeptide fragments suitable for use as immunogens to raise anti-FCTRX antibodies. In one embodiment, native FCTRX proteins can be isolated from cells or tissue sources by an appropriate purification scheme using standard protein purification techniques. In another embodiment, FCTRX proteins are produced by recombinant DNA techniques. Alternative to recombinant expression, an FCTRX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the FCTRX protein is derived, or substantially free from chemical precursors or other chemicals when chemically synthesized. The language “substantially free of cellular material” includes preparations of FCTRX proteins in which the protein is separated from cellular components of the cells from which it is isolated or recombinantly-produced. In one embodiment, the language “substantially free of cellular material” includes preparations of FCTRX proteins having less than about 30% (by dry weight) of non-FCTRX proteins (also referred to herein as a “contaminating protein”), more preferably less than about 20% of non-FCTRX proteins, still more preferably less than about 10% of non-FCTRX proteins, and most preferably less than about 5% of non-FCTRX proteins. When the FCTRX protein or biologically-active portion thereof is recombinantly-produced, it is also preferably substantially free of culture medium, i.e., culture medium represents less than about 20%, more preferably less than about 10%, and most preferably less than about 5% of the volume of the FCTRX protein preparation.

The language “substantially free of chemical precursors or other chemicals” includes preparations of FCTRX proteins in which the protein is separated from chemical precursors or other chemicals that are involved in the synthesis of the protein. In one embodiment, the language “substantially free of chemical precursors or other chemicals” includes preparations of FCTRX proteins having less than about 30% (by dry weight) of chemical precursors or non-FCTRX chemicals, more preferably less than about 20% chemical precursors or non-FCTRX chemicals, still more preferably less than about 10% chemical precursors or non-FCTRX chemicals, and most preferably less than about 5% chemical precursors or non-FCTRX chemicals.

Biologically-active portions of FCTRX proteins include peptides comprising amino acid sequences sufficiently homologous to or derived from the amino acid sequences of the FCTRX proteins (e.g., the amino acid sequence shown in SEQ ID NOS:2, 4, 6, 8, 13, 15, 17, 19, 21, 23, and 25) that include fewer amino acids than the full-length FCTRX proteins, and exhibit at least one activity of an FCTRX protein. Typically, biologically-active portions comprise a domain or motif with at least one activity of the FCTRX protein. A biologically-active portion of an FCTRX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native FCTRX protein.

In an embodiment, the FCTRX protein has an amino acid sequence shown in SEQ ID NOS:2, 4, 6, 8, 13, 15, 17, 19, 21, 23, and 25. In other embodiments, the FCTRX protein is substantially homologous to SEQ ID NOS:2, 4, 6, 8, 13, 15, 17, 19, 21, 23, and 25, and retains the functional activity of the protein of SEQ ID NOS:2, 4, 6, 8, 13, 15, 17, 19, 21, 23, and 25, yet differs in amino acid sequence due to natural allelic variation or mutagenesis, as described in detail, below. Accordingly, in another embodiment, the FCTRX protein is a protein that comprises an amino acid sequence at least about 45% homologous to the amino acid sequence of SEQ ID NOS:2, 4, 6, 8, 13, 15, 17, 19, 21, 23, and 25, and retains the functional activity of the FCTRX proteins of SEQ ID NOS:2, 4, 6, 8, 13, 15, 17, 19, 21, 23, and 25.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NOS: 1, 3, 5, 7, 9, 10, 11, 12, 14, 16, 18, 20, 22, and 24.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides FCTRX chimeric or fusion proteins. As used herein, an FCTRX “chimeric protein” or “fusion protein” comprises an FCTRX polypeptide operatively-linked to a non-FCTRX polypeptide. An “FCTRX polypeptide” refers to a polypeptide having an amino acid sequence corresponding to an FCTRX protein (SEQ ID NOS:2, 4, 6, 8, 13, 15, 17, 19, 21, 23, and 25), whereas a “non-FCTRX polypeptide” refers to a polypeptide having an amino acid sequence corresponding to a protein that is not substantially homologous to the FCTRX protein, e.g., a protein that is different from the FCTRX protein and that is derived from the same or a different organism. Within an FCTRX fusion protein the FCTRX polypeptide can correspond to all or a portion of an FCTRX protein. In one embodiment, an FCTRX fusion protein comprises at least one biologically-active portion of an FCTRX protein. In another embodiment, an FCTRX fusion protein comprises at least two biologically-active portions of an FCTRX protein. In yet another embodiment, an FCTRX fusion protein comprises at least three biologically-active portions of an FCTRX protein. Within the fusion protein, the term “operatively-linked” is intended to indicate that the FCTRX polypeptide and the non-FCTRX polypeptide are fused in-frame with one another. The non-FCTRX polypeptide can be fused to the N-terminus or C-terminus of the FCTRX polypeptide.

In one embodiment, the fusion protein is a GST-FCTRX fusion protein in which the FCTRX sequences are fused to the C-terminus of the GST (glutathione S-transferase) sequences. Such fusion proteins can facilitate the purification of recombinant FCTRX polypeptides.

In another embodiment, the fusion protein is an FCTRX protein containing a heterologous signal sequence at its N-terminus. In certain host cells (e.g., mammalian host cells), expression and/or secretion of FCTRX can be increased through use of a heterologous signal sequence.

In yet another embodiment, the fusion protein is an FCTRX-immunoglobulin fusion protein in which the FCTRX sequences are fused to sequences derived from a member of the immunoglobulin protein family. The FCTRX-immunoglobulin fusion proteins of the invention can be incorporated into pharmaceutical compositions and administered to a subject to inhibit an interaction between an FCTRX ligand and an FCTRX protein on the surface of a cell, to thereby suppress FCTRX-mediated signal transduction in vivo. The FCTRX-immunoglobulin fusion proteins can be used to affect the bioavailability of an FCTRX cognate ligand. Inhibition of the FCTRX ligand/FCTRX interaction may be useful therapeutically for both the treatment of proliferative and differentiative disorders, as well as modulating (e.g. promoting or inhibiting) cell survival. Moreover, the FCTRX-immunoglobulin fusion proteins of the invention can be used as immunogens to produce anti-FCTRX antibodies in a subject, to purify FCTRX ligands, and in screening assays to identify molecules that inhibit the interaction of FCTRX with an FCTRX ligand.

An FCTRX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). An FCTRX-encoding nucleic acid can be cloned into such an expression vector such that the fusion moiety is linked in-frame to the FCTRX protein.

FCTRX Agonists and Antagonists

The invention also pertains to variants of the FCTRX proteins that function as either FCTRX agonists (i.e., mimetics) or as FCTRX antagonists. Variants of the FCTRX protein can be generated by mutagenesis (e.g., discrete point mutation or truncation of the FCTRX protein). An agonist of the FCTRX protein can retain substantially the same, or a subset of, the biological activities of the naturally occurring form of the FCTRX protein. An antagonist of the FCTRX protein can inhibit one or more of the activities of the naturally occurring form of the FCTRX protein by, for example, competitively binding to a downstream or upstream member of a cellular signaling cascade which includes the FCTRX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the FCTRX proteins.

Variants of the FCTRX proteins that function as either FCTRX agonists (i.e., mimetics) or as FCTRX antagonists can be identified by screening combinatorial libraries of mutants (e.g., truncation mutants) of the FCTRX proteins for FCTRX protein agonist or antagonist activity. In one embodiment, a variegated library of FCTRX variants is generated by combinatorial mutagenesis at the nucleic acid level and is encoded by a variegated gene library. A variegated library of FCTRX variants can be produced by, for example, enzymatically ligating a mixture of synthetic oligonucleotides into gene sequences such that a degenerate set of potential FCTRX sequences is expressible as individual polypeptides, or alternatively, as a set of larger fusion proteins (e.g., for phage display) containing the set of FCTRX sequences therein. There are a variety of methods which can be used to produce libraries of potential FCTRX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential FCTRX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

Polypeptide Libraries

In addition, libraries of fragments of the FCTRX protein coding sequences can be used to generate a variegated population of FCTRX fragments for screening and subsequent selection of variants of an FCTRX protein. In one embodiment, a library of coding sequence fragments can be generated by treating a double stranded PCR fragment of an FCTRX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with SI nuclease, and ligating the resulting fragment library into an expression vector. By this method, expression libraries can be derived which encodes N-terminal and internal fragments of various sizes of the FCTRX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of FCTRX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify FCTRX variants. See, e.g., Arkin and Yourvan, 1992. Proc. Natl. Acad. Sci. USA 89: 7811-7815; Delgrave, et al., 1993. Protein Engineering 6:327-331.

Anti-FCTRX Antibodies

The invention encompasses antibodies and antibody fragments, such as Fab or (Fab)2, that bind immunospecifically to any of the FCTRX polypeptides of said invention.

An isolated FCTRX protein, or a portion or fragment thereof, can be used as an immunogen to generate antibodies that bind to FCTRX polypeptides using standard techniques for polyclonal and monoclonal antibody preparation. The full-length FCTRX proteins can be used or, alternatively, the invention provides antigenic peptide fragments of FCTRX proteins for use as immunogens. The antigenic FCTRX peptides comprises at least 4 amino acid residues of the amino acid sequence shown in SEQ ID NO NOS:2, 4, 6, 8, 13, 15, 17, 19, 21, 23, and 25, and encompasses an epitope of FCTRX such that an antibody raised against the peptide forms a specific immune complex with FCTRX. Preferably, the antigenic peptide comprises at least 6, 8, 10, 15, 20, or 30 amino acid residues. Longer antigenic peptides are sometimes preferable over shorter antigenic peptides, depending on use and according to methods well known to someone skilled in the art.

In certain embodiments of the invention, at least one epitope encompassed by the antigenic peptide is a region of FCTRX that is located on the surface of the protein (e.g., a hydrophilic region). As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation (see, e.g., Hopp and Woods, 1981. Proc. Nat. Acad. Sci. USA 78: 3824-3828; Kyte and Doolittle, 1982. J. Mol. Biol. 157: 105-142, each incorporated herein by reference in their entirety).

As disclosed herein, FCTRX protein sequences of SEQ ID NOS:2, 4, 6, 8, 13, 15, 17, 19, 21, 23, and 25, or derivatives, fragments, analogs or homologs thereof, may be utilized as immunogens in the generation of antibodies that immunospecifically-bind these protein components. The term “antibody” as used herein refers to immunoglobulin molecules and immunologically-active portions of immunoglobulin molecules, i.e., molecules that contain an antigen binding site that specifically-binds (immunoreacts with) an antigen, such as FCTRX. Such antibodies include, but are not limited to, polyclonal, monoclonal, chimeric, single chain, F_{ab} and F_{(ab′)2} fragments, and an F_{ab} expression library. In a specific embodiment, antibodies to human FCTRX proteins are disclosed. Various procedures known within the art may be used for the production of polyclonal or monoclonal antibodies to an FCTRX protein sequence of SEQ ID NOS:2, 4, 6, 8, 13, 15, 17, 19, 21, 23, and 25, or a derivative, fragment, analog or homolog thereof. Some of these proteins are discussed below.

For the production of polyclonal antibodies, various suitable host animals (e.g., rabbit, goat, mouse or other mammal) may be immunized by injection with the native protein, or a synthetic variant thereof, or a derivative of the foregoing. An appropriate immunogenic preparation can contain, for example, recombinantly-expressed FCTRX protein or a chemically-synthesized FCTRX polypeptide. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), human adjuvants such as Bacille Calmette-Guerin and Corynebacterium parvum, or similar immunostimulatory agents. If desired, the antibody molecules directed against FCTRX can be isolated from the mammal (e.g., from the blood) and further purified by well known techniques, such as protein A chromatography to obtain the IgG fraction.

The term “monoclonal antibody” or “monoclonal antibody composition”, as used herein, refers to a population of antibody molecules that contain only one species of an antigen binding site capable of immunoreacting with a particular epitope of FCTRX. A monoclonal antibody composition thus typically displays a single binding affinity for a particular FCTRX protein with which it immunoreacts. For preparation of monoclonal antibodies directed towards a particular FCTRX protein, or derivatives, fragments, analogs or homologs thereof, any technique that provides for the production of antibody molecules by continuous cell line culture may be utilized. Such techniques include, but are not limited to, the hybridoma technique (see, e.g., Kohler & Milstein, 1975. Nature 256: 495-497); the trioma technique; the human B-cell hybridoma technique (see, e.g., Kozbor, et al., 1983. Immunol. Today 4: 72) and the EBV hybridoma technique to produce human monoclonal antibodies (see, e.g., Cole, et al., 1985. In: MONOCLONAL ANTIBODIES AND CANCER THERAPY, Alan R. Liss, Inc., pp. 77-96). Human monoclonal antibodies may be utilized in the practice of the invention and may be produced by using human hybridomas (see, e.g., Cote, et al., 1983. Proc Natl Acad Sci USA 80: 2026-2030) or by transforming human B-cells with Epstein Barr Virus in vitro (see, e.g., Cole, et al., 1985. In: MONOCLONAL ANTIBODIES AND CANCER THERAPY, Alan R. Liss, Inc., pp. 77-96). Each of the above citations is incorporated herein by reference in their entirety.

According to the invention, techniques can be adapted for the production of single-chain antibodies specific to an FCTRX protein (see, e.g., U.S. Pat. No. 4,946,778). In addition, methods can be adapted for the construction of Fab expression libraries (see, e.g., Huse, et al., 1989. Science 246: 1275-1281) to allow rapid and effective identification of monoclonal F_{ab} fragments with the desired specificity for an FCTRX protein or derivatives, fragments, analogs or homologs thereof. Non-human antibodies can be “humanized” by techniques well known in the art. See, e.g., U.S. Pat. No. 5,225,539. Antibody fragments that contain the idiotypes to an FCTRX protein may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent; and (iv) Fᵥ fragments.

Additionally, recombinant anti-FCTRX antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in International Application No. PCT/US86/02269; European Patent Application No. 184,187; European Patent Application No. 171,496; European Patent Application No. 173,494; PCT International Publication No. WO 86/01533; U.S. Pat. No. 4,816,567; U.S. Pat. No. 5,225,539; European Patent Application No. 125,023; Better, et al., 1988. Science 240: 1041-1043; Liu, et al., 1987. Proc. Natl. Acad. Sci. USA 84: 3439-3443; Liu, et al., 1987. J. Immunol. 139: 3521-3526; Sun, et al., 1987. Proc. Natl. Acad. Sci. USA 84: 214-218; Nishimura, et al., 1987. Cancer Res. 47: 999-1005; Wood, et al., 1985. Nature 314 :446-449; Shaw, et al., 1988. J. Natl. Cancer Inst. 80: 1553-1559); Morrison(1985) Science 229:1202-1207; Oi, et al. (1986) BioTechniques 4:214; Jones, et al., 1986. Nature 321: 552-525; Verhoeyan, et al., 1988. Science 239: 1534; and Beidler, et al., 1988. J. Immunol. 141: 4053-4060. Each of the above citations are incorporated herein by reference in their entirety.

In one embodiment, methods for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of an FCTRX protein is facilitated by generation of hybridomas that bind to the fragment of an FCTRX protein possessing such a domain. Thus, antibodies that are specific for a desired domain within an FCTRX protein, or derivatives, fragments, analogs or homologs thereof, are also provided herein.

Anti-FCTRX antibodies may be used in methods known within the art relating to the localization and/or quantitation of an FCTRX protein (e.g., for use in measuring levels of the FCTRX protein within appropriate physiological samples, for use in diagnostic methods, for use in imaging the protein, and the like). In a given embodiment, antibodies for FCTRX proteins, or derivatives, fragments, analogs or homologs thereof, that contain the antibody derived binding domain, are utilized as pharmacologically-active compounds (hereinafter “Therapeutics”).

An anti-FCTRX antibody (e.g., monoclonal antibody) can be used to isolate an FCTRX polypeptide by standard techniques, such as affinity chromatography or immunoprecipitation. An anti-FCTRX antibody can facilitate the purification of natural FCTRX polypeptide from cells and of recombinantly-produced FCTRX polypeptide expressed in host cells. Moreover, an anti-FCTRX antibody can be used to detect FCTRX protein (e.g., in a cellular lysate or cell supernatant) in order to evaluate the abundance and pattern of expression of the FCTRX protein. Anti-FCTRX antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

FCTRX Recombinant Expression Vectors and Host Cells

Another aspect of the invention pertains to vectors, preferably expression vectors, containing a nucleic acid encoding an FCTRX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., FCTRX proteins, mutant forms of FCTRX proteins, fusion proteins, etc.).

The recombinant expression vectors of the invention can be designed for expression of FCTRX proteins in prokaryotic or eukaryotic cells. For example, FCTRX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 3140), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the FCTRX expression vector is a yeast expression vector. Examples of vectors for expression in yeast Saccharomyces cerivisae include pYepSec1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, FCTRX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the (x-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to FCTRX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews—Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, FCTRX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding FCTRX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) FCTRX protein. Accordingly, the invention further provides methods for producing FCTRX protein using the host cells of the invention. In one embodiment, the method comprises culturing the host cell of invention (into which a recombinant expression vector encoding FCTRX protein has been introduced) in a suitable medium such that FCTRX protein is produced. In another embodiment, the method further comprises isolating FCTRX protein from the medium or the host cell.

Transgenic FCTRX Animals

The host cells of the invention can also be used to produce non-human transgenic animals. For example, in one embodiment, a host cell of the invention is a fertilized oocyte or an embryonic stem cell into which FCTRX protein-coding sequences have been introduced. Such host cells can then be used to create non-human transgenic animals in which exogenous FCTRX sequences have been introduced into their genome or homologous recombinant animals in which endogenous FCTRX sequences have been altered. Such animals are useful for studying the function and/or activity of FCTRX protein and for identifying and/or evaluating modulators of FCTRX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous FCTRX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing FCTRX-encoding nucleic acid into the male pronuclei of a fertilized oocyte (e.g., by microinjection, retroviral infection) and allowing the oocyte to develop in a pseudopregnant female foster animal. The human FCTRX cDNA sequences of SEQ ID NOS:1, 3, 5, 7, 9, 10, 11, 12, 14, 16, 18, 20, 22, and 24, can be introduced as a transgene into the genome of a non-human animal. Alternatively, a non-human homologue of the human FCTRX gene, such as a mouse FCTRX gene, can be isolated based on hybridization to the human FCTRX cDNA (described further supra) and used as a transgene. Intronic sequences and polyadenylation signals can also be included in the transgene to increase the efficiency of expression of the transgene. A tissue-specific regulatory sequence(s) can be operably-linked to the FCTRX transgene to direct expression of FCTRX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the FCTRX transgene in its genome and/or expression of FCTRX mRNA in tissues or cells of the animals. A transgenic founder animal can then be used to breed additional animals carrying the transgene. Moreover, transgenic animals carrying a transgene-encoding FCTRX protein can further be bred to other transgenic animals carrying other transgenes.

To create a homologous recombinant animal, a vector is prepared which contains at least a portion of an FCTRX gene into which a deletion, addition or substitution has been introduced to thereby alter, e.g., functionally disrupt, the FCTRX gene. The FCTRX gene can be a human gene (e.g., the cDNA of SEQ ID NOS: 1, 3, 5, 7, 9, 10, 11, 12, 14, 16, 18, 20, 22, and 24), but more preferably, is a non-human homologue of a human FCTRX gene. For example, a mouse homologue of human FCTRX gene of SEQ ID NOS: 1, 3, 5, 7, 9, 10, 11, 12, 14, 16, 18, 20, 22, and 24, can be used to construct a homologous recombination vector suitable for altering an endogenous FCTRX gene in the mouse genome. In one embodiment, the vector is designed such that, upon homologous recombination, the endogenous FCTRX gene is functionally disrupted (i.e., no longer encodes a functional protein; also referred to as a “knock out” vector).

Alternatively, the vector can be designed such that, upon homologous recombination, the endogenous FCTRX gene is mutated or otherwise altered but still encodes functional protein (e.g., the upstream regulatory region can be altered to thereby alter the expression of the endogenous FCTRX protein). In the homologous recombination vector, the altered portion of the FCTRX gene is flanked at its 5′- and 3′-termini by additional nucleic acid of the FCTRX gene to allow for homologous recombination to occur between the exogenous FCTRX gene carried by the vector and an endogenous FCTRX gene in an embryonic stem cell. The additional flanking FCTRX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced FCTRX gene has homologously-recombined with the endogenous FCTRX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The FCTRX nucleic acid molecules, FCTRX proteins, and anti-FCTRX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an FCTRX protein or anti-FCTRX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No.4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No.5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express FCTRX protein (e.g., via a recombinant expression vector in a host cell in gene therapy applications), to detect FCTRX mRNA (e.g., in a biological sample) or a genetic lesion in an FCTRX gene, and to modulate FCTRX activity, as described further, below. In addition, the FCTRX proteins can be used to screen drugs or compounds that modulate the FCTRX protein activity or expression as well as to treat disorders characterized by insufficient or excessive production of FCTRX protein or production of FCTRX protein forms that have decreased or aberrant activity compared to FCTRX wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease(possesses anti-microbial activity) and the various dyslipidemias. In addition, the anti-FCTRX antibodies of the invention can be used to detect and isolate FCTRX proteins and modulate FCTRX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to FCTRX proteins or have a stimulatory or inhibitory effect on, e.g., FCTRX protein expression or FCTRX protein activity. The invention also includes compounds identified in the screening assays described herein.

In one embodiment, the invention provides assays for screening candidate or test compounds which bind to or modulate the activity of the membrane-bound form of an FCTRX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37:1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of FCTRX protein, or a biologically-active portion thereof, on the cell surface is contacted with a test compound and the ability of the test compound to bind to an FCTRX protein determined. The cell, for example, can of mammalian origin or a yeast cell. Determining the ability of the test compound to bind to the FCTRX protein can be accomplished, for example, by coupling the test compound with a radioisotope or enzymatic label such that binding of the test compound to the FCTRX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of FCTRX protein, or a biologically-active portion thereof, on the cell surface with a known compound which binds FCTRX to form an assay mixture, contacting the assay mixture with a test compound, and determining the ability of the test compound to interact with an FCTRX protein, wherein determining the ability of the test compound to interact with an FCTRX protein comprises determining the ability of the test compound to preferentially bind to FCTRX protein or a biologically-active portion thereof as compared to the known compound.

In another embodiment, an assay is a cell-based assay comprising contacting a cell expressing a membrane-bound form of FCTRX protein, or a biologically-active portion thereof, on the cell surface with a test compound and determining the ability of the test compound to modulate (e.g., stimulate or inhibit) the activity of the FCTRX protein or biologically-active portion thereof. Determining the ability of the test compound to modulate the activity of FCTRX or a biologically-active portion thereof can be accomplished, for example, by determining the ability of the FCTRX protein to bind to or interact with an FCTRX target molecule. As used herein, a “target molecule” is a molecule with which an FCTRX protein binds or interacts in nature, for example, a molecule on the surface of a cell which expresses an FCTRX interacting protein, a molecule on the surface of a second cell, a molecule in the extracellular milieu, a molecule associated with the internal surface of a cell membrane or a cytoplasmic molecule. An FCTRX target molecule can be a non-FCTRX molecule or an FCTRX protein or polypeptide of the invention. In one embodiment, an FCTRX target molecule is a component of a signal transduction pathway that facilitates transduction of an extracellular signal (e.g. a signal generated by binding of a compound to a membrane-bound FCTRX molecule) through the cell membrane and into the cell. The target, for example, can be a second intercellular protein that has catalytic activity or a protein that facilitates the association of downstream signaling molecules with FCTRX.

Determining the ability of the FCTRX protein to bind to or interact with an FCTRX target molecule can be accomplished by one of the methods described above for determining direct binding. In one embodiment, determining the ability of the FCTRX protein to bind to or interact with an FCTRX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising an FCTRX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting an FCTRX protein or biologically-active portion thereof with a test compound and determining the ability of the test compound to bind to the FCTRX protein or biologically-active portion thereof. Binding of the test compound to the FCTRX protein can be determined either directly or indirectly as described above. In one such embodiment, the assay comprises contacting the FCTRX protein or biologically-active portion thereof with a known compound which binds FCTRX to form an assay mixture, contacting the assay mixture with a test compound, and determining the ability of the test compound to interact with an FCTRX protein, wherein determining the ability of the test compound to interact with an FCTRX protein comprises determining the ability of the test compound to preferentially bind to FCTRX or biologically-active portion thereof as compared to the known compound.

In still another embodiment, an assay is a cell-free assay comprising contacting FCTRX protein or biologically-active portion thereof with a test compound and determining the ability of the test compound to modulate (e.g. stimulate or inhibit) the activity of the FCTRX protein or biologically-active portion thereof. Determining the ability of the test compound to modulate the activity of FCTRX can be accomplished, for example, by determining the ability of the FCTRX protein to bind to an FCTRX target molecule by one of the methods described above for determining direct binding. In an alternative embodiment, determining the ability of the test compound to modulate the activity of FCTRX protein can be accomplished by determining the ability of the FCTRX protein further modulate an FCTRX target molecule. For example, the catalytic/enzymatic activity of the target molecule on an appropriate substrate can be determined as described, supra.

In yet another embodiment, the cell-free assay comprises contacting the FCTRX protein or biologically-active portion thereof with a known compound which binds FCTRX protein to form an assay mixture, contacting the assay mixture with a test compound, and determining the ability of the test compound to interact with an FCTRX protein, wherein determining the ability of the test compound to interact with an FCTRX protein comprises determining the ability of the FCTRX protein to preferentially bind to or modulate the activity of an FCTRX target molecule.

The cell-free assays of the invention are amenable to use of both the soluble form or the membrane-bound form of FCTRX protein. In the case of cell-free assays comprising the membrane-bound form of FCTRX protein, it may be desirable to utilize a solubilizing agent such that the membrane-bound form of FCTRX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl--N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either FCTRX protein or its target molecule to facilitate separation of complexed from uncomplexed forms of one or both of the proteins, as well as to accommodate automation of the assay. Binding of a test compound to FCTRX protein, or interaction of FCTRX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-FCTRX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or FCTRX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of FCTRX protein binding or activity determined using standard techniques.

Other techniques for immobilizing proteins on matrices can also be used in the screening assays of the invention. For example, either the FCTRX protein or its target molecule can be immobilized utilizing conjugation of biotin and streptavidin. Biotinylated FCTRX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with FCTRX protein or target molecules, but which do not interfere with binding of the FCTRX protein to its target molecule, can be derivatized to the wells of the plate, and unbound target or FCTRX protein trapped in the wells by antibody conjugation. Methods for detecting such complexes, in addition to those described above for the GST-immobilized complexes, include immunodetection of complexes using antibodies reactive with the FCTRX protein or target molecule, as well as enzyme-linked assays that rely on detecting an enzymatic activity associated with the FCTRX protein or target molecule.

In another embodiment, modulators of FCTRX protein expression are identified in a method wherein a cell is contacted with a candidate compound and the expression of FCTRX mRNA or protein in the cell is determined. The level of expression of FCTRX mRNA or protein in the presence of the candidate compound is compared to the level of expression of FCTRX mRNA or protein in the absence of the candidate compound. The candidate compound can then be identified as a modulator of FCTRX mRNA or protein expression based upon this comparison. For example, when expression of FCTRX mRNA or protein is greater (i.e., statistically significantly greater) in the presence of the candidate compound than in its absence, the candidate compound is identified as a stimulator of FCTRX mRNA or protein expression. Alternatively, when expression of FCTRX mRNA or protein is less (statistically significantly less) in the presence of the candidate compound than in its absence, the candidate compound is identified as an inhibitor of FCTRX mRNA or protein expression. The level of FCTRX mRNA or protein expression in the cells can be determined by methods described herein for detecting FCTRX mRNA or protein.

In yet another aspect of the invention, the FCTRX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with FCTRX (“FCTRX-binding proteins” or “FCTRX-bp”) and modulate FCTRX activity. Such FCTRX-binding proteins are also likely to be involved in the propagation of signals by the FCTRX proteins as, for example, upstream or downstream elements of the FCTRX pathway.

The two-hybrid system is based on the modular nature of most transcription factors, which consist of separable DNA-binding and activation domains. Briefly, the assay utilizes two different DNA constructs. In one construct, the gene that codes for FCTRX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an FCTRX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with FCTRX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of the FCTRX sequences, SEQ ID NOS: 1, 3, 5, 7, 9, 10, 11, 12, 14, 16, 18, 20, 22, and 24, or fragments or derivatives thereof, can be used to map the location of the FCTRX genes, respectively, on a chromosome. The mapping of the FCTRX sequences to chromosomes is an important first step in correlating these sequences with genes associated with disease.

Briefly, FCTRX genes can be mapped to chromosomes by preparing PCR primers (preferably 15-25 bp in length) from the FCTRX sequences. Computer analysis of the FCTRX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the FCTRX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the FCTRX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the FCTRX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The FCTRX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the FCTRX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The FCTRX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If predicted coding sequences, such as those in SEQ ID NOS:1, 3, 5, 7, 9, 10, 11, 12, 14, 16, 18, 20, 22, and 24, are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining FCTRX protein and/or nucleic acid expression as well as FCTRX activity, in the context of a biological sample (e.g., blood, serum, cells, tissue) to thereby determine whether an individual is .afflicted with a disease or disorder, or is at risk of developing a disorder, associated with aberrant FCTRX expression or activity. The disorders include Also within the scope of the invention is the use of a Therapeutic in the manufacture of a medicament for treating or preventing disorders or syndromes including, e.g., Colorectal cancer, adenomatous polyposis coli, myelogenous leukemia, congenital ceonatal alloimmune thrombocytopenia, multiple human solid malignancies, malignant ovarian tumours particularly at the interface between epithelia and stroma, malignant brain tumors, mammary tumors, human gliomas, astrocytomas, mixed glioma/astrocytomas, renal cells carcinoma, breast adenocarcinoma, ovarian cancer, melanomas, renal cell carcinoma, clear cell and granular cell carcinomas, autocrine/paracrine stimulation of tumor cell proliferation, autocrine/paracrine stimulation of tumor cell survival and tumor cell resistance to cytotoxic therapy, paranechmal and basement membrane invasion and motility of tumor cells thereby contributing to metastasis, tumor-mediated immunosuppression of T-cell mediated immune effector cells and pathways resulting in tumor escape from immune surveilance, neurological disorders, neurodegenerative disorders, nerve trauma, familial myelodysplastic syndrome, Charcot-Marie-Tooth neuropathy, demyelinating Gardner syndrome, familial myelodysplastic syndrome; mental health conditions, immunological disorders, allergy and infection, asthma, bronchial asthma, Avellino type eosinophilia, lung diseases, reproductive disorders, male infertility, female reproductive system disorders, male and female reproductive diseases, hemangioma, deafness, glycoprotein Ia deficiency, desmoid disease, turcot syndrome, liver cirrhosis, hepatitis C, gastric disorders, pancreatic diseases like diabetes, Schistosoma mansoni infection, Spinocerebellar ataxia, Plasmodium falciparum parasitemia, Corneal dystrophy—Groenouw type 1, Corneal dystrophy—lattice type I, and Reis-Bucklers corneal dystrophy. The invention also provides for prognostic (or predictive) assays for determining whether an individual is at risk of developing a disorder associated with FCTRX protein, nucleic acid expression or activity. For example, mutations in an FCTRX gene can be assayed in a biological sample. Such assays can be used for prognostic or predictive purpose to thereby prophylactically treat an individual prior to the onset of a disorder characterized by or associated with FCTRX protein, nucleic acid expression, or biological activity.

Another aspect of the invention provides methods for determining FCTRX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of FCTRX in clinical trials.

These and other agents are described in further detail in the following sections.

Diagnostic Assays

An exemplary method for detecting the presence or absence of FCTRX in a biological sample involves obtaining a biological sample from a test subject and contacting the biological sample with a compound or an agent capable of detecting FCTRX protein or nucleic acid (e.g., mRNA, genomic DNA) that encodes FCTRX protein such that the presence of FCTRX is detected in the biological sample. An agent for detecting FCTRX mRNA or genomic DNA is a labeled nucleic acid probe capable of hybridizing to FCTRX mRNA or genomic DNA. The nucleic acid probe can be, for example, a full-length FCTRX nucleic acid, such as the nucleic acid of SEQ ID NOS:1, 3, 5, 7, 9, 10, 11, 12, 14, 16, 18, 20, 22, and 24, or a portion thereof, such as an oligonucleotide of at least 15, 30, 50, 100, 250 or 500 nucleotides in length and sufficient to specifically hybridize under stringent conditions to FCTRX mRNA or genomic DNA. Other suitable probes for use in the diagnostic assays of the invention are described herein.

An agent for detecting FCTRX protein is an antibody capable of binding to FCTRX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect FCTRX mRNA, protein, or genomic DNA in a biological sample in vitro as well as in vivo. For example, in vitro techniques for detection of FCTRX mRNA include Northern hybridizations and in situ hybridizations. In vitro techniques for detection of FCTRX protein include enzyme linked immunosorbent assays (ELISAs), Western blots, immunoprecipitations, and immunofluorescence. In vitro techniques for detection of FCTRX genomic DNA include Southern hybridizations. Furthermore, in vivo techniques for detection of FCTRX protein include introducing into a subject a labeled anti-FCTRX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting FCTRX protein, mRNA, or genomic DNA, such that the presence of FCTRX protein, mRNA or genomic DNA is detected in the biological sample, and comparing the presence of FCTRX protein, mRNA or genomic DNA in the control sample with the presence of FCTRX protein, mRNA or genomic DNA in the test sample.

The invention also encompasses kits for detecting the presence of FCTRX in a biological sample. For example, the kit can comprise: a labeled compound or agent capable of detecting FCTRX protein or mRNA in a biological sample; means for determining the amount of FCTRX in the sample; and means for comparing the amount of FCTRX in the sample with a standard. The compound or agent can be packaged in a suitable container. The kit can further comprise instructions for using the kit to detect FCTRX protein or nucleic acid.

Prognostic Assays

The diagnostic methods described herein can furthermore be utilized to identify subjects having or at risk of developing a disease or disorder associated with aberrant FCTRX expression or activity. For example, the assays described herein, such as the preceding diagnostic assays or the following assays, can be utilized to identify a subject having or at risk of developing a disorder associated with FCTRX protein, nucleic acid expression or activity. Alternatively, the prognostic assays can be utilized to identify a subject having or at risk for developing a disease or disorder. Thus, the invention provides a method for identifying a disease or disorder associated with aberrant FCTRX expression or activity in which a test sample is obtained from a subject and FCTRX protein or nucleic acid (e.g., mRNA, genomic DNA) is detected, wherein the presence of FCTRX protein or nucleic acid is diagnostic for a subject having or at risk of developing a disease or disorder associated with aberrant FCTRX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant FCTRX expression or activity. For example, such methods can be used to determine whether a subject can be effectively treated with an agent for a disorder. Thus, the invention provides methods for determining whether a subject can be effectively treated with an agent for a disorder associated with aberrant FCTRX expression or activity in which a test sample is obtained and FCTRX protein or nucleic acid is detected (e.g., wherein the presence of FCTRX protein or nucleic acid is diagnostic for a subject that can be administered the agent to treat a disorder associated with aberrant FCTRX expression or activity).

The methods of the invention can also be used to detect genetic lesions in an FCTRX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding an FCTRX-protein, or the misexpression of the FCTRX gene. For example, such genetic lesions can be detected by ascertaining the existence of at least one of: (i) a deletion of one or more nucleotides from an FCTRX gene; (ii) an addition of one or more nucleotides to an FCTRX gene; (iii) a substitution of one or more nucleotides of an FCTRX gene, (iv) a chromosomal rearrangement of an FCTRX gene; (v) an alteration in the level of a messenger RNA transcript of an FCTRX gene, (vi) aberrant modification of an FCTRX gene, such as of the methylation pattern of the genomic DNA, (vii) the presence of a non-wild-type splicing pattern of a messenger RNA transcript of an FCTRX gene, (viii) a non-wild-type level of an FCTRX protein, (ix) allelic loss of an FCTRX gene, and (x) inappropriate post-translational modification of an FCTRX protein. As described herein, there are a large number of assay techniques known in the art which can be used for detecting lesions in an FCTRX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the FCTRX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to an FCTRX gene under conditions such that hybridization and amplification of the FCTRX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in an FCTRX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in FCTRX can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in FCTRX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the FCTRX gene and detect mutations by comparing the sequence of the sample FCTRX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the FCTRX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type FCTRX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S₁ nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in FCTRX cDNAs obtained from samples of cells. For example, the mutY enzyme of E. coli cleaves A at GIA mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on an FCTRX sequence, e.g., a wild-type FCTRX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in FCTRX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control FCTRX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving an FCTRX gene.

Furthermore, any cell type or tissue, preferably peripheral blood leukocytes, in which FCTRX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on FCTRX activity (e.g., FCTRX gene expression), as identified by a screening assay described herein can be administered to individuals to treat (prophylactically or therapeutically) disorders (The disorders include metabolic disorders, Also within the scope of the invention is the use of a Therapeutic in the manufacture of a medicament for treating or preventing disorders or syndromes including, e.g., Colorectal cancer, adenomatous polyposis coli, myelogenous leukemia, congenital ceonatal alloimmune thrombocytopenia, multiple human solid malignancies, malignant ovarian tumours particularly at the interface between epithelia and stroma, malignant brain tumors, mammary tumors, human gliomas, astrocytomas, mixed glioma/astrocytomas, renal cells carcinoma, breast adenocarcinoma, ovarian cancer, melanomas, renal cell carcinoma, clear cell and granular cell carcinomas, autocrine/paracrine stimulation of tumor cell proliferation, autocrine/paracrine stimulation of tumor cell survival and tumor cell resistance to cytotoxic therapy, paranechmal and basement membrane invasion and motility of tumor cells thereby contributing to metastasis, tumor-mediated immunosuppression of T-cell mediated immune effector cells and pathways resulting in tumor escape from immune surveilance, neurological disorders, neurodegenerative disorders, nerve trauma, familial myelodysplastic syndrome, Charcot-Marie-Tooth neuropathy, demyelinating Gardner syndrome, familial myelodysplastic syndrome; mental health conditions, immunological disorders, allergy and infection, asthma, bronchial asthma, Avellino type eosinophilia, lung diseases, reproductive disorders, male infertility, female reproductive system disorders, male and female reproductive diseases, hemangioma, deafness, glycoprotein Ia deficiency, desmoid disease, turcot syndrome, liver cirrhosis, hepatitis C, gastric disorders, pancreatic diseases like diabetes, Schistosoma mansoni infection, Spinocerebellar ataxia, Plasmodium falciparum parasitemia, Corneal dystrophy—Groenouw type I, Corneal dystrophy—lattice type I, and Reis-Bucklers corneal dystrophy) In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of FCTRX protein, expression of FCTRX nucleic acid, or mutation content of FCTRX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of FCTRX protein, expression of FCTRX nucleic acid, or mutation content of FCTRX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with an FCTRX modulator, such as a modulator identified by one of the exemplary screening assays described herein.

Monitoring of Effects During Clinical Trials

Monitoring the influence of agents (e.g., drugs, compounds) on the expression or activity of FCTRX (e.g., the ability to modulate aberrant cell proliferation and/or differentiation) can be applied not only in basic drug screening, but also in clinical trials. For example, the effectiveness of an agent determined by a screening assay as described herein to increase FCTRX gene expression, protein levels, or upregulate FCTRX activity, can be monitored in clinical trails of subjects exhibiting decreased FCTRX gene expression, protein levels, or downregulated FCTRX activity. Alternatively, the effectiveness of an agent determined by a screening assay to decrease FCTRX gene expression, protein levels, or downregulate FCTRX activity, can be monitored in clinical trails of subjects exhibiting increased FCTRX gene expression, protein levels, or upregulated FCTRX activity. In such clinical trials, the expression or activity of FCTRX and, preferably, other genes that have been implicated in, for example, a cellular proliferation or immune disorder can be used as a “read out” or markers of the immune responsiveness of a particular cell.

By way of example, and not of limnitation, genes, including FCTRX, that are modulated in cells by treatment with an agent (e.g., compound, drug or small molecule) that modulates FCTRX activity (e.g., identified in a screening assay as described herein) can be identified. Thus, to study the effect of agents on cellular proliferation disorders, for example, in a clinical trial, cells can be isolated and RNA prepared and analyzed for the levels of expression of FCTRX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of FCTRX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of an FCTRX protein, mRNA, or genomic DNA in the preadministration sample; (iii) obtaining one or more post-administration samples from the subject; (iv) detecting the level of expression or activity of the FCTRX protein, mRNA, or genomic DNA in the post-administration samples; (v) comparing the level of expression or activity of the FCTRX protein, mRNA, or genomic DNA in the pre-administration sample with the FCTRX protein, mRNA, or genomic DNA in the post administration sample or samples; and (vi) altering the administration of the agent to the subject accordingly. For example, increased administration of the agent may be desirable to increase the expression or activity of FCTRX to higher levels than detected, i.e., to increase the effectiveness of the agent. Alternatively, decreased administration of the agent may be desirable to decrease expression or activity of FCTRX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant FCTRX expression or activity. The disorders include cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, prostate cancer, neoplasm; adenocarcinoma, lymphoma, uterus cancer, fertility, hemophilia, hypercoagulation, idiopathic thrombocytopenic purpura, immunodeficiencies, graft versus host disease, AIDS, bronchial asthma, Crohn's disease; multiple sclerosis, treatment of Albright Hereditary Ostoeodystrophy, and other diseases, disorders and conditions of the like.

These methods of treatment will be discussed more fully, below.

Disease and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endoggenous function of an aforementioned peptide by homologous recombination (see, e.g., Capecchi, 1989. Science 244: 1288-1292); or (v) modulators (i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof, or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant FCTRX expression or activity, by administering to the subject an agent that modulates FCTRX expression or at least one FCTRX activity. Subjects at risk for a disease that is caused or contributed to by aberrant FCTRX expression or activity can be identified by, for example, any or a combination of diagnostic or prognostic assays as described herein. Administration of a prophylactic agent can occur prior to the manifestation of symptoms characteristic of the FCTRX aberrancy, such that a disease or disorder is prevented or, alternatively, delayed in its progression. Depending upon the type of FCTRX aberrancy, for example, an FCTRX agonist or FCTRX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating FCTRX expression or activity for therapeutic purposes. The modulatory method of the invention involves contacting a cell with an agent that modulates one or more of the activities of FCTRX protein activity associated with the cell. An agent that modulates FCTRX protein activity can be an agent as described herein, such as a nucleic acid or a protein, a naturally-occurring cognate ligand of an FCTRX protein, a peptide, an FCTRX peptidomimetic, or other small molecule. In one embodiment, the agent stimulates one or more FCTRX protein activity. Examples of such stimulatory agents include active FCTRX protein and a nucleic acid molecule encoding FCTRX that has been introduced into the cell. In another embodiment, the agent inhibits one or more FCTRX protein activity. Examples of such inhibitory agents include antisense FCTRX nucleic acid molecules and anti-FCTRX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of an FCTRX protein or nucleic acid molecule. In one embodiment, the method involves administering an agent (e.g., an agent identified by a screening assay described herein), or combination of agents that modulates (e.g., up-regulates or down-regulates) FCTRX expression or activity. In another embodiment, the method involves administering an FCTRX protein or nucleic acid molecule as therapy to compensate for reduced or aberrant FCTRX expression or activity.

Stimulation of FCTRX activity is desirable in situations in which FCTRX is abnormally downregulated and/or in which increased FCTRX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The FCTRX nucleic acids and proteins of the invention are useful in potential prophylactic and therapeutic applications implicated in a variety of disorders including, but not limited to: Also within the scope of the invention is the use of a Therapeutic in the manufacture of a medicament for treating or preventing disorders or syndromes including, e.g., Colorectal cancer, adenomatous polyposis coli, myelogenous leukemia, congenital ceonatal alloimmune thrombocytopenia, multiple human solid malignancies, malignant ovarian tumours particularly at the interface between epithelia and stroma, malignant brain tumors, mammary tumors, human gliomas, astrocytomas, mixed glioma/astrocytomas, renal cells carcinoma, breast adenocarcinoma, ovarian cancer, melanomas, renal cell carcinoma, clear cell and granular cell carcinomas, autocrine/paracrine stimulation of tumor cell proliferation, autocrine/paracrine stimulation of tumor cell survival and tumor cell resistance to cytotoxic therapy, paranechmal and basement membrane invasion and motility of tumor cells thereby contributing to metastasis, tumor-mediated immunosuppression of T-cell mediated immune effector cells and pathways resulting in tumor escape from immune surveilance, neurological disorders, neurodegenerative disorders, nerve trauma, familial myelodysplastic syndrome, Charcot-Marie-Tooth neuropathy, demyelinating Gardner syndrome, familial myelodysplastic syndrome; mental health conditions, immunological disorders, allergy and infection, asthma, bronchial asthma, Avellino type eosinophilia, lung diseases, reproductive disorders, male infertility, female reproductive system disorders, male and female reproductive diseases, hemangioma, deafness, glycoprotein Ia deficiency, desmoid disease, turcot syndrome, liver cirrhosis, hepatitis C, gastric disorders, pancreatic diseases like diabetes, Schistosoma mansoni infection, Spinocerebellar ataxia, Plasmodium falciparum parasitemia, Corneal dystrophy—Groenouw type I, Corneal dystrophy—lattice type I, and Reis-Bucklers corneal dystrophy.

As an example, a cDNA encoding the FCTRX protein of the invention may be useful in gene therapy, and the protein may be useful when administered to a subject in need thereof. By way of non-limiting example, the compositions of the invention will have efficacy for treatment of patients suffering from: Also within the scope of the invention is the use of a Therapeutic in the manufacture of a medicament for treating or preventing disorders or syndromes including, e.g., Colorectal cancer, adenomatous polyposis coli, myelogenous leukemia, congenital ceonatal alloimmune thrombocytopenia, multiple human solid malignancies, malignant ovarian tumours particularly at the interface between epithelia and stroma, malignant brain tumors, mammary tumors, human gliomas, astrocytomas, mixed glioma/astrocytomas, renal cells carcinoma, breast adenocarcinoma, ovarian cancer, melanomas, renal cell carcinoma, clear cell and granular cell carcinomas, autocrine/paracrine stimulation of tumor cell proliferation, autocrine/paracrine stimulation of tumor cell survival and tumor cell resistance to cytotoxic therapy, paranechmal and basement membrane invasion and motility of tumor cells thereby contributing to metastasis, tumor-mediated immunosuppression of T-cell mediated immune effector cells and pathways resulting in tumor escape from immune surveilance, neurological disorders, neurodegenerative disorders, nerve trauma, familial myelodysplastic syndrome, Charcot-Marie-Tooth neuropathy, demyelinating Gardner syndrome, familial myelodysplastic syndrome; mental health conditions, immunological disorders, allergy and infection, asthma, bronchial asthma, Avellino type eosinophilia, lung diseases, reproductive disorders, male infertility, female reproductive system disorders, male and female reproductive diseases, hemangioma, deafness, glycoprotein Ia deficiency, desmoid disease, turcot syndrome, liver cirrhosis, hepatitis C, gastric disorders, pancreatic diseases like diabetes, Schistosoma mansoni infection, Spinocerebellar ataxia, Plasmodium falciparum parasitemia, Corneal dystrophy—Groenouw type I, Corneal dystrophy—lattice type I, and Reis-Bucklers corneal dystrophy.

Both the novel nucleic acid encoding the FCTRX protein, and the FCTRX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies which immunospecifically-bind to the novel substances of the invention for use in therapeutic or diagnostic methods.

### EXAMPLES

The following examples illustrate by way of non-limiting example various aspects of the invention.

The following examples illustrate by way of non-limiting example various aspects of the invention.

### Example 1

### Method of Identifying the Nucleic Acids

The novel nucleic acids of the invention were identified by TblastN using a proprietary sequence file, run against the Genomic Daily Files made available by GenBank. The nucleic acids were further predicted by the proprietary software program GenScan™, including selection of exons. These were further modified by means of similarities using BLAST searches. The sequences were then manually corrected for apparent inconsistencies, thereby obtaining the sequences encoding the full-length proteins.

### Example 2

### Quantitative Expression Analysis of FCTR2 in Various Cells and Tissues

The quantitative expression of various clones was assessed using microtiter plates containing RNA samples from a variety of normal and pathology-derived cells, cell lines and tissues using real time quantitative PCR (RTQ PCR; TAQMAN®). RTQ PCR was performed on a Perkin-Elmer Biosystems ABI PRISM® 7700 Sequence Detection System. Various collections of samples are assembled on the plates, and referred to as Panel 1 (containing cells and cell lines from normal and cancer sources), Panel 2 (containing samples derived from tissues, in particular from surgical samples, from normal and cancer sources), Panel 3 (containing samples derived from a wide variety of cancer sources) and Panel 4 (containing cells and cell lines from normal cells and cells related to inflammatory conditions).

First, the RNA samples were normalized to constitutively expressed genes such as β-actin and GAPDH. RNA (˜50 ng total or ˜1 ng polyA+) was converted to cDNA using the TAQMAN® Reverse Transcription Reagents Kit (PE Biosystems, Foster City, Calif.; Catalog No. N808-0234) and random hexamers according to the manufacturer's protocol. Reactions were performed in 20 ul and incubated for 30 min. at 48° C. cDNA (5 ul) was then transferred to a separate plate for the TAQMAN® reaction using β-actin and GAPDH TAQMAN® Assay Reagents (PE Biosystems; Catalog Nos. 4310881E and 4310884E, respectively) and TAQMAN® universal PCR Master Mix (PE Biosystems; Catalog No. 4304447) according to the manufacturer's protocol. Reactions were performed in 25 ul using the following parameters: 2 min. at 50° C.; 10 min. at 95° C.; 15 sec. at 95° C./1 min. at 60° C. (40 cycles). Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100. The average CT values obtained for β-actin and GAPDH were used to normalize RNA samples. The RNA sample generating the highest CT value required no further diluting, while all other samples were diluted relative to this sample according to their β-actin /GAPDH average CT values.

Normalized RNA (5 ul) was converted to cDNA and analyzed via TAQMAN® using One Step RT-PCR Master Mix Reagents (PE Biosystems; Catalog No. 4309169) and gene-specific primers according to the manufacturer's instructions. Probes and primers were designed for each assay according to Perkin Elmer Biosystem's Primer Express Software package (version I for Apple Computer's Macintosh Power PC) or a similar algorithm using the target sequence as input. Default settings were used for reaction conditions and the following parameters were set before selecting primers: primer concentration=250 nM, primer melting temperature (Tₘ) range=58°-60° C., primer optimal Tm=59° C., maximum primer difference=2° C., probe does not have 5′ G, probe Tₘ must be 10° C. greater than primer Tₘ, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200nM.

PCR conditions: Normalized RNA from each tissue and each cell line was spotted in each well of a 96 well PCR plate (Perkin Elmer Biosystems). PCR cocktails including two probes (a probe specific for the target clone and another gene-specific probe multiplexed with the target probe) were set up using 1× TaqMan™ PCR Master Mix for the PE Biosystems 7700, with 5 mM MgCl2, dNTPs (dA, G, C, U at 1:1:1:2 ratios), 0.25 U/ml AmpliTaq Gold™ (PE Biosystems), and 0.4 U/μl RNase inhibitor, and 0.25 U/el reverse transcriptase. Reverse transcription was performed at 48° C. for 30 minutes followed by amplification/PCR cycles as follows: 95° C. 10 min, then 40 cycles of 95° C. for 15 seconds, 60° C. for 1 minute.

In the results for Panel 1, the following abbreviations are used:

ca.=carcinoma,

*=established from metastasis,

met=metastasis,

s cell var=small cell variant,

non-s=non-sm=non-small,

squam=squamous,

pl. eff=pl effusion=pleural effusion,

glio=glioma,

astro=astrocytoma, and

neuro=neuroblastoma.

Panel 2

The plates for Panel 2 generally include 2 control wells and 94 test samples composed of RNA or cDNA isolated from human tissue procured by surgeons working in close cooperation with the National Cancer Institute's Cooperative Human Tissue Network (CHTN) or the National Disease Research Initiative (NDRI). The tissues are derived from human malignancies and in cases where indicated many malignant tissues have “matched margins” obtained from noncancerous tissue just adjacent to the tumor. These are termed normal adjacent tissues and are denoted “NAT” in the results below. The tumor tissue and the “matched margins” are evaluated by two independent pathologists (the surgical pathologists and again by a pathologists at NDRI or CHTN). This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue, in Table RR). In addition, RNA and cDNA samples were obtained from various human tissues derived from autopsies performed on elderly people or sudden death victims (accidents, etc.). These tissue were ascertained to be free of disease and were purchased from various commercial sources such as Clontech (Palo Alto, Calif.), Research Genetics, and Invitrogen.

RNA integrity from all samples is controlled for quality by visual assessment of agarose gel electropherograms using 28S and 18S ribosomal RNA staining intensity ratio as a guide (2:1 to 2.5:1 28s: 18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

Panel 4

Panel 4 includes samples on a 96 well plate (2 control wells, 94 test samples) composed of RNA (Panel 4r) or cDNA (Panel 4d) isolated from various human cell lines or tissues related to inflammatory conditions. Total RNA from control normal tissues such as colon and lung (Stratagene, La Jolla, Calif.) and thymus and kidney (Clontech) were employed. Total RNA from liver tissue from cirrhosis patients and kidney from lupus patients was obtained from BioChain (Biochain Institute, Inc., Hayward, Calif.). Intestinal tissue for RNA preparation from patients diagnosed as having Crohn's disease and ulcerative colitis was obtained from the National Disease Research Interchange (NDRI) (Philadelphia, Pa.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics (Walkersville, Md.) and grown in the media supplied for these cell types by Clonetics. These primary cell types were activated with various cytokines or combinations of cytokines for 6 and/or 12-14 hours, as indicated. The following cytokines were used; IL-1 beta at approximately 1-5 ng/ml, TNF alpha at approximately 5-10 ng/ml, IFN gamma at approximately 20-50 ng/ml, IL-4 at approximately 5-10 ng/ml, IL-9 at approximately 5-10 ng/ml, IL-13 at approximately 5-10 ng/ml. Endothelial cells were sometimes starved for various times by culture in the basal media from Clonetics with 0.1% serum.

Mononuclear cells were prepared from blood of employees at CuraGen Corporation, using Ficoll. LAK cells were prepared from these cells by culture in DMEM 5% FCS (Hyclone), 100 μM non essential amino acids (Gibco/Life Technologies, Rockville, Md.), 1 mM sodium pyruvate (Gibco), mercaptoethanol 5.5×10⁻⁵ M (Gibco), and 10 mM Hepes (Gibco) and Interleukin 2 for 4-6 days. Cells were then either activated with 10-20 ng/ml PMA and 1-2 μg/ml ionomycin, IL-12 at 5-10 ng/ml, IFN gamma at 20-50 ng/ml and IL-18 at 5-10 ng/ml for 6 hours. In some cases, mononuclear cells were cultured for 4-5 days in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) with PHA (phytohemagglutinin) or PWM (pokeweed mitogen) at approximately 5 μg/ml. Samples were taken at 24, 48 and 72 hours for RNA preparation. MLR (mixed lymphocyte reaction) samples were obtained by taking blood from two donors, isolating the mononuclear cells using Ficoll and mixing the isolated mononuclear cells 1:1 at a final concentration of approximately 2×10⁶ cells/ml in DMEM 5% FCS (Hyclone), 100 μM non essential amino acids (Gibco), 1 mM sodium pyruvate (Gibco), mercaptoethanol (5.5×10⁻⁵ M) (Gibco), and 10 mM Hepes (Gibco). The MLR was cultured and samples taken at various time points ranging from 1-7 days for RNA preparation.

Monocytes were isolated from mononuclear cells using CD14 Miltenyi Beads, +ve VS selection columns and a Vario Magnet according to the manufacturer's instructions. Monocytes were differentiated into dendritic cells by culture in DMEM 5% fetal calf serum (FCS) (Hyclone, Logan, Utah), 100 μM non essential amino acids (Gibco), 1 mM sodium pyruvate (Gibco), mercaptoethanol 5.5×10⁻⁵ M (Gibco), and 10 mM Hepes (Gibco), 50 ng/ml GMCSF and 5 ng/ml IL-4 for 5-7 days. Macrophages were prepared by culture of monocytes for 5-7 days in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and 10% AB Human Serum or MCSF at approximately 50 ng/ml. Monocytes, macrophages and dendritic cells were stimulated for 6 and 12-14 hours with lipopolysaccharide (LPS) at 100 ng/ml. Dendritic cells were also stimulated with anti-CD40 monoclonal antibody (Pharmingen) at 10 μg/ml for 6 and 12-14 hours.

CD4 lymphocytes, CD8 lymphocytes and NK cells were also isolated from mononuclear cells using CD4, CD8 and CD56 Miltenyi beads, positive VS selection columns and a Vario Magnet according to the manufacturer's instructions. CD45RA and CD45RO CD4 lymphocytes were isolated by depleting mononuclear cells of CD8, CD56, CD14 and CD19 cells using CD8, CD56, CD14 and CD19 Miltenyi beads and +ve selection. Then CD45RO beads were used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and plated at 10⁶ cells/ml onto Falcon 6 well tissue culture plates that had been coated overnight with 0.5 μg/ml anti-CD28 (Pharmingen) and 3 ug/ml anti-CD3 (OKT3, ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and IL-2. The expanded CD8 cells were then activated again with plate bound anti-CD3 and anti-CD28 for 4 days and expanded as before. RNA was isolated 6 and 24 hours after the second activation and after 4 days of the second expansion culture. The isolated NK cells were cultured in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and IL-2 for 4-6 days before RNA was prepared.

To obtain B cells, tonsils were procured from NDRI. The tonsil was cut up with sterile dissecting scissors and then passed through a sieve. Tonsil cells were then spun down and resupended at 10⁶ cells/ml in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco). To activate the cells, we used PWM at 5 μg/ml or anti-CD40 (Pharmingen) at approximately 10 μg/ml and IL-4 at 5-10 ng/ml. Cells were harvested for RNA preparation at 24, 48 and 72 hours.

To prepare the primary and secondary Th1/Th2 and Tr1 cells, six-well Falcon plates were coated overnight with 10 μg/ml anti-CD28 (Pharmingen) and 2 μg/ml OKT3 (ATCC), and then washed twice with PBS. Umbilical cord blood CD4 lymphocytes (Poietic Systems, German Town, Md.) were cultured at 10⁵-10⁶ cells/ml in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and IL-2 (4 ng/ml). IL-12 (5 ng/ml) and anti-IL4 (1 μg/ml) were used to direct to Th1, while IL-4 (5 ng/ml) and anti-IFN gamma (1 μg/ml) were used to direct to Th2 and IL-10 at 5 ng/ml was used to direct to Tr1. After 4-5 days, the activated Th1, Th2 and Tr1 lymphocytes were washed once in DMEM and expanded for 4-7 days in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and IL-2 (1 ng/ml). Following this, the activated Th1, Th2 and Tr1 lymphocytes were restimulated for 5 days with anti-CD28/OKT3 and cytokines as described above, but with the addition of anti-CD95L (1 ,ug/ml) to prevent apoptosis. After 4-5 days, the Th1, Th2 and Tr1 lymphocytes were washed and then expanded again with IL-2 for 4-7 days. Activated Th1 and Th2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture in 0.1 mM dbcAMP at 5×10⁵ cells/ml for 8 days, changing the media every 3 days and adjusting the cell concentration to 5×10⁵ cells/ml. For the culture of these cells, we used DMEM or RPMI (as recommended by the ATCC), with the addition of 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco). RNA was either prepared from resting cells or cells activated with PMA at 10 ng/ml and ionomycin at 1 μg/ml for 6 and 14 hours. Keratinocyte line CCD106 and an airway epithelial tumor line NCI-H292 were also obtained from the ATCC. Both were cultured in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco). CCD1106 cells were activated for 6 and 14 hours with approximately 5 ng/ml TNF alpha and 1 ng/ml IL-1 beta, while NCI-H292 cells were activated for 6 and 14 hours with the following cytokines: 5 ng/ml IL-4, 5 ng/ml IL-9, 5 ng/ml IL-13 and 25 ng/ml IFN gamma.

For these cell lines and blood cells, RNA was prepared by lysing approximately 10⁷ cells/ml using Trizol (Gibco BRL). Briefly, 1/10 volume of bromochloropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at −20 degrees C. overnight. The precipitated RNA was spun down at 9,000 rpm for 15 min in a Sorvall SS34 rotor and washed in 70% ethanol. The pellet was redissolved in 300 μl of RNAse-free water and 35 μl buffer (Promega) 5 μl DTT, 7 μl RNAsin and 8 μl DNAse were added. The tube was incubated at 37 degrees C. for 30 minutes to remove contaminating genomic DNA, extracted once with phenol chloroform and re-precipitated with 1/10 volume of 3 M sodium acetate and 2 volumes of 100% ethanol. The RNA was spun down and placed in RNAse free water. RNA was stored at −80 degrees C.

The above detailed procedures were carried out to obtain the taqman profiles of the clones in question.

Given below are the Primers and the Taqman results for the following clones:

58092213.0.36—Probe Name: Ag809 (Table 9 and Table 10)

29692275.0.1—Probe Name: Ag2773 (Table 11 and Table 12)

32125243.0.21—Probe Name: Ag427 (Table 13 and Table 14)

27455183.0.19—Probe Name: Ag1541 (Table 15 and Table 16, 17, 18)

TABLE 8
Primer Design for Probe Ag809 (FCTR1)

| | | | | Start | SEQID |
|---|---|---|---|---|---|
| Primer | Sequences | TM | Length | Pos | NO |
| Forward | 5′-ATGTGATCTTTGGCTGTGAAGT-3′ | 58.7 | 22 | 337 | 24 |
| Probe | FAM-5′-CTACCCCATGGCCTCCATCGAGT-3′-TAMRA | 69.4 | 23 | 365 | 25 |
| Reverse | 5′-GGATGTCCAAGCCATCCTT-3′ | 59.9 | 19 | 393 | 26 |

TABLE 9
TAQMAN RESULTS FOR FCTR1

| | Panel | | Panel | | Panel |
|---|---|---|---|---|---|
| Tissue_Name | 1 | Tissue_Name | 2D | Tissue_Name | 4D |
| Liver | 79.6 | Normal Colon | 6.8 | 93768_Secondary Th1_anti- | 2.0 |
| adenocarcinoma | | GENPAK | | CD28/anti-CD3 | |
| | | 061003 | | | |
| Heart (fetal) | 43.8 | 83219 CC Well | 6.1 | 93769_Secondary Th2_anti- | 1.5 |
| | | to Mod Diff | | CD28/anti-CD3 | |
| | | (ODO3866) | | | |
| Pancreas | 2.1 | 83220 CC NAT | 2.5 | 93770_Secondary Th1_anti- | 2.5 |
| | | (ODO3866) | | CD28/anti-CD3 | |
| Pancreatic ca. | 4.7 | 83221 CC Gr.2 | 0.9 | 93573_Secondary Th1_resting | 1.0 |
| CAPAN 2 | | rectosigmoid | | day 4-6 in IL-2 | |
| | | (ODO3868) | | | |
| Adrenal gland | 2.3 | 83222 CC NAT | 1.2 | 93572_Secondary Th2_resting | 3.0 |
| | | (ODO3868) | | day 4-6 in IL-2 | |
| Thyroid | 6.5 | 83235 CC Mod | 3.8 | 93571_Secondary Tr1_resting | 1.7 |
| | | Diff (ODO3920) | | day 4-6 in IL-2 | |
| Salivary gland | 12.3 | 83236 CC NAT | 1.3 | 93568_primary Th1_anti- | 0.4 |
| | | (ODO3920) | | CD28/anti-CD3 | |
| Pituitary gland | 8.7 | 83237 CC Gr.2 | 6.9 | 93569_primary Th2_anti- | 1.5 |
| | | ascend colon | | CD28/anti-CD3 | |
| | | (ODO3921) | | | |
| Brain (fetal) | 0.0 | 83238 CC NAT | 4.0 | 93570_primary Tr1_anti- | 2.0 |
| | | (ODO3921) | | CD28/anti-CD3 | |
| Brain (whole) | 3.0 | 83241 CC from | 1.2 | 93565_primary Th1_resting dy 4- | 5.4 |
| | | Partial | | 6 in IL-2 | |
| | | Hepatectomy | | | |
| | | (ODO4309) | | | |
| Brain (amygdala) | 2.4 | 83242 Liver NAT | 0.6 | 93566_primary Th2_resting dy 4- | 3.1 |
| | | (ODO4309) | | 6 in IL-2 | |
| Brain | 0.0 | 87472 Colon | 4.4 | 93567_primary Tr1_resting dy 4-6 | 0.0 |
| (cerebellum) | | mets to lung | | in IL-2 | |
| | | (OD04451-01) | | | |
| Brain | 13.0 | 87473 Lung NAT | 1.2 | 93351_CD45RA CD4 | 11.2 |
| (hippocampus) | | (OD04451-02) | | lymphocyte_anti-CD28/anti-CD3 | |
| Brain (thalamus) | 3.0 | Normal Prostate | 10.2 | 93352_CD45RO CD4 | 1.2 |
| | | Clontech A+ | | lymphocyte_anti-CD28/anti-CD3 | |
| | | 6546-1 | | | |
| Cerebral Cortex | 2.3 | 84140 Prostate | 41.8 | 93251_CD8 Lymphocytes_anti- | 0.9 |
| | | Cancer | | CD28/anti-CD3 | |
| | | (OD04410) | | | |
| Spinal cord | 2.6 | 84141 Prostate | 25.7 | 93353_chronic CD8 Lymphocytes | 0.0 |
| | | NAT (OD04410) | | 2ry_resting dy 4-6 in IL-2 | |
| CNS ca. | 12.1 | 87073 Prostate | 11.0 | 93574_chronic CD8 Lymphocytes | 0.6 |
| (glio/astro) U87- | | Cancer | | 2ry_activated CD3/CD28 | |
| MG | | (OD04720-01) | | | |
| CNS ca. | 100.0 | 87074 Prostate | 10.0 | 93354_CD4_none | 1.1 |
| (glio/astro) U- | | NAT (OD04720- | | | |
| 118-MG | | 02) | | | |
| CNS ca. (astro) | 6.5 | Normal Lung | 7.9 | 93252_Secondary | 0.0 |
| SW1783 | | GENPAK | | Th1/Th2/Tr1_anti-CD95 CH11 | |
| | | 061010 | | | |
| CNS ca.* (neuro; | 52.1 | 83239 Lung Met | 6.5 | 93103_LAK cells_resting | 0.5 |
| met) SK-N-AS | | to Muscle | | | |
| | | (ODO4286) | | | |
| CNS ca. (astro) | 12.6 | 83240 Muscle | 2.6 | 93788_LAK cells_IL-2 | 0.0 |
| SF-539 | | NAT (ODO4286) | | | |
| CNS ca. (astro) | 11.9 | 84136 Lung | 14.8 | 93787_LAK cells_IL-2 + IL-12 | 0.7 |
| SNB-75 | | Malignant | | | |
| | | Cancer | | | |
| | | (OD03126) | | | |
| CNS ca. | 0.0 | 84137 Lung NAT | 3.2 | 93789_LAK cells_IL-2 + IFN | 1.1 |
| (glio)SNB-19 | | (OD03126) | | gamma | |
| CNS ca. | 0.9 | 84871 Lung | 2.1 | 93790_LAK cells_IL-2 + IL-18 | 0.3 |
| (glio)U251 | | Cancer | | | |
| | | (OD04404) | | | |
| CNS ca. (glio) | 12.6 | 84872 Lung NAT | 1.9 | 93104_LAK cells_PMA/ionomycin | 0.0 |
| SF-295 | | (OD04404) | | and IL-18 | |
| Heart | 13.9 | 84875 Lung | 0.3 | 93578_NK Cells IL-2_resting | 1.3 |
| | | Cancer | | | |
| | | (OD04565) | | | |
| Skeletal muscle | 3.2 | 85950 Lung | 1.3 | 93109_Mixed Lymphocyte | 0.5 |
| | | Cancer | | Reaction_Two Way MLR | |
| | | (OD04237-01) | | | |
| Bone marrow | 3.6 | 85970 Lung NAT | 2.6 | 93110_Mixed Lymphocyte | 0.5 |
| | | (OD04237-02) | | Reaction_Two Way MLR | |
| Thymus | 4.2 | 83255 Ocular | 0.1 | 93111_Mixed Lymphocyte | 2.7 |
| | | Mel Met to Liver | | Reaction_Two Way MLR | |
| | | (ODO4310) | | | |
| Spleen | 61.6 | 83256 Liver NAT | 0.6 | 93112_Mononuclear Cells | 0.0 |
| | | (ODO4310) | | (PBMCs)_resting | |
| Lymph node | 3.3 | 84139 | 2.5 | 93113_Mononuclear Cells | 1.3 |
| | | Melanoma Mets | | (PBMCs)_PWM | |
| | | to Lung | | | |
| | | (OD04321) | | | |
| Colorectal | 11.9 | 84138 Lung | 2.6 | 93114_Mononuclear Cells | 1.0 |
| | | NAT (OD04321) | | (PBMCs)_PHA-L | |
| Stomach | 28.3 | Normal Kidney | 5.6 | 93249_Ramos (B cell)_none | 1.2 |
| | | GENPAK | | | |
| | | 061008 | | | |
| Small intestine | 4.5 | 83786 Kidney | 0.6 | 93250_Ramos (B cell)_ionomycin | 2.3 |
| | | Ca, Nuclear | | | |
| | | grade 2 | | | |
| | | (OD04338) | | | |
| Colon ca. SW480 | 46.7 | 83787 Kidney | 3.7 | 93349_B lymphocytes_PWM | 4.3 |
| | | NAT (OD04338) | | | |
| Colon ca.* | 19.0 | 83788 Kidney Ca | 0.8 | 93350_B lymphoytes_CD40L and | 1.4 |
| (SW480 | | Nuclear grade | | IL-4 | |
| met)SW620 | | 1/2 (OD04339) | | | |
| Colon ca. HT29 | 5.3 | 83789 Kidney | 3.1 | 92665_EOL-1 | 7.2 |
| | | NAT (OD04339) | | (Eosinophil)_dbcAMP | |
| | | | | differentiated | |
| Colon ca. HCT- | 5.0 | 83790 Kidney | 1.5 | 93248_EOL-1 | 3.0 |
| 116 | | Ca, Clear cell | | (Eosinophil)_dbcAMP/PMAiono- | |
| | | type (OD04340) | | mycin | |
| Colon ca. CaCo-2 | 49.3 | 83791 Kidney | 5.1 | 93356_Dendritic Cells_none | 1.5 |
| | | NAT (OD04340) | | | |
| 83219 CC Well to | 3.0 | 83792 Kidney | 14.5 | 93355_Dendritic Cells_LPS 100 | 0.7 |
| Mod Diff | | Ca, Nuclear | | ng/ml | |
| (ODO3866) | | grade 3 | | | |
| | | (OD04348) | | | |
| Colon ca. HCC- | 27.7 | 83793 Kidney | 2.5 | 93775_Dendritic Cells_anti-CD40 | 0.5 |
| 2998 | | NAT OD04348) | | | |
| Gastric ca.* (liver | 10.5 | 87474 Kidney | 1.7 | 93774_Monocytes_resting | 0.5 |
| met) NCI-N87 | | Cancer | | | |
| | | (OD04622-01) | | | |
| Bladder | 3.7 | 87475 Kidney | 2.0 | 93776_Monocytes_LPS 50 ng/ml | 0.0 |
| | | NAT (OD04622- | | | |
| | | 03) | | | |
| Trachea | 23.5 | 85973 Kidney | 0.3 | 93581_Macrophages_resting | 1.3 |
| | | Cancer | | | |
| | | (OD04450-01) | | | |
| Kidney | 1.8 | 85974 Kidney | 2.0 | 93582_Macrophages_LPS 100 | 1.8 |
| | | NAT | | ng/ml | |
| | | (OD04450-03) | | | |
| Kidney (fetal) | 1.9 | Kidney Cancer | 7.0 | 93098_HUVEC | 2.3 |
| | | Clontech | | (Endothelial)_none | |
| | | 8120607 | | | |
| Renal ca. 786-0 | 7.0 | Kidney NAT | 1.5 | 93099_HUVEC | 9.0 |
| | | Clontech | | (Endothelial)_starved | |
| | | 8120608 | | | |
| Renal ca. A498 | 6.8 | Kidney Cancer | 2.0 | 93100_HUVEC (Endothelial)_IL- | 1.2 |
| | | Clontech | | 1b | |
| | | 8120613 | | | |
| Renal ca.RXF | 4.7 | Kidney NAT | 4.1 | 93779_HUVEC (Endothelial)_IFN | 1.4 |
| 393 | | Clontech | | gamma | |
| | | 8120614 | | | |
| Renal ca.ACHN | 9.8 | Kidney Cancer | 2.2 | 93102_HUVEC | 0.8 |
| | | Clontech | | (Endothelial)_TNF alpha + IFN | |
| | | 9010320 | | gamma | |
| Renal ca.UO-31 | 1.3 | Kidney NAT | 3.5 | 93101_HUVEC | 1.1 |
| | | Clontech | | (Endothelial)_TNF alpha + IL4 | |
| | | 9010321 | | | |
| Renal ca.TK-10 | 0.6 | Normal Uterus | 3.1 | 93781_HUVEC (Endothelial)_IL- | 3.0 |
| | | GENPAK | | 11 | |
| | | 061018 | | | |
| Liver | 0.8 | Uterus Cancer | 17.6 | 93583_Lung Microvascular | 0.8 |
| | | GENPAK | | Endothelial Cells_none | |
| | | 064011 | | | |
| Liver (fetal) | 1.1 | Normal Thyroid | 3.7 | 93584_Lung Microvascular | 0.5 |
| | | Clontech A+ | | Endothelial Cells_TNFa (4 ng/ml) | |
| | | 6570-1 | | and IL1b (1 ng/ml) | |
| Liver ca. | 54.0 | Thyroid Cancer | 1.2 | 92662_Microvascular Dermal | 1.1 |
| (hepatoblast) | | GENPAK | | endothelium_none | |
| HepG2 | | 064010 | | | |
| Lung | 3.9 | Thyroid Cancer | 0.6 | 92663_Microsvasular Dermal | 1.0 |
| | | INVITROGEN | | endothelium_TNFa (4 ng/ml) and | |
| | | A302152 | | IL1b (1 ng/ml) | |
| Lung (fetal) | 9.0 | Thyroid NAT | 2.6 | 93773_Bronchial | 0.0 |
| | | INVITROGEN | | epithelium_TNFa (4 ng/ml) and | |
| | | A302153 | | IL1b (1 ng/ml)** | |
| Lung ca. (small | 34.4 | Normal Breast | 3.4 | 93347_Small Airway | 0.4 |
| cell) LX-1 | | GENPAK | | Epithelium_none | |
| | | 061019 | | | |
| Lung ca. (small | 3.0 | 84877 Breast | 0.9 | 93348_Small Airway | 0.5 |
| cell) NCI-H69 | | Cancer | | Epithelium_TNFa (4 ng/ml) and | |
| | | (OD04566) | | IL1b (1 ng/ml) | |
| Lung ca. (s.cell | 13.0 | 85975 Breast | 67.8 | 92668_Coronery Artery | 5.8 |
| var.) SHP-77 | | Cancer | | SMC_resting | |
| | | (OD04590-01) | | | |
| Lung ca. (large | 6.8 | 85976 Breast | 51.1 | 92669_Coronery Artery | 2.3 |
| cell)NCI-H460 | | Cancer Mets | | SMC_TNFa (4 ng/ml) and IL1b (1 | |
| | | (OD04590-03) | | ng/ml) | |
| Lung ca. (non- | 3.4 | 87070 Breast | 12.7 | 93107_astrocytes_resting | 2.7 |
| sm. cell) A549 | | Cancer | | | |
| | | Metastasis | | | |
| | | (OD04655-05) | | | |
| Lung ca. (non- | 34.4 | GENPAK Breast | 8.9 | 93108_astrocytes_TNFa (4 | 0.0 |
| s.cell) NCI-H23 | | Cancer 064006 | | ng/ml) and IL1b (1 ng/ml) | |
| Lung ca (non- | 10.5 | Breast Cancer | 6.2 | 92666_KU-812 (Basophil)_resting | 6.8 |
| s.cell) HOP-62 | | Clontech | | | |
| | | 9100266 | | | |
| Lung ca. (non- | 47.6 | Breast NAT | 3.3 | 92667_KU-812 | 8.4 |
| s.cl) NCI-H522 | | Clontech | | (Basophil)_PMA/ionoycin | |
| | | 9100265 | | | |
| Lung Ca. | 4.7 | Breast Cancer | 3.4 | 93579_CCD1106 | 1.6 |
| (squam.) SW | | INVITROGEN | | (Keratinocytes)_none | |
| 900 | | A209073 | | | |
| Lung Ca. | 0.7 | Breast NAT | 8.7 | 93580_CCD1106 | 1.4 |
| (squam.) NCI- | | INVITROGEN | | (Keratinocytes)_TNFa and IFNg** | |
| H596 | | A2090734 | | | |
| Mammary gland | 9.9 | Normal Liver | 1.1 | 93791_Liver Cirrhosis | 4.2 |
| | | GENPAK | | | |
| | | 061009 | | | |
| Breast ca.* (pl. | 5.6 | Liver Cancer | 0.6 | 93792_Lupus Kidney | 1.9 |
| effusion) MCF-7 | | GENPAK | | | |
| | | 064003 | | | |
| Breast ca.* (pl.ef) | 21.3 | Liver Cancer | 0.6 | 93577_NCI-H292 | 39.5 |
| MDA-MB-231 | | Research | | | |
| | | Genetics RNA | | | |
| | | 1025 | | | |
| Breast ca.* (pl. | 66.0 | Liver Cancer | 1.4 | 93358_NCI-H292_IL-4 | 39.0 |
| effusion) T47D | | Research | | | |
| | | Genetics RNA | | | |
| | | 1026 | | | |
| Breast ca. BT- | 7.6 | Paired Liver | 1.3 | 93360_NCI-H292_IL-9 | 65.5 |
| 549 | | Cancer Tissue | | | |
| | | Research | | | |
| | | Genetics RNA | | | |
| | | 6004-T | | | |
| Breast ca.MDA-N | 18.7 | Paired Liver | 1.3 | 93359_NCI-H292_IL-13 | 37.1 |
| | | Tissue Research | | | |
| | | Genetics RNA | | | |
| | | 6004-N | | | |
| Ovary | 12.1 | Paired Liver | 1.1 | 93357_NCI-H292_IFN gamma | 31.9 |
| | | Cancer Tissue | | | |
| | | Research | | | |
| | | Genetics RNA | | | |
| | | 6005-T | | | |
| Ovarian | 3.5 | Paired Liver | 0.3 | 93777_HPAEC_- | 0.5 |
| ca.OVCAR-3 | | Tissue Research | | | |
| | | Genetics RNA | | | |
| | | 6005-N | | | |
| Ovarian | 4.0 | Normal Bladder | 5.9 | 93778_HPAEC_IL-1 beta/TNA | 1.2 |
| ca.OVCAR-4 | | GENPAK | | alpha | |
| | | 061001 | | | |
| Ovarian ca. | 9.1 | Bladder Cancer | 1.7 | 93254_Normal Human Lung | 42.3 |
| OVCAR-5 | | Research | | Fibroblast_none | |
| | | Genetics RNA | | | |
| | | 1023 | | | |
| Ovarian ca. | 12.7 | Bladder Cancer | 1.9 | 93253_Normal Human Lung | 17.8 |
| OVCAR-8 | | INVITROGEN | | Fibroblast_TNFa (4 ng/ml) and IL- | |
| | | A302173 | | 1b (1 ng/ml) | |
| Ovarian | 9.8 | 87071 Bladder | 2.0 | 93257_Normal Human Lung | 100.0 |
| ca.IGROV-1 | | Cancer | | Fibroblast_IL-4 | |
| | | (OD04718-01) | | | |
| Ovarian ca.* | 0.4 | 87072 Bladder | 3.3 | 93256_Normal Human Lung | 72.7 |
| (ascites) SK-OV- | | Normal Adjacent | | Fibroblast_IL-9 | |
| 3 | | (OD04718-03) | | | |
| Uterus | 6.9 | Normal Ovary | 2.2 | 93255_Normal Human Lung | 60.7 |
| | | Res. Gen. | | Fibroblast_IL-13 | |
| Plancenta | 4.6 | Ovarian Cancer | 29.1 | 93258_Normal Human Lung | 81.8 |
| | | GENPAK | | Fibroblast_IFN gamma | |
| | | 064008 | | | |
| Prostate | 15.7 | 87492 Ovary | 100.0 | 93106_Dermal Fibroblasts | 76.8 |
| | | Cancer | | CCD1070_resting | |
| | | (OD04768-07) | | | |
| Prostate ca.* | 35.9 | 87493 Ovary | 2.2 | 93361_Dermal Fibroblasts | 30.2 |
| (bone met) PC-3 | | NAT (OD04768- | | CCD1070_TNF alpha 4 ng/ml | |
| | | 08) | | | |
| Testis | 14.6 | Normal Stomach | 13.1 | 93105_Dermal Fibroblasts | 38.2 |
| | | GENPAK | | CCD1070_IL-1 beta 1 ng/ml | |
| | | 061017 | | | |
| Melanoma | 13.5 | NAT Stomach | 8.8 | 93772_dermal fibroblast_IFN | 34.2 |
| Hs688(A).T | | Clontech | | gamma | |
| | | 9060359 | | | |
| Melanoma* (met) | 71.2 | Gastric Cancer | 2.5 | 93771_dermal fibroblast_IL-4 | 80.7 |
| Hs688(B).T | | Clontech | | | |
| | | 9060395 | | | |
| Melanoma | 1.7 | NAT Stomach | 9.7 | 93259_IBD Colitis 1** | 0.0 |
| UACC-62 | | Clontech | | | |
| | | 9060394 | | | |
| Melanoma M14 | 9.5 | Gastric Cancer | 15.9 | 93260_IBD Colitis 2 | 0.3 |
| | | Clontech | | | |
| | | 9060397 | | | |
| Melanoma LOX | 2.4 | NAT Stomach | 12.9 | 93261_IBD Crohns | 1.4 |
| IMVI | | Clontech | | | |
| | | 9060396 | | | |
| Melanoma* | 3.4 | Gastric Cancer | 12.1 | 735010_Colon_normal | 35.6 |
| (met)SK-MEL-5 | | GENPAK | | | |
| | | 064005 | | | |
| Adipose | 5.9 | | | 735019_Lung_none | 11.0 |
| | | | | 64028-1_Thymus_none | 5.8 |
| | | | | 64030-1_Kidney_none | 9.7 |

Taqman results shown in Table 9 demonstrates that cFCTR1 is highly expressed by tumor cell lines and also overexpressed in tumor tissues, specifically breast and ovarian tumor compared to Normal Adjacent Tissues (NAT). There are reports that follistatin can act as a modulator of tumor growth and its expression also correlate with polycystic ovary syndrome, a benign form of ovarian tumor.

TABLE 10
Primer Design for Probe Ag2773 (FCTR4)

| | | | | Start | SEQ ID |
|---|---|---|---|---|---|
| Primer | Sequences | TM | Length | Pos | NO |
| Forward | 5′-CCTTGCTTTGTCATATGCTGTT-3′ | 59.3 | 22 | 243 | 29 |
| Probe | FAM-5′-CCCTTTGCCTGGAATATAAACTCTCA-3′-TAMRA | 64.6 | 26 | 265 | 30 |
| Reverse | 5′-AGAGGAAGCTTTCTGGAGAAGA-3′ | 58.9 | 22 | 313 | 31 |

TABLE 11
TAQMAN RESULTS FOR CLONE FCTR4

| | Panel | | Panel | | Panel |
|---|---|---|---|---|---|
| Tissue_Name | 1D | Tissue_Name | 2D | Tissue_Name | 4D |
| Liver | 18.3 | Normal Colon | 41.2 | 93768_Secondary Th1_anti- | 12.7 |
| adenocarcinoma | | GENPAK 061003 | | CD28/anti-CD3 | |
| Heart (fetal) | 4.3 | 83219 CC Well to | 5.2 | 93769_Secondary Th2_anti- | 14.2 |
| | | Mod Diff | | CD28/anti-CD3 | |
| | | (ODO3866) | | | |
| Pancreas | 3.1 | 83220 CC NAT | 2.5 | 93770_Secondary Tr1_anti- | 14.7 |
| | | (ODO3866) | | CD28/anti-CD3 | |
| Pancreatic | 20.0 | 83221 CC Gr.2 | 0.7 | 93573_Secondary Th1_resting day 4- | 4.7 |
| ca.CAPAN 2 | | rectosigmoid | | 6 in IL-2 | |
| | | (ODO3868) | | | |
| Adrenal gland | 7.4 | 83222 CC NAT | 1.4 | 93572_Secondary Th2_resting day 4- | 3.5 |
| | | (ODO3868) | | 6 in IL-2 | |
| Thyroid | 6.8 | 83235 CC Mod | 14.0 | 93571_Secondary Tr1_resting day 4- | 7.0 |
| | | Diff (ODO3920) | | 6 in IL-2 | |
| Salivary gland | 2.5 | 83236 CC NAT | 13.9 | 93568_primary Th1_anti-CD28/anti- | 22.4 |
| | | (ODO3920) | | CD3 | |
| Pituitary gland | 5.7 | 83237 CC Gr.2 | 16.2 | 93569_primary Th2_anti-CD28/anti- | 16.3 |
| | | ascend colon | | CD3 | |
| | | (ODO3921) | | | |
| Brain (fetal) | 14.4 | 83238 CC NAT | 5.2 | 93570_primary Tr1_anti-CD28/anti- | 21.8 |
| | | (ODO3921) | | CD3 | |
| Brain (whole) | 19.6 | 83241 CC from | 13.9 | 93565_primary Th1_resting dy 4-6 in | 30.2 |
| | | Partial | | IL-2 | |
| | | Hepatectomy | | | |
| | | (ODO4309) | | | |
| Brain | 3.7 | 83242 Liver NAT | 12.7 | 93566_primary Th2_resting dy 4-6 in | 14.4 |
| (amygdala) | | (ODO4309) | | IL-2 | |
| Brain | 2.1 | 87472 Colon | 3.4 | 93567_primary Tr1_resting dy 4-6 in | 7.4 |
| (cerebellum) | | mets to lung | | IL-2 | |
| | | (OD04451-01) | | | |
| Brain | 22.7 | 87473 Lung NAT | 1.5 | 93351_CD45RA CD4 | 7.6 |
| (hippocampus) | | (OD04451-02) | | lymphocyte_anti-CD28/anti-CD3 | |
| Brain (thalamus) | 7.4 | Normal Prostate | 1.0 | 93352_CD45RO CD4 | 11.1 |
| | | Clontech A+ | | lymphocyte_anti-CD28/anti-CD3 | |
| | | 6546-1 | | | |
| Cerebral Cortex | 47.3 | 84140 Prostate | 3.1 | 93251_CD8 Lymphocytes_anti- | 9.6 |
| | | Cancer | | CD28/anti-CD3 | |
| | | (OD04410) | | | |
| Spinal cord | 8.3 | 84141 Prostate | 10.6 | 93353_chronic CD8 Lymphocytes | 9.7 |
| | | NAT (OD04410) | | 2ry_resting dy 4-6 in IL-2 | |
| CNS Ca. | 19.9 | 87073 Prostate | 9.7 | 93574_chronic CD8 Lymphocytes | 6.2 |
| (glio/astro)U87- | | Cancer | | 2ry_activated CD3/CD28 | |
| MG | | (OD04720-01) | | | |
| CNS ca. | 57.0 | 87074 Prostate | 8.3 | 93354_CD4_none | 6.4 |
| (glio/astro) U- | | NAT (OD04720- | | | |
| 118-MG | | 02) | | | |
| CNS ca. (astro) | 10.0 | Normal Lung | 36.6 | 93252_Secondary Th1/Th2/Tr1_anti- | 9.3 |
| SW1783 | | GENPAK 061010 | | CD95 CH11 | |
| CNS ca.* | 44.8 | 83239 Lung Met | 11.7 | 93103_LAK cells_resting | 11.0 |
| (neuro; met)SK- | | to Muscle | | | |
| N-AS | | (ODO4286) | | | |
| CNS ca. (astro) | 37.4 | 83240 Muscle | 3.4 | 93788_LAK cells_IL-2 | 10.4 |
| SF-539 | | NAT (ODO4286) | | | |
| CNS ca. (astro) | 62.0 | 84136 Lung | 15.1 | 93787_LAK cells_IL-2 + IL-12 | 7.4 |
| SNB-75 | | Malignant Cancer | | | |
| | | (OD03126) | | | |
| CNS ca. (glio) | 24.8 | 84137 Lung NAT | 17.4 | 93789_LAK cells_IL-2 + IFN gamma | 11.6 |
| SNB-19 | | (OD03126) | | | |
| CNS ca. (glio) | 40.3 | 84871 Lung | 5.0 | 93790_LAK cells_IL-2 + IL-18 | 13.3 |
| U251 | | Cancer | | | |
| | | (OD04404) | | | |
| CNS ca. (glio) | 100.0 | 84872 Lung NAT | 6.3 | 93104_LAK cells_PMA/ionomycin | 4.8 |
| SF-295 | | (OD04404) | | and IL-18 | |
| Heart | 0.0 | 84875 Lung | 3.2 | 93578_NK Cells IL-2_resting | 6.2 |
| | | Cancer | | | |
| | | (OD04565) | | | |
| Skeletal muscle | 0.0 | 85950 Lung | 15.8 | 93109_Mixed Lymphocyte | 12.3 |
| | | Cancer | | Reaction_Two Way MLR | |
| | | (OD04237-01) | | | |
| Bone marrow | 33.7 | 85970 Lung NAT | 10.5 | 93110_Mixed Lymphocyte | 8.7 |
| | | (OD04237-02) | | Reaction_Two Way MLR | |
| Thymus | 12.4 | 83255 Ocular | 5.9 | 93111_Mixed Lymphocyte | 3.5 |
| | | Mel Met to Liver | | Reaction_Two Way MLR | |
| | | (ODO4310) | | | |
| Spleen | 21.3 | 83256 Liver NAT | 3.6 | 93112_Mononuclear Cells | 4.5 |
| | | (ODO4310) | | (PBMCs)_resting | |
| Lymph node | 13.4 | 84139 Melanoma | 10.6 | 93113_Mononuclear Cells | 21.2 |
| | | Mets to Lung | | (PBMCs)_PWM | |
| | | (OD04321) | | | |
| Colorectal | 38.2 | 84138 Lung NAT | 10.6 | 93114_Mononuclear Cells | 8.9 |
| | | (OD04321) | | (PBMCs)_PHA-L | |
| Stomach | 9.9 | Normal Kidney | 26.2 | 93249_Ramos (B cell)_none | 100.0 |
| | | GENPAK 061008 | | | |
| Small intestine | 17.9 | 83786 Kidney | 22.2 | 93250_Ramos (B cell)_ionomycin | 28.7 |
| | | Ca, Nuclear | | | |
| | | grade 2 | | | |
| | | (OD04338) | | | |
| Colon | 27.7 | 83787 Kidney | 11.7 | 93349_B lymphocytes_PWM | 20.0 |
| ca.SW480 | | NAT (OD04338) | | | |
| Colon ca.* | 30.8 | 83788 Kidney Ca | 45.1 | 93350_B lymphoytes_CD40L and IL- | 7.8 |
| (SW480 | | Nuclear grade | | 4 | |
| met)SW620 | | 1/2 (OD04339) | | | |
| Colon ca.HT29 | 8.1 | 83789 Kidney | 14.8 | 92665_EOL-1 (Eosinophil)_dbcAMP | 8.0 |
| | | NAT (OD04339) | | differentiated | |
| Colon ca.HCT- | 35.4 | 83790 Kidney | 26.6 | 93248_EOL-1 | 3.8 |
| 116 | | Ca, Clear cell | | (Eosinophil)_dbcAMP/PMAionomycin | |
| | | type (ODO4340) | | | |
| Colon Ca. CaCo- | 37.6 | 83791 Kidney | 10.4 | 93356_Dendritic Cells_none | 6.8 |
| 2 | | NAT (OD04340) | | | |
| 83219 CC Well | 17.8 | 83792 Kidney | 2.4 | 93355_Dendritic Cells_LPS 100 ng/ml | 3.3 |
| to Mod Diff | | Ca, Nuclear | | | |
| (ODO3866) | | grade 3 | | | |
| | | (OD04348) | | | |
| Colon ca.HCC- | 19.9 | 83793 Kidney | 18.8 | 93775_Dendritic Cells_anti-CD40 | 6.3 |
| 2998 | | NAT (OD04348) | | | |
| Gastric ca.* | 73.2 | 87474 Kidney | 5.6 | 93774_Monocytes_resting | 10.6 |
| (liver met) NCI- | | Cancer | | | |
| N87 | | (OD04622-01) | | | |
| Bladder | 43.2 | 87475 Kidney | 0.5 | 93776_Monocytes_LPS 50 ng/ml | 3.5 |
| | | NAT (OD04622- | | | |
| | | 03) | | | |
| Trachea | 10.3 | 85973 Kidney | 21.2 | 93581_Macrophages_resting | 7.6 |
| | | Cancer | | | |
| | | (OD04450-01) | | | |
| Kidney | 9.2 | 85974 Kidney | 9.3 | 93582_Macrophages_LPS 100 ng/ml | 3.9 |
| | | NAT (OD04450- | | | |
| | | 03) | | | |
| Kidney (fetal) | 0.0 | Kidney Cancer | 0.0 | 93098_HUVEC (Endothelial)_none | 8.5 |
| | | Clontech | | | |
| | | 8120607 | | | |
| Renal ca.786-0 | 53.6 | Kidney NAT | 0.9 | 93099_HUVEC (Endothelial)_starved | 17.9 |
| | | Clontech | | | |
| | | 8120608 | | | |
| Renal ca.A498 | 36.1 | Kidney Cancer | 0.0 | 93100_HUVEC (Endothelial)_IL-1b | 6.0 |
| | | Clontech | | | |
| | | 8120613 | | | |
| Renal ca.RXF | 31.6 | Kidney NAT | 0.9 | 93779_HUVEC (Endothelial)_IFN | 7.8 |
| 393 | | Clontech | | gamma | |
| | | 8120614 | | | |
| Renal ca.ACHN | 21.6 | Kidney Cancer | 2.7 | 93102_HUVEC (Endothelial)_TNF | 5.7 |
| | | Clontech | | alpha + IFN gamma | |
| | | 9010320 | | | |
| Renal ca.UO-31 | 28.7 | Kidney NAT | 5.0 | 93101_HUVEC (Endothelial)_TNF | 5.6 |
| | | Clontech | | alpha + IL4 | |
| | | 9010321 | | | |
| Renal ca.TK-10 | 7.0 | Normal Uterus | 5.3 | 93781_HUVEC (Endothelial)_IL-11 | 4.9 |
| | | GENPAK 061018 | | | |
| Liver | 14.2 | Uterus Cancer | 9.0 | 93583_Lung Microvascular | 4.9 |
| | | GENPAK 064011 | | Endothelial Cells_none | |
| Liver (fetal) | 14.5 | Normal Thyroid | 3.4 | 93584_Lung Microvascular | 4.9 |
| | | Clontech A+ | | Endothelial Cells_TNFa (4 ng/ml) and | |
| | | 6570-1 | | IL1b (1 ng/ml) | |
| Liver ca. | 59.9 | Thyroid Cancer | 1.8 | 92662_Microvascular Dermal | 8.6 |
| (hepatoblast) | | GENPAK 064010 | | endothelium_none | |
| HepG2 | | | | | |
| Lung | 17.8 | Thyroid Cancer | 3.6 | 92663_Microsvasular Dermal | 6.0 |
| | | INVITROGEN | | endothelium_TNFa (4 ng/ml) and IL1b | |
| | | A302152 | | (1 ng/ml) | |
| Lung (fetal) | 9.6 | Thyroid NAT | 4.9 | 93773_Bronchial epithelium_TNFa (4 | 0.9 |
| | | INVITROGEN | | ng/ml) and IL1b (1 ng/ml)** | |
| | | A302153 | | | |
| Lung ca. (small | 70.2 | Normal Breast | 8.5 | 93347_Small Airway Epithelium_none | 1.3 |
| cell) LX-1 | | GENPAK 061019 | | | |
| Lung ca. (small | 29.9 | 84877 Breast | 1.5 | 93348_Small Airway | 13.2 |
| cell) NCI-H69 | | Cancer | | Epithelium_TNFa (4 ng/ml) and IL1b | |
| | | (OD04566) | | (1 ng/ml) | |
| Lung ca. (s.cell | 3.9 | 85975 Breast | 23.8 | 92668_Coronery Artery SMC_resting | 3.4 |
| var.) SHP-77 | | Cancer | | | |
| | | (OD04590-01) | | | |
| Lung ca. (large | 2.0 | 85976 Breast | 24.5 | 92669_Coronery Artery SMC_TNFa | 2.0 |
| cell) NCI-H460 | | Cancer Mets | | (4 ng/ml) and IL1b (1 ng/ml) | |
| | | (OD04590-03) | | | |
| Lung ca. (non- | 28.5 | 87070 Breast | 12.9 | 93107_astrocytes_resting | 4.7 |
| sm. cell) A549 | | Cancer | | | |
| | | Metastasis | | | |
| | | (OD04655-05) | | | |
| Lung ca. (non- | 36.1 | GENPAK Breast | 11.8 | 93108_astrocytes_TNFa (4 ng/ml) | 1.9 |
| s.cell) NCI-H23 | | Cancer 064006 | | and IL1b (1 ng/ml) | |
| Lung ca (non- | 29.9 | Breast Cancer | 3.2 | 92666_KU-812 (Basophil)_resting | 5.8 |
| s.cell HOP-62 | | Clontech | | | |
| | | 9100266 | | | |
| Lung ca. (non- | 17.2 | Breast NAT | 1.8 | 92667_KU-812 | 12.0 |
| s.cl) NCI-H522 | | Clontech | | (Basophil)_PMA/ionoycin | |
| | | 9100265 | | | |
| Lung ca. | 63.7 | Breast Cancer | 11.0 | 93579_CCD1106 | 4.9 |
| (squam.) SW | | INVITROGEN | | (Keratinocytes)_none | |
| 900 | | A209073 | | | |
| Lung ca. | 10.0 | Breast NAT | 7.1 | 93580_CCD1106 | 0.3 |
| (squam.) NCI- | | INVITROGEN | | (Keratinocytes)_TNFa and IFNg** | |
| H596 | | A2090734 | | | |
| Mammary gland | 4.6 | Normal Liver | 8.8 | 93791_Liver Cirrhosis | 1.8 |
| | | GENPAK 061009 | | | |
| Breast ca.* (pl. | 0.0 | Liver Cancer | 4.9 | 93792_Lupus Kidney | 1.6 |
| effusion) MCF- | | GENPAK 064003 | | | |
| 7 | | | | | |
| Breast ca.* | 38.7 | Liver Cancer | 1.0 | 93577_NCI-H292 | 11.1 |
| (pl.ef) MDA-MB- | | Research | | | |
| 231 | | Genetics RNA | | | |
| | | 1025 | | | |
| Breast ca.* (pl. | 0.0 | Liver Cancer | 0.8 | 93358_NCI-H292_IL-4 | 12.2 |
| effusion) T47D | | Research | | | |
| | | Genetics RNA | | | |
| | | 1026 | | | |
| Breast ca.BT- | 4.6 | Paired Liver | 3.0 | 93360_NCI-H292_IL-9 | 7.6 |
| 549 | | Cancer Tissue | | | |
| | | Research | | | |
| | | Genetics RNA | | | |
| | | 6004-T | | | |
| Breast ca.MDA- | 19.0 | Paired Liver | 7.3 | 93359_NCI-H292_IL-13 | 6.1 |
| N | | Tissue Research | | | |
| | | Genetics RNA | | | |
| | | 6004-N | | | |
| Ovary | 1.7 | Paired Liver | 0.2 | 93357_NCI-H292_IFN gamma | 5.8 |
| | | Cancer Tissue | | | |
| | | Research | | | |
| | | Genetics RNA | | | |
| | | 6005-T | | | |
| Ovarian | 4.8 | Paired Liver | 0.0 | 93777_HPAEC_- | 6.8 |
| ca.OVCAR-3 | | Tissue Research | | | |
| | | Genetics RNA | | | |
| | | 6005-N | | | |
| Ovarian | 0.0 | Normal Bladder | 19.8 | 93778_HPAEC_IL-1 beta/TNA alpha | 5.4 |
| ca.OVCAR-4 | | GENPAK 061001 | | | |
| Ovarian | 39.0 | Bladder Cancer | 3.1 | 93254_Normal Human Lung | 2.1 |
| ca.OVCAR-5 | | Research | | Fibroblast_none | |
| | | Genetics RNA | | | |
| | | 1023 | | | |
| Ovarian | 36.6 | Bladder Cancer | 9.9 | 93253_Normal Human Lung | 1.9 |
| ca.OVCAR-8 | | INVITROGEN | | Fibroblast_TNFa (4 ng/ml) and IL-1b | |
| | | A302173 | | (1 ng/ml) | |
| Ovarian | 0.0 | 87071 Bladder | 6.6 | 93257_Normal Human Lung | 3.6 |
| ca.IGROV-1 | | Cancer | | Fibroblast_IL-4 | |
| | | (OD04718-01) | | | |
| Ovarian ca.* | 65.5 | 87072 Bladder | 4.0 | 93256_Normal Human Lung | 3.3 |
| (ascites) SK- | | Normal Adjacent | | Fibroblast IL-9 | |
| OV-3 | | (OD04718-03) | | | |
| Uterus | 1.6 | Normal Ovary | 0.3 | 93255_Normal Human Lung | 2.3 |
| | | Res. Gen. | | Fibroblast_IL-13 | |
| Plancenta | 8.9 | Ovarian Cancer | 6.8 | 93258_Normal Human Lung | 2.9 |
| | | GENPAK 064008 | | Fibroblast_IFN gamma | |
| Prostate | 0.0 | 87492 Ovary | 100.0 | 93106_Dermal Fibroblasts | 5.6 |
| | | Cancer | | CCD1070_resting | |
| | | (OD04768-07) | | | |
| Prostate ca.* | 9.2 | 87493 Ovary | 3.6 | 93361_Dermal Fibroblasts | 17.4 |
| (bone met)PC-3 | | NAT (OD04768- | | CCD1070_TNF alpha 4 ng/ml | |
| | | 08) | | | |
| Testis | 29.5 | Normal Stomach | 8.6 | 93105_Dermal Fibroblasts | 3.8 |
| | | GENPAK 061017 | | CCD1070_IL-1 beta 1 ng/ml | |
| Melanoma | 14.3 | NAT Stomach | 0.7 | 93772_dermal fibroblast_IFN gamma | 2.6 |
| Hs688(A).T | | Clontech | | | |
| | | 9060359 | | | |
| Melanoma* | 22.9 | Gastric Cancer | 3.9 | 93771_dermal fibroblast_IL-4 | 3.4 |
| (met) | | Clontech | | | |
| Hs688(B).T | | 9060395 | | | |
| Melanoma | 9.7 | NAT Stomach | 5.3 | 93259_IBD Colitis 1** | 0.2 |
| UACC-62 | | Clontech | | | |
| | | 9060394 | | | |
| Melanoma M14 | 12.7 | Gastric Cancer | 13.2 | 93260_IBD Colitis 2 | 0.4 |
| | | Clontech | | | |
| | | 9060397 | | | |
| Melanoma LOX | 4.5 | NAT Stomach | 1.1 | 93261_IBD Crohns | 0.3 |
| IMVI | | Clontech | | | |
| | | 9060396 | | | |
| Melanoma* | 21.8 | Gastric Cancer | 23.0 | 735010_Colon_normal | 3.3 |
| (met) SK-MEL-5 | | GENPAK 064005 | | | |
| Adipose | 6.7 | | | 735019_Lung_none | 3.9 |
| | | | | 64028-1_Thymus_none | 7.7 |
| | | | | 64030-1_Kidney_none | 21.8 |

Table 12 shows the taqman results of clone FCTR4 indicating overexpression in ovarian cancer as compared to Normal Adjacent Tissue AT). In addition, increased expression is demonstrated by ovarian tumor cell line suggesting that antibodies could be used to treat ovarian tumors.

TABLE 13
Primer Design for Probe Ag427 (FCTR5)

| | | | Start | SEQ ID |
|---|---|---|---|---|
| Primer | Sequences | Length | Pos | NO |
| Forward | 5′-GAGCTACAGGCAGCCTCGAGT-3′ | 21 | 443 | 32 |
| Probe | TET-5′-TGGCCCAGCTGACCCTGCTCA-3′-TAMPA | 21 | | 33 |
| Reverse | 5′-GGCTACGTCAGTGGGTfTGG-3′ | 20 | 449 | 34 |

TABLE 14
Taqman results for FCTR5

| Tissue_Name | Panel 1 | Tissue_Name | Panel 4D |
|---|---|---|---|
| Endothelial cells | 10.7 | 93768_Secondary Th1_anti-CD28/anti-CD3 | 15.9 |
| Endothelial cells (treated) | 15.2 | 93769_Secondary Th2_anti-CD28/anti-CD3 | 14.7 |
| Pancreas | 16.2 | 93770_Secondary Tr1_anti-CD28/anti-CD3 | 21.9 |
| Pancreatic ca.CAPAN 2 | 10.5 | 93573_Secondary Th1_resting day 4-6 in IL-2 | 12.3 |
| Adipose | 45.1 | 93572_Secondary Th2_resting day 4-6 in IL-2 | 16.2 |
| Adrenal gland | 61.6 | 93571_Secondary Tr1_resting day 4-6 in IL-2 | 16.2 |
| Thyroid | 13.1 | 93568_primary Th1_anti-CD28/anti-CD3 | 13.9 |
| Salavary gland | 33.7 | 93569_primary Th2_anti-CD28/anti-CD3 | 14.6 |
| Pituitary gland | 15.8 | 93570_primary Tr1_anti-CD28/anti-CD3 | 26.2 |
| Brain (fetal) | 7.2 | 93565_primary Th1_resting dy 4-6 in IL-2 | 56.3 |
| Brain (whole) | 6.3 | 93566_primary Th2_resting dy 4-6 in IL-2 | 27.7 |
| Brain (amygdala) | 8.4 | 93567_primary Tr1_resting dy 4-6 in IL-2 | 31.6 |
| Brain (cerebellum) | 6.8 | 93351_CD45RA CD4 lymphocyte_anti- | 12.1 |
| | | CD28/anti-CD3 | |
| Brain (hippocampus) | 7.9 | 93352_CD45RO CD4 lymphocyte_anti- | 17.1 |
| | | CD28/anti-CD3 | |
| Brain substantia nigra | 9.5 | 93251_CD8 Lymphocytes_anti-CD28/anti-CD3 | 9.1 |
| Brain (thalamus) | 7.9 | 93353_chronic CD8 Lymphocytes | 13.4 |
| | | 2ry_resting dy 4-6 in IL-2 | |
| Brain (hypothalamus) | 23.0 | 93574_chronic CD8 Lymphocytes | 9.2 |
| | | 2ry_activated CD3/CD28 | |
| Spinal cord | 9.5 | 93354_CD4_none | 7.6 |
| CNS ca. (glio/astro)U87-MG | 12.6 | 93252_Secondary Th1/Th2/Tr1_anti-CD95 CH11 | 20.2 |
| CNS ca. (glio/astro)U- | 11.6 | 93103_LAK cells_resting | 57.0 |
| 118-MG | | | |
| CNS ca. (astro)SW1783 | 4.3 | 93788_LAK cells_IL-2 | 18.8 |
| CNS ca.* (neuro; met )SK-N- | 10.4 | 93787_LAK cells_IL2 + IL-12 | 14.2 |
| AS | | | |
| CNS ca. (astro) SF-539 | 11.6 | 93789_LAK cells_IL-2 + IFN gamma | 20.9 |
| CNS ca. (astro) SNB-75 | 4.4 | 93790_LAK cells_IL-2 + IL-18 | 14.8 |
| CNS ca. (glio)SNB-19 | 31.6 | 93104 LAK_cells_PMA/ionomycin and IL-18 | 12.9 |
| CNS ca. (glio)U251 | 17.3 | 93578_NK Cells IL-2_resting | 17.4 |
| CNS ca. (glio)SF-295 | 20.9 | 93109_Mixed Lymphocyte Reaction_Two | 43.5 |
| | | Way MLR | |
| Heart | 14.3 | 93110_Mixed Lymphocyte Reaction_Two | 19.3 |
| | | WayMLR | |
| Skeletal muscle | 11.7 | 93111_Mixed Lymphocyte Reaction_Two | 12.6 |
| | | Way MLR | |
| Bone marrow | 21.9 | 93112_Mononuclear Cells (PBMCs)_resting | 8.7 |
| Thymus | 20.9 | 93113_Mononuclear Cells (PBMCs)_PWM | 28.5 |
| Spleen | 23.8 | 93114_Mononuclear Cells (PBMCs)_PHA-L | 26.2 |
| Lymph node | 24.2 | 93249_Ramos (B cell)_none | 0.3 |
| Colon (ascending) | 17.2 | 93250_Ramos (B cell)_ionomycin | 1.2 |
| Stomach | 11.1 | 93349_B lymphocytes_PWM | 25.7 |
| Small intestine | 21.5 | 93350_B lymphoytes_CD40L and IL-4 | 13.0 |
| Colon ca.SW480 | 12.2 | 92665_EOL-1 (Eosinophil)_dbcAMP | 26.4 |
| | | differentiated | |
| Colon ca.* (SW480 | 8.6 | 93248_EOL-1 | 11.4 |
| met)SW620 | | (Eosinophil)....dbcAMP/PMAionomycin | |
| Colon ca.HT29 | 16.2 | 93356_Dendritic Cells_none | 40.3 |
| Colon ca.HCT-116 | 8.1 | 93355_Dendritic Cells_LPS 100 ng/ml | 33.0 |
| Colon ca.CaCo-2 | 22.1 | 93775_Dendritic Cells_anti-CD40 | 20.5 |
| Colon ca.HCT-15 | 18.6 | 93774_Monocytes_resting | 23.3 |
| Colon ca.HCC-2998 | 21.9 | 93776_Monocytes_LPS 50 ng/ml | 6.9 |
| Gastric ca.* (liver met) NCI- | 42.9 | 93581_Macrophages_resting | 14.7 |
| N87 | | | |
| Bladder | 95.3 | 93582_Macrophages_LPS 100 ng/ml | 64.6 |
| Trachea | 18.3 | 93098_HUVEC (Endothelial)_none | 6.8 |
| Kidney | 25.7 | 93099_HUVEC (Endothelial)_starved | 13.9 |
| Kidney (fetal) | 15.8 | 93100_HUVEC (Endothelial)_IL-1b | 7.5 |
| Renal ca.786-0 | 16.5 | 93779_HUVEC (Endothelial)_IFN gamma | 27.7 |
| Renal ca.A498 | 16.5 | 93102_HUVEC (Endothelial)_TNF alpha + | 11.8 |
| | | IFN gamma | |
| Renal ca.RXF 393 | 7.4 | 93101_HUVEC (Endothelial)_TNF alpha + | 6.7 |
| | | IL4 | |
| Renal ca.ACHN | 11.9 | 93781_HUVEC (Endothelial)_IL-11 | 10.4 |
| Renal ca.UO-31 | 15.8 | 93583_Lung Microvascular Endothelial | 8.8 |
| | | Cells_none | |
| Renal ca.TK-10 | 28.7 | 93584_Lung Microvascular Endothelial | 8.6 |
| | | Cells_TNFa (4 ng/ml) and IL1b (1 | |
| | | nglml) | |
| Liver | 100.0 | 92662_Microvascular Dermal | 22.1 |
| | | endothelium_none | |
| Liver (fetal) | 81.8 | 92663_Microsvasular Dermal | |
| | | endothelium_TNFa (4 ng/ml) and IL1b | 18.7 |
| | | (1 ng/ml) | |
| Liver ca. (hepatoblast) HepG2 | 28.3 | 93773_Bronchial epithelium_TNFa (4 | 35.4 |
| ng/ml) and IL1b (1 ng/ml) ** | | | |
| Lung | 10.7 | 93347_Small Airway Epithelium_none | 10.9 |
| Lung (fetal) | 10.9 | 93348_Small Airway Epithelium_TNFa (4 | 50.0 |
| | | ng/ml) and IL1b (1 ng/ml) | |
| Lung Ca. (small cell) LX-1 | 24.3 | 92668_Coronery Artery SMC_resting | 27.9 |
| Lung ca. (small cell) | 41.5 | 92669_Coronery Artery SMC_TNFa (4 | 25.4 |
| NCI-H69 | | ng/ml) and ILIb (1 ng/ml) | |
| Lung ca. (s.cell var.) SHP-77 | 4.6 | 93107_astrocytes_resting | 7.4 |
| Lung ca. (larg cell)NCI-H460 | 46.3 | 93108_astrocytes_TNFa (4 ng/ml) and IL1b | 10.7 |
| | | (1 ng/ml) | |
| Lung ca. (non-sm. cell) A549 | 45.4 | 92666_KU-812 (Basophil)_resting | 3.2 |
| Lung ca. (non-s.cell) | 54.3 | 92667_KU-812 (Basophil)_PMA/ionoycin | 6.7 |
| NCI-H23 | | | |
| Lung ca (non-s.cell) HOP-62 | 50.7 | 93579_CCD1106 (Keratinocytes)_none | 12.2 |
| Lund ca. (non-s.cl) NCI-H522 | 38.4 | 93580_CCD1106 (Keratinocytes)_TNFa | 100.0 |
| | | and IFNg ** | |
| Lung ca. (squam.) SW 900 | 30.8 | 93791_Liver Cirrhosis | 27.6 |
| Lung ca. (squam.) NCI-H596 | 15.5 | 93792_Lupus Kidney | 32.3 |
| Mammary gland | 65.5 | 93577_NCI-H292 | 77.4 |
| Breast ca.* (pl. effusion) | 4.4 | 93358_NCI-H292_IL-4 | 70.2 |
| MCF-7 | | | |
| Breast ca.* (pl.ef) MDA-MB- | 3.5 | 93360_NCI-H292_IL-9 | 54.3 |
| 231 | | | |
| Breast ca.* (pl. effusion)T47D | 8.7 | 93359_NCI-H292_IL-13 | 47.0 |
| Breast ca. BT-549 | 5.7 | 93357_NCI-H292_IFN gamma | 52.9 |
| Breast ca.MDA-N | 16.6 | 93777_HPAEC_- | 23.8 |
| Ovary | 20.5 | 93778_HPAEC_IL-1 beta/TNA alpha | 21.5 |
| Ovarian ca.OVCAR-3 | 21.6 | 93254_Normal Human Lung Fibroblast_none | 49.3 |
| Ovarian ca.OVCAR-4 | 8.3 | 93253_Normal Human Lung | 40.3 |
| | | Fibroblast_TNFa (4 ng/ml) and IL-1b | |
| | | (1 ng/ml) | |
| Ovarian ca.OVCAR-5 | 26.1 | 93257_Normal Human Lung Fibroblast_IL-4 | 48.3 |
| Ovarian ca.OVCAR-8 | 48.0 | 93256_Normal Human Lung Fibroblast_IL-9 | 29.3 |
| Ovarian ca.IGROV-1 | 9.3 | 93255_Normal Human Lung Fibroblast_IL-13 | 73.7 |
| Ovarian ca.*(ascites)SK-OV-3 | 8.8 | 93258_Normal Human Lung Fibroblast_IFN | 66.9 |
| | | gamma | |
| Uterus | 13.4 | 93106_Dermal Fibroblasts | 20.2 |
| CCD1070_resting | | | |
| Plancenta | 9.4 | 93361_Dermal Fibroblasts CCD1070_TNF | 35.1 |
| | | alpha 4 ng/ml | |
| Prostate | 21.3 | 93105_Dermal Fibroblasts CCD1070_IL-1 | |
| | | beta 1 ng/ml | |
| Prostate ca.* (bone met)PC-3 | 17.7 | 93772_dermal fibroblast_IFN gamma | 21.8 |
| Testis | 11.7 | 93771_dermal fibroblast_IL-4 | 21.2 |
| Melanoma Hs688(A).T | 9.0 | 93259_IBD Colitis 1** | 8.8 |
| Melanoma* (met) Hs688(B).T | 12.9 | 93260_IBD Colitis 2 | 3.5 |
| Melanoma UACC-62 | 12.4 | 93261_IBD Crohns | 1.3 |
| Melanoma M14 | 9.5 | 735010_Colon_normal | 20.3 |
| Melanoma LOX IMVI | 8.1 | 735019_Lung_none | 40.3 |
| Melanoma* (met) SK-MEL-5 | 8.8 | 64028-1_Thymus_none | 33.5 |
| Melanoma SK-MEL-28 | 8.0 | 64030-1_Kidney_none | 21.0 |

Taqman results in Table 14 show high expression of clone FCTR5 in bladder, liver and adrenal gland suggesting a possible role in the treatment of diseases involving these tissues.

TABLE 15
Primer Design for Probe Ag1541 (FCTR6)

| | | | | | SEQ ID |
|---|---|---|---|---|---|
| Primer | Sequences | TM | Length | tart Pos. | NO |
| Forward | 5′-AGAAGAACACCCCAGGGATATA-3′ | 58.8 | 22 | 1076 | 35 |
| Probe | FAM-5′-CCTCGTTGGTGAACTACAACCTCTGG-3′-TAMRA | 67.9 | 26 | 1100 | 36 |
| Reverse | 5′-CCTCTAGCTGGGTCACTTTCTC-3′ | 59.5 | 22 | 1129 | 37 |

TABLE 16
TAQMAN RESULTS FOR FCTR6 (PANEL ID)

| | Panel ID | |
|---|---|---|
| Tissue_Name | Run 1 | Run 2 |
| Liver adenocarcinoma | 0.0 | 0.0 |
| Heart (fetal) | 0.0 | 0.0 |
| Pancreas | 0.0 | 0.0 |
| Pancreatic ca.CAPAN 2 | 0.0 | 0.0 |
| Adrenal gland | 0.0 | 0.0 |
| Thyroid | 0.0 | 0.0 |
| Salivary gland | 0.0 | 0.0 |
| Pituitary gland | 0.0 | 0.0 |
| Brain (fetal) | 0.5 | 0.4 |
| Brain (whole) | 1.1 | 1.7 |
| Brain (amygdala) | 0.0 | 1.8 |
| Brain (cerebellum) | 0.6 | 1.9 |
| Brain (hippocampus) | 3.3 | 3.4 |
| Brain (thalamus) | 1.0 | 1.2 |
| Cerebral Cortex | 1.6 | 2.6 |
| Spinal cord | 2.5 | 0.4 |
| CNS ca. (glio/astro)U87-MG | 0.0 | 0.0 |
| CNS ca. (glio/astro)U-118-MG | 0.0 | 0.0 |
| CNS ca. (astro)SW1783 | 0.0 | 0.0 |
| CNS ca.* (neuro; met )SK-N-AS | 0.0 | 0.0 |
| CNS ca. (astro)SF-539 | 0.0 | 0.0 |
| CNS ca. (astro) SNB-75 | 0.7 | 0.0 |
| CNS ca. (glio)SNB-19 | 0.0 | 0.0 |
| CNS ca. (glio)U251 | 0.0 | 0.0 |
| CNS ca. (glio)SF-295 | 0.0 | 0.8 |
| Heart | 0.0 | 0.0 |
| Skeletal muscle | 0.0 | 0.0 |
| Bone marrow | 0.0 | 0.0 |
| Thymus | 0.0 | 0.0 |
| Spleen | 0.0 | 0.0 |
| Lymph node | 0.0 | 0.0 |
| Colorectal | 0.0 | 0.6 |
| Stomach | 1.9 | 0.0 |
| Small intestine | 0.0 | 1.0 |
| Colon ca. SW480 | 0.0 | 0.0 |
| Colon ca.* (SW480 met)SW620 | 0.0 | 0.0 |
| Colon ca. HT29 | 0.0 | 0.0 |
| Colon ca. HCT-116 | 0.6 | 0.4 |
| Colon ca.CaCo-2 | 1.5 | 0.0 |
| 83219 CC Well to Mod Duff (ODO3866) | 0.0 | 0.0 |
| Colon ca.HCC-2998 | 0.0 | 0.0 |
| Gastric ca.* (liver met) NCI-N87 | 1.2 | 0.0 |
| Bladder | 0.0 | 0.0 |
| Trachea | 0.0 | 0.4 |
| Kidney | 0.8 | 1.2 |
| Kidney (fetal) | 0.5 | 0.7 |
| Renal ca.786-0 | 0.0 | 0.0 |
| Renal ca.A498 | 0.0 | 0.0 |
| Renal ca.RXF 393 | 0.0 | 0.0 |
| Renal ca.ACHN | 0.0 | 0.0 |
| Renal ca. UO-31 | 0.0 | 0.0 |
| Renal ca.TK-10 | 0.0 | 0.0 |
| Liver | 0.0 | 0.0 |
| Liver (fetal) | 0.2 | 0.0 |
| Liver ca. (hepatoblast) HepG2 | 0.0 | 0.0 |
| Lung | 0.0 | 0.0 |
| Lung (fetal) | 0.0 | 0.0 |
| Lung ca. (small cell) LX-1 | 1.7 | 2.3 |
| Lung ca. (small cell)NCI-H69 | 0.0 | 0.0 |
| Lung ca. (s.cell var.) SHP-77 | 1.3 | 2.5 |
| Lung ca. (large cell)NCI-H460 | 0.0 | 0.0 |
| Lung ca. (non-sm. cell) A549 | 0.0 | 0.0 |
| Lung ca. (non-s.cell) NCI-H23 | 1.2 | 0.4 |
| Lung ca (non-s.cell) HOP-62 | 0.0 | 0.0 |
| Lung ca. (non-s.cl) NCI-H522 | 0.0 | 0.0 |
| Lung ca. (squam.) SW 900 | 0.0 | 0.7 |
| Lung ca. (squam.) NCI-H596 | 0.0 | 1.3 |
| Mammary gland | 0.0 | 1.5 |
| Breast ca.* (pl. effusion) MCF-7 | 0.0 | 0.0 |
| Breast ca.* (pl.ef) MDA-MB-231 | 5.8 | 0.5 |
| Breast ca.* (pl. effusion) T47D | 1.2 | 0.3 |
| Breast ca. BT-549 | 0.5 | 0.0 |
| Breast ca. MDA-N | 0.0 | 0.0 |
| Ovary | 0.0 | 0.0 |
| Ovarian ca. OVCAR-3 | 0.0 | 0.0 |
| Ovarian ca.OVCAR-4 | 0.0 | 0.0 |
| Ovarian ca.OVCAR-5 | 3.6 | 0.7 |
| Ovarian ca.OVCAR-8 | 0.0 | 0.0 |
| Ovarian ca.IGROV-1 | 0.0 | 0.0 |
| Ovarian ca.* (ascites) SK-OV-3 | 0.0 | 0.0 |
| Uterus | 0.0 | 0.0 |
| Plancenta | 0.0 | 0.0 |
| Prostate | 0.0 | 0.7 |
| Prostate ca.* (bone met)PC-3 | 0.0 | 0.0 |
| Testis | 100.0 | 100.0 |
| Melanoma Hs688(A).T | 0.0 | 0.0 |
| Melanoma* (met) Hs688(B).T | 0.0 | 0.0 |
| Melanoma UACC-62 | 0.0 | 0.0 |
| Melanoma M14 | 0.0 | 0.0 |
| Melanoma LOX IMVI | 0.0 | 0.0 |
| Melanoma* (met)SK-MEL-5 | 0.0 | 0.0 |
| Adipose | 0.5 | 0.0 |

TABLE 17
Taqman Results for FCTR6 (Panel 2D)

| | Panel 2D | |
|---|---|---|
| Tissue_Name | Run 1 | Run 2 |
| Normal Colon GENPAK 061003 | 5.4 | 2.4 |
| 83219 CC Well to Mod Duff (ODO3866) | 7.3 | 0.0 |
| 83220 CC NAT (ODO3866) | 5.8 | 1.5 |
| 83221 CC Gr.2 rectosigmoid (ODO3868) | 3.4 | 0.0 |
| 83222 CC NAT (ODO3868) | 0.0 | 0.0 |
| 83235 CC Mod Duff (ODO3920) | 11.0 | 1.4 |
| 83236 CC NAT (ODO3920) | 0.0 | 0.0 |
| 83237 CC Gr.2 ascend colon (ODO3921) | 6.2 | 2.5 |
| 83238 CC NAT (ODO3921) | 10.2 | 0.0 |
| 83241 CC from Partial Hepatectom (ODO4309) | 3.6 | 0.0 |
| 83242 Liver NAT (ODO4309) | 0.0 | 2.4 |
| 87472 Colon mets to lung (ODO4451-01) 7.2 | 4.4 | |
| 87473 Lung NAT (ODO4451-02) | 0.0 | 0.0 |
| Normal Prostate Clontech A+ 6546-1 | 4.8 | 2.9 |
| 84140 Prostate Cancer (ODO4410) | 3.5 | 0.0 |
| 84141 Prostate NAT (ODO4410) | 3.4 | 0.0 |
| 87073 Prostate Cancer (ODO4720-01) | 9.0 | 8.5 |
| 87074 Prostate NAT (ODO4720-02) | 0.0 | 0.0 |
| Normal Lung GENPAK 061010 | 17.7 | 6.5 |
| 83239 Lung Met to Muscle (ODO4286) | 0.0 | 2.3 |
| 83240 Muscle NAT (ODO4286) | 0.0 | 0.0 |
| 84136 Lung Malignant Cancer (ODO3126) 6.5 | 5.7 | |
| 84137 Lung NAT (ODO3126) | 0.0 | 0.0 |
| 84871 Lung Cancer (ODO4404) | 0.0 | 0.0 |
| 84872 Lung NAT (ODO4404) | 0.0 | 0.0 |
| 84875 Lung Cancer (ODO4565) | 0.0 | 0.0 |
| 85950 Lung Cancer (ODO4237-01) | 0.0 | 0.0 |
| 85970 Lung NAT (ODO4237-02) | 0.0 | 0.0 |
| 83255 Ocular Mel Met to Liver (ODO4310) | 4.3 | 0.0 |
| 83256 Liver NAT (ODO4310) | 0.0 | 0.0 |
| 84139 Melanoma Mets to Lung (ODO4321) 0.0 | 0.0 | |
| 84138 Lung NAT (ODO4321) | 0.0 | 0.0 |
| Normal Kidney GENPAK 061008 | 28.1 | 39.2 |
| 83786 Kidney Ca, Nuclear grade 2 (ODO4338) | 0.0 | 3.0 |
| 83787 Kidney NAT (ODO4338) | 22.7 | 31.6 |
| 83788 Kidney Ca Nuclear grade ½ (ODO4339) 0.0 | 3.1 | |
| 83789 Kidney NAT (ODO4339) | 97.3 | 100.0 |
| 83790 Kidney Ca, Clear cell type (ODO4340) | 0.0 | 0.0 |
| 83791 Kidney NAT (ODO4340) | 100.0 34.4 | |
| 83792 Kidney Ca, Nuclear grade 3 (ODO4348) | 2.0 | 4.9 |
| 83793 Kidney NAT (ODO4348) | 30.2 | 19.9 |
| 87474 Kidney Cancer (ODO4622-01) | 0.0 | 2.4 |
| 87475 Kidne NAT (ODO4622-03) | 8.4 | 7.2 |
| 85973 Kidney Cancer (ODO4450-01) | 0.0 | 0.0 |
| 85974 Kidney NAT (ODO4450-03) | 47.3 | 12.9 |
| Kidney Cancer Clontech 8120607 | 0.0 | 0.0 |
| Kidney NAT Clontech 8120608 | 0.0 | 0.0 |
| Kidney Cancer Clontech 8120613 | 0.0 | 0.0 |
| Kidney NAT Clontech 8120614 | 20.6 | 22.9 |
| Kidney Cancer Clontech 9010320 | 0.0 | 0.0 |
| Kidney NAT Clontech 9010321 | 3.4 | 26.4 |
| Normal Uterus GENPAK 061018 | 0.0 | 0.0 |
| Uterus Cancer GENPAK 064011 | 14.9 | 0.0 |
| Normal Thyroid Clontech A+ 6570-1 | 0.0 | 0.0 |
| Thyroid Cancer GENPAK 064010 | 0.0 | 0.0 |
| Thyroid Cancer INVITROGEN A3021 52 | 0.0 | 0.0 |
| Thyroid NAT INVITROGEN A302153 | 0.0 | 0.0 |
| Normal Breast GENPAK 061019 | 5.2 | 3.5 |
| 84877 Breast Cancer ODO4566) | 0.0 | 0.0 |
| 85975 Breast Cancer (ODO4590-01) | 0.0 | 0.0 |
| 85976 Breast Cancer Mets (ODO4590-03) | 0.0 | 0.0 |
| 87070 Breast Cancer Metastasis (ODO4655-05) | 0.0 | 0.0 |
| GENPAK Breast Cancer 064006 | 0.0 | 2.5 |
| Breast Cancer Clontech 9100266 | 6.2 | 0.0 |
| Breast NAT Clontech 9100265 | 0.0 | 0.0 |
| Breast Cancer INVITROGEN A209073 | 1.5 | 2.5 |
| Breast NAT INVITROGEN A2090734 | 24.3 | 26.2 |
| Normal Liver GENPAK 061009 | 10.5 | 2.7 |
| Liver Cancer GENPAK 064003 | 5.9 | 1.7 |
| Liver Cancer Research Genetics RNA 1025 | 21.6 | 11.0 |
| Liver Cancer Research Genetics RNA 1026 | 0.0 | 0.0 |
| Paired Liver Cancer Tissue Research Genetics | 3.3 | 13.5 |
| RNA 6004-T | | |
| Paired Liver Tissue Research Genetics RNA 6004-N | 3.2 | 1.4 |
| Paired Liver Cancer Tissue Research Genetics | 0.0 | 0.0 |
| RNA 6005-T | | |
| Paired Liver Tissue Research Genetics RNA 6005-N | 0.0 | 0.0 |
| Normal Bladder GENPAK 061001 | 0.0 | 0.0 |
| Bladder Cancer Research Genetics RNA 1023 | 0.0 | 0.0 |
| Bladder Cancer INVITROGEN A302173 | 4.6 | 2.3 |
| 87071 Bladder Cancer (ODO471 8-01) | 17.9 | 11.4 |
| 87072 Bladder Normal Adjacent (OD0471 8-03) 0.0 | 0.0 | |
| Normal Ovary Res. Gen. | 0.0 | 0.0 |
| Ovarian Cancer GENPAK 064008 | 1.7 | 4.8 |
| 87492 Ovary Cancer (ODO4768-07) | 0.0 | 2.1 |
| 87493 Ovary NAT (ODO4768-08) | 0.0 | 0.0 |
| Normal Stomach GENPAK 061017 | 3.3 | 2.9 |
| NAT Stomach Clontech 9060359 | 0.0 | 0.0 |
| Gastric Cancer Clontech 9060395 | 0.0 | 0.0 |
| NAT Stomach Clontech 9060394 | 0.0 | 0.0 |
| Gastric Cancer Clontech 9060397 | 0.0 | 0.0 |
| NAT Stomach Clontech 9060396 | 0.0 | 0.0 |
| Gastric Cancer GENPAK 064005 | 6.3 | 3.8 |

TABLE 18
Taqman Results for clone 27455183.0.19 (Panel 4D)

| | Panel 4D | |
|---|---|---|
| Tissue_Name | Run 1 | Run 2 |
| 93768_Secondary Th1_anti-CD28/anti-CD3 | 0.0 | 0.0 |
| 93769_Secondary Th2_anti-CD28/anti-CD3 | 0.0 | 0.0 |
| 93770_Secondary Tr1_anti-CD28lanti-CD3 | 13.5 | 17.1 |
| 93573_Secondary Th1_resting day 4-6 in IL-2 | 0.0 | 0.0 |
| 93572_Secondary Th2_resting day 4-6 in IL-2 | 0.0 | 0.0 |
| 93571_Secondary Tr_resting day 4-6 in IL-2 | 0.0 | 0.0 |
| 93568_primary Th1_anti-CD28/anti-CD3 | 0.0 | 0.0 |
| 93569_primary Th2_anti-CD28/anti-CD3 | 0.0 | 0.0 |
| 93570_primary Tr1_anti-CD28/anti-CD3 | 0.0 | 0.0 |
| 93565_primary Th1_resting dy 4-6 in IL-2 | 0.0 | 0.0 |
| 93566_primary Th2_resting dy 4-6 in lL-2 | 0.0 | 0.0 |
| 93567_primary Tr1_resting dy 4-6 in IL-2 | 0.0 | 0.0 |
| 93351_CD45RA CD4 lymphocyte_anti-CD28/anti-CD3 | 0.0 | 0.0 |
| 93352_CD45RO CD4 lymphocyte_anti-CD28/anti-CD3 | 0.0 | 0.0 |
| 93251_CD8 Lymphocytes_anti-CD28/anti-CD3 | 0.0 | 0.0 |
| 93353_chronic CD8 Lymphocytes 2ry_resting dy 4-6 in IL-2 | 0.0 | 0.0 |
| 93574_chronic CD8 Lymphocytes 2ry_activated CD3/CD28 | 0.0 | 0.0 |
| 93354_CD4_none | 5.8 | 0.0 |
| 93252_Secondary Th1/Th2/Tr1_anti-CD95 CH11 | 10.0 | 0.0 |
| 93103_LAK cells_resting | 0.0 | 0.0 |
| 93788_LAK cells_IL-2 | 0.0 | 0.0 |
| 93787_LAK cells_IL-2 + IL-12 | 0.0 | 0.0 |
| 93789_LAK cells_IL-2 + IFN gamma | 0.0 | 0.0 |
| 93790_LAK cells IL-2 + IL-18 | 0.0 | 0.0 |
| 93104_LAK cells_PMA/ionomycin and IL-18 | 0.0 | 0.0 |
| 93578_NK Cells IL-2_resting | 0.0 | 0.0 |
| 93109_Mixed Lymphocyte Reaction_Two Way MLR | 0.0 | 0.0 |
| 93110_Mixed Lymphocyte Reaction_Two Way MLR | 0.0 | 0.0 |
| 93111_Mixed Lymphocyte Reaction_Two Way MLR | 0.0 | 0.0 |
| 93112_Mononuclear Cells (PBMCs)_resting | 0.0 | 0.0 |
| 93113_Mononuclear Cells (PBMCs)_PWM | 0.0 | 0.0 |
| 93114_Mononuclear Cells (PBMCs)_PHA-L | 0.0 | 0.0 |
| 93249_Ramos (B cell)_none | 0.0 | 38.2 |
| 93250_Ramos (B cell)_ionomycin | 0.0 | 0.0 |
| 93349_B lymphocytes_PWM | 0.0 | 68.8 |
| 93350_B lymphoytes_CD40L and IL-4 | 31.0 | 0.0 |
| 92665_EOL-1 (Eosinophil)_dbcAMP differentiated | 0.0 | 0.0 |
| 93248_EOL-1 (Eosinophil)_dbcAMP/PMAionomycin | 0.0 | 0.0 |
| 93356_Dendritic Cells_none | 0.0 | 0.0 |
| 93355_Dendritic Cells_LPS 100 ng/ml | 0.0 | 0.0 |
| 93775_Dendritic Cells_anti-CD40 | 32.5 | 0.0 |
| 93774_Monocytes_resting | 0.0 | 0.0 |
| 93776_Monocytes_LPS 50 ng/ml | 0.0 | 0.0 |
| 93581_Macrophages_resting | 0.0 | 0.0 |
| 93582_Macrophages_LPS 100 ng/ml | 0.0 | 0.0 |
| 93098_HUVEC (Endothelial)_none | 0.0 | 0.0 |
| 93099_HUVEC (Endothelial)_starved | 11.3 | 0.0 |
| 93100_HUVEC (Endothelial)_IL-1b | 0.0 | 14.6 |
| 93779_HUVEC (Endothelial)_IFN gamma | 0.0 | 0.0 |
| 93102_HUVEC (Endothelial)_TNF alpha + IFN gamma | 0.0 | 0.0 |
| 93101_HUVEC (Endothelial)_TNF alpha + IL4 | 0.0 | 0.0 |
| 93781_HUVEC (Endothelial)_IL-11 | 0.0 | 0.0 |
| 93583_Lung Microvascular Endothelial Cells_none | 0.0 | 0.0 |
| 93584_Lung Microvascular Endothelial Cells_TNFa | 0.0 | 0.0 |
| (4 ng/ml) and IL1b (1 ng/ml) | | |
| 92662_Microvascular Dermal endothelium_none | 0.0 | 0.0 |
| 92663_Microsvasular Dermal endothelium_TNFa | 0.0 | 0.0 |
| (4 ng/ml) and ILlb (1 ng/ml) | | |
| 93773_Bronchial epithelium_TNFa | 0.0 | 0.0 |
| (4 ng/ml) and IL1b (1 ng/ml) ** | | |
| 93347_Small Airway Epithelium_none | 0.0 | 0.0 |
| 93348_Small Airway Epithelium_TNFa (4 ng/ml) and | 0.0 | 0.0 |
| IL1b (1 ng/ml) | | |
| 92668_Coronery Artery SMC_resting | 0.0 | 0.0 |
| 92669_Coronery Artery SMC_TNFa | 0.0 | 0.0 |
| (4 ng/ml) and IL1b (1 ng/ml) | | |
| 93107_astrocytes_resting | 0.0 | 0.0 |
| 93108_astroces_TNFa 4 ng/ml and IL1b 1 ng/ml | 0.0 | 0.0 |
| 92666_KU-812 (Basophil)_resting | 0.0 | 40.3 |
| 92667_KU-812 (Basophil)_PMA/ionoycin | 0.0 | 0.0 |
| 93579_CCD1106 (Keratinocytes)_none | 0.0 | 0.0 |
| 93580_CCD1106 (Keratinocytes)_TNFa and IFNg ** | 0.0 | 0.0 |
| 93791_Liver Cirrhosis | 100.0 | 99.3 |
| 93792_Lupus Kidney | 0.0 | 0.0 |
| 93577_NCI-H292 | 0.0 | 0.0 |
| 93358_NCI-H292_IL-4 | 0.0 | 0.0 |
| 93360_NCI-H292_IL-9 | 10.6 | 0.0 |
| 93359_NCI-H292_IL-13 | 0.0 | 65.5 |
| 93357_NCI-H292_IFN gamma | 0.0 | 24.8 |
| 93777_HPAEC_- | 0.0 | 0.0 |
| 93778_HPAEC_IL-1 beta/TNA alpha | 0.0 | 0.0 |
| 93254_Normal Human Lung Fibroblast_none | 0.0 | 0.0 |
| 93253_Normal Human Lung Fibroblast_TNFa (4 ng/ml) | 0.0 | 0.0 |
| and IL-1b (1 ng/ml) | | |
| 93257_Normal Human Lung Fibroblast_IL-4 | 0.0 | 0.0 |
| 93256_Normal Human Lung Fibroblast_IL-9 | 0.0 | 0.0 |
| 93255_Normal Human Lung Fibroblast_IL-13 | 0.0 | 0.0 |
| 93258_Normal Human Lung Fibroblast_IFN gamma | 0.0 | 0.0 |
| 93106_Dermal Fibroblasts CCD1070_resting | 0.0 | 0.0 |
| 93361_Dermal Fibroblasts CCD1070_TNF alpha 4 ng/ml | 0.0 | 43.8 |
| 93105_Dermal Fibroblasts CCD1070_IL-1 beta 1 ng/ml | 0.0 | 0.0 |
| 93772_dermal fibroblast_IFN gamma | 42.0 | 27.7 |
| 93771_dermal fibroblast_IL-4 | 10.7 | 90.1 |
| 93259_IBD Colitis 1** | 0.0 | 0.0 |
| 93260_IBD Colitis 2 | 13.8 | 0.0 |
| 93261_IBD Crohns | 0.0 | 46.7 |
| 735010_Colon_normal | 15.6 | 0.0 |
| 735019_Lung_none | 112.9 | 116.8 I |
| 64028-1_Thymus_none | 69.3 | 100.0 |
| 64030-1_Kidney_none | 0.0 | 0.0 |

Taqman results in Table 18 demonstrate that clone FCTR6 is differentially expressed in clear cell Renal cell carcinoma tissues versus the normal adjacent kidney tissues and thus could have a potential role in the treatment of renal cell carcinoma.

### EQUIVALENTS

Although particular embodiments have been disclosed herein in detail, this has been done by way of example for purposes of illustration only, and is not intended to be limiting with respect to the scope of the appended claims which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims.

What is claimed is:

1. An isolated polypeptide comprising an amino acid sequence selected from the group consisting of:
(a) a mature form of an amino acid sequence selected from the group consisting of SEQ ID NOS:2, 4, 6, 8, 13, 15, 17, 19, 21, 23, and 25;
(b) a variant of a mature form of an amino acid sequence selected from the group consisting of SEQ ID NOS:2, 4, 6, 8, 13, 15, 17, 19, 21, 23, and 25, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NOS:2, 4, 6, 8, 13, 15, 17, 19, 21, 23, and 25; and
(d) a variant of an amino acid sequence selected from the group consisting of SEQ ID NOS:2, 4, 6, 8, 13, 15, 17, 19, 21, 23, and 25, wherein one or more amino acid residues in said variant differs from the amino acid sequence of said mature form, provided that said variant differs in no more than 15% of amino acid residues from said amino acid sequence.

2. The polypeptide of claim 1, wherein said polypeptide comprises the amino acid sequence of a naturally-occurring allelic variant of an amino acid sequence selected from the group consisting of SEQ ID NOS:2, 4, 6, 8, 13, 15, 17, 19, 21, 23, and 25.

3. The polypeptide of claim 2, wherein said allelic variant comprises an amino acid sequence that is the translation of a nucleic acid sequence differing by a single nucleotide from a nucleic acid sequence selected from the group consisting of SEQ ID NOS: 1, 3, 5, 7, 9, 10, 11, 12, 14, 16, 18, 20, 22, and 24.

4. The polypeptide of claim 1, wherein the amino acid sequence of said variant comprises a conservative amino acid substitution.

5. An isolated nucleic acid molecule comprising a nucleic acid sequence encoding a polypeptide comprising an amino acid sequence selected from the group consisting of:
(a) a mature form of an amino acid sequence selected from the group consisting of SEQ ID NOS:2, 4, 6, 8, 13, 15, 17, 19, 21, 23, and 25;
(b) a variant of a mature form of an amino acid sequence selected from the group consisting of SEQ ID NOS:2, 4, 6, 8, 13, 15, 17, 19, 21, 23, and 25, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NOS:2, 4, 6, 8, 13, 15, 17, 19, 21, 23, and 25;
(d) a variant of an amino acid sequence selected from the group consisting of SEQ ID NOS:2, 4, 6, 8, 13, 15, 17, 19, 21, 23, and 25, wherein one or more amino acid residues in said variant differs from the amino acid sequence of said mature form, provided that said variant differs in no more than 15% of amino acid residues from said amino acid sequence;
(e) a nucleic acid fragment encoding at least a portion of a polypeptide comprising an amino acid sequence chosen from the group consisting of SEQ ID NOS:2, 4, 6, 8, 13, 15, 17, 19, 21, 23, and 25, or a variant of said polypeptide, wherein one or more amino acid residues in said variant differs from the amino acid sequence of said mature form, provided that said variant differs in no more than 15% of amino acid residues from said amino acid sequence; and
(f) a nucleic acid molecule comprising the complement of (a), (b), (c), (d) or (e).

6. The nucleic acid molecule of claim 5, wherein the nucleic acid molecule comprises the nucleotide sequence of a naturally-occurring allelic nucleic acid variant.

7. The nucleic acid molecule of claim 5, wherein the nucleic acid molecule encodes a polypeptide comprising the amino acid sequence of a naturally-occurring polypeptide variant.

8. The nucleic acid molecule of claim 5, wherein the nucleic acid molecule differs by a single nucleotide from a nucleic acid sequence selected from the group consisting of SEQ ID NOS:1, 3, 5, 7, 9, 10, 11, 12, 14, 16, 18, 20, 22, and 24.

9. The nucleic acid molecule of claim 5, wherein said nucleic acid molecule comprises a nucleotide sequence selected from the group consisting of:
(a) a nucleotide sequence selected from the group consisting of SEQ ID NOS: 1, 3, 5, 7, 9, 10, 11, 12, 14, 16, 18, 20, 22, and 24;
(b) a nucleotide sequence differing by one or more nucleotides from a nucleotide sequence selected from the group consisting of SEQ ID NOS:1, 3, 5, 7, 9, 10, 11, 12, 14, 16, 18, 20, 22, and 24, provided that no more than 20% of the nucleotides differ from said nucleotide sequence;
(c) a nucleic acid fragment of (a); and
(d) a nucleic acid fragment of (b).

10. The nucleic acid molecule of claim 5, wherein said nucleic acid molecule hybridizes under stringent conditions to a nucleotide sequence chosen from the group consisting of SEQ ID NOS: 1, 3, 5, 7, 9, 10, 11, 12, 14, 16, 18, 20, 22, and 24, or a complement of said nucleotide sequence.

11. The nucleic acid molecule of claim 5, wherein the nucleic acid molecule comprises a nucleotide sequence selected from the group consisting of:
(a) a first nucleotide sequence comprising a coding sequence differing by one or more nucleotide sequences from a coding sequence encoding said amino acid sequence, provided that no more than 20% of the nucleotides in the coding sequence in said first nucleotide sequence differ from said coding sequence;
(b) an isolated second polynucleotide that is a complement of the first polynucleotide; and
(c) a nucleic acid fragment of (a) or (b).

12. A vector comprising the nucleic acid molecule of claim 11.

13. The vector of claim 12, further comprising a promoter operably-linked to said nucleic acid molecule.

14. A cell comprising the vector of claim 12.

15. An antibody that binds immunospecifically to the polypeptide of claim 1.

16. The antibody of claim 15, wherein said antibody is a monoclonal antibody.

17. The antibody of claim 15, wherein the antibody is a humanized antibody.

18. A method for determining the presence or amount of the polypeptide of claim 1 in a sample, the method comprising:
(a) providing the sample;
(b) contacting the sample with an antibody that binds immunospecifically to the polypeptide; and
(c) determining the presence or amount of antibody bound to said polypeptide, thereby determining the presence or amount of polypeptide in said sample.

19. A method for determining the presence or amount of the nucleic acid molecule of claim 5 in a sample, the method comprising:
(a) providing the sample;
(b) contacting the sample with a probe that binds to said nucleic acid molecule; and
(c) determining the presence or amount of the probe bound to said nucleic acid molecule, thereby determining the presence or amount of the nucleic acid molecule in said sample.

20. The method of claim 19 wherein presence or amount of the nucleic acid molecule is used as a marker for cell or tissue type.

21. The method of claim 20 wherein the cell or tissue type is cancerous.

22. A method of identifying an agent that binds to a polypeptide of claim 1, the method comprising:
(a) contacting said polypeptide with said agent; and
(b) determining whether said agent binds to said polypeptide.

23. The method of claim 22 wherein the agent is a cellular receptor or a downstream effector.

24. A method for identifying an agent that modulates the expression or activity of the polypeptide of claim 1, the method comprising:
(a) providing a cell expressing said polypeptide;
(b) contacting the cell with said agent, and
(c) determining whether the agent modulates expression or activity of said polypeptide,
whereby an alteration in expression or activity of said peptide indicates said agent modulates expression or activity of said polypeptide.

25. A method for modulating the activity of the polypeptide of claim 1, the method comprising contacting a cell sample expressing the polypeptide of said claim with a compound that binds to said polypeptide in an amount sufficient to modulate the activity of the polypeptide.

26. A method of treating or preventing a FCTRX-associated disorder, said method comprising administering to a subject in which such treatment or prevention is desired the polypeptide of claim 1 in an amount sufficient to treat or prevent said FCTRX-associated disorder in said subject.

27. The method of claim 26 wherein the disorder is a neurodegenerative disorder.

28. The method of claim 26 wherein the disorder is related to cell signal processing and metabolic pathway modulation.

29. The method of claim 26, wherein said subject is a human.

30. A method of treating or preventing a FCTRX-associated disorder, said method comprising administering to a subject in which such treatment or prevention is desired the nucleic acid of claim 5 in an amount sufficient to treat or prevent said FCTRX-associated disorder in said subject.

31. The method of claim 30 wherein the disorder is a neurodegenerative disorder.

32. The method of claim 30 wherein the disorder is related to cell signal processing and metabolic pathway modulation.

33. The method of claim 30, wherein said subject is a human.

34. A method of treating or preventing a FCTRX-associated disorder, said method comprising administering to a subject in which such treatment or prevention is desired the antibody of claim 15 in an amount sufficient to treat or prevent said FCTRX-associated disorder in said subject.

35. The method of claim 34 wherein the disorder is selected from the group consisting of Also within the scope of the invention is the use of a Therapeutic in the manufacture of a medicament for treating or preventing disorders or syndromes including, e.g., Colorectal cancer, adenomatous polyposis coli, myelogenous leukemia, congenital ceonatal alloimmune thrombocytopenia, multiple human solid malignancies, malignant ovarian tumours particularly at the interface between epithelia and stroma, malignant brain tumors, mammary tumors, human gliomas, astrocytomas, mixed glioma/astrocytomas, renal cells carcinoma, breast adenocarcinoma, ovarian cancer, melanomas, renal cell carcinoma, clear cell and granular cell carcinomas, autocrine/paracrine stimulation of tumor cell proliferation, autocrine/paracrine stimulation of tumor cell survival and tumor cell resistance to cytotoxic therapy, paranechmal and basement membrane invasion and motility of tumor cells thereby contributing to metastasis, tumor-mediated immunosuppression of T-cell mediated immune effector cells and pathways resulting in tumor escape from immune surveilance, neurological disorders, neurodegenerative disorders, nerve trauma, familial myelodysplastic syndrome, Charcot-Marie-Tooth neuropathy, demyelinating Gardner syndrome, familial myelodysplastic syndrome; mental health conditions, immunological disorders, allergy and infection, asthma, bronchial asthma, Avellino type eosinophilia, lung diseases, reproductive disorders, male infertility, female reproductive system disorders, male and female reproductive diseases, hemangioma, deafness, glycoprotein Ia deficiency, desmoid disease, turcot syndrome, liver cirrhosis, hepatitis C, gastric disorders, pancreatic diseases like diabetes, Schistosoma mansoni infection, Spinocerebellar ataxia, Plasmodium falciparum parasitemia, Corneal dystrophy—Groenouw type I, Corneal dystrophy—lattice type I, and Reis-Bucklers corneal dystrophy.

36. The method of claim 34 wherein the disorder is related to cell signal processing and metabolic pathway modulation.

37. The method of claim 34, wherein the subject is a human.

38. A pharmaceutical composition comprising the polypeptide of claim 1 and a pharmaceutically-acceptable carrier.

39. A pharmaceutical composition comprising the nucleic acid molecule of claim 5 and a pharmaceutically-acceptable carrier.

40. A pharmaceutical composition comprising the antibody of claim 15 and a pharmaceutically-acceptable carrier.

41. A kit comprising in one or more containers, the pharmaceutical composition of claim 38.

42. A kit comprising in one or more containers, the pharmaceutical composition of claim 39.

43. A kit comprising in one or more containers, the pharmaceutical composition of claim 40.

44. A method for determining the presence of or predisposition to a disease associated with altered levels of the polypeptide of claim 1 in a first mammalian subject, the method comprising:
(a) measuring the level of expression of the polypeptide in a sample from the first mammalian subject; and
(b) comparing the amount of said polypeptide in the sample of step (a) to the amount of the polypeptide present in a control sample from a second mammalian subject known not to have, or not to be predisposed to, said disease;
wherein an alteration in the expression level of the polypeptide in the first subject as compared to the control sample indicates the presence of or predisposition to said disease.

45. The method of claim 44 wherein the predisposition is to cancers.

46. A method for determining the presence of or predisposition to a disease associated with altered levels of the nucleic acid molecule of claim 5 in a first mammalian subject, the method comprising:
(a) measuring the amount of the nucleic acid in a sample from the first mammalian subject; and
(b) comparing the amount of said nucleic acid in the sample of step (a) to the amount of the nucleic acid present in a control sample from a second mammalian subject known not to have or not be predisposed to, the disease;
wherein an alteration in the level of the nucleic acid in the first subject as compared to the control sample indicates the presence of or predisposition to the disease.

47. The method of claim 46 wherein the predisposition is to cancers.

48. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising an amino acid sequence of at least one of SEQ ID NOS:2, 4, 6, 8, 13, 15, 17, 19, 21, 23, and 25, or a biologically active fragment thereof.

49. A method of treating a pathological state in a mammal, the method comprising administering to the mammal the antibody of claim 15 in an amount sufficient to alleviate the pathological state.
